(19) United States
(12) Patent Application Publication
(10) Pub. No.: US 2017/0003277 A1
(43) Pub. Date: Jan. 5, 2017

(54) BIOLOGICAL CHARACTERIZATION OF A GLATIRAMER ACETATE RELATED DRUG PRODUCT USING MAMMALIAN AND HUMAN CELLS

(71) Applicants: Michael Hayden, Petach-Tikva (IL); Fadi George Towfic, Somerville, MA (US); Sarah Elisabeth Kolitz, Cambridge, MA (US); Benjamin James Zeskind, Cambridge, MA (US); David Ladkani, Kiriat Yovel (IL); Tal Hasson, Raanana (IL); Liat Hayardeny, Tel Aviv (IL); Iris Grossman, Yakir (IL)

(72) Inventors: Michael Hayden, Petach-Tikva (IL); Fadi George Towfic, Somerville, MA (US); Sarah Elisabeth Kolitz, Cambridge, MA (US); Benjamin James Zeskind, Cambridge, MA (US); David Ladkani, Kiriat Yovel (IL); Tal Hasson, Raanana (IL); Liat Hayardeny, Tel Aviv (IL); Iris Grossman, Yakir (IL)

(21) Appl. No.: 14/789,833

(22) Filed: Jul. 1, 2015

(51) Int. Cl. G01N 33/50; A61K 31/785; G01N 33/94

(52) U.S. Cl. CPC G01N 33/5023; G01N 33/9493; A61K 31/785

(57) ABSTRACT

The present invention provides a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) i) determining the level of expression of at least one gene selected from the group consisting of ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, IL10, LPHN1, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, and ZFAND6; ii) determining the level of expression of at least one gene selected from the group consisting of BIRC3, CCL24, CCR1, CISH, CSF1R, CX3CR1, CXCL10, HSPD1, ICAM1, IL1B, IFNGR1, IL27, IL2RG, IL7R, IL1RN, MMP1, MMP9, MMP14, PGRMC1, PRDM1, CARD15, CCL2, CCL5, CD14, IL10, THBD, and NFKBIA, wherein if IL1B, IL10, or MMP9 is the at least one gene selected in part (c)(ii), then selecting at least a second different gene from the group other than IL1B, IL10, or MMP9; iii) determining the level of expression of at least one gene selected from the group consisting of C13ORF31, C14ORF10, C1ORF51, C1ORF63, CBR4, CB36, CD9, COL6A1, DAB2, GATA2, KIAA0907, LOC100506233, MCM6, MMP1, MS4A4A, MTSS1, PCMTD1, STK4, STX7, TAF15, TARP, TIA1, TMF1, TRGC2, TXNDC11, and ZCCHC7, and ZCCHC7; iv) determining the level of expression of at least one gene selected from the group consisting of ANXA1, ARRB2, BEAN, BIN1, C1ORF63, CD44, CD9, CFP, COL6A1, CRIP2, EPB41, Fam119a, FGR, FOX03B, HSD11B1, HSPD1P6, LOC387790, MPEG1, MYB, OLIG1, PLD1, PPP4R2, PRDM1, RBM6, SNX27, 5OD2, STATH, TARP, TREM1, TRGC2, UBN2, and ZCCHC7; v) determining the level of expression of at least one gene selected from the group consisting of ADAM9, ADAMDEC1, AKR1C2, ANXA2, ANXA2P2, ARHGAP18, ARHGAP18, ARL6IP5, ARL6IP5, ATP2C1, BID, BIRC3, BTG1, CARD15, C1ORF21, C13ORF31, C5ORF13, C5ORF32, C9ORF130, CAST, CCL2, CCL5, CD14, CD300A, CD36, CD40, CD55, CD9, CENTA2, CHST11, COL6A1, CRYBB2, CXCL10, CYLD, DAB2, EBI3, EBI2, ECOP, EGF, FABP4, FXYD2, GHRL, GIMAP8, GLIPR1, G0S2, HMGB2, HNRPLL, ICAM1, ICAM2, IFIH1, IFNGR1, IL10, MORA, IL4I1, INADL, ISG20, ITGB5, KIAA1505, KYNU, LACTB, L0054103, LOC388344, LOC652751, LPAAT-THETA, LPXN, MAFB, MALT1, MFI2, MGC5618, MGLL, MITF, MLF1, MMPI, MMP9, MPEG1, MTSS1, MXD1, NT5E, NFKBIE, NFKBIA, NFE2L3, NFE2L3, OSBPL11, P2RX4, P2RY5, PLEKHO1, POPDC3, PLAUR, PRDM1, PSCDBP, PTX3, RAB27B, RCSD1, RPL13, SGIP1, SLC39A8, SNORD68, SRPX2, SRA1, SLIC1, SLAMF8, SLIC1, SOD2, STATH, STEAP1, SYNJ2, SYNJ2, TATDN3, TGM5, THBD, TNFAIP3, TNFAIP6, TNFRSF9, TNFSF13B, TPSAB1, TPSB2, TREM1, TXNL2, VPS33A, and VSNL1; vi) determining the level of expression of at least one gene selected from the group consisting of ACTN4, BTBD14A, C14ORF10, CISH, CLK1, CRLF3, FAM62A, FBX045, GAPDHS, HDAC4, HIC2, HNRPD, HSPD1, LOC648342, MYB, NAPB, OXCT2, SERPINB2, SFRS14, SPFH1, STT3B, WDFY1, ZNF250, and ZNF566; vii) determining the level of expression of at least one gene selected from the group consisting of A2M, ABCB1, ABCC3, ABHD2, ACPP, ADAMDEC1, ADFP, ADORA2B, ADORA3, AHNAK, ALCAM, ANKH, ANKRD57, ANXA2, ANXA2P2, APBB1IP, AQP1, ARHGAP18, ARHGAP20, ARHGEF3, ARID5B, ARMC9, ATF5, ATP1B1, ATP6V0D2, ATP9A, ATP10A, AYTL1, BCL2A1, BCL6, C3AR1, C13ORD31, C9ORF88, C9ORF89, C1ORF21, C1ORF21, C10ORF95, C13ORF31, C21ORF7, CARD12, CARD15, CCDC83, CCL5, CCL24, CCND1, CCR1, CD9, CD36, CD52, CD86, CD109, CD180, CD244, CDK5RAP2, CDKN1A, CENTA2, CKB, CKLF, CLEC7A, CNIH3, COL6A1, COL22A1, CRIP2, CSF1R, CSPG4, CTSL, CTSLL3, CX3CR1, CXCR7, CYBB, CYP1B1, DAB2, DAPP1, DDIT4L, DIXDC1, DOCK4, DOK2, DKFZP56400823, DKFZP686O1327, EBI2, EMP1, EMR2, ENPP2, EPAS1, EPS8, EPSTI1, EVL, FABP4, FADS3, FAM26B, FGD2, FGD2, FGD4, FGL2, FN1, FTH1, GBP2, GBP3, GBP5, GCNT1, GDPD1, GNDL, GNLY, GLIPR1, GLIS3, GPC1, GPR35, H2A/R, HAVCR2, HMCN1, HIPK2, HIST2H2AA, HIVEP1, HMOX1, HSPB7, ICAM1, ID2, ID2B, IFI30, IFI44, IFNGR1, IGFBP3, IL2RG, IL4I1, IL1ORA, IL27RA, IL7R, IL10, INA, IRF7, ITGB5, ITGB7, KIAA1505, KIAA1706, KMO, LBH, LFNG, LILRB1, LILRB2, LMNA, L0051334, LOC201895, LOC284262, L0051334, LOC643424, LOC643834, LOC643847, LOC644242, LOC645238, LOC650429, LOC650446, LOC652543, LOC653610, LOC653754, LPAAT-THETA, LPXN, MAF, MAFB, MAML2, MAML3, MARCH1, MCOLN3, MDGA1, ME1, MFI1, MFI2, MGC45491, MGLL, MITF, MMP1, MMP2, MMP9, MMP14, MMP19, MTMR11, MTSS1, MTSUl, NGEF, NME7, NPTX1, NRCAM, NRP1, NRP2, NT5E, OAS1, OLR1, P2RY5, P2RY14, PALLD, PAPSS2, PAQR5, PCDHGA1, PCDHGA2, PCDHGA3, PCDHGA4, PCDHGA5, PCDHGA6, PCDHGA7, PCDHGA8, PCDHGA9, PCDHGA10, PCDHGA11, PCDHGA12, PCDHGB1, PCDHGB2, PCDHGB3, PCDHGB4, PCDHGB5, PCDHGB7, PCDHGC3, PCDHGC4, PCDHGC5, PDK4, PDLIM7, PFKFB4, PGA5, PDLIM4, PHLDA1, PLA2G4A, PLEKHA7, PLEKHO1, POPDC3, PRDM1, PRSS23, PSCDBP, PSD3, PTAFR, PTGS1, PTPRO, PTRF, PTX3, RAB27B, RAB38, RAB7B, RAPH1, RASGRF1, RGL1, RGS13, RHBDF1, RIN2, S100A2, SART2, SERPINE2, SERTAD1, SETBP1, SGIP1, SH3TC1, SKIL, SLA, SLAMF7, SLAMF8, SLC6AS, SLC7A11, SLC12A6, SLC37A2, SLC41A2, SLC38A6, SLC43A2, SNAI3, ST3GAL5, STATH, STEAP1, SUCNR1, SYTL1, TBXAS1, TCF4, TFAP2A, THBD, TLR4, TM7SF4, TMEM39A, TMEM158, TNCRNA, TNFSF13B, TNFRSF21, TREM1, TRIM22, TRPA1, TRPM8, TRPS1, TUBB2A, UBXD5, UGCG, UPP1, VASH1, VEGF, VSNL1, and ZFP36L1; viii) determining the level of expression of at least one gene selected from the group consisting of ABCG1, ADAMTS1, ANKRD41, ANXA3, APCDD1, BCL2, BCL11A, BMP8B, C1ORF71, C1ORF76, ClORD121, C12ORF24, C16ORF73, C16ORF74, C20ORD27, C20ORF103, C20ORF112, CACNA2D3, CAMTA1, CAV1, CCDC85B, CDCA7L, CEBPD, CKAP4, CNTN4, COL8A2, CSPG5, CXCR4, DCUN1D4, DEPDC6, DMRT2, DUSP2, DZIP1, EBF3, EGR4, FAM117A, FKBP4, FL135848, FLOT2, GFI1, GMDS, GPR18, HAL, HNF4G, HSPC049, IL17D, IRX3, KBTBD11, KCNQ4, KCTD15, KIAA0146, KIAA0984, KIAA1026, KIAA1553, KLHL23, LGR4, LOC201164, LOC284454, LOC387763, LOC642083, LOC648232, MGC2408, MICAL1, MID1IP1, MSRB3, MUC19, NAPSB, NR1D2, PCDH8, PDE4B, PDGFD, PER2, PHF15, PKP2, PLK2, OAF, OSBPL1A, OSR2, OXCT2, PGM1, PMAIP1, PNMA6A, POU4F2, PSAT1, RAB33A, RASGRP2, RBM38, RET, RFTN1, SERPINB2, SERPINB10, SLAIN1, SLC1A3, SLC16A1, SLC19A1, SLC27A2, SLC29A1, SLC39A14, SLCO4A1, SNF1LK, SOX12, SPFH1, SPRY1, STEAP3, 5YDE2, SYNPO2, TARP, TEAD4, TDRD7, TMEM67, TPD52, TRGC2, TRGV2, TRGV9, TRIB3, TSPAN2, TUBA1, VIT, WDR49, WNT3, WT1, and YES1; ix) determining the level of expression of at least one gene selected from the group consisting of AHRR, CCDC36, CYP1B1, DOC1, EPB41L3, GAS7, GPR68, NPTX1, PDCD6, and TIPARP; x) determining the level of expression of at least one gene selected from the group consisting of ADRB2, COTL1, LOC285758, LOC644137, MALAT1, PRG1, RNF43, SAT1, THAP5, TIMP3, and TSC22D1; xi) determining the level of expression of at least one gene selected from the group consisting of AW011738, Bst2, Daxx, Gm16340, Hck, Herc6, Ifi202b, Ifi203, Ifi204, Ifi44, Ifi441, Ifit2, Inpp5b, LOC100044068, LOC100862473, Mx1, Oas11, Phf11d, Oyhin1, Sdc3, Setdb2, Tor3a, Usp18, and Zcchc2; xii) determining the level of expression of at least one gene selected from the group consisting of Ccdc711, D13ERTD608E, Fads2, Gm2a, Ifi2711, Ighm, Klk1, Scd2, Siglech, and Tspan2; xiii) determining the level of expression of at least one gene selected from the group consisting of Ahrr, AI607873, Atp10a, AW011738, Casp44, Cxc13, Gm9706, Ifi202b, Ifit2, Ifitm6, I118, Lcn2, LOC100044068, Ms4a6d, Mx1, Papd7, Rsad2, Slfn3, Slfn4, Tdrd7, Tiparp, and Zcchc2; xiv) determining the level of expression of at least one gene selected from the group consisting of Aldoc, Casp6, Ccdc711, Cox7a1, Egln3, Fam162a, Gfi1, Gpi1, Grhpr, Ifi2711, Ighm, Kcnq5, Klhdc2, Pgk1, Pkm, Tpi1, and Trappc6a; xv) determining the level of expression of at least one gene selected from the group consisting of 1600014C10Rik, 2810474O19Rik, 6720475J19Rik, Adam8, Adar, Agrn, Ahrr, AI607873, Amigo2, Ankfy1, Apobec1, Arf4, Asb2, Ascc3, Atp10a, Atp8b4, AW011738, B4galt5, BC147527, Bst2, Casp4, Chic1, Cmpk2, Csprs, Cxc13, Cybb, Daxx, Ddit3, Ddx24, Ddx58, Ddx60, Dpp4, Eif2ak2, Emr1, Epsti1, Evi2a, Fcgr1, Fcgr4, Ftsjd2, Gcnt1, Gm11772, Gm14446, Gm15433, Gm16340, Gm20559, Gm2666, Gm7609, Gm9706, Gpnmb, Gpr15, H2-T10, H2-T9, Hck, Helz2, Herc6, Hsh2d, Hspa1b, Ifi202b, Ifi203, Ifi204, Ifi205, Ifi2712a, Ifi35, Ifi44, Ifi441, Ifit1, Ifit1, Ifit2, Ifit3, Ifitm3, I118, I17r, Inpp5b, Ins16, Irf7, Isg20, Klrk1, Lga1s3bp, Lga1s9, LOC100041903, LOC100044068, LOC100503923, LOC100505160, LOC100862473, LOC664787, Lpar6, Ly6cl, Ly6c2, Mb21d1, Mitd1, Mlk1, Mmp8, Mnda, Mnda1, Ms4a4c, Ms4a6d, Mx1, Naa20, Nceh1, Ncoa7, Ngp, Nlrc5, Nmral1, Nqo1, Nt5c3, Oas1a, Oas2, Oas3, Oas11, Oas12, Ogfr, Papd7, Parp10, Parp11, Parp12, Parp14, Phf11d, Pik3ap1, Pla2g7, Plec, Pnpt1, Ppm1k, Pydc4, Pyhin1, Ramp3, Rnf213, Rnf8, Rsad2, Rtp4, Samd91, Scin, Sdc3, Setdb2, Sgcb, Shisa5, Slco3a1, Slfn1, Slfn3, Slfn4, Slfn5, Slfn8, Slfn9, St3ga16, Tcstv3, Tdrd7, Tiparp, Tmem140, Tmeml84b, Tnfsf10, Torlaip2, Tor3a, Trafd1, Trim25, Trim30a, Trim30d, Trim34a, Trim34b, Tspo, Uba7, Ubr4, Usp18, Wnt10a, Xaf1, Xaf1, Zc3hav1, Zcchc2, Zfyve26, Znfx1, and Zufsp; xvi) determining the level of expression of at least one gene selected from the group consisting of Ccdc711, D13ERTD608E, Fads2, Gm2a, Ifi2711, Ighm, Klk1, Scd2, Siglech, and Tspan2; xvii) determining the level of expression of at least one gene selected from the group consisting of CC12, CYBASC3, CYP1B1, FCAR, HBEGF, ID1, IL1B, IL4I1, MSC, NQO1, PPP1R15A, PRDM1, SLC7A11, SRXN1, TIPARP, TMEM138, TXNRD1, and VEGF; xviii) determining the level of expression of at least one gene selected from the group consisting of BCL2, CACNA2D3, C13ORF18, C20ORF103, C5ORF13, CDCA7, DEPDC6, GATM, HAL, HSPA1A, HSPC049, LOC645919, LRMP, OAF, POU4F2, RASGRP2, RET, SERPINB2, SERPINB8, SPFH1, and TDRD7; xix) determining the level of expression of at least one gene selected from the group consisting of ABCC1, ABHD12, ABHD5, ACPP, ACSL1, ADFP, ADORA2B, ADORA3, AHRR, AKNA, AKR1C1, AKR1C2, AKR1C3, ALAS1, ALOX5AP, ANKRD57, ANXA2, APBB1IP, APRIN, ARHGAP20, ARHGEF3, ARRB2, ARRDC4, ASB2, ATF5, ATG7, ATP6V0B, ATP6V0C, ATP9A, ATP9B, AXL, AYTL1, BCL2A1, BCL3, BCL6, BHLHB2, BTG1, BTG2, BTG3, C10ORF22, C10ORF54, C10ORF56, C12ORF35, C13ORF31, C14ORF43, C15ORF39, C17ORF32, C19ORF58, C1ORF122, C1ORF144, C1ORF162, C1ORF21, C1ORF38, C3AR1, C5ORF20, C6ORF166, C9ORF16, C9ORF88, CALN1, CARD15, CCL2, CCL5, CCND3, CCNL1, CCR1, CD109, CD244, CD300A, CD40, CD44, CD83, CD9, CDCA4, CDK5RAP2, CDKN1A, CHST11, CIDEC, CKB, CLEC5A, CLEC7A, CMTM3, CPEB2, CPEB4, CSF1R, CSGLCA-T, CSGLCA-T, CSPG2, CSPG2, CTSB, CTSH, CUTL1, CXCL1, CXCL2, CXXC5, CYBASC3, CYBB, CYLD, CYLD, CYP1B1, DDB1, DGAT2, DKFZP68601327, DOC1, DOK2, DUSP6, EBI2, ECGF1, ECOP, EFHD2, EIF1, ELL2, ELOVL1, EMP2, EMR2, EPAS1, EPB41L3, EPB41L3, EXT1, F3, FADS3, FAM100B, FCAMR, FCAR, FGD3, FGD4, FGL2, FLJ20489, FLJ20701, FLJ90013, FLRT2, FPRL1, FTH1, FUCA1, GAS7, GCNT1, GNA15, GPR35, GPR68, GSR, GSR, H2A, HBEGF, HERPUD1, HIPK2, HIST2H2AA, HIVEP1, HMOX1, HNRPLL, HPCAL1, ID1, ID2, IER5, IFI30, IFNGR1, IFNGR2, IGFBP3, MORA, IL1B, IL1R1, IL1RN, IL1RN, IL21R, IL27RA, IL27RA, IL4I1, IL4R, IRF5, ITGB7, JDP2, JUN, JUNB, JUND, KCNN4, KIAA0247, KIAA0999, KIAA1505, KIAA1706, KIAA1913, KITLG, KLF13, KLF4, KLF6, KLHL18, LACTB, LAT, LHX2, LOC113179, LOC338758, LOC440934, L0054103, LOC644242 LOC648998 LOC650429, LOC650446, LOC651816, LOC653524, LOC653361, LOC653840, LOC653361, LOC653506, LOC653610, LOC653840, LOC653626, LPAAT-THETA, LPL, LPXN, LRG1, LRP10, MAFB, MAFF, MALT1, MAML2, MAP1LC3B, MARCKSL1, MBP, MCL1, ME1, METRNL, MGAT4A, MGC13379, MGLL, MMP2, MMP9, MOBKL2A, MSC, MST150, MTF1, MTUS1, MYH10, NAB1, NCF1, NCF2, NCF4, NEU1, NFE2L3, NFKB1, NFKB2, NFKBIA, NFKBIE, NFXL1, NINJ1, NOTCH1, NOTCH2NL, NPTX1, NQO1, NRP1, NRP2, NT5E, NUAK1, P2RY5, P2RY6, PACSIN2, PDCD6, PDK4, PDLIM4, PECAM1, PEX19, PGD, PHLDA1, PHLDA2, PIK3R5, PIR, PITPNA, PKM2, PLAU, PLAUR, PLEKHO1, PNKD, POPDC3, PPIF, PPP1R15A, PRDM1, PRKCA, PSCD4, PSCDBP, PSMD1, PTAFR, PTGS1, PTPN14, PTPRE, PTX3, QPRT, RAB13, RAB27B, RAB38, RAB6IP1, RAI17, RAP2B, RAPGEF1, RCN1, RELB, RGL1, RGS1, RGS2, RIN3, RIT1, RND3, RSNL2, RSPO3, RUNX3, SAMSN1, SAP30, SASH1, SAT1, SDC4, SEMA4C, SERPINE2, SERTAD1, SFRS7, SGK, SH3GL1, SH3TC1, SLAMF8, SLC15A3, SLC16A3, SLC20A1, SLC23A2, SLC25A14, SLC25A19, SLC25A20, SLC2A1, SLC2A6, SLC37A2, SLC39A8, SLC43A2, SLC45A3, SLC4A2, SLC4A5, SLC6A6, SLC7A11, SLC7A11, SLIC1, SMOX, SNAI3, SOD2, SPRY2, SPSB1, SQRDL, SQSTM1, SRXN1, SSH1, ST3GAL5, STAT1, STK40, TFAP2A, TFDP1, TFEB, TGIF, THBD, TIPARP, TMEM138, TMTC1, TNFAIP3, TNFAIP6, TNFAIP8L1, TNFRSF10D, TNFRSF1B, TNFRSF21, TNFSF13B, TNFSF7, TP53BP2, TRAF3, TRAF3IP2, TRIB1, TRIB3, TRIM16, TRIM16L, TRPA1, TRPS1, TRPS1, TSHZ3, TTLL4, TXNRD1, UBE2S, UGCG, ULBP2, UPP1, URP2, VASH1, VEGF, VSNL1, YRDC, ZBTB24, ZCCHC10, ZFAND5, ZFP36L1, ZNF366, ZNF516, and ZNF697; xx) determining the level of expression of at least one gene selected from the group consisting of ABHD14B, ACTN1, ACY1L2, ADA, ADD2, AFF1, AIG1, AK2, AKAP1, ALS2CR13, ANKRD45, ANKRD55, APPL, ARHGEF6, ATG16L2, ATP8B3, ATP8B4, ATPBD1C, B3GNT7, BCL11A, BCL2, BMP8B, BRE, BSPRY, BTBD14A, C13ORF18, C13ORF18, C14ORF106, C15ORF41, C16ORF73, C1ORF121, C1ORF63, C1QBP, C1S, C20ORF103, C20ORF112, C20ORF12, C3ORF14, C5ORF13, C6ORF147, C7ORF24, C9ORF103, CABC1, CACNA2D3, CACYBP, CALCOCO2, CAMSAP1L1, CAMSAP1L1, CAT, CAV1, CDCA7, CERKL, CHST12, CHST5, CITED4, CLINT1, CLSTN2, CLTCL1, CNTN4, COL4A1, COL8A2, CUGBP2, CXORF21, DAB1, DENND4A, DEPDC6, DHRS9, DMRT2, DUT, EIF4A2, ESD, FLJ12078, FLJ20152, FLJ23861, FLJ36166, FOXP1, GATM, GGA2, GOLGA1, GOLGA8C, GOLGA8D, GOLGA8E, GOLGA8F, GOLGA8G, GPD1L, GPR18, HADH, HAL, HDAC9, HGF, HIG2, HISPPD1, HNRPA3, HNRPH3, HOXB3, HSPA1A, HSPA4L, HSPB1, HSPC049, ID2, ID2B, IDH1, IDH1, IHPK2, IRX3, ITGA4, KBTBD11, KCNN2, KIAA0960, KLF10, LARS, LGR4, LIMA1, LIX1L, LOC129285, LOC148203, LOC197322, LOC203274, LOC220594, LOC254559, LOC284702, LOC285084, LOC285758, LOC340061, LOC340061, LOC388189, LOC474170, LOC643458, LOC645919, LOC646456, LOC90835 LONRF1, LRMP, LYST, MACF1, MDH1, METTL7B, METTL8, MICAL1, MLSTD1, MNDA, MRPL24, MS4A3, MS4A4A, MS4A6A, MS4A7, MSRB3, MT1E, MT1H, MT1M, MTBP, MTHFD1, MTL5, MTR, MUC19, MUM1, MYADM, NAPSB, NAPSB, NAT11, NOC2L, NPAL3, OAF, OCRL, OMA1, OSBPL1A, OXCT2, PDCD4, PHACTR3, PHYH, PIGM, PIWIL4, PNMA6A, POU4F2, PRKAB2, PRLR, PSAT1, PSAT1, PTGER3, PTPLAD2, RABGAP1L, RAD17, RASGRP2, RBKS, RET, RNASEH2B, RNASET2, SELPLG, SERPINB10, SERPINB2, SERPINB8, SERPINI2, SKAP2, SLAIN1, SLC16A4, SLC22A15, SLC22A16, SLC40A1, SMARCA2, SNAPC3, SNX10, SPFH1, SPTBN1, ST3GAL3, STAR, STRBP, SYNPO2, TADA1L, TCFL5, TDRD7, THTPA, TIFA, TLE1, TMEM14A TOP2B, TPD52, TPM1, TRAF3IP3, TSPAN2, TTC9C, UBE2B, UBP1, UHRF2, VLDLR, VPS35, WASF1, WDFY1, WDR49, WDR68, WHDC1L1, WHDC1L2, ZBTB33, ZBTB44, ZF, ZNF207, ZNF519, ZNF658, and ZNF92; xxi) determining the level of expression of at least one gene selected from the group consisting of CCL2, CCL5, CXCL10, IL1RN, and MMP9; xxii) determining the level of expression of at least one gene selected from the group consisting of CCL5, CXCL10, and MMP9; xxiii) determining the level of expression of at least one gene selected from the group consisting of IL10 and CCL2; xxiv) determining the level of expression of at least one gene selected from the group consisting of IFNg, TNF, CCL3, CXCL8, and IL-10; xxv) determining the level of expression of at least one gene selected from the group consisting of MMP9, CCL2, CCL5, CXCL1, and IL1B; xxvi) determining the level of expression of at least one gene selected from the group consisting of MMP9, CXCL10, CCL2, and CCL5; xxix) determining the level of expression of at least one gene selected from the group consisting of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, and MIF; or xxx) determining the level of expression of at least one gene selected from the group consisting of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, MIF, IL16, IL6, CCL25, IL2, CCL19, CXCL2, CXCL9, and CXCL5,
   thereby characterizing the glatiramer acetate related drug substance or drug product of step a).

This application claims the benefit of U.S. Provisional Application No. 62/162,308, filed May 15, 2015, U.S. Provisional Application No. 62/134,245, filed Mar. 17, 2015, U.S. Provisional Application No. 62/078,369, filed Nov. 11, 2014, U.S. Provisional Application No. 62/047,437, filed Sep. 8, 2014, U.S. Provisional Application No. 62/025,953, filed Jul. 17, 2014, U.S. Provisional Application No. 62/020,358, filed Jul. 2, 2014, and U.S. Provisional Application No. 62/019,857, filed Jul. 1, 2014, the contents of which are hereby incorporated by reference.

Throughout this application various publications are referenced by numerical identifiers in parentheses. Full citations of these references can be found following the Examples. The disclosures of these publications in their entireties are hereby incorporated by reference into this application in order to more fully describe the state of the art to which this invention pertains.

Multiple sclerosis (MS) is a chronic, debilitating autoimmune disease of the central nervous system (CNS) with either relapsing-remitting (RR) or progressive course leading to neurologic deterioration and disability. At time of initial diagnosis, RRMS is the most common form of the disease (1) which is characterized by unpredictable acute episodes of neurological dysfunction (relapses), followed by variable recovery and periods of clinical stability. The vast majority of RRMS patients eventually develop secondary progressive (SP) disease with or without superimposed relapses. Around 15% of patients develop a sustained deterioration of their neurological function from the beginning; this form is called primary progressive (PP) MS. Patients who have experienced a single clinical event (Clinically Isolated Syndrome or “CIS”) and who show lesion dissemination on subsequent magnetic resonance imaging (MRI) scans according to McDonald's criteria, are also considered as having relapsing MS.(2)

With a prevalence that varies considerably around the world, MS is the most common cause of chronic neurological disability in young adults.(3, 4) Anderson et al. estimated that there were about 350,000 physician-diagnosed patients with MS in the United States in 1990 (approx. 140 per 100,000 population).(5) It is estimated that about 2.5 million individuals are affected worldwide.(6) In general, there has been a trend toward an increasing prevalence and incidence of MS worldwide, but the reasons for this trend are not fully understood.(5)

Current therapeutic approaches consist of i) symptomatic treatment ii) treatment of acute relapses with corticosteroids and iii) treatment aimed to modify the course of the disease. Currently approved therapies target the inflammatory processes of the disease. Most of them are considered to act as immunomodulators but their mechanisms of action have not been completely elucidated. Immunosuppressants or cytotoxic agents are also used in some patients after failure of conventional therapies. Several medications have been approved and clinically ascertained as efficacious for the treatment of RR-MS; including BETASERON®, AVONEX® and REBIF®, which are derivatives of the cytokine interferon beta (IFNB), whose mechanism of action in MS is generally attributed to its immunomodulatory effects, antagonizing pro-inflammatory reactions and inducing suppressor cells.(7) Other approved drugs for the treatment of MS include Mitoxantrone and Natalizumab.

Copaxone® (Teva Pharmaceutical Industries Ltd.) is a glatiramer acetate drug product approved for treatment of patients with relapsing-remitting multiple sclerosis (RRMS) and clinically isolated syndrome (CIS) (8). Glatiramer acetate drug substance (GA), the active substance of Copaxone®, is a complex mixture of polypeptides and is the first member of the glatiramoid class; i.e., a complex mixture of synthetic polypeptides of varying sizes assembled from four naturally occurring amino acids: L-glutamic acid, L-alanine, L-lysine, and L-tyrosine, in a defined molar ratio (9).

GA elicits anti-inflammatory as well as neuroprotective effects in various animal models of chronic inflammatory and neurodegenerative diseases (10-14) and has been shown to be safe and effective in reducing relapses and delaying neurologic disability in MS patients following long-term treatment (15).

The mechanisms underlying GA therapeutic activity are not fully elucidated, but GA activity on immune cells has been well demonstrated. GA appears to act as an altered peptide ligand (APL) of encephalitogenic epitopes within myelin basic protein (MBP) (16) and demonstrates cross-reactivity with MBP at the humoral and cellular levels (17-23). The unique antigenic sequences of the GA polypeptide mixture compete with myelin antigens for binding to MHC class II molecules on antigen presenting cells. (APCs) and presentation to the T cell receptor (TCR), resulting in the induction of anergy or deletion of autoreactive MBP-reactive T cells and proliferation of GA-reactive T cells. At initiation of Copaxone® treatment, GA-reactive CD4+ T-cell lines from MS patients secrete both pro-inflammatory T helper type 1 (Th1) and anti-inflammatory Th2 cytokines (21, 24), but continued exposure to Copaxone® induces a shift in GA-reactive T cells toward the Th2 phenotype (21, 23, 25-28).

Copaxone® also increases the number and suppressive capacity of CD4+CD25+FOXP3+ regulatory T cells, which are functionally impaired in MS patients (29-31). Furthermore, treatment leads to antigen-nonspecific modulation of APC function. Copaxone® treatment promotes development of anti-inflammatory type II monocytes characterized by an increase in interleukin (IL)-10 and transforming growth factor-beta (TGF-β) and decreased production of IL-12 and tumor necrosis factor (TNF) (32).

### SUMMARY OF THE INVENTION

The present invention provides a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) i) determining the level of expression of at least one gene selected from the group consisting of ABCF2, ABI2, ACP6, AFG3L2, ALMS1, ARPC4, CALMS, CCDC64, CD84, CDC6, CHAF1A, CLU, COX11, DLGAP1, DTX4, FAM49B, FHL1, FNTB, GYPC, HFE, IL10, LPHN1, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, and ZFAND6 (hereinafter Gene Group 1); ii) determining the level of expression of at least one gene selected from the group consisting of BIRC3, CCL24, CCR1, CISH, CSF1R, CX3CR1, CXCL10, HSPD1, ICAM1, IL1B, IFNGR1, IL27, IL2RG, IL7R, IL1RN, MMP1, MMP9, MMP14, PGRMC1, PRDM1, CARD15, CCL2, CCL5, CD14, IL10, THBD, and NFKBIA (hereinafter Gene Group 2), wherein if IL1B, IL10, or MMP9 is the at least one gene selected in part (c) (ii), then selecting at least a second different gene from the group other than IL1B, IL10, or MMP9; iii) determining the level of expression of at least one gene selected from the group consisting of C13ORF31, C14ORF10, C1ORF51, C1ORF63, CBR4, CB36, CD9, COL6A1, DAB2, GATA2, KIAA0907, LOC100506233, MCM6, MMP1, MS4A4A, MTSS1, PCMTD1, STK4, STX7, TAF15, TARP, TIA1, TMF1, TRGC2, TXNDC11, and ZCCHC7 (hereinafter Gene Group 3); iv) determining the level of expression of at least one gene selected from the group consisting of ANXA1, ARRB2, BEAN, BIN1, C1ORF63, CD44, CD9, CFP, COL6A1, CRIP2, EPB41, Fam119a, FGR, FOXO3B, HSD11B1, HSPD1P6, LOC387790, MPEG1, MYB, OLIG1, PLD1, PPP4R2, PRDM1, RBM6, SNX27, SOD2, STATH, TARP, TREM1, TRGC2, UBN2, and ZCCHC7 (hereinafter Gene Group 4); v) determining the level of expression of at least one gene selected from the group consisting of ADAM9, ADAMDEC1, AKR1C2, ANXA2, ANXA2P2, ARHGAP18, ARHGAP18, ARL6IP5, ARL6IP5, ATP2C1, BID, BIRC3, BTG1, CARD15, C1ORF21, C13ORF31, C5ORF13, C5ORF32, C9ORF130, CAST, CCL2, CCL5, CD14, CD300A, CD36, CD40, CD55, CD9, CENTA2, CHST11, COL6A1, CRYBB2, CXCL10, CYLD, DAB2, EBI3, EBI2, ECOP, EGF, FABP4, FXYD2, GHRL, GIMAP8, GLIPR1, GOS2, HMGB2, HNRPLL, ICAM1, ICAM2, IFIH1, IFNGR1, IL10, IL1ORA, IL411, INADL, ISG20, ITGB5, KIAA1505, KYNU, LACTB, LOC54103, LOC388344, LOC652751, LPAAT-THETA, LPXN, MAFB, MALT1, MFI2, MGC5618, MGLL, MITF, MLF1, MMP1, MMP9, MPEG1, MTSS1, MXD1, NT5E, NFKBIE, NFKBIA, NFE2L3, NFE2L3, OSBPL11, P2RX4, P2RY5, PLEKHO1, POPDC3, PLAUR, PRDM1, PSCDBP, PTX3, RAB27B, RCSD1, RPL13, SGIP1, SLC39A8, SNORD68, SRPX2, SRA1, SLIC1, SLAMF8, SLIC1, SOD2, STATH, STEAP1, SYNJ2, SYNJ2, TATDN3, TGM5, THBD, TNFAIP3, TNFAIP6, TNFRSF9, TNFSF13B, TPSAB1, TPSB2, TREM1, TXNL2, VPS33A, and VSNL1 (hereinafter Gene Group 5); vi) determining the level of expression of at least one gene selected from the group consisting of ACTN4, BTBD14A, C14ORF10, CISH, CLK1, CRLF3, FAM62A, FBXO45, GAPDHS, HDAC4, HIC2, HNRPD, HSPD1, LOC648342, MYB, NAPB, OXCT2, SERPINB2, SFRS14, SPFH1, STT3B, WDFY1, ZNF250, and ZNF566 (hereinafter Gene Group 6); vii) determining the level of expression of at least one gene selected from the group consisting of A2M, ABCB1, ABCC3, ABHD2, ACPP, ADAMDEC1, ADFP, ADORA2B, ADORA3, AHNAK, ALCAM, ANKH, ANKRD57, ANXA2, ANXA2P2, APBB1IP, AQP1, ARHGAP18, ARHGAP20, ARHGEF3, ARID5B, ARMC9, ATF5, ATP1B1, ATP6V0D2, ATP9A, ATP10A, AYTL1, BCL2A1, BCL6, C3AR1, C13ORD31, C9ORF88, C9ORF89, C1ORF21, C1ORF21, C10ORF95, C13ORF31, C21ORF7, CARD12, CARD15, CCDC83, CCL5, CCL24, CCND1, CCR1, CD9, CD36, CD52, CD86, CD109, CD180, CD244, CDK5RAP2, CDKN1A, CENTA2, CKB, CKLF, CLEC7A, CNIH3, COL6A1, COL22A1, CRIP2, CSF1R, CSPG4, CTSL, CTSLL3, CX3CR1, CXCR7, CYBB, CYP1B1, DAB2, DAPP1, DDIT4L, DIXDC1, DOCK4, DOK2, DKFZP56400823, DKFZP68601327, EBI2, EMP1, EMR2, ENPP2, EPAS1, EPS8, EPSTI1, EVL, FABP4, FADS3, FAM26B, FGD2, FGD2, FGD4, FGL2, FN1, FTH1, GBP2, GBP3, GBP5, GCNT1, GDPD1, GNDL, GNLY, GLIPR1, GLIS3, GPC1, GPR35, H2A/R, HAVCR2, HMCN1, HIPK2, HIST2H2AA, HIVEP1, HMOX1, HSPB7, ICAM1, ID2, ID2B, IFI30, IFI44, IFNGR1, IGFBP3, IL2RG, IL4I1, IL10RA, IL27RA, IL7R, IL10, INA, IRF7, ITGB5, ITGB7, KIAA1505, KIAA1706, KMO, LBH, LFNG, LILRB1, LILRB2, LMNA, LOC51334, LOC201895, LOC284262, LOC51334, LOC643424, LOC643834, LOC643847, LOC644242, LOC645238, LOC650429, LOC650446, LOC652543, LOC653610, LOC653754, LPAAT-THETA, LPXN, MAF, MAFB, MAML2, MAML3, MARCH1, MCOLN3, MDGA1, ME1, MFI1, MFI2, MGC45491, MGLL, MITF, MMP1, MMP2, MMP9, MMP14, MMP19, MTMR11, MTSS1, MTSU1, NGEF, NME7, NPTX1, NRCAM, NRP1, NRP2, NT5E, OAS1, OLR1, P2RY5, P2RY14, PALLD, PAPSS2, PAQP5, PCDHGA1, PCDHGA2, PCDHGA3, PCDHGA4, PCDHGA5, PCDHGA6, PCDHGA7, PCDHGA8, PCDHGA9, PCDHGA10, PCDHGA11, PCDHGA12, PCDHGB1, PCDHGB2, PCDHGB3, PCDHGB4, PCDHGB5, PCDHGB7, PCDHGC3, PCDHGC4, PCDHGC5, PDK4, PDLIM7, PFKFB4, PGA5, PDLIM4, PHLDA1, PLA2G4A, PLEKHA7, PLEKHO1, POPDC3, PRDM1, PRSS23, PSCDBP, PSD3, PTAFR, PTGS1, PTPRO, PTRF, PTX3, RAB27B, RAB38, RAB7B, RAPH1, RASGRF1, RGL1, RGS13, RHBDF1, RIN2, S100A2, SART2, SERPINE2, SERTAD1, SETBP1, SGIP1, SH3TC1, SKIL, SLA, SLAMF7, SLAMF8, SLC6AS, SLC7A11, SLC12A6, SLC37A2, SLC41A2, SLC38A6, SLC43A2, SNAI3, ST3GAL5, STATH, STEAP1, SUCNR1, SYTL1, TBXAS1, TCF4, TFAP2A, THBD, TLR4, TM7SF4, TMEM39A, TMEM158, TNCRNA, TNFSF13B, TNFRSF21, TREM1, TRIM22, TRPA1, TRPM8, TRPS1, TUBB2A, UBXD5, UGCG, UPP1, VASH1, VEGF, VSNL1, and ZFP36L1 (hereinafter Gene Group 7); viii) determining the level of expression of at least one gene selected from the group consisting of ABCG1, ADAMTS1, ANKRD41, ANXA3, APCDD1, BCL2, BCL11A, BMP8B, C1ORF71, C1ORF76, C1ORD121, C12ORF24, C16ORF73, C16ORF74, C20ORD27, C20ORF103, C20ORF112, CACNA2D3, CAMTA1, CAV1, CCDC85B, CDCA7L, CEBPD, CKAP4, CNTN4, COL8A2, CSPG5, CXCR4, DCUN1D4, DEPDC6, DMRT2, DUSP2, DZIP1, EBF3, EGR4, FAM117A, FKBP4, FL135848, FLOT2, GFI1, GMDS, GPR18, HAL, HNF4G, HSPC049, IL17D, IRX3, KBTBD11, KCNQ4, KCTD15, KIAA0146, KIAA0984, KIAA1026, KIAA1553, KLHL23, LGR4, LOC201164, LOC284454, LOC387763, LOC642083, LOC648232, MGC2408, MICAL1, MID1IP1, MSRB3, MUC19, NAPSB, NR1D2, PCDH8, PDE4B, PDGFD, PER2, PHF15, PKP2, PLK2, OAF, OSBPL1A, OSR2, OXCT2, PGM1, PMAIP1, PNMA6A, POU4F2, PSAT1, RAB33A, RASGRP2, RBM38, RET, RFTN1, SERPINE2, SERPINB10, SLAIN1, SLC1A3, SLC16A1, SLC19A1, SLC27A2, SLC29A1, SLC39A14, SLCO4A1, SNF1LK, SOX12, SPFH1, SPRY1, STEAP3, SYDE2, SYNPO2, TARP, TEAD4, TDRD7, TMEM67, TPD52, TRGC2, TRGV2, TRGV9, TRIB3, TSPAN2, TUBA1, VIT, WDR49, WNT3, WT1, and YES1 (hereinafter Gene Group 8); ix) determining the level of expression of at least one gene selected from the group consisting of AHRR, CCDC36, CYP1B1, DOC1, EPB41L3, GAS7, GPR68, NPTX1, PDCD6, and TIPARP (hereinafter Gene Group 9); x) determining the level of expression of at least one gene selected from the group consisting of ADRB2, COTL1, LOC285758, LOC644137, MALAT1, PRG1, RNF43, SAT1, THAP5, TIMP3, and TSC22D1 (hereinafter Gene Group 10); xi) determining the level of expression of at least one gene selected from the group consisting of AW011738, Bst2, Daxx, Gm16340, Hck, Herc6, Ifi202b, Ifi203, Ifi204, Ifi44, Ifi441, Ifit2, Inpp5 b, LOC100044068, LOC100862473, Mx1, Oas11, Phf11d, Oyhin1, Sdc3, Setdb2, Tor3a, Usp18, and Zcchc2 (hereinafter Gene Group 11); xii) determining the level of expression of at least one gene selected from the group consisting of Ccdc711, D13ERTD608E, Fads2, Gm2a, Ifi2711, Ighm, Klk1, Scd2, Siglech, and Tspan2 (hereinafter Gene Group 12); xiii) determining the level of expression of at least one gene selected from the group consisting of Ahrr, AI607873, Atp10a, AW011738, Casp44, Cxc13, Gm9706, Ifi202b, Ifit2, Ifitm6, I118, Lcn2, LOC100044068, Ms4a6d, Mx1, Papd7, Rsad2, Slfn3, Slfn4, Tdrd7, Tiparp, and Zcchc2 (hereinafter Gene Group 13); xiv) determining the level of expression of at least one gene selected from the group consisting of Aldoc, Casp6, Ccdc711, Cox7a1, Egln3, Fam162a, Gfi1, Gpi1, Grhpr, Ifi2711, Ighm, Kcnq5, Klhdc2, Pgk1, Pkm, Tpi1, and Trappc6a (hereinafter Gene Group 14); xv) determining the level of expression of at least one gene selected from the group consisting of 1600014C10Rik, 2810474O19Rik, 6720475J19Rik, Adam8, Adar, Agrn, Ahrr, AI607873, Amigo2, Ankfy1, Apobec1, Arf4, Asb2, Ascc3, Atp10a, Atp8b4, AW011738, B4galt5, BC147527, Bst2, Casp4, Chic1, Cmpk2, Csprs, Cxc13, Cybb, Daxx, Ddit3, Ddx24, Ddx58, Ddx60, Dpp4, Eif2ak2, Emr1, Epsti1, Evi2a, Fcgr1, Fcgr4, Ftsjd2, Gcnt1, Gm11772, Gm14446, Gm15433, Gm16340, Gm20559, Gm2666, Gm7609, Gm9706, Gpnmb, Gpr15, H2-T10, H2-T9, Hck, Helz2, Herc6, Hsh2d, Hspa1b, Ifi202b, Ifi203, Ifi204, Ifi205, Ifi2712a, Ifi35, Ifi44, Ifi441, Ifih1, Ifit1, Ifit2, Ifit3, Ifitm3, I118, I17r, Inpp5b, Ins16, Irf7, Isg20, Klrk1, Lgals3 bp, Lgals9, LOC100041903, LOC100044068, LOC100503923, LOC100505160, LOC100862473, LOC664787, Lpar6, Ly6c1, Ly6c2, Mb21d1, Mitd1, Mlk1, Mmp8, Mnda, Mnda1, Ms4a4c, Ms4a6d, Mx1, Naa20, Nceh1, Ncoa7, Ngp, Nlrc5, Nmral1, Nqo1, Nt5c3, Oas1a, Oas2, Oas3, Oas11, Oas12, Ogfr, Papd7, Parp10, Parp11, Parp12, Parp14, Phf11d, Pik3ap1, Pla2g7, Plec, Pnpt1, Ppm1k, Pydc4, Pyhin1, Ramp3, Rnf213, Rnf8, Rsad2, Rtp4, Samd9l, Scin, Sdc3, Setdb2, Sgcb, Shisa5, Slco3a1, Slfn1, Slfn3, Slfn4, Slfn5, Slfn8, Slfn9, St3gal6, Tcstv3, Tdrd7, Tiparp, Tmem140, Tmem184b, Tnfsf10, Torlaip2, Tor3a, Trafd1, Trim25, Trim30a, Trim30d, Trim34a, Trim34b, Tspo, Uba7, Ubr4, Usp18, Wnt10a, Xaf1, Xaf1, Zc3hav1, Zcchc2, Zfyve26, Znfx1, and Zufsp (hereinafter Gene Group 15); xvi) determining the level of expression of at least one gene selected from the group consisting of Ccdc711, D13ERTD608E, Fads2, Gm2a, Ifi2711, Ighm, Klk1, Scd2, Siglech, and Tspan2 (hereinafter Gene Group 16); xvii) determining the level of expression of at least one gene selected from the group consisting of CC12, CYBASC3, CYP1B1, FCAR, HBEGF, ID1, IL1B, IL4I1, MSC, NQO1, PPP1R15A, PRDM1, SLC7A11, SRXN1, TIPARP, TMEM138, TXNRD1, and VEGF (hereinafter Gene Group 17); xviii) determining the level of expression of at least one gene selected from the group consisting of BCL2, CACNA2D3, C13ORF18, C20ORF103, C5ORF13, CDCA7, DEPDC6, GATM, HAL, HSPA1A, HSPC049, LOC645919, LRMP, OAF, POU4F2, RASGRP2, RET, SERPINB2, SERPINB8, SPFH1, and TDRD7 (hereinafter Gene Group 18); xix) determining the level of expression of at least one gene selected from the group consisting of ABCC1, ABHD12, ABHD5, ACPP, ACSL1, ADFP, ADORA2B, ADORA3, AHRR, AKNA, AKR1C1, AKR1C2, AKR1C3, ALAS1, ALOX5AP, ANKRD57, ANXA2, APBB1IP, APRIN, ARHGAP20, ARHGEF3, ARRB2, ARRDC4, ASB2, ATF5, ATG7, ATP6V0B, ATP6V0C, ATP9A, ATP9B, AXL, AYTL1, BCL2A1, BCL3, BCL6, BHLHB2, BTG1, BTG2, BTG3, C10ORF22, C10ORF54, C10ORF56, C12ORF35, C13ORF31, C14ORF43, C15ORF39, C17ORF32, C19ORF58, C1ORF122, C1ORF144, C1ORF162, C1ORF21, C1ORF38, C3AR1, C5ORF20, C6ORF166, C9ORF16, C9ORF88, CALN1, CARD15, CCL2, CCL5, CCND3, CCNL1, CCR1, CD109, CD244, CD300A, CD40, CD44, CD83, CD9, CDCA4, CDK5RAP2, CDKN1A, CHST11, CIDEC, CKB, CLEC5A, CLEC7A, CMTM3, CPEB2, CPEB4, CSF1R, CSGLCA-T, CSGLCA-T, CSPG2, CSPG2, CTSB, CTSH, CUTL1, CXCL1, CXCL2, CXXC5, CYBASC3, CYBB, CYLD, CYLD, CYP1B1, DDB1, DGAT2, DKFZP68601327, DOC1, DOK2, DUSP6, EBI2, ECGF1, ECOP, EFHD2, EIF1, ELL2, ELOVL1, EMP2, EMR2, EPAS1, EPB41L3, EPB41L3, EXT1, F3, FADS3, FAM100B, FCAMR, FCAR, FGD3, FGD4, FGL2, FLJ20489, FLJ20701, FLJ90013, FLRT2, FPRL1, FTH1, FUCA1, GAS7, GCNT1, GNA15, GPR35, GPR68, GSR, GSR, H2A, HBEGF, HERPUD1, HIPK2, HIST2H2AA, HIVEP1, HMOX1, HNRPLL, HPCAL1, ID1, ID2, IER5, IFI30, IFNGR1, IFNGR2, IGFBP3, IL1ORA, IL1B, IL1R1, IL1RN, IL1RN, IL21R, IL27RA, IL27RA, IL411, IL4R, IRF5, ITGB7, JDP2, JUN, JUNB, JUND, KCNN4, KIAA0247, KIAA0999, KIAA1505, KIAA1706, KIAA1913, KITLG, KLF13, KLF4, KLF6, KLHL18, LACTB, LAT, LHX2, LOC113179, LOC338758, LOC440934, LOC54103, LOC644242, LOC648998, LOC650429, LOC650446, LOC651816, LOC653524, LOC653361, LOC653840, LOC653361, LOC653506, LOC653610, LOC653840, LOC653626, LPAAT-THETA, LPL, LPXN, LRG1, LRP10, MAFB, MAFF, MALT1, MAML2, MAP1LC3B, MARCKSL1, MBP, MCL1, ME1, METRNL, MGAT4A, MGC13379, MGLL, MMP2, MMP9, MOBKL2A, MSC, MST150, MTF1, MTUS1, MYH10, NAB1, NCF1, NCF2, NCF4, NEU1, NFE2L3, NFKB1, NFKB2, NFKBIA, NFKBIE, NFXL1, NINJ1, NOTCH1, NOTCH2NL, NPTX1, NQO1, NRP1, NRP2, NT5E, NUAK1, P2RY5, P2RY6, PACSIN2, PDCD6, PDK4, PDLIM4, PECAM1, PEX19, PGD, PHLDA1, PHLDA2, PIK3R5, PIR, PITPNA, PKM2, PLAU, PLAUR, PLEKHO1, PNKD, POPDC3, PPIF, PPP1R15A, PRDM1, PRKCA, PSCD4, PSCDBP, PSMD1, PTAFR, PTGS1, PTPN14, PTPRE, PTX3, QPRT, RAB13, RAB27B, RAB38, RAB6IP1, RAI17, RAP2B, RAPGEF1, RCN1, RELB, RGL1, RGS1, RGS2, RIN3, RIT1, RND3, RSNL2, RSPO3, RUNX3, SAMSN1, SAP30, SASH1, SAT1, SDC4, SEMA4C, SERPINE2, SERTAD1, SFRS7, SGK, SH3GL1, SH3TC1, SLAMF8, SLC15A3, SLC16A3, SLC20A1, SLC23A2, SLC25A14, SLC25A19, SLC25A20, SLC2A1, SLC2A6, SLC37A2, SLC39A8, SLC43A2, SLC45A3, SLC4A2, SLC4A5, SLC6A6, SLC7A11, SLC7A11, SLIC1, SMOX, SNAI3, SOD2, SPRY2, SPSB1, SQRDL, SQSTM1, SRXN1, SSH1, ST3GAL5, STAT1, STK40, TFAP2A, TFDP1, TFEB, TGIF, THBD, TIPARP, TMEM138, TMTC1, TNFAIP3, TNFAIP6, TNFAIP8L1, TNFRSF10D, TNFRSF1B, TNFRSF21, TNFSF13B, TNFSF7, TP53BP2, TRAF3, TRAF3IP2, TRIB1, TRIB3, TRIM16, TRIM16L, TRPA1, TRPS1, TRPS1, T5HZ3, TTLL4, TXNRD1, UBE2S, UGCG, ULBP2, UPP1, URP2, VASH1, VEGF, VSNL1, YRDC, ZBTB24, ZCCHC10, ZFAND5, ZFP36L1, ZNF366, ZNF516, and ZNF697 (hereinafter Gene Group 19); xx) determining the level of expression of at least one gene selected from the group consisting of ABHD14B, ACTN1, ACY1L2, ADA, ADD2, AFF1, AIG1, AK2, AKAP1, ALS2CR13, ANKRD45, ANKRD55, APPL, ARHGEF6, ATG16L2, ATP8B3, ATP8B4, ATPBD1C, B3GNT7, BCL11A, BCL2, BMP8B, BRE, BSPRY, BTBD14A, C13ORF18, C13ORF18, C14ORF106, C15ORF41, C16ORF73, C1ORF121, C1ORF63, C1QBP, C1S, C20ORF103, C20ORF112, C20ORF12, C3ORF14, C5ORF13, C6ORF147, C7ORF24, C9ORF103, CABC1, CACNA2D3, CACYBP, CALCOCO2, CAMSAP1L1, CAMSAP1L1, CAT, CAV1, CDCA7, CERKL, CHST12, CHST5, CITED4, CLINT1, CLSTN2, CLTCL1, CNTN4, COL4A1, COL8A2, CUGBP2, CXORF21, DAB1, DENND4A, DEPDC6, DHRS9, DMRT2, DUT, EIF4A2, ESD, FLJ12078, FLJ20152, FLJ23861, FLJ36166, FOXP1, GATM, GGA2, GOLGA1, GOLGA8C, GOLGA8D, GOLGA8E, GOLGA8F, GOLGA8G, GPD1L, GPR18, HADH, HAL, HDAC9, HGF, HIG2, HISPPD1, HNRPA3, HNRPH3, HOXB3, HSPA1A, HSPA4L, HSPB1, HSPC049, ID2, ID2B, IDH1, IDH1, IHPK2, IRX3, ITGA4, KBTBD11, KCNN2, KIAA0960, KLF10, LARS, LGR4, LIMA1, LIX1L, LOC129285, LOC148203, LOC197322, LOC203274, LOC220594, LOC254559, LOC284702, LOC285084, LOC285758, LOC340061, LOC340061, LOC388189, LOC474170, LOC643458, LOC645919, LOC646456, LOC90835 LONRF1, LRMP, LYST, MACF1, MDH1, METTL7B, METTL8, MICAL1, MLSTD1, MNDA, MRPL24, MS4A3, MS4A4A, MS4A6A, MS4A7, MSRB3, MT1E, MT1H, MT1M, MTBP, MTHFD1, MTL5, MTR, MUC19, MUM1, MYADM, NAPSB, NAPSB, NAT11, NOC2L, NPAL3, OAF, OCRL, OMA1, OSBPL1A, OXCT2, PDCD4, PHACTR3, PHYH, PIGM, PIWIL4, PNMA6A, POU4F2, PRKAB2, PRLR, PSAT1, PSAT1, PTGER3, PTPLAD2, RABGAP1L, RAD17, RASGRP2, RBKS, RET, RNASEH2B, RNASET2, SELPLG, SERPINB10, SERPINB2, SERPINB8, SERPINI2, SKAP2, SLAIN1, SLC16A4, SLC22A15, SLC22A16, SLC40A1, SMARCA2, SNAPC3, SNX10, SPFH1, SPTBN1, ST3GAL3, STAR, STRBP, SYNPO2, TADA1L, TCFL5, TDRD7, THTPA, TIFA, TLE1, TMEM14A TOP2B, TPD52, TPM1, TRAF3IP3, TSPAN2, TTC9C, UBE2B, UBP1, UHRF2, VLDLR, VPS35, WASF1, WDFY1, WDR49, WDR68, WHDC1L1, WHDC1L2, ZBTB33, ZBTB44, ZF, ZNF207, ZNF519, ZNF658, and ZNF92 (hereinafter Gene Group 20); xxi) determining the level of expression of at least one gene selected from the group consisting of CCL2, CCL5, CXCL10, IL1RN, and MMP9 (hereinafter Gene Group 21); xxii) determining the level of expression of at least one gene selected from the group consisting of CCL5, CXCL10, and MMP9 (hereinafter Gene Group 22); xxiii) determining the level of expression of at least one gene selected from the group consisting of IL10 and CCL2 (hereinafter Gene Group 23); xxiv) determining the level of expression of at least one gene selected from the group consisting of IFNg, TNF, CCL3, CXCL8, and IL-10 (hereinafter Gene Group 24); xxv) determining the level of expression of at least one gene selected from the group consisting of MMP9, CCL2, CCL5, CXCL1, and IL1B (hereinafter Gene Group 25); xxvi) determining the level of expression of at least one gene selected from the group consisting of MMP9, CXCL10, CCL2, and CCL5 (hereinafter Gene Group 26); xxix) determining the level of expression of at least one gene selected from the group consisting of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, and MIF (hereinafter Gene Group 29); or xxx) determining the level of expression of at least one gene selected from the group consisting of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, MIF, IL16, IL6, CCL25, IL2, CCL19, CXCL2, CXCL9, and CXCL5 (hereinafter Gene Group 30),
   thereby characterizing the glatiramer acetate related drug substance or drug product of step a).

The present invention also provides a process for discriminating between glatiramer acetate related drug substances or drug products comprising the steps of:
a) characterizing two or more glatiramer acetate related drug substances or drug products according to the process of the claimed invention to obtain characteristics of each of the glatiramer acetate related drug substances or drug products; and
b) comparing the characteristics of the glatiramer acetate related drug substances or drug products obtained in step a), thereby discriminating between glatiramer acetate related drug substances or drug products.

The present invention also provides a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) xxii) determining the protein level expression of at least one protein selected from the group consisting of RANTES, IP-10, and MMP-9 (hereinafter Protein Group A); xxiii) determining the protein level expression of at least one protein selected from the group consisting of IL10 and MCP-1 (hereinafter Protein Group B); xxiv) determining the protein level expression of at least one protein selected from the group consisting of IFNg, TNFa, MIP-1a, IL-8, and IL-10 (hereinafter Protein Group C); xxv) determining the protein level expression of at least one protein selected from the group consisting of MMP-9, MCP-1, RANTES, Gro-a, and IL-1b (hereinafter Protein Group D); xxvi) determining the protein level expression of at least one protein selected from the group consisting of MMP-9, IP-10, MCP-1, and RANTES (hereinafter Protein Group E); xxvii) determining the protein level expression of at least one protein selected from the group consisting of MIP-1a, MMP-9, MDC, CCL24, CX3CL1, MIP-3a, MCP-1, TNF-α, IL-8, MCP-4, RANTES, IL-1b, MCP-2, IL-10, I-TAC, CXCL13, IP-10, MCP-3, CCL1, CXCL1, INF-g, CCL26, and MIF (hereinafter Protein Group F); or xxviii) determining the protein level 3U expression of at least one protein selected from the group consisting of MIP-1a, MMP-9, MDC, CCL24, CX3CL1, MIP-3a, MCP-1, TNF-a, IL-8, MCP-4, RANTES, IL-1b, MCP-2, IL-10, I-TAC, CXCL13, IP-10, MCP-3, CCL1, CXCL1, INF-g, CCL26, MIF, IL-16, IL-6, TECK, IL-2, MIP-3b, CXCL2, MIG, and CXCL5 (hereinafter Protein Group G),
   thereby characterizing the glatiramer acetate related drug substance or drug product of step a).

The present invention also provides a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) i) determining the protein level expression of at least one protein produced by a gene selected from the group consisting of Gene Group 1; ii) determining the protein level expression of at least one protein produced by a gene selected from the group consisting of Gene Group 2, wherein if IL1B, IL10, or MMP9 is the at least one gene selected in part (c)(ii), then selecting at least a second different gene from the group other than IL1B, IL10, or MMP9; iii) determining the protein level expression of at least one protein produced by a gene selected from the group consisting of Gene Group 3; iv) determining the protein level expression of at least one protein produced by a gene selected from the group consisting of Gene Group 4; v) determining the protein level expression of at least one protein produced by a gene selected from the group consisting of Gene Group 5; vi) determining the protein level expression of at least one protein produced by a gene selected from the group consisting of Gene Group 6; vii) determining the protein level expression of at least one protein produced by a gene selected from the group consisting of Gene Group 7; viii) determining the protein level expression of at least one protein produced by a gene selected from the group consisting of Gene Group 8; ix) determining the protein level expression of at least one protein produced by a gene selected from the group consisting of Gene Group 9; x) determining the protein level expression of at least one protein produced by a gene selected from the group consisting of Gene Group 10; xi) determining the protein level expression of at least one protein produced by a gene selected from the group consisting of Gene Group 11; xii) determining the protein level expression of at least one protein produced by a gene selected from the group consisting of Gene Group 12; xiii) determining the protein level expression of at least one protein produced by a gene selected from the group consisting of Gene Group 13; xiv) determining the protein level expression of at least one protein produced by a gene selected from the group consisting of Gene Group 14; xv) determining the protein level expression of at least one protein produced by a gene selected from the group consisting of Gene Group 15; xvi) determining the protein level expression of at least one protein produced by a gene selected from the group consisting of Gene Group 16; xvii) determining the protein level expression of at least one protein produced by a gene selected from the group consisting of Gene Group 17; xviii) determining the protein level expression of at least one protein produced by a gene selected from the group consisting of Gene Group 18; xix) determining the protein level expression of at least one protein produced by a gene selected from the group consisting of Gene Group 19; xx) determining the protein level expression of at least one protein produced by a gene selected from the group consisting of Gene Group 20; xxi) determining the protein level expression of at least one protein produced by a gene selected from the group consisting of Gene Group 21; xxii) determining the protein level expression of at least one protein produced by a gene selected from the group consisting of Gene Group 22; xxiii) determining the protein level expression of at least one protein produced by a gene selected from the group consisting of Gene Group 23; xxiv) determining the protein level expression of at least one protein produced by a gene selected from the group consisting of Gene Group 24; xxv) determining the protein level expression of at least one protein produced by a gene selected from the group consisting of Gene Group 25; xxvi) determining the protein level expression of at least one protein produced by a gene selected from the group consisting of Gene Group 26; xxix) determining the protein level expression of at least one protein produced by a gene selected from the group consisting of Gene Group 29; or xxx) determining the protein level expression of at least one protein produced by a gene selected from the group consisting of Gene Group 30,
   thereby characterizing the glatiramer acetate related drug substance or drug product of step a).

### BRIEF DESCRIPTION OF THE DRAWINGS

FIG. 1: Framework of studies conducted in mouse splenocytes and human THP-1 monocyte cell line.

FIG. 2: Roadmap for human THP-1 monocyte cell line study.

FIG. 3: Levels of Copaxone® measured over time in cell culture medium from human THP-1 monocyte cell line. Copaxone® concentration in medium over time remains steady in the range of 44-52 μg/mL over 24 hours.

FIGS. 4A-D: Differentially expressed genes in human THP-1 monocyte cell line (a) Increased expression of IL10 with GA treatment at 6 hours for the single IL10 probeset on the array (207433_at) (FDR adjusted p<3.1e-9); (b-d) Increased expression of IL1RN with GA treatment at 6 hours for multiple probesets (adjusted p values as shown).

FIG. 5: Pathways enriched in experimental systems in mouse splenocytes, human THP-1 monocyte cell line and PBMCs from MS patients among top genes modulated by Copaxone® at 6 hours (subject to FC and adjusted p value filters of 1.5 and 1e-5, respectively). The volcano plot show-log (adjusted p value) for the enrichment plotted versus the fold enrichment score from DAVID for each pathway. A selected subset of pathways are labeled.

FIG. 6: Probeset for cytokine-cytokine receptor interaction pathway genes significantly modulated by Copaxone® at 6 hours in human THP-1 monocyte cell line (subject to FC and adjusted p value filters of 1.5 and 1e-5, respectively). The volcano plot show-log (adjusted p value) for differential pexpression plotted versus the fold change from LIMMA for each probeset.

FIG. 7: MMP9 is significantly upregulated by Probioglat stimulation in human THP-1 monocyte cell line compared to Copaxone® stimulation at 6 and 24 hours (FDR adjusted p values for each timepoint are 2.74e-6, 0.098, and 0.004 for 6, 12, and 24 hours, respectively).

FIG. 8: CD14 expression in human THP-1 monocyte cell line is significantly higher with Probioglat stimulation compared to Copaxone® stimulation at 6 hours.

FIG. 9: Focus on the “response to LPS” pathway as differentially expressed by Probioglat versus Copaxone® at 6 hours in human THP-1 monocyte cell line.

FIG. 10: Pathway-level analysis depicts enrichment among genes upregulated by Probioglat stimulation compared with Copaxone® at 6 hours in human THP-1 monocyte cell line (a) Pathways enriched among genes upregulated by Probioglat stimulation compared with GA at 6 hours. The volcano plot shows −log (adjusted p value) for the enrichment plotted versus the fold enrichment score from DAVID for each pathway; (b) Focus on response to LPS pathway, differentially expressed by Probioglat versus GA at 6 hours. The volcano plot shows −log (adjusted p value) for differential expression plotted versus the fold change from LIMMA for each probeset. A selected subset of pathways are labeled.

FIG. 11: Each present probeset for ICAM1 is significantly upregulated by Probioglat stimulation compared to Copaxone® stimulation at 6 hours in human THP-1 monocyte cell line.

FIG. 12: CISH is downregulated by Probioglat stimulation compared to Copaxone® stimulation at 6 hours in human human THP-1 monocyte cell line.

FIG. 13: Upregulation of IL10 with GA treatment in all three studies in mouse splenocytes, human THP-1 monocyte cell line and PBMCs from MS patients.

FIG. 14: Overlap of genes significantly modulated by GA between studies in three model systems in mouse splenocytes, human THP-1 monocyte cell line and PBMCs from MS patients.

FIGS. 15A-B: Higher levels of (a) CD14 and (b) CD40 (bottom) are induced by a Probioglat generic than by Copaxone® or other generics in human monocytes.

FIG. 16: Expression of IL1B in Copaxone® and multiple generics in mouse splenocytes.

FIG. 17: Expression of CD44 in Copaxone and Escadra generic in human monocytes.

FIG. 18: IL27 expression in Copaxone® and various generics in mouse splenocytes.

FIG. 19: MMP9 expression induced by Copaxone®, Probioglat, and other generics in human monocytes.

FIG. 20: Significant gene expression difference are observed between Copaxone® and the purported generics Natco and Escadra in many genes, including several relevant to MS.

FIG. 21: MMP14 expression is significantly elevated by TV-5010 relative to Copaxone® in mouse splenocytes.

FIG. 22: The expression of PGRMC1 and IL1B shows significantly different expression following stimulation with proposed generics from different manufacturers in human THP-1 monocytes.

FIG. 23: Pathway-level analysis depicts enrichment among genes upregulated by Copaxone® stimulation compared with mannitol control at 6 hours in human THP-1 monocyte cell line.

FIG. 24: IL1RN is significantly upregulated by Copaxone stimulation in human THP-1 monocyte cell line compared to mannitol control at 6 hours (FDR adjusted p value<2.8e-10).

FIG. 25a: Higher level of CYP1B1 is induced by Polimunol generic than by Copaxone® in human THP-1 monocyte cell line at 6 hours (FDR adjusted p values<1.5e-10).

FIG. 25b: Higher level of GPR68 is induced by Polimunol generic than by Copaxone® in human THP-1 monocyte cell line at 6 hours (FDR adjusted p values<1.5e-5).

FIG. 26: Lower level of ADRB2 is induced by Polimunol generic than by Copaxone® in human THP-1 monocyte cell line at 6 hours (FDR adjusted p value<0.009).

FIGS. 27A-E: Copaxone® treatment in mouse splenocytes upregulated (a-b) anti-inflammatory cytokines and (c-d) markers of regulatory T cells, and (e) downregulated pro-inflammatory cytokines.

FIG. 28: Pathways enriched among probesets modulated by Copaxone® relative to medium in splenocytes from mice immunized with Copaxone®.

FIG. 29: IL18 expression is reduced to a greater extent by Copaxone® than Polimunol, regardless of which substance is utilized for the immunization in mouse splenocytes.

FIG. 30: Pathways enriched among top probesets upregulated by Polimunol relative to Copaxone® (in Copaxone®-immunized mice).

FIG. 31: Pathways enriched among top probesets upregulated by Polimunol relative to Copaxone® (in Polimunol-immunized mice).

FIG. 32a: IL-18 signaling pathway (from Dinarello, Front Immunol, 2013; FIG. 1).

FIG. 32b: Mice definicient for IL-18 are resistant to EAE (from Shi, J. Immuno, 2000; FIG. 1a).

FIG. 33: Type I interferon signaling pathway (from Trinchieri et al, Journal of Experimental Medicine, 2010; FIG. 1)

FIGS. 34A-C: Anti-inflammatory gene IL1RN is significantly upregulated by Copaxone in human THP-1 monocyte cell line.

FIG. 35: Pathways enriched among top probesets modulated by Copaxone relative to mannitol control in human THP-1 monocyte cell line.

FIG. 36: Genes in the cytokine-cytokine receptor interaction pathway modulated by Copaxone® relative to mannitol control (each point represents a probeset, labeled with its gene annotation) in human THP-1 monocyte cell line.

FIG. 37: CYP1B1 is significantly upregulated by Polimunol relative to Copaxone®.

FIG. 38: Pathways significantly enriched among top probesets differentially expressed between Polimunol and Copaxone®.

FIG. 39: Genes in the cytokine-cytokine receptor interaction pathway, which is significantly enriched among top probesets differentially expressed between Polimunol and Copaxone® (each point represents a probeset, labeled with its gene annotation).

FIG. 40a: Volcano plot showing probesets driving the enrichment of the response to lipopolysaccharide pathway among probesets upregulated by Polimunol relative to Copaxone®. The dashed line indicates significance level of adjusted p value<0.05.

FIG. 40b: Volcano plot showing probesets driving the enrichment of the response to lipopolysaccharide pathway among probesets upregulated by Probioglat relative to Copaxone® in prior study (FDA-2014-P-0933-0001 available at www.regulations.gov/#?documentDetail;D=FDA-2014-P-0933-0001, and also described herein). The dashed line indicates significance level of adjusted p value<0.05.

FIG. 41: Expression levels of genes differing between Probioglat and GA (a) MMP9 is significantly upregulated following stimulation by Probioglat compared to GA at 6 and 24 hours (FDR-adjusted p values for the single MMP9 probeset on the chip, 203936_s_at, are 2.74e-6, 0.098, and 0.004 for the 6, 12, and 24 hour timepoints, respectively); (b) CD14 expression is significantly higher with stimulation by Probioglat compared to GA at 6 hours (the single CD14 probeset on the chip is shown, 201743_at); (c) Both present ICAM1 probesets are significantly upregulated following stimulation by Probioglat compared to GA at 6 hours (A: probeset 202637_s_at; B: probeset 202638_s_at); (d) CISH is downregulated following stimulation by Probioglat compared to GA at 6 hours (both present probesets are shown, A: probeset 223961_s_at; B: probeset 223377_x_at).

FIG. 42: Expression levels of genes differing between Probioglat and GA by qRT-PCR in primary human monocytes. CCL2 (p<0.009), CCL5 (p<0.029), CXCL10 (p<0.020), MMP9 (p<0.009), and IL1RN (p<0.013) are expressed more highly under Probioglat stimulation relative to GA stimulation.

FIG. 43: Principal Component Analysis (PCA; a multivariate approach) showed that the main effect in the first principal component remained due to treatment effects after batch correction (a) First principal component accounts for experimental factor (treatment time 6, 12, 24 hours); (b) PCA demonstrates batch effect correction required for scan date (legand as in (c)); (c) After batch correction, PCA shows more uniform distribution of scan dates.

FIG. 44: Box plots showing maximum and minimum expression values for Probioglat (red lines) and Copaxone (blue lines).

FIGS. 45A-B: Protein levels at 24 hours are consistent with upregulation of pro-inflammatory mRNA markers by Probioglat vesus Copaxone® treatment at 6h (a) CCL, CXCL10 and MMP-9 protein levels are higher with Probioglat versus Copaxone at 24 hr, consistent with mRNA level observations by RT-PCR and microarray in THP-1 cells; (b) CCL2 and IL-10 protein levels are higher with Probioglat versus Copaxone at 24 hr, consistent with mRNA level observations by microarray (not tested by RT-PCR in THP-1 cells.

FIG. 46: Number of relapses reported to the Patient Support Program in Mexico.

FIGS. 47A_E: Levels of Secreted Protein with Polimunol Versus GA Treatment: Levels of IFNg, TNFa, MIP1a (CCL3), IL-8 (CXCL8), and IL-10 were higher with Polimunol versus GA treatment.

FIGS. 48A-E: Levels of Secreted Protein with Polimunol Versus GA Treatment: Consistent with gene level data, levels of MMP-9, MCP-1 (CCL2), RANTES (CCL5), Gro-a (CXCL1), and IL-1b were higher with Polimunol versus GA treatment.

FIGS. 49A-E: Levels of Secreted Protein with Probioglat Versus GA Treatment: Levels of IFNg, TNFa, MIP1a (CCL3), IL-8 (CXCL8), and IL-10 were higher with Probioglat versus GA treatment.

FIGS. 50A-D: Levels of Secreted Protein with Probioglat Versus GA Treatment: Consistent with gene level data, levels of MMP-9, MCP-1 (CCL2), RANTES (CCL5), and IP-10 (CXCL10) were higher with Probioglat versus GA treatment.

### DETAILED DESCRIPTION OF THE INVENTION

### Embodiments of the Invention

The present invention provides a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) i) determining the level of expression of at least one gene selected from the group consisting of Gene Group 1; ii) determining the level of expression of at least one gene selected from the group consisting of Gene Group 2, wherein if IL1B, IL10, or MMP9 is the at least one gene selected in part (c)(ii), then selecting at least a second different gene from the group other than IL1B, IL10, or MMP9; iii) determining the level of expression of at least one gene selected from the group consisting of Gene Group 3; iv) determining the level of expression of at least one gene selected from the group consisting of Gene Group 4; v) determining the level of expression of at least one gene selected from the group consisting of Gene Group 5; vi) determining the level of expression of at least one gene selected from the group consisting of Gene Group 6; vii) determining the level of expression of at least one gene selected from the group consisting of Gene Group 7; viii) determining the level of expression of at least one gene selected from the group consisting of Gene Group 8; ix) determining the level of expression of at least one gene selected from the group consisting of Gene Group 9; x) determining the level of expression of at least one gene selected from the group consisting of Gene Group 10; xi) determining the level of expression of at least one gene selected from the group consisting of Gene Group 11; xii) determining the level of expression of at least one gene selected from the group consisting of Gene Group 12; xiii) determining the level of expression of at least one gene selected from the group consisting of Gene Group 13; xiv) determining the level of expression of at least one gene selected from the group consisting of Gene Group 14; xv) determining the level of expression of at least one gene selected from the group consisting of Gene Group 15; xvi) determining the level of expression of at least one gene selected from the group consisting of Gene Group 16; xvii) determining the level of expression of at least one gene selected from the group consisting of Gene Group 17; xviii) determining the level of expression of at least one gene selected from the group consisting of Gene Group 18; xix) determining the level of expression of at least one gene selected from the group consisting of Gene Group 19; xx) determining the level of expression of at least one gene selected from the group consisting of Gene Group 20; xxi) determining the level of expression of at least one gene selected from the group consisting of Gene Group 21; xxii) determining the level of expression of at least one gene selected from the group consisting of Gene Group 22; xxiii) determining the level of expression of at least one gene selected from the group consisting of Gene Group 23; xxiv) determining the level of expression of at least one gene selected from the group consisting of Gene Group 24; xxv) determining the level of expression of at least one gene selected from the group consisting of Gene Group 25; xxvi) determining the level of expression of at least one gene selected from the group consisting of Gene Group 26; xxix) determining the level of expression of at least one gene selected from the group consisting of Gene Group 29; or xxx) determining the level of expression of at least one gene selected from the group consisting of Gene Group 30,
   thereby characterizing the glatiramer acetate related drug substance or drug product of step a).

In some embodiments of the present invention step (c) comprises i) determining the level of expression of at least one gene selected from the group consisting of Gene Group 1; ii) determining the level of expression of at least one gene selected from the group consisting of Gene Group 3; or vi) determining the level of expression of at least one gene selected from the group consisting of Gene Group 6.

In some embodiments of the present invention, all genes Gene Group 1, are selected for determining the level of expression.

In some embodiments of the present invention, all genes ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, LPHN1, OLAH, PATZ1, PDK1, POLI, REEP5, RPS6KA2, SEC31A, SETBP1, SNRPA1, SYNCRIP, TNFSF9, TOMM40, TPM1, TSPAN13, UBAP2, VAV3, VDAC2, and ZFAND6, are selected for determining the level of expression.

In some embodiments of the present invention, if the at least one gene selected in part(c)(i) is IL10, the process comprises the step of selecting at least a second different gene from the group of (c)(i) and (c)(ii) other than IL10.

In some embodiments of the present invention, if the at least one gene selected in part(c) (ii) is CARD15, CCL2, CCL5, CD14, IL10, THBD, or NFKBIA, the process comprises the step of selecting at least a second different gene from the group of (c)(i) and (c)(ii) other than CARD15, CCL2, CCL5, CD14, IL10, THBD, or NFKBIA.

In one or more embodiments of the present invention, if two or more genes are selected in step (c), then the second or additional gene selected is different from the other selected gene or genes

In one or more embodiments of the present invention, the level of expression is determined for all genes identified in Table 5 or Table 12 to be involved in one or more than one pathway.

In one or more embodiments of the present invention, the level of expression is determined for at least one gene identified in Table 5 or Table 12 to be involved in at least one pathway.

In one or more embodiments of the present invention, the level of expression is determined for at least one gene identified in Table 5 or Table 12 to be involved in two or more pathways.

In one or more embodiments of the present invention, the level of expression is determined for at least one gene identified in Table 5 or Table 12 to be involved in three or more pathways.

In one or more embodiments of the present invention, the level of expression is determined for at least one gene identified in Table 5 or Table 12 to be involved in four or more pathways.

In one or more embodiments of the present invention, the level of expression is determined for at least one gene identified in Table 5 or Table 12 to be involved in five or more pathways.

In one or more embodiments of the present invention, the level of expression is determined for at least one gene identified in Table 5 or Table 12 to be involved in six pathways.

In one or more embodiments of the present invention, the level of expression is determined for at least one gene which is involved in only one pathway set forth in Table 5 or Table 12.

In one or more embodiments of the present invention, the level of expression is determined for at least two genes identified in Table 5 or Table 12 to be involved in the same pathway.

In one or more embodiments of the present invention, the level of expression is determined for at least three genes identified in Table 5 or Table 12 to be involved in the same pathway.

In one or more embodiments of the present invention, the level of expression is determined for at least four genes identified in Table 5 or Table 12 to be involved in the same pathway.

In one or more embodiments of the present invention, the level of expression is determined for at least five genes identified in Table 5 or Table 12 to be involved in the same pathway.

In one or more embodiments of the present invention, the level of expression is determined for at least six genes identified in Table 5 or Table 12 to be involved in the same pathway.

In one or more embodiments of the present invention, the level of expression is determined for all genes identified in Table 6 to be involved in one or more than one pathway.

In one or more embodiments of the present invention, the level of expression is determined for at least one gene identified in Table 6 to be involved in at least one pathway.

In one or more embodiments of the present invention, the level of expression is determined for at least one gene identified in Table 6 to be involved in two or more pathways.

In one or more embodiments of the present invention, the level of expression is determined for at least one gene identified in Table 6 to be involved in three or more pathways.

In one or more embodiments of the present invention, the level of expression is determined for at least one gene identified in Table 6 to be involved in four or more pathways.

In one or more embodiments of the present invention, the level of expression is determined for at least one gene identified in Table 6 to be involved in five or more pathways.

In one or more embodiments of the present invention, the level of expression is determined for at least one gene identified in Table 6 to be involved in six or more pathways.

In one or more embodiments of the present invention, the level of expression is determined for at least one gene which is involved in only one pathway set forth in Table 6.

In one or more embodiments of the present invention, the level of expression is determined for at least two genes identified in Table 6 to be involved in the same pathway.

In one or more embodiments of the present invention, the level of expression is determined for at least three genes identified in Table 6 to be involved in the same pathway.

In one or more embodiments of the present invention, the level of expression is determined for at least four genes identified in Table 6 to be involved in the same pathway.

In one or more embodiments of the present invention, the level of expression is determined for at least five genes identified in Table 6 to be involved in the same pathway.

In one or more embodiments of the present invention, the level of expression is determined for at least six genes identified in Table 6 to be involved in the same pathway.

In one or more embodiments of the present invention, contacting the mammalian cells in step (b) comprises i) administering to a mammal a predetermined amount of glatiramer acetate related drug substance or drug product of step (a), or ii) incubating the cells with an amount of the glatiramer acetate related drug substance or drug product of step (a), or a combination thereof; and wherein step (c) comprises i) determining the level of expression of at least one gene selected from the group consisting of Gene Group 1, thereby characterizing the glatiramer acetate related drug substance or drug product of step (a).

In one or more embodiments of the present invention, contacting the mammalian cells in step (b) comprises i) administering to a mammal a predetermined amount of glatiramer acetate related drug substance or drug product of step (a), and ii) obtaining cells from the mammal at one or more predetermined time points; and wherein step (c) comprises determining the level of expression of at least one gene selected from the group consisting of Gene Group 1, thereby characterizing the glatiramer acetate related drug substance or drug product of step (a).

In one or more embodiments, the mammal is human and the cells are peripheral mononuclear blood cells.

In one or more embodiments, the predetermined time point is 0, 1, 2, or 3 months.

In one or more embodiments, contacting the mammalian cells in step (b) comprises incubating monocytic cell line cells with an amount of the glatiramer acetate related drug substance or drug product of step (a); and wherein step (c) comprises determining the level of expression of at least one gene selected from the group consisting of Gene Group 1; ii) determining the level of expression of at least one gene selected from the group consisting of Gene Group 2; iii) determining the level of expression of at least one gene selected from the group consisting of Gene Group 3; iv) determining the level of expression of at least one gene selected from the group consisting of Gene Group 4; v) determining the level of expression of at least one gene selected from the group consisting of Gene Group 5; vi) determining the level of expression of at least one gene selected from the group consisting of Gene Group 6; vii) determining the level of expression of at least one gene selected from the group consisting of Gene Group 7; viii) determining the level of expression of at least one gene selected from the group consisting of Gene Group 8; ix) determining the level of expression of at least one gene selected from the group consisting of Gene Group 9; x) determining the level of expression of at least one gene selected from the group consisting of Gene Group 10; xvii) determining the level of expression of at least one gene selected from the group consisting of Gene Group 17; xviii) determining the level of expression of at least one gene selected from the group consisting of Gene Group 18; xix) determining the level of expression of at least one gene selected from the group consisting of Gene Group 19; xx) determining the level of expression of at least one gene selected from the group consisting of Gene Group 20; xxi) determining the level of expression of at least one gene selected from the group consisting of Gene Group 21; xxii) determining the level of expression of at least one gene selected from the group consisting of Gene Group 22; xxiii) determining the level of expression of at least one gene selected from the group consisting of Gene Group 23; xxiv) determining the level of expression of at least one gene selected from the group consisting of Gene Group 24; xxv) determining the level of expression of at least one gene selected from the group consisting of Gene Group 25; xxvi) determining the level of expression of at least one gene selected from the group consisting of Gene Group 26; xxix) determining the level of expression of at least one gene selected from the group consisting of Gene Group 29; or xxx) determining the level of expression of at least one gene selected from the group consisting of Gene Group 30, thereby characterizing the glatiramer acetate related drug substance or drug product of step (a).

In one or more embodiments, contacting the mammalian cells in step (b) comprises incubating monocytic cell line cells with an amount of the glatiramer acetate related drug substance or drug product of step (a); and wherein step (c) comprises i) determining the level of expression of at least one gene selected from the group consisting of Gene Group 2; ii) determining the level of expression of at least one gene selected from the group consisting of Gene Group 3; iii) determining the level of expression of at least one gene selected from the group consisting of Gene Group 4; iv) determining the level of expression of at least one gene selected from the group consisting of Gene Group 5; v) determining the level of expression of at least one gene selected from the group consisting of Gene Group 6; vi) determining the level of expression of at least one gene selected from the group consisting of Gene Group 7; or, vii) determining the level of expression of at least one gene selected from the group consisting of Gene Group 8; ix) determining the level of expression of at least one gene selected from the group consisting of Gene Group 9; x) determining the level of expression of at least one gene selected from the group consisting of Gene Group 10; xvii) determining the level of expression of at least one gene selected from the group consisting of Gene Group 17; xviii) determining the level of expression of at least one gene selected from the group consisting of Gene Group 18; xix) determining the level of expression of at least one gene selected from the group consisting of Gene Group 19; xx) determining the level of expression of at least one gene selected from the group consisting of Gene Group 20; xxi) determining the level of expression of at least one gene selected from the group consisting of Gene Group 21; xxii) determining the level of expression of at least one gene selected from the group consisting of Gene Group 22; xxiii) determining the level of expression of at least one gene selected from the group consisting of Gene Group 23; xxiv) determining the level of expression of at least one gene selected from the group consisting of Gene Group 24; xxv) determining the level of expression of at least one gene selected from the group consisting of Gene Group 25; xxvi) determining the level of expression of at least one gene selected from the group consisting of Gene Group 26; xxix) determining the level of expression of at least one gene selected from the group consisting of Gene Group 29; or xxx) determining the level of expression of at least one gene selected from the group consisting of Gene Group 30, thereby characterizing the glatiramer acetate related drug substance or drug product of step a).

In one or more embodiments of the present invention, the mammalian cells are THP-1 cells.

In one or more embodiments of the present invention, contacting the mammalian cells in step (b) comprises i) immunizing a mammal with a predetermined amount of glatiramer acetate related drug substance or drug product, ii) preparing a culture of cells from the mammal of step i) at one or more predetermined time points after immunization, and iii) incubating cells from the culture of cells obtained from the mammal with an amount of the glatiramer acetate related drug substance or drug product of step (a); and wherein step (c) comprises i) determining the level of expression of at least one gene selected from the group consisting of Gene Group 1; xi) determining the level of expression of at least one gene selected from the group consisting of Gene Group 11; xii) determining the level of expression of at least one gene selected from the group consisting of Gene Group 12; xiii) determining the level of expression of at least one gene selected from the group consisting of Gene Group 13; xiv) determining the level of expression of at least one gene selected from the group consisting of Gene Group 14; xv) determining the level of expression of at least one gene selected from the group consisting of Gene Group 15; or xvi) determining the level of expression of at least one gene selected from the group consisting of Gene Group 16, thereby characterizing the glatiramer acetate related drug substance or drug product of step (a).

In one or more embodiments of the present invention, the glatiramer acetate related drug substance or drug product of step (iii) is the same glatiramer acetate related drug substance or drug product of step (i).

In one or more embodiments of the present invention, the glatiramer acetate related drug substance or drug product of step (iii) is a different glatiramer acetate related drug substance or drug product of step (i).

In one or more embodiments of the present invention, the incubation is for about 24 hours, for about 12 hours, or for about 6 hours.

In one or more embodiments of the present invention, the predetermined time point after immunization is 3 days.

In one or more embodiments of the present invention, the contacting of step (b) is in a cell culture.

In one or more embodiments of the present invention, the culture is a primary culture.

In one or more embodiments of the present invention, the contacting of step (b) is in a mammal.

In one or more embodiments of the present invention, the mammal is a rodent or human.

In one or more embodiments of the present invention, the glatiramer acetate related drug substance or drug product is other than glatiramer acetate drug substance or drug product.

In one or more embodiments of the present invention, the cell is of a type i) selected from the group of cell types consisting of FoxP3+ T cells, regulatory T cells, natural killer T cells, T helper 2 cells, CD8+ T cells, CD4+ T cells, B cells, macrophage cells, monocyte cells, eosinophils, dendritic cells, granulocytes, megakaryocytes, and myeloid progenitors; ii) selected from the group of cell types identified in Table 9; iii) selected from the group of cell types identified in Table 10; or iv) selected from the group of cell types identified in Table 11.

In one or more embodiments of the present invention, the process of characterizing two or more glatiramer acetate related drug substances or drug products further comprises obtaining characteristics of each of the glatiramer acetate related drug substances or drug products; and comparing the characteristics of the drug related substances or drug products, thereby discriminating between glatiramer acetate related drug substances or drug products.

In one or more embodiments of the present invention, the mammal is a rodent or human.

In one or more embodiments of the present invention, the level of expression is determined in hematological cells.

In one or more embodiments of the present invention, the level of expression is determined in splenocytes.

In one or more embodiments of the present invention, the level of expression is determined in monocytes.

In one or more embodiments of the present invention, the monocytes are THP-1.

In one or more embodiments of the present invention, the level of expression is determined in peripheral blood mononuclear cells.

In one or more embodiments of the present invention, the peripheral blood mononuclear cells are from a human.

In one or more embodiments of the present invention, the human has previously been treated with a glatiramer acetate related drug substance or drug product.

In one or more embodiments of the present invention, the human is a naïve human.

In one or more embodiments of the present invention, the human is a glatiramoid naïve human.

In one or more embodiments of the present invention, the human is afflicted with RRMS.

In one or more embodiments of the present invention, the rodent is a mouse.

In one or more embodiments of the present invention, the mouse is a female (SJL X BALB/C) F1 mouse.

In one or more embodiments of the present invention, the mouse is about 8 to 12 weeks old.

In one or more embodiments of the present invention, the primary culture is a culture of spleen cells.

In some embodiments of the present invention, the primary culture is a culture of lymph node cells.

In some embodiments of the present invention, the primary culture of spleen cells is prepared about 3 days after immunization.

In one or more embodiments of the present invention, the glatiramer acetate related drug substance is a glatiramoid or the glatiramer acetate related drug product comprises a glatiramoid.

In one or more embodiments of the present invention, the glatiramer acetate related drug substance is a glatiramoid other than glatiramer acetate related drug substance, or the glatiramer acetate related drug product comprises a glatiramoid other than glatiramer acetate drug substance.

In one or more embodiments of the present invention, process for characterizing a glatiramer acetate related drug substance or drug product comprising the steps of:
a) obtaining a batch of the glatiramer acetate related drug substance or drug product;
b) contacting a first group of mammalian cells with an amount of the glatiramer acetate related drug substance or drug product of step a);
c) contacting a second group of mammalian cells of the same type with an amount of a reference standard; and
d) determining a set of genes differentially expressed by the first group after the contacting of step (b) relative to genes expressed by the second group after the contacting of step (c),
   thereby characterizing the glatiramer acetate related drug substance or drug product of step a).

In one or more embodiments of the present invention, the cells are THP-1 cells.

In one or more embodiments of the present invention, the reference standard is glatiramer acetate related drug substance or drug product.

In one or more embodiments of the present invention, the reference standard is mannitol

In one or more embodiments of the present invention, the reference standard is medium.

In one or more embodiments of the present invention, the determining step (d) comprises comparing the expression of genes expressed by the first group to the expression of genes expressed by the second group.

In one or more embodiments of the present invention, the determining step (d) comprises comparing the expression of genes by bother the first group of cells and by the second group of cells to expression of the genes by the same type of cells exposed to mannitol or medium.

In one or more embodiments of the present invention, process for discriminating between glatiramer acetate related drug substances or drug products comprising the step of characterizing two or more glatiramer acetate related drug substances or drug products to obtain characteristics of each of the glatiramer acetate related drug substances or drug products; and comparing the characteristics of the glatiramer acetate related drug substances or drug products, thereby discriminating between glatiramer acetate related drug substances or drug products.

In some embodiments of the present invention in a process for producing a drug product comprising a glatiramer acetate related drug substance which involves an array of testing, the improvement comprising including in the array of testing the steps of:
a) characterizing the glatiramer acetate related drug substance according to the process of any one of the above characterization methods; and
b) i) including the batch of the glatiramer acetate related drug substance in the production of the drug product if the level of expression of one or more genes selected from the group consisting of Gene Group 1 is not substantially identical to the level of expression by the same type of cells in the absence of the glatiramer acetate related drug substance under the same conditions; ii) including the batch of the glatiramer acetate related drug substance in the production of the drug product if the level of expression of one or more genes selected from the group consisting of Gene Group 2 is substantially identical to the level of expression by the same type of cells in the presence of the glatiramer acetate drug substance under the same conditions; iii) including the batch of the glatiramer acetate related drug substance in the production of the drug product if the level of expression of one or more genes selected from the group consisting of Gene Group 3 is substantially identical to the level of expression by the same type of cells in the presence of the glatiramer acetate drug substance under the same conditions; iv) including the batch of the glatiramer acetate related drug substance in the production of the drug product if the level of expression of one or more genes selected from the group consisting of Gene Group 4 is substantially identical to the level of expression by the same type of cells in the presence of the glatiramer acetate drug substance under the same conditions; v) including the batch of the glatiramer acetate related drug substance in the production of the drug product if the level of expression of one or more genes selected from the group consisting of Gene Group 5 is substantially identical to the level of expression by the same type of cells in the presence of the glatiramer acetate drug substance under the same conditions; vi) including the batch of the glatiramer acetate related drug substance in the production of the drug product if the level of expression of one or more genes selected from the group consisting of Gene Group 6, is substantially identical to the level of expression by the same type of cells in the presence of the glatiramer acetate drug substance under the same conditions; vii) including the batch of the glatiramer acetate related drug substance in the production of the drug product if the level of expression of one or more genes selected from the group consisting of Gene Group 7, is upregulated to the level of expression by the same type of cells in the absence of the glatiramer acetate related drug substance under the same conditions; viii) including the batch of the glatiramer acetate related drug substance in the production of the drug product if the level of expression of one or more genes selected from the group consisting of Gene Group 8, is downregulated to the level of expression by the same type of cells in the absence of the glatiramer acetate related drug substance under the same conditions; or ix) including the batch of the glatiramer acetate related drug substance in the production of the drug product if the level of expression of one or more genes selected from the group consisting of Gene Group 9 is substantially identical to the level of expression by the same type of cells in the presence of the glatiramer acetate drug substance under the same conditions; x) including the batch of the glatiramer acetate related drug substance in the production of the drug product if the level of expression of one or more genes selected from the group consisting of Gene Group 10 is substantially identical to the level of expression by the same type of cells in the presence of the glatiramer acetate drug substance under the same conditions; xi) including the batch of the glatiramer acetate related drug substance in the production of the drug product if the level of expression of one or more genes selected from the group consisting of Gene Group 11 is substantially identical to the level of expression by the same type of cells in the presence of the glatiramer acetate drug substance under the same conditions; xii) including the batch of the glatiramer acetate related drug substance in the production of the drug product if the level of expression of one or more genes selected from the group consisting of Gene Group 12 is substantially identical to the level of expression by the same type of cells in the presence of the glatiramer acetate drug substance under the same conditions; xiii) including the batch of the glatiramer acetate related drug substance in the production of the drug product if the level of expression of one or more genes selected from the group consisting of Gene Group 13 is substantially identical to the level of expression by the same type of cells in the presence of the glatiramer acetate drug substance under the same conditions; xiv) including the batch of the glatiramer acetate related drug substance in the production of the drug product if the level of expression of one or more genes selected from the group consisting of Gene Group 14 is substantially identical to the level of expression by the same type of cells in the presence of the glatiramer acetate drug substance under the same conditions; xv) including the batch of the glatiramer acetate related drug substance in the production of the drug product if the level of expression of one or more genes selected from the group consisting of Gene Group 15 is substantially identical to the level of expression by the same type of cells in the presence of the glatiramer acetate drug substance under the same conditions; xvi) including the batch of the glatiramer acetate related drug substance in the production of the drug product if the level of expression of one or more genes selected from the group consisting of Gene Group 16 is substantially identical to the level of expression by the same type of cells in the presence of the glatiramer acetate drug substance under the same conditions; xvii) including the batch of the glatiramer acetate related drug substance in the production of the drug product if the level of expression of one or more genes selected from the group consisting of Gene Group 17 is substantially identical to the level of expression by the same type of cells in the presence of the glatiramer acetate drug substance under the same conditions; xviii) including the batch of the glatiramer acetate related drug substance in the production of the drug product if the level of expression of one or more genes selected from the group consisting of Gene Group 18 is substantially identical to the level of expression by the same type of cells in the presence of the glatiramer acetate drug substance under the same conditions; xix) including the batch of the glatiramer acetate related drug substance in the production of the drug product if the level of expression of one or more genes selected from the group consisting of Gene Group 19 is substantially identical to the level of expression by the same type of cells in the presence of the glatiramer acetate drug substance under the same conditions; xx) including the batch of the glatiramer acetate related drug substance in the production of the drug product if the level of expression of one or more genes selected from the group consisting of Gene Group 20 is substantially identical to the level of expression by the same type of cells in the presence of the glatiramer acetate drug substance under the same conditions; xxi) including the batch of the glatiramer acetate related drug substance in the production of the drug product if the level of expression of one or more genes selected from the group consisting of Gene Group 21 is substantially identical to the level of expression by the same type of cells in the presence of the glatiramer acetate drug substance under the same conditions; xxii) including the batch of the glatiramer acetate related drug substance in the production of the drug product if the level of expression of one or more genes selected from the group consisting of Gene Group 22 is substantially identical to the level of expression by the same type of cells in the presence of the glatiramer acetate drug substance under the same conditions; xxiii) including the batch of the glatiramer acetate related drug substance in the production of the drug product if the level of expression of one or more genes selected from the group consisting of Gene Group 23 is substantially identical to the level of expression by the same type of cells in the presence of the glatiramer acetate drug substance under the same conditions; xxiv) including the batch of the glatiramer acetate related drug substance in the production of the drug product if the level of expression of one or more genes selected from the group consisting of Gene Group 24 is substantially identical to the level of expression by the same type of cells in the presence of the glatiramer acetate drug substance under the same conditions; xxv) including the batch of the glatiramer acetate related drug substance in the production of the drug product if the level of expression of one or more genes selected from the group consisting of Gene Group 25 is substantially identical to the level of expression by the same type of cells in the presence of the glatiramer acetate drug substance under the same conditions; xxvi) including the batch of the glatiramer acetate related drug substance in the production of the drug product if the level of expression of one or more genes selected from the group consisting of Gene Group 26 is substantially identical to the level of expression by the same type of cells in the presence of the glatiramer acetate drug substance under the same conditions; xxix) including the batch of the glatiramer acetate related drug substance in the production of the drug product if the level of expression of one or more genes selected from the group consisting of Gene Group 29 is substantially identical to the level of expression by the same type of cells in the presence of the glatiramer acetate drug substance under the same conditions; or xxx) including the batch of the glatiramer acetate related drug substance in the production of the drug product if the level of expression of one or more genes selected from the group consisting of Gene Group 30 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 1 is not substantially identical to the level of expression by the same type of cells in the absence of the glatiramer acetate related drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, LPHN1, OLAH, PATZ1, PDK1, POLI, REEP5, RPS6KA2, SEC31A, SETBP1, SNRPA1, SYNCRIP, TNFSF9, TOMM40, TPM1, TSPAN13, UBAP2, VAV3, VDAC2, and ZFAND6 is not substantially identical to the level of expression by the same type of cells in the absence of the glatiramer acetate related drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 2 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 3 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 4 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 5 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 6 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises i) including the batch of the glatiramer acetate related drug substance in the production of the drug product if the level of expression of one or more genes selected from the group consisting of ABI2, ARPC4, CD84, CLU, HFE, and IL10 is upregulated relative to the level of expression by the same type of cells in the absence of the glatiramer acetate related drug substance under the same conditions; or ii) including the batch of the glatiramer acetate related drug substance in the production of the drug product if the level of expression of one or more genes selected from the group consisting of ABCF2, ACP6, AFG3L2, CHAF1A, COX11, LPHN1, NACA, OLAH, POLI, SEC31A, SNRPA1, SYNCRIP, TNFSF9, TOMM40, TSHZ1, TSPAN13, UBAP2, VDAC2, and TSHZ1 is downregulated relative to the level of expression by the same type of cells in the absence of the glatiramer acetate related drug substance under the same conditions.

In one or more embodiments of the present invention, in the process for producing a drug product comprising a glatiramer acetate related drug substance which involves an array of testing, the characterizing the glatiramer acetate related drug substance further comprises, if, one or more genes selected from the group consisting of ABI2, ARPC4, HFE, and IL10 is upregulated relative to the level of expression by the same type of cells in the absence of the glatiramer acetate related drug substance under the same conditions, then including the batch of the glatiramer acetate related drug substance in the production of the drug product.

In one or more embodiments of the present invention, in the process for producing a drug product comprising a glatiramer acetate related drug substance which involves an array of testing, the characterizing the glatiramer acetate related drug substance further comprises, if, one or more genes selected from the group consisting of ACP6, LPHN1, POLI, SEC31A, SYNCRIP, and TSHZ1 is downregulated relative to the level of expression by the same type of cells in the absence of the glatiramer acetate related drug substance under the same conditions, then including the batch of the glatiramer acetate related drug substance in the production of the drug product.

In one or more embodiments of the present invention, in the process for producing a drug product comprising a glatiramer acetate related drug substance which involves an array of testing, the characterizing the glatiramer acetate related drug substance further comprises i) including the batch of the glatiramer acetate related drug substance in the production of the drug product if the level of expression of one or more genes selected from the group consisting of CCL2, CCL5, MMP1, MMP9, CXCL10, CARD15, CD14, ICAM1, BIRC3, THBD, NFKBIA, IL10, PRDM1 is substantially identical to the level of expression by the same type of cells in the presence of glatiramer acetate drug substance under the same conditions; ii) including the batch of the glatiramer acetate related drug substance in the production of the drug product if the level of expression of one or more genes selected from the group consisting of CISH and HSPD1 is substantially identical to the level of expression by the same type of cells in the presence of glatiramer acetate drug substance under the same conditions; iii) including the batch of the glatiramer acetate related drug substance in the production of the drug product if the level of expression of one or more genes selected from the group consisting of CC124, CCR1, CSF1R, CX3CR1, IL27, IFNGR1, IL2RG, and IL7R is not substantially identical to the level of expression by the same type of cells in the absence of the glatiramer acetate drug related substance under the same conditions; iv) including the batch of the glatiramer acetate related drug substance in the production of the drug product if the level of expression of PGRMC1 is upregulated or substantially identical to the level of expression by the same type of cells in the absence of the glatiramer acetate related drug substance under the same conditions; v) including the batch of the glatiramer acetate related drug substance in the production of the drug product if the level of expression of MMP14 is substantially identical to the level of expression by the same type of cells in the presence of glatiramer acetate drug substance under the same conditions; vi) including the batch of the glatiramer acetate related drug substance in the production of the drug product if the level of expression of IL1RN is upregulated to the level of expression by the same type of cells in the absence of the glatiramer acetate related drug substance under the same conditions; or vii) including the batch of the glatiramer acetate related drug substance in the production of the drug product if the level of expression of IL1B is substantially identical to the level of expression by the same type of cells in the presence of glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 5 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 6 is substantially identical to the level of expression by the same type of cells in the presence of the glatiramer acetate drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 7, is upregulated to the level of expression by the same type of cells in the absence of the glatiramer acetate related drug substance under the same conditions.

In one or more embodiments of the present invention, in the process for producing a drug product comprising a glatiramer acetate related drug substance which involves an array of testing, the characterizing the glatiramer acetate related drug substance further comprises including the batch of the glatiramer acetate related drug substance in the production of the drug product if the level of expression of one or more genes selected from the group consisting of Gene Group 8, is downregulated to the level of expression by the same type of cells in the absence of the glatiramer acetate related drug substance under the same conditions.

In some embodiments of the present invention in a process for releasing a drug product comprising a glatiramer acetate related drug substance, which process involves an array of testing, the improvement comprising including in the array of testing the steps of:
a) characterizing the glatiramer acetate related drug product according to the process of any one of the above characterization methods; and
b) i) releasing the batch of the glatiramer acetate related drug product if the level of expression of one or more genes selected from the group consisting of Gene Group 1 is not substantially identical to the level of expression by the same type of cells in the absence of the glatiramer acetate related drug product under the same conditions; ii) releasing the batch of the glatiramer acetate related drug product if the level of expression of one or more genes selected from the group consisting of Gene Group 2 is substantially identical to the level of expression by the same type of cells in the presence of the glatiramer acetate drug product under the same conditions; iii) releasing the batch of the glatiramer acetate related drug product if the level of expression of one or more genes selected from the group consisting of Gene Group 3 is substantially identical to the level of expression by the same type of cells in the presence of the glatiramer acetate drug product under the same conditions; iv) releasing the batch of the glatiramer acetate related drug product if the level of expression of one or more genes selected from the group consisting of Gene Group 4 is substantially identical to the level of expression by the same type of cells in the presence of the glatiramer acetate drug product under the same conditions; v) releasing the batch of the glatiramer acetate related drug product if the level of expression of one or more genes selected from the group consisting of Gene Group 5 is substantially identical to the level of expression by the same type of cells in the presence of the glatiramer acetate drug product under the same conditions; vi) releasing the batch of the glatiramer acetate related drug product if the level of expression of one or more genes selected from the group consisting of Gene Group 6, is substantially identical to the level of expression by the same type of cells in the presence of the glatiramer acetate drug product under the same conditions; vii) releasing the batch of the glatiramer acetate related drug product if the level of expression of one or more genes selected from the group consisting of Gene Group 7, is upregulated to the level of expression by the same type of cells in the absence of the glatiramer acetate related drug substance under the same conditions; viii) releasing the batch of the glatiramer acetate related drug product if the level of expression of one or more genes selected from the group consisting of Gene Group 8, is downregulated to the level of expression by the same type of cells in the absence of the glatiramer acetate related drug substance under the same conditions, ix) releasing the batch of the glatiramer acetate related drug product if the level of expression of one or more genes selected from the group consisting of Gene Group 9 is substantially identical to the level of expression by the same type of cells in the presence of the glatiramer acetate drug product under the same conditions; x) releasing the batch of the glatiramer acetate related drug product if the level of expression of one or more genes selected from the group consisting of Gene Group 10 is substantially identical to the level of expression by the same type of cells in the presence of the glatiramer acetate drug product under the same conditions; xi) releasing the batch of the glatiramer acetate related drug product if the level of expression of one or more genes selected from the group consisting of Gene Group 11 is substantially identical to the level of expression by the same type of cells in the presence of the glatiramer acetate drug substance under the same conditions; xii) releasing the batch of the glatiramer acetate related drug product if the level of expression of one or more genes selected from the group consisting of Gene Group 12 is substantially identical to the level of expression by the same type of cells in the presence of the glatiramer acetate drug substance under the same conditions; xiii) releasing the batch of the glatiramer acetate related drug product if the level of expression of one or more genes selected from the group consisting of Gene Group 13 is substantially identical to the level of expression by the same type of cells in the presence of the glatiramer acetate drug substance under the same conditions; xiv) releasing the batch of the glatiramer acetate related drug product if the level of expression of one or more genes selected from the group consisting of Gene Group 14 is substantially identical to the level of expression by the same type of cells in the presence of the glatiramer acetate drug substance under the same conditions; xv) releasing the batch of the glatiramer acetate related drug product if the level of expression of one or more genes selected from the group consisting of Gene Group 15 is substantially identical to the level of expression by the same type of cells in the presence of the glatiramer acetate drug substance under the same conditions; xvi) releasing the batch of the glatiramer acetate related drug product if the level of expression of one or more genes selected from the group consisting of Gene Group 16 is substantially identical to the level of expression by the same type of cells in the presence of the glatiramer acetate drug substance under the same conditions; xvii) releasing the batch of the glatiramer acetate related drug product if the level of expression of one or more genes selected from the group consisting of Gene Group 17 is substantially identical to the level of expression by the same type of cells in the presence of the glatiramer acetate drug substance under the same conditions; xviii) releasing the batch of the glatiramer acetate related drug product if the level of expression of one or more genes selected from the group consisting of Gene Group 18 is substantially identical to the level of expression by the same type of cells in the presence of the glatiramer acetate drug substance under the same conditions; xix) releasing the batch of the glatiramer acetate related drug product if the level of expression of one or more genes selected from the group consisting of Gene Group 19 is substantially identical to the level of expression by the same type of cells in the presence of the glatiramer acetate drug substance under the same conditions; xx) releasing the batch of the glatiramer acetate related drug product if the level of expression of one or more genes selected from the group consisting of Gene Group 20 is substantially identical to the level of expression by the same type of cells in the presence of the glatiramer acetate drug substance under the same conditions; xxi) releasing the batch of the glatiramer acetate related drug product if the level of expression of one or more genes selected from the group consisting of Gene Group 21 is substantially identical to the level of expression by the same type of cells in the presence of the glatiramer acetate drug substance under the same conditions; xxii) releasing the batch of the glatiramer acetate related drug product if the level of expression of one or more genes selected from the group consisting of Gene Group 22 is substantially identical to the level of expression by the same type of cells in the presence of the glatiramer acetate drug substance under the same conditions; xxiii) releasing the batch of the glatiramer acetate related drug product if the level of expression of one or more genes selected from the group consisting of Gene Group 23 is substantially identical to the level of expression by the same type of cells in the presence of the glatiramer acetate drug substance under the same conditions; xxiv) releasing the batch of the glatiramer acetate related drug product if the level of expression of one or more genes selected from the group consisting of Gene Group 24 is substantially identical to the level of expression by the same type of cells in the presence of the glatiramer acetate drug substance under the same conditions; xxv) releasing the batch of the glatiramer acetate related drug product if the level of expression of one or more genes selected from the group consisting of Gene Group 25 is substantially identical to the level of expression by the same type of cells in the presence of the glatiramer acetate drug substance under the same conditions; xxvi) releasing the batch of the glatiramer acetate related drug product if the level of expression of one or more genes selected from the group consisting of Gene Group 26 is substantially identical to the level of expression by the same type of cells in the presence of the glatiramer acetate drug substance under the same conditions; xxix) releasing the batch of the glatiramer acetate related drug product if the level of expression of one or more genes selected from the group consisting of Gene Group 29 is substantially identical to the level of expression by the same type of cells in the presence of the glatiramer acetate drug substance under the same conditions; or xxx) releasing the batch of the glatiramer acetate related drug product if the level of expression of one or more genes selected from the group consisting of Gene Group 30 is substantially identical to the level of expression by the same type of cells in the presence of the glatiramer acetate drug substance under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 1 is not substantially identical to the level of expression by the same type of cells in the absence of the glatiramer acetate related drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, LPHN1, OLAH, PATZ1, PDK1, POLI, REEP5, RPS6KA2, SEC31A, SETBP1, SNRPA1, SYNCRIP, TNFSF9, TOMM40, TPM1, TSPAN13, UBAP2, VAV3, VDAC2, and ZFAND6 is not substantially identical to the level of expression by the same type of cells in the absence of the glatiramer acetate related drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 2 is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 3 is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 4 is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 5, is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 6 is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug substance further comprises i) releasing the batch of the glatiramer acetate related drug product if the level of expression of one of more genes selected from the group consisting of ABI2, ARPC4, CD84, CLU, HFE, and IL10 is upregulated relative to the level of expression by the same type of cells in the absence of the glatiramer acetate related drug product under the same conditions; or ii) releasing the batch of the glatiramer acetate related drug product if the level of expression of one of more genes selected from the group consisting of ABCF2, ACP6, AFG3L2, CHAF1A, COX11, LPHN1, NACA, OLAH, POLI, SEC31A, SNRPA1, SYNCRIP, TNFSF9, TOMM40, TSHZ1, TSPAN13, UBAP2, VDAC2, and TSHZ1 is downregulated relative to the level of expression by the same type of cells in the absence of the glatiramer acetate related drug product under the same conditions.

In one or more embodiments of the present invention, in the process for releasing a drug product comprising a glatiramer acetate related drug substance which involves an array of testing, the characterizing the glatiramer acetate related drug substance further comprises, if, one or more genes selected from the group consisting of ABI2, ARPC4, HFE, and IL10 is upregulated relative to the level of expression by the same type of cells in the absence of the glatiramer acetate related drug product under the same conditions, then releasing the batch of the glatiramer acetate related drug product.

In one or more embodiments of the present invention, in the process for releasing a drug product comprising a glatiramer acetate related drug substance which involves an array of testing, the characterizing the glatiramer acetate related drug product further comprises, if, one or more genes selected from the group consisting of ACP6, LPHN1, POLI, SEC31A, SYNCRIP, and TSHZ1 is downregulated relative to the level of expression by the same type of cells in the presence of the glatiramer acetate drug product under the same conditions, then releasing the batch of the glatiramer acetate related drug product.

In one or more embodiments of the present invention, in the process for releasing a drug product comprising a glatiramer acetate related drug substance which involves an array of testing, the characterizing the glatiramer acetate related drug product further comprises i) releasing the batch of the glatiramer acetate related drug product if the level of expression of one of more genes selected from the group consisting of CCL2, CCL5, MMP1, MMP9, CXCL10, CARD15, CD14, ICAM1, BIRC3, THBD, NFKBIA, IL10, PRDM1 is substantially identical to the level of expression by the same type of cells in the presence of glatiramer acetate drug substance under the same conditions; ii) releasing the batch of the glatiramer acetate related drug product if the level of expression of one of more genes selected from the group consisting of CISH and HSPD1 is substantially identical to the level of expression by the same type of cells in the presence of glatiramer acetate drug substance under the same conditions; iii) releasing the batch of the glatiramer acetate related drug product if the level of expression of one of more genes selected from the group consisting of CC124, CCR1, CSF1R, CX3CR1, IL27, IFNGR1, IL2RG, and IL7R is not substantially identical to the level of expression by the same type of cells in the absence of the glatiramer acetate drug related substance under the same conditions; iv) releasing the batch of the glatiramer acetate related drug product if the level of expression of PGRMC1 is upregulated or substantially identical to the level of expression by the same type of cells in the absence of the glatiramer acetate related drug substance under the same conditions; v) releasing the batch of the glatiramer acetate related drug product if the level of expression of MMP14 is substantially identical to the level of expression by the same type of cells in the presence of glatiramer acetate drug substance under the same conditions; vi) releasing the batch of the glatiramer acetate related drug product if the level of expression of IL1RN is upregulated to the level of expression by the same type of cells in the absence of the glatiramer acetate related drug substance under the same conditions; or vii) releasing the batch of the glatiramer acetate related drug product if the level of expression of IL1B is substantially identical to the level of expression by the same type of cells in the presence of glatiramer acetate drug substance under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 5, is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 6 is substantially identical to the level of expression by the same type of cells in the presence of the glatiramer acetate drug product under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 7, is upregulated to the level of expression by the same type of cells in the absence of the glatiramer acetate related drug substance under the same conditions.

In one or more embodiments of the present invention, in the process for releasing a drug product comprising a glatiramer acetate related drug substance which involves an array of testing, the characterizing the glatiramer acetate related drug product further comprises releasing the batch of the glatiramer acetate related drug product if the level of expression of one or more genes selected from the group consisting of Gene Group 8, is downregulated to the level of expression by the same type of cells in the absence of the glatiramer acetate related drug substance under the same conditions.

In one or more embodiments of the present invention, a glatiramer acetate related drug product produced by a process of the present invention, wherein the glatiramer acetate related drug product is other than glatiramer acetate drug product.

In one or more embodiments of the present invention, a glatiramer acetate related drug product other than glatiramer acetate drug product which is capable of inducing a level of expression of ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, LPHN1, OLAH, PATZ1, PDK1, POLI, REEP5, RPS6KA2, SEC31A, SETBP1, SNRPA1, SYNCRIP, TNFSF9, TOMM40, TPM1, TSPAN13, UBAP2, VAV3, VDAC2, and ZFAND6 that is not substantially identical to the level of expression of the genes induced in the same tpe of cells and under the same conditions in the absence of the glatiramer acetate related drug product.

In one or more embodiments of the present invention, a glatiramer acetate related drug product, wherein the glatiramer acetate related drug product is capable of inducing a level of expression of Gene Group 1.

In one or more embodiments of the present invention, the glatiramer acetate related drug product, which is capable of upregulating genes ABI2, ARPC4, HFE, and IL10 relative to the level of expression of the genes in the same type of cells and under the same conditions in the absence of the glatiramer acetate related drug product, and capable of downregulating genes ACP6, LPHN1, POLI, SEC31A, SYNCRIP, and TSHZ1 relative to the level of expression of the genes in the same type of cells and under the same conditions in the absence of the glatiramer acetate related drug product.

In one or more embodiments of the present invention, a glatiramer acetate related drug product other than glatiramer acetate drug product which is capable of inducing a level of expression of Gene Group 7 and Gene Group 8, that is not substantially identical to the level of expression of the genes induced in the same type of cells and under the same conditions in the absence of the glatiramer acetate related drug product.

In one or more embodiments of the present invention, the glatiramer acetate related drug product, which is capable of upregulating genes Gene Group 7, relative to the level of expression of the genes in the same type of cells and under the same conditions in the absence of the glatiramer acetate related drug product, and capable of downregulating genes Gene Group 8 relative to the level of expression of the genes in the same type of cells and under the same conditions in the absence of the glatiramer acetate related drug product

In some embodiments of the present invention, the process further comprises i) determining the one or more proteins produced by each of the one of more genes selected in step c); and ii) determining protein level expression for each protein in step i).

In some embodiments of the present invention, the process further comprises i) determining the one or more proteins produced by each of the one of more genes selected in step b); and ii) determining protein level expression for each protein in step i).

In some embodiments of the present invention, the process further comprises determining the set of proteins produced by each gene of the set of genes in step d).

In some embodiments of the present invention, in a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) xxii) determining the protein level expression of at least one protein selected from the group consisting of Protein Group A; xxiii) determining the protein level expression of at least one protein selected from the group consisting of Protein Group B; xxiv) determining the protein level expression of at least one protein selected from the group consisting of Protein Group C; xxv) determining the protein level expression of at least one protein selected from the group consisting of Protein Group D; xxvi) determining the protein level expression of at least one protein selected from the group consisting of Protein Group E; xxvii) determining the protein level expression of at least one protein selected from the group consisting of Protein Group F; or xxviii) determining the protein level expression of at least one protein selected from the group consisting of Protein Group G,
   thereby characterizing the glatiramer acetate related drug substance or drug product of step a).

In some embodiments of the present invention, in a process for characterizing a glatiramer acetate related drug substance or drug product comprising the steps of:
a) obtaining a batch of the glatiramer acetate related drug substance or drug product;
b) contacting mammalian cells with an amount of the glatiramer acetate related drug substance or drug product of step a); and
c) i) determining the protein level expression of at least one protein produced by a gene selected from the group consisting of Gene Group 1; ii) determining the protein level expression of at least one protein produced by a gene selected from the group consisting of Gene Group 2, wherein if IL1B, IL10, or MMP9 is the at least one gene selected in part (c)(ii), then selecting at least a second different gene from the group other than IL1B, IL10, or MMP9; iii) determining the protein level expression of at least one protein produced by a gene selected from the group consisting of Gene Group 3; iv) determining the protein level expression of at least one protein produced by a gene selected from the group consisting of Gene Group 4; v) determining the protein level expression of at least one protein produced by a gene selected from the group consisting of Gene Group 5; vi) determining the protein level expression of at least one protein produced by a gene selected from the group consisting of Gene Group 6; vii) determining the protein level expression of at least one protein produced by a gene selected from the group consisting of Gene Group 7; viii) determining the protein level expression of at least one protein produced by a gene selected from the group consisting of Gene Group 8; ix) determining the protein level expression of at least one protein produced by a gene selected from the group consisting of Gene Group 9; x) determining the protein level expression of at least one protein produced by a gene selected from the group consisting of Gene Group 10; xi) determining the protein level expression of at least one protein produced by a gene selected from the group consisting of Gene Group 11; xii) determining the protein level expression of at least one protein produced by a gene selected from the group consisting of Gene Group 12; x) determining the protein level expression of at least one protein produced by a gene selected from the group consisting of Gene Group 13; xiv) determining the protein level expression of at least one protein produced by a gene selected from the group consisting of Gene Group 14; xv) determining the protein level expression of at least one protein produced by a gene selected from the group consisting of Gene Group 15; xvi) determining the protein level expression of at least one protein produced by a gene selected from the group consisting of Gene Group 16; xvii) determining the protein level expression of at least one protein produced by a gene selected from the group consisting of Gene Group 17; xviii) determining the protein level expression of at least one protein produced by a gene selected from the group consisting of Gene Group 18; xix) determining the protein level expression of at least one protein produced by a gene selected from the group consisting of Gene Group 19; xx) determining the protein level expression of at least one protein produced by a gene selected from the group consisting of Gene Group 20; xxi) determining the protein level expression of at least one protein produced by a gene selected from the group consisting of Gene Group 21; xxii) determining the protein level expression of at least one protein produced by a gene selected from the group consisting of Gene Group 22; xxiii) determining the protein level expression of at least one protein produced by a gene selected from the group consisting of Gene Group 23; xxiv) determining the protein level expression of at least one protein produced by a gene selected from the group consisting of Gene Group 24; xxv) determining the protein level expression of at least one protein produced by a gene selected from the group consisting of Gene Group 25; xxvi) determining the protein level expression of at least one protein produced by a gene selected from the group consisting of Gene Group 26; xxix) determining the protein level expression of at least one protein produced by a gene selected from the group consisting of Gene Group 29; or xxx) determining the protein level expression of at least one protein produced by a gene selected from the group consisting of Gene Group 30,
   thereby characterizing the glatiramer acetate related drug substance or drug product of step a).

In some embodiments of the present invention the process, wherein contacting the mammalian cells in step (b) comprises incubating monocytic cell line cells with an amount of the glatiramer acetate related drug substance or drug product of step (a); and wherein step (c) comprises xxii) determining the protein level expression of at least one protein selected from the group consisting of Protein Group A; xxiii) determining the protein level expression of at least one protein selected from the group consisting of Protein Group B; xxiv) determining the protein level expression of at least one protein selected from the group consisting of Protein Group C; xxv) determining the protein level expression of at least one protein selected from the group consisting of Protein Group D; xxvi) determining the protein level expression of at least one protein selected from the group consisting of Protein Group E; xxvii) determining the protein level expression of at least one protein selected from the group consisting of Protein Group F; or xxviii) determining the protein level expression of at least one protein selected from the group consisting of Protein Group G, thereby characterizing the glatiramer acetate related drug substance or drug product of step (a).

In some embodiments of the present invention, the process wherein the mammalian cells are THP-1 cells.

In some embodiments of the present invention, the process, wherein the incubation is for about 24 hours.

In some embodiments of the present invention, the process, wherein contacting the mammalian cells in step (b) comprises i) immunizing a mammal with a predetermined amount of glatiramer acetate related drug substance or drug product, ii) preparing a culture of cells from the mammal of step i) at one or more predetermined time points after immunization, and iii) incubating cells from the culture of cells obtained from the mammal with an amount of the glatiramer acetate related drug substance or drug product of step (a), thereby characterizing the glatiramer acetate related drug substance or drug product of step (a).

In some embodiments of the present invention, the process, wherein the glatiramer acetate related drug substance or drug product of step (iii) is the same glatiramer acetate related drug substance or drug product of step (i).

In some embodiments of the present invention, the process, wherein the glatiramer acetate related drug substance or drug product of step (iii) is a different glatiramer acetate related drug substance or drug product of step (i).

In some embodiments of the present invention, in a process for producing a drug product comprising a glatiramer acetate related drug substance which involves an array of testing, the improvement comprising including in the array of testing the steps of:
a) characterizing the glatiramer acetate related drug substance according to the process of any one of the above characterization methods; and
b) xxii) including the batch of the glatiramer acetate related drug substance in the production of the drug product if the level of expression of one or more proteins selected from the group consisting of Protein Group A is substantially identical to the level of expression by the same type of cells in the presence of the glatiramer acetate related drug substance under the same conditions; xxiii) including the batch of the glatiramer acetate related drug substance in the production of the drug product if the level of expression of one or more proteins selected from the group consisting of Protein Group B is substantially identical to the level of expression by the same type of cells in the presence of the glatiramer acetate related drug substance under the same conditions; xxiv) including the batch of the glatiramer acetate related drug substance in the production of the drug product if the level of expression of one or more proteins selected from the group consisting of Protein Group C is substantially identical to the level of expression by the same type of cells in the presence of the glatiramer acetate related drug substance under the same conditions; xxv) including the batch of the glatiramer acetate related drug substance in the production of the drug product if the level of expression of one or more proteins selected from the group consisting of Protein Group D is substantially identical to the level of expression by the same type of cells in the presence of the glatiramer acetate related drug substance under the same conditions; xxvi) including the batch of the glatiramer acetate related drug substance in the production of the drug product if the level of expression of one or more proteins selected from the group consisting of Protein Group E is substantially identical to the level of expression by the same type of cells in the presence of the glatiramer acetate related drug substance under the same conditions; xxvii) including the batch of the glatiramer acetate related drug substance in the production of the drug product if the level of expression of one or more proteins selected from the group consisting of Protein Group F is substantially identical to the level of expression by the same type of cells in the presence of the glatiramer acetate related drug substance under the same conditions; or xxviii) including the batch of the glatiramer acetate related drug substance in the production of the drug product if the level of expression of one or more proteins selected from the group consisting of Protein Group G is substantially identical to the level of expression by the same type of cells in the presence of the glatiramer acetate related drug substance under the same conditions.

In some embodiments of the present invention, in a process for releasing a drug product comprising a glatiramer acetate related drug substance, which process involves an array of testing, the improvement comprising including in the array of testing the steps of:
a) characterizing the glatiramer acetate related drug product according to the process of any one of the above characterization methods; and
b) xxii) releasing the batch of the glatiramer acetate related drug product if the level of expression of one or more proteins selected from the group consisting of Protein Group A is substantially identical to the level of expression by the same type of cells in the presence of the glatiramer acetate related drug product under the same conditions; xxiii) releasing the batch of the glatiramer acetate related drug product if the level of expression of one or more proteins selected from the group consisting of Protein Group B is substantially identical to the level of expression by the same type of cells in the presence of the glatiramer acetate related drug product under the same conditions; xxiv) releasing the batch of the glatiramer acetate related drug product if the level of expression of one or more proteins selected from the group consisting of Protein Group C is substantially identical to the level of expression by the same type of cells in the presence of the glatiramer acetate related drug product under the same conditions; xxv) releasing the batch of the glatiramer acetate related drug product if the level of expression of one or more proteins selected from the group consisting of Protein Group D is substantially identical to the level of expression by the same type of cells in the presence of the glatiramer acetate related drug product under the same conditions; xxvi) releasing the batch of the glatiramer acetate related drug product if the level of expression of one or more proteins selected from the group consisting of Protein Group E is substantially identical to the level of expression by the same type of cells in the presence of the glatiramer acetate related drug product under the same conditions; xxvii) releasing the batch of the glatiramer acetate related drug product if the level of expression of one or more proteins selected from the group consisting of Protein Group F is substantially identical to the level of expression by the same type of cells in the presence of the glatiramer acetate related drug product under the same conditions; or xxviii) releasing the batch of the glatiramer acetate related drug product if the level of expression of one or more proteins selected from the group consisting of Protein Group G is substantially identical to the level of expression by the same type of cells in the presence of the glatiramer acetate related drug product under the same conditions.

In some embodiments of the present invention, in a process for discriminating between glatiramer acetate related drug substances or drug products comprising the steps of:
a) characterizing one or more glatiramer acetate related drug substances or drug products according to the process of any one of the above characterization methods to obtain characteristics of each of the glatiramer acetate related drug substances or drug products;
b) obtaining characteristics of a glatiramer acetate drug substance or drug product; and
c) comparing the characteristics of the glatiramer acetate related drug substances or drug products obtained in steps a) and b),
   thereby discriminating between glatiramer acetate related drug substances or drug products.

### ADDITIONAL EMBODIMENTS OF THE INVENTION

In a process for producing a drug product comprising a glatiramer acetate related drug substance, the improvement comprising the steps of:
a) characterizing the glatiramer acetate related drug substance according to the process of any one of the above characterization methods; and
b) i) discarding the batch of the glatiramer acetate related drug substance as unacceptable for inclusion in the drug product if the level of expression of one or more genes selected from the group consisting of Gene Group 1 is substantially identical to the level of expression by the same type of cells in the absence of the glatiramer acetate related drug substance under the same conditions; ii) discarding the batch of the glatiramer acetate related drug substance as unacceptable for inclusion in the drug product if the level of expression of one or more genes selected from the group consisting of Gene Group 2 is not substantially identical to the level of expression by the same type of cells in the presence of the glatiramer acetate drug substance under the same conditions; iii) discarding the batch of the glatiramer acetate related drug substance as unacceptable for inclusion in the drug product if the level of expression of one or more genes selected from the group consisting of Gene Group 3 is not substantially identical to the level of expression by the same type of cells in the presence of the glatiramer acetate drug substance under the same conditions; iv) discarding the batch of the glatiramer acetate related drug substance as unacceptable for inclusion in the drug product if the level of expression of one or more genes selected from the group consisting of Gene Group 4 is not substantially identical to the level of expression by the same type of cells in the presence of the glatiramer acetate drug substance under the same conditions; v) discarding the batch of the glatiramer acetate related drug substance as unacceptable for inclusion in the drug product if the level of expression of one or more genes selected from the group consisting of Gene Group 5 is not substantially identical to the level of expression by the same type of cells in the presence of the glatiramer acetate drug substance under the same conditions; vi) discarding the batch of the glatiramer acetate related drug substance as unacceptable for inclusion in the drug product if the level of expression of one or more genes selected from the group consisting of Gene Group 6, is not substantially identical to the level of expression by the same type of cells in the presence of the glatiramer acetate drug substance under the same conditions; vii) discarding the batch of the glatiramer acetate related drug substance as unacceptable for inclusion in the drug product if the level of expression of one or more genes selected from the group consisting of Gene Group 7, is downregulated or substantially identical to the level of expression by the same type of cells in the absence of the glatiramer acetate related drug substance under the same conditions; viii) discarding the batch of the glatiramer acetate related drug substance as unacceptable for inclusion in the drug product if the level of expression of one or more genes selected from the group consisting of Gene Group 8, is upregulated or substantially identical to the level of expression by the same type of cells in the absence of the glatiramer acetate related drug substance under the same conditions; ix) discarding the batch of the glatiramer acetate related drug substance as unacceptable for inclusion in the drug product if the level of expression of one or more genes selected from the group consisting of Gene Group 9 is not substantially identical to the level of expression by the same type of cells in the presence of the glatiramer acetate drug substance under the same conditions; x) discarding the batch of the glatiramer acetate related drug substance as unacceptable for inclusion in the drug product if the level of expression of one or more genes selected from the group consisting of Gene Group 10 is not substantially identical to the level of expression by the same type of cells in the presence of the glatiramer acetate drug substance under the same conditions; xi) discarding the batch of the glatiramer acetate related drug substance as unacceptable for inclusion in the drug product if the level of expression of one or more genes selected from the group consisting of Gene Group 11 is not substantially identical to the level of expression by the same type of cells in the presence of the glatiramer acetate drug substance under the same conditions; xii) discarding the batch of the glatiramer acetate related drug substance as unacceptable for inclusion in the drug product if the level of expression of one or more genes selected from the group consisting of Gene Group 12 is not substantially identical to the level of expression by the same type of cells in the presence of the glatiramer acetate drug substance under the same conditions; xiii) discarding the batch of the glatiramer acetate related drug substance as unacceptable for inclusion in the drug product if the level of expression of one or more genes selected from the group consisting of Gene Group 13 is not substantially identical to the level of expression by the same type of cells in the presence of the glatiramer acetate drug substance under the same conditions; xiv) discarding the batch of the glatiramer acetate related drug substance as unacceptable for inclusion in the drug product if the level of expression of one or more genes selected from the group consisting of Gene Group 14 is not substantially identical to the level of expression by the same type of cells in the presence of the glatiramer acetate drug substance under the same conditions; xv) discarding the batch of the glatiramer acetate related drug substance as unacceptable for inclusion in the drug product if the level of expression of one or more genes selected from the group consisting of Gene Group 15 is not substantially identical to the level of expression by the same type of cells in the presence of the glatiramer acetate drug substance under the same conditions; xvi) discarding the batch of the glatiramer acetate related drug substance as unacceptable for inclusion in the drug product if the level of expression of one or more genes selected from the group consisting of Gene Group 16 is not substantially identical to the level of expression by the same type of cells in the presence of the glatiramer acetate drug substance under the same conditions; xvii) discarding the batch of the glatiramer acetate related drug substance as unacceptable for inclusion in the drug product if the level of expression of one or more genes selected from the group consisting of Gene Group 17 is not substantially identical to the level of expression by the same type of cells in the presence of the glatiramer acetate drug substance under the same conditions; xviii) discarding the batch of the glatiramer acetate related drug substance as unacceptable for inclusion in the drug product if the level of expression of one or more genes selected from the group consisting of Gene Group 18 is not substantially identical to the level of expression by the same type of cells in the presence of the glatiramer acetate drug substance under the same conditions; xix) discarding the batch of the glatiramer acetate related drug substance as unacceptable for inclusion in the drug product if the level of expression of one or more genes selected from the group consisting of Gene Group 19 is not substantially identical to the level of expression by the same type of cells in the presence of the glatiramer acetate drug substance under the same conditions; xx) discarding the batch of the glatiramer acetate related drug substance as unacceptable for inclusion in the drug product if the level of expression of one or more genes selected from the group consisting of Gene Group 20 is not substantially identical to the level of expression by the same type of cells in the presence of the glatiramer acetate drug substance under the same conditions; xxi) discarding the batch of the glatiramer acetate related drug substance as unacceptable for inclusion in the drug product if the level of expression of one or more genes selected from the group consisting of Gene Group 21 is not substantially identical to the level of expression by the same type of cells in the presence of the glatiramer acetate drug substance under the same conditions; xxii) discarding the batch of the glatiramer acetate related drug substance as unacceptable for inclusion in the drug product if the level of expression of one or more genes selected from the group consisting of Gene Group 22 is not substantially identical to the level of expression by the same type of cells in the presence of the glatiramer acetate drug substance under the same conditions; xxiii) discarding the batch of the glatiramer acetate related drug substance as unacceptable for inclusion in the drug product if the level of expression of one or more genes selected from the group consisting of Gene Group 23 is not substantially identical to the level of expression by the same type of cells in the presence of the glatiramer acetate drug substance under the same conditions; xxiv) discarding the batch of the glatiramer acetate related drug substance as unacceptable for inclusion in the drug product if the level of expression of one or more genes selected from the group consisting of Gene Group 24 is not substantially identical to the level of expression by the same type of cells in the presence of the glatiramer acetate drug substance under the same conditions; xxv) discarding the batch of the glatiramer acetate related drug substance as unacceptable for inclusion in the drug product if the level of expression of one or more genes selected from the group consisting of Gene Group 25 is not substantially identical to the level of expression by the same type of cells in the presence of the glatiramer acetate drug substance under the same conditions; xxvi) discarding the batch of the glatiramer acetate related drug substance as unacceptable for inclusion in the drug product if the level of expression of one or more genes selected from the group consisting of Gene Group 26 is not substantially identical to the level of expression by the same type of cells in the presence of the glatiramer acetate drug substance under the same conditions; xxix) discarding the batch of the glatiramer acetate related drug substance as unacceptable for inclusion in the drug product if the level of expression of one or more genes selected from the group consisting of Gene Group 29 is not substantially identical to the level of expression by the same type of cells in the presence of the glatiramer acetate drug substance under the same conditions; or xxx) discarding the batch of the glatiramer acetate related drug substance as unacceptable for inclusion in the drug product if the level of expression of one or more genes selected from the group consisting of Gene Group 30 is not substantially identical to the level of expression by the same type of cells in the presence of the glatiramer acetate drug substance under the same conditions.

In a process for releasing a drug product comprising a glatiramer acetate related drug substance, the improvement comprising the steps of:
a) characterizing the glatiramer acetate related drug product according to the process of any one of the above characterization methods; and
b) i) discarding the batch of the glatiramer acetate related drug product as unacceptable for release if the level of expression of one or more genes selected from the group consisting of Gene Group 1 is substantially identical to the level of expression by the same type of cells in the absence of the glatiramer acetate related drug product under the same conditions; ii) discarding the batch of the glatiramer acetate related drug product as unacceptable for release if the level of expression of one or more genes selected from the group consisting of Gene Group 2 is not substantially identical to the level of expression by the same type of cells in the presence of the glatiramer acetate drug product under the same conditions; iii) discarding the batch of the glatiramer acetate related drug product as unacceptable for release if the level of expression of one or more genes selected from the group consisting of Gene Group 3 is not substantially identical to the level of expression by the same type of cells in the presence of the glatiramer acetate drug product under the same conditions; iv) discarding the batch of the glatiramer acetate related drug product as unacceptable for release if the level of expression of one or more genes selected from the group consisting of Gene Group 4 is not substantially identical to the level of expression by the same type of cells in the presence of the glatiramer acetate drug product under the same conditions; v) discarding the batch of the glatiramer acetate related drug product as unacceptable for release if the level of expression of one or more genes selected from the group consisting of Gene Group 5, is not substantially identical to the level of expression by the same type of cells in the presence of the glatiramer acetate drug product under the same conditions; vi) discarding the batch of the glatiramer acetate related drug product as unacceptable for release if the level of expression of one or more genes selected from the group consisting of Gene Group 6 is not substantially identical to the level of expression by the same type of cells in the presence of the glatiramer acetate drug product under the same conditions; vii) discarding the batch of the glatiramer acetate related drug product as unacceptable for release if the level of expression of one or more genes selected from the group consisting of Gene Group 7, is downregulated or substantially identical to the level of expression by the same type of cells in the absence of glatiramer acetate drug product under the same conditions; viii) discarding the batch of the glatiramer acetate related drug product as unacceptable for release if the level of expression of one or more genes selected from the group consisting of Gene Group 8, is upregulated or substantially identical to the level of expression by the same type of cells in the absence of glatiramer acetate drug product under the same conditions; ix) discarding the batch of the glatiramer acetate related drug product as unacceptable for release if the level of expression of one or more genes selected from the group consisting of Gene Group 9 is not substantially identical to the level of expression by the same type of cells in the presence of the glatiramer acetate drug product under the same conditions; x) discarding the batch of the glatiramer acetate related drug product as unacceptable for release if the level of expression of one or more genes selected from the group consisting of Gene Group 10 is not substantially identical to the level of expression by the same type of cells in the presence of the glatiramer acetate drug product under the same conditions; xi) discarding the batch of the glatiramer acetate related drug product as unacceptable for release if the level of expression of one or more genes selected from the group consisting of Gene Group 11 is not substantially identical to the level of expression by the same type of cells in the presence of the glatiramer acetate drug substance under the same conditions; xii) discarding the batch of the glatiramer acetate related drug product as unacceptable for release if the level of expression of one or more genes selected from the group consisting of Gene Group 12 is not substantially identical to the level of expression by the same type of cells in the presence of the glatiramer acetate drug substance under the same conditions; xiii) discarding the batch of the glatiramer acetate related drug product as unacceptable for release if the level of expression of one or more genes selected from the group consisting of Gene Group 13 is not substantially identical to the level of expression by the same type of cells in the presence of the glatiramer acetate drug substance under the same conditions; xiv) discarding the batch of the glatiramer acetate related drug product as unacceptable for release if the level of expression of one or more genes selected from the group consisting of Gene Group 14 is not substantially identical to the level of expression by the same type of cells in the presence of the glatiramer acetate drug substance under the same conditions; xv) discarding the batch of the glatiramer acetate related drug product as unacceptable for release if the level of expression of one or more genes selected from the group consisting of Gene Group 15 is not substantially identical to the level of expression by the same type of cells in the presence of the glatiramer acetate drug substance under the same conditions; xvi) discarding the batch of the glatiramer acetate related drug product as unacceptable for release if the level of expression of one or more genes selected from the group consisting of Gene Group 16 is not substantially identical to the level of expression by the same type of cells in the presence of the glatiramer acetate drug substance under the same conditions; xvii) discarding the batch of the glatiramer acetate related drug product as unacceptable for release if the level of expression of one or more genes selected from the group consisting of Gene Group 17 is not substantially identical to the level of expression by the same type of cells in the presence of the glatiramer acetate drug substance under the same conditions; xviii) discarding the batch of the glatiramer acetate related drug product as unacceptable for release if the level of expression of one or more genes selected from the group consisting of Gene Group 18 is not substantially identical to the level of expression by the same type of cells in the presence of the glatiramer acetate drug substance under the same conditions; xix) discarding the batch of the glatiramer acetate related drug product as unacceptable for release if the level of expression of one or more genes selected from the group consisting of Gene Group 19 is not substantially identical to the level of expression by the same type of cells in the presence of the glatiramer acetate drug substance under the same conditions; xx) discarding the batch of the glatiramer acetate related drug product as unacceptable for release if the level of expression of one or more genes selected from the group consisting of Gene Group 20 is not substantially identical to the level of expression by the same type of cells in the presence of the glatiramer acetate drug substance under the same conditions; xxi) discarding the batch of the glatiramer acetate related drug product as unacceptable for release if the level of expression of one or more genes selected from the group consisting of Gene Group 21 is not substantially identical to the level of expression by the same type of cells in the presence of the glatiramer acetate drug substance under the same conditions; xxii) discarding the batch of the glatiramer acetate related drug product as unacceptable for release if the level of expression of one or more genes selected from the group consisting of Gene Group 22 is not substantially identical to the level of expression by the same type of cells in the presence of the glatiramer acetate drug substance under the same conditions; xxiii) discarding the batch of the glatiramer acetate related drug product as unacceptable for release if the level of expression of one or more genes selected from the group consisting of Gene Group 23 is not substantially identical to the level of expression by the same type of cells in the presence of the glatiramer acetate drug substance under the same conditions; xxiv) discarding the batch of the glatiramer acetate related drug product as unacceptable for release if the level of expression of one or more genes selected from the group consisting of Gene Group 24 is not substantially identical to the level of expression by the same type of cells in the presence of the glatiramer acetate drug substance under the same conditions; xxv) discarding the batch of the glatiramer acetate related drug product as unacceptable for release if the level of expression of one or more genes selected from the group consisting of Gene Group 25 is not substantially identical to the level of expression by the same type of cells in the presence of the glatiramer acetate drug substance under the same conditions; xxvi) discarding the batch of the glatiramer acetate related drug product as unacceptable for release if the level of expression of one or more genes selected from the group consisting of Gene Group 26 is not substantially identical to the level of expression by the same type of cells in the presence of the glatiramer acetate drug substance under the same conditions; xxix) discarding the batch of the glatiramer acetate related drug product as unacceptable for release if the level of expression of one or more genes selected from the group consisting of Gene Group 29 is not substantially identical to the level of expression by the same type of cells in the presence of the glatiramer acetate drug substance under the same conditions; or xxx) discarding the batch of the glatiramer acetate related drug product as unacceptable for release if the level of expression of one or more genes selected from the group consisting of Gene Group 20 is not substantially identical to the level of expression by the same type of cells in the presence of the glatiramer acetate drug substance under the same conditions.

In one or more embodiments of the present invention, in a process for identifying suboptimal activity of a glatiramer acetate related drug substance or drug product comprising the steps of:
a) administering a glatiramer acetate related drug substance or drug product to a mammal;
b) characterizing the glatiramer acetate related drug substance or drug product according to the process of any one of the above characterization methods; and
c) i) identifying the glatiramer acetate related drug substance or drug product as causing a suboptimal activity if the level of expression of one or more genes selected from the group consisting of Gene Group 1 is substantially identical to the level of expression by the same type of cells in the absence of the glatiramer acetate related drug substance or drug product under the same conditions; ii) identifying the glatiramer acetate related drug substance or drug product as causing a suboptimal activity if the level of expression of one or more genes selected from the group consisting of Gene Group 2 is not substantially identical to the level of expression by the same type of cells in the presence of the glatiramer acetate related drug substance or drug product under the same conditions; iii) identifying the glatiramer acetate related drug substance or drug product as causing a suboptimal activity if the level of expression of one or more genes selected from the group consisting of Gene Group 3 is not substantially identical to the level of expression by the same type of cells in the presence of the glatiramer acetate related drug substance or drug product under the same conditions; iv) identifying the glatiramer acetate related drug substance or drug product as causing a suboptimal activity if the level of expression of one or more genes selected from the group consisting of Gene Group 4 is not substantially identical to the level of expression by the same type of cells in the presence of the glatiramer acetate related drug substance or drug product under the same conditions; v) identifying the glatiramer acetate related drug substance or drug product as causing a suboptimal activity if the level of expression of one or more genes selected from the group consisting of Gene Group 5 is not substantially identical to the level of expression by the same type of cells in the presence of the glatiramer acetate drug substance or drug product under the same conditions; vi) identifying the glatiramer acetate related drug substance or drug product as causing a suboptimal activity if the level of expression of one or more genes selected from the group consisting of Gene Group 6, is not substantially identical to the level of expression by the same type of cells in the presence of the glatiramer acetate related drug substance or drug product under the same conditions; vii) identifying the glatiramer acetate related drug substance or drug product as causing a suboptimal activity if the level of expression of one or more genes selected from the group consisting of Gene Group 7, is downregulated or substantially identical to the level of expression by the same type of cells in the absence of the glatiramer acetate related drug substance or drug product under the same conditions; viii) identifying the glatiramer acetate related drug substance or drug product as causing a suboptimal activity if the level of expression of one or more genes selected from the group consisting of Gene Group 8, is upregulated or substantially identical to the level of expression by the same type of cells in the absence of the glatiramer acetate related drug substance or drug product under the same conditions; ix) identifying the glatiramer acetate related drug substance or drug product as causing a suboptimal activity if the level of expression of one or more genes selected from the group consisting of Gene Group 9 is not substantially identical to the level of expression by the same type of cells in the presence of the glatiramer acetate drug substance or drug product under the same conditions; x) identifying the glatiramer acetate related drug substance or drug product as causing a suboptimal activity if the level of expression of one or more genes selected from the group consisting of Gene Group 10 is not substantially identical to the level of expression by the same type of cells in the presence of the glatiramer acetate drug substance or drug product under the same conditions; xi) identifying the glatiramer acetate related drug substance or drug product as causing a suboptimal activity if the level of expression of one or more genes selected from the group consisting of Gene Group 11 is not substantially identical to the level of expression by the same type of cells in the presence of the glatiramer acetate drug substance under the same conditions; xii) identifying the glatiramer acetate related drug substance or drug product as causing a suboptimal activity if the level of expression of one or more genes selected from the group consisting of Gene Group 12 is not substantially identical to the level of expression by the same type of cells in the presence of the glatiramer acetate drug substance under the same conditions; xiii) identifying the glatiramer acetate related drug substance or drug product as causing a suboptimal activity if the level of expression of one or more genes selected from the group consisting of Gene Group 13 is not substantially identical to the level of expression by the same type of cells in the presence of the glatiramer acetate drug substance under the same conditions; xiv) identifying the glatiramer acetate related drug substance or drug product as causing a suboptimal activity if the level of expression of one or more genes selected from the group consisting of Gene Group 14 is not substantially identical to the level of expression by the same type of cells in the presence of the glatiramer acetate drug substance under the same conditions; xv) identifying the glatiramer acetate related drug substance or drug product as causing a suboptimal activity if the level of expression of one or more genes selected from the group consisting of Gene Group 15 is not substantially identical to the level of expression by the same type of cells in the presence of the glatiramer acetate drug substance under the same conditions; xvi) identifying the glatiramer acetate related drug substance or drug product as causing a suboptimal activity if the level of expression of one or more genes selected from the group consisting of Gene Group 16 is not substantially identical to the level of expression by the same type of cells in the presence of the glatiramer acetate drug substance under the same conditions; xvii) identifying the glatiramer acetate related drug substance or drug product as causing a suboptimal activity if the level of expression of one or more genes selected from the group consisting of Gene Group 17 is not substantially identical to the level of expression by the same type of cells in the presence of the glatiramer acetate drug substance under the same conditions; xviii) identifying the glatiramer acetate related drug substance or drug product as causing a suboptimal activity if the level of expression of one or more genes selected from the group consisting of Gene Group 18 is not substantially identical to the level of expression by the same type of cells in the presence of the glatiramer acetate drug substance under the same conditions; xix) identifying the glatiramer acetate related drug substance or drug product as causing a suboptimal activity if the level of expression of one or more genes selected from the group consisting of Gene Group 19 is not substantially identical to the level of expression by the same type of cells in the presence of the glatiramer acetate drug substance under the same conditions; xx) identifying the glatiramer acetate related drug substance or drug product as causing a suboptimal activity if the level of expression of one or more genes selected from the group consisting of Gene Group 20 is not substantially identical to the level of expression by the same type of cells in the presence of the glatiramer acetate drug substance under the same conditions; xxi) identifying the glatiramer acetate related drug substance or drug product as causing a suboptimal activity if the level of expression of one or more genes selected from the group consisting of Gene Group 21 is not substantially identical to the level of expression by the same type of cells in the presence of the glatiramer acetate drug substance under the same conditions; xxii) identifying the glatiramer acetate related drug substance or drug product as causing a suboptimal activity if the level of expression of one or more genes selected from the group consisting of Gene Group 22 is not substantially identical to the level of expression by the same type of cells in the presence of the glatiramer acetate drug substance under the same conditions; xxiii) identifying the glatiramer acetate related drug substance or drug product as causing a suboptimal activity if the level of expression of one or more genes selected from the group consisting of Gene Group 23 is not substantially identical to the level of expression by the same type of cells in the presence of the glatiramer acetate drug substance under the same conditions; xxiv) identifying the glatiramer acetate related drug substance or drug product as causing a suboptimal activity if the level of expression of one or more genes selected from the group consisting of Gene Group 24 is not substantially identical to the level of expression by the same type of cells in the presence of the glatiramer acetate drug substance under the same conditions; xxv) identifying the glatiramer acetate related drug substance or drug product as causing a suboptimal activity if the level of expression of one or more genes selected from the group consisting of Gene Group 25 is not substantially identical to the level of expression by the same type of cells in the presence of the glatiramer acetate drug substance under the same conditions; xxvi) identifying the glatiramer acetate related drug substance or drug product as causing a suboptimal activity if the level of expression of one or more genes selected from the group consisting of Gene Group 26 is not substantially identical to the level of expression by the same type of cells in the presence of the glatiramer acetate drug substance under the same conditions; xxix) identifying the glatiramer acetate related drug substance or drug product as causing a suboptimal activity if the level of expression of one or more genes selected from the group consisting of Gene Group 29 is not substantially identical to the level of expression by the same type of cells in the presence of the glatiramer acetate drug substance under the same conditions; or xxx) identifying the glatiramer acetate related drug substance or drug product as causing a suboptimal activity if the level of expression of one or more genes selected from the group consisting of Gene Group 30 is not substantially identical to the level of expression by the same type of cells in the presence of the glatiramer acetate drug substance under the same conditions.

The Additional Embodiments described above can be readily applied and practiced with features of Embodiments of the Invention described in the section preceeding the Additional Embodiments.

For example, in a process for producing a drug product comprising a glatiramer acetate related drug substance, where the batch of the glatiramer acetate related drug substance is included in the drug product if the level of expression of one or more genes selected from one or more Gene Groups is substantially upregulated relative to the level of expression by the same type of cells in the absence of the glatiramer acetate related drug substance under the same conditions, then conversely the batch of the glatiramer acetate related drug substance is discarded if the level of expression of one or more genes selected from one or more Gene Groups is not substantially upregulated relative to the level of expression by the same type of cells in the absence of the glatiramer acetate related drug substance under the same conditions as unacceptable for inclusion in the drug product.

### DEFINITIONS

As used herein, a “naïve human” is a human that has not been treated with any multiple sclerosis drug.

As used herein, a “glatiramoid naïve human” is a human that has not been treated with any glatiramoid drug. A glatiramoid naïve human could have been treated with another multiple sclerosis drug.

As used herein, “in the blood of” is represented by peripheral blood mononuclear cells (PBMCs), lymphocytes, monocytes, macrophages, basophils, dendritic cells or other cells derived from a mammal's blood.

As used herein, a “reference standard” is a sample or value which serves as a point of comparison for another sample or value which differs from the reference standard with respect to one or more variables. With specific regard to a human, a “reference standard” is a value or range of values that characterizes a defined population in a defined state of health. For example, a reference standard can characterize a healthy human or a human afflicted with multiple sclerosis, and when the human is afflicted with multiple sclerosis the human can be naïve or having received glatiramer acetate drug substance.

As used herein, the term “glatiramer acetate related drug substance” (GARDS) is intended to include include polypeptides with a predetermined sequence as well as mixtures of polypeptides assembled from the four amino acids glutamic acid (E), alanine (A), lysine (K), and tyrosine (Y); from any three of the amino acids Y, E, A and K, i.e. YAK, YEK, YEA or EAK; or from three of the amino acids Y, E, A and K and a fourth amino acid. Examples of glatiramer acetate related polypeptides are disclosed in U.S. Pat. Nos. 6,514,938 A1, 7,299,172 B2, 7,560,100 and 7,655,221 B2 and U.S. Patent Application Publication No. US 2009-0191173 A1, the disclosures of which are hereby incorporated by reference in their entireties. Glatiramer acetate related substances include glatiramoids.

As used herein, a “glatiramer acetate related drug product” (GARDP) contains a glatiramer acetate related drug substance.

As used herein, a “glatiramer acetate related drug substance or drug product” is a glatiramer acetate related drug substance or a glatiramer acetate related drug product.

As used herein a “glatiramoid” is a complex mixture of synthetic proteins and polypeptides of varying sizes assembled from four naturally occurring amino acids: L-glutamic acid, L-alanine, L-lysine, and L-tyrosine, in a defined molar ratio. Examples of glatiramoids include glatiramer acetate drug substance (e.g. Copaxone®) as well as glatiramoids other than Copaxone®, e.g. GA-Natco.

As used herein, a “glatiramer acetate drug substance” (GADS) is glatiramer acetate produced by Teva Pharmaceutical Industries, Ltd. and is the active ingredient in a glatiramer acetate drug product.

As used herein, a “glatiramer acetate drug product” (GADP) contains a glatiramer acetate drug substance produced by Teva Pharmaceutical Industries, Ltd. which consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively, and has an average molecular weight of 5,000-9,000 daltons. (8) Copaxone® is a glatiramer acetate drug product.

As used herein, a “glatiramer acetate drug substance or drug product” is a glatiramer acetate drug substance or a glatiramer acetate drug product.

As used herein a “glatiramer acetate reference standard” is or contains the drug substance found in a glatiramer acetate drug product.

As used herein “suboptimal activity” refers to a negative response or to a response which is less than the response to glatiramer acetate drug substance or glatiramer acetate drug product produced by Teva Pharmaceutical Industries, Ltd.

As used herein, “release” of a drug product refers to making the product available to consumers.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

As used herein, “hematological cell” comprises neutrophils, erythrocytes, basophils, monocytes, eosinophils, platelets, lymphocytes, and splenocytes.

As used herein, an “array of testing” for a glatiramer acetate related drug substance or drug product includes, but is not limited to, any analytical method test such as in vitro tests or molecular weight tests, biological assays such as the ex vivo tests and clinical efficacy tests which characterize the GARDS or GARDP, or clinical trials. Examples of testing for a glatiramer acetate related drug substance or drug product are disclosed in U.S. Patent Application Publication Nos. US 2012-0309671 and US 2011-0230413, and in PCT International Application Publication Nos. WO 2000/018794, WO 2012/051106, WO 2013/055683, WO 2014/058976, the disclosures of which are hereby incorporated by reference in their entireties.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

All combinations of the various elements described herein are within the scope of the invention.

It is understood that where “at least one” or “one or more” is recited along with a list, then 1, 2, 3, 4 . . . or all members of that list are disclosed in every combination. For example, in a group of 43 genes, “at least one” and “one or more” is a disclosure of one gene, two genes, three genes, etc., in any combination, up to the forty three genes.

As used herein, “characterization” or “characterizing” is understood to include obtaining information which was produced in the same location or country, or a different location or country from where any remaining steps of the method are performed.

As used herein, processes of producing a glatiramer acetate related drug substance or drug product are known in the art. Examples of such manufacturing processes are disclosed in U.S. Pat. No. 5,800,808, and in PCT International Application Publication Nos. WO 2005/032553, WO 2005/032395, WO 1999/22402, the disclosures of which are hereby incorporated by reference in their entireties.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Methods

The null hypothesis in traditional gene-expression studies, including Teva's studies with the glatiramoids, is that there are no significant gene expression differences induced between the treatments. As such, the expectation is that regardless of the biological system used for testing, genes would show no statistically significant, nor biologically meaningful differences among the various treatments. Only in cases where the treatments induce significant observable effects, genes differentially expressed between treatments will pass the stringent statistical tests, and false discovery rate (FDR) correction for multiple hypotheses (Benjamini and Hochberg, 1995). These stringent requirements were imposed a priori across all tests to ensure robustness of results and minimizing of spurious findings. Such statistically significant differences, if biologically meaningful (i.e., related to the disease biology or any of the drug's known or putative targets and downstream pathways), warrant further studies, as two drugs that have identical activities in biological systems should not induce statistically observable and biologically enriched differences when compared against each other.

These studies were not designed to establish a particular set of genes in a specific model system as a panel to evaluate sameness between differently manufactured glatiramoids. Instead, these were designed to assess the degree of similarity in the impact of two glatiramoids on relevant biological pathways. The application of robust methodology (high number of replicates, conditions and time-points, where relevant) was aimed to describe pathways changed by different treatments out of the entire milieu of genomic patterns. The results obtained across the tested experimental models revealed statistically significant differences between drugs, which were intended to be similar and to perform the same function, despite stringent statistical threshold requirements. This was noteworthy particularly in genes highly relevant to disease processes and drug response mechanisms. In addition, the differences observed revealed a complex interplay between immunological pathways, such that some differences were common to multiple systems, while many others were dependent on the specific model system (for example, some key genes modulated in T cells were not the same as in monocytes). This is not surprising for a process that involves multifaceted interactions between many immune system components, and is also exemplified in experimental studies of Copaxone®'s mechanism of action. Thus, no single model system, characterization method, or set of genes tested was sufficient to comprehensively capture the differences between the drugs. These observations indicate a need for in-depth investigation of comparative gene expression profiles in several relevant pre-clinical systems as key indicators of similarity and/or sameness between generic candidates and the original drug within the context of Non-Biological Complex Drugs (NBCD). Ideally, the concordance between high-resolution physicochemical measures (e.g. ion motility mass spectrometry, IMMS), gene expression profiling and clinical trials would allow a more definitive assessment of equivalence in terms of patient benefit and safety.

### Mice

All experimental procedures conformed to accepted ethical standards for use of animals in research and were in accordance with Committee for the Care and Use of Experimental Animals guidelines and approved by the Teva Institutional Animal Care and Use Committee.

### Preparation of Mouse Spleen Cell Cultures

All experimental procedures conformed to accepted ethical standards for use of animals in research and were in accordance with Committee for the Care and Use of Experimental Animals guidelines and approved by the Teva Institutional Animal Care and Use Committee.

### Mouse Splenocytes: Gene Expression Analysis

In the first of the genomic studies, 8- to 12-week old female mice (N=8) of the (Balb/c x SJL) F1 variety (Harlan, Israel) were injected subcutaneously with glatiramer acetate (GA) in order to induce specific GA-reactive T cells as would be present in a Copaxone® treated patient.

After 3 days, the mice were sacrificed and cells from their spleens (splenocytes) were isolated because such cells are representative of, and commonly utilized as a gold standard to study the immune system. The aqueous activator samples, mannitol (the non-active excipient in Copaxone® and all other marketed proposed generics) and medium were used as negative controls.

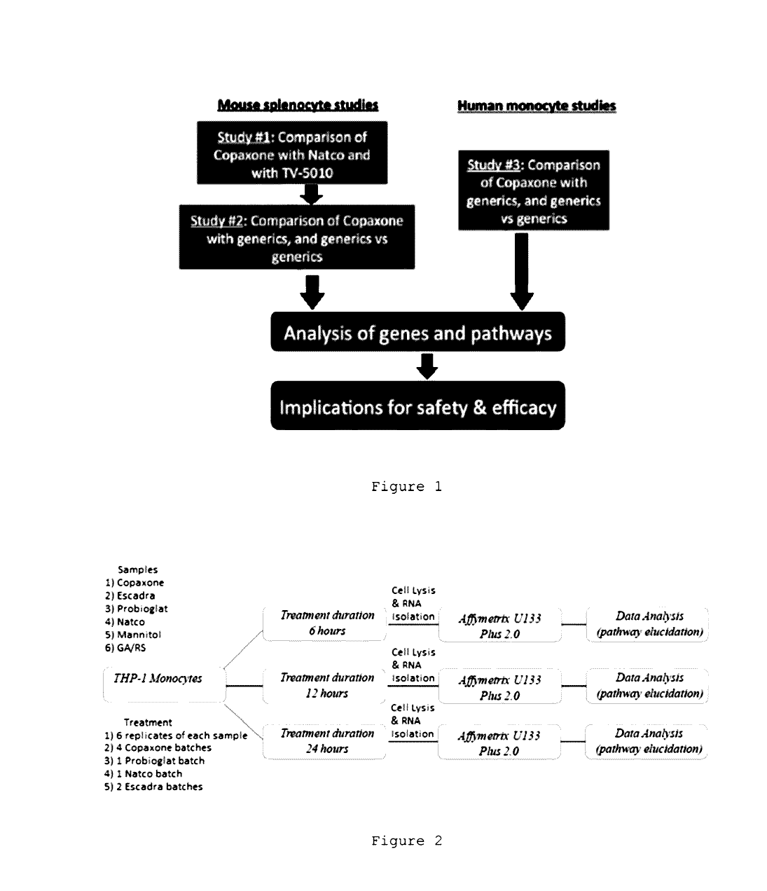

Given the robustness of the model established in our first mouse splenocyte study, we sought to expand the scope of the investigation using the exact same study design, but with an expanded set of treatments (FIG. 1). In addition to comparing Copaxone® to a purported foreign generic version, we also sought to compare purported generics from various manufacturers to each other. Using the mouse splenocyte procedure described herein, we generated expression data in response to activation with proposed generic glatiramer acetate samples from Escadra (Argentina), Probioglat (Mexico; Probiomed), and Hangzhou (China: API). A total of 21 samples of Copaxone® from 21 different batches were used, along with 10 samples from 2 lots of Escadra, 12 samples from 4 batches of Natco, 4 samples from 1 batch of Probioglat, 5 samples of Hangzhou from 2 batches, 12 samples of GA-RS from 1 batch, and 8 samples of medium.

### Mouse Splenocytes: RNA Isolation and Microarray Expression Profiling

Extraction of total RNA from activated splenocytes was extracted using PerfectPure RNA Cultures CEKK kit 50 (5Prime GmbH, Hamburg, Germany) and following the manufacturer's instructions. RNA quality was assessed using the absorbance ratio at 260/280 nm and gel electrophoresis (Experion, Bio-Rad, Hercules, Calif., USA). Total RNA extracted from samples was hybridized to Illumina Mouse WG-6V2 microarray chips containing more than 45,200 transcripts.

Samples were randomized on the chips to avoid batch effects. Illumina's BeadStudio software was utilized for image processing, quantification of signal intensity per bead, and background signal correction. Multiple probes were analyzed for a given gene were averaged and batch correction was conducted using Partek. Further analysis was conducted individually within the context of known pathways, and batch correction was conducted using ComBat, as implemented in the SVA R package sva: Surrogate Variable Analysis. Available: www.bioconductor.org/packages/release/bioc/html/sva.html). Briefly, ComBat is an empirical Bayesian approach utilizing location and scale metrics across several genes to adjust for batch effects in datasets, even datasets containing small sample sizes. Treatment labels were added as covariates to the batch correction in order to preserve relevant treatment effects. Principal Component Analysis (a multivariate approach) showed that the main effect in the first principal component remained due to treatment effects after batch correction.

Mouse Splenocytes: Gene Expression Analysis—Polimunol versus Copaxone® Gene Expression Studies

### Mice

All experimental procedures conformed to accepted ethical standards for use of animals in research and were in accordance with Committee for the Care and Use of Experimental Animals guidelines and approved by the Teva Institutional Animal Care and Use Committee. For these experiments, 8- to 12-week-old female (Balb/c X SJL) F1 mice (Janvier, France) were purchased. Mice were kept at 21±3° C.; the relative humidity was 30-70%, the light/dark cycle was 12/12 h. Animals were maintained on a standard rodent pellet diet and sterile filtered tap water available ad libitum.

Preparation of Mouse Spleen Cell Cultures

To stimulate induction of GA-reactive T cells, twelve mice were injected with 100 μL of a 2.5 mg/mL solution of GA reference standard (GA-RS) in phosphate-buffered saline (250 μg GARS per mouse). GA-RS (Teva) is a selected GA drug substance batch defined as the standard batch and used as a reference in quality control release tests of all Copaxone® batches. Another group of twelve mice were injected with 100 μL of a 2.5 mg/mL solution of Polimunol in phosphate-buffered saline (250 μg Polimunol per mouse). Polimunol is a proposed generic manufactured a company other than Teva. Mice were housed for 3 days after immunization; mice were then sacrificed and their spleens were aseptically removed and placed on ice in RPMI 1640. A single cell suspension was prepared. After red blood cells lysis, splenocytes from the same immunization group were pulled and resuspended to a final concentration of 10×106 cells/mL in defined cell culture media (DCCM1) (Biological Industries, Beit Haemek, Israel) (96.7% v/v) enriched with L-glutamine 2 mM (1% v/v), MEM Non-Essential Amino Acids 2 mM (1% v/v), sodium pyruvate 1 mM (1% v/v), antibiotic/antimycotic solution (0.2% v/v) and 2-mercaptoethanol 50 mM (0.1% v/v).

### In Vitro Cell Activation

Splenocytes were treated with activator samples diluted in medium (125 μL per well of 80 μg/mL solutions, final concentration in the well: 40 μg/mL) of: i) 3 different batches of GA drug product manufactured by Teva ii) one batch of proposed generic (Polimunol). Polimunol is a product marketed as generic GA and manufactured by a company other than Teva (i.e. Synthon). The activator samples, mannitol (the nonactive excipient in Copaxone®), and medium were added to 96-well tissue culture plates (three wells per sample). Splenocytes (125 μL 10×10⁶ SPL cell/mL suspension) were added to the activator solutions. Each activator sample was loaded in two different plates. One for the cells from mice immunized with GA and one for cells from mice immunized with proposed generic. Plates were incubated for 24 h at 37° C. Cells were collected from the wells and were centrifuged at 300 g for 5 minutes. Supernatants were aspirated and cell pellets were resuspended in RLT buffer (from RNeasy mini kit of Qiagen, Cat #74106) for cell lysis. The cell lysates were centrifuged and supernatants were collected and frozen at −70° C. Samples were sent for further processing.

Analysis Methods

For additional detail on experimental methods used in mouse studies, please refer to publications [Bakshi et al; Towfic et al]. Note that in this study, mice were immunized with either Copaxone® or Polimunol, and subsequently splenocytes were isolated and treated ex vivo with Copaxone® or Polimunol. RNA was extracted and expression profiled across the entire genome using the Affymetrix Mouse Genome 430 2 chip. Three lots of Copaxone®, and one lot of Polimunol were comparatively tested in six replicates each.

### Outlier Identification and Normalization

Outlier samples were identified using the R package ArrayQCMetrics and excluded from further data processing steps. A sample was considered an outlier if it failed more than half of the included tests either before or after RMA normalization. Data were RMA normalized using the Affy R package.

### Batch Correction

Correction for batch variation was performed using ComBat as described above (THP-1 methods). Date of microarray experiment was used as batch, and the combination of treatment and immunization was used as covariate. Principal Component Analysis (a multivariate approach) showed that the main effect in the first principal component remained due to treatment effects after batch correction.

### Differential Expression Analysis

Differentially expressed probesets were identified across conditions using linear models for microarray data (LIMMA; Smyth, G. K. (2004)), a standard R Bioconductor package. Linear models and empirical Bayes methods for assessing differential expression in microarray experiments. Statistical Applications in Genetics and Molecular Biology 3, No. 1, Article 3). To compare GA and purported generic, comparisons were corrected to compare each treatment relative to mannitol control (e.g., [GA vs mannitol] was compared via LIMMA to [Polimunol vs mannitol]). Probesets were filtered by MAS5 calls of presence on the chip (to be considered present, a probeset was required to have on average a call of present or marginal across samples). Probesets were mapped to genes using the annotation available for the Mouse 430 2 chip from Affymetrix. Unless otherwise noted, FDR adjusted p values reported for genes represent the lowest FDR adjusted p value for present probesets for that gene.

### Pathway Enrichment Analysis

Upregulated and downregulated probesets were analyzed separately for pathway enrichment, using DAVID (Huang et al, Nucleic Acids Res 2009). Pathway enrichment results were visualized using volcano plots, plotting either −log adjusted p values or untransformed adjusted p values versus enrichment scores for the pathways. For comparisons between branded GA and Polimunol, upregulated or downregulated probesets with FDR-adjusted p values<0.05 and fold changes (FC) with absolute value greater than 1.2 were used for pathway enrichment. For comparisons between branded GA and mannitol, upregulated or downregulated probesets with FDR-adjusted p values<0.05 and FC with absolute value greater than 2 were used for pathway enrichment. DAVID runs were conducted in December 2014 and January 2015.

### Human Monocyte Cell Line: Gene Expression Analysis

THP-1 human monocytes (TIB-202) were purchased from ATCC®. Cells were maintained in recommended RPMI-1640 media containing FCS, L-Glutamine, Sodium Pyruvate, D-Glucose, HEPES, and 2-mercaptoethanol at 37° C. and 5% CO2. Prior to treatment cells were passed and plated in a 6-well plate at a concentration of 1.0×10⁶ cells/mL. Cells were allowed to recover for four hours after passage (prior to treatment). Using a predetermined non toxic concentration the cells were spiked with 50 μg/mL of either Copaxone®, GA/RS, Escadra, Probioglat, and Natco, and 100 μg/mL mannitol was spiked as a negative control (vehicle). Each sample was analyzed in six replicates. Cells were treated for 6, 12, and 24 hours. Following treatment time cells were lysed using a Qiagen® based lysis kit. Prior to RNA isolation samples were randomized in order to avoid batch effect. Total RNA was isolated using Qiagen® RNeasy kit. RNA quality was assessed by determining the absorbance ratio 260/280 nm as well as electrophoresis bioanalyzer with 260/280 ratio of 1.9-2.1 and RIN of above 9 were deemed acceptable. Gene expression was measured using Affymetrix® U133 plus 2.0 format. Sample processing was executed according to established manufacturer protocols. The scheme in (FIG. 2) illustrates the general experimental outline.

### Human Monocyte Cell Line: RNA Isolation and Microarray Expression Profiling

RNA from THP-1 cells was extracted and expression profiled across the entire genome using the Affymetrix Human Genome U133 Plus 2.0 chip, interrogating a total of over 47,000 transcripts. Four batches of Copaxone® and one batch of Probioglat were comparatively tested in six biological replicates each. Key identified genes were independently evaluated for level of gene expression by quantitative Real-Time PCR of samples collected in the same experiments.

### Methods for Studies Using MS Patient Data

Patient studies utilized a subset of MS patients treated with Copaxone® from FORTE clinical trial with time-series PBMC expression profiling. Timepoints of 0, 1, 2, 3 months were used. The study included 9 patients for a total of 36 samples.

### Repeated-Measures ANOVA

ANOVA was run taking into account multiple timepoints from each patient. Variability within patients across timepoints was taken into account in the p value calculation.

qRT-PCR Analysis

Key genes identified by differential expression analysis were assayed using qRT-PCR. RNA was utilized from each of 6 biological samples for each treatment (Copaxone® and Probioglat) and 15 technical replicates were performed for each sample (a total of 90 observations per transcript per treatment). Since three Copaxone® batches and one Probioglat batch were available, a total of 360 observations from each transcript were evaluated. To evaluate the data, the 2^{-ΔΔct} approximation was utilized with GAPDH as reference transcript and vehicle control (mannitol) as calibrator. A one-sided t-test with unequal variance was used to compare the RNA expression from the two treatments.

Human Monocyte Cell Line: Gene Expression Analysis—Polimunol versus Copaxone® Gene Expression Studies

### Experimental Design

Cells from a human monocyte cell line (THP-1) were stimulated with either branded glatiramer acetate—Copaxone®, or the purported generic Polimunol, or vehicle control (mannitol) for 6 hours. RNA was extracted and expression profiled in a blinded fashion across the entire genome, using the Affymetrix Human Genome U133 Plus 2.0 chip, interrogating a total of over 47,000 transcripts. Three lots of Copaxone® (148, 164, 166) and one lot of Polimunol (XBN) were comparatively tested in six replicates each. This entire experiment was performed independently twice, using an identical study design, reagents and compounds by the same technicians on two separate days; resulting in twelve replicates total per condition.

### A Priori Power Analysis

Using the R statistical package ssize.fdr, power calculations were performed to determine the number of samples needed to detect differentially expressed genes with a fold-change between treatments of as low as 1.3 with 80% power. Based on the results of these power calculations, the experiment was designed to include six replicates of each condition. The order in which the samples were processed was randomized with respect to treatment and stimulation time in order to avoid creating confounding batch effects.

### Outlier Identification and Normalization

Outlier samples were identified using the R package ArrayQCMetrics and excluded from further data processing steps. A sample was considered an outlier if it failed more than half of the included tests either before or after RMA normalization. Data were RMA normalized using the Affy R package.

### Batch Correction

Correction for batch variation was performed using ComBat (Johnson et al, Biostat, 2007), as implemented in the SVA R package (Leek J T, Johnson W E, Parker H S, Jaffe A E, Storey J D (2013) sva: Surrogate Variable Analysis. Available: www.bioconductor.org/packages/release/bioc/html/sva.html). Briefly, ComBat is an empirical Bayesian approach utilizing location and scale metrics across several genes to adjust for batch effects in datasets, even datasets containing small sample sizes. Date of experiment was utilized as batch. Treatment labels were added as covariates to the batch correction in order to preserve relevant treatment effects. Principal Component Analysis (a multivariate approach) showed that the main effect in the first principal component remained due to treatment effects after batch correction.

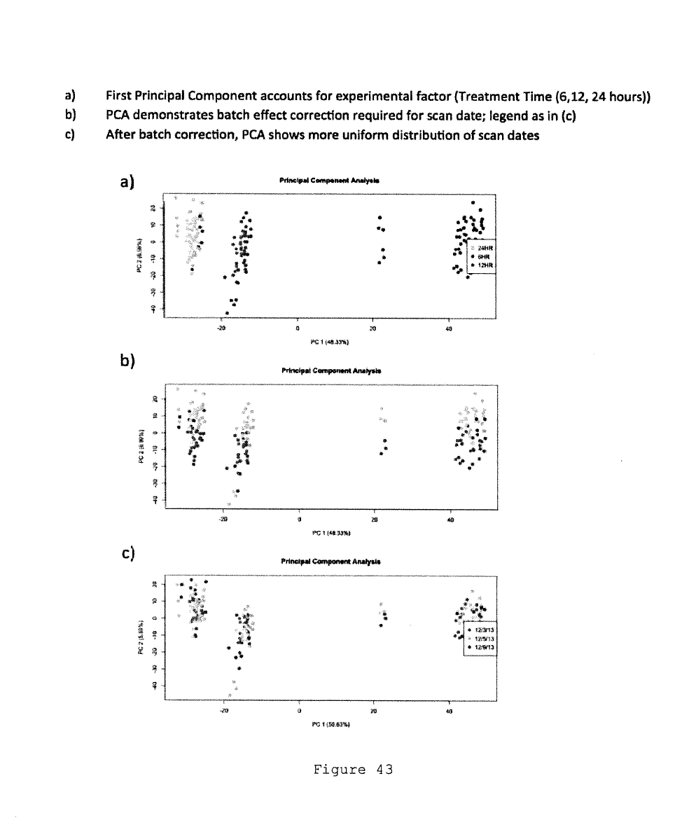

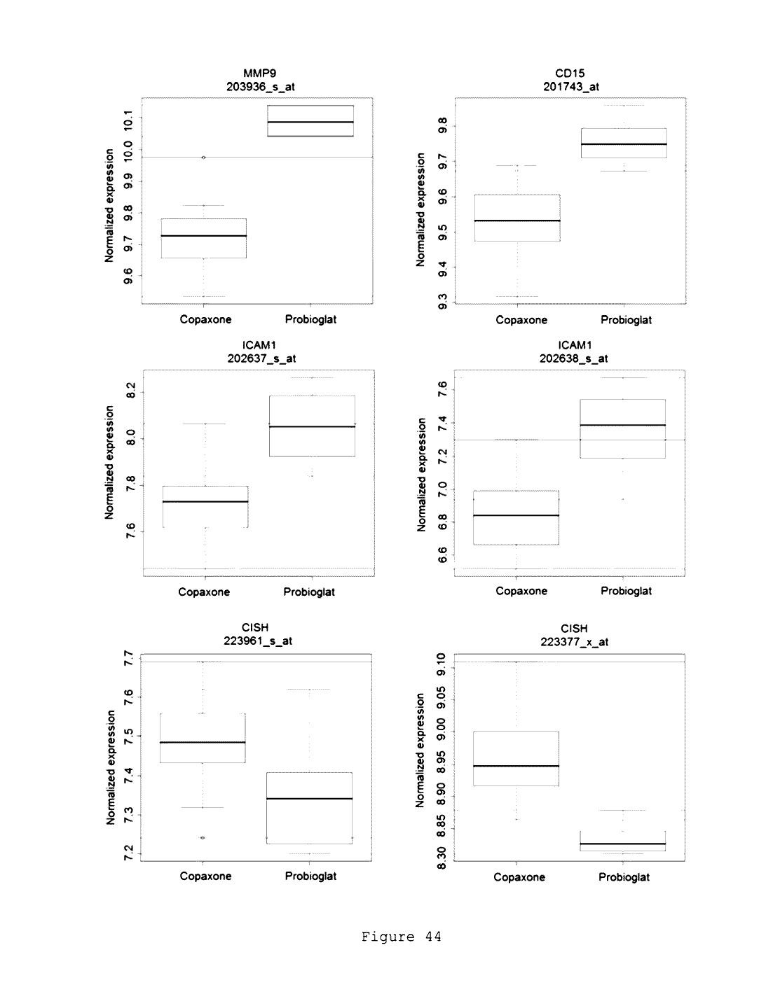

Treatment labels were added as covariates to the batch correction to preserve relevant treatment effects. Principal Component Analysis showed the main effect in PC1 remained due to treatment effects after batch correction (FIG. 43).

### Differential Expression Analysis

Differentially expressed probesets were identified across conditions using linear models for microarray data (LIMMA; Smyth, G. K. (2004)), a standard R Bioconductor package. Linear models and empirical Bayes methods for assessing differential expression in microarray experiments. Statistical Applications in Genetics and Molecular Biology 3, No. 1, Article 3). To compare Copaxone® and a purported generic, comparisons were corrected to compare each treatment relative to mannitol control (e.g., [GA vs mannitol] was compared via LIMMA to [purported generic vs mannitol]). For use in pathway analyses, probesets were filtered by MAS5 calls of presence on the chip for the relevant samples in the comparison (e.g., to be considered present, a probeset was required to have on average a call of present or marginal across samples). An additional QC step was performed to remove probesets determined to be highly variable between multiple THP-1 datasets, as follows: a probeset was deemed highly variable if across three THP-1 studies to date, that probeset was observed to be upregulated, downregulated, and not modulated by Copaxone across the three studies. This criterion resulted in filtering out 216 probesets. Probesets were mapped to genes using the annotation available for the U133 Plus 2.0 chip from Affymetrix. FDR adjusted p values reported for genes represent the lowest FDR adjusted p value for present probesets for that gene.

### Pathway Enrichment Analysis

Upregulated and downregulated probesets were analyzed separately for pathway enrichment, using DAVID (Huang et al, Nucleic Acids Res 2009). Pathway enrichment results were visualized using volcano plots, plotting −log p values versus enrichment scores. For comparisons between Copaxone and Polimunol, upregulated or downregulated probesets with FDR-adjusted p values<0.05 and fold changes (FC) with absolute value greater than 1.1 were used for pathway enrichment. For comparisons between Copaxone® and mannitol, upregulated or downregulated probesets with FDR=adjusted p values<0.05 and FC with absolute value greater than 1.3 were used for pathway enrichment. DAVID runs were conducted December 5 and 10, 2014.

### Protein Concentrations

THP-1 cells were activated with GA or Probioglat as described above. The supernatant (1.0 mL of cell culture media) was collected at the 24 hour timepoint (to account for the time duration required for translation relative to the 6 hour mRNA data reported herein).

A Luminex assay was utilized to measure the concentrations of a panel of 45 chemokines and cytokines (in pg/ml) using Bio-Plex Human Chemokine (Bio Rad kit) and Luminex Performance Assay (R&D kit). Three of the genes that were found to significantly differ between GA and Probioglat by qRT-PCR (CXCL10, MMP9, and CCL5/RANTES) had corresponding proteins present in the Luminex panel. In addition, two other genes that were found to differ significantly between GA and Probioglat using the genome-wide microarray mRNA data were also present in the Luminex panel (CCL2, IL10).

To calculate the fold change between the protein expression levels with Probioglat and with GA, the values for the four GA batches were averaged together and compared to the value for Probioglat (Probioglat expression level/average GA expression level).

### Primary Human Monocyte Study

50 mL of blood was obtained from a healthy donor, and CD14+ cells were separated from whole blood using magnetic beads (Miltenyi Biotech). Purity was determined by FACS analysis using the following antibodies: CD14, CD15, CD16, CD45 (BD Biosciences). Into each plate of the 6 well plate 0.5 mL containing 1.0×10⁶ cells was added. In addition 0.5 mL of either Copaxone, Probioglat (100 μg/mL) or mannitol (200 μg/mL) was added, total volume in each well was 1 mL. Final concentration of Copaxone and Probioglat was 50 μg/mL and mannitol final concentration was 100 μg/mL. Cells were incubated for 6 hours at 37° C. at 5% CO₂ followed by centrifugation to pellet the cells. The cell pellets were then processed using a Qiagen RNeasy RNA purification protocol.

Expression levels of nine genes were measured using RT-PCR with GAPDH as reference transcript. Analyses reported compared Probioglat samples to GA samples as calibrator. Similar results were obtained when using mannitol as reference (i.e., the same set of genes were significantly upregulated in Probioglat relative to Copaxone). Differences in expression levels were evaluated for significance using one-sided t-tests with unequal variance.

### Example 1

### Analysis of Time-sensitive Modulation of Genes by Copaxone® Treatment in TRP-1 Monocyte Cell Line

Three time points were tested to identify the time point reflecting the greatest impact of treatment in each model system. Examining the number of genes modulated by Copaxone® relative to the mannitol control, the 6 hour time point demonstrated the largest number of genes significantly modulated by Copaxone® (6,890 with an FDR-adjusted p<0.05), over 2-fold more than at 12 hours (3,118 with FDR-adjusted p<0.05) and nearly 4-fold more than at 24 hours (1,791 with FDR-adjusted p<0.05) (Table 1). Subsequent analyses focused on the 6 hour time point, as the time point reflected the greatest impact of treatment in this model system.

TABLE 1
Numbers of genes significantly modulated by GA treatment
in THP-1 monocyte cell line at each timepoint

| | 6 hr | | 12 hr | | 24 hr | |
|---|---|---|---|---|---|---|
| | Up- | Down- | Up- | Down- | Up- | Down- |
| | regu- | regu- | regu- | regu- | regu- | regu- |
| | lated | lated | lated | lated | lated | lated |
| nominal | 3511 | 4909 | 2377 | 3430 | 1410 | 3724 |
| p < 0.05 | | | | | | |
| FDR | 2824 | 4066 | 1308 | 1810 | 606 | 1185 |
| p < 0.05 | | | | | | |
| FDR | 257 | 119 | 68 | 10 | 15 | 0 |
| p < 1e−5, | | | | | | |
| \|FC\| >= 1.5 | | | | | | |
| FDR | 557 | 508 | 210 | 50 | 57 | 6 |
| p < 1e−5, | | | | | | |
| \|FC\| >= 1.3 | | | | | | |

* FC—Fold Change; FDR—False Discovery Rate correction

### GA Mechanism of Action

To gain insight into GA's MOA, the effect of GA treatment on THP-1 human monocytes was examined, since as discussed above, antigen-presenting cells in general, including monocytes in particular, have been shown to be involved in GA treatment effects (Weber et al., Nat. Med. 2007; Kim et al, J. Immunol. Batim. Md. 2004; Burger et al., Proc. Natl. Acad. Sci. 2009; Smyth, Stat. Appl. Genet. Mol. Biol. 2004; Comi et al., Neurol. Barc. Spain 2002). In particular, the THP-1 cell line has been used to examine GA effects (Rizvi et al., Int. J. Nanomedicine, 2006). In addition, genes associated with monocytes in particular have previously been shown to be differentially expressed following treatment with a purported generic marketed by Natco in India, as compared with Copaxone (Huang et al., Nucleic Acids Res. 2009).

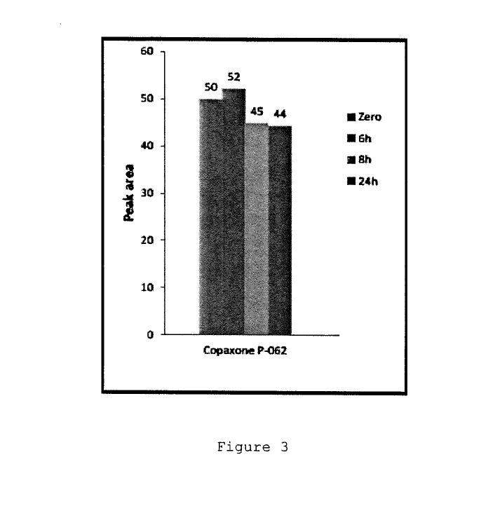

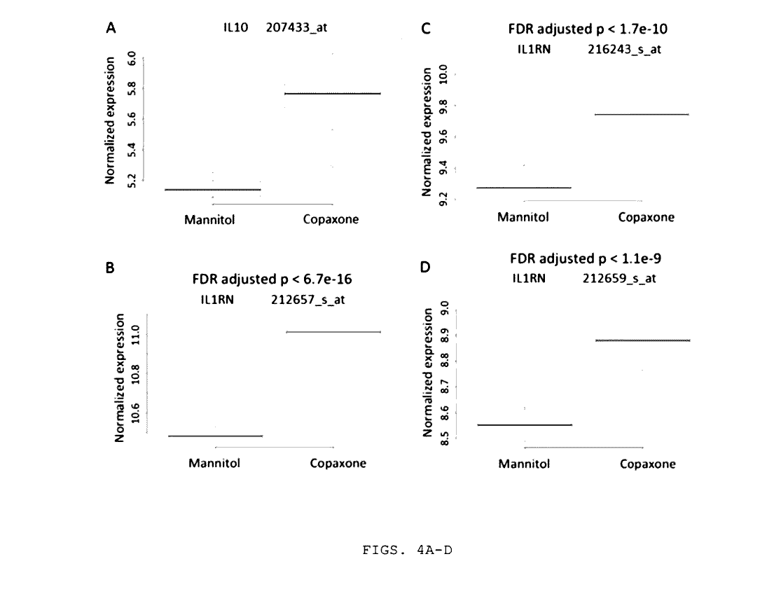

mRNA expression levels were compared between GA and control (mannitol) tested with 6 sample replicates for each of 4 batches of GA and for mannitol, using LIMMA (Sim et al., Nat. Biotechnol. 2011) (Methods). Many genes were modulated significantly (FDR-adjusted p-value<0.05) at each timepoint by treatment with branded GA (Table 1; Table 2 lists top modulated probesets). For example, at 6 hours of GA treatment, 2824 genes were significantly increased in expression (here termed upregulated) by FDR-adjusted p-value<0.05 (3511 genes by nominal p-value<0.05) and 4066 genes were significantly decreased in expression (here termed downregulated) by FDR-adjusted p-value<0.05 (4909 genes by nominal p-value<0.05). Fewer genes were significantly modulated as treatment time increased, with approximately half as many modulated at 12 hours, and approximately one-quarter at 24 hours (Table 1). We chose 6h for initial downstream analysis since this timepoint reflects the greatest impact of treatment. Levels of GA persisted in the cell culture medium in the range of 44-52 μg/mL over 24 hours (FIG. 3), indicates that this observation is unlikely to reflect a decrease in concentration of drug. Rather, this decrease in number of genes affected from early to later timepoints represents an attribute of the response of the cells to the drug.

GA impacts expression most pronouncedly at 6h (Table 2). The use of this early timepoint may also be biologically relevant given that GA is thought to be rapidly degraded at the injection site, eventually without measurable blood levels (Vieira et al., J. Immunol. Baltim. Md 1950, 2003; Thamilarasan et al., J. Neuroinflammation 2013; Comi et al, Neurol 2002, Rizvi et al, Int J Nanomed 2006).

The differentially expressed genes included several anti-inflammatory genes. For instance, IL10, the gene encoding the anti-inflammatory cytokine IL-10, was increased in expression (upregulated) at the 6 hour timepoint (FDR adjusted p value 3.1e-9; fold change (FC) 1.52; FIG. 4a). Expression of IL1RN, encoding IL-1ra, a protein that inhibits the activities of the pro-inflammatory cytokines IL-1a and IL-1b, was increased (upregulated) at all three timepoints. FIG. 4b-d shows the 6h timepoint for all present probesets (FDR adjusted p values 6.7e-16, 1.7e-10, and 1.2e-9, and FC 1.43, 1.35, and 1.26, respectively).

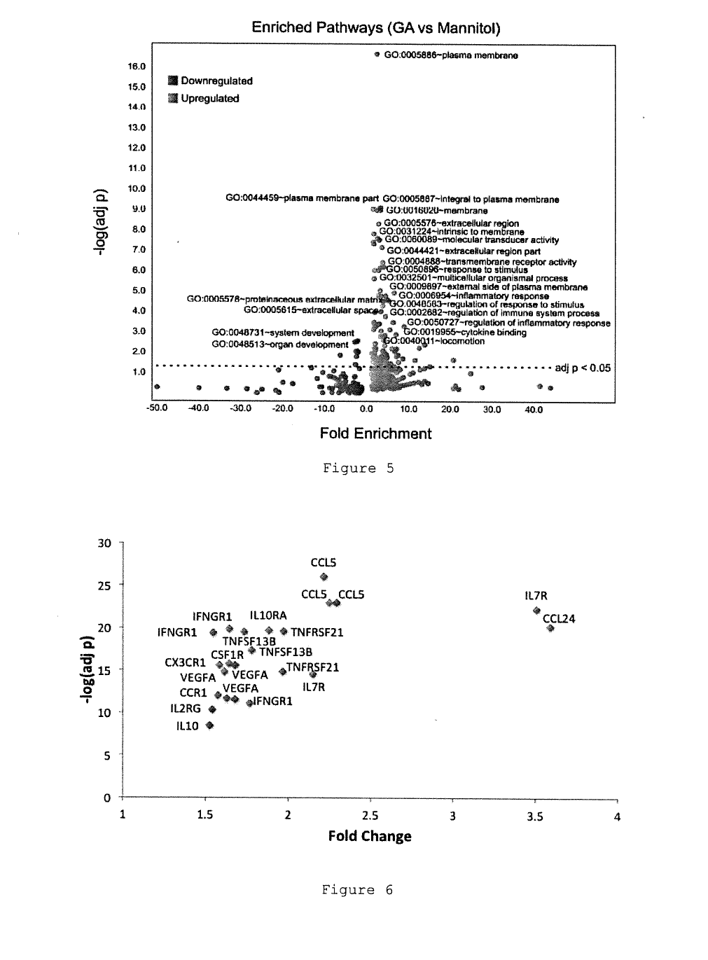

To determine whether the differentially-expressed genes related to one another in a coordinated fashion, top significantly up- and down-regulated genes were examined for pathway enrichment using DAVID as described in Methods (FIG. 5; Table 5). The top genes significantly upregulated by Copaxone in the human THP-1 cell line at 6 hours of treatment were enriched significantly (by Benjamini corrected p value<0.05) for 114 pathways (Table 5), including many immune-related pathways (FIG. 5). For example, the regulation of immune system process (GO:0002682) and cytokine-cytokine receptor interaction (hsa04060) pathways were both significantly enriched among the top upregulated genes. The top upregulated genes identified as members of the cytokine-cytokine receptor interaction pathway (hsa04060) are shown in FIG. 6. In addition, 9 pathways were significantly enriched among genes downregulated by GA (Table 5).

TABLE 2
Human monocyte study: probesets significantly modulated by
Copaxone ® relative to mannitol at 6 hours, subject to
fold-change and adjusted p value filters of 1.5 and 1e−5

| | | | | | |
|---|---|---|---|---|---|
| ID | Gene | raw.FC | AveExpr | P.Value | adj.P.Val |

Upregulated:

| | | | | | |
|---|---|---|---|---|---|
| 201005_at | CD9 | 3.77232321 | 10.8126963 | 1.14E−34 | 6.23E−30 |
| 206835_at | STATH | 7.52573433 | 10.4825707 | 1.00E−33 | 2.74E−29 |
| 1553982_a_at | RAB7B | 2.26468475 | 11.7452289 | 1.26E−31 | 2.30E−27 |
| 242871_at | PAQR5 | 3.9374814 | 9.90015385 | 7.51E−31 | 1.01E−26 |
| 1555759_a_at | CCL5 | 2.21501539 | 13.5769189 | 9.21E−31 | 1.01E−26 |
| 228766_at | — | 2.3291844 | 10.5559717 | 3.35E−30 | 3.06E−26 |
| 209555_s_at | CD36 | 2.44281389 | 9.32514677 | 7.64E−29 | 5.66E−25 |
| 203939_at | NT5E | 5.3338766 | 8.32269063 | 8.29E−29 | 5.66E−25 |
| 208789_at | PTRF | 2.82016222 | 9.15275301 | 1.88E−28 | 1.14E−24 |
| 230266_at | RAB7B | 2.23824891 | 11.7520076 | 2.34E−28 | 1.28E−24 |
| 206488_s_at | CD36 | 2.15872949 | 10.8868374 | 3.67E−28 | 1.83E−24 |
| 208121_s_at | PTPRO | 2.14699192 | 9.81795145 | 4.30E−28 | 1.96E−24 |
| 203037_s_at | MTSS1 | 3.1858517 | 11.4020576 | 7.89E−28 | 3.32E−24 |
| 1405_i_at | CCL5 | 2.29122708 | 13.3028093 | 2.79E−27 | 1.06E−23 |
| 204655_at | CCL5 | 2.25036905 | 13.1626051 | 2.90E−27 | 1.06E−23 |
| 206171_at | ADORA3 | 2.10036549 | 10.0753041 | 3.42E−27 | 1.17E−23 |
| 219386_s_at | SLAMF8 | 2.98872777 | 10.0302719 | 3.80E−27 | 1.22E−23 |
| 226218_at | — | 3.50148157 | 7.21345525 | 1.83E−26 | 5.55E−23 |
| 218559_s_at | MAFB | 2.64025573 | 12.4234341 | 3.40E−26 | 9.78E−23 |
| 223125_s_at | C1ORF21 | 2.21866076 | 11.0023707 | 4.15E−26 | 1.13E−22 |
| 203887_s_at | THBD | 1.79337816 | 12.8191102 | 4.45E−26 | 1.15E−22 |
| 227265_at | KIAA1505 | 2.50321069 | 9.85031558 | 4.61E−26 | 1.15E−22 |
| 205495_s_at | GNLY | 2.40116081 | 7.73128126 | 5.34E−26 | 1.27E−22 |
| 216250_s_at | LPXN | 2.1444933 | 11.2374219 | 1.42E−25 | 3.23E−22 |
| 237252_at | THBD | 2.06547107 | 9.51011605 | 2.22E−25 | 4.86E−22 |
| 235457_at | MAML2 | 2.62488399 | 8.91497905 | 5.79E−25 | 1.22E−21 |
| 225171_at | ARHGAP18 | 1.83225951 | 10.7080346 | 6.66E−25 | 1.35E−21 |
| 204684_at | NPTX1 | 2.87807656 | 10.9346668 | 1.40E−24 | 2.74E−21 |
| 230925_at | APBB1IP | 1.94283823 | 12.7655513 | 2.02E−24 | 3.81E−21 |
| 201590_x_at | ANXA2 | 1.68959054 | 13.1436175 | 3.46E−24 | 6.31E−21 |
| 206157_at | PTX3 | 1.98670615 | 11.2102165 | 3.62E−24 | 6.38E−21 |
| 216080_s_at | FADS3 | 2.27239911 | 9.71739825 | 3.75E−24 | 6.39E−21 |
| 221463_at | CCL24 | 3.58807723 | 8.40481021 | 3.93E−24 | 6.39E−21 |
| 210427_x_at | ANXA2 | 1.67415609 | 13.1118777 | 3.98E−24 | 6.39E−21 |
| 213503_x_at | ANXA2 | 1.72399354 | 13.1135447 | 4.18E−24 | 6.51E−21 |
| 238681_at | GDPD1 | 2.92185146 | 7.19901556 | 4.29E−24 | 6.51E−21 |
| 202458_at | PRSS23 | 2.98100135 | 7.79000143 | 5.03E−24 | 7.43E−21 |
| 203761_at | SLA | 1.78030673 | 11.2608124 | 6.52E−24 | 9.38E−21 |
| 1558397_at | — | 2.20691751 | 9.17869031 | 8.35E−24 | 1.17E−20 |
| 214054_at | DOK2 | 1.67191836 | 12.2431311 | 8.80E−24 | 1.20E−20 |
| 228937_at | C13ORF31 | 2.42816683 | 9.90878354 | 9.66E−24 | 1.29E−20 |
| 211676_s_at | IFNGR1 | 1.64276331 | 12.0995889 | 1.08E−23 | 1.41E−20 |
| 230966_at | IL4I1 | 3.68319718 | 10.6065738 | 1.18E−23 | 1.50E−20 |
| 208816_x_at | ANXA2P2 | 1.61606927 | 11.6007732 | 1.33E−23 | 1.66E−20 |
| 204912_at | IL10RA | 1.88097974 | 10.4596073 | 1.53E−23 | 1.83E−20 |
| 220014_at | LOC51334 | 3.95258058 | 6.49298277 | 1.54E−23 | 1.83E−20 |
| 218856_at | TNFRSF21 | 1.97277863 | 11.9973199 | 2.06E−23 | 2.40E−20 |
| 34210_at | CD52 | 1.74841624 | 11.3194919 | 2.57E−23 | 2.92E−20 |
| 211564_s_at | PDLIM4 | 2.13518707 | 9.68326105 | 2.72E−23 | 3.03E−20 |
| 223502_s_at | TNFSF13B | 1.73530598 | 10.5512407 | 2.94E−23 | 3.21E−20 |
| 202727_s_at | IFNGR1 | 1.5463282 | 12.4669611 | 3.25E−23 | 3.48E−20 |
| 201243_s_at | ATP1B1 | 2.26738679 | 11.039008 | 4.33E−23 | 4.56E−20 |
| 209606_at | PSCDBP | 2.53767762 | 8.05776375 | 4.93E−23 | 5.08E−20 |
| 222670_s_at | MAFB | 2.65652588 | 11.7521547 | 6.27E−23 | 6.35E−20 |
| 223394_at | SERTAD1 | 1.65031524 | 9.76915308 | 6.91E−23 | 6.87E−20 |
| 214175_x_at | PDLIM4 | 1.85137891 | 9.80931564 | 8.66E−23 | 8.45E−20 |
| 223660_at | ADORA3 | 1.89720977 | 9.3830598 | 9.01E−23 | 8.64E−20 |
| 203217_s_at | ST3GAL5 | 1.78903169 | 9.10901818 | 1.17E−22 | 1.11E−19 |
| 204257_at | FADS3 | 1.78672893 | 9.74459459 | 1.23E−22 | 1.14E−19 |
| 223398_at | C9ORF89 | 1.54418891 | 11.0771528 | 1.25E−22 | 1.14E−19 |
| 211986_at | AHNAK | 1.64967948 | 12.0209047 | 1.63E−22 | 1.45E−19 |
| 201278_at | DAB2 | 1.56823308 | 9.36397364 | 1.64E−22 | 1.45E−19 |
| 229797_at | MCOLN3 | 2.09980647 | 10.05118 | 2.01E−22 | 1.75E−19 |
| 202284_s_at | CDKN1A | 1.789083 | 11.1500265 | 2.69E−22 | 2.22E−19 |
| 228579_at | — | 1.73158064 | 8.85026747 | 3.40E−22 | 2.78E−19 |
| 204653_at | TFAP2A | 1.98518712 | 8.32878609 | 5.70E−22 | 4.39E−19 |
| 201952_at | ALCAM | 1.52577304 | 12.9059918 | 6.14E−22 | 4.66E−19 |
| 202087_s_at | CTSL | 1.91284953 | 11.1924209 | 6.47E−22 | 4.84E−19 |
| 208978_at | CRIP2 | 1.99640335 | 10.1060568 | 6.82E−22 | 5.04E−19 |
| 219434_at | TREM1 | 1.77683606 | 11.1148543 | 6.95E−22 | 5.07E−19 |
| 235911_at | MFI2 | 1.81455526 | 10.1578351 | 7.75E−22 | 5.57E−19 |
| 225173_at | ARHGAP18 | 1.85956949 | 10.0159515 | 7.95E−22 | 5.65E−19 |
| 204661_at | CD52 | 1.6752998 | 11.8608631 | 9.08E−22 | 6.28E−19 |
| 202756_s_at | GPC1 | 1.88792383 | 8.93231998 | 9.08E−22 | 6.28E−19 |
| 227240_at | NGEF | 1.97393626 | 9.35225393 | 9.96E−22 | 6.66E−19 |
| 220484_at | MCOLN3 | 2.10889009 | 9.93061659 | 9.98E−22 | 6.66E−19 |
| 214297_at | CSPG4 | 1.98668454 | 8.74794884 | 9.99E−22 | 6.66E−19 |
| 203355_s_at | PSD3 | 1.68707305 | 9.97354098 | 1.13E−21 | 7.47E−19 |
| 212386_at | — | 1.80977269 | 8.4443852 | 1.22E−21 | 7.91E−19 |
| 201242_s_at | ATP1B1 | 2.36020561 | 10.833866 | 1.62E−21 | 1.03E−18 |
| 203760_s_at | SLA | 1.76467647 | 10.2984296 | 1.71E−21 | 1.07E−18 |
| 204475_at | MMP1 | 4.89045646 | 6.14908101 | 1.97E−21 | 1.21E−18 |
| 228964_at | PRDM1 | 2.42316104 | 9.16116313 | 2.05E−21 | 1.24E−18 |
| 205566_at | ABHD2 | 1.61609464 | 9.56280738 | 2.12E−21 | 1.27E−18 |
| 224990_at | LOC201895 | 1.75486717 | 9.56572435 | 2.15E−21 | 1.28E−18 |
| 214830_at | SLC38A6 | 1.70065597 | 9.60777602 | 2.38E−21 | 1.38E−18 |
| 222876_s_at | CENTA2 | 1.62337809 | 9.52111995 | 3.45E−21 | 1.92E−18 |
| 218501_at | ARHGEF3 | 1.72054917 | 9.02047119 | 3.66E−21 | 2.02E−18 |
| 206134_at | ADAMDEC1 | 1.90734854 | 8.46538539 | 3.83E−21 | 2.10E−18 |
| 219385_at | SLAMF8 | 2.3601546 | 8.87175449 | 4.64E−21 | 2.49E−18 |
| 212298_at | NRP1 | 2.26706147 | 9.0154209 | 5.27E−21 | 2.77E−18 |
| 1556314_a_at | — | 1.94563197 | 9.96244884 | 5.97E−21 | 3.05E−18 |
| 204834_at | FGL2 | 2.41220628 | 9.04212862 | 7.43E−21 | 3.63E−18 |
| 227052_at | — | 1.7292497 | 9.65138189 | 8.49E−21 | 4.11E−18 |
| 223501_at | — | 1.77755998 | 10.4775991 | 9.58E−21 | 4.59E−18 |
| 238669_at | PTGS1 | 1.79526928 | 9.00713066 | 1.52E−20 | 7.11E−18 |
| 203936_s_at | MMP9 | 1.83742157 | 9.54268719 | 2.01E−20 | 9.33E−18 |
| 205128_x_at | PTGS1 | 1.50682893 | 10.8982825 | 2.46E−20 | 1.12E−17 |
| 223019_at | C9ORF88 | 1.71440506 | 9.42451447 | 2.48E−20 | 1.12E−17 |
| 218223_s_at | PLEKHO1 | 1.76062083 | 9.13759715 | 2.55E−20 | 1.14E−17 |
| 226545_at | CD109 | 2.22747108 | 10.1210564 | 2.62E−20 | 1.17E−17 |
| 37145_at | GNLY | 2.52474166 | 7.62618078 | 2.77E−20 | 1.21E−17 |
| 211661_x_at | PTAFR | 1.65459655 | 9.40348625 | 3.20E−20 | 1.39E−17 |
| 223434_at | GBP3 | 1.79281477 | 8.94069901 | 5.65E−20 | 2.39E−17 |
| 221011_s_at | LBH /// LOC653743 | 2.14546482 | 7.91354368 | 6.67E−20 | 2.79E−17 |
| 227478_at | LOC284262 | 2.55523487 | 8.16720299 | 7.57E−20 | 3.09E−17 |
| 205933_at | SETBP1 | 2.06852059 | 8.31470678 | 9.65E−20 | 3.88E−17 |
| 223126_s_at | C1ORF21 | 2.0098249 | 9.34926366 | 1.00E−19 | 3.99E−17 |
| 214255_at | ATP10A | 1.87404352 | 8.48741153 | 1.17E−19 | 4.64E−17 |
| 203888_at | THBD | 1.74251135 | 12.1003768 | 1.32E−19 | 5.18E−17 |
| 239761_at | GCNT1 | 1.60737016 | 9.89061934 | 1.78E−19 | 6.90E−17 |
| 205505_at | GCNT1 | 1.68341734 | 9.51667582 | 1.80E−19 | 6.93E−17 |
| 203665_at | HMOX1 | 2.77855009 | 12.0276078 | 1.83E−19 | 7.00E−17 |
| 228490_at | ABHD2 | 1.7612845 | 9.44137483 | 1.92E−19 | 7.27E−17 |
| 213428_s_at | COL6A1 | 1.6365876 | 9.11385826 | 2.21E−19 | 8.27E−17 |
| 202748_at | GBP2 | 2.05214857 | 8.5030424 | 2.71E−19 | 1.01E−16 |
| 63825_at | ABHD2 | 1.60237791 | 9.12592031 | 3.53E−19 | 1.28E−16 |
| 205542_at | STEAP1 | 1.7513289 | 7.91561507 | 4.15E−19 | 1.49E−16 |
| 215813_s_at | PTGS1 | 1.51668643 | 10.9189252 | 4.31E−19 | 1.53E−16 |
| 205891_at | ADORA2B | 1.56662939 | 10.2505629 | 4.70E−19 | 1.66E−16 |
| 218613_at | LOC653754 | 1.55398123 | 9.29556045 | 4.95E−19 | 1.73E−16 |
| 205076_s_at | MTMR11 | 1.59326395 | 11.2341764 | 5.05E−19 | 1.76E−16 |
| 212086_at | LMNA | 1.50357006 | 12.1481348 | 5.14E−19 | 1.78E−16 |
| 203104_at | CSF1R | 1.64383545 | 11.9446038 | 5.33E−19 | 1.83E−16 |
| 228368_at | ARHGAP20 | 2.23117651 | 8.70947261 | 5.37E−19 | 1.84E−16 |
| 227889_at | AYTL1 | 1.59282395 | 10.8879284 | 5.43E−19 | 1.84E−16 |
| 203797_at | VSNL1 | 1.7623632 | 9.20419419 | 5.45E−19 | 1.84E−16 |
| 210145_at | PLA2G4A | 1.69920287 | 9.86166848 | 5.50E−19 | 1.84E−16 |
| 220307_at | CD244 | 1.51030196 | 11.2887381 | 6.64E−19 | 2.20E−16 |
| 201280_s_at | DAB2 | 1.6207775 | 8.250618 | 7.03E−19 | 2.30E−16 |
| 205898_at | CX3CR1 | 1.5834422 | 12.3392251 | 7.53E−19 | 2.45E−16 |
| 212171_x_at | VEGF | 1.67886192 | 11.082533 | 8.08E−19 | 2.61E−16 |
| 205419_at | EBI2 | 1.87963971 | 10.3117587 | 8.24E−19 | 2.64E−16 |
| 242794_at | MAML3 | 1.71070972 | 8.2000023 | 8.71E−19 | 2.76E−16 |
| 1553141_at | C13ORF31 | 2.23957531 | 8.21534882 | 9.28E−19 | 2.90E−16 |
| 1556034_s_at | MTMR11 | 1.54656669 | 10.734173 | 9.32E−19 | 2.90E−16 |
| 202436_s_at | CYP1B1 | 2.02651623 | 11.7446834 | 1.35E−18 | 4.08E−16 |
| 205718_at | ITGB7 | 1.61859456 | 10.0388412 | 1.64E−18 | 4.92E−16 |
| 222062_at | IL27RA | 1.84145262 | 8.80213383 | 1.77E−18 | 5.26E−16 |
| 1565752_at | FGD2 | 2.03973925 | 8.7032554 | 1.80E−18 | 5.32E−16 |
| 203140_at | BCL6 | 1.6321005 | 9.84693758 | 1.88E−18 | 5.54E−16 |
| 209568_s_at | RGL1 | 1.79068455 | 9.03375054 | 2.04E−18 | 5.92E−16 |
| 212977_at | CXCR7 | 1.95857569 | 9.06724806 | 2.73E−18 | 7.74E−16 |
| 225809_at | DKEZP564O0823 | 2.61498482 | 6.81686881 | 3.21E−18 | 8.90E−16 |
| 223723_at | MFI2 | 1.83918768 | 7.73004392 | 3.45E−18 | 9.49E−16 |
| 204141_at | TUBB2A | 1.92337874 | 7.9645182 | 3.55E−18 | 9.72E−16 |
| 219358_s_at | CENTA2 | 1.61743685 | 10.3689956 | 3.67E−18 | 9.97E−16 |
| 218280_x_at | HIST2H2AA /// LOC653610 ///H2A/R | 1.92645343 | 11.1017962 | 3.91E−18 | 1.05E−15 |
| 200884_at | CKB | 1.62119168 | 10.3585418 | 3.91E−18 | 1.05E−15 |
| 213265_at | PGA5 /// LOC643834 /// LOC643847 | 1.54256466 | 9.03735387 | 4.18E−18 | 1.12E−15 |
| 228499_at | PFKFB4 | 1.54265427 | 8.78361163 | 4.58E−18 | 1.22E−15 |
| 214581_x_at | TNFRSF21 | 1.96726151 | 10.3862452 | 5.13E−18 | 1.35E−15 |
| 219256_s_at | SH3TC1 | 1.81211933 | 8.14371592 | 5.59E−18 | 1.46E−15 |
| 227134_at | SYTL1 | 1.63431503 | 10.2085878 | 5.74E−18 | 1.50E−15 |
| 210512_s_at | VEGF | 1.61269337 | 12.1528461 | 5.88E−18 | 1.52E−15 |
| 220066_at | CARD15 | 1.63208119 | 8.81134661 | 6.12E−18 | 1.58E−15 |
| 207610_s_at | EMR2 | 1.96904599 | 7.45239164 | 6.31E−18 | 1.62E−15 |
| 225337_at | ABHD2 | 1.52226303 | 10.585684 | 6.47E−18 | 1.65E−15 |
| 1553906_s_at | FGD2 | 1.56817402 | 11.6738144 | 8.15E−18 | 2.05E−15 |
| 210757_x_at | DAB2 | 1.51596174 | 9.71603785 | 8.84E−18 | 2.22E−15 |
| 205306_x_at | KMO | 1.52538689 | 9.01246744 | 1.07E−17 | 2.64E−15 |
| 203980_at | FABP4 | 2.44504844 | 7.91709543 | 1.14E−17 | 2.81E−15 |
| 205798_at | IL7R | 2.15224639 | 6.90018766 | 1.20E−17 | 2.94E−15 |
| 235299_at | — | 2.92455443 | 6.38887226 | 1.23E−17 | 2.99E−15 |
| 201422_at | IFI30 | 1.53617841 | 12.1952 | 1.33E−17 | 3.23E−15 |
| 201951_at | ALCAM | 1.56151412 | 11.2835886 | 1.36E−17 | 3.29E−15 |
| 219412_at | RAB38 | 1.69022517 | 7.7984244 | 1.40E−17 | 3.36E−15 |
| 214290_s_at | HIST2H2AA /// LOC653610 /// H2A/R | 1.8793137 | 12.5116024 | 1.77E−17 | 4.19E−15 |
| 223092_at | ANKH | 1.74621066 | 9.31182429 | 1.93E−17 | 4.53E−15 |
| 1552553_a_at | CARD12 | 1.8300836 | 8.04038883 | 1.98E−17 | 4.61E−15 |
| 1565754_x_at | FGD2 | 2.05302245 | 8.76658914 | 1.98E−17 | 4.61E−15 |
| 1562475_at | DKFZP686O1327 | 1.84932663 | 8.38987825 | 2.25E−17 | 5.18E−15 |
| 202435_s_at | CYP1B1 | 1.96043252 | 11.3853309 | 2.26E−17 | 5.18E−15 |
| 222877_at | NRP2 | 2.43002847 | 6.1162318 | 2.37E−17 | 5.41E−15 |
| 201125_s_at | ITGB5 | 1.55447321 | 9.61610321 | 2.48E−17 | 5.62E−15 |
| 204222_s_at | GLIPR1 | 1.64989211 | 10.4691144 | 3.30E−17 | 7.31E−15 |
| 1553142_at | C13ORF31 | 2.72312753 | 7.05894587 | 3.42E−17 | 7.51E−15 |
| 201279_s_at | DAB2 | 1.51128632 | 9.36621678 | 3.53E−17 | 7.64E−15 |
| 203234_at | UPP1 | 1.74387737 | 9.56780561 | 3.97E−17 | 8.52E−15 |
| 226136_at | — | 1.5023264 | 9.021618 | 4.37E−17 | 9.27E−15 |
| 206206_at | CD180 | 1.61188665 | 9.82309399 | 6.95E−17 | 1.43E−14 |
| 212387_at | — | 1.62401877 | 7.80816226 | 1.05E−16 | 2.07E−14 |
| 204268_at | S100A2 | 1.79310512 | 8.64238178 | 1.22E−16 | 2.38E−14 |
| 209122_at | ADFP | 1.61478632 | 13.4092966 | 1.23E−16 | 2.41E−14 |
| 219994_at | APBB1IP | 1.87740546 | 9.6208094 | 1.42E−16 | 2.74E−14 |
| 210095_s_at | IGFBP3 | 2.98798358 | 8.63072598 | 1.43E−16 | 2.74E−14 |
| 227948_at | FGD4 | 1.62553487 | 7.73111329 | 1.54E−16 | 2.92E−14 |
| 243483_at | TRPM8 | 2.13837999 | 7.16652124 | 1.62E−16 | 3.04E−14 |
| 212062_at | ATP9A | 2.12888023 | 7.50745486 | 1.77E−16 | 3.29E−14 |
| 218502_s_at | TRPS1 | 1.61387671 | 8.89822927 | 1.95E−16 | 3.60E−14 |
| 213891_s_at | — | 1.67207935 | 8.30917084 | 2.03E−16 | 3.68E−14 |
| 200878_at | EPAS1 | 1.51714595 | 9.20362299 | 2.04E−16 | 3.69E−14 |
| 201565_s_at | ID2 | 1.52829092 | 12.7810873 | 2.26E−16 | 4.06E−14 |
| 212190_at | SERPINE2 | 1.92452066 | 9.90116936 | 2.57E−16 | 4.60E−14 |
| 224480_s_at | LPAAT-THETA | 1.96245654 | 8.74709429 | 2.64E−16 | 4.67E−14 |
| 220333_at | PAQR5 | 3.58344803 | 7.84174393 | 2.70E−16 | 4.76E−14 |
| 218589_at | P2RY5 | 1.65149795 | 9.3651465 | 3.00E−16 | 5.23E−14 |
| 225166_at | ARHGAP18 | 1.75622288 | 7.33398136 | 3.29E−16 | 5.64E−14 |
| 226066_at | MITF | 1.750697 | 7.87439597 | 3.35E−16 | 5.73E−14 |
| 221211_s_at | C21ORF7 | 2.08949444 | 5.978895 | 3.41E−16 | 5.80E−14 |
| 225842_at | PHLDA1 | 2.51724542 | 8.55236084 | 3.48E−16 | 5.89E−14 |
| 1554992_at | RASGRF1 | 1.56735654 | 6.94135257 | 3.77E−16 | 6.35E−14 |
| 225097_at | HIPK2 | 1.50753986 | 10.7130384 | 3.84E−16 | 6.45E−14 |
| 212464_s_at | FN1 | 1.70809888 | 9.26484812 | 4.53E−16 | 7.53E−14 |
| 240076_at | — | 1.5987436 | 8.40204375 | 4.66E−16 | 7.73E−14 |
| 203798_s_at | VSNL1 | 1.91644457 | 7.87623269 | 4.84E−16 | 7.92E−14 |
| 204575_s_at | MMP19 /// LOC652543 | 1.83367508 | 7.70439226 | 4.88E−16 | 7.96E−14 |
| 204465_s_at | INA | 1.60147629 | 8.20221976 | 5.17E−16 | 8.36E−14 |
| 201566_x_at | ID2 /// ID28 | 1.57371123 | 10.7833858 | 5.52E−16 | 8.87E−14 |
| 230360_at | GLDN | 1.98086959 | 6.74193069 | 6.80E−16 | 1.07E−13 |
| 222146_s_at | TCF4 | 1.54556332 | 7.51472351 | 8.25E−16 | 1.28E−13 |
| 224341_x_at | TLR4 | 1.7396424 | 8.97197657 | 8.61E−16 | 1.32E−13 |
| 219637_at | ARMC9 | 1.80286893 | 7.06720581 | 8.87E−16 | 1.35E−13 |
| 211138_s_at | KMO | 1.54711526 | 8.79132849 | 9.12E−16 | 1.38E−13 |
| 202437_s_at | CYP1B1 | 1.80921395 | 12.1893518 | 9.54E−16 | 1.43E−13 |
| 235458_at | HAVCR2 | 2.48536941 | 7.7122454 | 9.75E−16 | 1.46E−13 |
| 203922_s_at | CYBB | 1.58449815 | 10.3814577 | 1.07E−15 | 1.58E−13 |
| 212143_s_at | IGFBP3 | 2.76854721 | 7.92968375 | 1.26E−15 | 1.82E−13 |
| 227716_at | UBXD5 | 1.51942114 | 9.002862 | 1.28E−15 | 1.83E−13 |
| 214211_at | FTH1 | 1.55999567 | 12.4970466 | 1.33E−15 | 1.90E−13 |
| 229004_at | — | 1.51039984 | 9.12368109 | 1.55E−15 | 2.18E−13 |
| 202609_at | EPS8 | 1.5924097 | 8.52061192 | 1.82E−15 | 2.52E−13 |
| 217757_at | A2M | 1.67532065 | 8.4520092 | 1.98E−15 | 2.73E−13 |
| 1552798_a_at | TLR4 | 1.62695353 | 9.04302518 | 2.09E−15 | 2.88E−13 |
| 57715_at | FAM26B | 1.79873285 | 7.75582627 | 2.40E−15 | 3.27E−13 |
| 226282_at | — | 1.60485814 | 9.41013847 | 2.84E−15 | 3.80E−13 |
| 222838_at | SLAMF7 | 1.86121844 | 7.05728625 | 3.11E−15 | 4.13E−13 |
| 216442_x_at | FN1 | 1.69780521 | 10.1409361 | 3.73E−15 | 4.87E−13 |
| 201324_at | EMP1 | 2.82050293 | 6.11305975 | 3.81E−15 | 4.94E−13 |
| 210264_at | GPR35 | 1.59109619 | 9.60072153 | 3.91E−15 | 5.05E−13 |
| 222858_s_at | DAPP1 | 1.6313133 | 10.6750257 | 4.54E−15 | 5.78E−13 |
| 209684_at | RIN2 | 2.16288563 | 7.25953396 | 5.48E−15 | 6.84E−13 |
| 201069_at | MMP2 | 1.51386957 | 8.69855532 | 6.09E−15 | 7.55E−13 |
| 204998_s_at | ATF5 | 1.63500149 | 10.6267979 | 6.14E−15 | 7.60E−13 |
| 202434_s_at | CYP1B1 | 2.2027314 | 9.83119314 | 6.94E−15 | 8.52E−13 |
| 205099_s_at | CCR1 | 1.57264838 | 9.76018397 | 7.25E−15 | 8.85E−13 |
| 207233_s_at | MITF | 1.66865012 | 8.30250431 | 8.03E−15 | 9.72E−13 |
| 205552_s_at | OAS1 | 1.79452532 | 7.29881386 | 8.53E−15 | 1.02E−12 |
| 226550_at | — | 1.79237731 | 7.77105086 | 9.20E−15 | 1.09E−12 |
| 212090_s_at | MTUS1 | 1.62315248 | 8.50418445 | 9.61E−15 | 1.13E−12 |
| 222651_s_at | TRPS1 | 1.5940306 | 10.0878238 | 9.64E−15 | 1.13E−12 |
| 211719_x_at | FN1 | 1.74293625 | 10.1007312 | 9.80E−15 | 1.15E−12 |
| 227609_at | EPSTI1 | 1.73531372 | 7.62913716 | 1.06E−14 | 1.24E−12 |
| 1558105_a_at | — | 1.89855059 | 6.81046182 | 1.07E−14 | 1.25E−12 |
| 225207_at | PDK4 | 2.24881528 | 8.76581849 | 1.08E−14 | 1.25E−12 |
| 237160_at | CCDC83 | 2.26816884 | 5.8347316 | 1.15E−14 | 1.33E−12 |
| 211026_s_at | MGLL | 1.80987658 | 8.99536888 | 1.33E−14 | 1.53E−12 |
| 229450_at | — | 2.07979683 | 6.90012377 | 1.42E−14 | 1.62E−12 |
| 215602_at | FGD2 | 2.01984347 | 7.85983935 | 1.54E−14 | 1.74E−12 |
| 228708_at | RAB27B | 1.75996236 | 8.57073526 | 1.63E−14 | 1.83E−12 |
| 210258_at | RGS13 | 2.4181722 | 5.06675503 | 1.77E−14 | 1.97E−12 |
| 210895_s_at | CD86 | 1.74036933 | 7.80363779 | 1.88E−14 | 2.07E−12 |
| 223672_at | SGIP1 | 1.58033388 | 8.69396513 | 1.96E−14 | 2.14E−12 |
| 210513_s_at | VEGF | 1.62960752 | 9.66705476 | 2.08E−14 | 2.25E−12 |
| 212614_at | ARID5B | 1.64330835 | 6.34592171 | 2.09E−14 | 2.26E−12 |
| 1553995_a_at | NT5E | 2.06345466 | 6.49987213 | 2.17E−14 | 2.32E−12 |
| 233540_s_at | CDK5RAP2 | 1.77928193 | 10.6899414 | 2.34E−14 | 2.48E−12 |
| 204059_s_at | ME1 | 1.78078662 | 11.5066468 | 2.46E−14 | 2.58E−12 |
| 211527_x_at | VEGF | 1.68079685 | 9.90807927 | 2.48E−14 | 2.60E−12 |
| 214724_at | DIXDC1 | 1.55892061 | 7.67060007 | 2.63E−14 | 2.74E−12 |
| 227556_at | NME7 | 2.07486651 | 9.31851129 | 2.92E−14 | 3.02E−12 |
| 237442_at | APBB1IP | 1.97657406 | 7.17373032 | 3.09E−14 | 3.18E−12 |
| 224218_s_at | TRPS1 | 1.52587932 | 8.16882094 | 3.38E−14 | 3.46E−12 |
| 225188_at | RAPH1 | 1.90298066 | 6.31647018 | 3.68E−14 | 3.73E−12 |
| 244414_at | MAML2 | 2.39160043 | 8.05798482 | 3.70E−14 | 3.74E−12 |
| 209906_at | C3AR1 | 1.62034968 | 8.32122922 | 3.73E−14 | 3.76E−12 |
| 210510_s_at | NRP1 | 1.56055098 | 7.3171434 | 3.77E−14 | 3.78E−12 |
| 226629_at | SLC43A2 | 1.57641868 | 8.50211433 | 3.97E−14 | 3.94E−12 |
| 237030_at | ACPP | 1.53890938 | 8.05225501 | 4.15E−14 | 4.10E−12 |
| 213338_at | TMEM158 | 1.73773739 | 10.7585184 | 4.18E−14 | 4.13E−12 |
| 219926_at | POPDC3 | 2.12181972 | 6.63647483 | 5.99E−14 | 5.68E−12 |
| 200897_s_at | PALLD | 1.64882729 | 8.35346573 | 6.07E−14 | 5.74E−12 |
| 208436_s_at | IRF7 | 1.55075885 | 8.74418094 | 6.34E−14 | 5.96E−12 |
| 228438_at | TRPA1 | 1.65933581 | 10.3950167 | 6.81E−14 | 6.30E−12 |
| 227747_at | — | 1.55198677 | 7.93194342 | 6.84E−14 | 6.32E−12 |
| 1554867_a_at | LOC51334 | 1.86507425 | 7.40812044 | 6.90E−14 | 6.36E−12 |
| 1563445_x_at | CTSLL3 | 1.82851184 | 6.84405898 | 9.09E−14 | 8.12E−12 |
| 242903_at | IFNGR1 | 1.7678462 | 7.97757472 | 9.49E−14 | 8.42E−12 |
| 229900_at | CD109 | 1.54089288 | 8.62103824 | 1.05E−13 | 9.24E−12 |
| 210495_x_at | FN1 | 1.61505134 | 10.0973935 | 1.07E−13 | 9.43E−12 |
| 218854_at | SART2 | 1.54263807 | 8.36626559 | 1.13E−13 | 9.81E−12 |
| 224989_at | — | 1.78010467 | 7.75893731 | 1.17E−13 | 1.02E−11 |
| 238638_at | SLC37A2 | 1.63842433 | 9.93438242 | 1.31E−13 | 1.13E−11 |
| 212382_at | — | 1.60505834 | 7.06996616 | 1.35E−13 | 1.15E−11 |
| 209047_at | AQP1 | 2.44653569 | 7.84720827 | 1.38E−13 | 1.17E−11 |
| 236345_at | TBXAS1 | 1.66860916 | 8.46774141 | 1.40E−13 | 1.18E−11 |
| 228873_at | COL22A1 | 1.56093003 | 7.43196243 | 1.41E−13 | 1.19E−11 |
| 239519_at | NRP1 | 2.19110297 | 5.55763801 | 1.43E−13 | 1.20E−11 |
| 220935_s_at | CDK5RAP2 | 1.84541222 | 10.4100106 | 1.54E−13 | 1.28E−11 |
| 1560228_at | SNAI3 | 1.91948596 | 7.8146015 | 1.56E−13 | 1.30E−11 |
| 218934_s_at | HSPB7 | 1.65040526 | 8.10534113 | 2.40E−13 | 1.90E−11 |
| 210146_x_at | LILRB2 | 1.9982938 | 6.76423766 | 2.86E−13 | 2.22E−11 |
| 209348_s_at | MAF | 1.56472875 | 8.72338323 | 3.17E−13 | 2.42E−11 |
| 1558569_at | LOC645238 | 1.75050375 | 7.56283286 | 3.25E−13 | 2.47E−11 |
| 243856_at | — | 1.73115631 | 8.03640769 | 3.81E−13 | 2.86E−11 |
| 223798_at | SLC41A2 | 1.94681117 | 6.54582797 | 3.90E−13 | 2.92E−11 |
| 215990_s_at | BCL6 | 1.50671943 | 8.04953142 | 4.81E−13 | 3.52E−11 |
| 202869_at | OAS1 | 1.63942747 | 6.73520018 | 5.30E−13 | 3.86E−11 |
| 225631_at | KIAA1706 | 1.53753899 | 7.78142994 | 5.56E−13 | 4.03E−11 |
| 204116_at | IL2RG | 1.53970115 | 7.99806857 | 5.88E−13 | 4.22E−11 |
| 225189_s_at | RAPH1 | 1.85367315 | 6.14549622 | 6.27E−13 | 4.47E−11 |
| 211066_x_at | PCDHGC3 /// PCDHGB4 /// PCDHGA8 | 1.58678656 | 10.4111947 | 7.90E−13 | 5.53E−11 |
| | /// PCDHGA12 /// PCDHGC5 /// | | | | |
| | PCDHGC4 /// PCDHGB7 /// PCDHGB6 | | | | |
| | /// PCDHGB5 /// PCDHGB3 /// | | | | |
| | PCDHGB2 /// PCDHGB1 /// | | | | |
| | PCDHGA11 /// PCDHGA10 /// | | | | |
| | PCDHGA9 /// PCDHGA7 /// PCDHGA6 | | | | |
| | /// PCDHGA5 /// PCDHGA4 /// | | | | |
| | PCDHGA3 /// PCDHGA2 /// PCDHGA1 | | | | |
| 221266_s_at | TM7SF4 | 1.83671444 | 6.22762311 | 8.02E−13 | 5.60E−11 |
| 1560485_at | HIVEP1 | 1.6480621 | 6.99003908 | 8.06E−13 | 5.62E−11 |
| 204881_s_at | UGCG | 1.5102382 | 9.51481557 | 8.38E−13 | 5.83E−11 |
| 235286_at | CKLF | 1.61741462 | 8.34288205 | 9.26E−13 | 6.36E−11 |
| 205003_at | DOCK4 | 1.5112689 | 8.20388936 | 1.02E−12 | 6.92E−11 |
| 218686_s_at | RHBDF1 | 1.51723286 | 7.92482272 | 1.05E−12 | 7.08E−11 |
| 221565_s_at | FAM26B | 1.59642371 | 7.91366407 | 1.09E−12 | 7.32E−11 |
| 206675_s_at | SKIL | 1.61042514 | 7.42083013 | 1.16E−12 | 7.69E−11 |
| 242358_at | — | 2.20249712 | 6.04968315 | 1.24E−12 | 8.20E−11 |
| 241392_at | TMEM39A | 1.61384348 | 7.05009494 | 1.87E−12 | 1.19E−10 |
| 232333_at | MAML2 | 1.68481156 | 8.01950459 | 2.06E−12 | 1.30E−10 |
| 228762_at | LFNG | 1.55190457 | 9.0403074 | 2.08E−12 | 1.31E−10 |
| 242907_at | GBP2 | 2.14738965 | 6.03707956 | 2.72E−12 | 1.64E−10 |
| 203753_at | TCF4 | 1.50293289 | 7.66442858 | 2.78E−12 | 1.68E−10 |
| 207542_s_at | AQP1 | 1.69421791 | 6.82177348 | 3.02E−12 | 1.81E−10 |
| 242157_at | — | 1.57383098 | 8.76647265 | 3.26E−12 | 1.94E−10 |
| 204058_at | ME1 | 1.73420796 | 9.51508263 | 3.53E−12 | 2.08E−10 |
| 209921_at | SLC7A11 | 1.94348494 | 9.29226579 | 3.63E−12 | 2.13E−10 |
| 211030_s_at | SLC6A6 | 1.51035569 | 7.57360907 | 3.78E−12 | 2.21E−10 |
| 209392_at | ENPP2 | 1.82310668 | 5.23034508 | 4.76E−12 | 2.73E−10 |
| 243894_at | SLC41A2 | 1.69547735 | 5.15085274 | 4.80E−12 | 2.75E−10 |
| 229937_x_at | LILRB1 | 1.58545389 | 7.41501062 | 5.00E−12 | 2.84E−10 |
| 214857_at | C10ORF95 | 1.60255368 | 7.13065345 | 5.02E−12 | 2.85E−10 |
| 230944_at | MGC45491 | 1.55203348 | 7.47887362 | 5.53E−12 | 3.14E−10 |
| 228057_at | DDIT4L | 1.68290143 | 9.61033195 | 6.99E−12 | 3.82E−10 |
| 204105_s_at | NRCAM | 2.27634648 | 6.95288228 | 7.49E−12 | 4.07E−10 |
| 205681_at | BCL2A1 | 1.64702983 | 9.50173198 | 8.19E−12 | 4.40E−10 |
| 242405_at | MAML2 | 1.86052011 | 7.15057318 | 1.10E−11 | 5.73E−10 |
| 217678_at | SLC7A11 | 1.75913555 | 8.8692241 | 1.22E−11 | 6.25E−10 |
| 211962_s_at | ZFP36L1 | 1.59912563 | 8.8232205 | 1.23E−11 | 6.27E−10 |
| 221060_s_at | TLR4 | 1.53812487 | 8.81340284 | 2.27E−11 | 1.08E−09 |
| 200907_s_at | PALLD | 1.50739688 | 7.44132987 | 2.28E−11 | 1.08E−09 |
| 228918_at | SLC43A2 | 1.66364865 | 7.20166079 | 2.29E−11 | 1.09E−09 |
| 1555606_a_at | GDPD1 | 1.51121338 | 6.61034166 | 2.39E−11 | 1.13E−09 |
| 228450_at | PLEKHA7 | 1.50941089 | 6.45253703 | 2.70E−11 | 1.26E−09 |
| 229221_at | — | 1.60263592 | 8.59003199 | 4.03E−11 | 1.79E−09 |
| 229635_at | LOC643424 | 1.53074512 | 7.55215178 | 4.38E−11 | 1.94E−09 |
| 1569149_at | PDLIM7 | 1.54849621 | 9.14969141 | 5.56E−11 | 2.41E−09 |
| 207433_at | IL10 | 1.52400243 | 5.63708634 | 7.49E−11 | 3.14E−09 |
| 221815_at | ABHD2 | 1.55904737 | 8.11542769 | 1.01E−10 | 4.08E−09 |
| 203060_s_at | PAPSS2 | 1.5764103 | 7.75963014 | 1.25E−10 | 4.94E−09 |
| 232746_at | CXCR7 | 2.0719893 | 6.54641126 | 1.43E−10 | 5.55E−09 |
| 1560960_at | MDGA1 | 1.97885133 | 5.71541524 | 1.47E−10 | 5.70E−09 |
| 234645_at | MAML2 | 1.87005824 | 6.53786268 | 1.72E−10 | 6.50E−09 |
| 223596_at | SLC12A6 | 1.50709112 | 7.32492551 | 1.75E−10 | 6.60E−09 |
| 87100_at | ABHD2 | 1.51939681 | 7.38326685 | 1.79E−10 | 6.75E−09 |
| 213931_at | ID2 /// ID2B | 2.71874933 | 9.84931953 | 1.87E−10 | 7.02E−09 |
| 216874_at | DKFZP686O1327 | 1.70886875 | 5.93467446 | 2.11E−10 | 7.76E−09 |
| 244375_at | EVL | 1.56523772 | 7.93537524 | 2.24E−10 | 8.16E−09 |
| 1569150_x_at | PDLIM7 | 1.54117409 | 9.37943359 | 2.24E−10 | 8.18E−09 |
| 214453_s_at | IFI44 | 1.79674041 | 6.65852583 | 2.78E−10 | 9.87E−09 |
| 204999_s_at | ATF5 | 1.64452594 | 9.14215678 | 3.41E−10 | 1.18E−08 |
| 215836_s_at | PCDHGC3 /// PCDHGB4 /// PCDHGA8 | 1.52441384 | 9.29079122 | 4.72E−10 | 1.60E−08 |
| | /// PCDHGA12 /// PCDHGC5 /// | | | | |
| | PCDHGC4 /// PCDHGB7 /// PCDHGB6 | | | | |
| | /// PCDHGB5 /// PCDHGB3 /// | | | | |
| | PCDHGB2 /// PCDHGB1 /// | | | | |
| | PCDHGA11 /// PCDHGA10 /// | | | | |
| | PCDHGA9 /// PCDHGA7 /// PCDHGA6 | | | | |
| | /// PCDHGA5 /// PCDHGA4 /// | | | | |
| | PCDHGA3 /// PCDHGA2 /// PCDHGA1 | | | | |
| 208161_s_at | ABCC3 | 1.61726316 | 8.40189868 | 5.05E−10 | 1.69E−08 |
| 202827_s_at | MMP14 | 1.62314882 | 10.2030389 | 5.39E−10 | 1.78E−08 |
| 208712_at | CCND1 | 1.5393933 | 7.40438263 | 6.16E−10 | 2.01E−08 |
| 223939_at | SUCNR1 | 1.57537424 | 8.40597144 | 7.17E−10 | 2.32E−08 |
| 205960_at | PDK4 | 1.69465703 | 7.19094792 | 1.28E−09 | 3.84E−08 |
| 214841_at | CNIH3 | 1.56253317 | 6.14111085 | 1.40E−09 | 4.15E−08 |
| 209993_at | ABCB1 | 1.58407983 | 5.06880643 | 1.52E−09 | 4.48E−08 |
| 1557938_s_at | PTRF | 1.50339198 | 6.8700736 | 1.62E−09 | 4.73E−08 |
| 232068_s_at | TLR4 | 1.74396268 | 6.76798691 | 1.84E−09 | 5.28E−08 |
| 242321_at | — | 1.6662739 | 6.52856887 | 2.12E−09 | 5.99E−08 |
| 219574_at | MARCH1 | 1.64000788 | 5.55000344 | 3.54E−09 | 9.43E−08 |
| 202638_s_at | ICAM1 | 1.58753294 | 6.72394157 | 3.76E−09 | 9.97E−08 |
| 219496_at | ANKRD57 | 1.50970429 | 7.47583744 | 4.86E−09 | 1.26E−07 |
| 1555756_a_at | CLEC7A | 1.65932872 | 7.0882743 | 5.25E−09 | 1.35E−07 |
| 241929_at | CD36 | 1.82152709 | 8.32014251 | 5.66E−09 | 1.44E−07 |
| 1556423_at | VASH1 | 1.55220936 | 7.02874938 | 8.01E−09 | 1.97E−07 |
| 229435_at | GLIS3 | 1.5824943 | 4.92660385 | 8.07E−09 | 1.98E−07 |
| 217997_at | PHLDA1 | 2.01871208 | 7.37991533 | 8.18E−09 | 2.00E−07 |
| 235944_at | HMCN1 | 1.51434592 | 7.331681 | 9.15E−09 | 2.21E−07 |
| 206637_at | P2RY14 | 1.69022854 | 4.58701985 | 9.57E−09 | 2.30E−07 |
| 213293_s_at | TRiM22 | 1.54165851 | 6.59931282 | 9.57E−09 | 2.30E−07 |
| 238581_at | GBP5 | 1.57383447 | 7.67594481 | 1.03E−08 | 2.46E−07 |
| 1558404_at | LOC644242 /// LOC650429 /// | 1.85121181 | 5.31545142 | 1.46E−08 | 3.35E−07 |
| | LOC650446 | | | | |
| 230559_x_at | FGD4 | 1.51987215 | 5.45151267 | 1.69E−08 | 3.83E−07 |
| 1553151_at | ATP6V0D2 | 1.52950999 | 6.78084809 | 1.69E−08 | 3.84E−07 |
| 1554285_at | HAVCR2 | 1.64907555 | 7.49492704 | 3.00E−08 | 6.38E−07 |
| 244579_at | TRPS1 | 1.5354261 | 8.45494277 | 5.93E−08 | 1.16E−06 |
| 228640_at | — | 1.5809974 | 4.58101232 | 7.52E−08 | 1.43E−06 |
| 210004_at | OLR1 | 1.59400178 | 5.26939608 | 8.40E−08 | 1.58E−06 |
| 243556_at | NGEF | 1.50840974 | 6.73352627 | 1.18E−07 | 2.14E−06 |
| 210360_s_at | MTSS1 | 1.7452905 | 6.18923946 | 1.40E−07 | 2.49E−06 |
| 217999_s_at | PHLDA1 | 1.73310668 | 6.08679858 | 2.07E−07 | 3.52E−06 |
| 227062_at | TNCRNA | 1.56840333 | 10.0441571 | 2.18E−07 | 3.69E−06 |
| 237904_at | ADORA3 | 1.52521897 | 6.15098521 | 2.49E−07 | 4.15E−06 |
| 1563621_at | KIAA1706 | 1.55572929 | 7.60284745 | 6.48E−07 | 9.74E−06 |

Down regulated:

| | | | | | |
|---|---|---|---|---|---|
| 207725_at | POU4F2 | −2.8001105 | 9.19852692 | 2.12E−22 | 1.81E−19 |
| 212993_at | — | −1.8809512 | 9.86599984 | 2.25E−22 | 1.89E−19 |
| 211421_s_at | RET | −2.0125505 | 9.36968529 | 3.53E−22 | 2.84E−19 |
| 227899_at | VIT | −1.8754322 | 7.70929876 | 4.25E−22 | 3.36E−19 |
| 225224_at | C20ORF112 | −2.04379 | 8.5964785 | 1.37E−21 | 8.79E−19 |
| 213361_at | TDRD7 | −1.8488643 | 10.2732718 | 2.93E−21 | 1.67E−18 |
| 214539_at | SERPINB10 | −2.1313083 | 8.01802953 | 3.16E−21 | 1.78E−18 |
| 235275_at | OXCT2 | −2.0229254 | 12.4009174 | 4.06E−21 | 2.20E−18 |
| 203685_at | BCL2 | −2.3112793 | 8.44431889 | 4.88E−21 | 2.59E−18 |
| 227037_at | LOC201164 | −1.5468442 | 9.38990462 | 5.65E−21 | 2.94E−18 |
| 219714_s_at | CACNA2D3 | −2.206206 | 9.82724428 | 5.72E−21 | 2.95E−18 |
| 206643_at | HAL | −1.9307917 | 9.28853464 | 6.18E−21 | 3.10E−18 |
| 211144_x_at | TRGC2 /// TRGV2 /// TRGV9 /// TARP | −1.5748474 | 10.586818 | 7.12E−21 | 3.54E−18 |
| | /// LOC642083 | | | | |
| 217521_at | — | −1.7887534 | 8.38380263 | 7.38E−21 | 3.63E−18 |
| 208206_s_at | RASGRP2 | −1.9706562 | 9.9264082 | 1.25E−20 | 5.95E−18 |
| 228977_at | IL17D | −2.1132305 | 7.51309566 | 2.19E−20 | 1.01E−17 |
| 209813_x_at | TRGC2 /// TRGV2 /// TRGV9 /// TARP | −1.6036338 | 10.7697475 | 3.87E−20 | 1.65E−17 |
| | /// LOC642083 | | | | |
| 204614_at | SERPINB2 | −2.3610663 | 10.8353035 | 6.10E−20 | 2.57E−17 |
| 212110_at | SLC39A14 | −1.682778 | 10.5673949 | 7.23E−20 | 3.00E−17 |
| 202444_s_at | SPFH1 | −1.8415073 | 9.34800962 | 7.37E−20 | 3.03E−17 |
| 216920_s_at | TRGC2 /// TRGV2 /// TRGV9 /// TARP | −1.5704575 | 11.3491957 | 8.78E−20 | 3.56E−17 |
| | /// LOC642083 | | | | |
| 202441_at | SPFH1 | −1.6532676 | 11.1411892 | 1.45E−19 | 5.66E−17 |
| 211919_s_at | CXCR4 | −1.5387599 | 11.0738986 | 3.40E−19 | 1.25E−16 |
| 204610_s_at | CCDC85B | −1.729942 | 9.65009386 | 6.61E−19 | 2.20E−16 |
| 200999_s_at | CKAP4 | −1.6192589 | 11.5934013 | 8.23E−19 | 2.64E−16 |
| 207865_s_at | BMP8B | −2.145585 | 9.11410521 | 9.38E−19 | 2.90E−16 |
| 217028_at | CXCR4 | −1.5274428 | 11.5438714 | 1.14E−18 | 3.50E−16 |
| 201968_s_at | PGM1 | −1.5051483 | 9.06546368 | 2.12E−18 | 6.10E−16 |
| 219463_at | C20ORF103 | −1.5731957 | 10.0186106 | 2.55E−18 | 7.26E−16 |
| 212242_at | TUBA1 | −1.5626324 | 11.2879097 | 2.82E−18 | 7.96E−16 |
| 213484_at | — | −1.5736546 | 8.53538561 | 3.16E−18 | 8.83E−16 |
| 209201_x_at | CXCR4 | −1.5448129 | 11.1194624 | 3.16E−18 | 8.83E−16 |
| 218858_at | DEPDC6 | −1.7866919 | 7.55045943 | 5.06E−18 | 1.34E−15 |
| 202236_s_at | SLC16A1 | −1.5384699 | 10.1675739 | 9.26E−18 | 2.30E−15 |
| 208158_s_at | OSBPL1A | −1.6142678 | 8.3746552 | 1.71E−17 | 4.06E−15 |
| 202932_at | YES1 | −1.6455703 | 8.57014581 | 2.47E−17 | 5.62E−15 |
| 243209_at | KCNQ4 | −2.2463943 | 6.9665965 | 2.56E−17 | 5.79E−15 |
| 215806_x_at | TRGC2 /// TRGV2 /// TRGV9 /// TARP | −1.5328764 | 11.0634752 | 3.51E−17 | 7.61E−15 |
| | /// LOC642083 | | | | |
| 204521_at | C12ORF24 | −1.5676281 | 10.1844282 | 3.88E−17 | 8.36E−15 |
| 204301_at | KBTBD11 | −1.5574513 | 10.253905 | 4.37E−17 | 9.27E−15 |
| 227920_at | KIAA1553 | −1.6307063 | 8.01525239 | 5.17E−17 | 1.09E−14 |
| 218251_at | MID1IP1 | −1.559714 | 9.78570246 | 5.29E−17 | 1.11E−14 |
| 212646_at | RFTN1 | −1.658315 | 8.57843565 | 5.38E−17 | 1.12E−14 |
| 222799_at | HSPC049 | −1.5489442 | 8.60085628 | 1.10E−16 | 2.17E−14 |
| 227103_s_at | MGC2408 | −1.581709 | 8.7267591 | 1.30E−16 | 2.51E−14 |
| 206067_s_at | WT1 | −1.511783 | 9.85618462 | 1.37E−16 | 2.66E−14 |
| 214369_s_at | RASGRP2 | −1.5808582 | 9.41805033 | 1.69E−16 | 3.15E−14 |
| 206589_at | GFI1 | −1.5996464 | 11.7281473 | 1.99E−16 | 3.65E−14 |
| 225619_at | SLAIN1 | −2.2453889 | 7.41012008 | 2.01E−16 | 3.68E−14 |
| 212660_at | PHF15 | −1.69531 | 8.00752875 | 2.63E−16 | 4.66E−14 |
| 218971_s_at | HSPC049 | −1.5087203 | 9.12721477 | 3.08E−16 | 5.33E−14 |
| 201690_s_at | TPD52 | −1.5492402 | 9.27038373 | 3.25E−16 | 5.59E−14 |
| 229638_at | IRX3 | −1.6570492 | 11.8844594 | 3.38E−16 | 5.76E−14 |
| 223062_s_at | PSAT1 | −1.556602 | 9.43823571 | 4.70E−16 | 7.77E−14 |
| 225510_at | OAF | −1.6632727 | 10.1142626 | 6.67E−16 | 1.05E−13 |
| 209485_s_at | OSBPL1A | −1.6049308 | 8.36910929 | 7.34E−16 | 1.15E−13 |
| 204432_at | SOX12 | −1.7584476 | 6.5781916 | 8.71E−16 | 1.33E−13 |
| 205768_s_at | SLC27A2 | −1.6303006 | 7.69181894 | 8.98E−16 | 1.36E−13 |
| 211576_s_at | SLC19A1 | −1.5339487 | 9.8247482 | 9.81E−16 | 1.46E−13 |
| 201688_s_at | TPD52 | −1.6048433 | 8.65393713 | 9.93E−16 | 1.48E−13 |
| 50314_i_at | C20ORF27 | −1.561172 | 9.67877203 | 1.14E−15 | 1.66E−13 |
| 1553436_at | MUC19 | −1.64037 | 7.99717726 | 1.18E−15 | 1.71E−13 |
| 202800_at | SLC1A3 | −1.5176496 | 8.18097955 | 1.51E−15 | 2.13E−13 |
| 41037_at | TEAD4 | −1.6405647 | 6.31029525 | 1.70E−15 | 2.36E−13 |
| 216953_s_at | WT1 | −1.5276508 | 7.76890727 | 2.15E−15 | 2.95E−13 |
| 228055_at | NAPSB | −1.5385754 | 10.1648177 | 2.61E−15 | 3.52E−13 |
| 218424_s_at | STEAP3 | −1.7692882 | 7.88028032 | 2.78E−15 | 3.74E−13 |
| 212855_at | DCUN1D4 | −1.508562 | 7.88887861 | 2.92E−15 | 3.90E−13 |
| 202933_s_at | YES1 | −1.5437793 | 9.12374841 | 3.76E−15 | 4.89E−13 |
| 1553138_a_at | ANKRD41 | −2.0146608 | 6.96935627 | 6.26E−15 | 7.73E−13 |
| 227242_s_at | EBF3 | −1.8363477 | 4.86769268 | 8.15E−15 | 9.84E−13 |
| 237265_at | C16ORF73 | −1.5296704 | 9.74165113 | 9.60E−15 | 1.13E−12 |
| 213478_at | KIAA1026 | −1.8330762 | 7.39399329 | 1.16E−14 | 1.34E−12 |
| 225081_s_at | CDCA7L | −1.5491727 | 10.2951966 | 1.46E−14 | 1.66E−12 |
| 211299_s_at | FLOT2 | −1.6145204 | 9.10864173 | 1.58E−14 | 1.79E−12 |
| 213568_at | OSR2 | −2.2462672 | 5.99962469 | 1.64E−14 | 1.83E−12 |
| 227662_at | SYNPO2 | −1.5973696 | 8.62811124 | 1.76E−14 | 1.96E−12 |
| 200998_s_at | CKAP4 | −1.6910951 | 9.77491302 | 2.08E−14 | 2.25E−12 |
| 209900_s_at | SLC16A1 | −1.5885969 | 7.84437981 | 2.30E−14 | 2.43E−12 |
| 229084_at | CNTN4 | −1.6415136 | 7.72735571 | 2.30E−14 | 2.43E−12 |
| 238694_at | — | −1.5762461 | 7.26420148 | 3.38E−14 | 3.46E−12 |
| 1555788_a_at | TRIB3 | −1.5022329 | 9.92495817 | 3.64E−14 | 3.70E−12 |
| 221900_at | COL8A2 | −1.5627579 | 10.2358313 | 4.09E−14 | 4.05E−12 |
| 225016_at | APCDD1 | −1.6872345 | 6.47036912 | 4.21E−14 | 4.14E−12 |
| 225768_at | NR1D2 | −1.7351352 | 6.8044854 | 5.44E−14 | 5.24E−12 |
| 222095_s_at | C1ORF76 | −1.7301956 | 7.38672115 | 6.40E−14 | 6.00E−12 |
| 229103_at | WNT3 | −1.7749895 | 5.55503782 | 7.97E−14 | 7.21E−12 |
| 205769_at | SLC27A2 | −1.7824201 | 7.67753108 | 1.53E−13 | 1.27E−11 |
| 201689_s_at | TPD52 | −1.645479 | 8.12663653 | 1.59E−13 | 1.31E−11 |
| 201350_at | FLOT2 | −1.5079906 | 11.0344892 | 2.08E−13 | 1.68E−11 |
| 221648_s_at | C1ORF121 | −1.517313 | 8.36815023 | 2.55E−13 | 2.00E−11 |
| 208078_s_at | SNF1LK | −1.597736 | 7.64505173 | 2.81E−13 | 2.18E−11 |
| 1562484_at | FLJ35848 | −1.9050209 | 6.65405977 | 3.15E−13 | 2.41E−11 |
| 1555370_a_at | CAMTA1 | −1.7040191 | 6.36041757 | 3.26E−13 | 2.47E−11 |
| 218376_s_at | MICAL1 | −1.5055354 | 11.194367 | 3.44E−13 | 2.61E−11 |
| 204567_s_at | ABCG1 | −1.8330717 | 5.8226155 | 3.51E−13 | 2.66E−11 |
| 39966_at | CSPG5 | −1.6105172 | 7.68266153 | 4.12E−13 | 3.07E−11 |
| 218326_s_at | LGR4 | −1.696564 | 6.525917 | 5.28E−13 | 3.85E−11 |
| 212430_at | RBM38 | −1.5884137 | 8.88476901 | 5.78E−13 | 4.17E−11 |
| 227236_at | TSPAN2 | −1.7695047 | 6.98434119 | 7.37E−13 | 5.19E−11 |
| 204794_at | DUSP2 | −1.5957268 | 8.45706393 | 7.96E−13 | 5.57E−11 |
| 230888_at | HSPC049 | −1.577802 | 7.20584208 | 9.14E−13 | 6.29E−11 |
| 212097_at | CAV1 | −1.6628831 | 7.59130877 | 9.63E−13 | 6.57E−11 |
| 209353_s_at | C1ORF76 | −1.7131519 | 7.30778535 | 1.02E−12 | 6.92E−11 |
| 201801_s_at | SLC29A1 | −1.6246978 | 8.73200853 | 1.05E−12 | 7.08E−11 |
| 210279_at | GPR18 | −1.5350315 | 8.98079125 | 1.29E−12 | 8.49E−11 |
| 52651_at | COL8A2 | −1.577706 | 9.9516637 | 1.31E−12 | 8.57E−11 |
| 235758_at | PNMA6A | −1.5784572 | 7.86091427 | 1.34E−12 | 8.77E−11 |
| 222162_s_at | ADAMTS1 | −1.842254 | 6.45805339 | 1.37E−12 | 8.95E−11 |
| 209369_at | ANXA3 | −1.7449323 | 5.34631362 | 1.41E−12 | 9.18E−11 |
| 219911_s_at | SLCO4A1 | −1.5143987 | 8.17455361 | 1.50E−12 | 9.71E−11 |
| 1557919_a_at | LOC648232 | −1.5028156 | 12.5466889 | 1.73E−12 | 1.11E−10 |
| 219497_s_at | BCL11A | −1.5825853 | 9.14635109 | 1.85E−12 | 1.18E−10 |
| 232271_at | HNF4G | −1.5978388 | 7.3715225 | 2.04E−12 | 1.29E−10 |
| 206653_at | — | −1.5611734 | 6.70426513 | 2.13E−12 | 1.33E−10 |
| 225782_at | MSRB3 | −1.5020089 | 8.17316381 | 2.54E−12 | 1.55E−10 |
| 223704_s_at | DMRT2 | −1.6370445 | 6.61601092 | 5.67E−12 | 3.20E−10 |
| 1558613_at | OAF | −1.5776535 | 7.5183993 | 6.15E−12 | 3.44E−10 |
| 1555434_a_at | SLC39A14 | −1.5483414 | 7.91388969 | 6.58E−12 | 3.65E−10 |
| 1556194_a_at | — | −1.7264827 | 7.80933003 | 1.10E−11 | 5.73E−10 |
| 219304_s_at | PDGFD | −1.5313053 | 8.14849618 | 1.10E−11 | 5.73E−10 |
| 200894_s_at | FKBP4 | −1.6280907 | 8.06454238 | 1.28E−11 | 6.49E−10 |
| 210347_s_at | BCL11A | −1.5419287 | 7.82101671 | 1.36E−11 | 6.85E−10 |
| 221249_s_at | FAM117A | −1.5920148 | 8.42643515 | 1.45E−11 | 7.21E−10 |
| 239410_at | — | −1.521547 | 6.86497836 | 1.72E−11 | 8.46E−10 |
| 206039_at | RAB33A | −1.5487461 | 8.85562976 | 2.20E−11 | 1.05E−09 |
| 1554660_a_at | C1ORF71 | −1.5203911 | 7.61313106 | 2.54E−11 | 1.19E−09 |
| 209776_s_at | SLC19A1 | −1.5451778 | 7.72495148 | 2.90E−11 | 1.34E−09 |
| 1560495_at | — | −1.6478409 | 7.28259363 | 3.01E−11 | 1.386−09 |
| 202234_s_at | SLC16A1 | −1.5891823 | 7.76240785 | 4.97E−11 | 2.16E−09 |
| 207768_at | EGR4 | −2.266679 | 5.08097601 | 7.18E−11 | 3.02E−09 |
| 207717_s_at | PKP2 | −1.5155863 | 4.5970225 | 1.26E−10 | 4.97E−09 |
| 213912_at | KIAA0984 | −1.5617265 | 6.49154577 | 1.30E−10 | 5.13E−09 |
| 222891_s_at | BCL11A | −1.5929549 | 8.42933212 | 2.00E−10 | 7.44E−09 |
| 213268_at | CAMTA1 | −1.7460973 | 5.75010725 | 2.03E−10 | 7.52E−09 |
| 232007_at | — | −1.5199738 | 9.70608088 | 2.60E−10 | 9.33E−09 |
| 227099_s_at | LOC387763 | −1.5087957 | 6.4279952 | 2.97E−10 | 1.05E−08 |
| 232614_at | BCL2 | −1.6221488 | 7.30276639 | 2.99E−10 | 1.05E−08 |
| 222668_at | KCTD15 | −1.5400712 | 5.79509816 | 3.31E−10 | 1.15E−08 |
| 213610_s_at | KLHL23 | −1.5730988 | 7.2326013 | 4.60E−10 | 1.56E−08 |
| 206935_at | PCDH8 | −1.6992238 | 5.66380311 | 6.65E−10 | 2.16E−08 |
| 203708_at | PDE4B | −1.5144333 | 7.44164625 | 8.10E−10 | 2.59E−08 |
| 1554298_a_at | WDR49 | −1.5642625 | 6.77053726 | 8.63E−10 | 2.73E−08 |
| 212558_at | SPRY1 | −1.6087624 | 5.95823849 | 1.41E−09 | 4.18E−08 |
| 242509_at | C16ORF74 | −1.5318238 | 6.48465026 | 2.21E−09 | 6.20E−08 |
| 201939_at | PLK2 | −1.5317787 | 7.43694803 | 5.43E−09 | 1.39E−07 |
| 242245_at | SYDE2 | −1.5851454 | 4.93022084 | 5.57E−09 | 1.42E−07 |
| 204557_s_at | DZIP1 | −1.5326546 | 5.01217049 | 5.85E−09 | 1.48E−07 |
| 204875_s_at | GMDS | −1.7607496 | 6.33236847 | 9.81E−09 | 2.35E−07 |
| 204285_s_at | PMAIP1 | −1.7424137 | 10.9563243 | 1.15E−08 | 2.73E−07 |
| 213006_at | CEBPD | −1.9902998 | 8.08295825 | 1.47E−08 | 3.38E−07 |
| 1554830_a_at | STEAP3 | −1.6156641 | 5.65856155 | 2.19E−08 | 4.84E−07 |
| 204286_s_at | PMAIP1 | −1.7353429 | 9.45448346 | 6.05E−08 | 1.18E−06 |
| 228325_at | KIAA0146 | −1.8819676 | 8.36526925 | 8.31E−08 | 1.57E−06 |
| 1569377_at | TMEM67 | −1.9524092 | 4.4888277 | 1.11E−07 | 2.04E−06 |
| 242892_at | PER2 | −2.1304461 | 6.97699912 | 1.72E−07 | 3.00E−06 |
| 1555847_a_at | LOC84454 | −2.1798122 | 9.88866319 | 1.88E−07 | 3.24E−06 |

### Discussion

### Example 1

The significant gene-expression changes observed in the human THP-1 cell line due to treatment with branded GA included changes consistent with previous literature (as discussed below), supporting the validity of the chosen model system and current study design for revealing relevant treatment effects.

For example, expression of the anti-inflammatory gene IL10 was increased at the 6 hour timepoint, consistent with known GA mechanism with regard to monocytes (Kim et al. J. Immunol. Baltim. Md 1950, 2004; Weber et al. Nat. Med. 2007; Mahad et al. Brain J. Neurol. 2006). GA is thought to induce an anti-inflammatory effect, mediated by secretion of IL-4, IL-10, and other anti inflammatory cytokines both in terms of T cells (Th1 to Th2 shift) but also in terms of monocytes, resulting in a shift from monocyte production of IL-12 to anti-inflammatory IL-10. For example, monocytes from mice treated with GA secreted more IL-10 than monocytes from untreated mice (Weber, Nat Med 2007), and monocytes isolated from MS patients treated with GA were shown to produce more IL-10 relative to untreated patients (Kim, J Immunol 2004). In addition, dendritic cells exposed to GA during maturation increased their production of IL-10 (Mahad et al. Brain J. Neurol. 2006).

Another anti-inflammatory gene, IL1RN (encoding IL-1ra, a protein that inhibits the activities of IL-1a and IL-1b) showed increased expression at all three timepoints. These observations are consistent with work showing that blood levels of soluble IL1-ra increase with GA treatment in patients with MS as well as EAE mice, and that soluble IL1-ra is upregulated by GA treatment in human monocytes either stimulated with LPS or activated by T cell contact (Burger et al, PNAS 2009).

Branded GA significantly modulated many pathways (Table 5). At 6h, pathways enriched significantly among upregulated genes included broad categories such as immune response and regulation of immune processes, and more specifically cytokine-cytokine receptor interactions. Other significantly enriched pathways included adhesion, and other pathways with broad relevance to the disease process and/or proposed action of GA. Several of these pathways were also significantly enriched among genes modulated by GA in monocytes from RRMS patients (Rosenberg, The Lancet 2005).

### Example 2

### Differences in Gene Expression Induced by Probioglat Versus Branded GA

To identify differences between branded GA and differently manufactured glatiramoids, differential gene expression analysis was performed to compare directly between profiles induced by branded GA and by the purported generic glatiramoid, Probioglat. The standard R LIMMA bioconductor package was utilized to measure differentially expressed probesets across the entire microarray. To compare GA and Probioglat, comparisons were corrected to compare each treatment relative to mannitol control (i.e., [GA vs mannitol] was compared via LIMMA to [Probioglat vs mannitol]). Probesets were filtered by calls of presence on the chip for the relevant samples in the comparison (to be considered present at a given timepoint, a probeset was required to have on average a call of present or marginal across the relevant samples at that timepoint). Probesets were mapped to genes using the annotation available for the U133 Plus 2.0 chip from Affymetrix. FDR adjusted p values reported for genes represent the lowest FDR adjusted p value for present probesets for that gene.

Many significant differences were observed between GA and Probioiglat (Table 3). As expected based on the more extensive response to GA at 6h, the most differences were observed at the 6h timepoint.

TABLE 3
Dynamic profiles of differentially expressed RNA after stimulation
of THP-1 cells by Probioglat versus Copaxone ®

| | | Stimulation time | | | | | |
|---|---|---|---|---|---|---|---|
| | | 6 hours | | 12 hours | | 24 hours | |
| | Significance | Genes | Probesets | Genes | Probesets | Genes | Probesets |
| | threshold | # | # | # | # | # | # |
| Upregulated: | FDR- | 115 | 138 | 5 | 5 | 1 | 1 |
| | adjusted p | | | | | (MMP9) | |
| | value < 0.05 | | | | | | |
| | Nominal | 2,597 | 3,310 | 1,296 | 1,560 | 1,625 | 1,959 |
| | p value < 0.05 | | | | | | |
| Total | FDR- | 136 | 162 | 7 | 7 | 1 | 1 |
| modulated | adjusted p | | | | | (MMP9) | |
| (up- and | value < 0.05 | | | | | | |
| down- | Nominal | 4,863 | 6,208 | 3,051 | 3,992 | 2,843 | 3,486 |
| regulated): | p value < 0.05 | | | | | | |

Numbers of genes and probesets modulated by Probioglat relative to GA

(percent of probesets detected as significantly differentiated between treatments as percentage of the total 47,000 probesets included in the Affymetrix U133 Plus 2.0 chip)

See Table 4a for the full list of differentially-expressed probesets at 6h: 138 upregulated, 24 downregulated (126 upregulated, 22 downregulated after presence/absence filtering). These differences included proinflammatory genes that were increased in expression by Probioglat relative to GA, including CCL5, CCL2, MMP9, MMP1, CXCL10, CD14, ICAM1 and BIRC3 (all significant by FDR adjusted p value<0.05) (Table 4a). At the same time, differences were observed in levels of anti-inflammatory genes. Probioglat downregulated anti-inflammatory genes CISH and HSPD1 relative to GA, and upregulated IL10 and PRDM1 relative to branded GA (all significant by FDR adjusted p value<0.05).

TABLE 4a
Human monocyte study: probesets significantly modulated by Copaxone ® relative to Probioglat
Comparing GA to Probioglat (mannitol-corrected):

| | | | | | | | |
|---|---|---|---|---|---|---|---|
| Gene | ID | raw. FC | AveExpr | t | P. Value | adj. P. Val | B |

Upregulated:
6 hr:

| | | | | | | | |
|---|---|---|---|---|---|---|---|
| MMP9 | 203936_s_at | −1.2926295 | 9.63366142 | −9.0041791 | 5.02E−11 | 2.74E−06 | 13.793663 |
| CXCL10 | 204533_at | −1.4567769 | 5.93611913 | −6.8101441 | 4.11E−08 | 0.00056208 | 8.11662055 |
| PRDM1 | 228964_at | −1.3131543 | 9.26923221 | −6.9712916 | 2.48E−08 | 0.00056208 | 8.55501455 |
| LPXN | 216250_s_at | −1.1874257 | 11.315416 | −6.8905168 | 3.19E−08 | 0.00056208 | 8.33560319 |
| FABP4 | 203980_at | −1.3897081 | 8.03922119 | −6.4609935 | 1.24E−07 | 0.00100306 | 7.15838496 |
| — | 240076_at | −1.2154985 | 8.47153306 | −6.4502687 | 1.28E−07 | 0.00100306 | 7.12879005 |
| COL6A1 | 213428_s_at | −1.1799797 | 9.17734162 | −6.5227006 | 1.02E−07 | 0.00100306 | 7.3284926 |
| SLC39A8 | 209267_s_at | −1.1409956 | 10.4538343 | −6.3855772 | 1.58E−07 | 0.00107772 | 6.95009387 |
| MGC5618 | 221477_s_at | −1.1617675 | 10.5471179 | −6.2424898 | 2.48E−07 | 0.00135843 | 6.55382496 |
| SLC39A8 | 219869_s_at | −1.14866 | 10.1877186 | −6.2694824 | 2.28E−07 | 0.00135843 | 6.62867956 |
| — | 226218_at | −1.2937342 | 7.33564432 | −6.1059571 | 3.84E−07 | 0.00140732 | 6.17456221 |
| STEAP1 | 205542_at | −1.198521 | 7.98610593 | −6.0852841 | 4.10E−07 | 0.00140732 | 6.11705174 |
| CHST11 | 219634_at | −1.1616563 | 9.51077621 | −6.0764318 | 4.21E−07 | 0.00140732 | 6.09241933 |
| CD9 | 201005_at | −1.1480015 | 10.9097322 | −6.0774378 | 4.20E−07 | 0.00140732 | 6.09521866 |
| TNF5F13B | 223502_s_at | −1.137131 | 10.6086468 | −6.1814333 | 3.02E−07 | 0.00140732 | 6.38434731 |
| LACTB | 226354_at | −1.1146026 | 10.6969136 | −6.1724213 | 3.10E−07 | 0.00140732 | 6.35931437 |
| ARL6IP5 | 200761_s_at | −1.1108957 | 11.9357889 | −6.0645684 | 4.38E−07 | 0.00140732 | 6.05940234 |
| NFKBIE | 203927_at | −1.1646409 | 10.0523732 | −5.9997498 | 5.38E−07 | 0.00157615 | 5.87889643 |
| SLIC1 | 228869_at | −1.1244476 | 10.0277009 | −5.994027 | 5.48E−07 | 0.00157615 | 5.86295139 |
| MMP1 | 204475_at | −1.5039262 | 6.32353561 | −5.9312361 | 6.69E−07 | 0.00174156 | 5.68791885 |
| SOD2 | 215223_s_at | −1.2035944 | 9.66361398 | −5.9462273 | 6.38E−07 | 0.00174156 | 5.72972041 |
| NFKBIA | 201502_s_at | −1.1701699 | 11.8415908 | −5.9153553 | 7.04E−07 | 0.0017486 | 5.64362855 |
| ADAMDEC1 | 206134_at | −1.1861199 | 8.53747931 | −5.7927992 | 1.04E−06 | 0.00247065 | 5.30158178 |
| MPEG1 | 226841_at | −1.141713 | 9.90354597 | −5.7187643 | 1.32E−06 | 0.00281664 | 5.09479213 |
| ANXA2P2 | 208816_x_at | −1.1086074 | 11.6486476 | −5.7130844 | 1.34E−06 | 0.00281664 | 5.07892372 |
| — | 244434_at | −1.2846909 | 6.48107186 | −5.6742369 | 1.52E−06 | 0.00284109 | 4.97038261 |
| CCL2 | 216598_s_at | −1.2516796 | 11.2421198 | −5.6653825 | 1.56E−06 | 0.00284109 | 4.94564092 |
| ISG20 | 204698_at | −1.2176821 | 6.18754674 | −5.677111 | 1.50E−06 | 0.00284109 | 4.97841335 |
| MTSS1 | 203037_s_at | −1.2132697 | 11.5042636 | −5.6909681 | 1.44E−06 | 0.00284109 | 5.0171321 |
| IFNGR1 | 211676_s_at | −1.1076638 | 12.1480464 | −5.6472459 | 1.65E−06 | 0.0029127 | 4.89496045 |
| — | 230795_at | −1.1702144 | 9.26991641 | −5.6333398 | 1.73E−06 | 0.00294926 | 4.85610012 |
| NFE2L3 | 236471_at | −1.3257415 | 5.26403664 | −5.6026446 | 1.90E−06 | 0.00315306 | 4.77032024 |
| CD36 | 209555_s_at | −1.1414722 | 9.39991418 | −5.5482888 | 2.26E−06 | 0.00363742 | 4.61841752 |
| CYLD | 221903_s_at | −1.1207309 | 9.54523467 | −5.5045621 | 2.60E−06 | 0.00406002 | 4.49622693 |
| ICAM1 | 202638_s_at | −1.4084929 | 6.8285265 | −5.4886851 | 2.73E−06 | 0.00415135 | 4.45186392 |
| KIAA1505 | 227265_at | −1.1673293 | 9.93164349 | −5.4623712 | 2.97E−06 | 0.00423964 | 4.37834442 |
| BID | 227143_s_at | −1.1112086 | 11.5740779 | −5.471422 | 2.89E−06 | 0.00423964 | 4.40363069 |
| BID | 211725_s_at | −1.1017724 | 11.8106494 | −5.4568423 | 3.02E−06 | 0.00423964 | 4.36289801 |
| IL10 | 207433_at | −1.2783883 | 5.71640286 | −5.3801791 | 3.86E−06 | 0.00508408 | 4.14877517 |
| ANXA2 | 201590_x_at | −1.1067663 | 13.1932317 | −5.3762172 | 3.91E−06 | 0.00508408 | 4.13771284 |
| TNFAIP6 | 206026_s_at | −1.2533604 | 5.46505459 | −5.3471287 | 4.28E−06 | 0.00544542 | 4.0565035 |
| CD14 | 201743_at | −1.1688628 | 9.56313815 | −5.3162757 | 4.72E−06 | 0.00586803 | 3.97039297 |
| ICAM1 | 202637_s_at | −1.253703 | 7.7078627 | −5.2602772 | 5.64E−06 | 0.00674579 | 3.81417837 |
| ANXA2 | 210427_x_at | −1.1021711 | 13.1600502 | −5.2581893 | 5.68E−06 | 0.00674579 | 3.80835622 |
| SYNJ2 | 212828_at | −1.1079961 | 9.8266126 | −5.2259269 | 6.29E−06 | 0.00731141 | 3.71841109 |
| GLIPR1 | 226142_at | −1.1476518 | 8.96232728 | −5.2116592 | 6.57E−06 | 0.00744619 | 3.67864746 |
| ECOP | 238604_at | −1.1291845 | 9.93048271 | −5.2069623 | 6.67E−06 | 0.00744619 | 3.66555947 |
| CD40 | 205153_s_at | −1.1516665 | 8.96773222 | −5.1878467 | 7.09E−06 | 0.00775153 | 3.61230284 |
| IFIH1 | 219209_at | −1.1839872 | 8.4216604 | −5.1481808 | 8.03E−06 | 0.0086134 | 3.50184783 |
| IL4I1 | 230966_at | −1.2886142 | 10.7302425 | −5.1035983 | 9.25E−06 | 0.00953971 | 3.3777992 |
| MAFB | 218559_s_at | −1.1661498 | 12.5070822 | −5.0783026 | 1.00E−05 | 0.01013992 | 3.30746517 |
| DAB2 | 201278_at | −1.1002534 | 9.40858428 | −5.0704754 | 1.03E−05 | 0.01020405 | 3.28570957 |
| P2RX4 | 204088_at | −1.11462 | 10.5804136 | −5.0589302 | 1.06E−05 | 0.01039288 | 3.25362692 |
| MLF1 | 204784_s_at | −1.1629248 | 9.99075341 | −5.0382007 | 1.14E−05 | 0.01089897 | 3.19604331 |
| STATH | 206835_at | −1.2217631 | 10.6277925 | −4.9970174 | 1.29E−05 | 0.01198544 | 3.08172746 |
| GIMAP8 | 235306_at | −1.1769391 | 7.15724646 | −4.9534429 | 1.48E−05 | 0.01329267 | 2.96090645 |
| TATDN3 | 235069_at | −1.1552651 | 9.09091742 | −4.9541849 | 1.48E−05 | 0.01329267 | 2.96296263 |
| SYNJ2 | 216180_s_at | −1.1540719 | 8.13588041 | −4.929194 | 1.60E−05 | 0.01372355 | 2.89373383 |
| TREM1 | 219434_at | −1.1280034 | 11.1714599 | −4.9272524 | 1.61E−05 | 0.01372355 | 2.88835722 |
| ANXA2 | 213503_x_at | −1.1003875 | 13.1627385 | −4.8992037 | 1.76E−05 | 0.01434614 | 2.81072248 |
| G0S2 | 213524_s_at | −1.1355006 | 8.75639391 | −4.864242 | 1.96E−05 | 0.01577044 | 2.71404776 |
| IL10RA | 204912_at | −1.1199911 | 10.5172379 | −4.8323374 | 2.17E−05 | 0.01717268 | 2.62592271 |
| BIRC3 | 210538_s_at | −1.257364 | 4.57445587 | −4.8159991 | 2.28E−05 | 0.01765329 | 2.58083111 |
| NT5E | 203939_at | −1.2383789 | 8.45460557 | −4.8143591 | 2.29E−05 | 0.01765329 | 2.57630644 |
| CCL5 | 1555759_a_i | −1.0910656 | 13.6361191 | −4.8032103 | 2.37E−05 | 0.01802488 | 2.54555373 |
| SRPX2 | 205499_at | −1.224512 | 6.16138512 | −4.767627 | 2.65E−05 | 0.01937153 | 2.4474847 |
| ARL6IP5 | 200760_s_at | −1.1107109 | 11.7204466 | −4.7591181 | 2.72E−05 | 0.01959427 | 2.42405329 |
| EBI3 | 219424_at | −1.1775996 | 7.06372735 | −4.7474062 | 2.82E−05 | 0.01976554 | 2.39181393 |
| CD40 | 215346_at | −1.1705995 | 8.77095715 | −4.7439006 | 2.86E−05 | 0.01976554 | 2.38216714 |
| SRA1 | 224130_s_at | −1.094757 | 10.8027383 | −4.7442703 | 2.85E−05 | 0.01976554 | 2.38318441 |
| — | 238501_at | −1.2105582 | 5.44953239 | −4.73925 | 2.90E−05 | 0.01977223 | 2.36937124 |
| SLIC1 | 229045_at | −1.1547886 | 8.27898536 | −4.7357646 | 2.93E−05 | 0.01977223 | 2.35978293 |
| C5ORF13 | 238411_x_at | −1.1683694 | 3.79974787 | −4.7297145 | 2.98E−05 | 0.01979262 | 2.34314234 |
| CARD15 | 220066_at | −1.1393689 | 8.86627619 | −4.7275995 | 3.00E−05 | 0.01979262 | 2.33732587 |
| PLEKHO1 | 218223_s_at | −1.1393641 | 9.19617144 | −4.7232262 | 3.05E−05 | 0.01982503 | 2.32530094 |
| NFE2L3 | 204702_s_at | −1.1949945 | 6.87002251 | −4.7001908 | 3.27E−05 | 0.02069819 | 2.26199661 |
| P2RY5 | 218589_at | −1.1719902 | 9.42743243 | −4.6980902 | 3.29E−05 | 0.02069819 | 2.25622666 |
| P5CDBP | 209606_at | −1.1833853 | 8.14303401 | −4.6940285 | 3.34E−05 | 0.02072301 | 2.24507189 |
| PTX3 | 206157_at | −1.1323376 | 11.2731131 | −4.6853648 | 3.43E−05 | 0.0210494 | 2.22128475 |
| GHRL | 223862_at | −1.184681 | 7.10722772 | −4.6529266 | 3.79E−05 | 0.02252098 | 2.13230051 |
| SGIP1 | 223672_at | −1.1713796 | 8.75400755 | −4.6556052 | 3.76E−05 | 0.02252098 | 2.13964377 |
| RPL13 /// LO [symbol] | 214976_at | −1.1498626 | 6.91302234 | −4.6576183 | 3.73E−05 | 0.02252098 | 2.14516306 |
| C13ORF31 | 1553141_at | −1.2240082 | 8.3027252 | −4.6445617 | 3.89E−05 | 0.02286455 | 2.10937474 |
| — | 228573_at | −1.0954948 | 10.1938031 | −4.631833 | 4.05E−05 | 0.02353205 | 2.07450537 |
| — | 213891_s_at | −1.1613505 | 8.36985954 | −4.6072376 | 4.37E−05 | 0.02435882 | 2.00718586 |
| C9ORF130 | 227893_at | −1.1466602 | 7.47771908 | −4.6095058 | 4.34E−05 | 0.02435882 | 2.01339098 |
| VPS33A | 204590_x_at | −1.1717826 | 7.44063872 | −4.5964894 | 4.51E−05 | 0.0246793 | 1.97779156 |
| LACTB | 1552486_s_at | −1.1146334 | 9.43978855 | −4.5992016 | 4.48E−05 | 0.0246793 | 1.98520746 |
| RAB27B | 228708_at | −1.2146134 | 8.64471961 | −4.5896791 | 4.61E−05 | 0.02495536 | 1.95917448 |
| FXYD2 | 207434_s_at | −1.2391134 | 6.87935784 | −4.5721048 | 4.87E−05 | 0.02609092 | 1.91116075 |
| SOD2 | 216841_s_at | −1.1587002 | 8.29547849 | −4.5601654 | 5.05E−05 | 0.02658149 | 1.87856531 |
| INADL | 214493_s_at | −1.1876014 | 5.13104061 | −4.5382854 | 5.40E−05 | 0.02787086 | 1.81888217 |
| BID | 204493_at | −1.0962323 | 11.1430522 | −4.5406927 | 5.36E−05 | 0.02787086 | 1.82544538 |
| BTG1 | 240347_at | −1.1577464 | 6.46143817 | −4.5117153 | 5.86E−05 | 0.02941834 | 1.74649575 |
| CENTA2 | 219358_s_at | −1.1328376 | 10.4221094 | −4.5125118 | 5.85E−05 | 0.02941834 | 1.74866433 |
| LOC54103 | 213142_x_at | −1.1327449 | 10.7990817 | −4.5010122 | 6.06E−05 | 0.02985684 | 1.71736561 |
| — | 232297_at | −1.1396483 | 8.4593218 | −4.4977208 | 6.12E−05 | 0.02989176 | 1.7084108 |
| THBD | 203887_s_at | −1.0860181 | 12.8670409 | −4.4922535 | 6.23E−05 | 0.02996581 | 1.6935397 |
| KYNU | 210663_s_at | −1.1273404 | 10.5827862 | −4.4730592 | 6.61E−05 | 0.03124079 | 1.64136616 |
| TATDN3 | 228867_at | −1.0979673 | 8.53193411 | −4.4719767 | 6.63E−05 | 0.03124079 | 1.6384252 |
| ITGB5 | 201125_s_at | −1.1305667 | 9.66682508 | −4.4663279 | 6.74E−05 | 0.03151651 | 1.62308225 |
| HNRPLL | 225386_s_at | −1.088481 | 10.9979975 | −4.4607622 | 6.86E−05 | 0.03178871 | 1.60796956 |
| CRYBB2 | 206777_s_at | −1.1273748 | 9.09050704 | −4.4486878 | 7.12E−05 | 0.03248173 | 1.57520029 |
| CD55 | 1555950_a_i | −1.101176 | 11.0430169 | −4.4455718 | 7.19E−05 | 0.03248173 | 1.56674717 |
| MPEG1 | 226818_at | −1.102944 | 9.97050985 | −4.4364675 | 7.39E−05 | 0.03312876 | 1.54205788 |
| POPDC3 | 219926_at | −1.2923154 | 6.73431127 | −4.4296067 | 7.55E−05 | 0.03333429 | 1.52346112 |
| PLAUR | 214866_at | −1.1197536 | 10.1465619 | −4.4291541 | 7.56E−05 | 0.03333429 | 1.52223465 |
| MGLL | 239914_at | −1.1755243 | 5.02263039 | −4.4192631 | 7.79E−05 | 0.03354952 | 1.49543825 |
| EBI2 | 205419_at | −1.1647466 | 10.3787766 | −4.4209004 | 7.75E−05 | 0.03354952 | 1.49987275 |
| TP5AB1 /// T | 207741_x_at | −1.154419 | 7.49088254 | −4.4208416 | 7.76E−05 | 0.03354952 | 1.49971361 |
| SLAMF8 | 219386_s_at | −1.1604525 | 10.1187042 | −4.4123213 | 7.96E−05 | 0.03370605 | 1.47664099 |
| VSNL1 | 203797_at | −1.1465556 | 9.26432896 | −4.4080282 | 8.07E−05 | 0.03370605 | 1.46501964 |
| ICAM2 | 213620_s_at | −1.129714 | 7.80787761 | −4.4083036 | 8.06E−05 | 0.03370605 | 1.46576519 |
| KYNU | 217388_s_at | −1.1268071 | 11.2241113 | −4.4076297 | 8.08E−05 | 0.03370605 | 1.4639412 |
| ARHGAP18 | 225166_at | −1.1854012 | 7.40195979 | −4.3979297 | 8.32E−05 | 0.03445978 | 1.43769541 |
| MITF | 226066_at | −1.1771138 | 7.94053593 | −4.3803753 | 8.78E−05 | 0.03578326 | 1.39023722 |
| MXD1 | 226275_at | −1.1272655 | 8.58697195 | −4.3782779 | 8.84E−05 | 0.03578326 | 1.38457024 |
| — | 241389_at | −1.1324551 | 7.7082004 | −4.3740983 | 8.95E−05 | 0.03597723 | 1.37327956 |
| EGF | 206254_at | −1.2273072 | 5.23328013 | −4.3445051 | 9.80E−05 | 0.03909529 | 1.29342221 |
| MALT1 | 210017_at | −1.1361187 | 7.85916694 | −4.3417635 | 9.88E−05 | 0.03913824 | 1.28603152 |
| AKR1C2 | 211653_x_at | −1.214586 | 9.21551731 | −4.3261491 | 0.00010361 | 0.03933789 | 1.24396403 |
| LPAAT-THETA | 224480_s_at | −1.2033448 | 8.82402466 | −4.3329062 | 0.00010149 | 0.03933789 | 1.26216342 |
| MFI2 | 235911_at | −1.1178645 | 10.2132798 | −4.3339031 | 0.00010118 | 0.03933789 | 1.26484915 |
| ARHGAP18 | 225171_at | −1.1011604 | 10.7603262 | −4.329986 | 0.0001024 | 0.03933789 | 1.25429744 |
| TXNL2 | 209080_x_at | −1.0905642 | 11.1937357 | −4.3267122 | 0.00010343 | 0.03933789 | 1.24548038 |
| CD55 | 201926_s_at | −1.0876428 | 11.0124454 | −4.3312016 | 0.00010202 | 0.03933789 | 1.25757168 |
| ADAM9 | 202381_at | −1.1288776 | 10.068453 | −4.2795249 | 0.00011941 | 0.04461757 | 1.11860933 |
| OS8PL11 | 218304_s_at | −1.1200967 | 8.80861724 | −4.268338 | 0.00012355 | 0.04461757 | 1.08859065 |
| C5ORF32 | 224797_at | −1.1088424 | 9.60174121 | −4.2676443 | 0.00012381 | 0.04461757 | 1.08672987 |
| CD300A | 209933_s_at | −1.0919093 | 11.068246 | −4.2779818 | 0.00011998 | 0.04461757 | 1.11446708 |
| ATP2C1 | 237278_x_at | −1.1252002 | 4.516837 | −4.2604681 | 0.00012654 | 0.04474124 | 1.06748681 |
| TGM5 | 207911_s_at | −1.0985729 | 10.9973837 | −4.255458 | 0.00012848 | 0.04474124 | 1.05405768 |
| CAST | 208908_s_at | −1.0958388 | 10.4613552 | −4.2557149 | 0.00012838 | 0.04474124 | 1.05474617 |
| TNFRSF9 | 207536_s_at | −1.1669411 | 6.64800587 | −4.2459826 | 0.00013222 | 0.04518347 | 1.02867283 |
| HMGB2 | 243368_at | −1.1602025 | 4.88716386 | −4.2463169 | 0.00013209 | 0.04518347 | 1.02956814 |
| TNFAIP3 | 202644_s_at | −1.1253187 | 10.6490533 | −4.2484635 | 0.00013123 | 0.04518347 | 1.03531746 |
| C1ORF21 | 223125_s_at | −1.1101543 | 11.0658204 | −4.2399019 | 0.00013469 | 0.04573895 | 1.01239127 |
| — | 212387_at | −1.148179 | 7.86470579 | −4.2245512 | 0.0001411 | 0.04762227 | 0.97132047 |

12 hr:

| | | | | | | | |
|---|---|---|---|---|---|---|---|
| PRDM1 | 228964_at | −1.3131187 | 7.67111264 | −8.6246024 | 1.41E−10 | 7.72E−06 | 11.3048141 |
| RCSD1 | 239328_at | −1.2917804 | 9.91539671 | −6.452108 | 1.22E−07 | 0.0033307 | 6.31495199 |
| BTG1 | 240347_at | −1.2562187 | 6.63516956 | −5.7047112 | 1.33E−06 | 0.02422499 | 4.46209908 |
| FBXO45 | 225099_at | 1.13350791 | 9.69247626 | 5.51958363 | 2.40E−06 | 0.03282196 | 3.99779146 |
| ZNF566 | 240239_at | 1.20902406 | 6.20206745 | 5.35216774 | 4.09E−06 | 0.04477351 | 3.57707114 |

24 hr:

| | | | | | | | |
|---|---|---|---|---|---|---|---|
| MMP9 | 203936_s_at | −1.2587837 | 8.76550502 | −6.6308842 | 7.89E−08 | 0.00431641 | 6.0372842 |

Downregulated:
6 hr:

| | | | | | | | |
|---|---|---|---|---|---|---|---|
| LOC648342 | 1569392_at | 1.25828385 | 6.46339373 | 5.71764607 | 1.32E−06 | 0.00281664 | 5.09166798 |
| BTBD14A | 243431_at | 1.31196093 | 8.69387594 | 5.37680876 | 3.90E−06 | 0.00508408 | 4.13936456 |
| SERPINB2 | 204614_at | 1.25751853 | 10.7388922 | 5.14040426 | 8.23E−06 | 0.00865761 | 3.48020227 |
| MYB | 204798_at | 1.09897729 | 13.1513017 | 5.00361266 | 1.27E−05 | 0.01194194 | 3.10002645 |
| STT3B | 238303_at | 1.2645975 | 6.00460112 | 4.9254338 | 1.62E−05 | 0.01372355 | 2.88332163 |
| HSPD1 | 241716_at | 1.30689927 | 6.02121553 | 4.9223221 | 1.64E−05 | 0.01372355 | 2.87470616 |
| ACTN4 | 232058_at | 1.19228742 | 8.60657642 | 4.91815847 | 1.66E−05 | 0.01372355 | 2.86317939 |
| FAM62A | 244234_at | 1.14840119 | 7.29749849 | 4.78192868 | 2.54E−05 | 0.01899888 | 2.48688527 |
| HIC2 | 1559600_at | 1.17980654 | 8.38342055 | 4.76703429 | 2.66E−05 | 0.01937153 | 2.4458524 |
| OXCT2 | 235275_at | 1.16642629 | 12.3300201 | 4.71420617 | 3.13E−05 | 0.02014951 | 2.30050545 |
| — | 228907_at | 1.17599277 | 8.21884465 | 4.6120071 | 4.30E−05 | 0.02435882 | 2.02023432 |
| GAPDHS | 222280_at | 1.1646993 | 7.71035699 | 4.60723903 | 4.37E−05 | 0.02435882 | 2.00718978 |
| SPFH1 | 202441_at | 1.12657525 | 11.0883546 | 4.55979394 | 5.06E−05 | 0.02658149 | 1.87755147 |
| NAPB | 1570441_at | 1.20267749 | 5.92073389 | 4.52611168 | 5.61E−05 | 0.02866712 | 1.78570418 |
| HNRPD | 213359_at | 1.16888386 | 9.72519123 | 4.50340998 | 6.02E−05 | 0.02985684 | 1.72389004 |
| CISH | 223377_x_at | 1.09133621 | 9.01105134 | 4.49116936 | 6.25E−05 | 0.02996581 | 1.69059129 |
| SFRS14 | 64371_at | 1.12400512 | 8.1962891 | 4.44643142 | 7.17E−05 | 0.03248173 | 1.5690791 |
| C14ORF10 | 239188_at | 1.16314645 | 6.54891766 | 4.38855767 | 8.56E−05 | 0.03519643 | 1.4123519 |
| — | 232903_at | 1.21778341 | 6.93050721 | 4.29620676 | 0.0001135 | 0.04279881 | 1.16341547 |
| HDAC4 | 232225_at | 1.14445571 | 7.77327378 | 4.27489999 | 0.00012111 | 0.04461757 | 1.10619611 |
| CLK1 | 214683_s_at | 1.160507 | 9.27028645 | 4.268656 | 0.00012343 | 0.04461757 | 1.08944366 |
| CRLF3 | 235803_at | 1.19940674 | 9.07113133 | 4.2670238 | 0.00012404 | 0.04461757 | 1.08506571 |
| ZNF250 | 241738_at | 1.11389598 | 4.2390353 | 4.26401964 | 0.00012518 | 0.04473242 | 1.07700912 |
| WDFY1 | 234157_at | 1.20859908 | 6.39798409 | 4.25895039 | 0.00012712 | 0.04474124 | 1.06341818 |

12 hr:

| | | | | | | | |
|---|---|---|---|---|---|---|---|
| FBXO45 | 225099_at | 1.13350791 | 9.69247626 | 5.51958363 | 2.40E−06 | 0.03282196 | 3.99779146 |
| ZNF566 | 240239_at | 1.20902406 | 6.20206745 | 5.35216774 | 4.09E−06 | 0.04477351 | 3.57707114 |

[symbol]

indicates data missing or illegible when filed

TABLE 4b
Probioglat expression values for key genes compared to the
range set by observed expression values under Copaxone ® treatment

| | | Copaxone | | | | | | | |
|---|---|---|---|---|---|---|---|---|---|
| | | Expression Value | | | | | | | |
| | | Range | | Probioglat Expression Values | | | | | |
| | | Maxi- | Mini- | Sample | Sample | Sample | Sample | Sample | Sample |
| Probeset | Gene | mum | mum | 1 | 2 | 3 | 4 | 5 | 6 |
| 203936_s_at | MMP9 | 9.97 | 9.53 | 10.14 | 10.05 | 10.12 | 10.14 | 10.04 | 10.04 |
| 201743_at | CD14 | 9.69 | 9.32 | 9.75 | 9.75 | 9.67 | 9.79 | 9.71 | 9.86 |
| 202637_s_at | ICAM1 | 8.06 | 7.44 | 8.14 | 7.96 | 8.26 | 7.84 | 8.18 | 7.92 |
| 202638_s_at | ICAM1 | 7.30 | 6.51 | 7.67 | 7.54 | 7.45 | 7.32 | 7.19 | 6.94 |
| 223961_s_at | CISH | 7.69 | 7.24 | 7.31 | 7.20 | 7.41 | 7.37 | 7.62 | 7.23 |
| 223377_x_at | CISH | 9.11 | 8.86 | 8.81 | 8.81 | 8.82 | 8.85 | 8.84 | 8.88 |

Red text indicates that the expression value for this Probioglat treated sample is outside of the observed range of all Copaxone samples.

### Discussion

### Example 2

### Pro-Inflammatory Genes

Gene-level differentiation analysis identifies specific pro-inflammatory markers when comparing Probioglat to Copaxone®. Upon comparison of GA with Probioglat, significant gene-expression differences were seen (Table 3). Only one batch of Probioglat was available to compare to the four batches of GA, prohibiting the possibility to study batch-to-batch variability. However, the range of variation defined by the four GA batches represents a range of variation that has been demonstrated to be safe and effective by Copaxone®'s clinical trials. The fact that any single batch of Probioglat results in values outside of that range (as illustrated in Table 4b and FIG. 44), coupled with lack of PK or PD markers to determine equivalence of the two glatiramoids, warrants further investigation.

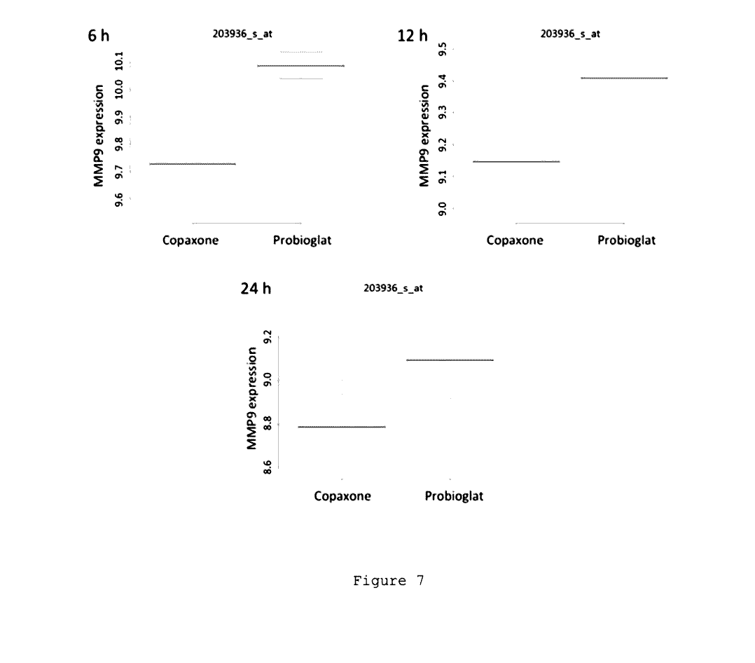

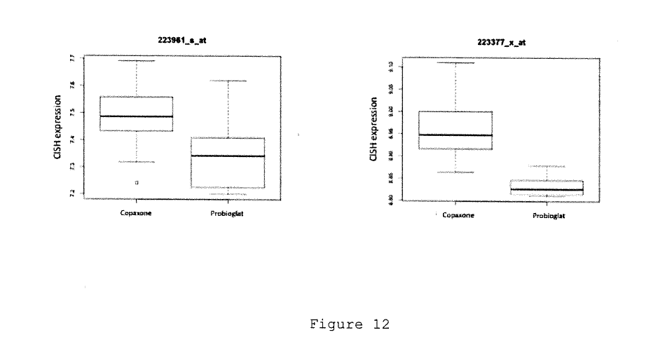

The consistent confirmatory results obtained by single-probeset, pathway and independent qRT-PCR analysis are particularly robust, given the stringent statistical framework employed. It should be noted that fewer genes were significantly modulated by GA relative to Probioglat than by GA relative to mannitol, an observation expected given the intended mimicry of structure between the compounds. Indeed many genes were modulated in the same direction by both GA and Probioglat versus control, but to differing extents (the cases for many genes discussed below, except where noted). It is striking that differences were observed between branded GA and the designed purported generic, Probioglat. Two drugs cannot be said to have identical effects if significant differences are manifest. Importantly, the significant differences in gene expression observed between Probioglat and Copaxone® were seen in genes tied to highly relevant disease pathoetiology and known GA mode of action (MOA). Bioinformatic analysis of differentially expressed genes (by FDR corrected p value) following Probioglat versus Copaxone® stimulation of human monocytes at 6 hours identified a number of genes tied to important immune system functions, in particular inflammation: CCL5, CCL2, MMP9, MMP1, CXCL10, CD14, ICAM1 and BIRC3. Several of these genes have been reported in the literature as modulated by GA treatment in patients.
1. CCL5 (RANTES) is a key chemokine thought to attract inflammatory immune cells to the CNS, and was significantly upregulated in Probioglat treatment relative to Copaxone® treatment at 6 hours (FDR adjusted p value 0.018, FC 1.09 in gene expression analysis; p value 4e-5, FC 1.12 in qRT-PCR confirmation). Indeed, an antibody blocking CCL5 was shown to reduce disease metrics including immune infiltration into the CNS in a viral MS model (Glass et al, Immunol Res, 2004; Chirstensen et al. Mult. Scler. J. 2013). Expression of the CCL5 receptor, CCR5, on GA-reactive T cells from MS patients was shown to be downregulated by chronic (1 year) GA treatment (Allie et al, Arch Neurol 2005; Kouwenhoven et al. J. Neuroimmunol. 2002). This gene was tested and confirmed to be upregulated with Probioglat treatment relative to GA treatment in primary human monocytes (p<0.029, FC 1.53).
2. Expression of MMP9 (Matrix Metalloproteinase 9) was significantly higher with Probioglat versus GA stimulation at 6h (FDR-adjusted p-value 2.8e-6, FC 1.29 in gene-expression analysis, FIG. 41a; p-value 0.02, FC 1.24 in qRT-PCR confirmation), and at 24h (FDR-adjusted p-value 0.004, FC 1.25). The MMP9 gene was also upregulated with Probioglat relative to GA treatment in primary human monocytes (p<0.009, FC 1.4). This protein is reported to increase access of immune cells to the CNS by contributing to disruption of the blood brain barrier (BBB) (Rosenberg et al, The Lancet 2005), and high levels of MMP9 have been associated with Multiple Sclerosis (Christensen et al, Mult. Scler 2013; Milward et al., J. Neuroimmunol. 2008; Knop et al, Neurology 2013; Parks et al, Nat. Rev. Immunol 2004). MMP9 was upregulated with Probioglat relative to Copaxone® stimulation at 6 hours (FDR adjusted p value 2.8e-6 in gene expression analysis; p value 0.02 in qRT-PCR confirmation). Expression levels of MMP9 at both the mRNA and protein level were increased in immature dendritic cells from MS patients relative to healthy controls (Kouwenhoven et al, J Neuroimmunol 2002). Elevated MMP9 levels were reported in patients with gadolinium-enhancing lesions relative to patients without (Waubant et al, Dis Markers 2006; Antonelli et al, Autoimmun. Rev. 2014), and MMP9 has been proposed as a biomarker for both MS diagnosis and progression (Milward et al, J Neuroimmunol 2008; Peperzak et al, J. Immunol. Batim. Md 1950, 2013). GA has been reported to inhibit MMP9 expression in healthy human peripheral blood mononuclear cells (PBMC) (Knop et al, Neurology 2013 (Meeting Abstract); Adrem and Ulevitch, Nature 2000). Increased expression of MMP9 for Probioglat relative to Copaxone® at multiple timepoints (FIG. 7).
3. The level of MMP1, another matrix metalloproteinase gene, was increased after Probioglat stimulation compared to Copaxone® at 6 hours (FDR adjusted p value 0.002, FC 1.50 in gene expression analysis; p value 0.02, FC 1.25 in qRT-PCR confirmation). Matrix metalloproteinases are known to cleave pro-inflammatory cytokines and chemokines to regulate inflammation (Parks et al, Nat Rev Immunol, 2004; Baumann et al, J. Exp. Med. 2010). Levels of MMP1 mRNA, as well as secreted MMP1, were observed to be higher in immature dendritic cells from MS patients relative to healthy controls (Kouwenhoven et al, J Neuroimmunol 2002; Parks et al, Nat Rev Immunol, 2004).
4. Expression of the chemokine CXCL10 was increased by Probioglat stimulation compared to Copaxone® at 6 hours (FDR adjusted p value 0.0006, FC 1.46 in gene expression analysis; p value 0.0029, FC 2.28 in qRT-PCR confirmation). This finding was confirmed by qRT-PCR in primary human monocytes, where CXCL10 was upregulated by Probioglat relative to Copaxone treatment with p value<0.02 and fold change of 2.1. CXCL10 level in peripheral fluids was previously shown as associated with host immune response, particularly with regard to Th-1 cells (Antonelli et al, Autoimm. Rev., 2014; Natarajan et al, J. Nueroimmunol. 2013). CXCL10 is involved in recruiting CD8+ and Th1 CD4+ effector T cells to sites of inflammation (Peperzak et al, J Immunol, 2013; Hedegaard et al, PLoS ONE 2011). A study using monocytes from RRMS patients demonstrated CXCL10 to be increased by GA therapy within the first two months of treatment (Thamilarasan et al, J Neuroinflammation 2013; Rosenberg, The Lancet 2005).
5. CD14 is a marker of monocyte activation known to enhance inflammatory responses. CD14 was upregulated in human monocytes stimulated by Probioglat versus Copaxone 6 hours (FDR adjusted p value 0.006, FC 1.17; qRT-PCR not tested; FIG. 41b). CD14 was not modulated by GA treatment versus mannitol control. This marker of monocyte activation enhances inflammatory responses (Mycko et al, Ann. Neurol. 1998). In complex with LPS binding protein (LBP), CD14 interacts with LPS and helps to present it to toll-like receptor 4 (TLR4), activating downstream expression of inflammatory genes via NF-kB (Park et al, Exp Mol Med, 2013). CD14 has also been shown to be a coreceptor for other TLRs, and was demonstrated to be required for induction of proinflammatory cytokines via TLR7 and TLR9 in mouse and human cells in vitro (Baumann et al, J Exp Med, 2010; Bullard et al, J. Immunol. 2007). Increased expression of CD14 for Probioglat was determined relative to Copaxone® (FIG. 8).
6. CARD15 (NOD2), another gene upregulated by Probioglat versus Copaxone at 6 hours (FDR adjusted p value 0.02, FC 1.14; qRT-PCR not tested), is also a key player in the immune response to LPS, participating in activation of NF-kB. Activation of NOD2 by peptidoglycan has been shown to induce CNS demyelination in rats (Natarajan et al, 2013; Sellner et al, Clin. Exp. Immunol. 2013). In addition, a SNP in NOD2 was shown to affect the responses of Th2 and Th17 cells to myelin basic protein (MBP) in MS (Hedegaard et al, 2011; Bertrand et al, Immunity 2009).
7. BIRC3 expression was increased by Probioglat relative to Copaxone® at 6 hours (FDR adjusted p value 0.018, FC 1.26; qRT-PCR not tested). This gene codes for an Inhibitor of Apoptosis Protein (IAP-1), which in addition to its role in cell survival plays a role in both innate immunity (Bertrand, 2009; Kearny et al, J. Biol. Chem. 2013) and inflammation (Labbe et al, 2011; Ashburner et al, Nat. Genet. 2000), and has been suggested to have an immunomodulatory effect in autoimmune demyelination (Hebb et al, 2008; Kanehisa et al, Nucleic Acis Res. 2000). IAPs including IAP-1 are required for production of pro-inflammatory cytokines via several different pathways, including TLR4 activation (Tseng et al, 2010; Zanin-Zhorov et al, J. Clin. Invest. 2006) and NOD2 activation by TNFα (Kearney et al, JBC, 2012; Qin et al, Proc. Natl. Acad. Sci. 2012).
   CCL2 (MCP-1) is another pro-inflammatory cytokine that was also expressed significantly more highly with Probioglat treatment relative to Copaxone at 6 hours (FDR adjusted p value 0.003, FC 1.25; qRT-PCR not tested). CCL2 expression was decreased by GA treatment relative to mannitol control, and decreased to a lesser extent by Probioglat relative to mannitol control. CCL2 is thought to recruit inflammatory cells into the CNS in EAE and in MS (Mahad et al, 2006; Waubant, Dis. Markers 2006). This gene was also confirmed to be upregulated with Probioglat relative to GA treatment in primary human monocytes (p<0.009, FC 1.24).
8. ICAM1 expression was increased by Probioglat relative to Copaxone® at 6 hours (FDR adjusted p value 0.004, FC 1.41; qRT-PCR not tested; FIG. 41c). ICAM1 is an adhesion molecule that plays a key role in inflammatory processes by promoting leukocyte adhesion to the endothelium of the vascular wall, and is known to have an important role in inflammatory cell infiltration into the CNS in both EAE and MS (Mycko et al, Ann. Neurol. 1994; Labbe et al, Immunity 2011). In mice null for ICAM1, T cells produced significantly less IFNγ and showed much less infiltration into the spinal cord (Bullard et al, J Immunol, 2007; Hebb et al, Mult. Scler. Houndmills Basingstoke Engl. 2008). In PBMC from RRMS patients, ICAM1 levels were higher versus healthy controls, and chronic treatment with GA affected surface ICAM1 levels in multiple immune cell types (Sellner et al, Clin Exp Immunol 2013; Tseng et al, Nat. Immunol. 2010). Increased expression of ICAM1 for Probioglat was determined compared to Copaxone® stimulation (FIG. 11).

### Anti-Inflammatory Genes

As discussed herein, a number of pro-inflammatory genes and pathways were shown to be significantly upregulated by Probioglat as compared to Copaxone®. At the same time, several anti-inflammatory genes were downregulated by Probioglat stimulation in comparison with Copaxone® at 6 hours.
1. CISH, also known as SOCS (Suppressor of Cytokine Signaling), was expressed at lower level with Probioglat relative to Copaxone® at 6 hours (FDR adjusted p value 0.03, FC −1.09; FIG. 41d). A closely related protein, SOCS3, has been shown in myeloid cells to protect from EAE, the mouse model of MS, via deactivating neuroinflammatory responses (Qin et al, PNAS 2012; Bakshi et al, Expert Opin. THer. Targets 2013). Both present probesets for this gene were affected in the same direction (FIG. 12). A SNP in SOCS1, another family member, has been identified as a risk factor for MS (Towfic et al, PLoS ONE 2014).
2. HSPD1, also known as HSP-60 (heat shock 60 kDa protein 1), was downregulated in Probioglat relative to Copaxone® at 6 hours (FDR adjusted p value 0.01, FC −1.31). Zanin-Zhorov et al (2006) showed that HSP60 as well as synthetic peptide derived from HSP60 act as co-stimulators of Tregs through the TLR2 pathway. Tregs are immune regulatory cells that inhibit lympho-proliferation and IFNG and TNF secretion by pro-inflammatory T cells. Zanin-Zhorov et al concluded that HSP60 can downregulate adaptive immune responses by upregulating Tregs. Thus, downregulation of HSPD1 may result in less inhibition of immune response by Probioglat compared to Copaxone®.

It should be noted, however, the anti-inflammatory cytokine IL10, known to be relevant to the GA mechanism of action, was also expressed at a higher level subsequent Probioglat relative to Copaxone treatment at 6 hours (FDR adjusted p value 0.005, FC 1.28). The same observation holds for another gene at 6 and 12 hours, PRDM1 (Blimp1) (FDR adjusted p value 0.0006 and 7.7e-6, and FC 1.31 and 1.31 respectively), that when deleted results in inflammatory pathology (Chiang et al, PNAS 2013; Johnson et al, Biostat. Oxf. Engl. 2007). Blimp1 is a target of FOXP3 and is needed for production of IL10 by Tregs; its expression can also be induced by IL2 and proinflammatory cytokines in Tregs (Cretney et al, Nat Immunol. 2011; Leek et al, Surrogate Variabel Analysis 2013). However, it is not clear what these observations would imply for APCs such as monocytes. It is possible that higher Blimp1 could be an attempted protective response to a higher inflammatory milieu. A statidtically significant difference in such a mechanistically relevant gene—in either direction—between two therapeutics intended to be identical presents motivation for further study.

### Example 3

### Pathway Enrichment Analysis

Upregulated and downregulated genes were analyzed separately for pathway enrichment, using DAVID (Huang et al, Nucleic Acids Res 2009).

Pathway enrichment results were visualized using volcano plots, plotting −log p values versus enrichment scores. For GA MOA, to obtain top-gene lists of appropriate size (tens-hundreds) for use with DAVID, an absolute-value-fold-change cutoff of 1.5 and p-value cutoff of 1e-5 were utilized to obtain gene lists for pathway enrichment at 6h. For comparisons between branded GA and Probioglat, upregulated or downregulated genes with FDR-adjusted p values<0.05 were used for pathway enrichment.

DAVID runs were conducted May 21, 2014. Please note that the GO databases are updated daily (as noted on the GO site:www.geneontology.org/GO.downloads.ontology.shtml) and therefore performing the same enrichments on the same genesets may yield slightly varying results depending on the run date, as illustrated by the differences in Table 6 (results from runs on differing dates using broader or more restrictive subsets of GO). Thus, the pathway p values may change slightly between runs conducted at different times; the overall picture of enriched pathways, however, is expected to remain consistent. Three time points were tested to identify the fold change and p value filters were used to obtain top gene lists of appropriate size (i.e. tens to hundreds) for use with DAVID (Table 5).

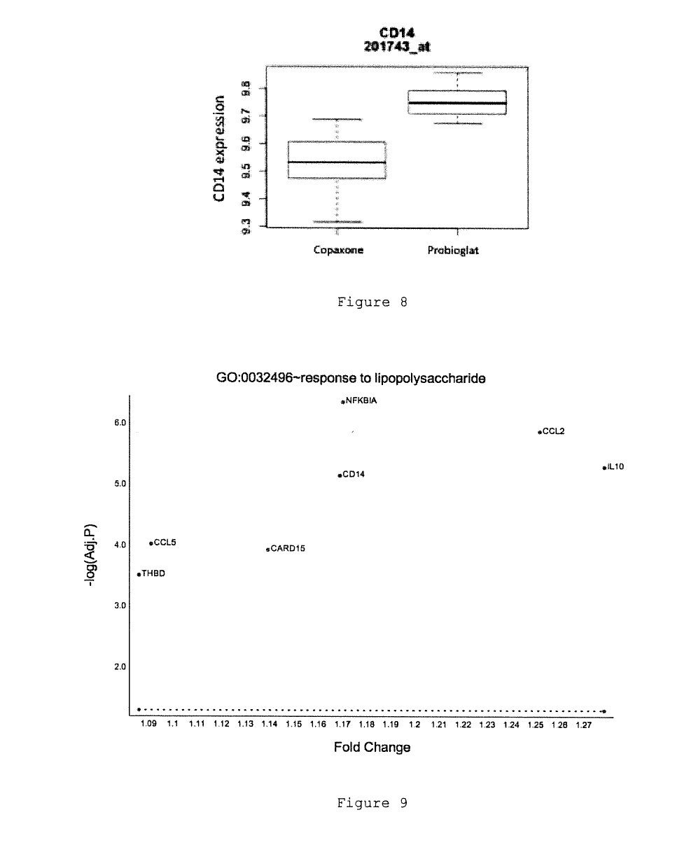

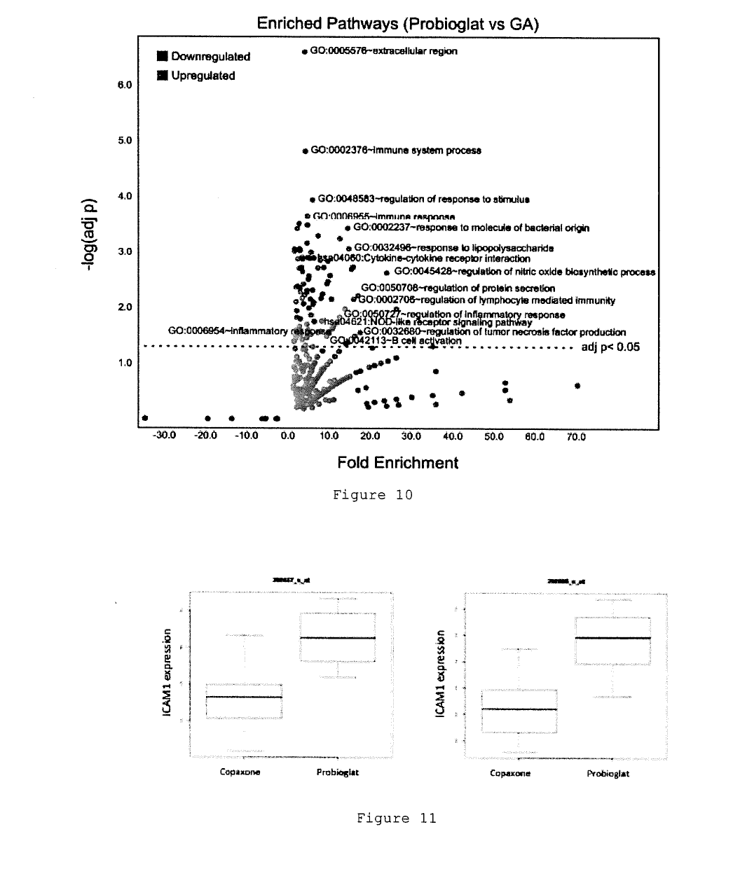

No pathways were enriched significantly among downregulated genes, however 106 pathways were enriched significantly (Benjamini corrected p value<0.05) among genes upregulated by Probioglat relative to GA, including immune system process (GO:0002376), immune system process (GO:0002376) and immune response (GO:0006955) pathways (Benjamini-corrected p-values 1.5e-5 and 3.3e-4, respectively), and many other immune system related pathways, such as regulation of lymphocyte mediated immunity (GO:0002706, Benjamini-corrected p-value 0.007) and B cell proliferation (GO:0042100 Benjamini-corrected p-value 0.049) (FIG. 10 and Table 6). Multiple significantly enriched pathways were relevant to inflammation, including response to lipopolysaccharide (LPS) (GO:0032496; FIG. 9), regulation of inflammatory response (GO:0050727), regulation of tumor necrosis factor production (GO:0032680), and NOD-like receptor signaling (hsa04621) (Benjamini-corrected p-values of 8.7e-4, 0.015, 0.028, and 0.027, respectively). No pathways were enriched significantly among genes downregulated by Probioglat versus GA.

Genes analyzed for pathway enrichment (Tables 5 and 6), using DAVID (conducted May 21, 2014). Performing the same enrichments on the same genesets may yield slightly varying results depending on the run date (GO databases are updated daily: www.geneontology.org/GO.downloads.ontology.shtml).

TABLE 5
Human monocyte study: pathways significantly enriched among
genes significantly upregulated by Copaxone ® relative to mannitol at
6 hours.
Pathways significantly enriched among top genes modulated by GA relative to mannitol control at 6 hours:
Top genes are defined by cutoffs of |FC|>1.5 and p-value<1e−5 to obtain lists of appropriate size for use with DAVID.

| | | | |
|---|---|---|---|
| Term | P value | Fold Enrichment | Benjamini |

Enriched among top downregulated genes:

| | | | |
|---|---|---|---|
| GO: 0007275~multicellular | 6.94E−06 | 2.168367347 | 0.00270611 |
| organismal | | | |
| development | | | |
| GO: 0048731~system | 5.04E−06 | 2.396166134 | 0.00294797 |
| development | | | |
| GO: 0048513~organ | 2.61E−06 | 2.760084926 | 0.00305609 |
| development | | | |
| GO: 0048856~anatomical | 3.69E−05 | 2.164502165 | 0.00861537 |
| structure development | | | |
| GO: 0030154~cell | 4.49E−05 | 2.555555556 | 0.00871888 |
| differentiation | | | |
| GO: 0032502~developmental | 3.28E−05 | 1.984126984 | 0.00955745 |
| process | | | |
| GO: 0003006~reproductive | 8.34E−05 | 6.25 | 0.01385786 |
| developmental | | | |
| process | | | |
| GO: 0048869~cellular | 0.00010797 | 2.410901468 | 0.0156803 |
| developmental process | | | |
| GO: 0009653~anatomical | 0.00030745 | 2.643171806 | 0.03921862 |
| structure morphogenesis | | | |

Enriched among top upregulated genes:

| | | | |
|---|---|---|---|
| GO: 0005886~plasma | 1.02E−13 | 2.419799407 | 2.18E−17 |
| membrane | | | |
| GO: 0031226~intrinsic to | 1.09E−11 | 3.267558528 | 7.78E−10 |
| plasma membrane | | | |
| GO: 0016020~membrane | 8.59E−12 | 1.538151311 | 9.23E−10 |
| GO: 0005887~integral to | 2.42E−11 | 3.249703017 | 1.04E−09 |
| plasma membrane | | | |
| GO: 0044459~plasma | 1.97E−11 | 2.403721216 | 1.06E−09 |
| membrane part | | | |
| GO: 0005576~extracellular | 1.37E−10 | 2.823873071 | 4.90E−Q9 |
| region | | | |
| GO: 0031224~intrinsic to | 4.97E−10 | 1.683683794 | 1.53E−08 |
| membrane | | | |
| GO: 0004871~signal | 1.04E−10 | 2.537224567 | 2.51E−08 |
| transducer activity | | | |
| GO: 0060089~molecular | 1.04E−10 | 2.537224567 | 2.51E−08 |
| transducer activity | | | |
| GO: 0004872~receptor | 6.20E−11 | 2.976306146 | 2.99E−08 |
| activity | | | |
| GO: 0044425~membrane | 1.40E−09 | 1.538980298 | 3.75E−08 |
| part | | | |
| GO: 0016021~integral to | 1.98E−09 | 1.675513671 | 4.73E−08 |
| membrane | | | |
| GO: 0044421~extracellular | 3.24E−09 | 3.610050499 | 6.97E−08 |
| region part | | | |
| GO: 0004888~transmembrane | 2.35E−09 | 3.768030976 | 3.78E−07 |
| receptor activity | | | |
| GO: 0006955~immune | 9.81E−10 | 3.570488309 | 6.34E−07 |
| response | | | |
| GO: 0009611~response | 3.29E−10 | 4.097150568 | 6.39E−07 |
| to wounding | | | |
| GO: 0009605~response | 7.26E−10 | 3.127282815 | 7.04E−07 |
| to external stimulus | | | |
| GO: 0002376~immune | 1.92E−09 | 2.852452609 | 9.30E−07 |
| system process | | | |
| GO: 0050896~response | 2.41E−09 | 1.852190811 | 9.36E−07 |
| to stimulus | | | |
| GO: 00322501~multicellular | 7.40E−09 | 1.769727444 | 2.39E−06 |
| organismal process | | | |
| GO: 0050793~regulation | 3.28E−08 | 3.160728606 | 9.08E−06 |
| of developmental | | | |
| process | | | |
| GO: 0009897~external | 5.99E−07 | 6.496675192 | 1.17E−05 |
| side of plasma | | | |
| membrane | | | |
| GO: 0051239~regulation | 7.17E−08 | 2.791593852 | 1.54E−05 |
| of multicellular | | | |
| organismal process | | | |
| GO: 0006952~defense | 6.90E−08 | 3.417964583 | 1.67E−05 |
| response | | | |
| GO: 0006954~inflammatory | 8.72E−08 | 4.475172847 | 1.69E−05 |
| response | | | |
| GO: 0031012~extracellular | 1.51E−06 | 5.017117426 | 2.70E−05 |
| matrix | | | |
| GO: 0048583~regulation | 1.68E−07 | 3.612684989 | 2.96E−05 |
| of response to stimulus | | | |
| GO: 0005578~proteinaceous | 2.00E−06 | 5.309782609 | 3.07E−05 |
| extracellular matrix | | | |
| GO: 0009986~cell surface | 1.88E−06 | 4.06706753 | 3.10E−05 |
| GO: 0002682~regulation | 3.21E−07 | 3.763213531 | 5.19E−05 |
| of immune system | | | |
| process | | | |
| GO: 0005615~extracellular | 6.96E−06 | 3.371290545 | 9.98E−05 |
| space | | | |
| GO: 0048731~system | 7.36E−07 | 1.917675905 | 0.00010967 |
| development | | | |
| GO: 0051707~response | 1.25E−06 | 4.4259178 | 0.00017294 |
| to other organism | | | |
| GO: 0007275~multicellular | 2.71E−06 | 1.742406265 | 0.00032825 |
| organismal | | | |
| development | | | |
| GO: 0032101~regulation | 2.59E−06 | 6.287901089 | 0.00033427 |
| of response to external | | | |
| stimulus | | | |
| GO: 0022610~biological | 3.65E−06 | 2.940010571 | 0.00039282 |
| adhesion | | | |
| GO: 0007155~cell | 3.47E−06 | 2.948386669 | 0.00039594 |
| adhesion | | | |
| GO: 0048856~anatomical | 4.11E−06 | 1.792006443 | 0.00041352 |
| structure development | | | |
| GO: 0065008~regulation | 5.75E−06 | 2.067363531 | 0.00050661 |
| of biological quality | | | |
| GO: 0007166~cell surface | 5.31E−06 | 2.24578872 | 0.00051425 |
| receptor linked signal | | | |
| transduction | | | |
| GO: 0050727~regulation | 5.72E−06 | 8.870431894 | 0.00052809 |
| of inflammatory | | | |
| response | | | |
| GO: 0006950~response | 6.47E−06 | 1.896821914 | 0.0005449 |
| to stress | | | |
| GO: 0032502~developmental | 9.45E−06 | 1.642672573 | 0.00073237 |
| process | | | |
| GO: 0031347~regulation | 9.45E−06 | 5.519379845 | 0.0007628 |
| of defense response | | | |
| GO: 0009607~response | 1.20E−05 | 3.375586386 | 0.00089042 |
| to biotic stimulus | | | |
| GO: 0019955~cytokine | 1.09E−05 | 6.970960427 | 0.00131926 |
| binding | | | |
| GO: 0050776~regulation | 2.06E−05 | 4.292850991 | 0.00147588 |
| of immune response | | | |
| GO: 0040011~locomotion | 2.38E−05 | 3.210251542 | 0.00164377 |
| GO: 0080134~regulation | 2.54E−05 | 3.700142913 | 0.00169407 |
| of response to stress | | | |
| GO: 0007626~locomotory | 3.53E−05 | 4.081231576 | 0.00227722 |
| behavior | | | |
| GO: 0002684~positive | 4.09E−05 | 4.024547804 | 0.00255401 |
| regulation of immune | | | |
| system process | | | |
| GO: 0000267~cell | 0.00027348 | 1.99311488 | 0.00366864 |
| fraction | | | |
| GO: 0045028~purinergic | 5.12E−05 | 13.86854232 | 0.00411604 |
| nudeotide receptor | | | |
| activity, G-protein | | | |
| coupled | | | |
| GO: 0001608~nucleotide | 5.12E−05 | 13.86854232 | 0.00411604 |
| receptor activity, G- | | | |
| protein coupled | | | |
| GO: 0004930~G-protein | 4.66E−05 | 4.292644053 | 0.00449193 |
| coupled receptor activity | | | |
| GO: 0016502~nucleotide | 8.64E−05 | 12.54772877 | 0.00594437 |
| receptor activity | | | |
| GO: 0001614~purinergic | 8.64E−05 | 12.54772877 | 0.00594437 |
| nucleotide receptor | | | |
| GO: 0005626~insoluble | 0.00050614 | 2.119836435 | 0.00638231 |
| friction | | | |
| GO: 0048513~organ | 0.00010948 | 1.845652496 | 0.00640896 |
| development | | | |
| GO: 0002697~regulation | 0.00010851 | 6.009002251 | 0.00655072 |
| of immune effector | | | |
| process | | | |
| GO: 0051384~response | 0.00011695 | 7.046016823 | 0.00664447 |
| to glucocorticoid | | | |
| stimulus | | | |
| GO: 0005624~membrane | 0.00064098 | 2.128163382 | 0.00762936 |
| fraction | | | |
| hsa04060:Cytokine- | 7.88E−05 | 3.910866911 | 0.00816277 |
| cytokine receptor | | | |
| interaction | | | |
| GO: 0031349~positive | 0.00015337 | 6.7584243 | 0.00822279 |
| regulation of defense | | | |
| response | | | |
| GO: 0048584~positive | 0.00015308 | 3.789715034 | 0.00844122 |
| regulation of response to | | | |
| stimulus | | | |
| GO: 0031960~response | 0.00017474 | 6.623255814 | 0.00911158 |
| to corticosteroid | | | |
| stimulus | | | |
| GO: 0042221~response | 0.00023157 | 1.879298045 | 0.01115792 |
| to chemical stimulus | | | |
| GO: 0065007~biological | 0.00022752 | 1.23581669 | 0.01124356 |
| regulation | | | |
| GO: 0050865~regulation | 0.00022283 | 4.295743747 | 0.01130118 |
| of cell activation | | | |
| GO: 0048545~response | 0.00025981 | 4.216192937 | 0.0122074 |
| to steroid hormone | | | |
| stimulus | | | |
| GO: 0006935~chemotaxis | 0.00027256 | 4.651162791 | 0.01249951 |
| GO: 0042330~taxis | 0.00027256 | 4.651162791 | 0.01249951 |
| GO: 0002703~regulation | 0.0003028 | 7.429934407 | 0.01355657 |
| of leukocyte mediated | | | |
| immunity | | | |
| GO: 0002822~regulation | 0.00034918 | 7.244186047 | 0.01492816 |
| of adaptive immune | | | |
| response based on | | | |
| somatic recombination | | | |
| of immune receptors | | | |
| built from | | | |
| immunoglobulin | | | |
| superfamily domains | | | |
| G0: 0001568~blood | 0.00034714 | 3.471867967 | 0.01517609 |
| vessel development | | | |
| G0: 0030154~cell | 0.00037713 | 1.79379845 | 0.01576601 |
| differentiation | | | |
| GO: 0001944~vasculature | 0.00039042 | 3.42764035 | 0.01597268 |
| development | | | |
| G0: 0002819~regulation | 0.00040094 | 7.067498582 | 0.0160609 |
| of adaptive immune | | | |
| response | | | |
| GO: 0051241~negative | 0.0004538 | 4.902080783 | 0.01743952 |
| regulation of | | | |
| multicellular organismal | | | |
| GO: 0051704~multi- | 0.00044925 | 2.222569065 | 0.01761538 |
| organism process | | | |
| GO: 0007165~signal | 0.00048801 | 1.566926487 | 0.01837798 |
| transduction | | | |
| GO: 0009617~response | 0.00051926 | 4.267561736 | 0.01917123 |
| to bacterium | | | |
| GO: 0048519~negative | 0.00057125 | 1.628669462 | 0.01993331 |
| regulation of biological | | | |
| process | | | |
| GO: 0050864~regulation | 0.00056871 | 8.564554932 | 0.0202092 |
| of B cell activation | | | |
| GO: 0001525~angiogenesis | 0.00056006 | 4.224015187 | 0.02027648 |
| GO: 0048869~cellular | 0.00059519 | 1.735654039 | 0.02039329 |
| developments process | | | |
| GO: 0002694~regulation | 0.00064956 | 4.139534884 | 0.02184996 |
| of leukocyte activation | | | |
| GO: 0044243~multicellular | 0.00070212 | 20.69767442 | 0.02319544 |
| organismal catabolic | | | |
| process | | | |
| GO: 0030574~collagen | 0.00070212 | 20.69767442 | 0.02319544 |
| cataboltc process | | | |
| GO: 0016477~cell | 0.00071879 | 3.203211517 | 0.02334233 |
| migration | | | |
| GO: 0045595~regulation | 0.0007682 | 2.517284727 | 0.02373537 |
| of cell differentiation | | | |
| GO: 0032879~regulation | 0.0007822 | 2.358709335 | 0.02378398 |
| of localization | | | |
| GO: 0050729~positive | 0.00076295 | 11.49870801 | 0.0239569 |
| regulation of | | | |
| inflammatory response | | | |
| GO: 0007186~G-protein | 0.00075328 | 2.725619677 | 0.0240464 |
| coupled receptor protein | | | |
| signaling pathway | | | |
| GO: 0051270~regulation | 0.00083518 | 3.642790698 | 0.02498336 |
| of cell motion | | | |
| GO: 0048518~positive | 0.0009722 | 1.559204424 | 0.02858412 |
| regulation of biological | | | |
| process | | | |
| GO: 001816~cytokine | 0.00105123 | 7.526427061 | 0.0299652 |
| production | | | |
| GO: 0007610~behavior | 0.00104771 | 2.759689922 | 0.03031177 |
| GO: 00550878~regulation | 0.00117703 | 4.87004104 | 0.03300809 |
| of body fluid levels | | | |
| GO: 0032496~response | 0.0013154 | 5.681714546 | 0.03578575 |
| to lipopolysaccharide | | | |
| GO: 0002683~negative | 0.0013154 | 5.681714546 | 0.03578575 |
| regulation of immune | | | |
| GO: 0051674~localization | 0.00131424 | 2.989664083 | 0.03626342 |
| of cell | | | |
| GO: 0048870~cell motility | 0.00131424 | 2.989664083 | 0.03626342 |
| GO: 0046903~secretion | 0.00139387 | 3.163975707 | 0.03684834 |
| GO: 0006928~cell motion | 0.0013811 | 2.469196246 | 0.03702169 |
| GO: 0048514~blood | 0.00142405 | 3.398125651 | 0.03712585 |
| vessel morphogenesis | | | |
| GO: 0001501~skeletal | 0.00146721 | 3.143950545 | 0.03772332 |
| system development | | | |
| GO: 0050867~positive | 0.00151961 | 4.664264658 | 0.03853451 |
| regulation of cell | | | |
| activation | | | |
| GO: 0030247~polysaccharide | 0.0007648 | 5.243826948 | 0.04514181 |
| binding | | | |
| GO: 0001871~pattern | 0.0007648 | 5.243826948 | 0.04514181 |
| binding | | | |
| GO: 0004222~metalloendopeptidase | 0.00087418 | 6.148387097 | 0.04585047 |
| activity | | | |
| GO: 0002698~negative | 0.00196103 | 15.05285412 | 0.0488232 |
| regulation of immune | | | |
| effector process | | | |
| GO: 0002698~positive | 0.00201705 | 6.536107711 | 0.04892898 |
| regulation of response to | | | |
| external stimulus | | | |
| GO: 0010033~response | 0.00199539 | 2.03004903 | 0.04902907 |
| to organic substance | | | |

| | |
|---|---|
| Term | Probesets |

Enriched among top downregulated genes:

| | |
|---|---|
| GO: 0007275~multicellular | 207865_S_AT, 41037_AT, 201350_AT, 229638_AT, 222162_S_AT, 204457_S_AT, |
| organismal | 39966_AT, 52651_AT, 227242_S_AT, 227236_AT, 200894_S_AT, 239410_AT, |
| development | 217028_AT, 206589_AT, 211299_S_AT, 208078_S_AT, 223704_S_AT, 201688_S_AT, |
| | 201689_S_AT, 216953_S_AT, 201690_S_AT, 229103_AT, 218326_S_AT, 202800_AT, |
| | 210347_S_AT, 232614_AT, 212558_AT, 206935_AT, 229084_AT, 219497_S_AT, |
| | 227103_S_AT, 235275_AT, 211919_S_AT, 212097_AT, 206067_S_AT, 243209_AT, |
| | 207725_AT, 209201_X_AT, 213478_AT, 213568_AT, 211421_S_AT, 221900_AT, |
| | 222891_S_AT, 203685_AT, 207717_S_AT |
| GO: 0048731~system | 207865_S_AT, 41037_AT, 201350_AT, 229638_AT, 222162_S_AT, 39966_AT, |
| development | 52651_AT, 227236_AT, 239410_AT, 217028_AT, 206589_AT, 211299_S_AT, |
| | 223704_S_AT, 201688_S_AT, 201689_S_AT, 216953_S_AT, 201690_S_AT, |
| | 229103_AT, 218326_S_AT, 202800_AT, 232614_AT, 210347_S_AT, 212558_AT, |
| | 206935_AT, 229084_AT, 219437_S_AT, 227103_S_AT, 235275_AT, 211919_S_AT, |
| | 206067_S_AT, 212097_AT, 243209_AT, 207725_AT, 209201_X_AT, 213478_AT, |
| | 213568_AT, 211421_S_AT, 221900_AT, 222891_S_AT, 203685_AT, 207717_S_AT |
| GO: 0048513~organ | 207865_S_AT, 41037_AT, 201350_AT, 222162_S_AT, 52651_AT, 239410_AT, |
| development | 217028_AT, 206589_AT, 211299_S_AT, 223704_S_AT, 201688_S_AT, 201689_S_AT, |
| | 216953_S_AT, 201690_S_AT, 229103_AT, 218326_S_AT, 232614_AT, 210347_S_AT, |
| | 212558_AT, 206935_AT, 229084_AT, 219497_S_AT, 227103_S_AT, 235275_AT, |
| | 211919_5_AT, 206067_S_AT, 212097_AT, 243209_AT, 207725_AT, 209201_X_AT, |
| | 213478_AT, 211421_S_AT, 213568_AT, 221900_AT, 203685_AT, 2228911_S_AT, |
| | 207717_S_AT |
| GO: 0048856~anatomical | 207865_S_AT, 41037_AT, 201350_AT, 229638_AT, 222162_S_AT, 39966_AT, |
| structure development | 52651_AT, 227236_AT, 239410_AT, 217028_AT, 206589_AT, 211299_S_AT, |
| | 223704_S_AT, 201688_S_AT, 201689_S_AT, 216953_S_AT, 201690_S_AT, |
| | 229103_AT, 218326_S_AT, 202800_AT, 232614_AT, 210347_S_AT, 212558_AT, |
| | 206935_AT, 229084_AT, 219497_S_AT, 227103_S_AT, 235275_AT, 211919_S_AT, |
| | 206067_S_AT, 212097_AT, 243203_AT, 207725_AT, 209201_X_AT, 213478_AT, |
| | 213568_AT, 211421_S_AT, 221900_AT, 222891_S_AT, 203685_AT, 207717_S_AT |
| GO: 0030154~cell | 207865_S_AT, 213361_AT, 41037_AT, 229638_AT, 204557_S_AT, 39966_AT, |
| differentiation | 227236_AT, 217028_AT, 206539_AT, 208078_S_AT, 201688_S_AT, 201688_S_AT, |
| | 216953_S_AT, 201690_S_AT, 229103_AT, 202800_AT, 210347_S_AT, 232614_AT, |
| | 212558_AT, 229084_AT, 219497_S_AT, 227103_S_AT, 235275_AT, 212097_AT, |
| | 206067_S_AT, 211919_S_AT, 207725_AT, 209201_X_AT, 213478_AT, 211421_S_AT, |
| | 222891_S_AT, 203685_AT |
| GO: 0032502~developmental | 207865_S_AT, 213361_AT, 41037_AT, 201350_AT, 229638_AT, 222162_S_AT, |
| process | 204557_S_AT, 39966_AT, 52651_AT, 227242_S_AT, 227236_AT, 200894_S_AT, |
| | 239410_AT, 217028_AT, 206589_AT, 211299_S_AT, 208078_S_AT, 223704_S_AT, |
| | 201688_S_AT, 201689_S_AT, 216953_S_AT, 201690_S_AT, 229103_AT, |
| | 218326_S_AT, 202800_AT, 210347_S_AT, 232614_AT, 212558_AT, 206935_AT, |
| | 229084_AT, 219497_S_AT, 227103_S_AT, 235275_AT, 211919_S_AT, 212097_AT, |
| | 206067_S_AT, 243209_AT, 207725_AT, 209201_X_AT, 213478_AT, 213568_AT, |
| | 211421_S_AT, 221900_AT, 222891_S_AT, 203685_AT, 207717_S_AT |
| GO: 0003006~reproductive | 213361_AT, 218326_S_AT, 232614_AT, 222162_S_AT, 204557_S_AT, 206067_S_AT, |
| developmental | 211919_S_AT, 200894_S_AT, 209201_X_AT, 217028_AT, 203685_AT, 223704_S_AT, |
| process | 216953_S_AT |
| GO: 0048869~cellular | 207865_S_AT, 213361_AT, 41037_AT, 229638_AT, 204557_S_AT, 39966_AT, |
| developmental process | 227236_AT, 217028_AT, 206589_AT, 208078_S_AT, 201688_S_AT, 201689_S_AT, |
| | 216953_S_AT, 201690_S_AT, 229103_AT, 202800_AT, 210347_S_AT, 232614_AT, |
| | 212558_AT, 229084_AT, 219497_S_AT, 227103_S_AT, 235275_AT, 212097_AT, |
| | 206067_S_AT, 211919_S_AT, 207725_AT, 209201_X_AT, 213478_AT, 211421_S_AT, |
| | 222891_S_AT, 203685_AT |
| GO: 0009653~anatomical | 41037_AT, 222162_S_AT, 526S1_AT, 217028_AT, 206589_AT, 201688_S_AT, |
| structure morphogenesis | 201689_S_AT, 216953_S_AT, 201690_S_AT, 229103_AT, 218326_S_AT, 202800_AT, |
| | 232614_AT, 229084_AT, 206935_AT, 206067_S_AT, 211919_S_AT, 212097_AT, |
| | 243209_AT, 207725_AT, 213568_AT, 211421_S_AT, 209201_X_AT, 221900_AT, |
| | 203685_AT |

Enriched among top upregulated genes:

| | |
|---|---|
| GO: 0005886~plasma | 219358_S_AT, 1553995_A_AT, 210495_X_AT, 229221_AT, 223723_AT, 212298_AT, |
| membrane | 209555_S_AT, 223660_AT, 211066_X_AT, 219412_AT, 205798_AT, 228579_AT, |
| | 230360_AT, 226545_AT, 242903_AT, 207610_S_AT, 220307_AT, 226629_AT, |
| | 201590_X_AT, 203922_S_AT, 229937_X_AT, 214830_AT, 203104_AT, 212977_AT, |
| | 202756_S_AT, 228766_AT, 230925_AT, 243556_AT, 201005_AT, 201242_S_AT, |
| | 242907_AT, 209122_AT, 205128_X_AT, 214211_AT, 237252_AT, 214297_AT, |
| | 202727_S_AT, 221060_S_AT, 211719_X_AT, 223596_AT, 223501_AT, 205898_AT, |
| | 34210_AT, 205099_S_AT, 218613_AT, 210146_X_AT, 215836_S_AT, 217678_AT, |
| | 232068_S_AT, 209047_AT, 238581_AT, 1554992_AT, 209906_AT, 243483_AT, |
| | 210510_S_AT, 211030_S_AT, 211676_S_AT, 208121_S_AT, 238669_AT, |
| | 204105_S_AT, 219994_AT, 201125_S_AT, 203355_S_AT, 202638_S_AT, |
| | 201243_S_AT, 204912_AT, 201069_AT, 206206_AT, 228918_AT, 222877_AT, |
| | 227134_AT, 203217_S_AT, 229900_AT, 244375_AT, 206488_S_AT, 206171_AT, |
| | 208789_AT, 1553151_AT, 220066_AT, 228640_AT, 237442_AT, 203939_AT, |
| | 205542_AT, 218589_AT, 243894_AT, 213428_S_AT, 205891_AT, 203665_AT, |
| | 207542_S_AT, 202748_AT, 208816_X_AT, 239519_AT, 208161_S_AT, 221266_S_AT, |
| | 209921_AT, 223502_S_AT, 1557938_S_AT, 212086_X_AT, 220484_AT, |
| | 203887_S_AT, 224341_X_AT, 223939_AT, 206637_AT, 1555756_A_AT, 209392_AT, |
| | 209993_AT, 215813_S_AT, 201324_AT, 212464_S_AT, 216442_X_AT, 213503_X_AT, |
| | 227240_AT, 210427_X_AT, 202609_AT, 214724_AT, 226218_AT, 204661_AT, |
| | 211661_X_AT, 210895_S_AT, 222062_AT, 204116_AT, 232746_AT, 223092_AT, |
| | 225188_AT, 202827_S_AT, 205419_AT, 235299_AT, 1560960_AT, 201952_AT, |
| | 225189_S_AT, 1552798_A_AT, 210264_AT, 205718_AT, 237904_AT, 203888_AT, |
| | 223798_AT, 212096_S_AT, 210258_AT, 210004_AT, 222876_S_AT, 229797_AT, |
| | 218223_S_AT, 241929_AT, 219434_AT, 201951_AT |
| GO: 0031226~intrinsic to | 223723_AT, 229221_AT, 209555_S_AT, 223660_AT, 203217_S_AT, 206488_S_AT, |
| plasma membrane | 206171_AT, 228579_AT, 228640_AT, 242903_AT, 205542_AT, 205891_AT, |
| | 207542_S_AT, 208161_S_AT, 203922_S_AT, 203104_AT, 202756_S_AT, |
| | 224341_X_AT, 203887_S_AT, 228766_AT, 209392_AT, 201005_AT, 201242_S_AT, |
| | 237252_AT, 214297_AT, 202727_S_AT, 221060_S_AT, 202609_AT, 223596_AT, |
| | 34210_AT, 204661_AT, 205898_AT, 205099_S_AT, 211661_X_AT, 222062_AT, |
| | 204116_AT, 210146_X_AT, 202827_S_AT, 223092_AT, 209047_AT, 205419_AT, |
| | 232068_S_AT, 209906_AT, 1560960_AT, 211030_S_AT, 210264_AT, 211676_S_AT, |
| | 208121_S_AT, 1552798_A_AT, 205718_AT, 237904_AT, 203888_AT, 204105_S_AT, |
| | 210004_AT, 241929_AT, 201125_S_AT, 202638_S_AT, 201243_S_AT |
| GO: 0016020~membrane | 1553995_A_AT, 223723_AT, 212298_AT, 223660_AT, 222838_AT, 219412_AT, |
| | 205798_AT, 205960_AT, 228579_AT, 238681_AT, 217997_AT, 226545_AT, |
| | 227747_AT, 205505_AT, 220307_AT, 226629_AT, 216080_S_AT, 201590_X_AT, |
| | 203922_S_AT, 218686_S_AT, 221815_AT, 203104_AT, 202756_S_AT, 228766_AT, |
| | 205128_X_AT, 214211_AT, 214581_X_AT, 63825_AT, 211719_X_AT, 223501_AT, |
| | 205898_AT, 205099_S_AT, 219574_AT, 210146_X_AT, 215836_S_AT, 235944_AT, |
| | 209047_AT, 240076_AT, 1554992_AT, 219926_AT, 235458_AT, 211676_S_AT, |
| | 225842_AT, 238669_AT, 204257_AT, 204105_S_AT, 224989_AT, 206134_AT, |
| | 202638_S_AT, 239761_AT, 201243_S_AT, 204312_AT, 238638_AT, 228318_AT, |
| | 227134_AT, 222877_AT, 212062_AT, 202434_S_AT, 222858_S_AT, 204222_S_AT, |
| | 229900_AT, 241392_AT, 206488_S_AT, 208789_AT, 1553151_AT, 220066_AT, |
| | 228640_AT, 205542_AT, 213338_AT, 203665_AT, 205891_AT, 202748_AT, |
| | 208816_X_AT, 239519_AT, 208161_S_AT, 218856_AT, 210145_AT, 223502_S_AT, |
| | 227889_AT, 236345_AT, 1557938_S_AT, 205003_AT, 212086_X_AT, 223939_AT, |
| | 203887_S_AT, 206637_AT, 211138_S_AT, 1555756_A_AT, 215813_S_AT, |
| | 227240_AT, 210427_X_AT, 226218_AT, 204661_AT, 225809_AT, 210895_S_AT, |
| | 222062_AT, 232746_AT, 210512_S_AT, 225188_AT, 211527_X_AT, 224990_AT, |
| | 1560960_AT, 201952_AT, 225189_S_AT, 210264_AT, 205718_AT, 210258_AT, |
| | 210757_X_AT, 222876_S_AT, 210004_AT, 229797_AT, 201280_S_AT, 201951_AT, |
| | 220333_AT, 219358_S_AT, 210495_X_AT, 229221_AT, 209555_S_AT, 202435_S_AT, |
| | 211066_X_AT, 217999_S_AT, 224480_S_AT, 242871_AT, 214255_AT, 230360_AT, |
| | 242903_AT, 228490_AT, 207610_S_AT, 229937_X_AT, 214830_AT, 227052_AT, |
| | 212977_AT, 230925_AT, 243556_AT, 201005_AT, 201242_S_AT, 242907_AT, |
| | 209122_AT, 237252_AT, 212171_X_AT, 214297_AT, 237030_AT, 202727_S_AT, |
| | 221060_S_AT, 223596_AT, 34210_AT, 205566_AT, 218613_AT, 217678_AT, |
| | 232068_S_AT, 238581_AT, 57715_AT, 209906_AT, 243483_AT, 210510_S_AT, |
| | 210513_S_AT, 211030_S_AT, 208121_S_AT, 223434_AT, 204881_S_AT, 225097_AT, |
| | 219994_AT, 205306_X_AT, 203355_S_AT, 201125_S_AT, 226136_AT, 201069_AT, |
| | 206206_AT, 203217_S_AT, 201278_AT, 244375_AT, 206171_AT, 218854_AT, |
| | 237442_AT, 203939_AT, 218589_AT, 243894_AT, 213428_S_AT, 202436_S_AT, |
| | 207542_S_AT, 221565_S_AT, 201279_S_AT, 219386_S_AT, 221266_S_AT, |
| | 209921_AT, 225337_AT, 220484_AT, 224341_X_AT, 228762_AT, 209392_AT, |
| | 201324_AT, 209993_AT, 212464_S_AT, 1555606_A_AT, 216442_X_AT, |
| | 213503_X_AT, 228708_AT, 202609_AT, 214724_AT, 211661_X_AT, 214841_AT, |
| | 204116_AT, 225207_AT, 219385_AT, 202827_S_AT, 223092_AT, 87100_AT, |
| | 205419_AT, 235286_AT, 235299_AT, 1554285_AT, 202437_S_AT, 1552798_A_AT, |
| | 203888_AT, 237904_AT, 223798_AT, 212096_S_AT, 218223_S_AT, 241929_AT, |
| | 219434_AT |
| GO: 0005887~integral to | 223723_AT, 229221_AT, 209555_S_AT, 223660_AT, 203217_S_AT, 206488_S_AT, |
| plasma membrane | 206171_AT, 228579_AT, 228640_AT, 242903_AT, 205542_AT, 205891_AT, |
| | 207542_S_AT, 208161_S_AT, 203922_S_AT, 203104_AT, 202756_S_AT, |
| | 224341_X_AT, 203887_S_AT, 228766_AT, 209392_AT, 201005_AT, 201242_S_AT, |
| | 237252_AT, 214297_AT, 202727_S_AT, 221060_S_AT, 202609_AT, 223596_AT, |
| | 34210_AT, 204661_AT, 205898_AT, 205099_S_AT, 211661_X_AT, 222062_AT, |
| | 204116_AT, 210146_X_AT, 202827_S_AT, 223092_AT, 209047_AT, 205419_AT, |
| | 232068_S_AT, 209906_AT, 211030_S_AT, 210264_AT, 211676_S_AT, 208121_S_AT, |
| | 1552798_A_AT, 205718_AT, 237904_AT, 203888_AT, 204105_S_AT, 210004_AT, |
| | 241929_AT, 201125_S_AT, 202638_S_AT, 201243_S_AT |
| GO: 0044459~plasma | 210495_X_AT, 223723_AT, 229221_AT, 209555_S_AT, 223660_AT, 227134_AT, |
| membrane part | 203217_S_AT, 219412_AT, 205798_AT, 206488_S_AT, 244375_AT, 206171_AT, |
| | 228579_AT, 208789_AT, 1553151_AT, 228640_AT, 237442_AT, 242903_AT, |
| | 205542_AT, 203665_AT, 205891_AT, 207542_S_AT, 202748_AT, 220307_AT, |
| | 208161_S_AT, 203922_S_AT, 214830_AT, 1557938_S_AT, 203104_AT, |
| | 202756_S_AT, 224341_X_AT, 203887_S_AT, 228766_AT, 1555756_A_AT, |
| | 230925_AT, 209332_AT, 201005_AT, 201242_S_AT, 209993_AT, 242907_AT, |
| | 212464_S_AT, 237252_AT, 216442_X_AT, 214297_AT, 202727_S_AT, 221060_S_AT, |
| | 211719_X_AT, 202609_AT, 214724_AT, 223596_AT, 226218_AT, 34210_AT, |
| | 204661_AT, 205898_AT, 205099_S_AT, 211661_X_AT, 210895_S_AT, 218613_AT, |
| | 222062_AT, 204116_AT, 210146_X_AT, 202827_S_AT, 223092_AT, 225188_AT, |
| | 209047_AT, 205419_AT, 232068_S_AT, 238581_AT, 209906_AT, 1560960_AT, |
| | 243483_AT, 201952_AT, 225189_S_AT, 211030_S_AT, 210264_AT, 211676_S_AT, |
| | 208121_S_AT, 1552798_A_AT, 205718_AT, 237904_AT, 203888_AT, 204105_S_AT, |
| | 210004_AT, 219994_AT, 241929_AT, 203355_S_AT, 201125_S_AT, 201951_AT, |
| | 202638_S_AT, 201243_S_AT |
| GO: 0005576~extracellular | 226136_AT, 223221_AT, 223723_AT, 210495_X_AT, 201069_AT, 212298_AT, |
| region | 205798_AT, 204222_S_AT, 229900_AT, 203936_S_AT, 226545_AT, 37145_AT, |
| | 203665_AT, 213428_S_AT, 208816_X_AT, 239519_AT, 201590_X_AT, 212190_AT, |
| | 223502_S_AT, 206157_AT, 212143_S_AT, 202458_AT, 203887_S_AT, 202756_S_AT, |
| | 201422_AT, 204834_AT, 205495_S_AT, 228873_AT, 209392_AT, 228762_AT, |
| | 210095_S_AT, 213265_AT, 209122_AT, 212464_S_AT, 237252_AT, 216442_X_AT, |
| | 237030_AT, 212171_X_AT, 213503_X_AT, 204575_S_AT, 1405_I_AT, 210427_X_AT, |
| | 211719_X_AT, 226218_AT, 223501_AT, 206835_AT, 207433_AT, 227265_AT, |
| | 235944_AT, 210512_S_AT, 202827_S_AT, 221463_AT, 211527_X_AT, 204475_AT, |
| | 235286_AT, 217757_AT, 229004_AT, 204655_AT, 210510_S_AT, 210513_S_AT, |
| | 203888_AT, 210004_AT, 206134_AT, 202087_S_AT, 1555759_A_AT, 1556423_AT, |
| | 219434_AT, 202638_S_AT |
| GO: 0031224~intrinsic to | 220333_AT, 1553995_A_AT, 229221_AT, 223723_AT, 212298_AT, 209555_S_AT, |
| membrane | 223660_AT, 222838_AT, 211066_X_AT, 205798_AT, 224480_S_AT, 214255_AT, |
| | 242871_AT, 238681_AT, 228579_AT, 230360_AT, 226545_AT, 242903_AT, |
| | 227747_AT, 228490_AT, 207610_S_AT, 205505_AT, 220307_AT, 226629_AT, |
| | 216080_S_AT, 203922_S_AT, 218686_S_AT, 229937_X_AT, 221815_AT, 214830_AT, |
| | 227052_AT, 203104_AT, 212977_AT, 202756_S_AT, 228766_AT, 201005_AT, |
| | 201242_S_AT, 214581_X_AT, 237252_AT, 237030_AT, 214297_AT, 63825_AT, |
| | 202727_S_AT, 221060_S_AT, 223596_AT, 223501_AT, 205898_AT, 34210_AT, |
| | 205099_S_AT, 205566_AT, 219574_AT, 210146_X_AT, 215836_S_AT, 217678_AT, |
| | 232068_S_AT, 240076_AT, 209047_AT, 57715_AT, 209906_AT, 243483_AT, |
| | 210510_S_AT, 211030_S_AT, 235458_AT, 219926_AT, 211676_S_AT, 208121_S_AT, |
| | 204257_AT, 223434_AT, 224989_AT, 204105_S_AT, 204881_S_AT, 206134_AT, |
| | 205306_X_AT, 201125_S_AT, 202638_S_AT, 239761_AT, 201243_S_AT, 226136_AT, |
| | 204912_AT, 238638_AT, 206206_AT, 228918_AT, 222877_AT, 203217_S_AT, |
| | 212062_AT, 229900_AT, 204222_S_AT, 206488_S_AT, 241392_AT, 206171_AT, |
| | 218854_AT, 228640_AT, 203939_AT, 205542_AT, 213318_AT, 218589_AT, |
| | 243894_AT, 205831_AT, 207542_S_AT, 221565_S_AT, 239519_AT, 219386_S_AT, |
| | 208161_S_AT, 221266_S_AT, 209921_AT, 218856_AT, 210145_AT, 227889_AT, |
| | 223502_S_AT, 225337_AT, 236345_AT, 220484_AT, 203887_S_AT, 224341_X_AT, |
| | 223939_AT, 206637_AT, 211138_S_AT, 1555756_A_AT, 209392_AT, 228762_AT, |
| | 209993_AT, 201324_AT, 1555606_A_AT, 228708_AT, 202609_AT, 226218_AT, |
| | 204661_AT, 225809_AT, 211661_X_AT, 214841_AT, 210895_S_AT, 222062_AT, |
| | 232746_AT, 204116_AT, 219385_AT, 223092_AT, 202827_S_AT, 205419_AT, |
| | 87100_AT, 235286_AT, 224990_AT, 235299_AT, 1560960_AT, 1554285_AT, |
| | 201952_AT, 1552798_A_AT, 210264_AT, 205718_AT, 237904_AT, 203888_AT, |
| | 223798_AT, 210004_AT, 229797_AT, 241929_AT, 219434_AT, 201951_AT |
| GO: 0004871~signal | 220333_AT, 204912_AT, 229221_AT, 212298_AT, 209555_S_AT, 223660_AT, |
| transducer activity | 206206_AT, 222838_AT, 222877_AT, 205798_AT, 206488_S_AT, 205960_AT, |
| | 206171_AT, 242871_AT, 200878_AT, 242903_AT, 218589_AT, 203760_S_AT, |
| | 203665_AT, 205891_AT, 207610_S_AT, 220307_AT, 239519_AT, 218856_AT, |
| | 218686_S_AT, 229937_X_AT, 203104_AT, 212977_AT, 223939_AT, 224341_X_AT, |
| | 203887_S_AT, 206637_AT, 228766_AT, 1555756_A_AT, 209332_AT, 214581_X_AT, |
| | 237252_AT, 214297_AT, 202727_S_AT, 1405_I_AT, 221060_S_AT, 202609_AT, |
| | 214724_AT, 226218_AT, 205898_AT, 205099_S_AT, 211661_X_AT, 210895_S_AT, |
| | 222062_AT, 204116_AT, 232746_AT, 225207_AT, 210146_X_AT, 235944_AT, |
| | 205419_AT, 232068_S_AT, 209906_AT, 243483_AT, 210510_S_AT, 204655_AT, |
| | 214054_AT, 211676_S_AT, 208121_S_AT, 1552798_A_AT, 210264_AT, |
| | 206675_S_AT, 205718_AT, 203888_AT, 237904_AT, 203761_AT, 210258_AT, |
| | 210004_AT, 1555759_A_AT, 241929_AT, 201125_S_AT, 219434_AT, 202638_S_AT |
| GO: 0060089~molecular | 220333_AT, 204912_AT, 229221_AT, 212298_AT, 209555_S_AT, 223660_AT, |
| transducer activity | 206206_AT, 222838_AT, 222877_AT, 205798_AT, 206488_S_AT, 205960_AT, |
| | 206171_AT, 242871_AT, 200878_AT, 242903_AT, 218589_AT, 203760_S_AT, |
| | 203665_AT, 205891_AT, 207610_S_AT, 220307_AT, 239519_AT, 218556_AT, |
| | 218686_S_AT, 229937_X_AT, 203104_AT, 212977_AT, 223939_AT, 224341_X_AT, |
| | 203887_S_AT, 206637_AT, 228766_AT, 1555756_A_AT, 209392_AT, 214581_X_AT, |
| | 237252_AT, 214297_AT, 202727_S_AT, 1405_I_AT, 221060_S_AT, 202609_AT, |
| | 214724_AT, 226218_AT, 205898_AT, 205099_S_AT, 211661_X_AT, 210895_S_AT, |
| | 222062_AT, 204116_AT, 232746_AT, 225207_AT, 210146_X_AT, 235944_AT, |
| | 205419_AT, 232068_S_AT, 209906_AT, 243483_AT, 210510_S_AT, 204655_AT, |
| | 214054_AT, 211676_S_AT, 208121_S_AT, 1552798_A_AT, 210264_AT, |
| | 206675_S_AT, 205718_AT, 203888_AT, 237904_AT, 203761_AT, 210258_AT, |
| | 210004_AT, 1555759_A_AT, 241929_AT, 201125_S_AT, 219434_AT, 202638_S_AT |
| GO: 0004872~receptor | 220333_AT, 229221_AT, 204912_AT, 212298_AT, 209555_S_AT, 223660_AT, |
| activity | 206206_AT, 222838_AT, 222877_AT, 205798_AT, 206488_S_AT, 206171_AT, |
| | 242871_AT, 242903_AT, 218589_AT, 205891_AT, 207610_S_AT, 220307_AT, |
| | 239519_AT, 218856_AT, 218686_S_AT, 229937_X_AT, 203104_AT, 212977_AT, |
| | 223939_AT, 224341_X_AT, 203887_S_AT, 228766_AT, 206637_AT, 1555756_A_AT, |
| | 209392_AT, 214581_X_AT, 237252_AT, 202727_S_AT, 221060_S_AT, 202609_AT, |
| | 226218_AT, 205898_AT, 205099_S_AT, 211661_X_AT, 210895_S_AT, 222062_AT, |
| | 204116_AT, 232746_AT, 210146_X_AT, 235944_AT, 205419_AT, 232068_S_AT, |
| | 209906_AT, 243483_AT, 210510_S_AT, 210264_AT, 208121_S_AT, 211676_S_AT, |
| | 1552798_A_AT, 205718_AT, 237904_AT, 203888_AT, 210004_AT, 241929_AT, |
| | 201125_S_AT, 219434_AT, 202638_S_AT |
| GO: 0044425~membrane | 1553995_A_AT, 220333_AT, 210495_X_AT, 229221_AT, 223723_AT, 212298_AT, |
| part | 209555_S_AT, 202435_S_AT, 223660_AT, 222838_AT, 211066_X_AT, 219412_AT, |
| | 205798_AT, 224480_S_AT, 214255_AT, 242871_AT, 238681_AT, 228579_AT, |
| | 230360_AT, 226545_AT, 227747_AT, 242903_AT, 228490_AT, 207610_S_AT, |
| | 205505_AT, 220307_AT, 226629_AT, 216080_S_AT, 203922_S_AT, 218686_S_AT, |
| | 229937_X_AT, 221815_AT, 214830_AT, 227052_AT, 203104_AT, 212977_AT, |
| | 202756_S_AT, 228766_AT, 230925_AT, 201005_AT, 201242_S_AT, 242907_AT |
| | 209122_AT, 205128_X_AT, 214581_X_AT, 237252_AT, 237030_AT, 214297_AT, |
| | 63825_AT, 202727_S_AT, 221060_S_AT, 211719_X_AT, 223596_AT, 223501_AT, |
| | 205898_AT, 34210_AT, 205099_S_AT, 205566_AT, 219574_AT, 218613_AT, |
| | 210146_X_AT, 215836_S_AT, 217678_AT, 232068_S_AT, 240076_AT, 209047_AT, |
| | 238581_AT, 57715_AT, 209906_AT, 243483_AT, 210510_S_AT, 211030_S_AT, |
| | 235458_AT, 219926_AT, 208121_S_AT, 211676_S_AT, 238669_AT, 204257_AT, |
| | 223434_AT, 224989_AT, 204105_S_AT, 204881_S_AT, 206134_AT, 205306_X_AT, |
| | 219994_AT, 201125_S_AT, 203355_S_AT, 202638_S_AT, 239761_AT, 201243_S_AT, |
| | 226136_AT, 204912_AT, 238638_AT, 206206_AT, 228918_AT, 222877_AT, |
| | 227134_AT, 203217_S_AT, 212062_AT, 202434_S_AT, 222858_S_AT, 201278_AT, |
| | 229900_AT, 204222_S_AT, 244375_AT, 206488_S_AT, 241392_AT, 206171_AT, |
| | 218854_AT, 1553151_AT, 208789_AT, 228640_AT, 203939_AT, 237442_AT, |
| | 205542_AT, 213338_AT, 243894_AT, 218589_AT, 205891_AT, 203665_AT, |
| | 202436_S_AT, 221565_S_AT, 207542_S_AT, 201279_S_AT, 202748_AT, 239519_AT, |
| | 219386_S_AT, 208161_S_AT, 221266_S_AT, 209921_AT, 218856_AT, 210145_AT, |
| | 227889_AT, 223502_S_AT, 225337_AT, 236345_AT, 1557938_S_AT, 205003_AT, |
| | 220484_AT, 203887_S_AT, 224341_X_AT, 223939_AT, 206637_AT, 211138_S_AT, |
| | 1555756_A_AT, 209392_AT, 228762_AT, 209993_AT, 201324_AT, 215813_S_AT, |
| | 212464_S_AT, 1555606_A_AT, 216442_X_AT, 228708_AT, 202609_AT, 214724_AT, |
| | 226218_AT, 204661_AT, 225809_AT, 211661_X_AT, 214841_AT, 210895_S_AT, |
| | 222062_AT, 232746_AT, 204116_AT, 219385_AT, 223092_AT, 225188_AT, |
| | 202827_S_AT, 205419_AT, 87100_AT, 235286_AT, 224990_AT, 235299_AT, |
| | 1560960_AT, 201952_AT, 1554285_AT, 202437_S_AT, 225189_S_AT, |
| | 1552798_A_AT, 210264_AT, 205718_AT, 237904_AT, 203888_AT, 223798_AT, |
| | 210757_X_AT, 210004_AT, 229797_AT, 201280_S_AT, 241929_AT, 219434_AT, |
| | 201951_AT |
| GO: 0016021~integral to | 220333_AT, 229221_AT, 223723_AT, 212298_AT, 209555_S_AT, 223660_AT, |
| membrane | 222838_AT, 211066_X_AT, 205798_AT, 224480_S_AT, 214255_AT, 242871_AT, |
| | 238681_AT, 228579_AT, 230360_AT, 242903_AT, 227747_AT, 228490_AT, |
| | 207610_S_AT, 205505_AT, 220307_AT, 226629_AT, 216080_S_AT, 203922_S_AT, |
| | 218686_S_AT, 229937_X_AT, 221815_AT, 214830_AT, 227052_AT, 203104_AT, |
| | 212977_AT, 202756_S_AT, 228766_AT, 201005_AT, 201242_S_AT, 214581_X_AT, |
| | 237252_AT, 237030_AT, 214297_AT, 63825_AT, 202727_S_AT, 221060_S_AT, |
| | 223536_AT, 223501_AT, 205898_AT, 34210_AT, 205099_S_AT, 205566_AT, |
| | 219574_AT, 210146_X_AT, 215836_S_AT, 217678_AT, 232068_S_AT, 240076_AT, |
| | 209047_AT, 57715_AT, 209906_AT, 243483_AT, 210510_S_AT, 211030_S_AT, |
| | 235458_AT, 219926_AT, 211676_S_AT, 208121_S_AT, 204257_AT, 223434_AT, |
| | 224989_AT, 204105_S_AT, 204881_S_AT, 206134_AT, 205306_X_AT, 201125_S_AT, |
| | 202638_S_AT, 239761_AT, 201243_S_AT, 226136_AT, 204912_AT, 238638_AT, |
| | 206206_AT, 228918_AT, 222877_AT, 203217_S_AT, 212062_AT, 204222_S_AT, |
| | 206488_S_AT, 241392_AT, 206171_AT, 218854_AT, 228640_AT, 205542_AT, |
| | 213338_AT, 218589_AT, 243894_AT, 205891_AT, 207542_S_AT, 221565_S_AT, |
| | 239519_AT, 219386_S_AT, 208161_S_AT, 221266_S_AT, 209921_AT, 218856_AT, |
| | 210145_AT, 227889_AT, 223502_S_AT, 225337_AT, 236345_AT, 220484_AT, |
| | 203887_S_AT, 224341_X_AT, 223939_AT, 206637_AT, 211138_S_AT, |
| | 1555756_A_AT, 209392_AT, 228762_AT, 209993_AT, 201324_AT, 1555606_A_AT, |
| | 202609_AT, 226218_AT, 204661_AT, 225809_AT, 211661_X_AT, 214841_AT, |
| | 210895_S_AT, 222062_AT, 232746_AT, 204116_AT, 219385_AT, 223092_AT, |
| | 202827_S_AT, 205419_AT, 87100_AT, 235286_AT, 224990_AT, 235299_AT, |
| | 1554285_AT, 201952_AT, 1552798_A_AT, 210264_AT, 205718_AT, 237904_AT, |
| | 203888_AT, 223798_AT, 210004_AT, 229797_AT, 241929_AT, 219434_AT, |
| | 201951_AT |
| GO: 0044421~extracellular | 237252_AT, 229221_AT, 216442_X_AT, 210495_X_AT, 201069_AT, 212171_X_AT, |
| region part | 213503_X_AT, 204575_S_AT, 1405_I_AT, 210427_X_AT, 211719_X_AT, 223501_AT, |
| | 229900_AT, 207433_AT, 227265_AT, 203936_S_AT, 235944_AT, 210512_S_AT, |
| | 202827_S_AT, 221463_AT, 226545_AT, 211527_X_AT, 204475_AT, 235286_AT, |
| | 37145_AT, 203665_AT, 213428_S_AT, 217757_AT, 229004_AT, 208816_X_AT, |
| | 204655_AT, 210513_S_AT, 201590_X_AT, 212190_AT, 223502_S_AT, 203888_AT, |
| | 212143_S_AT, 203887_S_AT, 202756_S_AT, 204834_AT, 205495_S_AT, 228873_AT, |
| | 1555759_A_AT, 1556423_AT, 210095_S_AT, 202638_S_AT, 212464_S_AT |
| GO: 0004888~transmembrane | 237252_AT, 204912_AT, 212298_AT, 209555_S_AT, 223660_AT, 202727_S_AT, |
| receptor activity | 221060_S_AT, 222877_AT, 226218_AT, 205898_AT, 205099_S_AT, 211661_X_AT, |
| | 205798_AT, 206488_S_AT, 222062_AT, 204116_AT, 232746_AT, 206171_AT, |
| | 235944_AT, 232068_S_AT, 205419_AT, 242903_AT, 218589_AT, 209906_AT, |
| | 205891_AT, 207610_S_AT, 210510_S_AT, 239519_AT, 210264_AT, 1552798_A_AT, |
| | 208121_S_AT, 211676_S_AT, 237904_AT, 203888_AT, 203104_AT, 212977_AT, |
| | 203887_S_AT, 224341_X_AT, 223939_AT, 210004_AT, 228766_AT, 206637_AT, |
| | 209392_AT, 241929_AT, 202638_S_AT |
| GO: 0006955~immune | 214211_AT, 202869_AT, 212171_X_AT, 205552_S_AT, 1405_I_AT, 221060_S_AT, |
| response | 206206_AT, 222838_AT, 226218_AT, 223501_AT, 205099_S_AT, 211661_X_AT, |
| | 205798_AT, 210895_S_AT, 222062_AT, 204116_AT, 207433_AT, 210146_X_AT, |
| | 210512_S_AT, 232068_S_AT, 221463_AT, 220066_AT, 205419_AT, 211527_X_AT, |
| | 238581_AT, 202748_AT, 204655_AT, 210513_S_AT, 1552798_A_AT, 203922_S_AT, |
| | 229937_X_AT, 223502_S_AT, 206157_AT, 223434_AT, 224341_X_AT, 210004_AT, |
| | 206637_AT, 1555756_A_AT, 1555759_A_AT, 209392_AT, 219434_AT, 242907_AT, |
| | 202638_S_AT, 213293_S_AT |
| GO: 0009611~response | 210495_X_AT, 229221_AT, 212298_AT, 209555_S_AT, 223660_AT, 206206_AT, |
| to wounding | 206488_S_AT, 206171_AT, 228490_AT, 203665_AT, 239519_AT, 203922_S_AT, |
| | 221815_AT, 206157_AT, 225337_AT, 224341_X_AT, 203887_S_AT, 228766_AT, |
| | 1555756_A_AT, 201005_AT, 203060_S_AT, 212464_S_AT, 237252_AT, |
| | 216442_X_AT, 63825_AT, 1405_I_AT, 221060_S_AT, 211719_X_AT, 205898_AT, |
| | 205099_S_AT, 211661_X_AT, 205566_AT, 207433_AT, 211026_S_AT, 235944_AT, |
| | 221463_AT, 87100_AT, 232068_S_AT, 208436_S_AT, 204465_S_AT, 209906_AT, |
| | 217757_AT, 210510_S_AT, 204655_AT, 1552553_A_AT, 1552798_A_AT, 203888_AT, |
| | 237904_AT, 210004_AT, 1555759_A_AT, 241929_AT |
| GO: 0009605~response | 229221_AT, 210495_X_AT, 212298_AT, 209555_S_AT, 223660_AT, 206206_AT, |
| to external stimulus | 222877_AT, 206488_S_AT, 206171_AT, 202284_S_AT, 220066_AT, 228490_AT, |
| | 203665_AT, 205831_AT, 239519_AT, 203922_S_AT, 210145_AT, 206157_AT, |
| | 221815_AT, 225337_AT, 224341_X_AT, 203887_S_AT, 228766_AT, 1555755_A_AT, |
| | 209392_AT, 203060_S_AT, 201005_AT, 212464_S_AT, 237252_AT, 216442_X_AT, |
| | 63825_AT, 1405_I_AT, 221060_S_AT, 211719_X_AT, 205898_AT, 205099_S_AT, |
| | 211661_X_AT, 205566_AT, 207433_AT, 211026_S_AT, 235944_AT, 202827_S_AT, |
| | 208436_S_AT, 221463_AT, 87100_AT, 232068_S_AT, 235286_AT, 204465_S_AT, |
| | 209906_AT, 217757_AT, 210510_S_AT, 204655_AT, 1552553_A_AT, 1552798_A_AT, |
| | 237904_AT, 203888_AT, 208712_AT, 210004_AT, 1555759_A_AT, 241929_AT |
| GO: 0002376~immune | 202869_AT, 205552_S_AT, 206206_AT, 222838_AT, 205798_AT, 203936_S_AT, |
| system process | 220066_AT, 200878_AT, 202748_AT, 201565_S_AT, 221266_S_AT, 203922_S_AT, |
| | 229937_X_AT, 223502_S_AT, 215990_S_AT, 206157_AT, 201422_AT, 224341_X_AT, |
| | 206637_AT, 1555756_A_AT, 209392_AT, 242907_AT, 213293_S_AT, 214211_AT, |
| | 212171_X_AT, 1405_I_AT, 201566_X_AT, 221060_S_AT, 226218_AT, 223501_AT, |
| | 205099_S_AT, 211661_X_AT, 210895_S_AT, 222062_AT, 204116_AT, 207433_AT, |
| | 210146_X_AT, 210512_S_AT, 221463_AT, 205419_AT, 232068_S_AT, 211527_X_AT, |
| | 238581_AT, 235286_AT, 209906_AT, 203140_AT, 204655_AT, 210513_S_AT, |
| | 1552798_A_AT, 208978_AT, 213931_AT, 223434_AT, 210004_AT, 1555759_A_AT, |
| | 219434_AT, 202638_S_AT |
| GO: 0050896~response | 210495_X_AT, 229221_AT, 202869_AT, 212298_AT, 209555_S_AT, 202435_S_AT, |
| to stimulus | 223560_AT, 222838_AT, 205798_AT, 1563621_AT, 202284_S_AT, 242903_AT, |
| | 228490_AT, 201565_S_AT, 208922_S_AT, 229937_X_AT, 215990_S_AT, 221815_AT, |
| | 205495_S_AT, 228766_AT, 201005_AT, 203060_S_AT, 201242_S_AT, 242907_AT, |
| | 209122_AT, 213293_S_AT, 205128_X_AT, 214211_AT, 237252_AT, 212171_X_AT, |
| | 63825_AT, 202727_S_AT, 1405_I_AT, 221060_S_AT, 201566_X_AT, 211719_X_AT, |
| | 223501_AT, 205898_AT, 204058_AT, 205099_S_AT, 205566_AT, 207433_AT, |
| | 210146_X_AT, 235944_AT, 217678_AT, 209047_AT, 221463_AT, 232068_S_AT, |
| | 208436_S_AT, 238581_AT, 209906_AT, 1554992_AT, 243483_AT, 217757_AT, |
| | 204059_S_AT, 210510_S_AT, 210513_AT, 211676_S_AT, 238669_AT, 223434_AT, |
| | 225631_AT, 225097_AT, 205306_X_AT, 202638_S_AT, 201243_S_AT, 201069_AT, |
| | 205552_S_AT, 227948_AT, 206206_AT, 222877_AT, 202434_S_AT, 206488_S_AT, |
| | 206171_AT, 220066_AT, 200878_AT, 37145_AT, 205891_AT, 203665_AT, |
| | 202436_S_AT, 207542_S_AT, 214453_S_AT, 202748_AT, 239519_AT, 209921_AT, |
| | 210145_AT, 206157_AT, 223502_S_AT, 225337_AT, 220484_AT, 224341_X_AT, |
| | 203887_S_AT, 206637_AT, 211138_S_AT, 1555756_A_AT, 209332_AT, 209993_AT, |
| | 215813_S_AT, 212464_S_AT, 216442_X_AT, 202609_AT, 226218_AT, 218934_S_AT, |
| | 211661_X_AT, 210895_S_AT, 222062_AT, 204116_AT, 211026_S_AT, 210512_S_AT, |
| | 223092_AT, 202827_S_AT, 87100_AT, 205419_AT, 211527_X_AT, 235286_AT, |
| | 204465_S_AT, 203140_AT, 203980_AT, 204655_AT, 1552553_A_AT, 202437_S_AT, |
| | 230559_X_AT, 1552798_A_AT, 213931_AT, 203888_AT, 237904_AT, 208712_AT, |
| | 210004_AT, 229797_AT, 1555759_A_AT, 241929_AT, 219434_AT |
| GO: 00322501~multicellular | 219358_S_AT, 220333_AT, 229221_AT, 212293_AT, 203555_S_AT, 202435_S_AT, |
| organismal process | 221011_S_AT, 205798_AT, 228579_AT, 203936_S_AT, 242871_AT, 209348_S_AT, |
| | 230360_AT, 202284_S_AT, 227747_AT, 1569149_AT, 201565_S_AT, 201590_X_AT, |
| | 204653_AT, 204999_S_AT, 226066_AT, 215990_S_AT, 203104_AT, 200884_AT, |
| | 228766_AT, 243556_AT, 201005_AT, 203060_S_AT, 210095_S_AT, 205128_X_AT, |
| | 237252_AT, 214297_AT, 212171_X_AT, 221060_S_AT, 201566_X_AT, 228964_AT, |
| | 223596_AT, 218502_S_AT, 206835_AT, 207433_AT, 235944_AT, 222670_S_AT, |
| | 209047_AT, 232068_S_AT, 209906_AT, 1554992_AT, 243483_AT, 210510_S_AT, |
| | 210513_S_AT, 206675_S_AT, 238669_AT, 204105_S_AT, 212614_AT, 204881_S_AT, |
| | 225097_AT, 201069_AT, 224218_S_AT, 222877_AT, 202434_S_AT, 201278_AT, |
| | 206488_S_AT, 244375_AT, 211962_S_AT, 220066_AT, 200878_AT, 204141_AT, |
| | 205891_AT, 203665_AT, 202436_S_AT, 207542_S_AT, 201279_S_AT, 204998_S_AT, |
| | 208816_X_AT, 239519_AT, 221266_S_AT, 233540_S_AT, 210145_AT, 212190_AT, |
| | 212143_S_AT, 212086_X_AT, 220484_AT, 224341_X_AT, 203887_S_AT, |
| | 1555756_A_AT, 228762_AT, 222651_S_AT, 211986_AT, 213265_AT, 201324_AT, |
| | 215813_S_AT, 213503_X_AT, 204575_S_AT, 227240_AT, 210427_X_AT, 202609_AT, |
| | 214724_AT, 226218_AT, 244579_AT, 218559_S_AT, 211661_X_AT, 207233_S_AT, |
| | 204684_AT, 210512_S_AT, 223092_AT, 202827_S_AT, 205419_AT, 204475_AT, |
| | 211527_X_AT, 204465_S_AT, 203140_AT, 1560960_AT, 1569150_X_AT, 201952_AT, |
| | 203980_AT, 220935_S_AT, 1552553_A_AT, 202437_S_AT, 1552798_A_AT, |
| | 208978_AT, 213931_AT, 203888_AT, 208712_AT, 210757_X_AT, 222876_S_AT, |
| | 210004_AT, 229797_AT, 201280_S_AT, 241929_AT, 201951_AT |
| GO: 0050793~regulation | 210495_X_AT, 212298_AT, 209555_S_AT, 227948_AT, 205798_AT, 206488_S_AT, |
| of developmental | 214255_AT, 209348_S_AT, 1569149_AT, 203665_AT, 205891_AT, 204998_S_AT, |
| process | 201565_S_AT, 239519_AT, 233540_S_AT, 204999_S_AT, 226066_AT, 210145_AT, |
| | 215990_S_AT, 212143_S_AT, 215602_AT, 224341_X_AT, 228766_AT, 210095_S_AT, |
| | 212464_S_AT, 216442_X_AT, 212171_X_AT, 1553906_S_AT, 1405_I_AT, |
| | 201566_X_AT, 221060_S_AT, 211719_X_AT, 226218_AT, 218559_S_AT, |
| | 207233_S_AT, 210895_S_AT, 206835_AT, 222062_AT, 204116_AT, 207433_AT, |
| | 210512_S_AT, 223092_AT, 222670_S_AT, 232068_S_AT, 211527_X_AT, 203140_AT, |
| | 1569150_X_AT, 220935_S_AT, 210510_S_AT, 204655_AT, 210513_S_AT, |
| | 230559_X_AT, 1565754_X_AT, 1552798_A_AT, 213931_AT, 1565752_AT, |
| | 208712_AT, 204105_S_AT, 1555759_A_AT, 1556423_AT, 241929_AT |
| GO: 0009897~external | 229221_AT, 243483_AT, 214297_AT, 201952_AT, 220307_AT, 221060_S_AT, |
| side of plasma | 1552798_A_AT, 226218_AT, 205798_AT, 204105_S_AT, 210895_S_AT, 204116_AT, |
| membrane | 224341_X_AT, 202756_S_AT, 1555756_A_AT, 232068_S_AT, 201951_AT, |
| | 202638_S_AT |
| GO: 0051239~regulation | 212298_AT, 223660_AT, 205798_AT, 206171_AT, 209348_S_AT, 220066_AT, |
| of multicellular | 200878_AT, 1569149_AT, 203665_AT, 205891_AT, 204998_S_AT, 208816_X_AT, |
| organismal process | 201565_S_AT, 239519_AT, 201590_X_AT, 233540_S_AT, 204999_S_AT, 226066_AT, |
| | 210145_AT, 215990_S_AT, 212143_S_AT, 224341_X_AT, 1555756_A_AT, |
| | 215813_S_AT, 210095_S_AT, 205128_X_AT, 212171_X_AT, 213503_X_AT, |
| | 1405_I_AT, 201566_X_AT, 210427_X_AT, 221060_S_AT, 226218_AT, 218934_S_AT, |
| | 218559_S_AT, 207233_S_AT, 210895_S_AT, 206835_AT, 222062_AT, 204116_AT, |
| | 207433_AT, 210512_S_AT, 223092_AT, 222670_S_AT, 232068_S_AT, 211527_X_AT, |
| | 1554992_AT, 203140_AT, 1569150_X_AT, 220935_S_AT, 210510_S_AT, 204655_AT, |
| | 210513_S_AT, 1552798_A_AT, 213931_AT, 238669_AT, 237904_AT, 204105_S_AT, |
| | 208712_AT, 1555759_A_AT, 1556423_AT |
| GO: 0006952~defense | 229221_AT, 216442_X_AT, 210495_X_AT, 223660_AT, 1405_I_AT, 221060_S_AT, |
| response | 206206_AT, 211719_X_AT, 222838_AT, 205898_AT, 205099_S_AT, 211661_X_AT, |
| | 222062_AT, 206171_AT, 207433_AT, 211026_S_AT, 210146_X_AT, 208436_S_AT, |
| | 232068_S_AT, 221463_AT, 220066_AT, 37145_AT, 209906_AT, 203665_AT, |
| | 205891_AT, 217757_AT, 204655_AT, 1552553_A_AT, 1552798_A_AT, 203922_S_AT, |
| | 206157_AT, 237904_AT, 210004_AT, 224341_X_AT, 205495_S_AT, 1555756_A_AT, |
| | 1555759_A_AT, 212464_S_AT |
| GO: 0006954~inflammatory | 210495_X_AT, 216442_X_AT, 229221_AT, 223660_AT, 1405_I_AT, 206206_AT, |
| response | 221060_S_AT, 211719_X_AT, 205099_S_AT, 211661_X_AT, 211026_S_AT, |
| | 206171_AT, 207433_AT, 232068_S_AT, 221463_AT, 208436_S_AT, 209906_AT, |
| | 203665_AT, 217757_AT, 204655_AT, 1552553_A_AT, 1552798_A_AT, 203922_S_AT, |
| | 206157_AT, 237904_AT, 224341_X_AT, 210004_AT, 1555756_A_AT, 1555759_A_AT, |
| | 212464_S_AT |
| GO: 0031012~extracellular | 229221_AT, 216442_X_AT, 210495_X_AT, 201069_AT, 213428_S_AT, 212171_X_AT, |
| matrix | 213503_X_AT, 204575_S_AT, 208816_X_AT, 229004_AT, 210513_S_AT, |
| | 210427_X_AT, 201590_X_AT, 211719_X_AT, 202756_S_AT, 228873_AT, |
| | 203936_S_AT, 235944_AT, 210512_S_AT, 202827_S_AT, 211527_X_AT, 204475_AT, |
| | 212464_S_AT |
| GO: 0048583~regulation | 1553995_A_AT, 212171_X_AT, 213503_X_AT, 227948_AT, 1405_I_AT, |
| of response to stimulus | 210427_X_AT, 221060_S_AT, 226218_AT, 223501_AT, 205798_AT, 210895_S_AT, |
| | 222062_AT, 207433_AT, 210512_S_AT, 220066_AT, 232068_S_AT, 208436_S_AT, |
| | 211527_X_AT, 203939_AT, 209906_AT, 203665_AT, 205891_AT, 203140_AT, |
| | 217757_AT, 203980_AT, 208816_X_AT, 204655_AT, 210513_S_AT, 230559_X_AT, |
| | 201590_X_AT, 1552798_A_AT, 210145_AT, 223502_S_AT, 215990_S_AT, |
| | 224341_X_AT, 225097_AT, 1555756_A_AT, 1555759_A_AT, 202638_S_AT |
| GO: 0005578~proteinaceous | 216442_X_AT, 210495_X_AT, 201069_AT, 213428_S_AT, 212171_X_AT, |
| extracellular matrix | 213503_X_AT, 204575_S_AT, 208816_X_AT, 229004_AT, 210513_S_AT, |
| | 210427_X_AT, 201590_X_AT, 211719_X_AT, 202756_S_AT, 228873_AT, |
| | 203936_S_AT, 235944_AT, 210512_S_AT, 202827_S_AT, 211527_X_AT, 204475_AT, |
| | 212464_S_AT |
| GO: 0009986~cell surface | 229221_AT, 214297_AT, 212171_X_AT, 209555_S_AT, 221060_S_AT, 226218_AT, |
| | 205798_AT, 206488_S_AT, 210895_S_AT, 204116_AT, 210512_S_AT, 220066_AT, |
| | 232068_S_AT, 211527_X_AT, 243483_AT, 201952_AT, 220307_AT, 210513_S_AT, |
| | 221266_S_AT, 1552798_A_AT, 204105_S_AT, 224341_X_AT, 202756_S_AT, |
| | 228766_AT, 1555756_A_AT, 241929_AT, 209993_AT, 201951_AT, 202638_S_AT |
| GO: 0002682~regulation | 229221_AT, 212171_X_AT, 1405_I_AT, 201566_X_AT, 221060_S_AT, 226218_AT, |
| of immune system | 223501_AT, 218559_S_AT, 205798_AT, 207233_S_AT, 210895_S_AT, 222062_AT, |
| process | 204116_AT, 207433_AT, 210512_S_AT, 202284_S_AT, 222670_S_AT, 220066_AT, |
| | 232068_S_AT, 211527_X_AT, 209906_AT, 203665_AT, 205891_AT, 203140_AT, |
| | 217757_AT, 204655_AT, 210513_S_AT, 201565_S_AT, 1552798_A_AT, 226066_AT, |
| | 223502_S_AT, 213931_AT, 215990_S_AT, 224341_X_AT, 1555756_A_AT, |
| | 1555759_A_AT, 202638_S_AT |
| GO: 0005615~extracellular | 237252_AT, 210495_X_AT, 216442_X_AT, 201069_AT, 212171_X_AT, 1405_I_AT, |
| space | 211719_X_AT, 223501_AT, 229900_AT, 207433_AT, 227265_AT, 203936_S_AT, |
| | 210512_S_AT, 226545_AT, 221463_AT, 211527_X_AT, 235286_AT, 37145_AT, |
| | 203665_AT, 217757_AT, 204655_AT, 210513_S_AT, 212190_AT, 223502_S_AT, |
| | 212143_S_AT, 203888_AT, 203887_S_AT, 202756_S_AT, 205495_S_AT, 204834_AT, |
| | 1555759_A_AT, 1556423_AT, 210095_S_AT, 202638_S_AT, 212464_S_AT |
| GO: 0048731~system | 219358_S_AT, 229221_AT, 201069_AT, 212298_AT, 224218_S_AT, 222877_AT, |
| development | 205798_AT, 244375_AT, 211962_S_AT, 209348_S_AT, 203936_S_AT, 202284_S_AT, |
| | 230360_AT, 200878_AT, 204141_AT, 1569149_AT, 203665_AT, 204998_S_AT, |
| | 206816_X_AT, 201565_S_AT, 239519_AT, 201590_X_AT, 221266_S_AT, |
| | 233540_S_AT, 204999_S_AT, 204653_AT, 210145_AT, 226066_AT, 212190_AT, |
| | 215990_S_AT, 212143_S_AT, 212086_X_AT, 220484_AT, 200884_AT, 228762_AT, |
| | 243556_AT, 203060_S_AT, 201005_AT, 222651_S_AT, 211986_AT, 210095_S_AT, |
| | 201324_AT, 213503_X_AT, 214297_AT, 212171_X_AT, 204575_S_AT, 227240_AT, |
| | 201566_X_AT, 210427_X_AT, 228964_AT, 226218_AT, 244579_AT, 218559_S_AT, |
| | 207233_S_AT, 206835_AT, 218502_S_AT, 204684_AT, 207433_AT, 202827_S_AT, |
| | 223092_AT, 222670_S_AT, 210512_S_AT, 205419_AT, 211527_X_AT, 204465_S_AT, |
| | 1554992_AT, 203140_AT, 1560960_AT, 1569150_X_AT, 201952_AT, 220935_S_AT, |
| | 210510_S_AT, 210513_S_AT, 208978_AT, 213931_AT, 204105_S_AT, 208712_AT, |
| | 212614_AT, 204881_S_AT, 222876_S_AT, 229797_AT, 201951_AT |
| GO: 0051707~response | 237252_AT, 1405_I_AT, 202727_S_AT, 221060_S_AT, 211661_X_AT, 222062_AT, |
| to other organism | 207433_AT, 232068_S_AT, 220066_AT, 208436_S_AT, 242903_AT, 37145_AT, |
| | 214453_S_AT, 204655_AT, 1552553_A_AT, 1552798_A_AT, 211676_S_AT, |
| | 229937_X_AT, 210145_AT, 206157_AT, 203888_AT, 224341_X_AT, 203887_S_AT, |
| | 205495_S_AT, 1555756_A_AT, 1555759_A_AT, 213293_S_AT |
| GO: 0007275~multicellular | 220333_AT, 219358_S_AT, 229221_AT, 201069_AT, 212298_AT, 224218_S_AT, |
| organismal | 221011_S_AT, 222877_AT, 201278_AT, 205798_AT, 244375_AT, 211962_S_AT, |
| development | 209348_S_AT, 242871_AT, 203936_S_AT, 202284_S_AT, 230360_AT, 200878_AT, |
| | 204141_AT, 1569149_AT, 203665_AT, 201279_S_AT, 204998_S_AT, 206816_X_AT, |
| | 201565_S_AT, 239519_AT, 201590_X_AT, 221266_S_AT, 233540_S_AT, |
| | 204999_S_AT, 204653_AT, 210145_AT, 226066_AT, 212190_AT, 215990_S_AT, |
| | 212143_S_AT, 203104_AT, 212086_X_AT, 220484_AT, 200884_AT, 203887_S_AT, |
| | 228762_AT, 243556_AT, 203060_S_AT, 201005_AT, 222651_S_AT, 211986_AT, |
| | 210095_S_AT, 201324_AT, 237252_AT, 212171_X_AT, 213503_X_AT, 214297_AT, |
| | 204575_S_AT, 227240_AT, 201566_X_AT, 210427_X_AT, 228964_AT, 214724_AT, |
| | 226218_AT, 244579_AT, 218559_S_AT, 207233_S_AT, 206835_AT, 218502_S_AT, |
| | 204684_AT, 207433_AT, 202827_S_AT, 223092_AT, 210512_S_AT, 222670_S_AT, |
| | 205419_AT, 211527_X_AT, 204465_S_AT, 1554992_AT, 203140_AT, 1560960_AT, |
| | 1569150_X_AT, 201952_AT, 220935_S_AT, 210510_S_AT, 210513_S_AT, |
| | 208978_AT, 206675_S_AT, 213931_AT, 203888_AT, 204105_S_AT, 208712_AT, |
| | 212614_AT, 204881_S_AT, 210757_X_AT, 222876_S_AT, 225097_AT, 229797_AT, |
| | 201280_S_AT, 201951_AT |
| GO: 0032101~regulation | 1553995_A_AT, 205891_AT, 203140_AT, 212171_X_AT, 213503_X_AT, 217757_AT, |
| of response to external | 203980_AT, 208816_X_AT, 1405_I_AT, 204655_AT, 210513_S_AT, 221060_S_AT, |
| stimulus | 210427_X_AT, 201590_X_AT, 1552798_A_AT, 210145_AT, 215990_S_AT, |
| | 224341_X_AT, 207433_AT, 210512_S_AT, 1555759_A_AT, 232068_S_AT, |
| | 211527_X_AT, 203939_AT |
| GO: 0022610~biological | 229221_AT, 210495_X_AT, 216442_X_AT, 212298_AT, 209555_S_AT, 1405_I_AT, |
| adhesion | 211719_X_AT, 222838_AT, 210360_S_AT, 222877_AT, 211066_X_AT, 205898_AT, |
| | 205099_S_AT, 206488_S_AT, 215836_S_AT, 225188_AT, 228640_AT, 227747_AT, |
| | 213428_S_AT, 201952_AT, 204655_AT, 210510_S_AT, 225189_S_AT, 239519_AT, |
| | 203037_S_AT, 205718_AT, 216250_S_AT, 204105_S_AT, 210004_AT, 228766_AT, |
| | 1555756_A_AT, 228873_AT, 1555759_A_AT, 241929_AT, 201125_S_AT, 201005_AT, |
| | 201951_AT, 202638_S_AT, 212464_S_AT |
| GO: 0007155~cell | 229221_AT, 210495_X_AT, 216442_X_AT, 212298_AT, 209555_S_AT, 1405_I_AT, |
| adhesion | 211719_X_AT, 222838_AT, 210360_S_AT, 222877_AT, 211066_X_AT, 205898_AT, |
| | 205099_S_AT, 206488_S_AT, 215836_S_AT, 225188_AT, 228640_AT, 227747_AT, |
| | 213428_S_AT, 201952_AT, 204655_AT, 210510_S_AT, 225189_S_AT, 239519_AT, |
| | 203037_S_AT, 205718_AT, 216250_S_AT, 204105_S_AT, 210004_AT, 228766_AT, |
| | 1555756_A_AT, 228873_AT, 1555759_A_AT, 241929_AT, 201125_S_AT, 201005_AT, |
| | 201951_AT, 202638_S_AT, 212464_S_AT |
| GO: 0048856~anatomical | 219358_S_AT, 210495_X_AT, 229221_AT, 201069_AT, 212238_AT, 224218_S_AT, |
| structure development | 222877_AT, 201278_AT, 205798_AT, 244375_AT, 211962_S_AT, 209348_S_AT, |
| | 203936_S_AT, 202284_S_AT, 230360_AT, 200878_AT, 204141_AT, 1569149_AT, |
| | 203665_AT, 201279_S_AT, 204998_S_AT, 208816_X_AT, 201565_S_AT, 239519_AT, |
| | 201590_X_AT, 221266_S_AT, 233540_S_AT, 204999_S_AT, 204653_AT, 210145_AT, |
| | 226066_AT, 212190_AT, 215990_S_AT, 212143_S_AT, 212086_X_AT, 220484_AT, |
| | 200884_AT, 228762_AT, 243556_AT, 203060_S_AT, 201005_AT, 222651_S_AT, |
| | 211986_AT, 210095_S_AT, 201324_AT, 212464_S_AT, 216442_X_AT, 213503_X_AT, |
| | 214297_AT, 212171_X_AT, 204575_S_AT, 227240_AT, 201566_X_AT, 210427_X_AT, |
| | 228964_AT, 211719_X_AT, 226218_AT, 244579_AT, 218559_S_AT, 207233_S_AT, |
| | 206835_AT, 218502_S_AT, 204684_AT, 207433_AT, 202827_S_AT, 223092_AT, |
| | 222670_S_AT, 210512_S_AT, 205419_AT, 211527_X_AT, 204465_S_AT, |
| | 1554992_AT, 203140_AT, 1560960_AT, 1569150_X_AT, 201952_AT, 220935_S_AT, |
| | 210510_S_AT, 210513_S_AT, 208978_AT, 213931_AT, 204105_S_AT, 208712_AT, |
| | 212614_AT, 204881_S_AT, 210757_X_AT, 222876_S_AT, 229797_AT, 201280_S_AT, |
| | 201951_AT |
| GO: 0065008~regulation | 223723_AT, 210495_X_AT, 212298_AT, 209555_S_AT, 202435_S_AT, 227948_AT, |
| of biological quality | 205798_AT, 201278_AT, 202434_S_AT, 206488_S_AT, 214255_AT, 202284_S_AT, |
| | 200878_AT, 203665_AT, 205891_AT, 202436_S_AT, 201279_S_AT, 208816_X_AT, |
| | 201565_S_AT, 239519_AT, 201590_X_AT, 210145_AT, 223502_S_AT, 215990_S_AT, |
| | 200884_AT, 215602_AT, 203887_S_AT, 224341_X_AT, 201422_AT, 228766_AT, |
| | 203060_S_AT, 201005_AT, 215813_S_AT, 201324_AT, 205128_X_AT, 212464_S_AT, |
| | 214211_AT, 237252_AT, 216442_X_AT, 212171_X_AT, 213503_X_AT, |
| | 1553906_S_AT, 1405_I_AT, 221060_S_AT, 201566_X_AT, 210427_X_AT, |
| | 211719_X_AT, 223596_AT, 226218_AT, 223501_AT, 34210_AT, 204661_AT, |
| | 205099_S_AT, 206835_AT, 207433_AT, 235944_AT, 210512_S_AT, 208436_S_AT, |
| | 232068_S_AT, 211527_X_AT, 209906_AT, 1554992_AT, 203140_AT, 243483_AT, |
| | 203980_AT, 204655_AT, 210510_S_AT, 210513_S_AT, 202437_S_AT, 230559_X_AT, |
| | 1565754_X_AT, 1552738_A_AT, 213931_AT, 203888_AT, 238669_AT, 1565752_AT, |
| | 210757_X_AT, 1555759_A_AT, 241929_AT, 201280_S_AT, 223394_AT |
| GO: 0007166~cell surface | 212298_AT, 223660_AT, 210360_S_AT, 242794_AT, 205798_AT, 244375_AT, |
| receptor linked signal | 206171_AT, 232333_AT, 218589_AT, 205891_AT, 207610_S_AT, 239519_AT, |
| transduction | 234645_AT, 226066_AT, 203104_AT, 212977_AT, 223939_AT, 206637_AT, |
| | 1555756_A_AT, 209392_AT, 212171_X_AT, 1405_I_AT, 235457_AT, 202609_AT, |
| | 214724_AT, 226218_AT, 205838_AT, 205099_S_AT, 211661_X_AT, 207233_S_AT, |
| | 222062_AT, 232746_AT, 210146_X_AT, 210512_S_AT, 205419_AT, 211527_X_AT, |
| | 244414_AT, 209906_AT, 242405_AT, 210510_S_AT, 204655_AT, 210513_S_AT, |
| | 214054_AT, 203037_S_AT, 210264_AT, 205718_AT, 237904_AT, 208712_AT, |
| | 212614_AT, 210258_AT, 206134_AT, 225097_AT, 1555759_A_AT, 201125_S_AT |
| GO: 0050727~regulation | 1553995_A_AT, 203140_AT, 205891_AT, 217757_AT, 203980_AT, 1405_I_AT, |
| of inflammatory | 204655_AT, 221060_S_AT, 1552798_A_AT, 210145_AT, 215990_S_AT, |
| response | 224341_X_AT, 207433_AT, 1555759_A_AT, 232068_S_AT, 203939_AT |
| GO: 0006950~response | 210495_X_AT, 229221_AT, 201069_AT, 212298_AT, 209555_S_AT, 227948_AT, |
| to stress | 223660_AT, 206206_AT, 222838_AT, 206488_S_AT, 206171_AT, 202284_S_AT, |
| | 1563621_AT, 220066_AT, 200878_AT, 37145_AT, 228490_AT, 203665_AT, |
| | 205891_AT, 239519_AT, 203922_S_AT, 210145_AT, 215990_S_AT, 221815_AT, |
| | 206157_AT, 225337_AT, 224341_X_AT, 203887_S_AT, 205495_S_AT, 228766_AT, |
| | 211138_S_AT, 1555756_A_AT, 201005_AT, 203060_S_AT, 201242_S_AT, |
| | 215813_S_AT, 212464_S_AT, 205128_X_AT, 237252_AT, 216442_X_AT, |
| | 212171_X_AT, 63825_AT, 1405_I_AT, 221060_S_AT, 211719_X_AT, 218934_S_AT, |
| | 205898_AT, 205099_S_AT, 211661_X_AT, 205566_AT, 222062_AT, 211026_S_AT, |
| | 207433_AT, 210146_X_AT, 235944_AT, 202827_S_AT, 210512_S_AT, 221463_AT, |
| | 87100_AT, 232068_S_AT, 208436_S_AT, 211527_X_AT, 204465_S_AT, 209906_AT, |
| | 203140_AT, 243483_AT, 217757_AT, 210510_S_AT, 204655_AT, 1552553_A_AT, |
| | 230559_X_AT, 210513_S_AT, 1552798_A_AT, 238669_AT, 203888_AT, 237904_AT, |
| | 208712_AT, 210004_AT, 225631_AT, 225097_AT, 205306_X_AT, 1555759_A_AT, |
| | 241929_AT, 201243_S_AT |
| GO: 0032502~developmental | 219358_S_AT, 220333_AT, 229221_AT, 210495_X_AT, 212298_AT, 221011_S_AT, |
| process | 205798_AT, 203936_S_AT, 242871_AT, 209348_S_AT, 230360_AT, 202284_S_AT, |
| | 1569149_AT, 201565_S_AT, 201590_X_AT, 204653_AT, 204999_S_AT, 226066_AT, |
| | 215990_S_AT, 203104_AT, 200884_AT, 243556_AT, 201005_AT, 203060_S_AT, |
| | 210095_S_AT, 205128_X_AT, 237252_AT, 214297_AT, 212171_X_AT, 1405_I_AT, |
| | 201566_X_AT, 211719_X_AT, 228964_AT, 218502_S_AT, 206835_AT, 207433_AT, |
| | 222670_S_AT, 1554992_AT, 210510_S_AT, 210513_S_AT, 206675_S_AT, |
| | 238669_AT, 212614_AT, 204105_S_AT, 204881_S_AT, 225097_AT, 201069_AT, |
| | 224218_S_AT, 222877_AT, 201278_AT, 244375_AT, 211962_S_AT, 200878_AT, |
| | 204141_AT, 203665_AT, 201279_S_AT, 204998_S_AT, 208816_X_AT, 239519_AT, |
| | 221266_S_AT, 233540_S_AT, 212190_AT, 210145_AT, 212143_S_AT, 212086_X_AT, |
| | 220484_AT, 203887_S_AT, 228762_AT, 222651_S_AT, 211986_AT, 201324_AT, |
| | 215813_S_AT, 212464_S_AT, 216442_X_AT, 213503_X_AT, 204575_S_AT, |
| | 227240_AT, 210427_X_AT, 214724_AT, 226218_AT, 244579_AT, 218559_S_AT, |
| | 207233_S_AT, 204684_AT, 210512_S_AT, 223092_AT, 202827_S_AT, 205419_AT, |
| | 211527_X_AT, 204465_S_AT, 203140_AT, 1560960_AT, 1569150_X_AT, 201952_AT, |
| | 203980_AT, 220935_S_AT, 204655_AT, 208978_AT, 213931_AT, 203888_AT, |
| | 208712_AT, 222876_S_AT, 210757_X_AT, 229797_AT, 1555759_A_AT, |
| | 201280_S_AT, 201951_AT |
| GO: 0031347~regulation | 1553995_A_AT, 205891_AT, 203140_AT, 217757_AT, 203980_AT, 1405_I_AT, |
| of defense response | 204655_AT, 221060_S_AT, 1552798_A_AT, 210145_AT, 215990_S_AT, |
| | 224341_X_AT, 207433_AT, 1555756_A_AT, 1555759_A_AT, 232068_S_AT, |
| | 208436_S_AT, 220066_AT, 203939_AT |
| GO: 0009607~response | 237252_AT, 1405_I_AT, 202727_S_AT, 221060_S_AT, 218934_S_AT, 211661_X_AT, |
| to biotic stimulus | 222062_AT, 207433_AT, 232068_S_AT, 220066_AT, 208436_S_AT, 242903_AT, |
| | 37145_AT, 214453_S_AT, 204655_AT, 1552553_A_AT, 211676_S_AT, |
| | 1552798_A_AT, 229937_X_AT, 210145_AT, 20615_AT, 203888_AT, 208712_AT, |
| | 224341_X_AT, 203887_S_AT, 205495_S_AT, 1555756_A_AT, 1555759_A_AT, |
| | 213293_S_AT |
| GO: 0019955~cytokine | 204912_AT, 209555_S_AT, 217757_AT, 202727_S_AT, 226218_AT, 211676_S_AT, |
| binding | 205898_S_AT, 205099_S_AT, 205798_AT, 203104_AT, 206488_S_AT, 222062_AT, |
| | 204116_AT, 228766_AT, 241929_AT, 242903_AT |
| GO: 0050776~regulation | 209906_AT, 203665_AT, 205891_AT, 203140_AT, 217757_AT, 221060_S_AT, |
| of immune response | 1552798_A_AT, 226218_AT, 223501_AT, 223502_S_AT, 215590_S_AT, 205798_AT, |
| | 210895_S_AT, 222062_AT, 224341_X_AT, 207433_AT, 1555756_A_AT, 220066_AT, |
| | 232068_S_AT, 202638_S_AT |
| GO: 0040011~locomotion | 210495_X_AT, 216442_X_AT, 229221_AT, 212298_AT, 1405_I_AT, 211719_X_AT, |
| | 222877_AT, 205898_AT, 204268_AT, 205099_S_AT, 211661_X_AT, 207433_AT, |
| | 202827_S_AT, 221463_AT, 235286_AT, 209906_AT, 1560960_AT, 204655_AT, |
| | 210510_S_AT, 239519_AT, 212614_AT, 204105_S_AT, 1555759_A_AT, 209392_AT, |
| | 202638_S_AT, 212464_S_AT |
| GO: 0080134~regulation | 1553995_A_AT, 213503_X_AT, 227948_AT, 1405_I_AT, 210427_X_AT, |
| of response to stress | 221060_S_AT, 207433_AT, 220066_AT, 232068_S_AT, 208436_S_AT, 203939_AT, |
| | 203140_AT, 205891_AT, 217757_AT, 203980_AT, 208816_X_AT, 204655_AT, |
| | 230559_X_AT, 201590_X_AT, 1552798_A_AT, 210145_AT, 215990_S_AT, |
| | 224341_X_AT, 225097_AT, 155575_A_AT, 1555759_A_AT |
| GO: 0007626~locomotory | 209906_AT, 1405_I_AT, 204655_AT, 202609_AT, 222877_AT, 205898_AT, |
| behavior | 205099_S_AT, 211661_X_AT, 220484_AT, 225097_AT, 207433_AT, 229797_AT, |
| | 223092_AT, 1555759_A_AT, 209392_AT, 221463_AT, 235286_AT |
| GO: 0002684~positive | 209906_AT, 205891_AT, 203140_AT, 212171_X_AT, 210513_S_AT, 221060_S_AT, |
| regulation of immune | 226218_AT, 1552798_A_AT, 223501_AT, 223502_S_AT, 215990_S_AT, 205798_AT, |
| system process | 210895_S_AT, 222062_AT, 204116_AT, 224341_X_AT, 1555756_A_AT, |
| | 210512_S_AT, 202284_S_AT, 220066_AT, 232068_S_AT, 211527_X_AT, |
| | 202638_S_AT |
| GO: 0000267~cell | 1553995_A_AT, 202869_AT, 209555_S_AT, 202435_S_AT, 205552_S_AT, |
| fraction | 227134_AT, 211066_X_AT, 222877_AT, 219412_AT, 202434_S_AT, 206488_S_AT, |
| | 208789_AT, 203939_AT, 203665_AT, 202436_S_AT, 216080_S_AT, 201590_X_AT, |
| | 208161_S_AT, 210145_AT, 223502_S_AT, 236345_AT, 1557938_S_AT, |
| | 212086_X_AT, 228766_AT, 209993_AT, 201324_AT, 215813_S_AT, 205128_X_AT, |
| | 213503_X_AT, 1405_I_AT, 210427_X_AT, 202609_AT, 223596_AT, 223501_AT, |
| | 204058_AT, 34210_AT, 204461_AT, 218613_AT, 210146_X_AT, 215836_S_AT, |
| | 1554992_AT, 204059_S_AT, 203980_AT, 204655_AT, 202437_S_AT, 238669_AT, |
| | 204257_AT, 204811_S_AT, 210004_AT, 202087_S_AT, 1555759_A_AT, 241929_AT, |
| | 203355_S_AT |
| GO: 0045028~purinergic | 218589_AT, 205891_AT, 223939_AT, 206637_AT, 223660_AT, 206171_AT, |
| nudeotide receptor | 205419_AT, 237904_AT |
| activity, G-protein | |
| coupled | |
| GO: 0001608~nucleotide | 218589_AT, 205891_AT, 223939_AT, 206637_AT, 223660_AT, 206171_AT, |
| receptor activity, G- | 205419_AT, 237904_AT |
| protein coupled | |
| GO: 0004930~G-protein | 218589_AT, 209906_AT, 205891_AT, 207610_S_AT, 223660_AT, 210264_AT, |
| coupled receptor activity | 205898_AT, 205099_S_AT, 211661_X_AT, 237904_AT, 212977_AT, 232746_AT, |
| | 223939_AT, 206637_AT, 206171_AT, 205419_AT |
| GO: 0016502~nucleotide | 218589_AT, 205891_AT, 223939_AT, 206637_AT, 223660_AT, 206171_AT, |
| receptor activity | 205419_AT, 237904_AT |
| GO: 0001614~purinergic | 218589_AT, 205891_AT, 223939_AT, 206637_AT, 223660_AT, 206171_AT, |
| nucleotide receptor | 205419_AT, 237904_AT |
| GO: 0005626~insoluble | 1553995_A_AT, 202869_AT, 209555_S_AT, 205552_S_AT, 202435_S_AT, |
| friction | 202609_AT, 223596_AT, 222877_AT, 227134_AT, 211066_X_AT, 204661_AT, |
| | 34210_AT, 219412_AT, 202434_S_AT, 218613_AT, 206488_S_AT, 210146_X_AT, |
| | 215836_S_AT, 208789_AT, 203939_AT, 1554992_AT, 203665_AT, 202436_S_AT, |
| | 202437_S_AT, 216080_S_AT, 208161_S_AT, 210145_AT, 1557938_S_AT, |
| | 236345_AT, 238669_AT, 204257_AT, 212086_X_AT, 204881_S_AT, 210004_AT, |
| | 228766_AT, 241929_AT, 203355_S_AT, 209993_AT, 215813_S_AT, 201324_AT, |
| | 205128_X_AT |
| GO: 0048513~organ | 219358_S_AT, 229221_AT, 201069_AT, 212298_AT, 222877_AT, 205798_AT, |
| development | 244375_AT, 211962_S_AT, 203936_S_AT, 209348_S_AT, 202284_S_AT, 200878_AT, |
| | 203665_AT, 208816_K_AT, 239519_AT, 201565_S_AT, 201590_X_AT, 221266_S_AT, |
| | 233540_S_AT, 204653_AT, 226066_AT, 210145_AT, 215990_S_AT, 212086_X_AT, |
| | 220484_AT, 200884_AT, 228762_AT, 201324_AT, 213503_X_AT, 214297_AT, |
| | 212171_X_AT, 204575_S_AT, 210427_X_AT, 201566_X_AT, 228964_AT, 226218_AT, |
| | 218559_S_AT, 207233_S_AT, 206835_AT, 207433_AT, 210512_S_AT, 202827_S_AT, |
| | 222670_S_AT, 205419_AT, 211527_X_AT, 204465_S_AT, 203140_AT, 1560960_AT, |
| | 220935_S_AT, 210510_S_AT, 210513_S_AT, 208978_AT, 213931_AT, 208712_AT, |
| | 212614_AT, 204881_S_AT, 222876_S_AT, 229797_AT |
| GO: 0002697~regulation | 205798_AT, 203665_AT, 205891_AT, 203140_AT, 222062_AT, 217757_AT, |
| of immune effector | 207433_AT, 220066_AT, 226218_AT, 202638_S_AT, 215990_S_AT |
| process | |
| GO: 0051384~response | 208712_AT, 217757_AT, 203980_AT, 207433_AT, 204655_AT, 1405_I_AT, |
| to glucocorticoid | 202284_S_AT, 1555759_A_AT, 215813_S_AT, 210145_AT, 205128_X_AT, |
| stimulus | 238669_AT |
| GO: 0005624~membrane | 1553995_A_AT, 202869_AT, 209555_S_AT, 205552_S_AT, 202435_S_AT, |
| fraction | 202609_AT, 223596_AT, 222877_AT, 227134_AT, 211066_X_AT, 204661_AT, |
| | 34210_AT, 219412_AT, 202434_S_AT, 218613_AT, 206488_S_AT, 210146_X_AT, |
| | 215836_S_AT, 208789_AT, 203939_AT, 1554992_AT, 203665_AT, 202436_S_AT, |
| | 202437_S_AT, 216080_S_AT, 208161_S_AT, 210145_AT, 1557938_S_AT, |
| | 236345_AT, 238669_AT, 204257_AT, 204881_S_AT, 210004_AT, 228766_AT, |
| | 241929_AT, 203355_S_AT, 209993_AT, 215813_S_AT, 201324_AT, 205128_X_AT |
| hsa04060:Cytokine- | 214581_X_AT, 204912_AT, 212171_X_AT, 202727_S_AT, 1405_I_AT, 204655_AT, |
| cytokine receptor | 210513_S_AT, 211676_S_AT, 226218_AT, 223501_AT, 218856_AT, 205898_AT, |
| interaction | 223502_S_AT, 205099_S_AT, 203104_AT, 205798_AT, 204116_AT, 207433_AT, |
| | 210512_S_AT, 1555759_A_AT, 221463_AT, 211527_X_AT, 242903_AT |
| GO: 0031349~positive | 205891_AT, 203980_AT, 1405_I_AT, 204655_AT, 221060_S_AT, 1552798_A_AT, |
| regulation of defense | 210145_AT, 224341_X_AT, 1555756_A_AT, 1555759_A_AT, 232068_S_AT, |
| response | 208436_S_AT, 220066_AT |
| GO: 0048584~positive | 209906_AT, 205891_AT, 212171_X_AT, 203980_AT, 1405_I_AT, 204655_AT, |
| regulation of response to | 210513_S_AT, 221060_S_AT, 1552798_A_AT, 223501_AT, 210145_AT, |
| stimulus | 223502_S_AT, 222062_AT, 224341_X_AT, 225097_AT, 1555756_A_AT, |
| | 210512_S_AT, 1555759_A_AT, 220066_AT, 232068_S_AT, 208436_S_AT, |
| | 211527_X_AT |
| GO: 0031960~response | 208712_AT, 217757_AT, 203980_AT, 207433_AT, 204655_AT, 1405_I_AT, |
| to corticosteroid | 202284_S_AT, 1555759_A_AT, 215813_S_AT, 210145_AT, 205128_X_AT, |
| stimulus | 238669_AT |
| GO: 0042221~response | 229221_AT, 201069_AT, 202435_S_AT, 222877_AT, 202434_S_AT, 202284_S_AT, |
| to chemical stimulus | 220066_AT, 200878_AT, 203665_AT, 207542_S_AT, 202436_S_AT, 201565_S_AT, |
| | 209921_AT, 210145_AT, 224341_X_AT, 203887_S_AT, 1555756_A_AT, 209392_AT, |
| | 201242_S_AT, 209993_AT, 215813_S_AT, 209122_AT, 205128_X_AT, 237252_AT, |
| | 212171_X_AT, 1405_I_AT, 201566_X_AT, 221060_S_AT, 202609_AT, 218934_S_AT, |
| | 204058_AT, 205898_AT, 205099_S_AT, 211661_X_AT, 207433_AT, 217678_AT, |
| | 210512_S_AT, 202827_S_AT, 209047_AT, 221463_AT, 232068_S_AT, 211527_X_AT, |
| | 235286_AT, 209906_AT, 217757_AT, 204059_S_AT, 203980_AT, 204655_AT, |
| | 210513_S_AT, 202437_S_AT, 1552798_A_AT, 213931_AT, 238669_AT, 203888_AT, |
| | 208712_AT, 210004_AT, 1555759_A_AT, 201243_S_AT |
| GO: 0065007~biological | 213891_S_AT, 1553995_A_AT, 223723_AT, 212298_AT, 223660_AT, 222838_AT, |
| regulation | 219412_AT, 205798_AT, 205960_AT, 209348_S_AT, 217997_AT, 232333_AT, |
| | 225171_AT, 220307_AT, 201565_S_AT, 201590_X_AT, 230266_AT, 209684_AT, |
| | 204999_S_AT, 204653_AT, 226066_AT, 215990_S_AT, 221815_AT, 203104_AT, |
| | 200884_AT, 204834_AT, 222146_S_AT, 228766_AT, 210095_S_AT, 205128_X_AT, |
| | 214211_AT, 1560485_AT, 214581_X_AT, 63825_AT, 1405_I_AT, 201566_X_AT, |
| | 228964_AT, 211719_X_AT, 223501_AT, 212387_AT, 205898_AT, 205099_S_AT, |
| | 218502_S_AT, 206835_AT, 207433_AT, 235944_AT, 222670_S_AT, 212382_AT, |
| | 1554992_AT, 217757_AT, 1545754_X_AT, 209606_AT, 211676_S_AT, 206675_S_AT, |
| | 225842_AT, 216250_S_AT, 238669_AT, 1565752_AT, 204105_S_AT, 206134_AT, |
| | 202638_S_AT, 227948_AT, 242794_AT, 202434_S_AT, 222858_S_AT, 206488_S_AT, |
| | 211962_S_AT, 225173_AT, 208789_AT, 220066_AT, 200878_AT, 225166_AT, |
| | 203665_AT, 205891_AT, 208816_X_AT, 239519_AT, 233540_S_AT, 218856_AT, |
| | 210145_AT, 212190_AT, 223502_S_AT, 212143_S_AT, 1557938_S_AT, 215602_AT, |
| | 223939_AT, 203887_S_AT, 206637_AT, 1555756_A_AT, 222651_S_AT, |
| | 215813_S_AT, 218501_AT, 227240_AT, 210427_X_AT, 226218_AT, 218934_S_AT, |
| | 244579_AT, 204661_AT, 207233_S_AT, 210895_S_AT, 222062_AT, 232746_AT, |
| | 227265_AT, 210512_S_AT, 225188_AT, 211527_X_AT, 244414_AT, 203140_AT, |
| | 242405_AT, 1569150_X_AT, 201952_AT, 203980_AT, 225189_S_AT, 230559_X_AT, |
| | 210264_AT, 213931_AT, 210258_AT, 222876_S_AT, 210757_X_AT, 201280_S_AT, |
| | 201951_AT, 219358_S_AT, 210495_X_AT, 229221_AT, 209555_S_AT, 202435_S_AT, |
| | 221011_S_AT, 210360_S_AT, 217999_S_AT, 224480_S_AT, 203936_S_AT, |
| | 228057_AT, 214255_AT, 202284_S_AT, 242903_AT, 1569149_AT, 228490_AT, |
| | 207610_S_AT, 209568_S_AT, 203753_AT, 1553982_A_AT, 212977_AT, 201422_AT, |
| | 230925_AT, 243556_AT, 201005_AT, 203060_S_AT, 213293_S_AT, 237252_AT, |
| | 212171_X_AT, 214297_AT, 202727_S_AT, 235457_AT, 221060_S_AT, 223596_AT, |
| | 34210_AT, 223398_AT, 205566_AT, 218613_AT, 221463_AT, 232068_S_AT, |
| | 208436_S_AT, 209906_AT, 243483_AT, 210510_S_AT, 210513_S_AT, 212614_AT, |
| | 212386_AT, 225097_AT, 219994_AT, 1556423_AT, 203355_S_AT, 224218_S_AT, |
| | 201278_AT, 206171_AT, 1560228_AT, 237442_AT, 203939_AT, 218589_AT, |
| | 202436_S_AT, 201279_S_AT, 204998_S_AT, 234645_AT, 206157_AT, 225337_AT, |
| | 224341_X_AT, 209392_AT, 229435_AT, 201324_AT, 205681_AT, 212464_S_AT, |
| | 216442_X_AT, 213503_X_AT, 1553906_S_AT, 228708_AT, 202609_AT, |
| | 218559_S_AT, 211661_X_AT, 214841_AT, 204116_AT, 225207_AT, 202827_S_AT, |
| | 223092_AT, 205419_AT, 87100_AT, 228368_AT, 220935_S_AT, 204655_AT, |
| | 1552553_A_AT, 202437_S_AT, 203037_S_AT, 214054_AT, 1552798_A_AT, |
| | 208978_AT, 203888_AT, 237904_AT, 208712_AT, 1555759_A_AT, 241929_AT, |
| | 242157_AT, 219434_AT, 223394_AT |
| GO: 0050865~regulation | 203665_AT, 203140_AT, 205891_AT, 221060_S_AT, 1552798_A_AT, 226218_AT, |
| of cell activation | 223501_AT, 223502_S_AT, 215990_S_AT, 205798_AT, 210895_S_AT, 222062_AT, |
| | 224341_X_AT, 204116_AT, 207433_AT, 202284_S_AT, 232068_S_AT |
| GO: 0048545~response | 203665_AT, 207542_S_AT, 203980_AT, 217757_AT, 1405_I_AT, 204655_AT, |
| to steroid hormone | 210145_AT, 238669_AT, 208712_AT, 207433_AT, 1555759_A_AT, 202827_S_AT, |
| stimulus | 202284_S_AT, 209047_AT, 215813_S_AT, 205128_X_AT |
| GO: 0006935~chemotaxis | 209906_AT, 207433_AT, 204655_AT, 1405_I_AT, 1555759_A_AT, 209392_AT, |
| | 221463_AT, 222877_AT, 235286_AT, 205898_AT, 205099_S_AT, 211661_X_AT |
| GO: 0042330~taxis | 209906_AT, 207433_AT, 204655_AT, 1405_I_AT, 1555759_A_AT, 209392_AT, |
| | 221463_AT, 222877_AT, 235286_AT, 205898_AT, 205099_S_AT, 211661_X_AT |
| GO: 0002703~regulation | 205798_AT, 203665_AT, 205891_AT, 203140_AT, 222062_AT, 207433_AT, |
| of leukocyte mediated | 220066_AT, 226218_AT, 215990_S_AT |
| immunity | |
| GO: 0002822~regulation | 205798_AT, 203140_AT, 210895_S_AT, 222062_AT, 207433_AT, 220066_AT, |
| of adaptive immune | 226218_AT, 223501_AT, 223502_S_AT, 215990_S_AT |
| response based on | |
| somatic recombination | |
| of immune receptors | |
| built from | |
| immunoglobulin | |
| superfamily domains | |
| G0: 0001568~blood | 229221_AT, 203665_AT, 201069_AT, 214297_AT, 212298_AT, 213503_X_AT, |
| vessel development | 212171_X_AT, 204575_S_AT, 208816_X_AT, 210510_S_AT, 239519_AT, |
| | 210427_X_AT, 210513_S_AT, 201590_X_AT, 222877_AT, 211962_S_AT, |
| | 202827_S_AT, 210512_S_AT, 211527_X_AT, 200878_AT |
| G0: 0030154~cell | 220333_AT, 210495_X_AT, 229221_AT, 212298_AT, 222877_AT, 205798_AT, |
| differentiation | 201278_AT, 211962_S_AT, 242871_AT, 203936_S_AT, 209348_S_AT, 230360_AT, |
| | 204141_AT, 200878_AT, 1569149_AT, 201279_S_AT, 204998_S_AT, 239519_AT, |
| | 201565_S_AT, 221266_S_AT, 233540_S_AT, 204999_S_AT, 226066_AT, 212190_AT, |
| | 215990_S_AT, 212143_S_AT, 220484_AT, 243556_AT, 210095_S_AT, 201324_AT, |
| | 212464_S_AT, 216442_X_AT, 212171_X_AT, 214297_AT, 204575_S_AT, 227240_AT, |
| | 201566_X_AT, 211719_X_AT, 228964_AT, 226218_AT, 207233_S_AT, 207433_AT, |
| | 210512_S_AT, 202827_S_AT, 205419_AT, 211527_X_AT, 204465_S_AT, |
| | 1554992_AT, 203140_AT, 1560960_AT, 201952_AT, 1569150_X_AT, 203980_AT, |
| | 220935_S_AT, 210510_S_AT, 210513_S_AT, 213931_AT, 204105_S_AT, 208712_AT, |
| | 210757_X_AT, 229797_AT, 201280_S_AT, 201951_AT |
| GO: 0001944~vasculature | 229221_AT, 203665_AT, 201069_AT, 214297_AT, 212298_AT, 213503_X_AT, |
| development | 212171_X_AT, 204575_S_AT, 208816_X_AT, 210510_S_AT, 239519_AT, |
| | 210427_X_AT, 210513_S_AT, 201590_X_AT, 222877_AT, 211962_S_AT, |
| | 202827_S_AT, 210512_S_AT, 211527_X_AT, 200878_AT |
| G0: 0002819~regulation | 205798_AT, 203140_AT, 210895_S_AT, 222062_AT, 207433_AT, 220066_AT, |
| of adaptive immune | 226218_AT, 223501_AT, 223502_S_AT, 215990_S_AT |
| response | |
| GO: 0051241~negative | 203665_AT, 203140_AT, 205891_AT, 213503_X_AT, 212171_X_AT, 208816_X_AT, |
| regulation of | 210427_X_AT, 210513_S_AT, 2101590_X_AT, 215990_S_AT, 206835_AT, 207433_AT, |
| multicellular organismal | 210512_S_AT, 220066_AT, 211527_X_AT |
| GO: 0051704~multi- | 237252_AT, 202727_S_AT, 1405_I_AT, 221060_S_AT, 205898_AT, 211661_X_AT, |
| organism process | 210895_S_AT, 222062_AT, 204116_AT, 232746_AT, 207433_AT, 208436_S_AT, |
| | 220066_AT, 232068_S_AT, 204475_AT, 242903_AT, 37145_AT, 214453_S_AT, |
| | 204655_AT, 1552553_A_AT, 211676_S_AT, 1552798_A_AT, 229937_X_AT, |
| | 210145_AT, 206157_AT, 203888_AT, 212977_AT, 224341_X_AT, 203887_S_AT, |
| | 225097_AT, 205495_S_AT, 1555756_A_AT, 1555759_A_AT, 202638_S_AT, |
| | 213293_S_AT |
| GO: 0007165~signal | 212298_AT, 209555_S_AT, 227948_AT, 223660_AT, 210360_S_AT, 219412_AT, |
| transduction | 222858_S_AT, 205798_AT, 206488_S_AT, 205960_AT, 206171_AT, 225173_AT, |
| | 220066_AT, 200878_AT, 237442_AT, 242903_AT, 225166_AT, 218589_AT, |
| | 203655_AT, 205891_AT, 207610_S_AT, 225171_AT, 209568_S_AT, 220307_AT, |
| | 239519_AT, 230266_AT, 209684_AT, 218856_AT, 1553982_A_AT, 223502_S_AT, |
| | 203104_AT, 212977_AT, 223939_AT, 224341_X_AT, 204834_AT, 206637_AT, |
| | 228766_AT, 1555756_A_AT, 230925_AT, 214581_X_AT, 214297_AT, 218501_AT, |
| | 202727_S_AT, 1405_I_AT, 228708_AT, 221060_S_AT, 202609_AT, 226218_AT, |
| | 223501_AT, 205898_AT, 205099_S_AT, 211661_X_AT, 214841_AT, 204116_AT, |
| | 232746_AT, 225207_AT, 207433_AT, 227265_AT, 225188_AT, 221463_AT, |
| | 205419_AT, 232068_S_AT, 209906_AT, 228368_AT, 1554992_AT, 201952_AT, |
| | 210510_S_AT, 204655_AT, 225189_S_AT, 230559_X_AT, 214054_AT, 203037_S_AT, |
| | 210264_AT, 211676_S_AT, 1552798_A_AT, 216250_S_AT, 237904_AT, 208712_AT, |
| | 225097_AT, 219994_AT, 1555759_A_AT, 241929_AT, 219434_AT, 201951_AT |
| GO: 0009617~response | 237252_AT, 1405_I_AT, 204655_AT, 1552553_A_AT, 221060_S_AT, 1552798_A_AT, |
| to bacterium | 210145_AT, 203888_AT, 211661_X_AT, 222062_AT, 224341_X_AT, 203887_S_AT, |
| | 207433_AT, 205495_S_AT, 1555759_A_AT, 220066_AT, 232068_S_AT, 37145_AT |
| GO: 0048519~negative | 213891_S_AT, 1553995_A_AT, 212298_AT, 224218_S_AT, 205798_AT, 201278_AT, |
| regulation of biological | 228057_AT, 202284_S_AT, 220066_AT, 203939_AT, 228490_AT, 203665_AT, |
| process | 205891_AT, 201279_S_AT, 204998_S_AT, 208816_X_AT, 201565_S_AT, 239519_AT, |
| | 203753_AT, 201590_X_AT, 204999_S_AT, 226066_AT, 223502_S_AT, 215990_S_AT, |
| | 221815_AT, 225337_AT, 212143_S_AT, 224341_X_AT, 201422_AT, 222146_S_AT, |
| | 222651_S_AT, 229435_AT, 201005_AT, 215813_S_AT, 210095_S_AT, 205681_AT, |
| | 205128_X_AT, 214211_AT, 1560485_AT, 212171_X_AT, 213503_X_AT, 63825_AT, |
| | 1405_I_AT, 221060_S_AT, 210427_X_AT, 201566_X_AT, 228964_AT, 226218_AT, |
| | 223501_AT, 212387_AT, 223398_AT, 244579_AT, 218559_S_AT, 205566_AT, |
| | 207233_S_AT, 222062_AT, 218502_S_AT, 206835_AT, 207433_AT, 210512_S_AT, |
| | 202827_S_AT, 222670_S_AT, 208436_S_AT, 232068_S_AT, 87100_AT, |
| | 211527_X_AT, 212382_AT, 203140_AT, 203980_AT, 217757_AT, 204655_AT, |
| | 210510_S_AT, 210513_S_AT, 1552798_A_AT, 206675_S_AT, 213931_AT, |
| | 238669_AT, 212614_AT, 208712_AT, 210258_AT, 210757_X_AT, 212386_AT, |
| | 206134_AT, 225097_AT, 1555759_A_AT, 1556423_AT, 201280_S_AT, 223394_AT |
| GO: 0050864~regulation | 203140_AT, 222062_AT, 204116_AT, 207433_AT, 202284_S_AT, 223501_AT, |
| of B cell activation | 223502_S_AT, 215990_S_AT |
| GO: 0001525~angiogenesis | 203665_AT, 214297_AT, 213503_X_AT, 212171_X_AT, 212298_AT, 204575_S_AT, |
| | 208816_X_AT, 210510_S_AT, 210427_X_AT, 210513_S_AT, 239519_AT, |
| | 201590_X_AT, 222877_AT, 202827_S_AT, 210512_S_AT, 211527_X_AT, 200878_AT |
| GO: 0048869~cellular | 220333_AT, 210495_X_AT, 229221_AT, 212298_AT, 222877_AT, 205798_AT, |
| developments process | 201278_AT, 211962_S_AT, 242871_AT, 203936_S_AT, 209348_S_AT, 230360_AT, |
| | 204141_AT, 200878_AT, 1569149_AT, 201279_S_AT, 204998_S_AT, 239519_AT, |
| | 201565_S_AT, 221266_S_AT, 233540_S_AT, 204999_S_AT, 226066_AT, 212190_AT, |
| | 215990_S_AT, 212143_S_AT, 220484_AT, 201005_AT, 243556_AT, 210095_S_AT, |
| | 201324_AT, 212464_S_AT, 216442_X_AT, 212171_X_AT, 214297_AT, 204575_S_AT, |
| | 227240_AT, 201566_X_AT, 211719_X_AT, 228964_AT, 226218_AT, 207233_S_AT, |
| | 207433_AT, 210512_S_AT, 202827_S_AT, 205419_AT, 211527_X_AT, 204465_S_AT, |
| | 1554992_AT, 203140_AT, 1560960_AT, 201952_AT, 1569150_X_AT, 203980_AT, |
| | 220935_S_AT, 210510_S_AT, 210513_S_AT, 213931_AT, 204105_S_AT, 208712_AT, |
| | 210757_X_AT, 229797_AT, 201280_S_AT, 201951_AT |
| GO: 0002694~regulation | 203140_AT, 205891_AT, 203665_AT, 226218_AT, 223501_AT, 215990_S_AT, |
| of leukocyte activation | 223502_S_AT, 205798_AT, 210895_S_AT, 222062_AT, 204116_AT, 207433_AT, |
| | 202284_S_AT |
| GO: 0044243~multicellular | 201069_AT, 204575_S_AT, 203936_S_AT, 204475_AT |
| organismal catabolic | |
| process | |
| GO: 0030574~collagen | 201069_AT, 204575_S_AT, 203936_S_AT, 204475_AT |
| cataboltc process | |
| GO: 0016477~cell | 216442_X_AT, 210495_X_AT, 229221_AT, 1560960_AT, 212298_AT, 210510_S_AT, |
| migration | 1405_I_AT, 204655_AT, 239519_AT, 211719_X_AT, 222877_AT, 204268_AT, |
| | 204105_S_AT, 212614_AT, 207433_AT, 202827_S_AT, 1555759_A_AT, 235286_AT, |
| | 202638_S_AT, 212464_S_AT |
| GO: 0045595~regulation | 212298_AT, 209555_S_AT, 1405_I_AT, 201566_X_AT, 221040_S_AT, 226218_AT |
| of cell differentiation | 218559_S_AT, 205798_AT, 207233_S_AT, 206488_S_AT, 210895_S_AT, 204116_AT, |
| | 209348_S_AT, 222670_S_AT, 232068_S_AT, 1569149_AT, 203140_AT, |
| | 1569150_X_AT, 204998_S_AT, 220935_S_AT, 210510_S_AT, 204655_AT, |
| | 239519_AT, 201565_S_AT, 1552798_A_AT, 233540_S_AT, 204999_S_AT, |
| | 226066_AT, 215990_S_AT, 213931_AT, 212143_S_AT, 204105_S_AT, 208712_AT, |
| | 224341_X_AT, 228766_AT, 1555759_A_AT, 241929_AT, 210095_S_AT |
| GO: 0032879~regulation | 212298_AT, 212171_X_AT, 209555_S_AT, 63825_AT, 228708_AT, 205566_AT, |
| of localization | 206488_S_AT, 207433_AT, 203936_S_AT, 210512_S_AT, 87100_AT, 220066_AT, |
| | 211527_X_AT, 228490_AT, 203665_AT, 203140_AT, 205891_AT, 210510_S_AT, |
| | 239519_AT, 210513_S_AT, 212190_AT, 210145_AT, 221815_AT, 215990_S_AT, |
| | 206157_AT, 212143_S_AT, 225337_AT, 238669_AT, 228766_AT, 1555756_A_AT, |
| | 209392_AT, 1556423_AT, 241929_AT, 210095_S_AT, 215813_S_AT, 202638_S_AT, |
| | 205128_X_AT |
| GO: 0050729~positive | 205891_AT, 224341_X_AT, 203980_AT, 204655_AT, 1405_I_AT, 221060_S_AT, |
| regulation of | 1555759_A_AT, 232068_S_AT, 1552798_A_AT, 210145_AT |
| inflammatory response | |
| GO: 0007186~G-protein | 218589_AT, 209906_AT, 205891_AT, 207610_S_AT, 223660_AT, 1405_I_AT, |
| coupled receptor protein | 204655_AT, 210264_AT, 205898_AT, 205099_S_AT, 237904_AT, 211661_X_AT, |
| signaling pathway | 212977_AT, 210258_AT, 232746_AT, 223939_AT, 206171_AT, 206637_AT, |
| | 1555759_A_AT, 209392_AT, 205419_AT |
| GO: 0051270~regulation | 228490_AT, 203665_AT, 203140_AT, 212298_AT, 212171_X_AT, 63825_AT, |
| of cell motion | 210510_S_AT, 239519_AT, 210513_S_AT, 212190_AT, 215990_S_AT, 221815_AT, |
| | 212143_S_AT, 205566_AT, 225337_AT, 203936_S_AT, 210512_S_AT, 209392_AT, |
| | 1556423_AT, 87100_AT, 211527_X_AT, 210095_S_AT, 202638_S_AT |
| GO: 0048518~positive | 213891_S_AT, 229221_AT, 212298_AT, 209555_S_AT, 227948_AT, 222838_AT, |
| regulation of biological | 242794_AT, 205798_AT, 206488_S_AT, 217999_S_AT, 217997_AT, 209348_S_AT, |
| process | 203936_S_AT, 202284_S_AT, 220066_AT, 232333_AT, 200878_AT, 1569149_AT, |
| | 205891_AT, 203665_AT, 201565_S_AT, 239519_AT, 203753_AT, 234645_AT, |
| | 210145_AT, 226066_AT, 215990_S_AT, 206157_AT, 223502_S_AT, 212143_S_AT, |
| | 215602_AT, 224341_X_AT, 222146_S_AT, 228766_AT, 1555756_A_AT, 243556_AT, |
| | 229435_AT, 215813_S_AT, 210095_S_AT, 205128_X_AT, 212171_X_AT, 218501_AT, |
| | 1553906_S_AT, 227240_AT, 1405_I_AT, 201566_X_AT, 235457_AT, 221060_S_AT, |
| | 228964_AT, 226218_AT, 223501_AT, 212387_AT, 218559_S_AT, 207233_S_AT, |
| | 210895_S_AT, 222062_AT, 204116_AT, 207433_AT, 222670_S_AT, 210512_S_AT, |
| | 232068_S_AT, 208436_S_AT, 211527_X_AT, 244414_AT, 212382_AT, 209906_AT, |
| | 1554992_AT, 203140_AT, 1569150_X_AT, 242405_AT, 203980_AT, 210510_S_AT, |
| | 204655_AT, 1552553_A_AT, 230559_X_AT, 210513_S_AT, 1565754_X_AT, |
| | 1552798_A_AT, 208978_AT, 225842_AT, 213931_AT, 238669_AT, 1565752_AT, |
| | 204105_S_AT, 208712_AT, 212386_AT, 225097_AT, 1555759_A_AT, 241929_AT, |
| | 202638_S_AT, 223394_AT |
| GO: 001816~cytokine | 224341_X_AT, 203980_AT, 1552553_A_AT, 209348_S_AT, 221060_S_AT, |
| production | 220066_AT, 232068_S_AT, 1552798_A_AT, 211661_X_AT |
| GO: 0007610~behavior | 209906_AT, 1554992_AT, 1405_I_AT, 204655_AT, 202609_AT, 222877_AT, |
| | 205898_AT, 205099_S_AT, 211661_X_AT, 220484_AT, 225097_AT, 207433_AT, |
| | 229797_AT, 223092_AT, 1555759_A_AT, 209392_AT, 221463_AT, 235286_AT |
| GO: 00550878~regulation | 237252_AT, 213503_X_AT, 209555_S_AT, 208816_X_AT, 210427_X_AT, |
| of body fluid levels | 201590_X_AT, 203888_AT, 206488_S_AT, 206835_AT, 203887_S_AT, 228766_AT, |
| | 235944_AT, 241929_AT, 201005_AT, 203060_S_AT |
| GO: 0032496~response | 237252_AT, 1405_I_AT, 204655_AT, 221060_S_AT, 1552798_A_AT, 210145_AT, |
| to lipopolysaccharide | 203888_AT, 211661_X_AT, 224341_X_AT, 203887_S_AT, 207433_AT, |
| | 1555759_A_AT, 232068_S_AT, 220066_AT |
| GO: 0002683~negative | 205798_AT, 203665_AT, 203140_AT, 222062_AT, 217757_AT, 207433_AT, |
| regulation of immune | 220066_AT, 226218_AT, 215990_S_AT |
| GO: 0051674~localization | 216442_X_AT, 210495_X_AT, 229221_AT, 1560960_AT, 212298_AT, 210510_S_AT, |
| of cell | 1405_I_AT, 204655_AT, 239519_AT, 211719_X_AT, 222877_AT, 204268_AT, |
| | 204105_S_AT, 212614_AT, 207433_AT, 202827_S_AT, 1555759_A_AT, 235286_AT, |
| | 202638_S_AT, 212464_S_AT |
| GO: 0048870~cell motility | 216442_X_AT, 210495_X_AT, 229221_AT, 1560960_AT, 212298_AT, 210510_S_AT, |
| | 1405_I_AT, 204655_AT, 239519_AT, 211719_X_AT, 222877_AT, 204268_AT, |
| | 204105_S_AT, 212614_AT, 207433_AT, 202827_S_AT, 1555759_A_AT, 235286_AT, |
| | 202638_S_AT, 212464_S_AT |
| GO: 0046903~secretion | 203665_AT, 205891_AT, 207542_S_AT, 213503_X_AT, 201279_S_AT, 208816_X_AT, |
| | 1405_I_AT, 204655_AT, 210427_X_AT, 1552553_A_AT, 201590_X_AT, 227134_AT, |
| | 223501_AT, 223502_S_AT, 201278_AT, 206835_AT, 210757_X_AT, 1555759_A_AT, |
| | 209047_AT, 201280_S_AT, 235286_AT |
| GO: 0006928~cell motion | 210495_X_AT, 216442_X_AT, 229221_AT, 212298_AT, 1405_I_AT, 211719_X_AT, |
| | 210360_S_AT, 222877_AT, 204268_AT, 207433_AT, 202827_S_AT, 235286_AT, |
| | 1560960_AT, 201952_AT, 204655_AT, 210510_S_AT, 239519_AT, 203037_S_AT, |
| | 212614_AT, 204105_S_AT, 1555759_A_AT, 209392_AT, 201005_AT, 201951_AT, |
| | 202638_S_AT, 212464_S_AT |
| GO: 0048514~blood | 203665_AT, 214297_AT, 213503_X_AT, 212171_X_AT, 212298_AT, 204575_S_AT, |
| vessel morphogenesis | 208816_X_AT, 210510_S_AT, 239519_AT, 210427_X_AT, 210513_S_AT, |
| | 201590_X_AT, 222877_AT, 211962_S_AT, 202827_S_AT, 210512_S_AT, |
| | 211527_X_AT, 200878_AT |
| GO: 0001501~skeletal | 201069_AT, 213503_X_AT, 1569150_X_AT, 210427_X_AT, 201590_X_AT, |
| system development | 224218_S_AT, 204653_AT, 244579_AT, 212143_S_AT, 212614_AT, 206835_AT, |
| | 218502_S_AT, 203936_S_AT, 202827_S_AT, 223092_AT, 222651_S_AT, |
| | 203060_S_AT, 210095_S_AT, 1569149_AT |
| GO: 0050867~positive | 203140_AT, 205891_AT, 221060_S_AT, 1552798_A_AT, 226218_AT, 223501_AT, |
| regulation of cell | 223502_S_AT, 215990_S_AT, 205798_AT, 210895_S_AT, 224341_X_AT, 204116_AT, |
| activation | 202284_S_AT, 232068_S_AT |
| GO: 0030247~polysaccharide | 229221_AT, 216442_X_AT, 210495_X_AT, 212171_X_AT, 210513_S_AT, |
| binding | 211719_X_AT, 212190_AT, 206157_AT, 1555756_A_AT, 210512_S_AT, 220066_AT, |
| | 209392_AT, 211527_X_AT, 212464_S_AT |
| GO: 0001871~pattern | 229221_AT, 216442_X_AT, 210495_X_AT, 212171_X_AT, 210513_S_AT, |
| binding | 211719_X_AT, 212190_AT, 206157_AT, 1555756_A_AT, 210512_S_AT, 220066_AT, |
| | 209392_AT, 211527_X_AT, 212464_S_AT |
| GO: 0004222~metalloendopeptidase | 201069_AT, 206134_AT, 204575_S_AT, 229004_AT, 203936_S_AT, 202827_S_AT, |
| activity | 204475_AT |
| GO: 0002698~negative | 205798_AT, 203665_AT, 203140_AT, 217757_AT, 226218_AT, 215990_S_AT |
| regulation of immune | |
| effector process | |
| GO: 0002698~positive | 205891_AT, 212171_X_AT, 203980_AT, 1405_I_AT, 204655_AT, 221060_S_AT, |
| regulation of response to | 210513_S_AT, 1552798_A_AT, 210145_AT, 224341_X_AT, 1555759_A_AT, |
| external stimulus | 210512_S_AT, 232068_S_AT, 211527_X_AT |
| GO: 0010033~response | 237252_AT, 229221_AT, 202435_S_AT, 1405_I_AT, 201566_X_AT, 221060_S_AT, |
| to organic substance | 202609_AT, 218934_S_AT, 204058_AT, 211661_X_AT, 202434_S_AT, 207433_AT, |
| | 202827_S_AT, 202284_S_AT, 209047_AT, 220066_AT, 232068_S_AT, 203665_AT, |
| | 207542_S_AT, 202436_S_AT, 217757_AT, 204059_S_AT, 203980_AT, 204655_AT, |
| | 202437_S_AT, 201565_S_AT, 1552798_A_AT, 210145_AT, 213931_AT, 203888_AT, |
| | 238669_AT, 208712_AT, 203887_S_AT, 224341_X_AT, 1555756_A_AT, |
| | 1555759_A_AT, 215813_S_AT, 209122_AT, 205128_X_AT |

| Term | Genes |
|---|---|
| Enriched among top downregulated genes: | |
| GO: 0007275~multicellular | BMP8B, TEAD4, FLOT2, IRX3, ADAMTS1, DZIP1, CSPGS, |
| organismal | COL8A2, EBF3, TSPAN2, FKBP4, HK2P1, CXCR4, GFI1, |
| development | FLOT2, SIK1, DMRT2, TPDS2, TPDS2, WT1, TPDS2, |
| | WNT3, LGR4, SLC1A3, BCL11A, BCL2, SPRY1, PCDH8, |
| | CNTN4, BCL11A, ECE2, BMP8B, CXCR4, CAV1, WT1, |
| | KCNQ4, POU4F2, CXCR4, KAZ, OSR2, RET, COL8A2, |
| | BCL11A, BCL2, PKP2 |
| GO: 0048731~system | BMP8B, TEAD4, FLOT2, IRX3, ADAMTS1, CSPG5, |
| development | COLBA2, TSPAN2, HK2P1, CXCR4, GFI1, FLOT2, DMRT2, |
| | TPD52, TPD52, WT1, TPD52, WNT3, LGR4, SLC1A3, |
| | BCL2, BCL11A, SPRY1, PCDH8, CHTN4, BCL11A, ECE2, |
| | BMP8B, CXCR4, WT1, CAV1, KCNQ4, POU4F2, CXCR4, |
| | KAZ, OSR2, RET, COL8A2, BCL11A, BCL2, PKP2 |
| GO: 0048513~organ | BMP8B, TEAD4, FLOT2, ADAMTS1, COLBA2, HK2P1, |
| development | CXCR4, GFI1, FLOT2, DMRT2, TPD52, TPD52, WT1, |
| | TPD52, WNT3, LGR4, BCL2, BCL11A, SPRY1, PCDH8, |
| | CMTN4, BCL11A, ECE2, BMP8B, CXCR4, WT1, CAV1, |
| | KCNQ4, POU4F2, CXCR4, KAZ, RET, OSR2, COL8A2, BCL2, |
| | BCL11A, PKP2 |
| GO: 0048856~anatomical | BMP8B, TEAD4, FLOT2, IRX3, ADAMTS1, CSPG5, |
| structure development | COL8A2, TSPAN2, HK2P1, CXCR4, GFI1, FLOT2, DMRT2, |
| | TPD52, TPD52, WT1, TPD52, WNT3, LGR4, SLC1A3, |
| | BCL2, BCL11A, SPRY1, PCDH8, CNTN4, BCL11A, ECE2, |
| | BMP8B, CXCR4, WT1, CAV1, KCNQ4, POU4F2, CXCR4, |
| | KAZ, OSR2, RET, COL8A2, BCL11A, BCL2, PKP2 |
| GO: 0030154~cell | BMP8B, TDRD7, TEAD4, IRX3, DZIP1, CSPG5, TSPAN2, |
| differentiation | CXCR4, GFI1, SIK1, TPO52, TPD52, WT1, TPD52, WNT3, |
| | SLC1A3, BCL11A, BCL2, SPRY1, CNTN4, BCL11A, ECE2, |
| | BMP8B, CAV1, WT2, CXCR4, POU4F2, CXCR4, KAZ, RET, |
| | BCX11A, BCL2 |
| GO: 0032502~developmental | BMP8B, TDRD7, TEAD4, FLOT2, IRX3, ADAMTS1, DZIP1, |
| process | CSPG5, COLBA2, EBF3, TSPAN2, FKBP4, HK2P1, CXCR4, |
| | GFI1, FLOT2, SIK1, DMRT2, TPD52, TPD52, WT1, TPD52, |
| | WNT3, LGR4, SLC1A3, BCL11A, BCL2, SPRY1, PCDH8, |
| | CNTN4, BCL11A, ECE2, BMP8B, CXCR4, CAV1, WT1, |
| | KCNQ4, POU4F2, CXCR4, KAZ, OSR2, RET, COLBA2, |
| | BCL11A, BCL2, PKP2 |
| GO: 0003006~reproductive | TDRD7, LGR4, BCL2, AQAMTS1, DZIP1, WT1, CXCR4, |
| developmental | FKBP4, CXCR4, CXCR4, BCL2, DMRT2, WT1 |
| process | |
| GO: 0048869~cellular | BMP8B, TDRD7, TEAD4, IRX3, DZIP1, CSPG5, TSPAN2, |
| developmental process | CXCR4, GFI1, SIK1, TPD52, TPD52, WT1, TPD52, WNT3, |
| | SLC1A3, BCL11A, BCL2, SPRY1, CNTN4, BCL11A, ECE2, |
| | BMP8B, CAV1, WT1, CXCR4, POU4F2, CXCR4, KAZ, RET, |
| | BCL11A, BCL2 |
| GO: 0009653~anatomical | TEAD4, ADAMTS1, CQLBA2, CXCR4, GFI1, TPD52, TPD52, |
| structure morphogenesis | WT1, TPD52, WNT3, LGR4, SLC1A3, BCL2, CNTN4, |
| | PCDH8, WT1, CXCR4, CAV1, KCNQ4, POU4F2, OSR2, |
| | RET, CXCR4, COL8A2, BCL2 |
| Enriched among top upregulated genes: | |
| GO: 0005886~plasma | ADAP2, NT5E, FN1, CD44, MFI2, NRP1, CD36, ADORA3, |
| membrane | PCDHGA12, RAB3S, IL7R. KCNQ3, GLDN, CD109, |
| | IFNGR1, EMR2, CD244, SLC43A2, ANXA2P1, CYBB, |
| | LILRB1, SLC38A6, CSF1R, CXCR7, GPC1, CD36, APBB1IP, |
| | NGEF, CD9, ATP1B1, GBP2, PLIN2, PTGS1, FTHL3, THBD, |
| | CSPG4, IFNGR1, TLR4, FN1, SLC12A6, TNFSF13B, |
| | CX3CR1, CD52, CCR1, PSD3, LILRB2, PCDHGA12, |
| | SLC7A11, TLR4, AQP1, GBP5, RASGRF1, C3AR1, TRPM8, |
| | NRP1, SLC6A6, IFNGR1, PTPRO, PTGS1, NRCAM, |
| | APBB1IP, ITGB5, PSD3, ICAM1, ATP1B1, IL10RA, MMP2, |
| | CD180, SLC43A2, NRP2, SYTL1, ST3GAL5, CD109, EVL, |
| | CD36, ADORA3, PTRF, ATP6V0D2, NOD2, PCDH7, |
| | APBB1IP, NT5E, STEAP1, LPAR6, SLC41A2, COL6A1, |
| | LOC100131909, HMOX1, AQP1, GBP2, ANXA2P2, NRP1, |
| | ABCC3, TM7SF4, SLC7A11, TNFSF13B, PTRF, LMNA, |
| | MCOLN3, THBD, TLR4, SUCNR1, P2RY14, CLEC7A, |
| | ENPP2, ABCB1, PTGS1, EMP1, FN1, FN1, ANXA2P1, |
| | NGEF, ANXA2P1, EPS8, DIXDC1, IL7R, CD52, PTAFR, |
| | CD86, IL27RA, IL2RG, CXCR7, ANKH, RAPH1, MMP14, |
| | GPR183, SLC41A2, MDGA1, ALCAM, RAPH1, TLR4, |
| | GPR35, ITG87, ADORA3, THBD, SLC41A2, MTUS1, |
| | RGS13, OLR1, ADAP2, MCOLN3, PLEKHO1, CD36, |
| | TREM1, ALCAM |
| GO: 0031226~intrinsic to | MFI2, CD44, CD36, ADORA3, ST3GAL5, CD36, ADORA3, |
| plasma membrane | KCNQ3, PCDH7, IFNGR1, STEAP1, LOC100131909, AQP1, |
| | ABCC3, CYBB, CSF1R, GPC1, TLR4, THBD, CD36, ENPP2, |
| | CD9, ATP1B1, THBD, CSPG4, IFNGR1, TLR4, EPS8, |
| | SLC12A6, CD52, CD52, CX3CR1, CCR1, PTAFR, IL27RA, |
| | IL2RG, LILRB2, MMP14, ANKH, AQP1, GPR183, TLR4, |
| | C3AR1, MDGA1, SLC6A6, GPR35, IFNGR1, PTPRO, TLR4, |
| | ITG87, ADORA3, THBD, NRCAM, OLR1, CD36, ITG85, |
| | ICAM1, ATP1B1 |
| GO: 0016020~membrane | NT5E, MFI2, NRP1, ADORA3, SLAMF7, RAB38, IL7R, |
| | PDK4, KCNQ3, GDPD1, PHLDA1, CD109, MPZL3, GCNT1, |
| | CD244, SLC43A2, FADS3, ANXA2P1, CYBB, RHBDF1, |
| | ABHD2, CSF1R, GPC1, CD36, PTGS1, FTHL3, TNFRSF21, |
| | ABHD2, FN1, TNFSF13B, CX3CR1, CCR1, MARCH1, |
| | LILRB2, PCDHGA12, HMCN1, AQP1, GCNT1, RASGRF1, |
| | POPDC3, HAVCR2, IFNGR1, PHLDA1, PTGS1, FADS3, |
| | NRCAM, C4ORF34, ADAMDEC1, ICAM1, GCNT1, |
| | ATP1B1, IL10RA, SLC37A2, SLC43A2, SYTL1, NRP2, |
| | ATP9A, CYP1B1, DAPP1, GUPR1, CD109, TMEM39A, |
| | CD36, PTRF, ATP6V0D2, NOD2, PCDH7, STEAP1, |
| | TMEM158, HMOX1, LOC100131909, GBP2, ANXA2P2, |
| | NRP1, ABCC3, TNFRSF21, PLA2G4A, TNFSF13B, LPCAT2, |
| | TBXAS1, PTRF, DOCK4, LMNA, SUCNR1, THBD, P2RY14, |
| | KMO, CLEC7A, PTGS1, NGEF, ANXA2P1, IL7R, CD52, |
| | PARM1, CD86, IL27RA, CXCR7, VEGFA, RAPH1, VEGFA, |
| | C4ORF34, MDGA1, ALCAM, RAPH1, GPR35, ITG87, |
| | RGS13, DAB2, ADAP2, OLR1, MCOLN3, DAB2, ALCAM, |
| | PAQR5, ADAP2, FN1, CD44, CD36, CYP1B1, PCDHGA12, |
| | PHLDA1, AGPAT9, PAQR5, ATP10A, GLDN, IFNGR1, |
| | ABHD2, EMR2, LILRB1, SLC38A6, C40RF34, CXCR7, |
| | APBB1IP, NGEF, C09, ATP1B1, GBP2, PLIN2, THBD, |
| | VEGFA, CSPG4, ACPP, IFNGR1, TLR4, SLC12A6, CD52, |
| | ABHD2, PSD3, SLC7A11, TLR4, GBP5, CALHM2, C3AR1, |
| | TRPM8, NRP1, VEGFA, SLC6A6, PTPRO, GBP3, UGCG, |
| | HIPK2, APBB1IP, KMO, PSD3, ITGB5, GLIPR1, MMP2, |
| | CD180, ST3GAL5, DAB2, EVL, ADORA3, DSE, APBB1IP, |
| | NT5E, LPAR6, SLC41A2, COL6A1, CYP1B1, AQP1, |
| | CALHM2, DAB2, SLAMF8, TM7SF4, SLC7A11, ABHD2, |
| | MCOLN3, TLR4, LFNG, ENPP2, EMP1, ABCB1, FN1, |
| | GDPD1, FN1, ANXA2P1, RAB278, EPS8, DIXDC1, PTAFR, |
| | CNIH3, IL2RG, PDK4, SLAMF8, MMP14, ANKH, ABHD2, |
| | GPR183, CKLF, SLC41A2, HAVCR2, CYP1B1, TLR4, THBD, |
| | ADORA3, SLC41A2, MTUS1, PLEKHO1, CD36, TREM1 |
| GO: 0005887~integral to | MFI2, CD44, CD36, ADORA3, ST36AL5, CD36, ADORA3, |
| plasma membrane | KCNQ3, PCDH7, IFNGR1, STEAP1, LOC10Q131909, AQP1, |
| | ABCC3, CYBB, CSF1R, GFC1, TLR4, THBD, CD36, ENPP2, |
| | CD9, ATP181, THBD, CSPG4, IFNGR1, TLR4, EPS8, |
| | SLC12A6, CD52, CD52, CX3CR1, CCR1, PTAFR, IL27RA, |
| | IL2RG, LILRB2, MMP14, ANKH, AQP1, GPH183, TLR4, |
| | C3AR1, SLC6A6, GPR35, IFNGR1, PTPRO, TLR4, ITG87, |
| | ADORA3, THBD, NRCAM, OLR1, CD36, ITG85, ICAM1, |
| | ATP181 |
| GO: 0044459~plasma | FN1, MFI2, CD44, CD36, ADORA3, SVTL1, ST3GAL5, |
| membrane part | RAB38, IL7R, CD36, EVL, ADORA3, KCNQ3, PTRF, |
| | ATP6VOD2, PCDH7, AP881IP, IFNGR1, STEAP1, HMOX1, |
| | LOC100131909, AQP1, G8P2, CD244, ABCC3, CYBB, |
| | SLC38A6, PTRF, CSF1R, GPC1, TLR4, THBD, CD36, |
| | CLEC7A, APBB1IP, EWPP2, CD9, ATP1B1, ABCB1, GBF2, |
| | FN1, THBO, FN1, CSPG4, IFNGR1, TLR4, FN1, EPS8, |
| | DIXDC1, SLC12A6, IL7R, CD52, CD52, CX3CR1, CCR1, |
| | PTAFR, CD86, PSD3, IL27RA, IL2RG, LILRB2, MMP14, |
| | ANKH, RAPH1, AQP1, GPR183, TLR4, GBP5, C3AR1, |
| | MDGA1, TRPM8, ALCAM, RAPH1, SLC6A6, GPR35, |
| | IFNGR1, PTPRO, TLR4, ITGB7, ADORA3, THBD, NRCAM, |
| | OLR1, APBB1IP, CD36, PSD3, ITG85, ALCAM, ICAM1, |
| | ATP1B1 |
| GO: 0005576~extracellular | GLIPR1, CD44, MFI2, FN1, MMP2, NRP1, IL7R, GLIPR1, |
| region | CD109, MMP9, CD109, GNLY, HMOX1, COL6A1, |
| | ANXA2P2, NRP1, ANXA2P1, SERPINE2, TNFSF13B, PTX3, |
| | IGFBP3, PRSS23, THBD, GPC1, IFI30, FGL2, GNLY, |
| | COL22A1, ENPP2, LFNG, IGFBP3, PGA5, PLIN2, FN1, |
| | THBD, FN1, ACPP, VEGFA, ANXA2P1, MMP19, CCL5, |
| | ANXA2P1, FN1, IL7R, TNFSF13B, STATH, IL10, FGL2, |
| | HMCN1, VEGFA, MMP14, CCL24, VEGFA, MMP1, CKLF, |
| | A2M, ADAMTS15, CCL5, NRP1, VEGFA, TH8D, OLR1, |
| | ADAMDEC1, CTSL1, CCL5, VASH1, TREM1, ICAM1 |
| GO: 0031224~intrinsic to | PAQR5, NT5E, CD44, MFI2, NRP1, CD36, ADORA3, |
| membrane | SLAMF7, PCDHGA12, IL7R, AGPAT9, ATP10A, PAQR5, |
| | GDPD1, KCNQ3, GLDN, CD109, IFNGR1, MPZL3, ABHD2, |
| | EMR2, GCNT1, CD244, SLC43A2, FADS3, CTBB, RHBDF1, |
| | LILRB1, ABHD2, SLC38A6, C4ORF34, CSF1R, CXCR7, |
| | GPC1, CD36, CD9, ATP1B1, TNFRSF21, THBD, ACPP, |
| | CSPG4, ABHD2, IFNGR1, TLR4, SLC12A6, TNFSF13B, |
| | CX3CR1, CD52, CCR1, ABHD2, MARCH1, LILRB2, |
| | PCDHGA12, SLC7A11, TLR4, GCNT1, AQP1, CALHM2, |
| | C3AR1, TRPM8, NRP1, SLC6A6, HAVCR2, POPDC3, |
| | IFNGR1, PTPRO, FADS3, GBP3, C4ORF34, NRCAM, |
| | UGCG, ADAMDEC1, KMO, ITG85, ICAM1, GCNT1, |
| | ATP1B1, GLIPR1, IL10RA, SLC37A2, CD180, SLC43A2, |
| | NRP2, ST3GAL5, ATP9A, CD109, GLIPR1, CD36, |
| | TMEM39A, ADORA3, DSE, PCDH7, NT5E, STEAP1, |
| | TMEM15B, LPAR6, SLC41A2, LOC100131909, AQP1, |
| | CALHM2, NRP1, SLAMF8, ABCC3, TM7SF4, SLC7A11, |
| | TNFRSF21, PLA2G4A, LPCAT2, TNFSF13B, ABHD2, |
| | TBXAS1, MCOLN3, THBD, TLR4, SUCNR1, P2RY14, KMO, |
| | CLEC7A, ENPP2, LFNG, ABCB1, EMP1, GDPD1, RAB27B, |
| | EPS8, JL7R, CD52, PARM1, PTAFR, CNIH3, CD86, IL27RA, |
| | CXCR7, IL2RG, SLAMF8, ANKH, MMP14, GPR183, |
| | ABHD2, CKLF, C4ORF34, SLC41A2, MDGA1, HAVCR2, |
| | ALCAM, TLR4, GPR35, ITGB7, ADORA3, THBD, SLC41A2, |
| | OLR1, MCOLN3, CD36, TREM1, ALCAM |
| GO: 0004871~signal | PAQR5, IL10RA, CD44, NRP1, CD36, ADORA3, CD180, |
| transducer activity | SLAMF7, NRP2, IL7R, CD36, PDK4, ADORA3, PAQR5, |
| | EPAS1, IFNGR1, LPAR6, SLA, HMOX1, LOC100131909, |
| | EMR2, CD244, NRP1, TNFRSF21, RHBDF1, LILRB1, |
| | CSF1R, CXCR7, SUCNR1, TLR4, THBD, P2RY14, CD36, |
| | CLEC7A, ENPP2, TNFRSF21, THBD, CSPG4, IFNGR1, CCL5, |
| | TLR4, EPS8, DIXDC1, IL7R, CX3CR1, CCR1, PTAFR, CD86, |
| | IL27RA, IL2RG, CXCR7, PDK4, LILRB2, HMCN1, GPR183, |
| | TLR4, C3AR1, TRPM8, NRP1, CCL5, DOK2, IFNGR1, |
| | PTPRO, TLR4, GPR35, SKIL, ITG87, THBD, ADORA3, SLA, |
| | RGS13, OLR1, CCL5, CD36, ITGB5, TREM1, ICAM1 |
| GO: 0060089~molecular | PAQR5, IL10RA, CD44, NRP1, CD36, ADORA3, CD180, |
| transducer activity | SLAMF7, NRP2, IL7R, CD36, PDK4, ADORA3, PAQR5, |
| | EPAS1, IFNGR1, LPAR6, SLA, HMOX1, LOC100131909, |
| | EMR2, CD244, NRP1, TNFRSF21, RHBDF1, LILRB1, |
| | CSF1R, CXCR7, SUCNR1, TLR4, TH8D, P2RY14, CD36, |
| | CLEC7A, ENPP2, TNFRSF21, THBD, CSPG4, IFNGR1, CCL5, |
| | TLR4, EPS8, DIXDC1, IL7R, CX3CR1, CCR1, PTAFR, CD86, |
| | IL27RA, IL2RG, CXCR7, PDK4, ULRB2, HMCN1, GPR183, |
| | TLR4, C3AR1, TRPM8, NRP1, CCL5, DOK2, IFNGR1, |
| | PTPRO, TLR4, GPR35, SKIL, ITGB7, THBD, ADORA3, SLA, |
| | RGS13, OLR1, CCL5, CD36, ITGB5, TREM1, ICAM1 |
| GO: 0004872~receptor | PAQR5, CD44, IL10RA, NRP1, CD36, ADORA3, CD180, |
| activity | SLAMF7, NRP2, IL7R, CD36, ADORA3, PAQR5, IFNGR1, |
| | LPAR6, LOC100131909, EMR2, CD244, NRP1, TNFRSF21, |
| | RHBDF1, LILRB1, CSF1R, CXCR7, SUCNR1, TLR4, TH8D, |
| | CD36, P2RY14, CLEC7A, ENPP2, TNFRSF21, THBD, |
| | IFNGR1, TLR4, EPS8, IL7R, CX3CR1, CCR1, PTAFR, CD86, |
| | IL27HA, IL2RG, CXCR7, LILRB2, HMCN1, GPR183, TLR4, |
| | C3AR1, TRPM8, NRP1, GPR35, PTPRO, IFNGR1, TLR4, |
| | ITGB7, ADORA3, THBD, OLR1, CD36, ITGB5, TREM1, |
| | ICAM1 |
| GO: 0044425~membrane | NT5E, PAQR5, FN1, CD44, MFI2, NRP1, CD36, CYP1B1, |
| part | ADORA3, SLAMF7, PCDHGA12, RAB38, IL7R, AGPAT9, |
| | ATP10A, PAQR5, GDPD1, KCNQ3, GLDN, CD109, MPZL3, |
| | IFNGR1, ABHD2, EMR2, GCNT1, CD244, SLC43A2, |
| | FADS3, CYBB, RHBDF1, LILRB1, ABHD2, SLC38A6, |
| | C4ORF34, CSF1R, CXCR7, GPC1, CD36, APBB1IP, CD9, |
| | ATP1B1, GBP2, PUN2, PTGS1, TNFRSF21, THBD, ACPP, |
| | CSPG4, ABHD2, IFNGR1, TLR4, FN1, SLC12A6, TNFSF13B, |
| | CX3CR1, CD52, CCR1, ABMD2, MARCH1, PSD3, LILRB2, |
| | PCDHGA12, SLC7A11, TLR4, GCNT1, AQP1, GBP5, |
| | CALHM2, C3AR1, TRPM8, NRP1, SLC6A6, HAVCR2, |
| | POPDC3, PTPRO, IFNGR1, PTGS1, FADS3, GBP3, |
| | C4ORF34, NRCAM, UGCG, ADAMDEC1, KMO, APBB1IP, |
| | ITGB5, PSD3, ICAM1, GCNT1, ATP1B1, GLIPR1, IL10RA, |
| | SLC37A2, CD180, SLC43A2, NRP2, SYTL1, ST3GAL5, |
| | ATP9A, CYP1B1, DAPP1, DAB2, CD109, GLIPR1, EVL, |
| | CD36, TMEM39A, ADORA3, DSE, ATP6V0D2, PTRF, |
| | PCDH7, NT5E, APBB1IP, STEAP1, TMEM158, SLC41A2, |
| | LPAR6, LOC100131909, HMOX1, CYP1B1, CALHM2, |
| | AQP1, DAB2, GBP2, NRP1, SLAMF8, ABCC3, TM7SF4, |
| | SLC7A11, TNFRSF21, PLA2G4A, LPCAT2, TNFSF13B, |
| | ABHD2, TBXAS1, PTRF, DOCK4, MCOLN3, THBD, TLR4, |
| | SUCNR1, P2RY14, KMO, CLEC7A, ENPP2, LFNG, ABCB1, |
| | EMP1, PTGS1, FN1, GDPD1, FN1, RAB27B, EPS8, DIXDC1, |
| | IL7R, CD52, PARM1, PTAFR, CNIH3, CD86, IL27RA, |
| | CXCR7, IL2RG, SLAMF8, ANKH, RAPH1, MMP14, |
| | GPR183, ABHD2, CKLF, C4ORF34, SLC41A2, MDGA1, |
| | ALCAM, HAVCR2, CYP1B1, RAPH1, TLR4, GPR35, ITGB7, |
| | ADORA3, THBD, SLC41A2, DAB2, OLR1, MCOLN3, DAB2, |
| | CD36, TREM1, ALCAM |
| GO: 0016021~integral to | PAQR5, CD44, MFI2, NRP1, CD36, ADORA3, SLAMF7, |
| membrane | PCDHGA12, IL7R, AGPAT9, ATP10A, PAQR5, GDPD1, |
| | KCNQ3, GLDN, IFNGR1, MPZL3, ABHD2, EMR2, GCNT1, |
| | CD244, SLC43A2, FADS3, CYBB, RHBDF1, LILRB1, ABHD2, |
| | SLC38A6, C4ORF34, CSF1R, CXCR7, GPC1, CD36, CD9, |
| | ATP1B1, TNFRSF21, THBD, ACPP, CSPG4, ABHD2, |
| | IFNGR1, TLR4, SLC12A6, TNFSF13B, CX3CR1, CD52, |
| | CCR1, ABHD2, MARCH1, LILRB2, PCDHGA12, SLC7A11, |
| | TLR4, GCNT1, AQP1, CALHM2, C3AR1, TRPM8, NRP1, |
| | SLC6A6, HAVCR2, POPDC3, IFNGR1, PTPRO, FADS3, |
| | GBP3, C4ORF34, NRCAM, UGCG, ADAMDEC1, KMO, |
| | ITGB5, ICAM1, GCNT1, ATP1B1, GLIPR1, IL10RA, |
| | SLC37A2, CD180, SLC43A2, NRP2, ST3GAL5, ATP9A, |
| | GLIPR1, CD36, TMEM39A, ADORA3, DSE, PCDH7, |
| | STEAP1, TMEM158, LPAR6, SLC41A2, LOC100131909, |
| | AQP1, CALHM2, NRP1, SLAMF8, ABCC3, TM7SF4, |
| | SLC7A11, TNFRSF21, PLA2G4A, LPCAT2, TNFSF13B, |
| | ABHD2, TBXAS1, MCOLN3, THBD, TLR4, SUCNR1, |
| | P2RY14, KMO, CLEC7A, ENPP2, LFNG, ABCB1, EMP1, |
| | GDPD1, EPS8, IL7R, CD52, PARM1, PTAFR, CNIH3, CD86, |
| | IL27RA, CXCR7, IL2RG, SLAMF8, ANKH, MMP14, |
| | GPR183, ABHD2, CKLF, C4ORF34, SLC41A2, HAVCR2, |
| | ALCAM, TLR4, GPR35, ITGB7, ADORA3, THBD, SLC41A2, |
| | OLR1, MCOLN3, CD36, TREM1, ALCAM |
| GO: 0044421~extracellular | THBD, CD44, FN1, FN1, MMP2, VEGFA, ANXA2P1, |
| region part | MMP19, CCL5, ANXA2P1, FN1, TNFSF13B, CD109, IL10, |
| | FGL2, MMP9, HMCN1, VEGFA, MMP14, CCL24, CD109, |
| | VEGFA, MMP1, CKLF, GNLY, HMOX1, COL6A1, A2M, |
| | ADAMTS15, ANXA2P2, CCL5, VEGFA, ANXA2P1, |
| | SERPINE2, TNFSF13B, THBD, IGFBP3, THBD, GPC1, FGL2, |
| | GNLY, COL22A1, CCL5, VASH1, IGFBP3, ICAM1, FN1 |
| GO: 0004888~transmembrane | THBD, IL10RA, NRP1, CD36, ADORA3, IFNGR1, TLR4, |
| receptor activity | NRP2, IL7R, CX3CR1, CCR1, PTAFR, IL7R, CD36, IL27RA, |
| | IL2RG, CXCR7, ADORA3, HMCN1, TLR4, GPR183, |
| | IFNGR1, LPAR6, C3AR1, LOC100131909, EMR2, NRP1, |
| | NRP1, GPR35, TLR4, PTPRO, IFNGR1, ADORA3, THBD, |
| | CSF1R, CXCR7, THBD, TLR4, SUCNR1, OLR1, CD36, |
| | P2RY14, ENPP2, CD36, ICAM1 |
| GO: 0006955~immune | FTHL3, OAS1, VEGFA, OAS1, CCL5, TLR4, CD180, |
| response | SLAMF7, IL7R, TNFSF13B, CCR1, PTAFR, IL7R, CD86, |
| | IL27RA, IL2RG, IL10, LILRB2, VEGFA, TLR4, CCL24, NOD2, |
| | GPR183, VEGFA, GBP5, GBP2, CCL5, VEGFA, TLR4, CYBB, |
| | LILRB1, TNFSF13B, PTX3, GBP3, TLR4, OLR1, P2RY14, |
| | CLEC7A, CCL5, ENPP2, TREM1, GBP2, ICAM1, TRIM22 |
| GO: 0009611~response | FN1, CD44, NRP1, CD36, ADORA3, CD180, CD36 |
| to wounding | ADORA3, ABHD2, HMOX1, NRP1, CYBB, ABHD2, PTX3, |
| | ABHD2, TLR4, THBD, CD36, CLEC7A, CD9, PAPSS2, FN1, |
| | THBD, FN1, ABHD2, CCL5, TLR4, FN1, CX3CR1, CCR1, |
| | PTAFR, ABHD2, IL10, MGLL, HMCN1, CCL24, ABHD2, |
| | TLR4, IRF7, INA, C3AR1, A2M, NRP1, CCL5, NLRC4, TLR4, |
| | THBD, ADORA3, OLR1, CCL5, CD36 |
| GO: 0009605~response | CD44, FN1, NRP1, CD36, ADORA3, CD180, NRP2, CD36, |
| to external stimulus | ADORA3, CDKN1A, NOD2, ABHD2, HMOX1, |
| | LOC100131909, NRP1, CYBB, PLA2G4A, PTX3, ABHD2, |
| | ABHD2, TLR4, THBD, CD36, CLEC7A, ENPP2, PAPSS2, |
| | CD9, FN1, THBD, FN1, ABHD2, CCL5, TLR4, FN1, CX3CR1, |
| | CCR1, PTAFR, ABHD2, IL10, MGLL, HMCN1, MMP14, |
| | IRF7, CCL24, ABHD2, TLR4, CKLF, INA, C3AR1, A2M, |
| | NRP1, CCL5, NLRC4, TLR4, ADORA3, THBD, CCND1, |
| | OLR1, CCL5, CD36 |
| GO: 0002376~immune | OAS1, OAS1, CD180, SLAMF7, IL7R, MMP9, NOD2, |
| system process | EPAS1, GBP2, ID2, TM7SF4, CYBB, LILRB1, TNFSF13B, |
| | BCL6, PTX3, IFI30, TLR4, P2RY14, CLEC7A, ENPP2, GBP2, |
| | TRIM22, FTHL3, VEGFA, CCL5, ID2, TLR4, IL7R, |
| | TNFSF13B, CCR1, PTAFR, CD86, IL27RA, IL2RG, IL10, |
| | LILRB2, VEGFA, CCL24, GPR183, TLR4, VEGFA, GBP5, |
| | CKLF, C3AR1, BCL6, CCL5, VEGFA, TLR4, CRIP2, ID2, |
| | GBP3, OLR1, CCL5, TREM1, ICAM1 |
| GO: 0050896~response | FN1, CD44, OAS1, NRP1, CD36, CYP1B1, ADORA3, |
| to stimulus | SLAMF7, IL7R, EEPD1, CDKN1A, IFNGR1, ABHD2, ID2, |
| | CYBB, LILRB1, BCL6, ABHD2, GNLY, CD36, CD9, PAPSS2, |
| | ATP1B1, GBP2, PLIN2, TRIM22, PTGS1, FTHL3, THBD, |
| | VEGFA, ABHD2, IFNGR1, CCL5, TLR4, ID2, FN1, |
| | TNFSF138, CX3CR1, ME1, CCR1, ABHD2, IL10, LILRB2, |
| | HMCN1, SLC7A11, AQP1, CCL24, TLR4, IRF7, GBP5, |
| | C3AR1, RASGRF1, TRPM8, A2M, ME1, NRP1, VEGFA, |
| | IFNGR1, PTGS1, GBP3, EEPD1, HIPK2, KMO, ICAM1, |
| | ATP1B1, MMP2, OAS1, FGD4, CD180, NRP2, CYP1B1, |
| | CD36, ADORA3, NOD2, EPAS1, GNLY, LOC100131909, |
| | HMOX1, CYP1B1, AQP1, IFI44, GBP2, NRP1, SLC7A11, |
| | PLA2G4A, PTX3, TNFSF13B, ABHD2, MCOLN3, TLR4, |
| | THBD, P2RY14, KMO, CLEC7A, ENPP2, ABCB1, PTGS1, |
| | FN1, FN1, EPS8, IL7R, HSPB7, PTAFR, CD86, IL27RA, |
| | IL2RG, MGLL, VEGFA, ANKH, MMP14, ABHD2, GPR183, |
| | VEGFA, CKLF, INA, BCL6, FABP4, CCL5, NLRC4, CYP1B1, |
| | FGD4, TLR4, ID2, THBD, ADORA3, CCND1, OLR1, |
| | MCOLN3, CCL5, CD36, TREM1 |
| GO: 00322501~multicellular | ADAP2, PAQR5, CD44, NRP1, CD36, CYP1B1, LBH, IL7R, |
| organismal process | KCNQ3, MMP9, PAQR5, MAF, GLDN, CDKN1A, MPZL3, |
| | PDLIM7, ID2, ANXA2P1, TFAP2A, ATF5, MITF, BCL6, |
| | CSF1R, CKB, CD36, NGEF, CD9, PAPSS2, IGFBP3, PTGS1, |
| | THBD, CSPG4, VEGFA, TLR4, ID2, PRDM1, SLC12A6, |
| | TRPS1, STATH, IL10, HMCN1, MAFB, AQP1, TLR4, |
| | C3AR1, RASGRF1, TRPM8, NRP1, VEGFA, SKIL, PTGS1, |
| | NRCAM, ARI05B, UGCG, HIPK2, MMP2, TRPS1, NRP2, |
| | CYP1B1, DAB2, CD36, EVL, ZFP36L1, NOD2, EPAS1, |
| | TUBB2A, LOC100131909, HMOX1, CYP1B1, AQP1, |
| | DAB2, ATF5, ANXA2P2, NRP1, TM7SF4, CDK5RAP2, |
| | PLA2G4A, SERPINE2, IGFBP3, LMNA, MCOLN3, TLR4, |
| | THBD, CLEC7A, LFNG, TRPS1, AHNAK, PGA5, EMP1, |
| | PTGS1, ANXA2P1, MMP19, NGEF, ANXA2P1, EPS8, |
| | DIXDC1, IL7R, TPRS1, MAFB, PTAFR, MITF, NPTX1, |
| | VEGFA, ANKH, MMP14, GPR183, MMP1, VEGFA, INA, |
| | BCL6, MDGA1, PDLIM7, ALCAM, FABP4, CDK5RAP2, |
| | NLRC4, CYP1B1, TLR4, CRIP2, ID2, THBD, CCND1, DAB2, |
| | ADAP2, OLR1, MCOLN3, DAB2, CD36, ALCAM |
| GO: 0050793~regulation | FN1, NRP1, CD36, FGD4, IL7R, CD36, ATP10A, MAF, |
| of developmental | PDLIM7, HMOX1, LOC100131909, ATF5, ID2, NRP1, |
| process | CDK5RAP2, ATF5, MITF, PLA2G4A, BCL6, IGFBP3, FGD2, |
| | TLR4, CD36, IGFBP3, FN1, FN1, VEGFA, FGD2, CCL5, ID2, |
| | TLR4, FN1, IL7R, MAFB, MITF, CD86, STATH, IL27RA, |
| | IL2RG, IL10, VEGFA, ANKH, MAFB, TLR4, VEGFA, BCL6, |
| | PDLIM7, CDK5RAP2, NRP1, CCL5, VEGFA, FGD4, FGD2, |
| | TLR4, ID2, FGD2, CCND1, NRCAM, CCL5, VASH1, CD36 |
| GO: 0009897~external | CD44, TRPM8, CSPG4, ALCAM, CD244, TLR4, TLR4, IL7R, |
| side of plasma | IL7R, NRCAM, CD86, IL2RG, TLR4, GPC1, CLEC7A, TLR4, |
| membrane | ALCAM, ICAM1 |
| GO: 0051239~regulation | NRP1, ADORA3, IL7R, ADORA3, MAF, NOD2, EPAS1, |
| of multicellular | PDLIM7, HMOX1, LOC100131909, ATF5, ANXA2P2, ID2, |
| organismal process | NRP1, ANXA2P1, CDK5RAP2, ATF5, MITF, PLA2G4A, |
| | BCL6, IGFBP3, TLR4, CLEC7A, PTGS1, IGFBP3, PTGS1, |
| | VEGFA, ANXA2P1, CCL5, ID2, ANXA2P1, TLR4, IL7R, |
| | HSPB7, MAFB, MITF, CD86, STATH, IL27RA, IL2RG, IL10, |
| | VEGFA, ANKH, MAFB, TLR4, VEGFA, RASGRF1, BCL6, |
| | PDLIM7, CDK5RAP2, NRP1, CCL5, VEGFA, TLR4, ID2, |
| | PTGS1, ADORA3, NRCAM, CCND1, CCL5, VASH1 |
| GO: 0006952~defense | CD44, FN1, FN1, ADORA3, CCL5, TLR4, CD180, FN1, |
| response | SLAMF7, CX3CR1, CCR1, PTAFR, IL27RA, ADORA3, IL10, |
| | MGLL, LILRB2, IRF7, TLR4, CCL24, NOD2, GNLY, C3AR1, |
| | HMOX1, LOC100131909, A2M, CCL5, NLRC4, TLR4, |
| | CYBB, PTX3, ADORA3, OLR1, TLR4, GNLY, CLEC7A, CCL5, |
| | FN1 |
| GO: 0006954~inflammatory | FN1, FN1, CD44, ADORA3, CCL5, CD180, TLR4, FN1, |
| response | CCR1, PTAFR, MGLL, ADORA3, IL10, TLR4, CCL24, IRF7, |
| | C3AR1, HMOX1, A2M, CCL5, NLRC4, TLR4, CYBB, PTX3, |
| | ADORA3, TLR4, OLR1, CLEC7A, CCL5, FN1 |
| GO: 0031012~extracellular | CD44, FN1, FN1, MMP2, COLGA1, VEGFA, ANXA2P1, |
| matrix | MMP19, ANXA2P2, ADAMTS15, VEGFA, ANXA2P1, |
| | ANXA2P1, FN1, GPC1, COL22A1, MMP9, HMCN1, |
| | VEGFA, MMP14, VEGFA, MMP1, FN1 |
| GO: 0048583~regulation | NT5E, VEGFA, ANXA2P1, FGD4, CCL5, ANXA2P1, TLR4, |
| of response to stimulus | IL7R, TMFSF13B, IL7B, CD86, IL27RA, IL10, VEGFA, |
| | NOD2, TLR4, IRF7, VEGFA, NT5E, C3AR1, HMOX1, |
| | LOC100131909, BCL6, A2M, FABP4, ANXA2P2, CCL5, |
| | VEGFA, FGD4, AMXA2P1, TLR4, PLA2G4A, TNFSF138, |
| | BCL6, TLR4, HIPK2, CLEC7A, CCL5, ICAM1 |
| GO: 0005578~proteinaceous | FN1, FN1, MMP2, COL6A1, VEGFA, ANXA2P1, MMP19, |
| extracellular matrix | ANXA2P2, ADAMTS15, VEGFA, ANXA2P1, ANXA2P1, |
| | FN1, GPC1, COL22A1, MMP9, HMCN1, VEGFA, MMP14, |
| | VEGFA, MMP1, FN1 |
| GO: 0009986~cell surface | CD44, CSPG4, VEGFA, CD36, TLR4, IL7R, IL7R, CD36, |
| | CD86, IL2RG, VEGFA, NOD2, TLR4, VEGFA, TRPM8, |
| | ALCAM, CD244, VEGFA, TM7SF4, TLR4, NRCAM, TLR4, |
| | GPC1, CD36, CLEC7A, CD36, ABCB1, ALCAM, ICAM1 |
| GO: 0002682~regulation | CD44, VEGFA, CCL5, ID2, TLR4, IL7R, TNFSF13B, MAFB, |
| of immune system | IL7R, MITF, CD86, IL27RA, IL2RG, IL10, VEGFA, CDKN1A, |
| process | MAFB, NOD2, TLR4, VEGFA, C3AR1, HMOX1, |
| | LOC100131909, BCL6, A2M, CCL5, VEGFA, ID2, TLR4, |
| | MITF, TNFSF13B, ID2, BCL6, TLR4, CLEC7A, CCL5, ICAM1 |
| GO: 0005615~extracellular | THBD, FN1, FN1, MMP2, VEGFA, CCL5, FN1, TNFSF13B, |
| space | CD109, IL10, FGL2, MMP9, VEGFA, CD109, CCL24, |
| | VEGFA, CKLF, GNLY, HMOX1, A2M, CCL5, VEGFA, |
| | SERPINE2, TNFSF13B, IGFBP3, THBD, THBD, GPC1, |
| | GNLY, FGL2, CCL5, VASH1, IGFBP3, ICAM1, FN1 |
| GO: 0048731~system | ADAP2, CD44, MMP2, NRP1, TRPS1, NRP2, IL7R, EVL, |
| development | ZFP36L1, MAF, MMP9, CDKN1A, GLDN, EPAS1, TUBB2A, |
| | PDLIM7, HMOX1, ATF5, ANXA2P2, ID2, NRP1, ANXA2P1, |
| | TM7SF4, CDXSRAP2, ATF5, TFAP2A, PLA2G4A, MITF, |
| | SERPINE2, BCL6, IGFBP3, LMNA, MCOLN3, CKB, LFNG, |
| | NGEF, PAPSS2, CD9, TRPS1, AHNAK, IGFBP3, EMP1, |
| | ANXA2P1, CSPG4, VEGFA, MMP19, NGEF, ID2, |
| | ANXA2P1, PRDM1, IL7R, TRPS1, MAFB, MITF, STATH, |
| | TRPS1, NPTX1, IL10, MMP14, ANKH, MAFB, VEGFA, |
| | GPR183, VEGFA, INA, RASGRF1, BCL6, MDGA1, PDLIM7, |
| | ALCAM, CDK5RAP2, NRP1, VEGFA, CRIP2, ID2, NRCAM, |
| | CCND1, ARIDSB, UGCG, ADAP2, MCOLN3, ALCAM |
| GO: 0051707~response | THBD, CCL5, IFNGR1, TLR4, PTAFR, IL27RA, IL10, TLR4, |
| to other organism | NOD2, IRF7, IFNGR1, GNLY, IFI44, CCL5, NLRC4, TLR4, |
| | IFNGR1, LILRB1, PLA2G4A, PTX3, THBD, TLR4, THBD, |
| | GNLY, CLEC7A, CCL5, TRIM22 |
| GO: 0007275~multicellular | PAQR5, ADAP2, CD44, MMP2, NRP1, TRPS1, LBH, NRP2, |
| organismal | DAB2, IL7R, EVL, ZFP36L1, MAF, PAQR5, MMP9, |
| development | CDKN1A, GLDN, EPAS1, TUBB2A, PDLIM7, HMOX1, |
| | DAB2, ATF5, ANXA2P2, ID2, NRP1, ANXA2P1, TM7SF4, |
| | CDK5RAP2, ATF5, TFAP2A, PLA2G4A, MITF, SERPINE2, |
| | BCL6, IGFBP3, CSF1R, LMNA, MCOLN3, CKB, THBD, |
| | LFNG, NGEF, PAPSS2, CD9, TRPS1, AHNAK, IGFBP3, |
| | EMP1, THBD, VEGFA, ANXA2P1, CSPG4, MMP19, NGEF, |
| | ID2, ANXA2P1, PRDM1, DIXDC1, IL7R, TRPS1, MAFB, |
| | MITF, STATH, TRPS1, NPTX1, IL10, MMP14, ANKH, |
| | VEGFA, MAFB, GPR183, VEGFA, INA, RASGRF1, BCL6, |
| | MDGA1, PDLIM7, ALCAM, CDK5RAP2, NRP1, VEGFA, |
| | CRIP2, SKIL, ID2, THBD, NRCAM, CCND1, ARID5B, UGCG, |
| | DAB2, ADAP2, HIPK2, MCOLN3, DAB2, ALCAM |
| GO: 0032101~regulation | NT5E, LOC100131909, BCL6, VEGFA, ANXA2P1, A2M, |
| of response to external | FABP4, ANXA2P2, CCL5, CCL5, VEGFA, TLR4, ANXA2P1, |
| stimulus | AMXA2P1, TLR4, PLA2G4A, BCL6, TLR4, IL10, VEGFA, |
| | CCL5, TLR4, VEGFA, NT5E |
| GO: 0022610~biological | CD44, FN1, FN1, NRP1, CD36, CCL5, FN1, SLAMF7, |
| adhesion | MTSS1, NRP2, PCDHGA12, CX3CR1, CCR1, CD36, |
| | PCDHGA12, RAPH1, PCDH7, MPZL3, COL6A1, ALCAM, |
| | CCL5, NRP1, RAPH1, NRP1, MTSS1, ITGB7, LPXN, |
| | NRCAM, OLR1, CD36, CLEC7A, COL22A1, CCL5, CD36, |
| | ITGB5, CD9, ALCAM, ICAM1, FN1 |
| GO: 0007155~cell | CD44, FN1, FN1, NRP1, CD36, CCL5, FN1, SLAMF7, |
| adhesion | MTSS1, NRP2, PCDHGA12, CX3CR1, CCR1, CD36, |
| | PCDHGA12, RAPH1, PCDH7, MPZL3, COL6A1, ALCAM, |
| | CCL5, NRP1, RAPH1, NRP1, MTSS1, ITG87, LPXN, |
| | NRCAM, OLR1, CD36, CLEC7A, COL22A1, CCL5, CD36, |
| | ITGB5, CD9, ALCAM, ICAM1, FN1 |
| GO: 0048856~anatomical | ADAP2, FN1, CD44, MMP2, NRP1, TRPS1, NRP2, DAB2, |
| structure development | IL7R, EVL, ZFP36L1, MAF, MMP9, CDKN1A, GLDN, |
| | EPAS1, TUB82A, PDLIM7, HMOX1, DAB2, ATF5, |
| | ANXA2P2, ID2, NRP1, ANXA2P1, TM7SF4, CDK5RAP2, |
| | ATF5, TFAP2A, PLA2G4A, MITF, SERPINE2, BCL6, IGFBP3, |
| | LMNA, MCOLN3, CKB, LFNG, NGEF, PAPSS2, CD9, TRPS1, |
| | AHNAX, IGFBP3, EMP1, FN1, FN1, ANXA2P1, CSPG4, |
| | VEGFA, MMP19, HGEF, ID2, ANXA2P1, PRDM1, FN1, |
| | IL7B, TRPS1, MAFB, MITF, STATH, TRPS1, NPTX1, IL10, |
| | MMP14, ANKH, MAFB, VEGFA, GPR183, VEGFA, INA, |
| | RASGRF1, BCL6, MDGA1, PDLIM7, ALCAM, CDK5RAP2, |
| | NRP1, VEGFA, CRIP2, ID2, NRCAM, CCND1, ARID5B, |
| | UGCG, DAB2, ADAP2, MCOLN3, DAB2, ALCAM |
| GO: 0065008~regulation | MFI2, FN1, NRP1, CD36, CYP1B1, FGD4, IL7R, DAB2, |
| of biological quality | CYP1B1, CD36, ATP10A, CDKN1A, EPAS1, HMOX1, |
| | LOC100131909, CYP181, DAB2, ANXA2P2, ID2, NRP1, |
| | ANXA2P1, PLA2G4A, TNFSF13B, BCL6, CKB, FGD2, THBD, |
| | TLR4, IFI30, CD36, PAPSS2, CD9, FTGS1, EMP1, PTGS1, |
| | FN1, FTHL3, THBD, FN1, VEGFA, ANXA2P1, FGD2, CCL5, |
| | TLR4, ID2, ANXA2P1, FN1, SLC12A6, IL7R, TNFSF13B, |
| | CD52, CD52, CCR1, STATH, IL10, HMCN1, VEGFA, IRF7, |
| | TLR4, VEGFA, C3AR1, RASGRF1, BCL6, TRPM8, FABP4, |
| | CCL5, NRP1, VEGFA, CYP1B1, FGD4, FGD2, TLR4, ID2, |
| | THBD, PTGS1, FGD2, DAB2, CCL5, CD36, DAB2, SERTAD1 |
| GO: 0007166~cell surface | NRP1, ADORA3, MTSS1, MAML3, IL7R, EVL, ADORA3, |
| receptor linked signal | MAML2, LPAR6, LOC100131909, EMR2, NRP1, MAML2, |
| transduction | MITF, CSF1R, CXCR7, SUCNR1, P2RY14, CLEC7A, ENPP2, |
| | VEGFA, CCL5, MAML2, EPS8, DIXDC1, IL7R, CK3CR1, |
| | CCR1, PTAFR, MITF, IL27RA, CXCR7, LILRB2, VEGFA, |
| | GPR183, VEGFA, MAML2, C3AR1, MAML2, NRP1, CCL5, |
| | VEGFA, DOK2, MTSS1, GPR35, ITGB7, ADORA3, CCND1, |
| | ARID5B, RGS13, ADAMDEC1, HIPK2, CCL5, ITGB5 |
| GO: 0050727~regulation | NT5E, BCL6, LOC100131909, A2M, FABP4, CCL5, CCL5, |
| of inflammatory | TLR4, TLR4, PLA2G4A, BCL6, TLR4, IL10, CCL5, TLR4, |
| response | NT5E |
| GO: 0006950~response | FN1, CD44, MMP2, NRP1, CD36, FGD4, ADORA3, CD180, |
| to stress | SLAMF7, CD36, ADORA3, CDKN1A, EEPD1, NOD2, |
| | EPA51, GNLY, ABHD2, HMOX1, LOC100131909, NRP1, |
| | CYBB, PLA2G4A, BCL6, ABHD2, PTX3, ABHD2, TLR4, |
| | THBD, GNLY, CD36, KMO, CLEC7A, CD9, PAPSS2, |
| | ATP1B1, PTGS1, FN1, PTGS1, THBD, FN1, VEGFA, |
| | ABHD2, CCL5, TLR4, FN1, HSPB7, CX3CR1, CCR1, PTAFR, |
| | ABHD2, IL27RA, MGU, IL10, LILRB2, HMCN1, MMP14, |
| | VEGFA, CCL24, ABHD2, TLR4, IRF7, VEGFA, INA, C3AR1, |
| | BCL6, TRPM8, A2M, NRP1, CCL5, NLRC4, FGD4, VEGFA, |
| | TLR4, PTGS1, THBD, ADORA3, CCND1, OLR1, EEPD1, |
| | HIPK2, KMO, CCL5, CD36, ATP1B1 |
| GO: 0032502~developmental | ADAP2, PAQR5, CD44, FN1, NRP1, LBH, IL7R, MMP9, |
| process | PAQRS, MAF, GLDN, CDKN1A, PDLIM7, ID2, ANXA2P1, |
| | TFAP2A, ATF5, MITF, BCL6, CSF1R, CKB, NGEF, CD9, |
| | PAPSS2, IGFBP3, PTGS1, THBD, CSPG4, VEGFA, CCL5, |
| | ID2, FN1, PRDM1, TRPS1, STATH, IL10, MAFB, RASGRF1, |
| | NRP1, VEGFA, SKIL, PTGS1, ARID5B, NRCAM, UGCG, |
| | HIPK2, MMP2, TRPS1, NRP2, DAB2, EVL, ZFP36L1, |
| | EPAS1, TUBB2A, HMOX1, DAB2, ATFS, ANXA2P2, NRP1, |
| | TM7SF4, CDK5RAP2, SERPINE2, PLA2G4A, IGFBP3, |
| | LMNA, MCOLN3, THBD, LFNG, TRPS1, AHNAK, EMP1, |
| | PTGS1, FN1, FN1, ANXA2P1, MMP19, NGEF, ANXA2P1, |
| | DIXDC1, IL7R, TRPS1, MAFB, MITF, NPTX1, VEGFA, |
| | ANKH, MMP14, GPR183, VEGFA, INA, BCL6, MDGA1, |
| | PDLIM7, ALCAM, FABP4, CDK5RAP2, CCL5, CRIP2, ID2, |
| | THBD, CCND1, ADAP2, DAB2, MCOLN3, CCL5, DAB2, |
| | ALCAM |
| GO: 0031347~regulation | NT5E, LOC10013I909, BCL6, A2M, FABP4, CCL5, CCL5, |
| of defense response | TLR4, TLR4, PLA2G4A, BCL6, TLR4, IL10, CLEC7A, CCL5, |
| | TLR4, IRF7, NOD2, NT5E |
| GO: 0009607~response | THBD, CCL5, IFNGR1, TLR4, HSPB7, PTAFR, IL27RA, IL10, |
| to biotic stimulus | TLR4, NOD2, IRF7, IFNGR1, GNLY, IFI44, CCL5, NLRC4, |
| | IFNGR1, TLR4, LILRB1, PLA2G4A, PTX3, THBD, CCND1, |
| | TLR4, THBD, GNLY, CLEC7A, CCL5, TRIM22 |
| GO: 0019955~cytokine | IL10RA, CD36, A2M, IFNGR1, IL7R, IFNGR1, CX3CR1, |
| binding | CCR1, IL7R, CSF1R, CD36, IL27RA, IL2RG, CD36, CD36, |
| | IFNGR1 |
| GO: 0050776~regulation | C3AR1, HMOX1, LOC100131909, BCL6, A2M, TLR4, |
| of immune response | TLR4, IL7R, TNFSF138, TNFSF138, BCL6, IL7R, CD86, |
| | IL27RA, TLR4, IL10, CLEC7A, NOD2, TLR4, ICAM1 |
| GO: 0040011~locomotion | FN1, FN1, CD44, NRP1, CCLS, FN1, NRP2, CX3CR1, |
| | S100A2, CCR1, PTAFR, IL10, MMP14, CCL24, CKLF, |
| | C3AR1, MDGA1, CCL5, NRP1, NRP1, ARID58, NRCAM, |
| | CCL5, ENPP2, ICAM1, FN1 |
| GO: 0080134~regulation | NTSE, ANXA2P1, FGD4, CCLS, ANXA2P1, TLR4, IL10, |
| of response to stress | NOD2, TLR4, IRF7, NT5E, BCL6, LOC100131909, A2M, |
| | FABP4, ANXA2P2, CCL5, FGD4, ANXA2P1, TLR4, |
| | PLA2G4A, BCL6, TLR4, HIPK2, CLEC7A, CCL5 |
| GO: 0007626~locomotory | C3AR1, CCL5, CCL5, EPS8, NRP2, CX3CR1, CCR1, PTAFR, |
| behavior | MCOLN3, HIPK2, IL10, MCOLN3, ANKH, CCL5, ENPP2, |
| | CCL24, CKLF |
| GO: 0002684~positive | C3AR1, LOC100131909, BCL6, VEGFA, VEGFA, TLR4, |
| regulation of immune | IL7R, TLR4, TNFSF13B, TNFSF13B, BCL6, IL7R, CD86, |
| system process | IL27RA, IL2RG, TLR4, CLEC7A, VEGFA, CDKN1A, NOD2, |
| | TLR4, VEGFA, ICAM1 |
| GO: 0000267~cell | NT5E, OAS1, CD36, CYP1B1, OAS1, SYTL1, PCDHGA12, |
| fraction | NRP2, RAB38, CYP1B1, CD36, PTRF, NTSE, HMOX1, |
| | CYP1B1, FADS3, ANXA2P1, ABCC3, PLA2G4A, TNFSF13B, |
| | TBXAS1, PTRF, LMNA, CD36, ABCB1, EMP1, PTGS1, |
| | PTGS1, ANXA2P1, CCL5, ANXA2P1, EPS8, SLC12A6, |
| | TNFSF13B, ME1, CD52, CD52, PSD3, LILRB2, PCDHGA12, |
| | RASGRF1, ME1, FABP4, CCL5, CYP1B1, PTGS1, FADS3, |
| | UGCG, OLR1, CTSL1, CCL5, CD36, PSD3 |
| GO: 0045028~purinergic | LPAR6, LOC100131909, SUCNR1, P2RY14, ADORA3, |
| nudeotide receptor | ADORA3, GPR183, ADORA3 |
| activity, G-protein | |
| coupled | |
| GO: 0001608~nucleotide | LPAR6, LOC100131909, SUCNR1, P2RY14, ADORA3, |
| receptor activity, G- | ADORA3, GPR183, ADORA3 |
| protein coupled | |
| GO: 0004930~G-protein | LPAR6, C3AR1, LOC100131909, EMR2, ADORA3, GPR35, |
| coupled receptor activity | CX3CR1, CCR1, PTAFR, ADORA3, CXCR7, CXCR7, |
| | SUCNR1, P2RY14, ADORA3, GPR183 |
| GO: 0016502~nucleotide | LPAR6, LOC100131909, SUCNR1, P2RY14, ADORA3, |
| receptor activity | ADORA3, GPR183, ADORA3 |
| GO: 0001614~purinergic | LPAR6, LOC100131909, SUCNR1, P2RY14, ADORA3, |
| nucleotide receptor | ADORA3, GPR183, ADORA3 |
| GO: 0005626~insoluble | NT5E, OAS1, CD36, OAS1, CYP1B1, EPS8, SLC12A6, |
| friction | NRP2, SYTL1, PCDHGA12, CD52, CD52, RAB38, CYP1B1, |
| | PSD3, CD36, ULRB2, PCDHGA12, PTRF, NT5E, RASGRF1, |
| | HMOX1, CYP1B1, CYP1B1, FADS3, ABCC3, PLA2G4A, |
| | PTRF, TBXAS1, PTGS1, FADS3, LMNA, UGCG, OLR1, |
| | CD36, CD36, PSD3, ABCB1, PTGS1, EMP1, PTGS1 |
| GO: 0048513~organ | ADAP2, CD44, MMP2, NRP1, NRP2, IL7R, EVL, ZFP36L1, |
| development | MMP9, MAF, CDKN1A, EPAS1, HMOX1, ANXA2P2, |
| | NRP1, ID2, ANXA2P1, TM7SF4, CDK5RAP2, TFAP2A, |
| | MITF, PLA2G4A, BCL6, LMNA, MCOLN3, CKB, LFNG, |
| | EMP1, ANXA2P1, CSPG4, VEGFA, MMP19, ANXA2P1, |
| | ID2, PRDM1, IL7R, MAFB, MITF, STATH, IL10, VEGFA, |
| | MMP14, MAFB, GPR183, VEGFA, INA, BCL6, MDGA1, |
| | CDKSRAP2, NRP1, VEGFA, CRIP2, ID2, CCND1, ARID5B, |
| | UGCG, ADAP2, MCOLN3 |
| GO: 0002697~regulation | IL7R, HMOX1, LOC100131909, BCL6, IL27RA, A2M, IL10, |
| of immune effector | NOD2, IL7R, ICAM1, BCL6 |
| process | |
| GO: 0051384~response | CCND1, A2M, FABP4, IL10, CCL5, CCL5, CDKN1A, CCL5, |
| to glucocorticoid | PTGS1, PLA2G4A, PTGS1, PTGS1 |
| stimulus | |
| GO: 0005624~membrane | NT5E, OAS1, CD36, OAS1, CYP1B1, EPS8, SLC12A6, |
| fraction | NRP2, SYTL1, PCDHGA12, CD52, CD52, RAB38, CYP1B1, |
| | PSD3, CD36, LILRB2, PCDHGA12, PTRF, NT5E, RASGRF1, |
| | HMOX1, CYP1B1, CYP1B1, FADS3, ABCC3, PLA2G4A, |
| | PTRF, TBXAS1, PTGS1, FADS3, UGCG, OLR1, CD36, CD36, |
| | PSD3, ABCB1, PTG51, EMP1, PTGS1 |
| hsa04060:Cytokine- | TNFRSF21, IL10RA, VEGFA, IFNGR1, CCL5, CCL5, VEGFA, |
| cytokine receptor | IFNGR1, IL7R, TNFSF13B, TNFRSF21, CX3CR1, TNFSF13B, |
| interaction | CCR1, CSF1R, IL7R, IL2RG, IL10, VEGFA, CCL5, CCL24, |
| | VEGFA, IFNGR1 |
| GO: 0031349~positive | LOC100131909, FABP4, CCL5, CCL5, TLR4, TLR4, |
| regulation of defense | PLA2G4A, TLR4, CLEC7A, CCL5, TLR4, IRF7, NOD2 |
| response | |
| GO: 0048584~positive | C3AR1, LOC100131909, VEGFA, FABP4, CCL5, CCL5, |
| regulation of response to | VEGFA, TLR4, TLR4, TNFSF13B, PLA2G4A, TNFSF13B, |
| stimulus | IL27RA, TLR4, HIPK2, CLEC7A, VEGFA, CCL5, NOD2, |
| | TLR4, IRF7, VEGFA |
| GO: 0031960~response | CCND1, A2M, FABP4, IL10, CCL5, CCL5, CDKN1A, CCL5, |
| to corticosteroid | PTGS1, PLA2G4A, PTGS1, PTGS1 |
| stimulus | |
| GO: 0042221~response | CD44, MMP2, CYP1B1, NRP2, CYP1B1, CDKN1A, NOD2, |
| to chemical stimulus | EPAS1, HMOX1, AQP1, CYP1B1, ID2, SLC7A11, PLA2G4A, |
| | TLR4, THBD, CLEC7A, ENPP2, ATP1B1, ABCB1, PTGS1, |
| | PLIN2, PTGS1, THBD, VEGFA, CCL5, ID2, TLR4, EPS8, |
| | HSPB7, ME1, CX3CR1, CCR1, PTAFR, IL10, SLC7A11, |
| | VEGFA, MMP14, AQP1, CCL24, TLR4, VEGFA, CKLF, |
| | C3AR1, A2M, ME1, FABP4, CCL5, VEGFA, CYP1B1, TLR4, |
| | ID2, PTGS1, THBD, CCND1, OLR1, CCL5, ATP1B1 |
| GO: 0065007~biological | TCF4, NT5E, MFI2, NRP1, ADORA3, SLAMF7, RAB38, |
| regulation | IL7R, PDK4, MAF, PHLDA1, MAML2, ARHGAP18, CD244, |
| | ID2, ANXA2P1, RAB7B, RIN2, ATF5, TFAP2A, MITF, BCL6, |
| | ABHD2, CSF1R, CKB, FGL2, TCF4, CD36, IGFBP3, PTGS1, |
| | FTHL3, HIVEP1, TNFRSF21, ABHD2, CCL5, ID2, PRDM1, |
| | FN1, TNFSF13B, TCF4, CX3CR1, CCR1, TRPS1, STATH, |
| | IL10, HMCN1, MAFB, TCF4, RASGRF1, A2M, FGD2, |
| | CYTIP, IFNGR1, SKIL, PHLDA1, LPXN, PTSS1, FGD2, |
| | NRCAM, ADAMDEC1, ICAM1, FGD4, MAML3, CYP1B1, |
| | DAPP1, CD36, ZFP36L1, ARHGAP18, PTRF, NOD2, EPAS1, |
| | ARHGAP18, HMOX1, LOC100131909, ANXA2P2, NRP1, |
| | CDK5RAP2, TNFRSF21, PLA2G4A, SERPINE2, TNFSF13B, |
| | IGFBP3, PTRF, FGD2, SUCNR1, THBO, P2RY14, CLEC7A, |
| | TRPS1, PTGS1, ARHGEF3, NGEF, ANXA2P1, IL7R, HSPB7, |
| | TRPS1, CD52, MITF, CD86, IL27RA, CKCR7, FGL2, VEGFA, |
| | RAPH1, VEGFA, MAML2, BCL6, MAML2, PDLIM7, |
| | ALCAM, FABP4, RAPH1, FGD4, GPR35, ID2, RGS13, |
| | ADAP2, DAB2, DAB2, ALCAM, ADAP2, FN1, CD44, CD36, |
| | CYP1B1, LBH, MTSS1, PHLDA1, AGPAT9, MMP9, DDIT4L, |
| | ATP10A, CDKN1A, IFNGR1, PDLIM7, ABHD2, EMR2, |
| | RGL1, TCF4, RAB7B, CXCR7, IFI30, APBB1IP, NGEF, CD9, |
| | PAPSS2, TRIM22, THBD, VEGFA, CSPG4, IFNGR1, |
| | MAML2, TLR4, SLC12A6, CDS2, C9ORF89, ABHD2, PSD3, |
| | CCL24, TLR4, IRF7, C3AR1, TRPM8, NRP1, VEGFA, |
| | ARID5B, TCF4, HIPK2, APBB1IP, VASH1, PSD3, TRPS1, |
| | DAB2, ADORA3, SNAI3, APBB1IP, NT5E, LPAR6, CYP1B1, |
| | DAB2, ATF5, MAML2, PTX3, ABHD2, TLR4, ENPP2, GLIS3, |
| | EMP1, BCL2A1, FN1, FN1, ANXA2P1, FGD2, RAB27B, |
| | EPS8, MAFB, PTAFR, CNIH3, IL2RG, PDK4, MMP14, |
| | ANKH, GPR183, ABHD2, ARHGAP20, CDX5RAP2, CCL5, |
| | NLRC4, CYP1B1, MTSS1, DOK2, TLR4, CRIP2, THBD, |
| | ADORA3, CCND1, CCL5, CD36, CHD9, TREM1, SERTAD1 |
| GO: 0050865~regulation | HMOX1, BCL6, LOC100131909, TLR4, TLR4, IL7R, |
| of cell activation | TNFSF13B, TNFSF13B, BCL6, IL7R, CD86, IL27RA, TLR4, |
| | IL2RG, IL10, CDKN1A, TLR4 |
| GO: 0048545~response | HMOX1, AQP1, FABP4, A2M, CCL5, CCL5, PIA2G4A, |
| to steroid hormone | PTGS1, CCND1, IL10, CCL5, MMP14, CDKN1A, AQP1, |
| stimulus | PTGS1, PTGS1 |
| GO: 0006935~chemotaxis | C3AR1, IL10, CCL5, CCL5, CCL5, ENPP2, CCL24, NRP2, |
| | CKLF, CX3CR1, CCR1, PTAFR |
| GO: 0042330~taxis | C3AR1, IL10, CCL5, CCL5, CCL5, ENPP2, CCL24, NRP2, |
| | CKLF, CX3CR1, CCR1, PTAFR |
| GO: 0002703~regulation | IL7R, HMOX1, LOC100131909, BCL5, IL27RA, IL10, |
| of leukocyte mediated | NOD2, IL7R, BCL6 |
| immunity | |
| GO: 0002822~regulation | IL7R, BCL6, CD86, IL27RA, IL10, NOD2, IL7R, TNFSF13B, |
| of adaptive immune | TNFSF13B, BCL6 |
| response based on | |
| somatic recombination | |
| of immune receptors | |
| built from | |
| immunoglobulin | |
| superfamily domains | |
| G0: 0001568~blood | CD44, HMOX1, MMP2, CSPG4, NRP1, ANXA2P1, VEGFA, |
| vessel development | MMP19, ANXA2P2, NRP1, NRP1, ANXA2P1, VEGFA, |
| | ANXA2P1, NRP2, ZFP36L1, MMP14, VEGFA, VEGFA, |
| | EPAS1 |
| G0: 0030154~cell | PAQR5, FN1, CD44, NRP1, NRP2, IL7R, DAB2, ZFP36L1, |
| differentiation | PAQR5, MMP9, MAF, GLDN, TUBB2A, EPAS1, PDLIM7, |
| | DAB2, ATF5, NRP1, ID2, TM7SF4, CDK5RAP2, ATF5, |
| | MITF, SERPINE2, BCL6, IGFBP3, MCOLN3, NGEF, IGFBP3, |
| | EMP1, FN1, FN1, VEGFA, CSPG4, MMP19, NGEF, ID2, |
| | FN1, PRDM1, IL7R, MITF, IL10, VEGFA, MMP14, GPR183, |
| | VEGFA, INA, RASGRF1, BCL6, MDGA1, ALCAM, PDLIM7, |
| | FABP4, CDK5RAP2, NRP1, VEGFA, ID2, NRCAM, CCND1, |
| | DAB2, MCOLN3, DAB2, ALCAM |
| GO: 0001944~vasculature | CD44, HMOX1, MMP2, CSPG4, NRP1, ANXA2P1, VEGFA, |
| development | MMP19, ANXA2P2, NRP1, NRP1, ANXA2P1, VEGFA, |
| | ANXA2P1, NRP2, ZFP36L1, MMP14, VEGFA, VEGFA, |
| | EPAS1 |
| G0: 0002819~regulation | IL7R, BCL6, CD86, IL27RA, IL10, NOD2, IL7R, TNFSF13B, |
| of adaptive immune | TNFSF13B, BCL6 |
| response | |
| GO: 0051241~negative | HMOX1, BCL6, LOC100131909, ANXA2P1, VEGFA, |
| regulation of | ANXA2P2, ANXA2P1, VEGFA, ANXA2P1, BCLS, STATH, |
| multicellular organismal | IL10, VEGFA, N0D2, VEGFA |
| GO: 0051704~multi- | THBD, IFNGR1, CCL5, TLR4, CX3CR1, PTAFR, CD86, |
| organism process | IL27RA, IL2RG, CXCR7, IL10, IRF7, NOD2, TLR4, MMP1, |
| | IFNGR1, GNLY, IFI44, CCL5, NLRC4, IFNGR1, TLR4, |
| | LILRB1, PLA2G4A, PTX3, THBD, CXCR7, TLR4, THBD, |
| | HIPK2, GNLY, CLE7A, CCL5, ICAM1, TRIM22 |
| GO: 0007165~signal | NRP1, CD36, FGD4, ADORA3, MTSS1, RAB38, DAPP1, |
| transduction | IL7R, CD36, PDK4, ADORA3, ARHGAP18, NOD2, EPAS1, |
| | APB81IP, IFNGR1, ARHGAP18, LPAR6, HMOX1, |
| | LOC100131909, EMR2, ARHGAP18, RGL1, CD244, NRP1, |
| | RAB7B, RIN2, THFRSF21, RAB7B, TNFSF138, CSF1R, |
| | CXCR7, SUCNR1, TLR4, FGL2, P2RY14, CD36, CLEC7A, |
| | APBB1IP, TNFRSF21, CSPG4, ARHGEF3, IFNGR1, CCL5, |
| | RAB27B, TLR4, EPS8, IL7R, TNFSF138, CX3CR1, CCR1, |
| | PTAFR, CNIH3, IL2RG, CXCR7, PDK4, IL10, FGL2, RAPH1, |
| | CCL24, GPR183, TLR4, C3AR1, ARHGAP20, RASGRF1, |
| | ALCAM, NRP1, CCL5, RAPH1, FGD4, DOK2, MTSS1, |
| | GPR35, IFHGR1, TLR4, LPXN, ADORA3, CCND1, HIPK2, |
| | APBB1IP, CCL5, CD36, TREM1, ALCAM |
| GO: 0009617~response | THBD, CCL5, CCL5, NLRC4, TLR4, TLR4, PLA2G4A, THBD, |
| to bacterium | PTAFR, IL27RA, TLR4, THBD, IL10, GNLY, CCL5, NOD2, |
| | TLR4, GNLY |
| GO: 0048519~negative | TCF4, NT5E, NRP1, TRPS1, IL7R, DAB2, DDIT4L, CDKN1A, |
| regulation of biological | NOD2, NT5E, ABHD2, HMOX1, LOC100131909, DAB2, |
| process | ATF5, ANXA2P2, ID2, NRP1, TCF4, ANXA2P1, ATF5, |
| | MITF, TNFSF13B, BCL6, ABHD2, ABHD2, IGFBP3, TLR4, |
| | IFI30, TCF4, TRPS1, GLIS3, CD9, PTGS1, IGFBP3, BCL2A1, |
| | FTGS1, FTHL3, HIVEP1, VEGFA, ANXA2P1, ABHD2, CCL5, |
| | TLR4, ANXA2P1, ID2, PRDM1, IL7R, TNFSF13B, TCF4, |
| | C9ORF89, TRF51, MAFB, ABHD2, MITF, IL27RA, TRPS1, |
| | STATH, IL10, VEGFA, MMP14, MAFB, IRF7, TLR4, |
| | ABHD2, VEGFA, TCF4, BCLG, FABP4, A2M, CCL5, NRP1, |
| | VEGFA, TLR4, SKIL, ID2, PTGS1, ARID5B, CCND1, RGS13, |
| | DAB2, TCF4, ADAMDEC1, HIPK2, CCL5, VASH1, DAB2, |
| | SERTAD1 |
| GO: 0050864~regulation | BCL6, IL27RA, IL2RG, IL10, CDKN1A, TNFSF13B, |
| of B cell activation | TNFSF13B, BCL6 |
| GO: 0001525~angiogenesis | HMOX1, CSPG4, ANXA2P1, VEGFA, NRP1, MMP19, |
| | ANXA2P2, NRP1, ANXA2P1, VEGFA, NRP1, ANXA2P1, |
| | NRP2, MMP14, VEGFA, VEGFA, EPAS1 |
| GO: 0048869~cellular | PAQR5, FN1, CD44, NRP1, NRP2, IL7R, DAB2, ZFP36L1, |
| developments process | PAQR5, MMP9, MAF, GLDN, TUBB2A, EPAS1, PDLIM7, |
| | DAB2, ATF5, NRP1, ID2, TM7SF4, CDK5RAP2, ATF5, |
| | MITF, SERPINE2, BCL6, IGFBP3, MCOLN3, CD9, NGEF, |
| | IGFBP3, EMP1, FN1, FN1, VEGFA, CSPG4, MMP19, NGEF, |
| | ID2, FN1, PRDM1, IL7R, MITF, IL10, VEGFA, MMP14, |
| | GPR183, VEGFA, INA, RASGRF1, BCL6, MDGA1, ALCAM, |
| | PDLIM7, FABP4, CDKSRAP2, NRP1, VEGFA, ID2, NRCAM, |
| | CCND1, DAB2, MCOLN3, DAB2, ALCAM |
| GO: 0002694~regulation | BCL6, LOC100131909, HMOX1, IL7R, TNFSF13B, BCL6, |
| of leukocyte activation | TNFSF13B, IL7R, CD86, IL27RA, IL2RG, IL10, CDKN1A |
| GO: 0044243~multicellular | MMP2, MMP19, MMP9, MMP1 |
| organismal catabolic | |
| process | |
| GO: 0030574~collagen | MMP2, MMP19, MMP9, MMP1 |
| cataboltc process | |
| GO: 0016477~cell | FN1, FN1, CD44, MDGA1, NRP1, NRP1, CCL5, CCL5, |
| migration | NRP1, FN1, NRP2, S100A2, NRCAM, ABID5B, IL10, |
| | MMP14, CCL5, CKLF, ICAM1, FN1 |
| GO: 0045595~regulation | NRP1, CD36, CCL5, ID2, TLR4, IL7R, MAFB, IL7R, MITF, |
| of cell differentiation | CD36, CD86, IL2RG, MAF, MAFB, TLR4, PDLIM7, BCL6, |
| | PDLIM7, ATF5, CDK5RAP2, NRP1, CCL5, NRP1, ID2, TLR4, |
| | CDK5RAP2, ATF5, MITF, BCL6, ID2, IGFBP3, NRCAM, |
| | CCND1, TLR4, CD36, CCL5, CD36, IGFBP3 |
| GO: 0032879~regulation | NRP1, VEGFA, CD36, ABHD2, RAB27B, ABHD2, CD36, |
| of localization | IL10, MMP9, VEGFA, ABHD2, NOD2, VEGFA, ABHD2, |
| | HMOX1, BCL6, LOC100131909, NRP1, NRP1, VEGFA, |
| | SERPINE2, PLA2G4A, ABHD2, BCL6, PTX3, IGFBP3, |
| | ABHD2, PTGS1, CD36, CLEC7A, ENPP2, VASH1, CD36, |
| | IGFBP3, PTGS1, ICAM1, PTGS1 |
| GO: 0050729~positive | LOC100131909, TLR4, FABP4, CCL5, CCL5, TLR4, CCL5, |
| regulation of | TLR4, TLR4, PLA2G4A |
| inflammatory response | |
| GO: 0007186~G-protein | LPAR6, C3AR1, LOC100131909, EMR2, ADORA3, CCL5, |
| coupled receptor protein | CCL5, GPR35, CX3CR1, CCR1, ADORA3, PTAFR, CXCR7, |
| signaling pathway | RGS13, CXCR7, SUCNR1, ADORA3, P2RY14, CCL5, |
| | ENPP2, GPR183 |
| GO: 0051270~regulation | ABHD2, HMOX1, BCL6, MRP1, VEGFA, ABHD2, MRP1, |
| of cell motion | NRP1, VEGFA, SERPINE2, BCL6, ABHD2, IGFBP3, ABHD2, |
| | ABHD2, MMP9, VEGFA, ENPP2, VASH1, ABHD2, VEGFA, |
| | IGFBP3, ICAM1 |
| GO: 0048518~positive | TCF4, CD44, NRP1, CD36, FGD4, SLAMF7, MAML3, IL7R, |
| regulation of biological | CD36, PHLDA1, PHLDA1, MAF, MMP9, CDKN1A, NOD2, |
| process | MAML2, EPAS1, PDUM7, LOC100131909, HMOX1, ID2, |
| | NRP1, TCF4, MAML2, PLA2G4A, MITF, BCL6, PTX3, |
| | TNFSF138, IGFBP3, FGD2, TLR4, TCF4, CD36, CLEC7A, |
| | HGEF, GUS3, PTGS1, IGFBP3, PTGS1, VEGFA, ARHGEF3, |
| | FGD2, NGEF, CCL5, ID2, MAML2, TLR4, PRDM1, IL7R, |
| | TNFSF13B, TCF4, MAFB, MITF, CD86, IL27RA, IL2RG, |
| | IL10, MAFB, VEGFA, TLR4, IRF7, VEGFA, MAML2, TCF4, |
| | C3AR1, RASGRF1, BCL6, PDUM7, MAML2, FABP4, NRP1, |
| | CCL5, NLRC4, FGD4, VEGFA, FGD2, TLR4, CRIP2, |
| | PHLDA1, ID2, PTGS1, FGD2, NRCAM, CCND1, TCF4, |
| | HIPK2, CCL5, CD36, ICAM1, SERTAD1 |
| GO: 001816~cytokine | TLB4, FABP4, NLRC4, MAF, TLR4, NOD2, TLR4, TLR4, |
| production | PTAFR |
| GO: 0007610~behavior | C3AR1, RASGRF1, CCL5, CCL5, EPS8, NRP2, CX3CR1, |
| | CCR1, PTAFR, MCOLN3, HIPK2, IL10, MCOLN3, ANKH, |
| | CCL5, ENPP2, CCL24, CKLF |
| GO: 00550878~regulation | THBD, ANXA2P1, CD36, ANXA2P2, ANXA2P1, ANXA2P1, |
| of body fluid levels | THBD, CD36, STATH, THBD, CD36, HMCN1, CD36, CD9, |
| | PAPSS2 |
| GO: 0032496~response | THBO, CCL5, CCL5, TLR4, TLR4, PLA2G4A, THBD, PTAFR, |
| to lipopolysaccharide | TLR4, THBD, IL10, CCL5, TLR4, NOD2 |
| GO: 0002683~negative | IL7R, HMOX1, BCL6, IL27RA, A2M, IL10, NOD2, IL7R, |
| regulation of immune | BCL6 |
| GO: 0051674~localization | FN1, FN1, CD44, MDGA1, NRP1, NRP1, CCL5, CCL5, |
| of cell | NRP1, FN1, NRP2, S100A2, NRCAM, ARID5B, IL10, |
| | MMP14, CCL5, CKLF, ICAM1, FN1 |
| GO: 0048870~cell motility | FN1, FN1, CD44, MDGA1, NRP1, NRP1, CCL5, CCL5, |
| | NRP1, FN1, NRP2, S100A2, NRCAM, ARID5B, IL10, |
| | MMP14, CCL5, CKLF, ICAM1, FN1 |
| GO: 0046903~secretion | HMOX1, LOC100131909, AQP1, ANKA2P1, DAB2, |
| | ANXA2P2, CCL5, CCL5, ANXA2P1, NLRC4, ANXA2P1, |
| | SYTL1, TNFSF138B, TNFSF13B, DAB2, STATH, DAS2, CCL5, |
| | AQP1, DAB2, CKLF |
| GO: 0006928~cell motion | FN1, FN1, CD44, NRP1, CCL5, FN1, MTSS1, NRP2, |
| | S100A2, IL10, MMP14, CKLF, MDGA1, ALCAM, CCL5, |
| | NRP1, NRP1, MTSS1, ARID5B, NRCAM, CCL5, ENPP2, |
| | CD9, ALCAM, ICAM1, FN1 |
| GO: 0048514~blood | HMOX1, CSPG4, ANXA2P1, VEGFA, NRP1, MMP19, |
| vessel morphogenesis | ANXA2P2, NRP1, NRP1, ANXA2P1, VEGFA, ANXA2P1, |
| | NRP2, ZFP36L1, MMP14, VEGFA, VEGFA, EPAS1 |
| GO: 0001501~skeletal | MMP2, ANXA2P1, PDLIM7, ANXA2P1, ANXA2P1, TRPS1, |
| system development | TFAP2A, TRPS1, IGFBP3, ARID5B, STATH, TRPS1, MMP9, |
| | MMP14, ANKH, TRPS1, PAPSS2, IGFBP3, PDLIM7 |
| GO: 0050867~positive | BCL6, LOC100131909, TLR4, TLR4, IL7R, TNFSF13B, |
| regulation of cell | TNFSF13B, BCL6, IL7R, CD86, TLR4, IL2RG, CDKN1A, |
| activation | TLR4 |
| GO: 0030247~polysaccharide | CD44, FN1, FN1, VEGFA, VEGFA, FN1, SERPINE2, PTX3, |
| binding | CLEC7A, VEGFA, NOD2, ENPP2, VEGFA, FN1 |
| GO: 0001871~pattern | CD44, FN1, FN1, VEGFA, VEGFA, FN1, SERPINE2, PTX3, |
| binding | CLEC7A, VEGFA, NOD2, ENPP2, VEGFA, FN1 |
| GO: 0004222~metalloendopeptidase | MMP2, ADAMDEC1, MMP19, ADAMTS15, MMP9, |
| activity | MMP14, MMP1 |
| GO: 0002698~negative | IL7R, HMOX1, BCL6, A2M, IL7R, BCL6 |
| regulation of immune | |
| effector process | |
| GO: 0002698~positive | LOC100131909, VEGFA, FABP4, CCL5, CCL5, TLR4, |
| regulation of response to | VEGFA, TLR4, PLA2G4A, TLR4, CCL5, VEGFA, TLR4, |
| external stimulus | VEGFA |
| GO: 0010033~response | THBD, CD44, CYP1B1, CCL5, ID2, TLR4, EPS8, HSPB7, |
| to organic substance | ME1, PTAFR, CYP1B1, IL10, MMP14, CDKN1A, AQP1, |
| | NOD2, TLR4, HMOX1, AQP1, CYP1B1, A2M, ME1, |
| | FABP4, CCL5, CYP1B1, ID2, TLR84, PLA2G4A, ID2, THBD, |
| | PTGS1, CCND1, THBD, TLR4, CLEC7A, CCL5, PTGS1, |
| | PLIN2, PTGS1 |

TABLE 6
Human monocyte study: pathways significantly enriched among genes upregulated by Probioglat relative to Copaxone ® at 6 hours.
Pathways significantly enriched among genes upregulated by Probioglat relative to GA (mannitol-corrected comparison) at 6 hours:

| | | Fold | | | |
|---|---|---|---|---|---|
| Term | Pvalue | Enrichment | Benjamini | Probesets | Genes |
| GO:0005576~extracellular | 1.59E−09 | 4.042562158 | 2.49E−07 | 213503_X_AT, 210427_X_AT, | ANXA2P1, ANXA2P1, CD40, PLAUR, CD40, GHRL, |
| region | | | | 205153_S_AT, 214866_AT, | IL7R, GLIPR1, CCL2, STATH, IL10, FGL2, MMP9, |
| | | | | 215346_AT, 223862_AT, 226218_AT, | MMP1, ADAM9, COL6A1, CD14, ANXA2P2, |
| | | | | 226142_AT, 216598_S_AT, | SRPX2, ANXA2P1, TNFSF13B, PTX3, EGF, |
| | | | | 206835_AT, 207433_AT, 227265_AT, | ANTXR2, THBD, ADAMDEC1, ICAM1, CCL5, |
| | | | | 203936_S_AT, 204475_AT, | TREM1, ICAM1 |
| | | | | 202381_AT, 213428_S_AT, 201743_AT, | |
| | | | | 208816_X_AT, 205499_AT | |
| | | | | 201590_X_AT, 223502_S_AT, 206157_AT, | |
| | | | | 206254_AT, 228573_AT, | |
| | | | | 203887_S_AT, 206134_AT, | |
| | | | | 202637_S_AT, 1555759_A_AT, | |
| | | | | 215434_AT, 202638_S_AT | |
| GO:0002376~immune | 1.04E−08 | 4.156626506 | 1.53E−05 | 209933_S_AT, 210017_AT, | CD300A, MALT1, CD40, CD40, NFKBIA, VPS33A, |
| system process | | | | 205153_S_AT, 215346_AT, | IL7R, CCL2, IL10, MMP9, SOD2, GPR183, NOD2, |
| | | | | 201502_S_AT, 204590_X_AT, | CD55, KYNU, CD55, SOD2, ADAM9, CD14, |
| | | | | 226218_AT, 216598_S_AT, | TNFSF13B, PTX3, SOD2, ICAM1, IFIH1, CCL5, |
| | | | | 207433_AT, 203936_S_AT, 221477_S_AT, | MLF1, KYNU, TREM1, ICAM1 |
| | | | | 205419_AT, 220066_AT, | |
| | | | | 201926_S_AT, 217388_S_AT, | |
| | | | | 1555950_A_AT, 215223_S_AT, | |
| | | | | 202381_AT, 201743_AT, 223502_S_AT, | |
| | | | | 206157_AT, 216841_S_AT, | |
| | | | | 202637_S_AT, 219209_AT, | |
| | | | | 1555759_A_AT, 204784_S_AT, | |
| | | | | 210663_S_AT, 219434_AT, 202638_S_AT | |
| GO:0048583~regulation of | 1.56E−07 | 5.897095846 | 0.000115129 | 213503_X_AT, 210017_AT, | ANXA2P1, MALT1, FABP4, ANXA2P2, ANXA2P1, |
| response to stimulus | | | | 203980_AT, 208816_X_AT, | CD40, NFKBIA, ANXA2P1, CD40, GHRL, IL7R, |
| | | | | 210427_X_AT, 205153_S_AT, | THFSF13B, IL10, ICAM1, CCL5, NOD2, CD55, |
| | | | | 201502_S_AT, 201590_X_AT, | NT5E, CD55, ICAM1 |
| | | | | 215346_AT, 223862_AT, 226218_AT, | |
| | | | | 223502_S_AT, 207433_AT, | |
| | | | | 202637_S_AT, 1555759_A_AT, | |
| | | | | 220066_AT, 201926_S_AT, | |
| | | | | 203939_AT, 1555950_A_AT, 202638_S_AT | |
| GO:0044421~extracellular | 2.87E−06 | 4.664494797 | 0.000225327 | 213428_S_AT, 202381_AT, | COL6A1, ADAM9, ANXA2P1, ANXA2P2, |
| region part | | | | 213503_X_AT, 208816_X_AT, | ANXA2P1, ANXA2P1, GHRL, CCL2, TNFSF13B, |
| | | | | 210427_X_AT, 201590_X_AT, | EGF, THBD, IL10, ICAM1, FGL2, MMP9, CCL5, |
| | | | | 223862_AT, 216598_S_AT, | MMP1, ICAM1 |
| | | | | 223502_S_AT, 206254_AT, | |
| | | | | 203887_S_AT, 207433_AT, | |
| | | | | 202637_S_AT, 227265_AT, | |
| | | | | 203936_S_AT, 1555759_A_AT, | |
| | | | | 204475_AT, 202638_S_AT | |
| GO:0048519~negative | 6.21E−07 | 2.625285194 | 0.000305261 | 213891_S_AT, 203927_AT, | TCF4, NFKBIE, ANXA2P1, MALT1, ANXA2P1, |
| regulation of biological | | | | 213503_X_AT, 210017_AT, | P2RX4, NFKBIA, PRDM1, GHRL, IL7R, TCF4, |
| process | | | | 210427_X_AT, 204088_AT, 201502_S_AT, | CCL2, MAFB, DAB2, STATH, IL10, NOD2, SOD2, |
| | | | | 228964_AT, 223862_AT, | NT5E, ARL6IP5, BIRC3, SOD2, TNFRSF9, ARL6IP5, |
| | | | | 226218_AT, 212387_AT, 216598_S_AT, | FABP4, ANXA2P2, ANXA2P1, CHST11, MITF, |
| | | | | 218559_S_AT, 201278_AT, | THFSF13B, EGF, SOD2, TNFAIP3, ADAMDEC1, |
| | | | | 206835_AT, 207433_AT, 220066_AT, | CCL5, CD9 |
| | | | | 221477_S_AT, 203939_AT, | |
| | | | | 200761_S_AT, 210538_S_AT, | |
| | | | | 215223_S_AT, 207536_S_AT, | |
| | | | | 200760_S_AT, 203980_AT, | |
| | | | | 208816_X_AT, 201590_X_AT, | |
| | | | | 219634_AT, 226066_AT, 223502_S_AT, | |
| | | | | 206254_AT, 216841_S_AT, | |
| | | | | 202644_S_AT, 206134_AT, | |
| | | | | 1555759_A_AT, 201005_AT | |
| GO:0006955~immune | 1.10E−06 | 4.644030668 | 0.000325195 | 209933_S_AT, 210017_AT, 201743_AT, | CD300A, MALT1, CD14, IL7R, CCL2, TNFSF13B, |
| response | | | | 226218_AT, 216598_S_AT, | PTX3, IFIH1, ICAMl, IL10, CCL5, NOD2, GPR183, |
| | | | | 223502_S_AT, 206157_AT, 219209_AT, | KYNU, CD55, KYNU, TREM1, CD55, ICAM1 |
| | | | | 202637_S_AT, 207433_AT, | |
| | | | | 1555759_A_AT, 220066_AT, | |
| | | | | 205419_AT, 210663_S_AT, | |
| | | | | 201926_S_AT, 217388_S_AT, | |
| | | | | 219434_AT, 1555950_A_AT, 202638_S_AT | |
| GO:0002237~response to | 1.01E−06 | 14.53261321 | 0.000370434 | 203887_S_AT, 210017_AT, 201743_AT, | THBD, MALT1, CD14, IL10, NFKBIA, CCL5, NOD2, |
| molecule of bacterial origin | | | | 207433_AT, 201502_S_AT, | CCL2 |
| | | | | 1555759_A_AT, 220066_AT, 216598_S_AT | |
| GO:0005886~plasma | 7.17E−06 | 2.132725656 | 0.000375322 | 219358_S_AT, 204912_AT, | ADAP2, IL10RA, CD300A, ANXA2P1, CD36, |
| membrane | | | | 209933_S_AT, 213503_X_AT, | ANXA2P1, P2RX4, FXYD2, CD40, PLAUR, CD40, |
| | | | | 209555_S_AT, 210427_X_AT, | VPS33A, IL7R, SNX20, SYNJ2, SYNJ2, GPR183, |
| | | | | 204088_AT, 207434_S_AT, | NOD2, CD55, NT5E, CD55, STEAP1, LPAR6, |
| | | | | 205153_S_AT, 214866_AT, 215346_AT, | ADAM9, COL6A1, TNFRSF9, CD14, ANXA2P2, |
| | | | | 204590_X_AT, 226218_AT, | ANXA2P1, IFNGR1, TNFSF13B, SNX20, EGF, |
| | | | | 229045_AT, 216180_S_AT, 212828_AT, | ANTXR2, THBD, ICAM1, PLEKHO1, GPR34, |
| | | | | 205419_AT, 220066_AT, | ITGB5, CD9, TREM1, ICAM1 |
| | | | | 201926_S_AT, 203939_AT, | |
| | | | | 1555950_A_AT, 205542_AT, | |
| | | | | 218589_AT, 202381_AT, 213428_S_AT, | |
| | | | | 207536_S_AT, 201743_AT, | |
| | | | | 208816_X_AT, 201590_X_AT, | |
| | | | | 211676_S_AT, 223502_S_AT, | |
| | | | | 228869_AT, 206254_AT, 228573_AT, | |
| | | | | 203887_S_AT, 202637_S_AT, | |
| | | | | 218223_S_AT, 244434_AT, 201125_S_AT, | |
| | | | | 201005_AT, 219434_AT, 202638_S_AT | |
| GO:0051707~response to | 2.07E−06 | 7.289156627 | 0.000507682 | 203887_S_AT, 210017_AT, 201743_AT, | THBD, MALT1, CD14, IFIH1, IL10, NFKBIA, CCL5, |
| other organism | | | | 219209_AT, 207433_AT, | NOD2, IFNGR1, CCL2, PTX3 |
| | | | | 201502_S_AT, 1555759_A_AT, | |
| | | | | 220066_AT, 211676_S_AT, | |
| | | | | 216598_S_AT, 206157_AT | |
| GO:0050878~regulation of | 2.72E−06 | 12.58047114 | 0.000572071 | 206835_AT, 213503_X_AT, | STATH, ANXA2P1, CD36, THBD, ANXA2P2, |
| body fluid levels | | | | 209555_S_AT, 203887_S_AT, | ANXA2P1, CD40, PLAUR, ANXA2P1, CD40, CD9 |
| | | | | 208816_X_AT, 210427_X_AT, | |
| | | | | 205153_S_AT, 214866_AT, | |
| | | | | 201590_X_AT, 215346_AT, 201005_AT | |
| GO:0032879~regulation of | 3.74E−06 | 4.52842902 | 0.000688621 | 202381_AT, 203927_AT, 209555_S_AT, | ADAM9, NFKBIE, CD36, ARL6IP5, RAB27B, |
| localization | | | | 200760_S_AT, 228708_AT, | P2RX4, CD40, NFKBIA, CD40, GHRL, PTX3, EGF, |
| | | | | 204088_AT, 205153_S_AT, 201502_S_AT, | IL10, ICAM1, MMP9, NOD2, ARL6IP5, ICAM1 |
| | | | | 215346_AT, 223862_AT, | |
| | | | | 206157_AT, 206254_AT, 207433_AT, | |
| | | | | 202637_S_AT, 203936_S_AT, | |
| | | | | 220066_AT, 200761_S_AT, 202638_S_AT | |
| GO:0032496~response to | 6.46E−06 | 14.75060241 | 0.000865084 | 203887_S_AT, 201743_AT, | THBD, CD14, IL10, NFKBIA, CCL5, NOD2, CCL2 |
| lipopolysaccharide | | | | 207433_AT, 201502_S_AT, | |
| | | | | 1555759_A_AT, 220066_AT, 216598_S_AT | |
| GO:0060089~molecular | 5.73E−06 | 2.807496252 | 0.000876051 | 204912_AT, 209933_S_AT, 209555_S_AT, | IL10RA, CD300A, CD36, MALT1, P2RX4, CD40, |
| transducer activity | | | | 210017_AT, 204088_AT, | CD40, PLAUR, GHRL, IL7R, CCL2, GPR183, |
| | | | | 205153_S_AT, 215346_AT, 214866_AT, | LPAR6, TNFRSF9, CD14, SRA1, IFNGR1, EGF, |
| | | | | 223862_AT, 226218_AT, | ANTXR2, THBD, ICAM1, CCL5, ITGB5, GPR34, |
| | | | | 216598_S_AT, 205419_AT, 218589_AT, | TREM1, ICAM1 |
| | | | | 207536_S_AT, 201743_AT, | |
| | | | | 224130_S_AT, 211676_S_AT, | |
| | | | | 206254_AT, 228573_AT, | |
| | | | | 203887_S_AT, 202637_S_AT, | |
| | | | | 1555759_A_AT, 201125_S_AT, | |
| | | | | 244434_AT, 219434_AT, 202638_S_AT | |
| GO:004871~signal | 5.73E−06 | 2.807496252 | 0.000876051 | 204912_AT, 209933_S_AT, 209555_S_AT, | IL10RA, CD300A, CD36, MALT1, P2RX4, CD40, |
| transducer activity | | | | 210017_AT, 204088_AT, | CD40, PLAUR, GHRL, IL7R, CCL2, GPR183, |
| | | | | 205153_S_AT, 215346_AT, 214866_AT, | LPAR6, TNFRSF9, CD14, SRA1, IFNGR1, EGF, |
| | | | | 223862_AT, 226218_AT, | ANTXR2, THBD, ICAM1, CCL5, ITGB5, GPR34, |
| | | | | 216598_S_AT, 205419_AT, 218589_AT, | TREM1, ICAM1 |
| | | | | 207536_S_AT, 201743_AT, | |
| | | | | 224130_S_AT, 211676_S_AT, | |
| | | | | 206254_AT, 228573_AT, | |
| | | | | 203887_S_AT, 202637_S_AT, | |
| | | | | 1555759_A_AT, 201125_S_AT, | |
| | | | | 244434_AT, 219434_AT, 202638_S_AT | |
| GO:0065007~biological | 5.36E−06 | 1.477358354 | 0.000877763 | 219358_S_AT, 213891_S_AT, | ADAP2, TCF4, CD36, MALT1, P2RX4, NFE2L3, |
| regulation | | | | 209555_S_AT, 210017_AT, | NFKBIA, VPS33A, DAB2, AGPAT9, MMP9, SOD2, |
| | | | | 204088_AT, 236471_AT, 201502_S_AT, | NOD2, NT5E, CD55, ARHGAP18, BIRC3, SOD2, |
| | | | | 204590_X_AT, 201278_AT, | LPAR6, ADAM9, TNFRSF9, ARL6IP5, ARHGAP18, |
| | | | | 224480_S_AT, 203936_S_AT, | ANXA2P2, BID, ANXA2P1, NFE2L3, CHST11, |
| | | | | 221477_S_AT, 220066_AT, | MITF, PTX3, TNFSF13B, EGF, MXD1, SOD2, |
| | | | | 203939_AT, 1555950_A_AT, | THBD, TNFAIP3, CD9, BID, GLRX3, NFKBIE, |
| | | | | 225166_AT, 210538_S_AT, | ANXA2P1, RAB27B, CD40, ANXA2P1, PLAUR, |
| | | | | 215223_S_AT, 218589_AT, | PRDM1, CD40, GHRL, IL7R, CCL2, TCF4, MAFB, |
| | | | | 202381_AT, 207536_S_AT, | HNRPLL, BID, STATH, IL10, FGL2, GPR183, CD55, |
| | | | | 200760_S_AT, 225171_AT, | ARL6IP5, FABP4, CD14, TNFAIP6, SRA1, MTSS1, |
| | | | | 208816_X_AT, 211725_S_AT, | CYTIP, IFNGR1, CAST, LPXN, ADAMDEC1, IFIH1, |
| | | | | 201590_X_AT, 204702_S_AT, 219634_AT, | ICAM1, CCL5, GPR34, TREM1, ICAM1 |
| | | | | 226066_AT, 206157_AT, | |
| | | | | 223502_S_AT, 206254_AT, | |
| | | | | 226275_AT, 216841_S_AT, | |
| | | | | 203887_S_AT, 202644_S_AT, | |
| | | | | 201005_AT, 204493_AT, | |
| | | | | 209080_X_AT, 203927_AT, | |
| | | | | 213503_X_AT, 228708_AT, | |
| | | | | 205153_S_AT, 210427_X_AT, 214866_AT, | |
| | | | | 228964_AT, 215346_AT, | |
| | | | | 223862_AT, 226218_AT, 216598_S_AT, | |
| | | | | 212387_AT, 218559_S_AT, | |
| | | | | 225386_S_AT, 227143_S_AT, 206835_AT, | |
| | | | | 207433_AT, 227265_AT, | |
| | | | | 205419_AT, 201926_S_AT, 200761_S_AT, | |
| | | | | 203980_AT, 201743_AT, | |
| | | | | 206026_S_AT, 224130_S_AT, | |
| | | | | 203037_S_AT, 209606_AT, | |
| | | | | 211676_S_AT, 208908_S_AT, | |
| | | | | 216250_S_AT, 206134_AT, | |
| | | | | 219209_AT, 202637_S_AT, | |
| | | | | 1555759_A_AT, 244434_AT, | |
| | | | | 219434_AT, 202638_S_AT | |
| GO:0065008~regulation of | 6.39E−06 | 2.871224233 | 0.000941783 | 203927_AT, 213503_X_AT, | NFKBIE, ANXA2P1, CD36, P2RX4, ANXA2P1, |
| biological quality | | | | 209555_S_AT, 204088_AT, | CD40, NFKBIA, CD40, PLAUR, GHRL, IL7R, CCL2, |
| | | | | 210427_X_AT, 205153_S_AT, | BID, DAB2, STATH, IL10, SOD2, CD55, CD55, |
| | | | | 201502_S_AT, 215346_AT, | SOD2, FABP4, ANXA2P2, BID, ANXA2P1, |
| | | | | 214866_AT, 223862_AT, 226218_AT, | TNFSF13B, SOD2, THBD, CCL5, CD9, BID, GLRX3 |
| | | | | 216598_S_AT, 227143_S_AT, | |
| | | | | 201278_AT, 206835_AT, 207433_AT, | |
| | | | | 221477_S_AT, 201926_S_AT, | |
| | | | | 1555950_A_AT, 215223_S_AT, | |
| | | | | 203980_AT, 208816_X_AT, | |
| | | | | 211725_S_AT, 201590_X_AT, | |
| | | | | 223502_S_AT, 216841_S_AT, | |
| | | | | 203887_S_AT, 1555759_A_AT, | |
| | | | | 201005_AT, 204493_AT, 209080_X_AT | |
| GO:0050789~regulation of | 7.87E−06 | 1.49362031 | 0.000966198 | 219358_S_AT, 213891_S_AT, | ADAP2, TCF4, CD36, MALT1, P2RX4, NFE2L3, |
| biological process | | | | 209555_S_AT, 210017_AT, | NFKBIA, VPS33A, DAB2, AGPAT9, MMP9, SOD2, |
| | | | | 204088_AT, 236471_AT, 201502_S_AT, | NOD2, NT5E, CD55, ARHGAP18, BIRC3, SOD2, |
| | | | | 204590_X_AT, 201278_AT, | LPAR6, ADAM9, TNFRSF9, ARL6IP5, ARHGAP18, |
| | | | | 224480_S_AT, 203936_S_AT, | ANXA2P2, BID, ANXA2P1, NFE2L3, CHST11, |
| | | | | 221477_S_AT, 220066_AT, | MITF, PTX3, TNFSF13B, EGF, MXD1, SOD2, |
| | | | | 203939_AT, 1555950_A_AT, | TNFAIP3, CD9, BID, GLRX3, NFKBIE, ANXA2P1, |
| | | | | 225166_AT, 210538_S_AT, | RAB27B, CD40, ANXA2P1, PLAUR, PRDM1, |
| | | | | 215223_S_AT, 218589_AT, | CD40, GHRL, IL7R, CCL2, TCF4, MAFB, HNRPLL, |
| | | | | 202381_AT, 207536_S_AT, | BID, STATH, IL10, FGL2, GPR183, CD55, ARL6IP5, |
| | | | | 200760_S_AT, 225171_AT, | FABP4, CD14, TNFAIP6, SRA1, MTSS1, CYTIP, |
| | | | | 208816_X_AT, 211725_S_AT, | IFNGR1, LPXN, ADAMDEC1, IFIH1, ICAM1, CCL5, |
| | | | | 201590_X_AT, 204702_S_AT, 219634_AT, | GPR34, TREM1, ICAM1 |
| | | | | 226066_AT, 206157_AT, | |
| | | | | 223502_S_AT, 206254_AT, | |
| | | | | 226275_AT, 216841_S_AT, | |
| | | | | 202644_S_AT, 201005_AT, 204493_AT, | |
| | | | | 209080_X_AT, 203927_AT, | |
| | | | | 213503_X_AT, 228708_AT, | |
| | | | | 205153_S_AT, 210427_X_AT, | |
| | | | | 214866_AT, 228964_AT, 215346_AT, | |
| | | | | 223862_AT, 226218_AT, | |
| | | | | 216598_S_AT, 212387_AT, | |
| | | | | 218559_S_AT, 225386_S_AT, | |
| | | | | 227143_S_AT, 206835_AT, 207433_AT, | |
| | | | | 227265_AT, 205419_AT, | |
| | | | | 201926_S_AT, 200761_S_AT, | |
| | | | | 203980_AT, 201743_AT, | |
| | | | | 206026_S_AT, 224130_S_AT, | |
| | | | | 203037_S_AT, 209606_AT, | |
| | | | | 211676_S_AT, 216250_S_AT, | |
| | | | | 206134_AT, 219209_AT, | |
| | | | | 202637_S_AT, 1555759_A_AT, | |
| | | | | 244434_AT, 219434_AT, 202638_S_AT | |
| GO:0051049~regulation of | 8.87E−06 | 5.49711891 | 0.00100533 | 203927_AT, 202381_AT, 200760_S_AT, | NFKBIE, ADAM9, ARL6IP5, RAB27B, CD40, |
| transport | | | | 228708_AT, 205153_S_AT, | P2RX4, CD40, NFKBIA, GHRL, PTX3, EGF, IL10, |
| | | | | 204088_AT, 215346_AT, 201502_S_AT, | NOD2, ARL6IP5 |
| | | | | 223862_AT, 206157_AT, | |
| | | | | 206254_AT, 207433_AT, | |
| | | | | 220066_AT, 200761_S_AT | |
| GO:0004872~receptor | 4.04E−06 | 3.357899833 | 0.001236866 | 218589_AT, 204912_AT, | LPAR6, IL10RA, CD300A, TNFRSF9, CD36, CD14, |
| activity | | | | 209933_S_AT, 207536_S_AT, | P2RX4, CD40, PLAUR, SRA1, CD40, GHRL, |
| | | | | 209555_S_AT, 201743_AT, 204088_AT, | IFNGR1, IL7R, ANTXR2, THBD, ICAM1, GPR183, |
| | | | | 205153_S_AT, 214866_AT, | GPR34, ITGB5, TREM1, ICAM1 |
| | | | | 224130_S_AT, 215346_AT, 223862_AT, | |
| | | | | 211676_S_AT, 226218_AT, | |
| | | | | 228573_AT, 203887_S_AT, 202637_S_AT, | |
| | | | | 205419_AT, 244434_AT, | |
| | | | | 201125_S_AT, 219434_AT, 202638_S_AT | |
| GO:0009611~response to | 1.27E−05 | 4.789156627 | 0.001249473 | 215223_S_AT, 209555_S_AT, | SOD2, CD36, CD14, CD40, CD40, TNFAIP6, |
| wounding | | | | 201743_AT, 205153_S_AT, | PLAUR, CCL2, PTX3, SOD2, THBD, 1L10, CCL5, |
| | | | | 215346_AT, 206026_S_AT, 214866_AT, | SOD2, CD9, CD55, CD55 |
| | | | | 216598_S_AT, 206157_AT, | |
| | | | | 216841_S_AT, 203887_S_AT, | |
| | | | | 207433_AT, 1555759_A_AT, | |
| | | | | 221477_S_AT, 201005_AT, | |
| | | | | 201926_S_AT, 1555950_A_AT | |
| hsa04060: Cytokine- | 1.73E−05 | 6.212389381 | 0.001276815 | 204912_AT, 207536_S_AT, 207433_AT, | IL10RA, TNFRSF9, IL10, CD40, CD40, CCL5, |
| cytokine receptor | | | | 205153_S_AT, 215346_AT, | IFNGR1, IL7R, CCL2, TNFSF13B, EGF |
| interaction | | | | 1555759_A_AT, 211676_S_AT, | |
| | | | | 226218_AT, 216598_S_AT, | |
| | | | | 223502_S_AT, 206254_AT | |
| GO:0002682~regulation of | 1.23E−05 | 5.312341804 | 0.001290482 | 210017_AT, 205153_S_AT, 215346_AT, | MALT1, CD40, CD40, NFKBIA, IL7R, MITF, |
| immune system process | | | | 201502_S_AT, 226218_AT, | TNFSF13B, MAFB, IL10, ICAM1, CCL5, NOD2, |
| | | | | 226066_AT, 223502_S_AT, | CD55, CD55, ICAM1 |
| | | | | 218559_S_AT, 207433_AT, | |
| | | | | 202637_S_AT, 1555759_A_AT, | |
| | | | | 220066_AT, 201926_S_AT, | |
| | | | | 1555950_A_AT, 202638_S_AT | |
| GO:0048518~positive | 1.62E−05 | 2.290466213 | 0.001326312 | 213891_S_AT, 209555_S_AT, | TCF4, CD36, MALT1, P2RX4, CD40, NFKBIA, |
| regulation of biological | | | | 210017_AT, 204088_AT, | CD40, PRDM1, GHRL, IL7R, TCF4, CCL2, MAFB, |
| process | | | | 205153_S_AT, 201502_S_AT, 215346_AT, | HNRPLL, BID, IL10, MMP9, NOD2, SOD2, CD55, |
| | | | | 228964_AT, 223862_AT, | CD55, SOD2, ADAM9, TNFRSF9, FABP4, CD14, |
| | | | | 226218_AT, 212387_AT, | BID, SRA1, MITF, TNFSF13B, PTX3, EGF, SOD2, |
| | | | | 216598_S_AT, 218559_S_AT, | ICAM1, CCL5, BID, ICAM1 |
| | | | | 225386_S_AT, 227143_S_AT, | |
| | | | | 207433_AT, 203936_S_AT, | |
| | | | | 220066_AT, 221477_S_AT, | |
| | | | | 201926_S_AT, 1555950_A_AT, | |
| | | | | 215223_S_AT, 202381_AT, 207536_S_AT, | |
| | | | | 203980_AT, 201743_AT, | |
| | | | | 211725_S_AT, 224130_S_AT, | |
| | | | | 226066_AT, 223502_S_AT, | |
| | | | | 206157_AT, 206254_AT, | |
| | | | | 216841_S_AT, 202637_S_AT, | |
| | | | | 1555759_A_AT, 204493_AT, 202638_S_AT | |
| GO:0051239~regulation of | 1.72E−05 | 3.498329255 | 0.001330564 | 213503_X_AT, 210017_AT, 201743_AT, | ANXA2P1, MALT1, CD14, ANXA2P2, P2RX4, |
| multicellular organismal | | | | 208816_X_AT, 204088_AT, | ANXA2P1, CD40, NFKBIA, ANXA2P1, CD40, |
| process | | | | 210427_X_AT, 205153_S_AT, | GHRL, IL7R, CCL2, MITF, MAFB, EGF, STATH, |
| | | | | 201502_S_AT, 201590_X_AT, | IL10, CCL5, NOD2 |
| | | | | 215346_AT, 223862_AT, 226218_AT, | |
| | | | | 216598_S_AT, 226066_AT, | |
| | | | | 218559_S_AT, 206254_AT, | |
| | | | | 206835_AT, 207433_AT, | |
| | | | | 1555759_A_AT, 220066_AT | |
| GO:0060341~regulation of | 1.61E−05 | 7.836801752 | 0.001398752 | 203927_AT, 202381_AT, 207433_AT, | NFKBIE, ADAM9, IL10, RAB27B, CD40, NFKBIA, |
| cellular localization | | | | 228708_AT, 205153_S_AT, | CD40, NOD2, GHRL, EGF |
| | | | | 201502_S_AT, 215346_AT, 220066_AT, | |
| | | | | 223862_AT, 206254_AT | |
| GO:0051046~regulation of | 1.57E−05 | 9.688408808 | 0.001446037 | 202381_AT, 207433_AT, 228708_AT, | ADAM9, IL10, RAB27B, P2RX4, CD40, CD40, |
| secretion | | | | 204088_AT, 205153_S_AT, | NOD2, GHRL, EGF |
| | | | | 215346_AT, 220066_AT, | |
| | | | | 223862_AT, 206254_AT | |
| GO:0002822~regulation of | 3.26E−05 | 15.80421687 | 0.001846658 | 210017_AT, 207433_AT, 205153_S_AT, | MALT1, IL10, CD40, CD40, NOD2, IL7R, |
| adaptive immune response | | | | 215346_AT, 220066_AT, | TNFSF13B |
| based on somatic | | | | 226218_AT, 223502_S_AT | |
| recombination of immune | | | | | |
| receptors built from | | | | | |
| immunoglobulin | | | | | |
| superfamily domains | | | | | |
| GO:0043067~regulation of | 2.93E−05 | 3.182057087 | 0.001877378 | 215223_S_AT, 202381_AT, 207536_S_AT, | SOD2, ADAM9, TNFRSF9, MALT1, P2RX4, BID, |
| programmed cell death | | | | 210017_AT, 204088_AT, | NFKBIA, GHRL, CHST11, CCL2, MITF, TNFSF13B, |
| | | | | 211725_S_AT, 201502_S_AT, | BID, SOD2, TNFAIP3, IFIH1, IL10, MMP9, SOD2, |
| | | | | 223862_AT, 219634_AT, | NOD2, BID, BIRC3 |
| | | | | 216598_S_AT, 226066_AT, | |
| | | | | 223502_S_AT, 227143_S_AT, | |
| | | | | 216841_S_AT, 202644_S_AT, | |
| | | | | 219209_AT, 207433_AT, | |
| | | | | 203936_S_AT, 221477_S_AT, | |
| | | | | 220066_AT, 204493_AT, 210538_S_AT | |
| GO:0010941~regulation of | 3.06E−05 | 3.171414756 | 0.001878077 | 215223_S_AT, 202381_AT, 207536_S_AT, | SOD2, ADAM9, TNFRSF9, MALT1, P2RX4, BID, |
| cell death | | | | 210017_AT, 204088_AT, | NFKBIA, GHRL, CHST11, CCL2, MITF, TNFSF13B, |
| | | | | 211725_S_AT, 201502_S_AT, | BID, SOD2, TNFAIP3, IFIH1, IL10, MMP9, SOD2, |
| | | | | 223862_AT, 219634_AT, | NOD2, BID, BIRC3 |
| | | | | 216598_S_AT, 226066_AT, | |
| | | | | 223502_S_AT, 227143_S_AT, | |
| | | | | 216841_S_AT, 202644_S_AT, | |
| | | | | 219209_AT, 207433_AT, | |
| | | | | 203936_S_AT, 221477_S_AT, | |
| | | | | 220066_AT, 204493_AT, 210538_S_AT | |
| GO:0009617~response to | 3.20E−05 | 8.689603776 | 0.001886839 | 203887_S_AT, 210017_AT, 201743_AT, | THBD, MALT1, CD14, IL10, NFKBIA, CCL5, NOD2, |
| bacterium | | | | 207433_AT, 201502_S_AT, | CCL2 |
| | | | | 1555759_A_AT, 220066_AT, 216598_S_AT | |
| GO:0042981~regulation of | 2.57E−05 | 3.21441699 | 0.001895789 | 215223_S_AT, 202381_AT, 207536_S_AT, | SOD2, ADAM9, TNFRSF9, MALT1, P2RX4, BID, |
| apoptosis | | | | 210017_AT, 204088_AT, | NFKBIA, GHRL, CHST11, CCL2, MITF, TNFSF13B, |
| | | | | 211725_S_AT, 201502_S_AT, | BID, SOD2, TNFAIP3, IFIH1, IL10, MMP9, SOD2, |
| | | | | 223862_AT, 219634_AT, | NOD2, BID, BIRC3 |
| | | | | 216598_S_AT, 226066_AT, | |
| | | | | 223502_S_AT, 227143_S_AT, | |
| | | | | 216841_S_AT, 202644_S_AT, | |
| | | | | 219209_AT, 207433_AT, | |
| | | | | 203936_S_AT, 221477_S_AT, | |
| | | | | 220066_AT, 204493_AT, 210538_S_AT | |
| GO:0050776~regulation of | 2.88E−05 | 7.238572611 | 0.001926271 | 210017_AT, 202637_S_AT, | MALT1, ICAM1, IL10, CD40, NFKBIA, CD40, |
| immune response | | | | 207433_AT, 205153_S_AT, | NOD2, CD55, IL7R, TNFSF13B, ICAM1, CD55 |
| | | | | 201502_S_AT, 215346_AT, 220066_AT, | |
| | | | | 201926_S_AT, 226218_AT, | |
| | | | | 223502_S_AT, 202638_S_AT, 1555950_A_AT | |
| GO:0048513~organ | 2.80E−05 | 2.616968956 | 0.001962303 | 219358_S_AT, 213503_X_AT, | ADAP2, ANXA2P1, MALT1, ANXA2P1, PRDM1, |
| development | | | | 210017_AT, 210427_X_AT, | PLAUR, GHRL, VPS33A, IL7R, CCL2, MAFB, |
| | | | | 228964_AT, 214866_AT, 223862_AT, | STATH, TGM5, IL10, MMP9, SOD2, GPR183, |
| | | | | 204590_X_AT, 226218_AT, | CRYBB2, SOD2, ADAM9, ANXA2P2, ANXA2P1, |
| | | | | 216598_S_AT, 218559_S_AT, | CHST11, MITF, EGF, SOD2, MLF1 |
| | | | | 206835_AT, 207911_S_AT, | |
| | | | | 207433_AT, 203936_S_AT, | |
| | | | | 221477_S_AT, 205419_AT, | |
| | | | | 206777_S_AT, 215223_S_AT, | |
| | | | | 202381_AT, 208816_X_AT, | |
| | | | | 201590_X_AT, 219634_AT, 226066_AT, | |
| | | | | 206254_AT, 216841_S_AT, 204784_S_AT | |
| GO:0002819~regulation of | 3.69E−05 | 15.41874816 | 0.002010277 | 210017_AT, 207433_AT, 205153_S_AT, | MALT1, IL10, CD40, CD40, NOD2, IL7R, |
| adaptive immune response | | | | 215346_AT, 220066_AT, | TNFSF13B |
| | | | | 226218_AT, 223502_S_AT | |
| GO:0009605~response to | 4.27E−05 | 3.433366869 | 0.002243592 | 215223_S_AT, 209555_S_AT, | SOD2, CD36, CD14, CD40, PLAUR, CD40, |
| external stimulus | | | | 201743_AT, 205153_S_AT, | TNFAIP6, GHRL, CCL2, PTX3, SOD2, THBD, IL10, |
| | | | | 214866_AT, 215346_AT, 206026_S_AT, | CCL5, SOD2, NOD2, KYNU, CD9, CD55, KYNU, |
| | | | | 223862_AT, 216598_S_AT, | CD55 |
| | | | | 206157_AT, 216841_S_AT, | |
| | | | | 203887_S_AT, 207433_AT, | |
| | | | | 1555759_A_AT, 221477_S_AT, | |
| | | | | 220066_AT, 210663_S_AT, | |
| | | | | 201005_AT, 201926_S_AT, | |
| | | | | 217388_S_AT, 1555950_A_AT | |
| GO:0045428~regulation of | 4.62E−05 | 23.94578313 | 0.002345211 | 215223_S_AT, 216841_S_AT, | SOD2, SOD2, ICAM1, IL10, P2RX4, SOD2, ICAM1, |
| nitric oxide biosynthetic | | | | 202637_S_AT, 207433_AT, | PTX3 |
| process | | | | 204088_AT, 221477_S_AT, | |
| | | | | 202638_S_AT, 206157_AT | |
| GO:0051223~regulation of | 5.39E−05 | 10.2434739 | 0.002644616 | 203927_AT, 202381_AT, 207433_AT, | NFKBIE, ADAM9, IL10, CD40, NFKBIA, CD40, |
| protein transport | | | | 205153_S_AT, 201502_S_AT, | NOD2, EGF |
| | | | | 215346_AT, 220066_AT, 206254_AT | |
| GO:0009607~response to | 5.71E−05 | 4.995585791 | 0.002709622 | 203887_S_AT, 210017_AT, 201743_AT, | THBD, MALT1, CD14, IFH1, IL10, NFKBIA, CCL5, |
| biotic stimulus | | | | 219209_AT, 207433_AT, | NOD2, IFNGR1, CCL2, PTX3 |
| | | | | 201502_S_AT, 1555759_A_AT, | |
| | | | | 220066_AT, 211676_S_AT, | |
| | | | | 216598_S_AT, 206157_AT | |
| GO:0005615~extracellular | 7.88E−05 | 4.831639194 | 0.00308733 | 202381_AT, 203887_S_AT, 202637_S_AT, | ADAM9, THBD, ICAM1, IL10, FGL2, MMP9, |
| space | | | | 207433_AT, 227265_AT, | CCL5, GHRL, CCL2, TNFSF13B, ICAM1, EGF |
| | | | | 203936_S_AT, 1555759_A_AT, | |
| | | | | 223862_AT, 216598_S_AT, | |
| | | | | 223502_S_AT, 202638_S_AT, 206254_AT | |
| GO:0051241~negative | 7.32E−05 | 9.704343691 | 0.0033662 | 206835_AT, 213503_X_AT, 208816_X_AT, | STATH, ANXA2P1, ANXA2P2, IL10, P2RX4, |
| regulation of multicellular | | | | 207433_AT, 204088_AT, | ANXA2P1, ANXA2P1, NOD2, GHRL |
| organismal process | | | | 210427_X_AT, 201590_X_AT, | |
| | | | | 220066_AT, 223862_AT | |
| GO:0032101~regulation of | 8.47E−05 | 9.455514365 | 0.00366669 | 213503_X_AT, 203980_AT, | ANXA2P1, FABP4, ANXA2P2, IL10, ANXA2P1, |
| response to external | | | | 208816_X_AT, 207433_AT, | ANXA2P1, CCL5, GHRL, NT5E |
| stimulus | | | | 210427_X_AT, 201590_X_AT, | |
| | | | | 1555759_A_AT, 223862_AT, 203939 AT | |
| GO:0070201~regulation of | 8.47E−05 | 9.455514365 | 0.00366669 | 203927_AT, 202381_AT, 207433_AT, | NFKBIE, ADAM9, IL10, CD40, NFKBIA, CD40, |
| establishment of protein | | | | 205153_S_AT, 201502_S_AT, | NOD2, EGF |
| localization | | | | 215346_AT, 220066_AT, 206254_AT | |
| GO:0050793~regulation of | 8.33E−05 | 3.669304082 | 0.003713809 | 215223_S_AT, 202381_AT, | SOD2, ADAM9, CD36, CD40, NFKBIA, CD40, |
| developmental process | | | | 209555_S_AT, 205153_S_AT, | GHRL, VPS33A, IL7R, MITF, MAFB, EGF, SOD2, |
| | | | | 201502_S_AT, 215346_AT, 223862_AT, | STATH, IL10, CCL5, SOD2 |
| | | | | 204590_X_AT, 226218_AT, | |
| | | | | 226066_AT, 218559_S_AT, 206254_AT, | |
| | | | | 216841_S_AT, 206835_AT, | |
| | | | | 207433_AT, 1555759_A_AT, 221477_S_AT | |
| GO:0048522~positive | 9.67E−05 | 2.22534329 | 0.00406592 | 213891_S_AT, 209555_S_AT, | TCF4, CD36, MALT1, P2RX4, CD40, NFKBIA, |
| regulation of cellular | | | | 210017_AT, 204088_AT, | CD40, GHRL, IL7R, TCF4, CCL2, MAFB, HNRPLL, |
| process | | | | 205153_S_AT, 201502_S_AT, 215346_AT, | BID, IL10, MMP9, NOD2, SOD2, SOD2, ADAM9, |
| | | | | 223862_AT, 226218_AT, | TNFRSF9, FABP4, BID, SRA1, MITF, TNFSF13B, |
| | | | | 212387_AT, 216598_S_AT, | EGF, PTX3, SOD2, ICAM1, CCL5, BID, ICAM1 |
| | | | | 218559_S_AT, 225386_S_AT, | |
| | | | | 227143_S_AT, 207433_AT, | |
| | | | | 203936_S_AT, 220066_AT, | |
| | | | | 221477_S_AT, 215223_S_AT, | |
| | | | | 202381_AT, 207536_S_AT, | |
| | | | | 203980_AT, 211725_S_AT, | |
| | | | | 224130_S_AT, 226066_AT, | |
| | | | | 223502_S_AT, 206254_AT, | |
| | | | | 206157_AT, 216841_S_AT, | |
| | | | | 202637_S_AT, 1555759_A_AT, | |
| | | | | 204493_AT, 202638_S_AT | |
| GO:0032501~multicellular | 0.000104285 | 1.860570435 | 0.004260994 | 219358_S_AT, 213503_X_AT, | ADAP2, ANXA2P1, CD36, MALT1, ANXA2P1, |
| organismal process | | | | 209555_S_AT, 210017_AT, | P2RX4, CD40, PLAUR, PRDM1, CD40, VPS33A, |
| | | | | 210427_X_AT, 204088_AT, 205153_S_AT, | GHRL, IL7R, CCL2, MAFB, DAB2, STATH, TGM5, |
| | | | | 214866_AT, 228964_AT, | IL10, MMP9, GPR183, NOD2, SOD2, MMP1, |
| | | | | 215346_AT, 204590_X_AT, 223862_AT, | BIRC3, CRYBB2, SOD2, ADAM9, FABP4, |
| | | | | 226218_AT, 216598_S_AT, | ANXA2P2, ANXA2P1, CHST11, MITF, EGF, |
| | | | | 218559_S_AT, 201278_AT, 206835_AT, | AKR1C2, MXD1, SOD2, THBD, MLF1, CD9 |
| | | | | 207911_S_AT, 207433_AT, | |
| | | | | 203936_S_AT, 205419_AT, 220066_AT, | |
| | | | | 221477_S_AT, 204475_AT, | |
| | | | | 210538_S_AT, 206777_S_AT, | |
| | | | | 215223_S_AT, 202381_AT, | |
| | | | | 203980_AT, 208816_X_AT, 201590_X_AT, | |
| | | | | 219634_AT, 226066_AT, | |
| | | | | 206254_AT, 211653_X_AT, | |
| | | | | 226275_AT, 216841_S_AT, | |
| | | | | 203887_S_AT, 204784_S_AT, 201005_AT | |
| GO:0007166-cell surface | 0.000113804 | 2.856183771 | 0.004523702 | 218589_AT, 202381_AT, 210017_AT, | LPAR6, ADAM9, MALT1, CD14, CD40, NFKBIA, |
| receptor linked signal | | | | 201743_AT, 205153_S_AT, | CD40, GHRL, MTSS1, IL7R, CCL2, MITF, EGF, |
| transduction | | | | 201502_S_AT, 215346_AT, 223862_AT, | ADAMDEC1, CCL5, GPR183, GPR34, ITGB5, |
| | | | | 203037_S_AT, 226218_AT, | BIRC3 |
| | | | | 216598_S_AT, 226066_AT, | |
| | | | | 206254_AT, 206134_AT, | |
| | | | | 1555759_A_AT, 205419_AT, | |
| | | | | 244434_AT, 201125_S_AT, 210538_S_AT | |
| GO:0006916~anti- | 0.000124449 | 5.889770261 | 0.004815965 | 215223_S_AT, 216841_S_AT, | SOD2, SOD2, TNFAIP3, MALT1, IL10, NFKBIA, |
| apoptosis | | | | 202644_S_AT, 210017_AT, | SOD2, GHRL, CCL2, TNFSF13B, BIRC3 |
| | | | | 207433_AT, 201502_S_AT, | |
| | | | | 221477_S_AT, 223862_AT, | |
| | | | | 216598_S_AT, 223502_S_AT, 210538_S_AT | |
| GO:0043066~negative | 0.000129722 | 4.527249623 | 0.004891125 | 215223_S_AT, 210017_AT, 201502_S_AT, | SOD2, MALT1, NFKBIA, GHRL, CHST11, CCL2, |
| regulation of apoptosis | | | | 223862_AT, 219634_AT, | MITF, TNFSF13B, SOD2, TNFAIP3, IL10, SOD2, |
| | | | | 216598_S_AT, 226066_AT, | BIRC3 |
| | | | | 223502_S_AT, 216841_S_AT, | |
| | | | | 202644_S_AT, 207433_AT, | |
| | | | | 221477_S_AT, 210538_S_AT | |
| GO:0060548~negative | 0.000147362 | 4.457599629 | 0.005158736 | 215223_S_AT, 210017_AT, 201502_S_AT, | SOD2, MALT1, NFKBIA, GHRL, CHST11, CCL2, |
| regulation of cell death | | | | 223862_AT, 219634_AT, | MITF, TNFSF13B, SOD2, TNFAIP3, IL10, SOD2, |
| | | | | 216598_S_AT, 226066_AT, | BIRC3 |
| | | | | 223502_S_AT, 216841_S_AT, | |
| | | | | 202644_S_AT, 207433_AT, | |
| | | | | 221477_S_AT, 210538_S_AT | |
| GO:0043069~negative | 0.000142772 | 4.474810439 | 0.005247712 | 215223_S_AT, 210017_AT, 201502_S_AT, | SOD2, MALT1, NFKBIA, GHRL, CHST11, CCL2, |
| regulation of programmed | | | | 223862_AT, 219634_AT, | MITF, TNFSF13B, SOD2, TNFAIP3, IL10, SOD2, |
| cell death | | | | 216598_S_AT, 226066_AT, | BIRC3 |
| | | | | 223502_S_AT, 216841_S_AT, | |
| | | | | 202644_S_AT, 207433_AT, | |
| | | | | 221477_S_AT, 210538_S_AT | |
| GO:0009893~positive | 0.000147162 | 2.926706827 | 0.005277077 | 213891_S_AT, 215223_S_AT, | TCF4, SOD2, ADAM9, P2RX4, CD40, NFKBIA, |
| regulation of metabolic | | | | 202381_AT, 204088_AT, | SRA1, CD40, PRDM1, TCF4, MITF, TNFSF13B, |
| process | | | | 205153_S_AT, 201502_S_AT, | MAFB, EGF, PTX3, HNRPLL, SOD2, ICAM1, IL10, |
| | | | | 224130_S_AT, 215346_AT, | SOD2, NOD2, ICAM1 |
| | | | | 228964_AT, 212387_AT, 226066_AT, | |
| | | | | 223502_S_AT, 218559_S_AT, | |
| | | | | 206254_AT, 206157_AT, | |
| | | | | 225386_S_AT, 216841_S_AT, | |
| | | | | 202637_S_AT, 207433_AT, | |
| | | | | 221477_S_AT, 220066_AT, 202638_S_AT | |
| GO:0031347~regulation of | 0.000165947 | 8.381024096 | 0.005672844 | 203980_AT, 207433_AT, | FABP4, IL10, NFKBIA, CCL5, NOD2, GHRL, NT5E |
| defense response | | | | 201502_S_AT, 1555759_A_AT, | |
| | | | | 220066_AT, 223862_AT, 203939_AT | |
| GO:0006952~defense | 0.000177009 | 3.963439967 | 0.005912766 | 210017_AT, 201743_AT, 205153_S_AT, | MALT1, CD14, CD40, TNFAIP6, CD40, CCL2, |
| response | | | | 206026_S_AT, 215346_AT, | PTX3, IFIH1, IL10, CCL5, NOD2, KYNU, CD55, |
| | | | | 216598_S_AT, 206157_AT, | KYNU, CD55 |
| | | | | 219209_AT, 207433_AT, | |
| | | | | 1555759_A_AT, 220066_AT, | |
| | | | | 210663_S_AT, 201926_S_AT, | |
| | | | | 217388_S_AT, 1555950_A_AT | |
| GO:0050708~regulation of | 0.000186347 | 16.99378158 | 0.00608588 | 202381_AT, 207433_AT, 205153_S_AT, | ADAM9, IL10, CD40, CD40, NOD2, EGF |
| protein secretion | | | | 215346_AT, 220066_AT, 206254_AT | |
| GO:0051704~multi- | 0.000215223 | 3.337240364 | 0.006727739 | 210017_AT, 201743_AT, 201502_S_AT, | MALT1, CD14, NFKBIA, GHRL, IFNGR1, CCL2, |
| organism process | | | | 223862_AT, 211676_S_AT, | PTX3, THBD, ICAM1, IL10, IFIH1, CCL5, NOD2, |
| | | | | 216598_S_AT, 206157_AT, | MMP1, ICAM1 |
| | | | | 203887_S_AT, 202637_S_AT, | |
| | | | | 207433_AT, 219209_AT, 1555759_A_AT, | |
| | | | | 220066_AT, 204475_AT, 202638_S_AT | |
| GO:0032880~regulation of | 0.00021195 | 8.016631744 | 0.006769334 | 203927_AT, 202381_AT, 207433_AT, | NFKBIE, ADAM9, IL10, CD40, NFKBIA, CD40, |
| protein localization | | | | 205153_S_AT, 201502_S_AT, | NOD2, EGF |
| | | | | 215346_AT, 220066_AT, 206254_AT | |
| GO:0002697~regulation of | 0.000233713 | 10.53614458 | 0.007006643 | 210017_AT, 202637_S_AT, 207433_AT, | MALT1, ICAM1, IL10, CD40, CD40, NOD2, IL7R, |
| immune effector process | | | | 205153_S_AT, 215346_AT, | ICAM1 |
| | | | | 220066_AT, 226218_AT, 202638_S_AT | |
| GO:0002706~regulation of | 0.000238915 | 15.96385542 | 0.007019318 | 210017_AT, 207433_AT, 205153_S_AT, | MALT1, IL10, CD40, CD40, NOD2, IL7R |
| lymphocyte mediated | | | | 215346_AT, 220066_AT, 226218_AT | |
| immunity | | | | | |
| GO:0080134~regulation of | 0.000230106 | 5.387801205 | 0.007042074 | 213503_X_AT, 203980_AT, | ANXA2P1, FABP4, ANXA2P2, IL10, ANXA2P1, |
| response to stress | | | | 208816_X_AT, 207433_AT, | NFKBIA, ANXA2P1, CCL5, NOD2, GHRL, NT5E |
| | | | | 210427_X_AT, 201502_S_AT, | |
| | | | | 201590_X_AT, 1555759_A_AT, | |
| | | | | 220066_AT, 223862_AT, 203939_AT | |
| GO:0032502~developmental | 0.000273185 | 1.891259305 | 0.007865563 | 219358_S_AT, 213503_X_AT, | ADAP2, ANXA2P1, MALT1, ANXA2P1, PLAUR, |
| process | | | | 210017_AT, 210427_X_AT, | PRDM1, VPS33A, GHRL, IL7R, CCL2, MAFB, |
| | | | | 214866_AT, 228964_AT, 204590_X_AT, | DAB2, TGM5, STATH, IL10, MMP9, SOD2, |
| | | | | 223862_AT, 226218_AT, | GPR183, SOD2, CRYBB2, ADAM9, FABP4, |
| | | | | 216598_S_AT, 218559_S_AT, | ANXA2P2, ANXA2P1, SRA1, CAST, CHST11, |
| | | | | 201278_AT, 207911_S_AT, | MITF, EGF, MXD1, SOD2, THBD, MLF1, CCL5, |
| | | | | 206835_AT, 207433_AT, 203936_S_AT, | CD9 |
| | | | | 221477_S_AT, 205419_AT, | |
| | | | | 215223_S_AT, 206777_S_AT, | |
| | | | | 202381_AT, 203980_AT, | |
| | | | | 208816_X_AT, 201590_X_AT, | |
| | | | | 224130_S_AT, 208908_S_AT, | |
| | | | | 219634_AT, 226066_AT, 206254_AT, | |
| | | | | 226275_AT, 216841_S_AT, | |
| | | | | 203887_S_AT, 204784_S_AT, | |
| | | | | 1555759_A_AT, 201005_AT | |
| GO:0048584~positive | 0.000283953 | 6.152493184 | 0.008017793 | 210017_AT, 203980_AT, | MALT1, FABP4, NFKBIA, CCL5, NOD2, GHRL, |
| regulation of response to | | | | 201502_S_AT, 1555759_A_AT, | CD55,TNFSF13B, CD55 |
| stimulus | | | | 220066_AT, 223862_AT, | |
| | | | | 201926_S_AT, 223502_S_AT, 1555950_A_AT | |
| GO:0007155~cell adhesion | 0.000307737 | 3.718639263 | 0.008523348 | 209933_S_AT, 213428_S_AT, | CD300A, COL6A1, ADAM9, CD36, TNFAIP6, |
| | | | | 202381_AT, 209555_S_AT, | MTSS1, CCL2, LPXN, ICAM1, CCL5, ITGB5, CD9, |
| | | | | 206026_S_AT, 203037_S_AT, | ICAM1 |
| | | | | 216598_S_AT, 216250_S_AT, | |
| | | | | 202637_S_AT, 1555759_A_AT, | |
| | | | | 201125_S_AT, 201005_AT, 202638_S_AT | |
| GO:0022610~biologieal | 0.000315533 | 3.707734162 | 0.008578876 | 209933_S_AT, 213428_S_AT, | CD300A, COL6A1, ADAM9, CD36, TNFAIP6, |
| adhesion | | | | 202381_AT, 209555_S_AT, | MTSS1, CCL2, LPXN, ICAM1, CCL5, ITGB5, CD9, |
| | | | | 206026_S_AT, 203037_S_AT, | ICAM1 |
| | | | | 216598_S_AT, 216250_S_AT, | |
| | | | | 202637_S_AT, 1555759_A_AT, | |
| | | | | 201125_S_AT, 201005_AT, 202638_S_AT | |
| GO:0002684~positive | 0.000324184 | 6.020654045 | 0.008651889 | 210017_AT, 202637_S_AT, | MALT1, ICAM1, CD40, NFKBIA, CD40, NOD2, |
| regulation of immune | | | | 205153_S_AT, 201502_S_AT, | CD55, IL7R, TNFSF13B, ICAM1, CD55 |
| system process | | | | 215346_AT, 220066_AT, 201926_S_AT, | |
| | | | | 226218_AT, 223502_S_AT, | |
| | | | | 202638_S_AT, 1555950_A_AT | |
| GO:0001775~cell activation | 0.000386538 | 4.990805327 | 0.010124588 | 202381_AT, 210017_AT, 202637_S_AT, | ADAM9, MALT1, ICAM1, IL10, P2RX4, CD40, |
| | | | | 207433_AT, 204088_AT, | CD40, GPR183, CD9, IL7R, ICAM1 |
| | | | | 205153_S_AT, 215346_AT, 205419_AT, | |
| | | | | 201005_AT, 226218_AT, 202638_S_AT | |
| GO:0045429~positive | 0.000407508 | 26.34036145 | 0.010484798 | 215223_S_AT, 216841_S_AT, | SOD2, SOD2, ICAM1, P2RX4, SOD2, ICAM1, |
| regulation of nitric oxide | | | | 202637_S_AT, 204088_AT, | PTX3 |
| biosynthetic process | | | | 221477_S_AT, 202638_S_AT, 206157_AT | |
| GO:0002703~regulation of | 0.00041591 | 13.86334813 | 0.01051636 | 210017_AT, 207433_AT, 205153_S_AT, | MALT1, IL10, CD40, CD40, NOD2, IL7R |
| leukocyte mediated | | | | 215346_AT, 220066_AT, 226218_AT | |
| immunity | | | | | |
| GO:0048856~anatomical | 0.000458417 | 2.018715984 | 0.011389929 | 219358_S_AT, 213503_X_AT, | ADAP2, ANXA2P1, MALT1, ANXA2P1, PRDM1, |
| structure development | | | | 210017_AT, 210427_X_AT, | PLAUR, VPS33A, GHRL, IL7R, CCL2, MAFB, DAB2, |
| | | | | 228964_AT, 214866_AT, 204590_X_AT, | TGM5, STATH, IL10, MMP9, SOD2, GPR183, |
| | | | | 223862_AT, 226218_AT, | SOD2, CRYBB2, ADAM9, ANXA2P2, ANXA2P1, |
| | | | | 216598_S_AT, 212559_S_AT, | CAST, CHST11, MITF, EGF, SOD2, MLF1, CD9 |
| | | | | 201278_AT, 207911_S_AT, | |
| | | | | 206835_AT, 207433_AT, 203936_S_AT, | |
| | | | | 221477_S_AT, 205419_AT, | |
| | | | | 215223_S_AT, 206777_S_AT, | |
| | | | | 202381_AT, 208816_X_AT, | |
| | | | | 201590_X_AT, 208908_S_AT, 219634_AT, | |
| | | | | 226066_AT, 206254_AT, | |
| | | | | 216841_S_AT, 204784_S_AT, 201005_AT | |
| GO:0046903~secretion | 0.000554725 | 5.509095204 | 0.013539046 | 201278_AT, 206835_AT, | DAB2, STATH, ANXA2P1, ANXA2P2, ANXA2P1, |
| | | | | 213503_X_AT, 208816_X_AT, | ANXA2P1, CCL5, VPS33A, GHRL, TNFSF13B |
| | | | | 210427_X_AT, 201590_X_AT, | |
| | | | | 1555759_A_AT, 204590_X_AT, | |
| | | | | 223862_AT, 223502_S_AT | |
| GO:0050727~regulation of | 0.000612986 | 12.54302926 | 0.01470747 | 203980_AT, 207433_AT, 1555759_A_AT, | FABP4, IL10, CCL5, GHRL, NT5E |
| inflammatory response | | | | 223862_AT, 203939_AT | |
| GO:0048731~system | 0.000635394 | 2.065910702 | 0.014997197 | 219358_S_AT, 213503_X_AT, | ADAP2, ANXA2P1, MALT1, ANXA2P1, PRDM1, |
| development | | | | 210017_AT, 210427_X_AT, | PLAUR, GHRL, VPS33A, IL7R, CCL2, MAFB, |
| | | | | 228964_AT, 214866_AT, 223862_AT, | STATH, TGM5, IL10, MMP9, SOD2, GPR183, |
| | | | | 204590_X_AT, 226218_AT, | SOD2, CRYBB2, ADAM9, ANXA2P2, ANXA2P1, |
| | | | | 216598_S_AT, 218559_S_AT, | CHST11, MITF, EGF, SOD2, MLF1, CD9 |
| | | | | 206835_AT, 207911_S_AT, | |
| | | | | 207433_AT, 203936_S_AT, | |
| | | | | 221477_S_AT, 205419_AT, | |
| | | | | 215223_S_AT, 206777_S_AT, | |
| | | | | 202381_AT, 208816_X_AT, | |
| | | | | 201590_X_AT, 219634_AT, 226066_AT, | |
| | | | | 206254_AT, 216841_S_AT, | |
| | | | | 204784_S_AT, 201005_AT | |
| GO:0031226~intrinsic to | 0.000511383 | 2.901401351 | 0.015933223 | 202381_AT, 207536_S_AT, | ADAM9, TNFRSF9, CD36, P2RX4, FXYD2, CD40, |
| plasma membrane | | | | 209555_S_AT, 204088_AT, | CD40, IFNGR1, THBD, ICAM1, GPR183, CD9, |
| | | | | 207434_S_AT, 205153_S_AT, | CD55, ITGB5, GPR34, CD55, STEAP1, ICAM1 |
| | | | | 215346_AT, 211676_S_AT, | |
| | | | | 203887_S_AT, 202637_S_AT, | |
| | | | | 205419_AT, 201005_AT, | |
| | | | | 201926_S_AT, 201125_S_AT, | |
| | | | | 244434_AT, 1555950_A_AT, | |
| | | | | 205542_AT, 202638_S_AT | |
| GO:0051051~negative | 0.00074669 | 8.209982788 | 0.017324877 | 203927_AT, 200760_S_AT, 207433_AT, | NFKBIE, ARL6IP5, IL10, NFKBIA, GHRL, ARL6IP5, |
| regulation of transport | | | | 201502_S_AT, 223862_AT, | EGF |
| | | | | 200761_S_AT, 206254_AT | |
| GO:0051050~positive | 0.000772048 | 6.303676243 | 0.017630829 | 202381_AT, 207433_AT, 204088_AT, | ADAM9, IL10, P2RX4, NFKBIA, NOD2, GHRL, |
| regulation of transport | | | | 201502_S_AT, 220066_AT, | PTX3 |
| | | | | 223862_AT, 206157_AT | |
| GO:0051384~response to | 0.000868651 | 11.45233106 | 0.019513971 | 202381_AT, 203980_AT, 207433_AT, | ADAM9, FABP4, IL10, CCL5, CCL2 |
| glucocorticoid stimulus | | | | 1555759_A_AT, 216598_S_AT | |
| GO:0005625~soluble | 0.000768008 | 4.517896389 | 0.019903205 | 215223_S_AT, 213503_X_AT, | SOD2, ANXA2P1, FABP4, ANXA2P1, ANXA2P1, |
| fraction | | | | 203980_AT, 210427_X_AT, | TNFSF13B, EGF, SOD2, CCL5, SOD2, KYNU, |
| | | | | 201590_X_AT, 223502_S_AT, | GPR34, CD55, KYNU, CD55 |
| | | | | 206254_AT, 216841_S_AT, | |
| | | | | 1555759_A_AT, 221477_S_AT, | |
| | | | | 210663_S_AT, 244434_AT, | |
| | | | | 201926_S_AT, 217388_S_AT, 1555950_A_AT | |
| GO:0031325~positive | 0.000976424 | 2.701575533 | 0.021581183 | 213891_S_AT, 215223_S_AT, | TCF4, SOD2, ADAM9, P2RX4, CD40, NFKBIA, |
| regulation of cellular | | | | 202381_AT, 204088_AT, | SRA1, CD40, TCF4, MITF, MAFB, EGF, PTX3, |
| metabolic process | | | | 205153_S_AT, 201502_S_AT, | HNRPLL, SOD2, ICAM1, IL10, SOD2, NOD2, |
| | | | | 224130_S_AT, 215346_AT, | ICAM1 |
| | | | | 212387_AT, 226066_AT, 218559_S_AT, | |
| | | | | 206254_AT, 206157_AT, | |
| | | | | 225386_S_AT, 216841_S_AT, | |
| | | | | 202637_S_AT, 207433_AT, | |
| | | | | 221477_S_AT, 220066_AT, 202638_S_AT | |
| GO:0050817~coagulation | 0.001021169 | 10.9751506 | 0.022226472 | 209555_S_AT, 203887_S_AT, | CD36, THBD, CD40, PLAUR, CD40, CD9 |
| | | | | 205153_S_AT, 214866_AT, | |
| | | | | 215346_AT, 201005_AT | |
| GO:0007596~blood | 0.001021169 | 10.9751506 | 0.022226472 | 209555_S_AT, 203887_S_AT, | CD36, THBD, CD40, PLAUR, CD40, CD9 |
| coagulation | | | | 205153_S_AT, 214866_AT, | |
| | | | | 215346_AT, 201005_AT | |
| GO:0031960~response to | 0.001104078 | 10.75116794 | 0.023661309 | 202381_AT, 203980_AT, 207433_AT, | ADAM9, FABP4, IL10, CCL5, CCL2 |
| corticosteroid stimulus | | | | 1555759_A_AT, 216598_S_AT | |
| GO:0051047~positive | 0.0001104078 | 10.75116794 | 0.023661309 | 202381_AT, 207433_AT, 204088_AT, | ADAM9, IL10, P2RX4, NOD2, GHRL |
| regulation of secretion | | | | 220066_AT, 223862_AT | |
| GO:0030097~hemopoiesis | 0.001226255 | 4.816523236 | 0.025151654 | 215223_S_AT, 216841_S_AT, | SOD2, SOD2, MALT1, IL10, MMP9, MLF1, SOD2, |
| | | | | 210017_AT, 207433_AT, | GPR183, VPS33A, IL7R |
| | | | | 203936_S_AT, 204784_S_AT, | |
| | | | | 221477_S_AT, 205419_AT, | |
| | | | | 204590_X_AT, 226218_AT | |
| GO:0007275~multicellular | 0.001212038 | 1.856892321 | 0.025215437 | 219358_S_AT, 213503_X_AT, | ADAP2, ANXA2P1, MALT1, ANXA2P1, PLAUR, |
| organismal development | | | | 210017_AT, 210427_X_AT, | PRDM1, VPS33A, GHRL, IL7R, CCL2, MAFB, |
| | | | | 214866_AT, 228964_AT, 204590_X_AT, | DAB2, TGM5, STATH, IL10, MMP9, SOD2, |
| | | | | 223862_AT, 226218_AT, | GPR183, SOD2, CRYBB2, ADAM9, ANXA2P2, |
| | | | | 216598_S_AT, 218559_S_AT, | ANXA2P1, CHST11, MITF, EGF, MXD1, SOD2, |
| | | | | 201278_AT, 207911_S_AT, | THBD, MLF1, CD9 |
| | | | | 206835_AT, 207433_AT, 203936_S_AT, | |
| | | | | 221477_S_AT, 205419_AT, | |
| | | | | 215223_S_AT, 206777_S_AT, | |
| | | | | 202381_AT, 208816_X_AT, | |
| | | | | 201590_X_AT, 219634_AT, 226066_AT, | |
| | | | | 206254_AT, 226275_AT, | |
| | | | | 216841_S_AT, 203887_S_AT, | |
| | | | | 204784_S_AT, 201005_AT | |
| GO:0010033~response to | 0.001204931 | 2.951937059 | 0.025426793 | 202381_AT, 201743_AT, 203980_AT, | ADAM9, CD14, FABP4, MALT1, P2RX4, NFKBIA, |
| organic substance | | | | 210017_AT, 204088_AT, | GHRL, CCL2, THBD, IL10, CCL5, NOD2, KYNU, |
| | | | | 201502_S_AT, 223862_AT, | KYNU |
| | | | | 216598_S_AT, 203887_S_AT, | |
| | | | | 207433_AT, 1555759_A_AT, | |
| | | | | 220066_AT, 210663_S_AT, 217388_S_AT | |
| hsa04621: NOD-like | 0.000725707 | 7.947169811 | 0.026503364 | 202644_S_AT, 201502_S_AT, | TNFAIP3, NFKBIA, CCL5, NOD2, CD2, BIRC3 |
| receptor signaling pathway | | | | 1555759_A_AT, 220066_AT, | |
| | | | | 216598_S_AT, 210538_S_AT | |
| GO:0002252~immune | 0.00136637 | 7.18373434 | 0.027603619 | 202637_S_AT, 220066_AT, | ICAM1, NOD2, CD55, IL7R, TNFSF13B, ICAM1, |
| effector process | | | | 201926_S_AT, 226218_AT, | CD55, PTX3 |
| | | | | 223502_S_AT, 202638_S_AT, | |
| | | | | 1555950_A_AT, 206157_AT | |
| GO:0032680~regulation of | 0.001395324 | 17.56024096 | 0.027800016 | 201743_AT, 207433_AT, | CD14, IL10, NOD2, GHRL |
| tumor necrosis factor | | | | 220066_AT, 223862_AT | |
| production | | | | | |
| GO:0006954~inflammatory | 0.001443579 | 4.682730924 | 0.028365247 | 201743_AT, 207433_AT, 205153_S_AT, | CD14, IL10, CD40, CD40, TNFAIP6, CCL5, CD55, |
| response | | | | 215346_AT, 206026_S_AT, | CCL2, CD55, PTX3 |
| | | | | 1555759_A_AT, 201926_S_AT, | |
| | | | | 216598_S_AT, 1555950_A_AT, | |
| | | | | 206157_AT | |
| GO:0007599~hemostasis | 0.001483225 | 9.939759036 | 0.028750558 | 209555_S_AT, 203887_S_AT, | CD36, THBD, CD40, PLAUR, CD40, CC9 |
| | | | | 205153_S_AT, 214866_AT, | |
| | | | | 215346_AT, 201005_AT | |
| GO:0051173~positive | 0.001639177 | 3.039272475 | 0.03014551 | 213891_S_AT, 215223_S_AT, | TCF4, SOD2, CD40, P2RX4, SRA1, CD40, NFKBIA, |
| regulation of nitrogen | | | | 205153_S_AT, 204088_AT, | TCF4, MITF, PTX3, MAFB, HNRPLL, SOD2, IL10, |
| compound metabolic | | | | 224130_S_AT, 215346_AT, 201502_S_AT, | ICAM1, SOD2, ICAM1 |
| process | | | | 212387_AT, 226066_AT, | |
| | | | | 206157_AT, 218559_S_AT, | |
| | | | | 225386_S_AT, 216841_S_AT, | |
| | | | | 207433_AT, 202637_S_AT, | |
| | | | | 221477_S_AT, 202638_S_AT | |
| GO:0051240~positive | 0.001622601 | 5.463186078 | 0.030221815 | 210017_AT, 201743_AT, | MALT1, CD14, CD40, CD40, CCL5, NOD2, GHRL, |
| regulation of multicellular | | | | 205153_S_AT, 215346_AT, | CCL2 |
| organismal process | | | | 1555759_A_AT, 220066_AT, | |
| | | | | 223862_AT, 216598_S_AT | |
| GO:0042127~regulation of | 0.00158235 | 2.859496441 | 0.0302467 | 215223_S_AT, 207536_S_AT, | SOD2, TNFRSF9, FABP4, CD40, CD40, NFKBIA, |
| cell proliferation | | | | 203980_AT, 205153_S_AT, | GHRL, CHST11, CCL2, MITF, TNFSF13B, EGF, |
| | | | | 215346_AT, 201502_S_AT, 223862_AT, | SOD2, IL10, SOD2, CD9 |
| | | | | 219634_AT, 216598_S_AT, | |
| | | | | 226066_AT, 223502_S_AT, 206254_AT, | |
| | | | | 216841_S_AT, 207433_AT, | |
| | | | | 221477_S_AT, 201005_AT | |
| GO:0005887~integral to | 0.001373673 | 2.758590942 | 0.030360284 | 207536_S_AT, 209555_S_AT, | TNFRSF9, CD36, P2RX4, FXYD2, CD40, CD40, |
| plasma membrane | | | | 204088_AT, 207434_S_AT, | IFNGR1, THBD, ICAM1, GPR183, CD9, CD55, |
| | | | | 205153_S_AT, 215346_AT, | ITGB5, GPR34, CD55, STEAP1, ICAM1 |
| | | | | 211676_S_AT, 203887_S_AT, | |
| | | | | 202637_S_AT, 205419_AT, | |
| | | | | 201005_AT, 201926_S_AT, | |
| | | | | 201125_S_AT, 244434_AT, | |
| | | | | 1555950_A_AT, 205542_AT, 202638_S_AT | |
| GO:0050896~response to | 0.0016107 | 1.733869102 | 0.030387009 | 209933_S_AT, 209555_S_AT, | CD300A, CD36, MALT1, P2RX4, CD40, NFKBIA, |
| stimulus | | | | 210017_AT, 204088_AT, | PLAUR, CD40, GHRL, IL7R, CCL2, IL10, NOD2, |
| | | | | 205153_S_AT, 201502_S_AT, 214866_AT, | SOD2, GPR183, CD55, KYNU, CD55, SOD2, |
| | | | | 215346_AT, 223862_AT, | CRYBB2, ADAM9, FABP4, CD14, TNFAIP6, |
| | | | | 226218_AT, 216598_S_AT, 207433_AT, | IFNGR1, TNFSF13B, PTX3, SOD2, THBD, IFIH1, |
| | | | | 220066_AT, 221477_S_AT, | ICAM1, CCL5, KYNU, CD9, TREM1, ICAM1 |
| | | | | 205419_AT, 201926_S_AT, | |
| | | | | 217388_S_AT, 1555950_A_AT, | |
| | | | | 215223_S_AT, 206777_S_AT, 202381_AT, | |
| | | | | 203980_AT, 201743_AT, | |
| | | | | 206026_S_AT, 211676_S_AT, | |
| | | | | 223502_S_AT, 206157_AT, | |
| | | | | 216841_S_AT, 203887_S_AT, | |
| | | | | 219209_AT, 202637_S_AT, | |
| | | | | 1555759_A_AT, 210663_S_AT, | |
| | | | | 201005_AT, 219434_AT, 202638_S_AT | |
| GO:0002700~regulation of | 0.001768391 | 16.2094532 | 0.032085435 | 210017_AT, 207433_AT, 205153_S_AT, | MALT1, IL10, CD40, CD40, NOD2 |
| production of molecular | | | | 215346_AT, 220066_AT | |
| mediator of immune | | | | | |
| GO:0042113~B cell | 0.001821278 | 9.407271945 | 0.032628793 | 210017_AT, 207433_AT, 205153_S_AT, | MALT1, IL10, CD40, CD40, GPR183, IL7R |
| activation | | | | 215346_AT, 205419_AT, 226218_AT | |
| GO:0007165~signal | 0.001988349 | 1.864144476 | 0.034728937 | 209555_S_AT, 210017_AT, 228708_AT, | CD36, MALT1, RAB27B, P2RX4, PLAUR, NFKBIA, |
| transduction | | | | 204088_AT, 214866_AT, | GHRL, IL7R, CCL2, IL10, FGL2, NOD2, GPR183, |
| | | | | 201502_S_AT, 223862_AT, 226218_AT, | ARHGAP18, LPAR6, ADAM9, ARHGAP18, |
| | | | | 216598_S_AT, 207433_AT, | TNFAIP6, MTSS1, IFNGR1, TNFSF13B, EGF, LPXN, |
| | | | | 227265_AT, 220066_AT, 205419_AT, | CCL5, GPR34, TREM1 |
| | | | | 225166_AT, 218589_AT, | |
| | | | | 202381_AT, 225171_AT, | |
| | | | | 206026_S_AT, 203037_S_AT, | |
| | | | | 211676_S_AT, 223502_S_AT, | |
| | | | | 206254_AT, 216250_S_AT, | |
| | | | | 1555759_A_AT, 244434_AT, 219434_AT | |
| GO:0048534~hemopoietic | 0.001967993 | 4.436271401 | 0.034791097 | 215223_S_AT, 216841_S_AT, | SOD2, SOD2, MALT1, IL10, MMP9, MLF1, SOD2, |
| or lymphoid organ | | | | 210017_AT, 207433_AT, | GPR183, VPS33A, IL7R |
| development | | | | 203936_S_AT, 204784_S_AT, | |
| | | | | 221477_S_AT, 205419_AT, | |
| | | | | 204590_X_AT, 226218_AT | |
| GO:0005102~receptor | 0.000373426 | 3.379393637 | 0.037380038 | 202381_AT, 204088_AT, 211725_S_AT, | ADAM9, P2RX4, BID, MTSS1, GHRL, CCL2, |
| binding | | | | 203037_S_AT, 223862_AT, | TNFSF13B, EGF, BID, ADAMDEC1, ICAM1, IL10, |
| | | | | 216598_S_AT, 223502_S_AT, | FGL2, CCL5, BID, ICAM1 |
| | | | | 206254_AT, 227143_S_AT, | |
| | | | | 206134_AT, 202637_S_AT, | |
| | | | | 207433_AT, 227265_AT, | |
| | | | | 1555759_A_AT, 204493_AT, 202638_S_AT | |
| GO:0050794~regulation of | 0.002340559 | 1.349482027 | 0.040285453 | 219358_S_AT, 213891_S_AT, | ADAP2, TCF4, CD36, MALT1, P2RX4, NFE2L3, |
| cellular process | | | | 209555_S_AT, 210017_AT, | NFKBIA, DAB2, AGPAT9, MMP9, NOD2, SOD2, |
| | | | | 204088_AT, 236471_AT, 201502_S_AT, | BIRC3, ARHGAP18, SOD2, LPAR6, ADAM9, |
| | | | | 201278_AT, 224480_S_AT, | TNFRSF9, ARHGAP18, BID, NFE2L3, CHST11, |
| | | | | 203936_S_AT, 220066_AT, | MITF, PTX3, TNFSF13B, EGF, MXD1, SOD2, |
| | | | | 221477_S_AT, 210538_S_AT, | TNFAIP3, CD9, BID, GLRX3, NFKBIE, RAB27B, |
| | | | | 225166_AT, 215223_S_AT, 218589_AT, | CD40, PLAUR, PRDM1, CD40, GHRL, IL7R, TCF4, |
| | | | | 202381_AT, 207536_S_AT, | CCL2, MAFB, HNRPLL, BID, IL10, FGL2, GPR183, |
| | | | | 225171_AT, 211725_S_AT, 204702_S_AT, | FABP4, SRA1, TNFAIP6, MTSS1, IFNGR1, CYTIP, |
| | | | | 219634_AT, 226066_AT, | LPXN, ADAMDEC1, IFIH1, ICAM1, CCL5, GPR34, |
| | | | | 206157_AT, 223502_S_AT, 206254_AT, | TREM1, ICAM1 |
| | | | | 226275_AT, 216841_S_AT, | |
| | | | | 202644_S_AT, 201005_AT, 204493_AT, | |
| | | | | 209080_X_AT, 203927_AT, | |
| | | | | 228708_AT, 205153_S_AT, 214866_AT, | |
| | | | | 228964_AT, 215346_AT, | |
| | | | | 223862_AT, 226218_AT, 212387_AT, | |
| | | | | 216598_S_AT, 218559_S_AT, | |
| | | | | 225386_S_AT, 227143_S_AT, 207433_AT, | |
| | | | | 227265_AT, 205419_AT, | |
| | | | | 203980_AT, 224130_S_AT, | |
| | | | | 206026_S_AT, 203037_S_AT, | |
| | | | | 211676_S_AT, 209606_AT, 216250_S_AT, | |
| | | | | 206134_AT, 219209_AT, | |
| | | | | 202637_S_AT, 1555753_A_AT, | |
| | | | | 244434_AT, 219434_AT, 202638_S_AT | |
| GO:0046651~lymphocyte | 0.002435526 | 14.53261321 | 0.041404807 | 210017_AT, 207433_AT, 205153_S_AT, | MALT1, IL10, CD40, CD40, IL7R |
| proliferation | | | | 215346_AT, 226218_AT | |
| GO:0070661~leukocyte | 0.002687978 | 14.04819277 | 0.045084782 | 210017_AT, 207433_AT, 205153_S_AT, | MALT1, IL10, CD40, CD40, IL7R |
| proliferation | | | | 215346_AT, 226218_AT | |
| GO:0032943~mononuclear | 0.002687978 | 14.04819277 | 0.045084782 | 210017_AT, 207433_AT, 205153_S_AT, | MALT1, IL10, CD40, CD40, IL7R |
| cell proliferation | | | | 215346_AT, 226218_AT | |
| GO:0002520~immune | 0.002858408 | 4.152175203 | 0.047340765 | 215223_S_AT, 216841_S_AT, | SOD2, SOD2, MALT1, IL10, MMP9, MLF1, SOD2, |
| system development | | | | 210017_AT, 207433_AT, | GPR183, VPS33A, IL7R |
| | | | | 203936_S_AT, 204784_S_AT, | |
| | | | | 221477_S_AT, 205419_AT, | |
| | | | | 204590_X_AT, 226218_AT | |
| GO:0042994~cytoplasmic | 0.002996182 | 35.12048193 | 0.049019173 | 203927_AT, 207433_AT, 201502_S_AT | NFKBIE, IL10, NFKBIA |
| sequestering of | | | | | |
| transcription factor | | | | | |
| GO:0042100~B cell | 0.002996182 | 35.12048193 | 0.049019173 | 207433_AT, 205153_S_AT, | IL10, CD40, CD40, IL7R |
| proliferation | | | | 215346_AT, 226218_AT | |

Pathways significantly enriched among genes upregulated by Probioglat relative to GA (mannitol-corrected comparison) at 6 hours, earlier run including Kegg and GO BP only:

| | | | Fold | | | | |
|---|---|---|---|---|---|---|---|
| Category | Term | Pathway | Enrichment | PValue | Benjamini | Probesets | Genes |
| KEGG_{—} | hsa04060 | Cytokine-cytokine receptor | 4.35698707 | 1.27E−04 | 0.009767343 | 204912_AT, 204533_AT, 207536_S_AT, | IL10RA, CXCL10, TNFRSF9, IL10, CD40, CD40, |
| PATHWAY | | interaction | | | | 207433_AT, 205153_S_AT, | CCL5, IFNGR1, IL7R, CCL22, TNFSF13B, EGF |
| | | | | | | 215346_AT, 1555759_A_AT, | |
| | | | | | | 211676_S_AT, 226218_AT, | |
| | | | | | | 216598_S_AT, 223502_S_AT, 206254_AT | |
| KEGG_{—} | hsa04621 | NOD-like receptor signaling | 10.04279131 | 2.62E−04 | 0.010033132 | 202644_S_AT, 201502_S_AT, | TNFAIP3, NFKBIA, CCL5, NOD2, CCL2, BIRC3 |
| PATHWAY | | pathway | | | | 1555759_A_AT, 220066_AT, | |
| | | | | | | 216598_S_AT, 210538_S_AT | |
| GO_BP | GO:0002237 | response to molecule of | 13.53138285 | 1.85E−06 | 0.002747953 | 203887_S_AT, 210017_AT, 201743_AT, | THBD, MALT1, CD14, IL10, NFKBIA, CCL5, NOD2, |
| | | bacterial origin | | | | 207433_AT, 201502_S_AT, | CCL2 |
| | | | | | | 1555759_A_AT, 220066_AT, 216598_S_AT | |
| GO_BP | GO:0006955 | immune response | 3.794670407 | 3.41E−06 | 0.002530005 | 209933_S_AT, 210017_AT, 201743_AT, | CD300A, MALT1, CD14, EBI3, IL7R, CCL2, |
| | | | | | | 219424_AT, 226218_AT, | TNFSF13B, PTX3, CXCL10, IFIH1, ICAM1, IL10, |
| | | | | | | 216598_S_AT, 223502_S_AT, 206157_AT, | CCL5, KYNU, NOD2, GPR183, CD55, KYNU, |
| | | | | | | 204533_AT, 219209_AT, | TREM1, CD55, ICAM1 |
| | | | | | | 202637_S_AT, 207433_AT, | |
| | | | | | | 1555759_A_AT, 210663_S_AT, | |
| | | | | | | 220066_AT, 205419_AT, 201926_S_AT, | |
| | | | | | | 217388_S_AT, 219434_AT, | |
| | | | | | | 1555950_A_AT, 202638_S_AT | |
| GO_BP | GO:0042981 | regulation of apoptosis | 3.437543465 | 6.44E−06 | 0.003180733 | 215223_S_AT, 202381_AT, 207536_S_AT, | SOD2, ADAM9, TNFRSF9, MALT1, P2RX4, BID, |
| | | | | | | 210017_AT, 204088_AT, | NFKBIA, GHRL, CHST11, CCL2, MITF, TNFSF13B, |
| | | | | | | 211725_S_AT, 201502_S_AT, | BID, BTG1, SOD2, TNFAIP3, IFIH1, IL10, MMP9, |
| | | | | | | 223862_AT, 219634_AT, | SOD2, NOD2, BID, BIRC3 |
| | | | | | | 216598_S_AT, 226066_AT, | |
| | | | | | | 223502_S_AT, 227143_S_AT, | |
| | | | | | | 240347_AT, 216841_S_AT, 202644_S_AT, | |
| | | | | | | 219209_AT, 207433_AT, | |
| | | | | | | 203936_S_AT, 221477_S_AT, | |
| | | | | | | 220066_AT, 204493_AT, 210538_S_AT | |
| GO_BP | GO:0043067 | regulation of programmed | 3.403676042 | 7.38E−06 | 0.002735716 | 215223_S_AT, 202381_AT, 207536_S_AT, | SOD2, ADAM9, TNFRSF9, MALT1, P2RX4, BID, |
| | | cell death | | | | 210017_AT, 204088_AT, | NFKBIA, GHRL, CHST11, CCL2, MITF, TNFSF13B, |
| | | | | | | 211725_S_AT, 201502_S_AT, | BID, BTG1, SOD2, TNFAIP3, IFIH1, IL10, MMP9, |
| | | | | | | 223862_AT, 219634_AT, | SOD2, NOD2, BID, BIRC3 |
| | | | | | | 216598_S_AT, 226066_AT, | |
| | | | | | | 223502_S_AT, 227143_S_AT, | |
| | | | | | | 240347_AT, 216841_S_AT, 202644_S_AT, | |
| | | | | | | 219209_AT, 207433_AT, | |
| | | | | | | 203936_S_AT, 221477_S_AT, | |
| | | | | | | 220066_AT, 204493_AT, 210538_S_AT | |
| GO_BP | GO:0010941 | regulation of cell death | 3.391147173 | 7.77E−06 | 0.00230315 | 215223_S_AT, 202381_AT, 207536_S_AT, | SOD2, ADAM9, TNFRSF9, MALT1, P2RX4, BID, |
| | | | | | | 210017_AT, 204088_AT, | NFKBIA, GHRL, CHST11, CCL2, MITF, TNFSF13B, |
| | | | | | | 211725_S_AT, 201502_S_AT, | BID, BTG1, SOD2, TNFAIP3, IFIH1, IL10, MMP9, |
| | | | | | | 223862_AT, 219634_AT, | SOD2, NOD2, BID, BIRC3 |
| | | | | | | 216598_S_AT, 226066_AT, | |
| | | | | | | 223502_S_AT, 227143_S_AT, | |
| | | | | | | 240347_AT, 216841_S_AT, 202644_S_AT, | |
| | | | | | | 219209_AT, 207433_AT, | |
| | | | | | | 203936_S_AT, 221477_S_AT, | |
| | | | | | | 220066_AT, 204493_AT, 210538_S_AT | |
| GO_BP | GO:0009611 | response to wounding | 4.116859404 | 1.28E−05 | 0.003159383 | 215223_S_AT, 209555_S_AT, | SOD2, CD36, CD14, CD40, CD40, TNFAIP6, |
| | | | | | | 201743_AT, 205153_S_AT, | PLAUR, CCL2, PTX3, CXCL10, SOD2, THBD, IL10, |
| | | | | | | 215346_AT, 206026_S_AT, 214866_AT, | MGLL, CCL5, SOD2, CD9, CD55, CD55 |
| | | | | | | 216598_S_AT, 206157_AT, | |
| | | | | | | 204533_AT, 216841_S_AT, 203887_S_AT, | |
| | | | | | | 207433_AT, 239914_AT, | |
| | | | | | | 1555759_A_AT, 221477_S_AT, | |
| | | | | | | 201005_AT, 201926_S_AT, 1555950_A_AT | |
| GO_BP | GO:0032496 | response to | 13.22385142 | 1.35E−05 | 0.0028572 | 203887_S_AT, 201743_AT, | THBD, CD14, IL10, NFKBIA, CCL5, NOD2, CCL2 |
| | | lipopolysaccharide | | | | 207433_AT, 201502_S_AT, | |
| | | | | | | 1555759_A_AT, 220066_AT, 216598_S_AT | |
| GO_BP | GO:0045428 | regulation of nitric oxide | 26.93747511 | 3.12E−05 | 0.005768335 | 215223_S_AT, 216841_S_AT, | SOD2, SOD2, ICAM1, IL10, P2RX4, SOD2, ICAM1, |
| | | biosynthetic process | | | | 202637_S_AT, 207433_AT, | PTX3 |
| | | | | | | 204088_AT, 221477_S_AT, | |
| | | | | | | 202638_S_AT, 206157_AT | |
| GO_BP | GO:0002684 | positive regulation of | 6.1118641 | 3.39E−05 | 0.005574201 | 210017_AT, 205153_S_AT, 215346_AT, | MALT1, CD40, CD40, EBI3, NFKBIA, IL7R, |
| | | immune system process | | | | 219424_AT, 201502_S_AT, | TNFSF13B, CHRNB2, ICAM1, NOD2, CD55, CD55, |
| | | | | | | 226218_AT, 223502_S_AT, 241389_AT, | ICAM1 |
| | | | | | | 202637_S_AT, 220066_AT, | |
| | | | | | | 201926_S_AT, 1555950_A_AT, 202638_S_AT | |
| GO_BP | GO:0002822 | regulation of adaptive | 15.8686217 | 3.47E−05 | 0.005133166 | 210017_AT, 207433_AT, 205153_S_AT, | MALT1, IL10, CD40, CD40, NOD2, IL7R, |
| | | immune response based on | | | | 215346_AT, 220066_AT, | TNFSF13B |
| | | somatic recombination of | | | | 226218_AT, 223502_S_AT | |
| | | immune receptors built | | | | | |
| | | from immunoglobulin | | | | | |
| | | superfamily domains | | | | | |
| GO_BP | GO:0002819 | regulation of adaptive | 15.58525346 | 3.79E−05 | 0.005096778 | 210017_AT, 207433_AT, 205153_S_AT, | MALT1, IL10, CD40, CD40, NOD2, IL7R, |
| | | immune response | | | | 215346_AT, 220066_AT, | TNFSF13B |
| | | | | | | 226218_AT, 223502_S_AT | |
| GO_BP | GO:0060341 | regulation of cellular | 5.865417967 | 4.67E−05 | 0.005763766 | 203927_AT, 202381_AT, 241389_AT, | NFKBIE, ADAM9, CHRNB2, IL10, RAB27B, CD40, |
| | | localization | | | | 207433_AT, 228708_AT, | NFKBIA, CD40, NOD2, GHRL, EGF |
| | | | | | | 205153_S_AT, 201502_S_AT, 215346_AT, | |
| | | | | | | 220066_AT, 223862_AT, 206254_AT | |
| GO_BP | GO:0050878 | regulation of body fluid | 8.253183863 | 4.82E−05 | 0.005486427 | 206835_AT, 213503_X_AT, | STATH, ANXA2P1, CD36, THBD, ANXA2P2, |
| | | levels | | | | 209555_S_AT, 203887_S_AT, | ANXA2P1, CD40, PLAUR, ANXA2P1, CD40, CD9 |
| | | | | | | 208816_X_AT, 210427_X_AT, | |
| | | | | | | 205153_S_AT, 214866_AT, | |
| | | | | | | 201590_X_AT, 215346_AT, 2011305_AT | |
| GO_BP | GO:0006952 | defense response | 3.547862575 | 6.58E−05 | 0.006947618 | 210017_AT, 201743_AT, 205153_S_AT, | MALT1, CD14, CD40, CD40, TNFAIP6, CCL2, |
| | | | | | | 215346_AT, 206026_S_AT, | PTX3, CXCL10, TPSAB1, IFIH1, IL10, MGLL, CCL5, |
| | | | | | | 216598_S_AT, 206157_AT, 204533_AT, | NOD2, KYNU, CD55, KYNU, CD55 |
| | | | | | | 207741_X_AT, 219209_AT, | |
| | | | | | | 207433_AT, 239914_AT, | |
| | | | | | | 1555759_A_AT, 220066_AT, | |
| | | | | | | 210663_S_AT, 201926_S_AT, | |
| | | | | | | 217388_S_AT, 1555950_A_AT | |
| GO_BP | GO:0051046 | regulation of secretion | 6.480396487 | 6.85E−05 | 0.006751625 | 202381_AT, 241389_AT, 207433_AT, | ADAM9, CHRNB2, IL10, RAB27B, P2RX4, CD40, |
| | | | | | | 228708_AT, 204088_AT, | CD40, NOD2, GHRL, EGF |
| | | | | | | 205153_S_AT, 215346_AT, 220066_AT, | |
| | | | | | | 223862_AT, 206254_AT | |
| GO_BP | GO:0042127 | regulation of cell | 3.142134962 | 7.26E−05 | 0.006714161 | 215223_S_AT, 207536_S_AT, | SOD2, TNFRSF9, FABP4, CD40, NFKBIA, EBI3, |
| | | proliferation | | | | 203980_AT, 205153_S_AT, | CD40, GHRL, CHST11, CCL2, MITF, TNFSF13B, |
| | | | | | | 201502_S_AT, 219424_AT, 215346_AT, | EGF, BTG1, CXCL10, SOD2, CHRNB2, IL10, SOD2, |
| | | | | | | 223862_AT, 219634_AT, | CD9 |
| | | | | | | 216598_S_AT, 226066_AT, 223502_S_AT, | |
| | | | | | | 206254_AT, 240347_AT, | |
| | | | | | | 204533_AT, 216841_S_AT, 241389_AT, | |
| | | | | | | 207433_AT, 221477_S_AT, 201005_AT | |
| GO_BP | GO:0006916 | anti-apoptosis | 6.355151895 | 7.86E−05 | 0.006835343 | 215223_S_AT, 216841_S_AT, | SOD2, SOD2, TNFAIP3, MALT1, IL10, NFKBIA, |
| | | | | | | 202644_S_AT, 210017_AT, | SOD2, GHRL, CCL2, TNFSF13B, BIRC3 |
| | | | | | | 207433_AT, 201502_S_AT, | |
| | | | | | | 221477_S_AT, 223862_AT, | |
| | | | | | | 216598_S_AT, 223502_S_AT, 210538_S_AT | |
| GO_BP | GO:0051223 | regulation of protein | 8.931899642 | 1.24E−04 | 0.010183221 | 203927_AT, 202381_AT, 207433_AT, | NFKBIE, ADAM9, IL10, CD40, NFKBIA, CD40, |
| | | transport | | | | 205153_S_AT, 201502_S_AT, | NOD2, EGF |
| | | | | | | 215346_AT, 220066_AT, 206254_AT | |
| GO_BP | GO:0043066 | negative regulation of | 4.52001701 | 1.42E−04 | 0.011048668 | 215223_S_AT, 210017_AT, 201502_S_AT, | SOD2, MALT1, NFKBIA, GHRL, CHST11, CCL2, |
| | | apoptosis | | | | 223862_AT, 219634_AT, | MITF, TNFSF13B, SOD2, TNFAIP3, IL10, SOD2, |
| | | | | | | 216598_S_AT, 226066_AT, | BIRC3 |
| | | | | | | 223502_S_AT, 216841_S_AT, | |
| | | | | | | 202644_S_AT, 207433_AT, | |
| | | | | | | 221477_S_AT, 210538_S_AT | |
| GO_BP | GO:0001775 | cell activation | 5.068375108 | 1.43E−04 | 0.010557063 | 202381_AT, 210017_AT, 241389_AT, | ADAM9, MALT1, CHRNB2, ICAM1, IL10, P2RX4, |
| | | | | | | 202637_S_AT, 207433_AT, | CD40, CD40, GPR183, CD9, IL7R, ICAM1 |
| | | | | | | 204088_AT, 205153_S_AT, 215346_AT, | |
| | | | | | | 205419_AT, 201005_AT, | |
| | | | | | | 226218_AT, 202638_S_AT | |
| GO_BP | GO:0043069 | negative regulation of | 4.457064127 | 1.59E−04 | 0.011205923 | 215223_S_AT, 210017_AT, 201502_S_AT, | SOD2, MALT1, NFKBIA, GHRL, CHST11, CCL2, |
| | | programmed cell death | | | | 223862_AT, 219634_AT, | MITF, TNFSF13B, SOD2, TNFAIP3, IL10, SOD2, |
| | | | | | | 216598_S_AT, 226066_AT, | BIRC3 |
| | | | | | | 223502_S_AT, 216841_S_AT, | |
| | | | | | | 202644_S_AT, 207433_AT, | |
| | | | | | | 221477_S_AT, 210538_S_AT | |
| GO_BP | GO:0060548 | negative regulation of cell | 4.444683393 | 1.63E−04 | 0.010942714 | 215223_S_AT, 210017_AT, 201502_S_AT, | SOD2, MALT1, NFKBIA, GHRL, CHST11, CCL2, |
| | | death | | | | 223862_AT, 219634_AT, | MITF, TNFSF13B, SOD2, TNFAIP3, IL10, SOD2, |
| | | | | | | 216598_S_AT, 226066_AT, | BIRC3 |
| | | | | | | 223502_S_AT, 216841_S_AT, | |
| | | | | | | 202644_S_AT, 207433_AT, | |
| | | | | | | 221477_S_AT, 210538_S_AT | |
| GO_BP | GO:0042113 | B cell activation | 11.48387097 | 1.65E−04 | 0.010566072 | 210017_AT, 241389_AT, 207433_AT, | MALT1, CHRNB2, IL10, CD40, CD40, GPR183, |
| | | | | | | 205153_S_AT, 215346_AT, | IL7R |
| | | | | | | 205419_AT, 226218_AT | |
| GO_BP | GO:0070201 | regulation of establishment | 8.415178175 | 1.72E−04 | 0.01059381 | 203927_AT, 202381_AT, 207433_AT, | NFKBIE, ADAM9, IL10, CD40, NFKBIA, CD40, |
| | | of protein localization | | | | 205153_S_AT, 201502_S_AT, | NOD2, EGF |
| | | | | | | 215346_AT, 220066_AT, 206254_AT | |
| GO_BP | GO:0046903 | secretion | 4.84874552 | 1.99E−04 | 0.011766402 | 201278_AT, 204533_AT, 206835_AT, | DAB2, CXCL10, STATH, ANXA2P1, CHRNB2, |
| | | | | | | 213503_X_AT, 241389_AT, | ANXA2P2, ANXA2P1, ANXA2P1, CCL5, VPS33A, |
| | | | | | | 208816_X_AT, 210427_X_AT, | GHRL, TNFSF13B |
| | | | | | | 201590_X_AT, 1555759_A_AT, | |
| | | | | | | 204590_X_AT, 223862_AT, 223502_S_AT | |
| GO_BP | GO:0009617 | response to bacterium | 6.029528107 | 3.40E−04 | 0.019214892 | 203887_S_AT, 210017_AT, 201743_AT, | THBD, MALT1, CD14, IL10, NFKBIA, CCL5, NOD2, |
| | | | | | | 207433_AT, 201502_S_AT, | CCL2 |
| | | | | | | 1555759_A_AT, 220066_AT, 216598_S_AT | |
| GO_BP | GO:0010033 | response to organic | 3.026262807 | 3.46E−04 | 0.018159624 | 202381_AT, 210017_AT, 201743_AT, | ADAM9, MALT1, CD14, FABP4, P2RX4, NFKBIA, |
| | | substance | | | | 203980_AT, 204088_AT, | GHRL, HMGB2, CCL2, THBD, CHRNB2, IL10, |
| | | | | | | 201502_S_AT, 223862_AT, | CCL5, NOD2, KYNU, KYNU |
| | | | | | | 243368_AT, 216598_S_AT, | |
| | | | | | | 203887_S_AT, 241389_AT, | |
| | | | | | | 207433_AT, 1555759_A_AT, | |
| | | | | | | 220066_AT, 210663_S_AT, 217388_S_AT | |
| GO_BP | GO:0032880 | regulation of protein | 7.378525791 | 3.51E−04 | 0.018439313 | 203927_AT, 202381_AT, 207433_AT, | NFKBIE, ADAM9, IL10, CD40, NFKBIA, CD40, |
| | | localization | | | | 205153_S_AT, 201502_S_AT, | NOD2, EGF |
| | | | | | | 215346_AT, 220066_AT, 2006254_AT | |
| GO_BP | GO:0006954 | inflammatory response | 4.475765095 | 3.60E−04 | 0.018262369 | 204533_AT, 201743_AT, 207433_AT, | CXCL10, CD14, IL10, MGLL, CD40, CD40, |
| | | | | | | 239914_AT, 205153_S_AT, | TNFAIP6, CCL5, CD55, CCL2, CD55, PTX3 |
| | | | | | | 215346_AT, 206026_S_AT, | |
| | | | | | | 1555759_A_AT, 201926_S_AT, | |
| | | | | | | 216598_S_AT, 1555950_A_AT, 206157_AT | |
| GO_BP | GO:0045429 | positive regulation of nitric | 27.70711726 | 3.70E−04 | 0.018161517 | 215223_S_AT, 216841_S_AT, | SOD2, SOD2, ICAM1, P2RX4, SOD2, ICAM1, |
| | | oxide biosynthetic process | | | | 202637_S_AT, 204088_AT, | PTX3 |
| | | | | | | 221477_S_AT, 202638_S_AT, 206157_AT | |
| GO_BP | GO:0002706 | regulation of lymphocyte | 13.46873755 | 4.89E−04 | 0.023119461 | 210017_AT, 207433_AT, 205153_S_AT, | MALT1, IL10, CD40, CD40, NOD2, IL7R |
| | | mediated immunity | | | | 215346_AT, 220066_AT, 226218_AT | |
| GO_BP | G0:0051249 | regulation of lymphocyte | 6.879976751 | 5.10E−04 | 0.023373393 | 210017_AT, 241389_AT, 207433_AT, | MALT1, CHRNB2, IL10, CD40, EBI3, CD40, IL7R, |
| | | activation | | | | 205153_S_AT, 219424_AT, | TNFSF13B |
| | | | | | | 215346_AT, 226218_AT, 223502_S_AT | |
| GO_BP | GO:0051251 | positive regulation of | 8.997672098 | 5.13E−04 | 0.022828997 | 210017_AT, 241389_AT, 205153_S_AT, | MALT1, CHRNB2, CD40, EBI3, CD40, IL7R, |
| | | lymphocyte activation | | | | 219424_AT, 215346_AT, | TNFSF13B |
| | | | | | | 226218_AT, 223502_S_AT | |
| GO_BP | GO:0002697 | regulation of immune | 8.641328649 | 6.18E−04 | 0.026609275 | 210017_AT, 202637_S_AT, 207433_AT, | MALT1, ICAM1, IL10, CD40, CD40, NOD2, IL7R, |
| | | effector process | | | | 205153_S_AT, 215346_AT, | ICAM1 |
| | | | | | | 220066_AT, 226218_AT, 202638_S_AT | |
| GO_BP | GO:0050708 | regulation of protein | 12.5398591 | 6.42E−04 | 0.026859383 | 202381_AT, 207433_AT, 205153_S_AT, | ADAM9, IL10, CD40, CD40, NOD2, EGF |
| | | secretion | | | | 215346_AT, 220066_AT, 206254_AT | |
| GO_BP | GO:0032101 | regulation of response to | 6.404003517 | 7.44E−04 | 0.030210271 | 213503_X_AT, 203980_AT, | ANXA2P1, FABP4, ANXA2P2, IL10, ANXA2P1, |
| | | external stimulus | | | | 208816_X_AT, 207433_AT, | ANXA2P1, CCL5, GHRL, NTSE |
| | | | | | | 210427_X_AT, 201590_X_AT, | |
| | | | | | | 1555759_A_AT, 223862_AT, 203939_AT | |
| GO_BP | GO:0002696 | positive regulation of | 8.233718807 | 7.70E−04 | 0.030405582 | 210017_AT, 241389_AT, 205153_S_AT, | MALT1, CHRNB2, CD40, EBI3, CD40, IL7R, |
| | | leukocyte activation | | | | 219424_AT, 215346_AT, | TNFSF13B |
| | | | | | | 226218_AT, 223502_S_AT | |
| GO_BP | GO:0002703 | regulation of leukocyte | 11.922314472 | 7.77E−04 | 0.029915725 | 210017_AT, 207433_AT, 205153_S_AT, | MALT1, IL10, CD40, CD40, NOD2, IL7R |
| | | mediated immunity | | | | 215346_AT, 220066_AT, 226218_AT | |
| GO_BP | GO:0051050 | positive regulation of | 5.218380828 | 8.05E−04 | 0.030169382 | 202381_AT, 241389_AT, 207433_AT, | ADAM9, CHRNB2, IL10, P2RX4, NFKBIA, NOD2, |
| | | transport | | | | 204088_AT, 201502_S_AT, | GHRL, PTX3 |
| | | | | | | 220066_AT, 223862_AT, 206157_AT | |
| GO_BP | GO:0007155 | cell adhesion | 2.909247312 | 8.70E−04 | 0.031759691 | 209933_S_AT, 213428_S_AT, | CD300A, COL6A1, ADAM9, CD36, ATP2C1, |
| | | | | | | 202381_AT, 209555_S_AT, | TNFAIP6, MTSS1, ICAM2, CCL2, LPXN, ICAM1, |
| | | | | | | 237278_X_AT, 206026_S_AT, | CCL5, CD9, ITGB5, ICAM1 |
| | | | | | | 203037_S_AT, 213620_S_AT, | |
| | | | | | | 216598_S_AT, 216250_S_AT, | |
| | | | | | | 202637_S_AT, 1555759_A_AT, | |
| | | | | | | 201005_AT, 201125_S_AT, 202638_S_AT | |
| GO_BP | GO:0051047 | positive regulation of | 8.007102693 | 8.73E−04 | 0.031121915 | 202381_AT, 241389_AT, 207433_AT, | ADAM9, CHRNB2, IL10, P2RX4, NOD2, GHRL |
| | | secretion | | | | 204088_AT, 220066_AT, 223862_AT | |
| GO_BP | GO:0051241 | negative regulation of | 6.208759507 | 8.74E−04 | 0.030436041 | 206835_AT, 213503_X_AT, 208816_X_AT, | STATH, ANXA2P1, ANXA2P2, IL10, P2RX4, |
| | | multicellular organismal | | | | 207433_AT, 204088_AT, | ANXA2P1, ANXA2P1, NOD2, GHRL |
| | | process | | | | 210427_X_AT, 201590_X_AT, | |
| | | | | | | 220066_AT, 223862_AT | |
| GO_BP | GO:0022610 | biological adhesion | 2.905097173 | 8.81E−04 | 0.029962986 | 209933_S_AT, 213428_S_AT, | CD300A, COL6A1, ADAM9, CD36, ATP2C1, |
| | | | | | | 202381_AT, 209555_S_AT, | TNFAIP6, MTSS1, ICAM2, CCL2, LPXN, ICAM1, |
| | | | | | | 237278_X_AT, 206026_S_AT, | CCL5, CD9, ITGB5, ICAM1 |
| | | | | | | 203037_S_AT, 213620_S_AT, | |
| | | | | | | 216598_S_AT, 216250_S_AT, | |
| | | | | | | 202637_S_AT, 1555759_A_AT, | |
| | | | | | | 201005_AT, 201125_S_AT, 202638_S_AT | |
| GO_BP | GO:0030888 | regulation of B cell | 20.78033794 | 8.82E−04 | 0.029309007 | 241389_AT, 207433_AT, 205153_S_AT, | CHRNB2, IL10, CD40, CD40, TNFSF13B |
| | | proliferation | | | | 215346_AT, 223502_S_AT | |
| GO_BP | GO:0002694 | regulation of leukocyte | 6.133955176 | 9.31E−04 | 0.030258714 | 210017_AT, 241389_AT, 207433_AT, | MALT1, CHRNB2, IL10, CD40, EBI3, CD40, IL7R, |
| | | activation | | | | 205153_S_AT, 219424_AT, | TNFSF13B |
| | | | | | | 215346_AT, 226218_AT, 223502_S_AT | |
| GO_BP | GO:0050867 | positive regulation of cell | 7.862830573 | 9.48E−04 | 0.030125327 | 210017_AT, 241389_AT, 205153_S_AT, | MALT1, CHRNB2, CD40, EBI3, CD40, IL7R, |
| | | activation | | | | 219424_AT, 215346_AT, | TNFSF13B |
| | | | | | | 226218_AT, 223502_S_AT | |
| GO_BP | GO:0048584 | positive regulation of | 4.930927647 | 0.001120224 | 0.034771388 | 210017_AT, 203980_AT, | MALT1, FABP4, NFKBIA, CCL5, NOD2, GHRL, |
| | | response to stimulus | | | | 201502_S_AT, 1555759_A_AT, | CD5S, TNFSF13B, CD55 |
| | | | | | | 220066_AT, 223862_AT, | |
| | | | | | | 201926_S_AT, 223502_S_AT, 1555950_A_AT | |
| GO_BP | GO:0030097 | hemopoiesis | 4.930927647 | 0.001120224 | 0.034771388 | 215223_S_AT, 216841_S_AT, | SOD2, SOD2, MALT1, IL10, MMP9, MLf1, SOD2, |
| | | | | | | 210017_AT, 207433_AT, | GPR183, VPS33A, IL7R |
| | | | | | | 203936_S_AT, 204784_S_AT, | |
| | | | | | | 221477_S_AT, 205419_AT, | |
| | | | | | | 204590_X_AT, 226218_AT | |
| GO_BP | GO:0032680 | regulation of tumor | 18.7693375 | 0.00119195 | 0.03620158 | 201743_AT, 207433_AT, | CD14, IL10, NOD2, GHRL |
| | | necrosis factor production | | | | 220066_AT, 223862_AT | |
| GO_BP | GO:0050865 | regulation of cell activation | 5.818494624 | 0.001223425 | 0.036396133 | 210017_AT, 241389_AT, 207433_AT, | MALT1, CHRNB2, IL10, CD40, EBI3, CD40, IL7R, |
| | | | | | | 205153_S_AT, 219424_AT, | TNFSF13B |
| | | | | | | 215346_AT, 226218_AT, 223502_S_AT | |
| GO_BP | GO:0045321 | leukocyte activation | 4.808673243 | 0.00129524 | 0.037737147 | 202381_AT, 210017_AT, 241389_AT, | ADAM9, MALT1, CHRNB2, ICAM1, IL10, CD40, |
| | | | | | | 202637_S_AT, 207433_AT, | CD40, GPR183, IL7R, ICAM1 |
| | | | | | | 205153_S_AT, 215346_AT, 205419_AT, | |
| | | | | | | 226218_AT, 202638_S_AT | |
| GO_BP | GO:0051173 | positive regulation of | 2.9363521 | 0.001352769 | 0.038623899 | 213891_S_AT, 215223_S_AT, | TCF4, SOD2, CD40, P2RX4, SRA1, CD40, NFKBIA, |
| | | nitrogen compound | | | | 205153_S_AT, 204088_AT, | HMGB2, TCF4, MITF, MAFB, PTX3, HNRPLL, |
| | | metabolic process | | | | 224130_S_AT, 215346_AT, 201502_S_AT, | SOD2, ICAM1, IL10, SOD2, ICAM1 |
| | | | | | | 243368_AT, 212387_AT, | |
| | | | | | | 226066_AT, 218559_S_AT, | |
| | | | | | | 206157_AT, 225386_S_AT, | |
| | | | | | | 216841_S_AT, 202637_S_AT, | |
| | | | | | | 207433_AT, 221477_S_AT, 202638_S_AT | |
| GO_BP | GO:0051240 | positive regulation of | 4.769257888 | 0.001358112 | 0.03804226 | 210017_AT, 241389_AT, 201743_AT, | MALT1, CHRNB2, CD14, CD40, CD40, CCL5, |
| | | multicellular organismal | | | | 205153_S_AT, 215346_AT, | NOD2, GHRL, CCL2 |
| | | process | | | | 1555759_A_AT, 220066_AT, | |
| | | | | | | 223862_AT, 216598_S_AT | |
| GO_BP | GO:0001817 | regulation of cytokine | 5.625616349 | 0.001454089 | 0.039925244 | 210017_AT, 201743_AT, 207433_AT, | MALT1, CD14, IL10, CD40, EBI3, CD40, NOD2, |
| | | production | | | | 205153_S_AT, 219424_AT, | GHRL |
| | | | | | | 215346_AT, 220066_AT, 223862_AT | |
| GO_BP | GO:0050727 | regulation of inflammatory | 9.569892473 | 0.001768848 | 0.047488977 | 203980_AT, 207433_AT, 1555759_A_AT, | FABP4, IL10, CCL5, GHRL, NT5E |
| | | response | | | | 223862_AT, 203939_AT | |
| GO_BP | GO:0010647 | positive regulation of cell | 3.979213648 | 0.001776941 | 0.046854541 | 210017_AT, 241389_AT, 204088_AT, | MALT1, CHRNB2, P2RX4, ATP2C1, CD40, CD40, |
| | | communication | | | | 237278_X_AT, 205153_S_AT, | NOD2, GHRL, CCL2, EGF |
| | | | | | | 215346_AT, 220066_AT, 223862_AT, | |
| | | | | | | 216598_S_AT, 206254_AT | |
| GO_BP | GO:0002250 | adaptive immune response | 9.445608155 | 0.001856176 | 0.048042002 | 202637_S_AT, 207433_AT, 219424_AT, | ICAM1, IL10, EBI3, NOD2, CD55, ICAM1, CD55 |
| | | | | | | 220066_AT, 201926_S_AT, | |
| | | | | | | 202638_S_AT, 1555950_A_AT | |
| GO_BP | GO:0002460 | adaptive immune response | 9.445608155 | 0.001856176 | 0.048042002 | 202637_S_AT, 207433_AT, 219424_AT, | ICAM1, IL10, EBI3, NOD2, CD55, ICAM1, CD55 |
| | | based on somatic | | | | 220066_AT, 201926_S_AT, | |
| | | recombination of immune | | | | 202638_S_AT, 1555950_A_AT | |
| | | receptors built from | | | | | |
| | | immunoglobulin | | | | | |
| | | superfamily domains | | | | | |
| GO_BP | GO:0051384 | response to glucocorticoid | 9.324510615 | 0.00194645 | 0.043450316 | 202381_AT, 203980_AT, | ADAM9, FABP4, IL10, CCL5, CCL2 |
| | | stimulus | | | | 207433_AT, 1555759_A_AT, 216598_S_AT | |
| GO_BP | GO:0048534 | hemopoietic or lymphoid | 4.475765095 | 0.001951531 | 0.048752558 | 215223_S_AT, 216841_S_AT, | SOD2, SOD2, MALT1, IL10, MMP9, MLF1, SOD2, |
| | | organ development | | | | 210017_AT, 207433_AT, | GPR183, VPS33A, IL7R |
| | | | | | | 203936_S_AT, 204784_S_AT, | |
| | | | | | | 221477_S_AT, 205419_AT, | |
| | | | | | | 204590_X_AT, 226218_AT | |
| GO_BP | GO:0048545 | response to steroid | 5.303315412 | 0.001961629 | 0.048188649 | 202381_AT, 203980_AT, 207433_AT, | ADAM9, FABP4, IL10, CCL5, GHRL, HMGB2, |
| | | hormone stimulus | | | | 1555759_A_AT, 223862_AT, | CCL2 |
| | | | | | | 243368_AT, 216598_S_AT | |
| GO_BP | GO:0006959 | humoral immune response | 9.206478835 | 0.00203972 | 0.049246639 | 219424_AT, 205419_AT, 201926_S_AT, | EBI3, GPR183, CD55, TREM1, CCL2, CD55 |
| | | | | | | 219434_AT, 216598_S_AT, 1555950_A_AT | |

### Example 4

### Validation of Pro-Inflammatory Markers' Upregulation by Stimulation with Probioglat Compared to Copaxone® (gRT-PCR Analysis)

Key genes identified by differential expression analysis were assayed using qRT-PCR. RNA was utilized from each of 6 biological samples for each treatment (Copaxone and Probioglat) and 15 technical replicates were performed for each sample (a total of 90 observations per transcript per treatment). Since three Copaxone® batches and one Probioglat batch were available, a total of 360 observations from each transcript were evaluated. To evaluate the data, the 2^{-ΔΔct} approximation was utilized with GAPDH as reference transcript and vehicle control (mannitol) as calibrator. A one-sided t-test with unequal variance was used to compare the RNA expression from the two treatments.

To validate the results from the microarrays comparing Probioglat with Copaxone® for key inflammation and MS-related genes, two chemokines (CCL5, FDR p-value<0.02 and CXCL10, FDR p-value<0.0006), two matrix metalloproteinases (MMP1, FDR p-value<0.002 and MMP9, FDR p-value<2.8e-6) and a non-secreted cell surface marker (CD9, FDR p-value<0.002 with FC 1.15) that is a component of myelin and a marker of myelinogenic progenitor cells (Allie et al., Arch. Neurol. 2005) were tested independently by robust qRT-PCR analysis. Three Copaxone® batches and one Probioglat batch were available for use, and a total of 360 observations from each transcript were evaluated. Statistical analysis utilized a one-sided t-test with unequal variance to compare the RNA expression from the two treatments. All the genes tested were significantly differentially expressed between Probioglat and Copaxone as expected based on the microarray analysis (Table 7).

TABLE 7
Differential expression (p value and fold change) of key immunological genes
following Probioglat stimulation compared with Copaxone ®

| | Genes | | | | | | | | | |
|---|---|---|---|---|---|---|---|---|---|---|
| | CCL5 | | CD9 | | CXCL10 | | MMP1 | | MMP9 | |
| Method | FC | p value | FC | p value | FC | p value | FC | p value | FC | p value |
| qPCR | 1.12 | 4.05E−05 | 1.11 | 0.0004 | 2.28 | 0.0029 | 1.25 | 0.0201 | 1.24 | 0.0168 |
| FDR- | 1.09 | 0.02 | 1.15 | 0.002 | 1.46 | 0.0006 | 1.5 | 0.002 | 1.29 | 2.80E−06 |
| adjusted | | | | | | | | | | |
| Microarray | | | | | | | | | | |

FC: fold change; qPCR; quantitative RT-PCR; FDR: For the microarray data, since all probesets on the microarray were tested, p values were adjusted using FDR for testing multiple hypotheses.

Table 7 shows p-values from single-tailed t-test with unequal variance (for qPCR results) and FDR-adjusted p-values from LIMMA comparison of microarray data between human monocytes treated with Copaxone® and Probioglat.

### Discussion

### Examples 3-4

The genes significantly upregulated (FDR adjusted p value<0.05) in Probioglat relative to Copaxone® treatment at 6 hours were found to be enriched significantly (Benjamini corrected p value<0.05) for 106 pathways annotated in the GO (Biological Process, Cellular Component, and Molecular Function) and Kegg databases (The Gene Ontology Consortium. Gene ontology: tool for the unification of biology, Nat. Genet., May 2000; Kanehisa et al, KEGG: Kyoto Encyclopedia of Genes and Genomes, N A R, 2000) (FIG. 10; Table 6). These include immune system process (GO:0002376), response to lipopolysaccharide (LPS) (GO:0032496), and immune response (GO:0006955) pathways (Benjamini corrected p values 1.5e-5, 8.7e-4, and 3.3e-4, respectively). Several of these pathways are relevant to inflammation (e.g., regulation of inflammatory response (GO:0050727) and regulation of tumor necrosis factor production (GO:0032680), Benjamini corrected p values of 0.015 and 0.028, respectively).

Branded GA significantly modulated many validated pathways. At 6 hours, pathways enriched significantly among upregulated genes included broad categories such as immune response and regulation of immune processes, and more specifically cytokine-cytokine receptor interactions. Other significantly enriched pathways included adhesion; extracellular region; plasma membrane; membrane; response to external stimulus; response to stress; response to wounding; defense response; inflammatory response; and immune system process, all pathways with broad relevance to the disease process and/or proposed action of GA. Several of these pathways (e.g., extracellular region; immune system process; defense response; regulation of leukocyte activation) were also seen significantly enriched among genes modulated by GA in monocytes obtained from RRMS patients within the first two months of treatment (Thamilarasan, J Neuroinflammation 2013).

As another example, NOD-like receptor signaling (hsa04621, Benjamini corrected p value 0.027) regulates inflammatory and apoptotic responses. The response to LPS pathway (GO:0050727; FIG. 9) includes the genes CD14, CCL5, THBD, CARD15, NFKBIA, and CCL2, all upregulated in Probioglat treatment versus GA at 6 hours. This pathway was also significantly enriched among probesets upregulated by GA treatment at 6 hours, though with a lower enrichment score (14.8 vs 2.7) and higher p value (0.00087 vs 0.036). The strong enrichment induced by Probioglat relative to GA of this prototypical pro-inflammatory pathway warrants further investigation with respect to safety.

Interestingly, about half of the pathways (58 out of 114) significantly enriched (Benjamini corrected p value<0.05) among genes upregulated by GA treatment versus mannitol control at 6 hours were also significantly enriched among genes upregulated by Probioglat relative to GA treatment. An additional 48 pathways were significantly enriched among genes upregulated by Probioglat relative to GA (and not modulated by GA relative to mannitol control). These include pathways relevant to inflammation, such as response to molecule of bacterial origin (GO:0002237), regulation of tumor necrosis factor production (GO:0032680) and NOD-like receptor signaling pathway (hsa04621), as well as other immune pathways including regulation of lymphocyte mediated immunity (GO:0002706) and B cell proliferation (GO:0042100).

### Example 5

### Integrated Analysis Spanning Multiple Species and Cell Types

Analyses were conducted to elucidate the gene expression changes induced by Copaxone® in the following systems:
1) primed, ex vivo mouse splenocytes;
2) THP-1 human monocyte cell line; and
3) samples from MS patients.

Genes, pathways and immune cell types modulated by Copaxone® were investigated, in order to determine which aspects of Copaxone®'s mechanism were observed across all systems utilized, and which were detectable only in certain systems, but not others.

Genome-wide expression profiles in cells from three different datasets in two different species (human, mouse) were studied. LIMMA was utilized to identify a genome-wide list of differentially expressed genes induced by GA in the primed and ex vivo stimulated mouse splenocytes, as well as in the THP-1 human monocyte cell line. Repeated-measures ANOVA was utilized to find a genome-wide list of genes modulated by GA in treated MS patients. Advanced enrichment algorithms were then applied to elucidate the pathways and cell types modulated by GA.

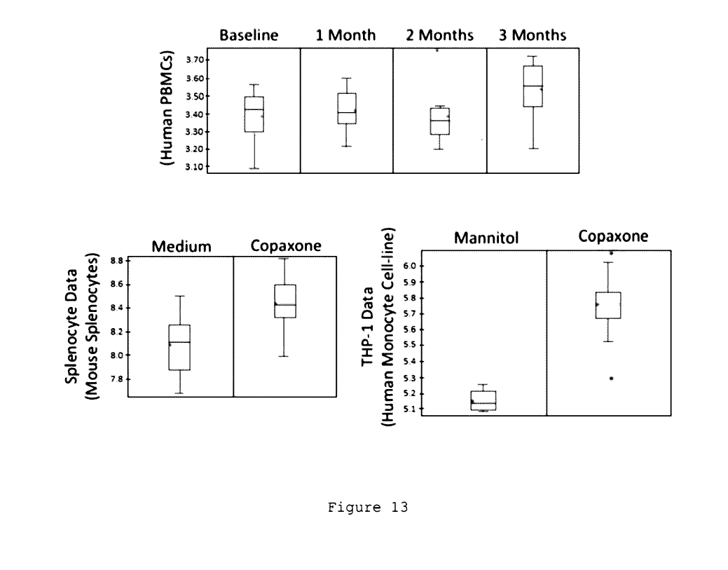

Upregulated expression of the IL-10 gene, a key indicator of the well-studied Th2-shift induced by GA, was consistently demonstrated in all 3 systems (mouse splenocytes, human monocytes and MS patient PBMCs) (FIG. 13). GA induces an anti-inflammatory effect, mediated by secretion of IL-4, IL-10, and other anti-inflammatory cytokines. This effect involves both a shift in T cell populations (from pro-inflammatory Th1 to anti-inflammatory Th2) and a shift from monocyte production of IL12 to anti-inflammatory IL10. For example, in vitro GA treatment increased the proportion of IL10-producing Treg cells in blood from MS patients (Putheti, J Neuroimmunol 2003). Dendritic cells exposed to GA during maturation increased their production of IL10 (Vieira et al, J Immunol 2003), monocytes from mice treated with GA secreted more IL10 than monocytes from untreated mice (Weber et al, Nat Med 2007), and monocytes isolated from MS patients treated with GA were shown to upregulate IL10 relative to untreated patients (Kim et al, J Immunol 2004).

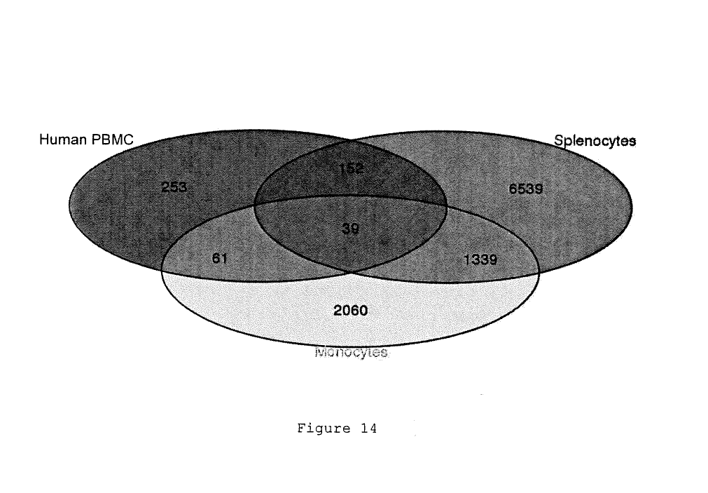

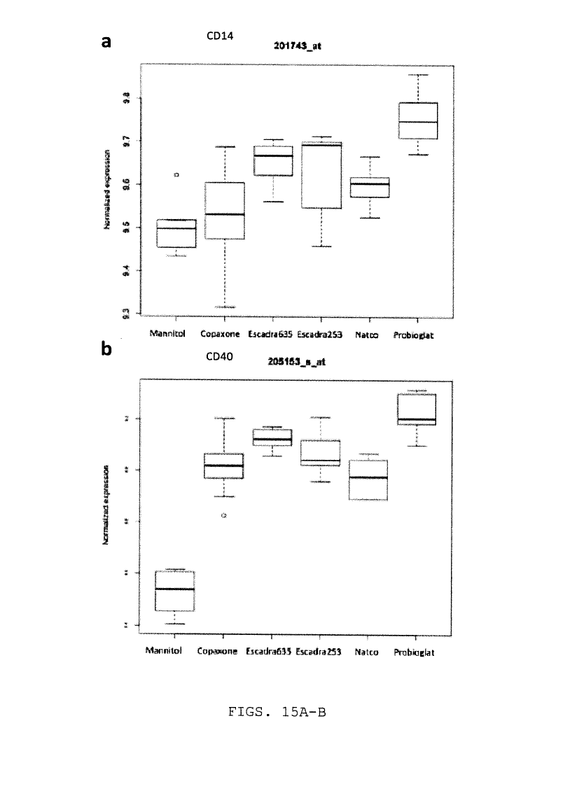

The genes modulated by GA treatment in multiple studies were examined for enrichment in particular immunological cell types. 39 genes were modulated significantly by GA in all three studies (FIG. 14). Table 8 shows the expression level of each gene as modulated by Copaxone® compared to baseline (human PBMC), mannitol (human monocyte) or medium (mouse splenocytes). Although some genes are significantly differentially expressed in the same direction across all compartments and systems, other genes are modulated differently across the different systems. This shows that Copaxone® may induce pathways or cell types differently depending on a given experimental setup and a well-designed genome-wide assay is required to ascertain mechanism and effect of Copaxone®. Starred genes did not meet effect size cutoff of 0.5 employed in human PBMC study for conducting enrichments.

TABLE 8
Genes significantly modulated by Copaxone ® treatment in
all three studies

| | Direction in | Direction in | Direction in |
|---|---|---|---|
| | human | human | mouse |
| | PBMC | monocytes | splenocytes |
| | (Copaxone | (Copaxone | (Copaxone |
| | relative to | relative to | relative to |
| Gene | Baseline) | Mannitol) | Medium) |
| ABCF2 | DOWN | DOWN | UP |
| ABI2 | UP | UP | UP |
| ACP6 | DOWN | DOWN | DOWN |
| AFG3L2 | DOWN | DOWN | UP |
| ALMS1 | DOWN | UP | UP |
| ARPC4 | UP | UP | UP |
| CALM3 | UP | DOWN | DOWN |
| CCDC64 | UP | DOWN | DOWN |
| CD84 | UP | UP | DOWN |
| CDC6 | UP | DOWN | UP |
| CHAF1A | DOWN | DOWN | UP |
| CLU | UP | UP | DOWN |
| COX11 | DOWN | DOWN | UP |
| DLGAP1 | DOWN | UP | DOWN |
| DTX4 | DOWN | UP | DOWN |
| FAM49B | DOWN | UP | UP |
| FHL1 | UP | DOWN | DOWN |
| FNTB | UP | DOWN | DOWN |
| GYPC | UP | DOWN | DOWN |
| HFE | UP | UP | UP |
| IL10* | UP | UP | UP |
| LPHN1 | DOWN | DOWN | DOWN |
| NACA* | DOWN | DOWN | UP |
| CLAH | DOWN | DOWN | UP |
| PATZ1 | UP | DOWN | DOWN |
| PDK1 | UP | DOWN | DOWN |
| POLI | DOWN | DOWN | DOWN |
| REEP5 | DOWN | UP | DOWN |
| RPL5* | DOWN | UP | DOWN |
| RPS6KA2 | DOWN | UP | DOWN |
| SEC31A | DOWN | DOWN | DOWN |
| SETBP1 | DOWN | UP | DOWN |
| SNRPA1 | DOWN | DOWN | UP |
| SYNCRIP | DOWN | DOWN | DOWN |
| TNF5F9 | DOWN | DOWN | UP |
| TOMM40 | DOWN | DOWN | UP |
| TPM1 | UP | DOWN | DOWN |
| TSHZ1* | DOWN | DOWN | DOWN |
| TSP4N13 | DOWN | DOWN | DOWN |
| U6AP2 | DOWN | DOWN | UP |
| VAV3 | DOWN | UP | DOWN |
| VDAC2 | DOWN | DOWN | UP |
| ZFAND6 | DOWN | UP | DOWN |

### Example 6

### Cell Type Enrichment Analysis

In addition to the shared induced effects discussed above, each system and platform clearly captured different aspects of GA's impact on the immune system. In mouse splenocytes, genes associated with FOXP3+ regulatory T cells (Tregs), B cells, T cells in general, macrophages, and dendritic cells were significantly modulated upon ex vivo stimulation with GA after prior inoculation (Table 9). In human monocyte (THP-1) cells, upregulated genes were associated with monocytes along with NK cells, dendritic cells, and granulocytes (Table 10). In human PBMCs, genes associated with immune cell types were modulated early in treatment (by month 3), including certain cell types affected in prior systems, but also distinct cell types, such as megakaryocytes and myeloid progenitors (Table 11).

TABLE 9
Immunological cell type enrichment for genes modulated by GA
treatment in mouse splenocytes

| | | | |
|---|---|---|---|
| Dataset Name¹ | Score | p.val | adj.p.val |

a) Upregulated (top 20 results)

| | | | |
|---|---|---|---|
| SC.MEP.BM.rnk | 641.2372079 | 0 | 0 |
| T.8Eff.Sp.OT1.12hr. | 683.0376678 | 0 | 0 |
| LisOva.rnk | | | |
| T.8Eff.Sp.OT1.48hr. | 587.7303361 | 0 | 0 |
| LisOva.rnk | | | |
| T.DPbl.Th.rnk | 659.3284877 | 0 | 0 |
| T.ISP.Th.rnk | 584.8660167 | 0 | 0 |
| preB.FrC.BM.rnk | 620.1611981 | 0 | 0 |
| proB.FrBC.FL.rnk | 595.3446445 | 0 | 0 |
| DC.LC.Sk.rnk | 709.8843317 | 2.89E−15 | 6.96E−14 |
| NK.H..MCMV1.Sp.rnk | 770.77198 | 7.11E−15 | 1.52E−13 |
| DC.103.11b..PolyIC.Lu.rnk | 704.9643794 | 1.44E−14 | 2.65E−13 |
| NK.MCMV1.Sp.rnk | 793.3059033 | 1.51E−14 | 2.65E−13 |
| T.8Eff.Sp.OT1.24hr. | 661.8125467 | 5.97E−14 | 9.61E−13 |
| LisOva.rnk | | | |
| B.GC.Sp.rnk | 602.4630175 | 1.01E−12 | 1.39E−11 |
| Tgd.vg2.24ahi.e17.Th.rnk | 619.9621246 | 9.88E−13 | 1.39E−11 |
| DC.103.11b.24..Lu.rnk | 738.2608372 | 8.91E−12 | 1.15E−10 |
| T.4FP3.25..AA.rnk | 697.5960229 | 2.76E−11 | 3.14E−10 |
| Tgd.vg3.24alo.e17.Th.rnk | 620.789896 | 2.72E−11 | 3.14E−10 |
| proB.FrA.FL.rnk | 578.5853958 | 7.17E−11 | 7.69E−10 |
| preT.DN3B.Th.rnk | 567.9618687 | 1.64E−10 | 1.67E−09 |
| preT.DN3.4.Th.rnk | 560.7279837 | 2.96E−10 | 2.86E−09 |

b) Downregulated (top 20 results)

| | | | |
|---|---|---|---|
| GN.Arth.BM.rnk | 1217.738372 | 0 | 0 |
| SC.CDP.BM.rnk | 1077.505969 | 0 | 0 |
| T.4SP24int.Th.rnk | 1040.246786 | 0 | 0 |
| T.8Nve.Sp.OT1.rnk | 1095.134864 | 0 | 0 |
| proB.CLP.FL.rnk | 1115.01089 | 0 | 0 |
| GN.BM.rnk | 1212.480897 | 1.11E−16 | 3.57E−15 |
| GN.BI.rnk | 1184.87043 | 6.66E−15 | 1.84E−13 |
| GN.UrAc.PC.rnk | 1145.080376 | 5.87E−14 | 1.37E−12 |
| GN.Thio.PC.rnk | 1197.163456 | 6.39E−14 | 1.37E−12 |
| GN.Arth.SynF.rnk | 1160.159783 | 2.72E−11 | 5.25E−10 |
| DC.Ilhilang.103.11blo. | 1112.24759 | 5.07E−08 | 8.90E−07 |
| SLN.rnk | | | |
| DC.Ilhilang.103.11b..SLN.rnk | 1151.229363 | 9.94E−08 | 1.60E−06 |
| DC.8.4.11b..SLN.rnk | 1116.686609 | 1.86E−07 | 2.77E−06 |
| proB.CLP.BM.rnk | 1181.133013 | 1.35E−06 | 1.86E−05 |
| Mo.6C.Il..BM.rnk | 1094.697148 | 1.71E−06 | 2.20E−05 |
| DC.103.11b..LuLN.rnk | 1151.971571 | 2.47E−06 | 2.98E−05 |
| SC.GMP.BM.rnk | 1340.719659 | 1.56E−05 | 0.000164843 |
| SC.MDP.BM.rnk | 1206.224359 | 1.56E−05 | 0.000164843 |
| Mo.6C.Ilint.BI.rnk | 1106.765118 | 1.62E−05 | 0.000164843 |

¹All immunological cell type terminology in Tables 5-7 as defined via Immgen (www.immgen.org/).

TABLE 10
Immunological cell type enrichment for genes upregulated by
GA treatment in human monocytes.

| Dataset Name | Score | p.val | adj.p.val |
|---|---|---|---|
| MONO2.rnk | 108.0701335 | 0 | 0 |
| NKA1.rnk | 97.70225302 | 2.00E−15 | 3.80E−14 |
| MONO1.rnk | 104.5121531 | 8.28E−13 | 1.05E−11 |
| GRAN2.rnk | 97.10283117 | 1.17E−09 | 1.11E−08 |
| GRAN3.rnk | 91.49890345 | 1.27E−07 | 9.40E−07 |
| DENDA2.rnk | 100.4177838 | 1.48E−07 | 9.40E−07 |
| DENDA1.rnk | 111.2455747 | 7.78E−05 | 0.000422143 |
| EOS2.rnk | 94.29494842 | 0.000223937 | 0.001063701 |
| MEGA2.rnk | 101.6956549 | 0.007442566 | 0.031424168 |

TABLE 11
Immunological cell type enrichment for genes modulated by
GA treatment in human PBMC.

| | | | | |
|---|---|---|---|---|
| Dataset Name | Score | p.val | adj.p.val | Genes in list >= threshold |

a) Upregulated, by cluster resulting from consensus k-means
clustering on gene expression profiles
Cluster 0:

| | | | | |
|---|---|---|---|---|
| MEGA2.rnk | 32.42631137 | 1.24E−12 | 4.70E−11 | PTPN18, ILK, RAB27B, FHL1, EPOR, FYN, MAX, |
| | | | | PARVB, TUBB1, TPM1, VCL, TPM4 |

Cluster 2:

| | | | | |
|---|---|---|---|---|
| PRE_BCELL3.rnk | 57.93896297 | 3.98E−08 | 1.51E−06 | IGL@, TOP2A, NUSAP1, IRF4, IGHM, |
| | | | | IGL@, BIRC5 |
| ERY5.rnk | 64.22953395 | 9.88E−07 | 1.88E−05 | IGL@, TOP2A, NUSAP1, IGHM, MYL4, |
| | | | | MKI67, IGL@, H2AFX, RPIP8, GYPC, |
| | | | | BIRC5 |
| ERY2.rnk | 32.83132369 | 8.43E−05 | 0.001067894 | BUB1B, TOP2A, NUSAP1, MCM4, TUBB, |
| | | | | MYL4, CDC6, BIRC5 |
| ERY4.rnk | 69.43612767 | 0.000141687 | 0.001346024 | TOP2A, NUSAP1, TUBB, MYL4, MKI67, |
| | | | | CDC2, RPIP8, GYPC, BIRC5 |
| ERY3.rnk | 84.61769579 | 0.000286865 | 0.002180171 | BUB1B, TOP2A, NUSAP1, TUBB, MYL4, |
| | | | | MKI67, CDC2, RPIP8, GYPC, BIRC5 |
| GRAN1.rnk | 55.17000868 | 0.000557705 | 0.003532132 | IGHG1, IGL@, TOP2A, NUSAP1, IRF4, |
| | | | | IGHM, IGL@, BIRC5 |
| EOS2.rnk | 62.69263216 | 0.002286284 | 0.012411257 | IGL@, IRF4, IGHM, IGL@ |
| BASO1.rnk | 87.29704767 | 0.003219714 | 0.013594346 | IGL@, IGHM, IGL@ |
| ERY1.rnk | 28.25494881 | 0.003043224 | 0.013594346 | BUB1B, NUSAP1, MCM4, TUBB, MS4A2, |
| | | | | BIRC5 |
| MEP.rnk | 30.5446045 | 0.009387769 | 0.035673524 | TOP2A, NUSAP1, MCM4, TUBB, MS4A2, |
| | | | | BIRC5 |

Cluster 4:

| | | | | |
|---|---|---|---|---|
| MEGA2.rnk | 64.4584783 | 4.41E−12 | 1.67E−10 | PARD3, GNB5, C5ORF4, LIMS1, |
| | | | | ACTN1, ITGB3, ITGA2B, GPX1, IGF2BP3, |
| | | | | CLU, LTBP1 |
| MEGA1.rnk | 38.04817949 | 4.66E−09 | 8.86E−08 | HMGA2, LIMS1, ACTN1, ITGB3, |
| | | | | ITGA2B, GPX1, IGF2BP3, LTBP1 |
| ERY2.rnk | 47.24788403 | 4.70E−06 | 5.96E−05 | HMGA2, LIMS1, ACTN1, ITGB3, |
| | | | | ITGA2B, IGF2BP3, CLU, LTBP1 |
| GMP.rnk | 33.12681006 | 0.000142682 | 0.001355477 | ACTN1, GPX1, IGF2BP3, LTBP1 |
| HSC3.rnk | 45.40525942 | 0.000287455 | 0.002184658 | HMGA2, LIMS1, GPX1, IGF2BP3, |
| | | | | HIST1H3D, LTBP1, INPP4B |
| ERY5.rnk | 73.41821315 | 0.000980846 | 0.006212027 | CLCN3, C5ORF4, ITGB3, GPX1, |
| | | | | IGF2BP3, LTBP1 |
| CMP.rnk | 23.1944575 | 0.002214239 | 0.010517637 | HMGA2, IGF2BP3 |
| ERY4.rnk | 77.37714483 | 0.001966408 | 0.010517637 | RAB6B, CLCN3, C5ORF4, GPX1, |
| | | | | IGF2BP3, LTBP1 |
| ERY1.rnk | 45.00063371 | 0.002587112 | 0.010923363 | HMGA2, GPX1, IGF2BP3, MALL, |
| | | | | LTBP1 |
| ERY3.rnk | 90.0166825 | 0.009061135 | 0.031302103 | RAB6B, CLCN3, C5ORF4, GPX1, |
| | | | | IGF2BP3, LTBP1 |
| GRAN2.rnk | 35.17942767 | 0.008750011 | 0.031302103 | ACTN1, ITGB3, GPX1, IGF2BP3, |
| | | | | LTBP1 |

b) Downregulated, by cluster resulting from consensus k-means
clustering on gene expression profiles (as described in methods)
Cluster 0:

| | | | | |
|---|---|---|---|---|
| BCELLA2.rnk | −25.76679646 | 0.000420072 | 0.015962749 | ITSN1, ZNF365, GYG2 |
| BCELLA1.rnk | −19.11170698 | 0.002736809 | 0.025999686 | ITSN1, ANK3, ZNF365, GYG2, EXTL2 |
| BCELLA3.rnk | −20.11301566 | 0.00138472 | 0.025999686 | ITSN1, ZNF365, GYG2 |
| BCELLA4.rnk | −21.51318153 | 0.00253799 | 0.025999686 | ITSN1, LILRA4, GYG2, EXTL2 |

### Discussion

### Examples 5-6

The diversity of the cell types enriched from the list of 1200 genes (B cells, T cells, monocyte progenitors, megakaryocytes) indicates the wide-ranging effects of Copaxone® on the immune system. Similar to what was observed in the splenocyte data, it is difficult to define a small panel of genes to use as quality-control measures against a given Copaxone® lot due to the wide-ranging effect of the drug. Using genome-wide gene-expression arrays, the gene expression from the human PBMC data, mouse splenocytes and human THP-1 monocyte cell line were compared and discovered that although there are genes that are consistently modulated by Copaxone® across all those experimental systems (e.g., IL10), some gene-expression signatures and cell types (e.g., B-cells) were seen only in one system (e.g., human PBMC data) but not as clearly in the other systems (e.g., monocytes and splenocyte data). Looking at genome-wide gene expression signatures can yield a good characterization of the impact of Copaxone® on a single system (e.g., THP-1 monocytes) but well-powered experiments in multiple systems are necessary to characterize the biological impact of Copaxone®.

The generic glatiramer acetate manufactured by Probioglat induced significantly higher expression of CD14 than Copaxone® did (adj. p=0.0135; FIG. 15a) and also induced significantly higher expression of CD40 than Copaxone® did (adj. p=0.0128, FIG. 15b). One key element of Copaxone®'s putative mechanism of action is the ability to shift the immune response from a Th1/Th17 phenotype, typically associated with MS pathoetiology and severity, to a Th2 response type that may mitigate the harmful effects of MS. Across multiple studies, we observed concerning differences between Copaxone® and proposed generics was observed in their impact on key genes associated with maintaining the balance between Th2 and Th1/Th17.

Interleukin-1 beta (IL1B) is a cytokine that stimulates a variety of immune system cells, and may contribute to the development of MS by promoting Th17 cell development. Consistent with these observations, IL1B has also been found to be associated with late disability progression and neurodegeneration in MS. Glatiramer acetate, on the other hand, was reported to significantly reduce interleukin-1beta levels under chronic inflammatory conditions in vitro in human monocytes (p=0.028).

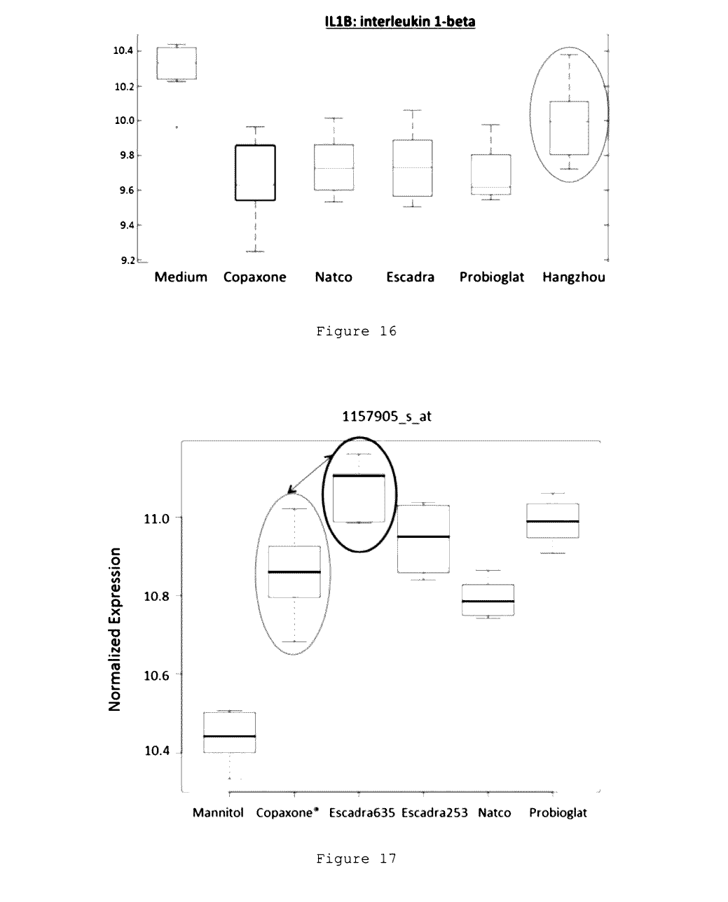

A variety of glatiramoids were significantly less effective than Copaxone® at downregulating IL1B: Copaxone® induced significantly lower IL1B expression than a Natco purported generic in our first mouse splenocyte study (adjusted p=0.043 by ANOVA). Copaxone® was also extremely effective in downregulating IL1B relative to medium (adjusted p=5.72×10-7), while the Hangzhou purported generic (API, China) did not significantly downregulate IL1B relative to medium (adjusted p=0.159) (FIG. 16).

Other Th17-associated genes also seem to be modulated less effectively by purported generics than by Copaxone®. In human monocytes, CD44 was upregulated to a greater extent by Escadra635 than by Copaxone® (adj. p=0.04 by LIMMA at 6 hrs; FIG. 17). CD44 has an established role in Th17 differentiation, specifically: “deletion of CD44 inhibited Th1/Th17 differentiation while simultaneously enhancing Th2/regulatory T cell differentiation.

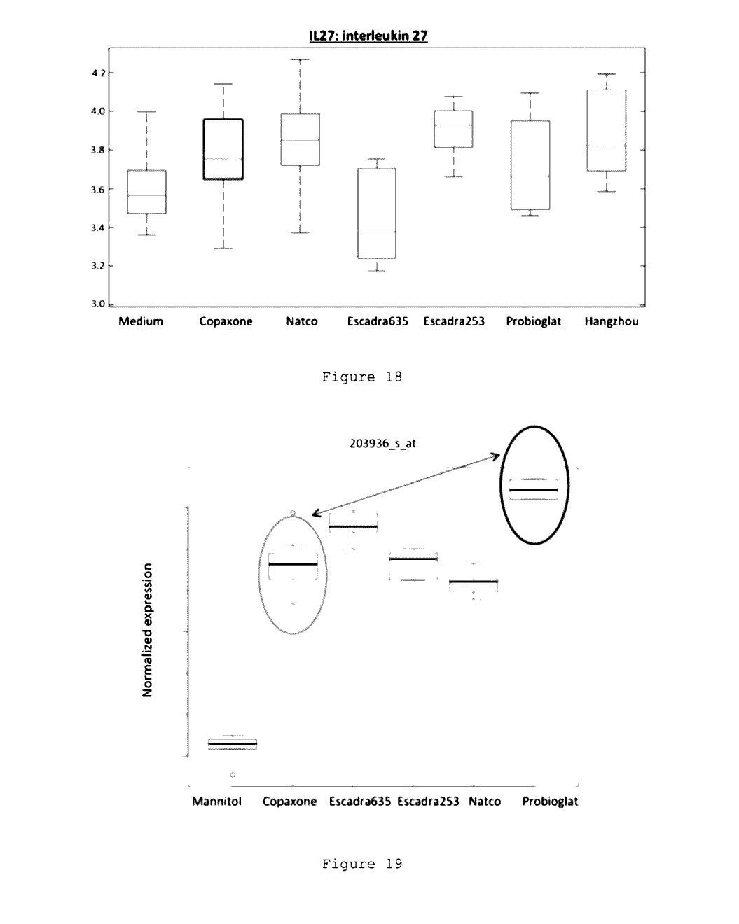

Th17 associated genes also vary from one batch of generics to another. In mouse splenocytes, IL27 was upregulated to significantly different extents by different batches of Escadra, namely Escadra635 and Escadra253 (FIG. 18). IL27 has been proposed to have therapeutic effects in MS, due to its ability to suppress Th17 cells and stimulate neuroprotective factors.

In human monocytes, Probioglat induces significantly higher expression of MMP9 than Copaxone does (Adj. p=2.07×10-5 by LIMMA for Copaxone vs. Probioglat at 6 hrs in human monocytes; FIG. 19). MMP9 is an established biomarker and potential predictor of disease activity in MS. This finding is particularly concerning given the fact that MMP9 facilitates T-cell migration into the CNS, playing a key role in the disruption of the blood-brain barrier (BBB) and thus in the pathogenesis of MS.

### Example 7

### Compositional Differences of GARDPs and GARDSs

Due to its molecular diversity, characterizing even a single, large polypeptide (assuming it could be isolated) would present significant technological and ° scientific barriers making full characterization, as well as a demonstration of active ingredient sameness, impossible.

### Analysis of Additional Purported Generics Reveals Limitations of Focused Gene Panels

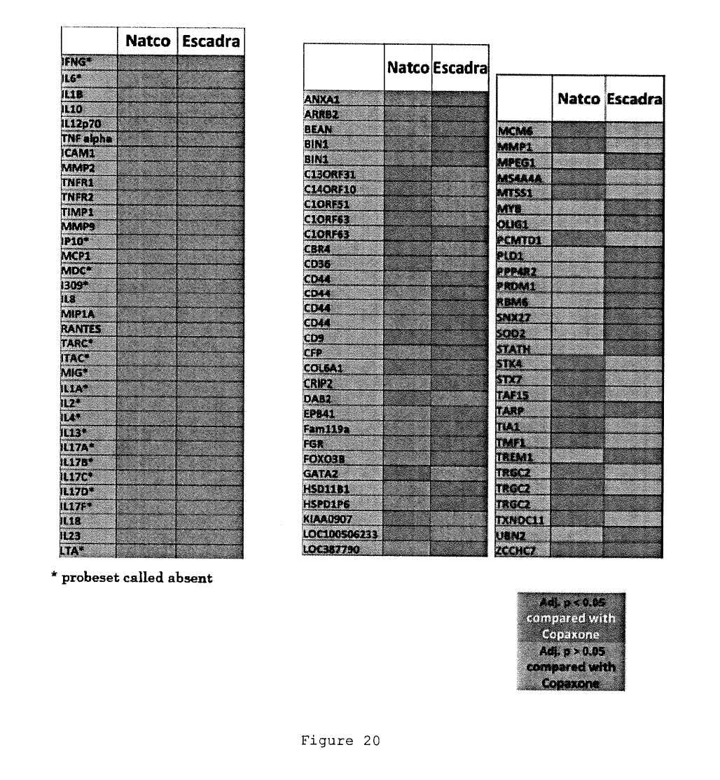

Certain methods have been publicly described in patent application filings by manufacturers seeking to develop purported generics (including International Publication Number WO2008/157697 by Momenta Pharmaceuticals, Inc.), suggesting that glatiramoids can be compared by analyzing a panel comprising only a small subset of proteins and/or the genes coding for those proteins. To evaluate the effectiveness of such comparisons, we applied the same methods used to study Probioglat and Copaxone in human monocytes to also study purported generics Escadra (Raffo, Argentina) and Glatimer (Natco, India). Both purported generics modulated many genes to a significantly different extent than Copaxone. These differentially expressed genes included genes with relevance for multiple sclerosis. For instance, both Natco and Escadra differed significantly from Copaxone in expression of CD9, a component of myelin and a marker of myelinogenic progenitor cells (Sim et al, Nature Biotechnology, 2011). As another example, Escadra differed significantly from Copaxone in expression of CD44, the receptor for hyaluronan which accumulates in demyelinated lesions (Back et al, Nature Medicine, 2005). Despite significant differences such as these between Copaxone and the purported generics in expression of biologically relevant genes, when we examined only the small subset of genes coding for proteins identified in the Momenta patent, there were no significant differences in expression (FIG. 20). This demonstrates that methods focusing on only a small selected subset of genes may miss important differences between two glatiramoids.

### Compositional Differences can be Observed Between Copaxone and Other Generics

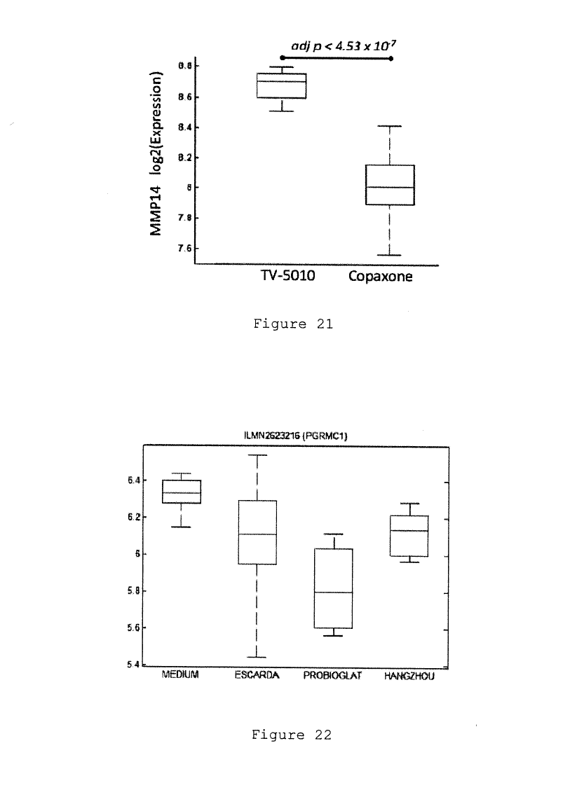

TV-5010 was developed by making slight changes to the manufacturing process for Copaxone®, in order to produce a higher average molecular mass (in the range of 13,500-18,500 Daltons) and investigate whether such a change in molecular mass would be clinically beneficial. Surprisingly, TV-5010 proved toxic in long-term animal studies, inducing fibrosis, nephropathy, increases in eosinophil counts, and severe injection site lesions, including subcutaneous necrosis, vascular necrosis, cavity formation, and inflammation; in some cases these lesions were associated with mortality in both rats and monkeys, possibly related to vascular damage, hemorrhage, thrombus formation, and septicemia. These toxicities were never seen in any of the development programs for Copaxone, and led to the termination of TV-5010's development. Some patients treated with TV-5010 showed injection site reactions and/or developed anti-drug antibodies, but the clinical studies were completed in time to prevent any of the chronic toxicities observed in long-term animal studies from potentially occurring in humans. Because Copaxone® and TV-5010 had many similarities, with the two key differences that (1) TV-5010 had a higher average molecular mass than Copaxone®, and (2) Copaxone® was safe while TV-5010 induced toxicity in long-term animal studies, we sought to determine if there were genes differentially expressed in response to the two medicines that could have predicted toxicity prior to the initiation of long-term animal studies. Having generated gene expression profiles for TV-5010-stimulated splenocytes using the same procedures described above, we applied LIMMA to create a ranked list of genes with significantly different expression levels in response to TV-5010 compared to Copaxone, with both analyzed relative to medium. Among the genes with fold changes greater than 1.5, the gene with the lowest (best) p-value was Matrix Metalloproteinase 14 (MMP14). MMP14 expression was significantly higher in response to TV-5010 than in response to Copaxone® as determined by both ANOVA (adj p<4.53×10-7) and LIMMA (adj P<1.07×10-5) (FIG. 21).

The observed upregulation of MMP14 was striking because MMP14 has been associated with fibrosis and eosinophil-related disorders in the literature, the very same toxicities seen in animals following long-term treatment with TV-5010. MMP14 levels increase to over 250% of control correlating with the pattern of fibrosis manifestation in rats, and MMP14 activity is chronically elevated in a mouse model of dermal fibrosis. Moreover, in patients with the eosinophil-related disorder Eosinophilic Esophagitis (EoE), MMP14 is expressed at a 5.3-fold higher level than in controls. In addition to MMP14, another gene, Signal Transducer and Activator of Transcription 3 (STAT3), which has also been linked to fibrosis, also showed significantly elevated expression in response to TV-5010 relative to Copaxone®. Overall, our findings with TV-5010 lend further support to the utility of mouse splenocyte gene expression studies for predicting drug safety issues.

### Example 8

### Clinical Implications of the Use of Purported Generics

Purported “generics” are not only different from Copaxone® in many ways, but also different from each other, causing differential effects in a variety of pathways modulating immunological processes that could have clinical and biological significance. The differences among “generics” highlight the importance of the manufacturing process for glatiramer acetate, and demonstrate that even slight changes in the manufacturing process can alter the biological properties of the resulting medicine (FIG. 22).

As part of Teva's ongoing commitment to better understand Copaxone®, Teva also has studied Copaxone®'s effect at the level of gene expression across the entire genome (unbiased, without prior hypothesis about the genes for which expression pattern may be altered and without choosing which genes to focus on or study). Genes encode proteins which carry out an array of biological processes in the body, including processes that are essential to the immune system response manifested by exposure to Copaxone®. So-called gene “microarray technology” allows scientists to observe which genes are “turned on” (in scientific terms, “upregulated”), as well as which genes are “turned off” (in scientific terms, “downregulated”) after exposure to various conditions, including stimulation by pharmaceutical products, via measuring the level of mRNA, which is the transitional phase between genes and proteins along the translation process. Teva's gene expression analysis of mouse splenocytes, as well as a human monocyte cell line (THP-1), exposed to Copaxone®, reveals favorable, upregulation of anti-inflammatory genes. These studies provide support for the vast experimental evidence that Copaxone® exerts its well-established therapeutic benefits in part by modulating the immune system to turn on beneficial (i.e. anti-inflammatory and neuro-protective) genes and turn off harmful ones (i.e. pro-inflammatory).

Several competitors have sought approval to market putative generic versions of the drug in foreign countries. Although many of those jurisdictions have required applicants to conduct clinical trials as a condition of approval—and none of those jurisdictions has approved a generic version of Copaxone®—others have not been as careful, and those countries since have allowed purported Copaxone generics to enter the market without proof of the equivalence of the putative generics to Copaxone®.

In particular, the findings for Probioglat discussed below should be carefully addressed, given the serious clinical reports from Mexico following introduction of this purported generic to the market. The complaints expressed by MS patients treated with Probioglat in Mexico included adverse reactions (increased injection site reactions and post injection reactions) and increased occurrence of relapses, even in patients who had been stable for years under Copaxone treatment. These effects may be underlined by a biological imbalance between anti-inflammatory and pro-inflammatory processes, which may be discernible in gene expression differences such as those described herein. The pro-inflammatory signal identified in pre-clinical analyses and its potential association with boosting of the autoimmune mechanisms of disease following switching to Probioglat treatment should raise concerns for the potential health consequences of these differences.

The Teva Patient Support Program in Mexico has an extensive database. Given that patients can switch between branded and purported generic GA, all patients are kept within the database and are provided with patient support services. Interestingly, the database shows differences in patient reports between 2012, when patients in the program were receiving only Copaxone, vs 2013, when patients were receiving both Copaxone® and Probioglat.

The number of relapses reported by patients in Teva's Patient Support Program on a monthly basis during the years 2012 and 2013 i. It is again clear that when Copaxone alone was in use (i.e. throughout 2012) the overall, as well as per-month number of relapses was lower than that reported when Probioglat was present in the market (i.e. throughout 2013).

### Discussion

### Examples 7-8

Gene expression data presented herein for Copaxone® further demonstrates the highly complex mechanism of action of GA, given that many of the GA-induced functional pathways in these experiments coincide with known mechanisms of GA activity in MS patients. Furthermore, the data suggests that other glatiramoids are associated with a significantly altered gene expression profile and thus would probably not behave the same as Copaxone. Most importantly, these biological differences could lead to a marked difference in safety or efficacy profile over the course of chronic treatment.

Probioglat, a purported generic of Copaxone®, has been in commercial use in Mexico since January 2013. The introduction of Probioglat has resulted in a spike of injection site reactions and post injection reactions, as well as occurrence of relapses, even in patients who had been stably in remission for years. Accordingly, several pharmacovigilance reports have been issued by HCPs, and patients expressed complaints in the local media and in Teva's Patient Support Program.

The high occurrence of relapses and adverse events after the treatment switch to Probioglat may be due to an immunological imbalance favoring pro-inflammatory effects, instead of the well recorded beneficial effect that Copaxone induces, reducing pro-inflammation and boosting anti-inflammation.

Overall, similarities in the physicochemical properties of the glatiramer acetate mixture are observed between Copaxone and Probioglat, as well as other purported generics, particularly when using common non-specific analytical methods. However, clear differences are observed between Copaxone and purported generics when applying high resolution methodologies targeted at characterizing functionally relevant elements, e.g. IMMS analyses concomitantly capturing composition, size and charge distribution; and gene expression analyses capturing pro-inflammation distinctly upregulated by purported generics but not Copaxone®.

Gene expression studies thus help explain the biological impact of the physicochemical differences observed, providing insight into some of the factors that may underlie the observed reduction in efficacy and parallel increase in adverse events reported with purported generic glatiramer acetate, notably Probioglat in Mexico.

As part of Teva's ongoing commitment to better understand Copaxone®, Teva studies its effect at the level of gene expression across the entire genome (unbiased, without prior hypothesis about the genes for which expression pattern may be altered and without choosing which genes to focus on or study) in a variety of immunologically relevant model systems, including mouse splenocytes, human monocytes, and peripheral blood mononucleated cells (PBMCs) from MS patients. The genome-wide approach is critical, because two glatiramoids can appear identical based on a small panel of genes, yet differ significantly in their impact on other genes that are potentially highly relevant to safety and/or efficacy. Using multiple model systems is equally critical, since acting as an antigen, Copaxone significantly impacts a variety of immunological cell types. The unbiased approach allows identification of genes and pathways with subtle, yet robust, differential expression patterns following stimulation by different glatiramoids in different experimental contexts. The functionality of identified genes and pathways is then described based on experimental data reported in the peer-reviewed literature. The research has also shown that various model systems capture different aspects of Copaxone's mechanism of action, such that no single cell type or system tested was sufficient to fully characterize the biological impact of this medicine.

### Example 9

### Differences in Gene Expression Induced by Polimunol Versus Branded GA

To identify differences between Copaxone® and differently manufactured glatiramoids, differential gene expression analysis was performed in this study to compare directly between profiles induced by Copaxone® and by the purported generic Polimunol. Based on previous power calculations (using the R package ssize.fdr), to detect differentially-expressed genes with a fold-change between treatments of as low as 1.3 with 80% power the experiment was designed to include six replicates of each condition. The order of sample processing was randomized with respect to treatment in order to avoid creating confounding batch effects.

Cells from a human monocyte cell line (THP-1) were stimulated with either Copaxone®, purported generics from several manufacturers including Polimunol by Synthon, or vehicle control (mannitol) for 6 hours. The 6 hour timepoint was selected because the greatest effects across all treatments were observed at this timepoint in the previous study described above in Example 1. RNA was extracted and expression profiled genome-wide using the Affymetrix U133 Plus 2.0 chip. Three batches of Copaxone® and one batch of Polimunol were comparatively tested in six replicates each. RNA processing was performed by Expression Analysis (NC, USA).

Differentially-expressed probesets were identified across conditions using linear models for microarray data (LIMMA). For comparing Copaxone® (“GA”) and purported generic, comparisons were corrected for mannitol control (i.e., [GA vs mannitol] was compared to [purported generic vs mannitol]). Probesets were filtered by calls of presence on the chip for the relevant samples in the comparison (to be considered present at a given timepoint, a probeset was required to have on average a call of present or marginal across the relevant samples at that timepoint). Probesets were mapped to genes using the U133 Plus 2.0 chip annotation from Affymetrix. Unless otherwise specified, all probesets called present for a gene showed the effect discussed above; where all multiple probesets were present for a gene, p values are reported for the most significant probeset.

Upregulated and downregulated genes were analyzed separately for pathway enrichment, using DAVID [Huang, D. W., Sherman, B. T. & Lempicki, R. A. Bioinformatics enrichment tools: paths toward the comprehensive functional analysis of large gene lists. Nucleic Acids Res. 37, 1-13 (2009)]. Pathway enrichment results were visualized using volcano plots, plotting −log p-values versus enrichment scores. For GA MOA, to obtain top-gene lists of appropriate size (tens-hundreds) for use with DAVID, an absolute-value fold-change cutoff of 1.5 and p-value cutoff of 1e-3 were utilized to obtain gene lists for pathway enrichment. For comparisons between GA and Polimunol, upregulated or downregulated genes with FDR-adjusted p-values<0.05 were used for pathway enrichment. DAVID runs were conducted on Sep. 12, 2014. Please note that the GO databases are updated daily (as noted on the GO site:www.geneontology.org/GO.downloads.ontology.shtml) and therefore performing the same enrichments on the same genesets may yield slightly varying results depending on the rundate. Pathway p-values may change slightly between runs conducted at different times; the overall picture of enriched pathways, however, is expected to remain consistent.

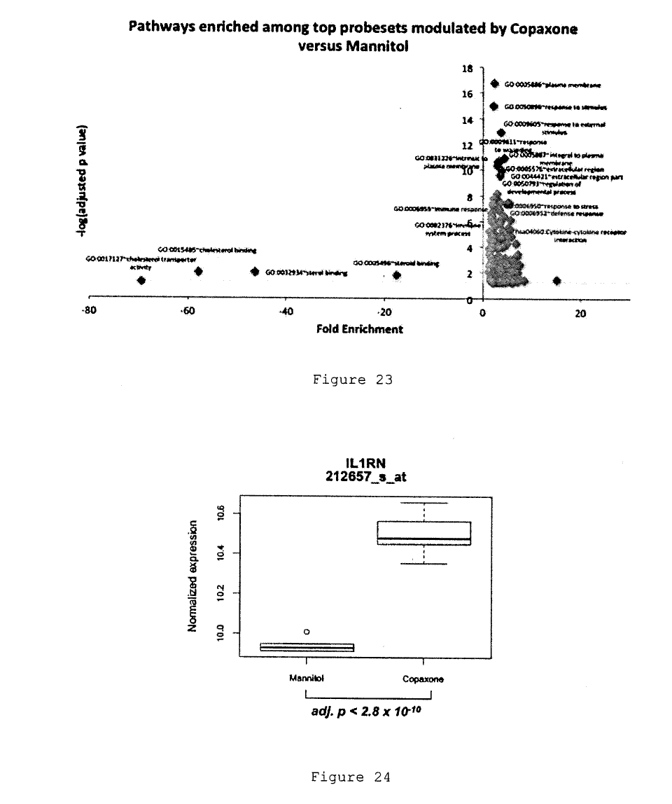

Genes differentially expressed between Copaxone® and mannitol control are enriched with a variety of pathways in Kegg and GO (molecular function (MF), biological process (BP), and cellular component (CC)), many of which affect immunological responses, demonstrating Copaxone's complex mechanism of action, as shown in FIG. 23. Pathways enriched among top probesets modulated by Copaxone versus mannitol control are shown in Table 12. More than 80% of the pathways that enriched among top upregulated genes in the previous study were also enriched among top upregulated genes in the current study.

As an example at the individual gene level, IL1RN was significantly upregulated (adjusted p<2.8e-10), as shown in FIG. 24, which is consistent with the previous study described in Example 1 above. This gene encodes for the protein IL-1ra, which inhibits the activities of pro-inflammatory cytokines IL-1a and IL-1b.

The probeset for IL10, which had been observed to be upregulated by Copaxone® above in Example 1, was called absent in this experiment, but was nominally upregulated (p<0.029). However, the IL10 receptor IL10RA was significantly upregulated (for the single probeset for this gene, which was called present) (adjusted p<2.8e-14).

Genes analyzed for pathway enrichment (Table 12), using DAVID (conducted Sep. 12, 2014). Performing the same enrichments on the same genesets may yield slightly varying results depending on the run date (GO databases are updated daily: www.geneontology.org/GO.downloads.ontology.shtml).

TABLE 12
Human monocyte study: pathways significantly enriched among genes significantly upregulated by Copaxone ® relative to mannitol at 6 hours

| | | | | | | | | Fold | | | |
|---|---|---|---|---|---|---|---|---|---|---|---|
| Category | Term | Count | % | Pvalue | List Total | Pop Hits | Pop Total | Enrichment | Bonferroni | Benjamini | FDR |
| GOTERM_{—} | GO:005886~plasma | 121 | 35.17 | 6.53E−20 | 305 | 1699 | 9576 | 2.236 | 1.78E−17 | 1.78E−17 | 8.60E−17 |
| CC_ALL | membrane | | | | | | | | | | |
| GOTERM_{—} | GO:0050895~response | 127 | 36.92 | 4.65E−19 | 291 | 1815 | 8731 | 2.099 | 1.01E−15 | 1.01E−15 | 8.07E−16 |
| BP_ALL | to stimulus | | | | | | | | | | |
| GOTERM_{—} | GO:0009605~response | 57 | 16.57 | 9.39E−17 | 291 | 492 | 8731 | 3.476 | 2.42E−13 | 1.21E−13 | 1.89E−13 |
| BP_ALL | to external stimulus | | | | | | | | | | |
| GOTERM_{—} | GO:0009611~response | 40 | 11.63 | 1.44E−14 | 291 | 281 | 8731 | 4.271 | 3.15E−11 | 1.05E−11 | 2.51E−11 |
| BP_ALL | to wounding | | | | | | | | | | |
| GOTERM_{—} | GO:0005887~integral | 53 | 15.41 | 1.31E−13 | 305 | 528 | 9576 | 3.152 | 3.57E−11 | 1.79E−11 | 1.73E−10 |
| CC_ALL | to plasma membrane | | | | | | | | | | |
| GOTERM_{—} | GO:0031226~intrinsic | 53 | 15.41 | 2.60E−13 | 305 | 537 | 9576 | 3.099 | 7.08E−11 | 2.36E−11 | 3.42E−10 |
| CC_ALL | to plasma membrane | | | | | | | | | | |
| GOTERM_{—} | GO:0005576~extracellular | 59 | 17.15 | 6.56E−13 | 305 | 662 | 9576 | 2.798 | 1.79E−10 | 4.46E−11 | 8.64E−10 |
| CC_ALL | region | | | | | | | | | | |
| GOTERM_{—} | GO:0044421~extracellular | 39 | 11.34 | 1.93E−12 | 305 | 324 | 9576 | 3.779 | 5.26E−10 | 1.05E−10 | 2.54E−09 |
| CC_ALL | region part | | | | | | | | | | |
| GOTERM_{—} | GO:0050793~regulation | 46 | 13.37 | 4.84E−13 | 291 | 406 | 8731 | 3.399 | 1.06E−09 | 2.64E−10 | 8.41E−10 |
| BP_ALL | of developmental process | | | | | | | | | | |
| GOTERM_{—} | GO:0051239~regulation | 50 | 14.53 | 1.43E−11 | 291 | 517 | 8731 | 2.902 | 3.12E−08 | 6.23E−09 | 2.48E−08 |
| BP_ALL | of multicellular | | | | | | | | | | |
| | organismal process | | | | | | | | | | |
| GOTERM_{—} | GO:0004872~receptor | 54 | 15.70 | 4.34E−11 | 297 | 637 | 9375 | 2.676 | 2.57E−08 | 1.28E−08 | 6.40E−08 |
| MF_ALL | activity | | | | | | | | | | |
| GOTERM_{—} | GO:0060089~molecular | 68 | 19.77 | 2.37E−11 | 297 | 915 | 9375 | 2.346 | 1.40E−08 | 1.40E−08 | 3.49E−08 |
| MF_ALL | transducer activity | | | | | | | | | | |
| GOTERM_{—} | GO:0004871~signal | 68 | 19.77 | 2.37E−11 | 297 | 915 | 9375 | 2.346 | 1.40E−08 | 1.40E−08 | 3.49E−08 |
| MF_ALL | transducer activity | | | | | | | | | | |
| GOTERM_{—} | GO:004888~transmembrane | 36 | 10.47 | 1.44E−10 | 297 | 325 | 9375 | 3.497 | 8.53E−08 | 2.84E−08 | 2.12E−07 |
| MF_ALL | receptor activity | | | | | | | | | | |
| GOTERM_{—} | GO:0001568~blood | 25 | 7.27 | 8.49E−11 | 291 | 148 | 8731 | 5.068 | 1.86E−07 | 3.09E−08 | 1.48E−07 |
| BP_ALL | vessel development | | | | | | | | | | |
| GOTERM_{—} | GO:0044459~plasma | 73 | 21.22 | 7.32E−10 | 305 | 1095 | 9576 | 2.093 | 1.99E−07 | 3.32E−08 | 9.63E−07 |
| CC_ALL | membrane part | | | | | | | | | | |
| GOTERM_{—} | GO:0048731~system | 83 | 24.13 | 1.24E−10 | 291 | 1231 | 8731 | 2.023 | 2.70E−07 | 3.38E−08 | 2.15E−07 |
| BP_ALL | development | | | | | | | | | | |
| GOTERM_{—} | GO:0048514~blood | 23 | 6.69 | 1.43E−10 | 291 | 127 | 8731 | 5.434 | 3.12E−07 | 3.46E−08 | 2.48E−07 |
| BP_ALL | vessel morphogenesis | | | | | | | | | | |
| GOTERM_{—} | GO:0001944~vasculature | 25 | 7.27 | 1.14E−10 | 291 | 150 | 8731 | 5.001 | 2.48E−07 | 3.54E−08 | 1.97E−07 |
| BP_ALL | development | | | | | | | | | | |
| GOTERM_{—} | GO:0006950~response | 76 | 22.09 | 2.20E−10 | 291 | 1089 | 8731 | 2.094 | 4.80E−07 | 4.80E−08 | 3.81E−07 |
| BP_ALL | to stress | | | | | | | | | | |
| GOTERM_{—} | GO:0006955~immune | 38 | 11.05 | 4.63E−10 | 291 | 356 | 8731 | 3.203 | 1.01E−06 | 9.19E−08 | 8.04E−07 |
| BP_ALL | response | | | | | | | | | | |
| GOTERM_{—} | GO:0006952~defense | 35 | 10.17 | 6.58E−10 | 291 | 311 | 8731 | 3.377 | 1.44E−06 | 1.20E−07 | 1.14E−06 |
| BP_ALL | response | | | | | | | | | | |
| GOTERM_{—} | GO:0032501~multicellular | 112 | 32.56 | 1.21E−09 | 291 | 1991 | 8731 | 1.688 | 2.64E−06 | 2.03E−07 | 2.10E−06 |
| BP_ALL | organismal process | | | | | | | | | | |
| GOTERM_{—} | GO:0006954~inflammatory | 25 | 7.27 | 2.58E−09 | 291 | 174 | 8731 | 4.311 | 5.64E−06 | 3.76E−07 | 4.48E−06 |
| BP_ALL | response | | | | | | | | | | |
| GOTERM_{—} | GO:0007275~multicellular | 93 | 27.03 | 2.50E−09 | 291 | 1544 | 8731 | 1.807 | 5.47E−06 | 3.90E−07 | 4.35E−06 |
| BP_ALL | organismal development | | | | | | | | | | |
| GOTERM_{—} | GO:0040011~locomotion | 29 | 8.43 | 4.62E−09 | 291 | 238 | 8731 | 3.656 | 1.01E−05 | 6.30E−07 | 8.02E−06 |
| BP_ALL | | | | | | | | | | | |
| GOTERM_{—} | GO:0032502~developmental | 100 | 29.07 | 5.35E−09 | 291 | 1738 | 8731 | 1.726 | 1.17E−05 | 6.49E−07 | 9.30E−06 |
| BP_ALL | process | | | | | | | | | | |
| GOTERM_{—} | GO:0048545~response | 19 | 5.52 | 5.22E−09 | 291 | 102 | 8731 | 5.589 | 1.14E−05 | 6.71E−07 | 9.07E−06 |
| BP_ALL | to steroid hormone | | | | | | | | | | |
| | stimulus | | | | | | | | | | |
| GOTERM_{—} | GO:0005615~extracellular | 27 | 7.85 | 2.53E−08 | 305 | 235 | 9576 | 3.607 | 6.89E−06 | 9.84E−07 | 3.34E−05 |
| CC_ALL | space | | | | | | | | | | |
| GOTERM_{—} | GO:0048856~anatomical | 84 | 24.42 | 8.74E−09 | 291 | 1366 | 8731 | 1.845 | 1.91E−05 | 1.01E−06 | 1.52E−05 |
| BP_ALL | structure development | | | | | | | | | | |
| GOTERM_{—} | GO:0048583~regulation | 30 | 8.72 | 1.29E−08 | 291 | 265 | 8731 | 3.397 | 2.82E−05 | 1.41E−06 | 2.24E−05 |
| BP_ALL | of response to | | | | | | | | | | |
| | stimulus | | | | | | | | | | |
| GOTERM_{—} | GO:0002376~immune | 47 | 13.66 | 1.94E−08 | 291 | 578 | 8731 | 2.440 | 4.25E−05 | 2.02E−06 | 3.38E−05 |
| BP_ALL | system process | | | | | | | | | | |
| GOTERM_{—} | GO:0031012~extracellular | 18 | 5.23 | 8.69E−08 | 305 | 113 | 9576 | 5.001 | 2.36E−05 | 2.95E−06 | 0.0011434 |
| CC_ALL | matrix | | | | | | | | | | |
| GOTERM_{—} | GO:0001525~angiogenesis | 17 | 4.94 | 4.00E−08 | 291 | 91 | 8731 | 5.605 | 8.73E−05 | 3.97E−06 | 6.95E−05 |
| BP_ALL | | | | | | | | | | | |
| GOTERM_{—} | GO:0051707~response | 22 | 6.40 | 5.14E−08 | 291 | 158 | 8731 | 4.178 | 0.00011219 | 4.88E−06 | 8.92E−05 |
| BP_ALL | to other organism | | | | | | | | | | |
| GOTERM_{—} | GO:0016477~cell | 22 | 6.40 | 6.42E−08 | 291 | 160 | 8731 | 4.125 | 0.00014011 | 5.84E−06 | 0.00011144 |
| BP_ALL | migration | | | | | | | | | | |
| GOTERM_{—} | GO:0016020~membrane | 169 | 49.13 | 1.96E−07 | 305 | 3915 | 9576 | 1.355 | 5.33E−05 | 5.93E−06 | 0.00025814 |
| CC_ALL | | | | | | | | | | | |
| KEGG_{—} | hsa04060: Cytokine- | 19 | 5.52 | 8.99E−08 | 123 | 109 | 3163 | 4.483 | 9.53E−06 | 9.53E−06 | 0.00010084 |
| PATHWAY | cytokine receptor | | | | | | | | | | |
| | interaction | | | | | | | | | | |
| GOTERM_{—} | GO:0009653~anatomical | 50 | 14.53 | 1.18E−07 | 291 | 675 | 8731 | 2.222 | 0.00025871 | 9.58E−06 | 0.00020578 |
| BP_ALL | structure morphogenesis | | | | | | | | | | |
| GOTERM_{—} | GO:0042221~response | 56 | 16.28 | 1.17E−07 | 291 | 802 | 8731 | 2.095 | 0.00025627 | 9.86E−06 | 0.00020384 |
| BP_ALL | to chemical stimulus | | | | | | | | | | |
| GOTERM_{—} | GO:0010033~response | 39 | 11.34 | 1.26E−07 | 291 | 457 | 8731 | 2.560 | 0.0002761 | 9.86E−06 | 0.00021962 |
| BP_ALL | to organic substance | | | | | | | | | | |
| GOTERM_{—} | GO:0048513~organ | 62 | 18.02 | 1.13E−07 | 291 | 532 | 8731 | 1.996 | 0.00024675 | 9.87E−06 | 0.00019627 |
| BP_ALL | development | | | | | | | | | | |
| GOTERM_{—} | GO:0048519~negative | 74 | 21.51 | 1.50E−07 | 291 | 1214 | 8731 | 1.829 | 0.00032738 | 1.13E−05 | 0.00026041 |
| BP_ALL | regulation of biological | | | | | | | | | | |
| | process | | | | | | | | | | |
| GOTERM_{—} | GO:0032101~regulation | 15 | 4.36 | 1.86E−07 | 291 | 77 | 8731 | 5.845 | 0.00040597 | 1.35E−05 | 0.00032294 |
| BP_ALL | of response to | | | | | | | | | | |
| | external stimulus | | | | | | | | | | |
| GOTERM_{—} | GO:0065008~regulation | 57 | 16.57 | 1.97E−07 | 291 | 836 | 8731 | 2.046 | 0.00042941 | 1.39E−05 | 0.0003416 |
| BP_ALL | of biological quality | | | | | | | | | | |
| GOTERM_{—} | GO:0005578~proteinaceous | 16 | 4.65 | 5.27E−07 | 305 | 100 | 9576 | 5.023 | 0.00014321 | 1.43E−05 | 0.00069309 |
| CC_ALL | extracellular matrix | | | | | | | | | | |
| GOTERM_{—} | GO:0048646~anatomical | 25 | 7.27 | 2.19E−07 | 291 | 218 | 8731 | 3.441 | 0.00047903 | 1.50E−05 | 0.00038107 |
| BP_ALL | structure formation | | | | | | | | | | |
| | involved in | | | | | | | | | | |
| | morphogenesis | | | | | | | | | | |
| GOTERM_{—} | GO:0048870~cell | 22 | 6.40 | 2.49E−07 | 291 | 173 | 8731 | 3.815 | 0.00054426 | 1.65E−05 | 0.00043298 |
| BP_ALL | motility | | | | | | | | | | |
| GOTERM_{—} | GO:0051674~localization | 22 | 6.40 | 2.49E−07 | 291 | 173 | 8731 | 3.815 | 0.00054426 | 1.65E−05 | 0.00043298 |
| BP_ALL | of cell | | | | | | | | | | |
| GOTERM_{—} | GO:0051094~positive | 22 | 5.40 | 2.75E−07 | 291 | 174 | 8731 | 3.794 | 0.00060061 | 1.77E−05 | 0.00047782 |
| BP_ALL | regulation of | | | | | | | | | | |
| | developmental process | | | | | | | | | | |
| GOTERM_{—} | GO:0031224~intrinsic | 123 | 35.76 | 1.13E−06 | 305 | 2645 | 9576 | 1.460 | 0.00030837 | 2.80E−05 | 0.00149255 |
| CC_ALL | to membrane | | | | | | | | | | |
| GOTERM_{—} | GO:0030154~cell | 58 | 16.86 | 5.18E−07 | 291 | 883 | 8731 | 1.971 | 0.00113121 | 3.23E−05 | 0.00090018 |
| BP_ALL | differentiation | | | | | | | | | | |
| GOTERM_{—} | GO:0007166~cell | 48 | 13.35 | 5.38E−07 | 291 | 667 | 8731 | 2.159 | 0.00117393 | 3.26E−05 | 0.0009342 |
| BP_ALL | surface receptor linked | | | | | | | | | | |
| | signal transduction | | | | | | | | | | |
| GOTERM_{—} | GO:0019955~cytokine | 13 | 3.78 | 3.43E−07 | 297 | 61 | 9375 | 6.727 | 0.00020278 | 5.07E−05 | 0.00050503 |
| MF_ALL | binding | | | | | | | | | | |
| GOTERM_{—} | GO:0042060~wound | 16 | 4.65 | 9.16E−07 | 291 | 100 | 8731 | 4.801 | 0.00199804 | 5.41E−05 | 0.00159067 |
| BP_ALL | healing | | | | | | | | | | |
| GOTERM_{—} | GO:0006928~cell | 27 | 7.85 | 1.17E−06 | 291 | 273 | 8731 | 2.967 | 0.00254332 | 6.70E−05 | 0.00202532 |
| BP_ALL | motion | | | | | | | | | | |
| GOTERM_{—} | GO:0022603~regulation | 18 | 5.23 | 1.40E−06 | 291 | 131 | 8731 | 4.123 | 0.00305304 | 7.84E−05 | 0.00243184 |
| BP_ALL | of anatomical | | | | | | | | | | |
| | structure morphogenesis | | | | | | | | | | |
| GOTERM_{—} | GO:0048518~positive | 76 | 22.09 | 1.48E−06 | 291 | 1335 | 8731 | 1.708 | 0.00323049 | 7.89E−05 | 0.00257341 |
| BP_ALL | regulation of | | | | | | | | | | |
| | biological process | | | | | | | | | | |
| GOTERM_{—} | GO:0045597~positive | 19 | 5.52 | 1.46E−06 | 291 | 146 | 8731 | 3.905 | 0.00318974 | 7.99E−05 | 0.0025409 |
| BP_ALL | regulation of cell | | | | | | | | | | |
| | differentiation | | | | | | | | | | |
| GOTERM_{—} | GO:0048869~cellular | 59 | 17.15 | 1.61E−06 | 291 | 937 | 8731 | 1.889 | 0.00351577 | 8.39E−05 | 0.00280106 |
| BP_ALL | developmental process | | | | | | | | | | |
| GOTERM_{—} | GO:0045595~regulation | 28 | 8.14 | 1.90E−06 | 291 | 298 | 8731 | 2.819 | 0.00413858 | 9.64E−05 | 0.00329828 |
| BP_ALL | of cell differentiation | | | | | | | | | | |
| GOTERM_{—} | GO:0065007~biological | 189 | 54.94 | 2.27E−06 | 291 | 4499 | 8731 | 1.260 | 0.00493883 | 0.00011252 | 0.00393762 |
| BP_ALL | regulation | | | | | | | | | | |
| GOTERM_{—} | GO:0009607~response | 24 | 6.98 | 2.33E−06 | 291 | 231 | 8731 | 3.117 | 0.00508171 | 0.00011321 | 0.00405183 |
| BP_ALL | to biotic stimulus | | | | | | | | | | |
| GOTERM_{—} | GO:0016021~integral | 117 | 34.01 | 7.05E−06 | 305 | 2564 | 9576 | 1.433 | 0.00191674 | 0.00015987 | 0.00928437 |
| CC_ALL | to membrane | | | | | | | | | | |
| GOTERM_{—} | GO:0050789~regulation | 181 | 52.62 | 6.79E−06 | 291 | 4312 | 8731 | 1.259 | 0.01472889 | 0.00032252 | 0.01180067 |
| BP_ALL | of biological process | | | | | | | | | | |
| GOTERM_{—} | GO:0051384~response | 10 | 2.91 | 7.38E−06 | 291 | 42 | 8731 | 7.144 | 0.01599922 | 0.0003431 | 0.01282663 |
| BP_ALL | to glucocorticoid stimulus | | | | | | | | | | |
| GOTERM_{—} | GO:0002682~regulation | 23 | 6.69 | 1.05E−05 | 291 | 235 | 8731 | 2.937 | 0.02269405 | 0.00047813 | 0.01825541 |
| BP_ALL | of immune system process | | | | | | | | | | |
| GOTERM_{—} | GO:0031960~response | 10 | 2.91 | 1.11E−05 | 291 | 44 | 8731 | 6.819 | 0.02389824 | 0.00049352 | 0.01923579 |
| BP_ALL | to corticosteroid stimulus | | | | | | | | | | |
| GOTERM_{—} | GO:0007610~behavior | 22 | 6.40 | 1.34E−05 | 291 | 221 | 8731 | 2.987 | 0.02894023 | 0.00058717 | 0.02335375 |
| BP_ALL | | | | | | | | | | | |
| GOTERM_{—} | GO:0009617~response | 14 | 4.07 | 1.39E−05 | 291 | 95 | 8731 | 4.422 | 0.02982868 | 0.0005936 | 0.02408158 |
| BP_ALL | to bacterium | | | | | | | | | | |
| GOTERM_{—} | GO:0007626~locomotory | 17 | 4.94 | 1.51E−05 | 291 | 140 | 8731 | 3.643 | 0.03243116 | 0.00063381 | 0.02621736 |
| BP_ALL | behavior | | | | | | | | | | |
| GOTERM_{—} | GO:0048523~negative | 64 | 18.60 | 1.57E−05 | 291 | 1127 | 8731 | 1.704 | 0.03368346 | 0.00064628 | 0.02724712 |
| BP_ALL | regulation of | | | | | | | | | | |
| | cellular process | | | | | | | | | | |
| GOTERM_{—} | GO:0043627~response | 11 | 3.20 | 2.08E−05 | 291 | 59 | 8731 | 5.594 | 0.04438121 | 0.00084032 | 0.03609816 |
| BP_ALL | to estrogen stimulus | | | | | | | | | | |
| GOTERM_{—} | GO:0048878~chemical | 23 | 6.69 | 2.33E−05 | 291 | 247 | 8731 | 2.794 | 0.0495174 | 0.00092295 | 0.0403827 |
| BP_ALL | homeostasis | | | | | | | | | | |
| GOTERM_{—} | GO:0019838~growth | 11 | 3.20 | 8.31E−05 | 297 | 56 | 9375 | 6.200 | 0.0049076 | 0.00098345 | 0.01225035 |
| MF_ALL | factor binding | | | | | | | | | | |
| GOTERM_{—} | GO:0070482~response | 13 | 3.78 | 2.74E−05 | 291 | 87 | 8731 | 4.483 | 0.05817137 | 0.00106964 | 0.04765395 |
| BP_ALL | to oxygen levels | | | | | | | | | | |
| GOTERM_{—} | GO:0000262~cell | 41 | 11.92 | 5.68E−05 | 305 | 662 | 9576 | 1.945 | 0.01533071 | 0.00118767 | 0.07473585 |
| CC_ALL | fraction | | | | | | | | | | |
| GOTERM_{—} | GO:0005626~insoluble | 34 | 9.88 | 6.61E−05 | 305 | 508 | 9576 | 2.101 | 0.017809867 | 0.00128276 | 0.08692732 |
| CC_ALL | fraction | | | | | | | | | | |
| GOTERM_{—} | GO:0006935~chemotaxis | 13 | 3.78 | 3.46E−05 | 291 | 89 | 8731 | 4.383 | 0.07280814 | 0.00132535 | 0.06020426 |
| BP_ALL | | | | | | | | | | | |
| GOTERM_{—} | GO:0042330~taxis | 13 | 3.78 | 3.46E−05 | 291 | 89 | 8731 | 4.383 | 0.07280814 | 0.00132535 | 0.06010426 |
| BP_ALL | | | | | | | | | | | |
| GOTERM_{—} | GO:0009968~negative | 16 | 4.65 | 3.90E−05 | 291 | 135 | 8731 | 3.556 | 0.08171162 | 0.00146864 | 0.06777345 |
| BP_ALL | regulation of signal | | | | | | | | | | |
| GOTERM_{—} | GO:0014070~response | 12 | 3.49 | 4.26E−05 | 291 | 77 | 8731 | 4.676 | 0.0888131 | 0.00152355 | 0.07394353 |
| BP_ALL | to organic cyclic | | | | | | | | | | |
| | substance | | | | | | | | | | |
| GOTERM_{—} | GO:0007165~signal | 74 | 21.51 | 4.14E−05 | 291 | 1411 | 8731 | 1.574 | 0.08646755 | 0.00153165 | 0.07190035 |
| BP_ALL | transduction | | | | | | | | | | |
| GOTERM_{—} | GO:0048584~positive | 16 | 4.65 | 4.26E−05 | 291 | 136 | 8731 | 3.530 | 0.08879912 | 0.00154866 | 0.07393134 |
| BP_ALL | regulation of | | | | | | | | | | |
| | response to | | | | | | | | | | |
| | stimulus | | | | | | | | | | |
| GOTERM_{—} | GO:0009719~response | 22 | 6.40 | 5.26E−05 | 291 | 242 | 8731 | 2.728 | 0.10344839 | 0.00184978 | 0.09125557 |
| BP_ALL | to endogenous stimulus | | | | | | | | | | |
| GOTERM_{—} | GO:0045765~regulation | 8 | 2.33 | 6.83E−05 | 291 | 32 | 8731 | 7.501 | 0.1386353 | 0.00236604 | 0.11862156 |
| BP_ALL | of angiogenesis | | | | | | | | | | |
| GOTERM_{—} | GO:0009991~response | 16 | 4.65 | 7.04E−05 | 291 | 142 | 8731 | 3.381 | 0.14260665 | 0.00240115 | 0.12229249 |
| BP_ALL | to extracellular | | | | | | | | | | |
| | stimulus | | | | | | | | | | |
| GOTERM_{—} | GO:0048522~positive | 66 | 19.19 | 7.28E−05 | 291 | 1234 | 8731 | 1.605 | 0.14701919 | 0.00244344 | 0.12639105 |
| BP_ALL | regulation of | | | | | | | | | | |
| | cellular process | | | | | | | | | | |
| GOTERM_{—} | GO:0005624~membrane | 32 | 9.30 | 0.0001466 | 305 | 484 | 9576 | 2.076 | 0.03909456 | 0.00265509 | 0.19280628 |
| CC_ALL | fraction | | | | | | | | | | |
| GOTERM_{—} | GO:0044425~membrane | 140 | 40.70 | 0.00016676 | 305 | 3420 | 9576 | 1.285 | 0.04435026 | 0.00283122 | 0.21929353 |
| CC_ALL | part | | | | | | | | | | |
| GOTERM_{—} | GO:0001666~response | 12 | 3.49 | 8.62E−05 | 291 | 83 | 8731 | 4.338 | 0.17161099 | 0.00284855 | 0.14962549 |
| BP_ALL | to hypoxia | | | | | | | | | | |
| GOTERM_{—} | GO:0050776~regulation | 15 | 4.36 | 9.15E−05 | 291 | 123 | 8731 | 3.489 | 0.18106373 | 0.00297689 | 0.15873903 |
| BP_ALL | of immune response | | | | | | | | | | |
| GOTERM_{—} | GO:0010648~negative | 16 | 4.65 | 9.69E−05 | 291 | 146 | 8731 | 3.288 | 0.19077735 | 0.00306314 | 0.16821347 |
| BP_ALL | regulation of cell | | | | | | | | | | |
| | communication | | | | | | | | | | |
| GOTERM_{—} | GO:0048585~negative | 10 | 2.91 | 9.62E−05 | 291 | 57 | 8731 | 5.264 | 0.19954902 | 0.00308588 | 0.16700917 |
| BP_ALL | regulation of response | | | | | | | | | | |
| | to stimulus | | | | | | | | | | |
| GOTERM_{—} | GO:0031099~regeneration | 8 | 2.33 | 0.00010316 | 291 | 34 | 8731 | 7.060 | 0.20173321 | 0.00321357 | 0.17903588 |
| BP_ALL | | | | | | | | | | | |
| GOTERM_{—} | GO:0009725~response | 20 | 5.81 | 0.0001064 | 291 | 217 | 8731 | 2.765 | 0.20736027 | 0.0032677 | 0.18465182 |
| BP_ALL | to hormone stimulus | | | | | | | | | | |
| GOTERM_{—} | GO:0050801~ion | 19 | 5.52 | 0.00011383 | 291 | 200 | 8731 | 2.850 | 0.22011692 | 0.00344698 | 0.19753113 |
| BP_ALL | homeostasis | | | | | | | | | | |
| GOTERM_{—} | GO:0007155~cell | 26 | 7.56 | 0.00011726 | 291 | 334 | 8731 | 2.336 | 0.22594061 | 0.00350216 | 0.20348045 |
| BP_ALL | adhesion | | | | | | | | | | |
| GOTERM_{—} | GO:0022610~biological | 26 | 7.56 | 0.00012311 | 291 | 335 | 8731 | 2.329 | 0.23576926 | 0.00362699 | 0.21362257 |
| BP_ALL | adhesion | | | | | | | | | | |
| GOTERM_{—} | GO:0006873~cellular | 18 | 5.23 | 0.00016537 | 291 | 163 | 8731 | 2.873 | 0.30315535 | 0.00480433 | 0.28685303 |
| BP_ALL | ion homeostasis | | | | | | | | | | |
| GOTERM_{—} | GO:0055082~cellular | 18 | 5.23 | 0.00017635 | 291 | 189 | 8731 | 2.857 | 0.31967946 | 0.00505548 | 0.30588293 |
| BP_ALL | chemical homeostatis | | | | | | | | | | |
| GOTERM_{—} | GO:0002684~positive | 15 | 4.36 | 0.00018897 | 291 | 138 | 8731 | 3.261 | 0.33816687 | 0.00534594 | 0.32772523 |
| BP_ALL | regulation of immune | | | | | | | | | | |
| | system process | | | | | | | | | | |
| GOTERM_{—} | GO:0002703~regulation | 8 | 2.33 | 0.00021596 | 291 | 38 | 8731 | 6.317 | 0.37607009 | 0.00595331 | 0.37446511 |
| BP_ALL | of leukocyte | | | | | | | | | | |
| | mediated immunity | | | | | | | | | | |
| GOTERM_{—} | GO:0032496~response | 9 | 2.62 | 0.00021432 | 291 | 50 | 8731 | 5.401 | 0.37382152 | 0.00598356 | 0.37161469 |
| BP_ALL | to lipopolysaccharide | | | | | | | | | | |
| KEGG_{—} | hsa04640: Hematopoietic | 9 | 2.62 | 0.00012756 | 123 | 41 | 3163 | 5.645 | 0.01343153 | 0.00673847 | 0.14304552 |
| PATHWAY | cell lineage | | | | | | | | | | |
| GOTERM_{—} | GO:0002822~regulation | 8 | 2.33 | 0.00025583 | 291 | 39 | 8731 | 6.155 | 0.42810616 | 0.00696069 | 0.44344237 |
| BP_ALL | of adaptive immune | | | | | | | | | | |
| | response based on | | | | | | | | | | |
| | somatic recombination | | | | | | | | | | |
| | of immune receptors | | | | | | | | | | |
| | built from | | | | | | | | | | |
| | immunoglobulin | | | | | | | | | | |
| | superfamily domains | | | | | | | | | | |
| GOTERM_{—} | GO:0051704~multi- | 30 | 8.72 | 0.00026472 | 291 | 436 | 8731 | 2.064 | 0.43910546 | 0.00711313 | 0.45881825 |
| BP_ALL | organism process | | | | | | | | | | |
| GOTERM_{—} | GO:0003008~system | 31 | 9.01 | 0.00029081 | 291 | 460 | 8731 | 2.022 | 0.47018397 | 0.00771672 | 0.50393593 |
| BP_ALL | process | | | | | | | | | | |
| GOTERM_{—} | GO:0050794~regulation | 168 | 48.84 | 0.00029607 | 291 | 4148 | 8731 | 1.215 | 0.47623627 | 0.00776147 | 0.51302706 |
| BP_ALL | of cellular process | | | | | | | | | | |
| GOTERM_{—} | GO:0002819~regulation | 8 | 2.33 | 0.00030141 | 291 | 40 | 8731 | 6.001 | 0.48230732 | 0.00780714 | 0.52225166 |
| BP_ALL | of adaptive | | | | | | | | | | |
| | immune response | | | | | | | | | | |
| GOTERM_{—} | GO:0031225~anchored | 11 | 3.20 | 0.00049512 | 305 | 89 | 9576 | 3.880 | 0.12602806 | 0.00789263 | 0.64978292 |
| CC_ALL | to membrane | | | | | | | | | | |
| GOTERM_{—} | GO:0042592~homeostatic | 29 | 8.43 | 0.00036654 | 291 | 423 | 8731 | 2.057 | 0.55097816 | 0.00937553 | 0.63477609 |
| BP_ALL | process | | | | | | | | | | |
| GOTERM_{—} | GO:0015718~monocarboxylic | 7 | 2.03 | 0.00038604 | 291 | 30 | 8731 | 7.001 | 0.56969876 | 0.00975755 | 0.66842859 |
| BP_ALL | acid transport | | | | | | | | | | |
| GOTERM_{—} | GO:0040012~regulation | 13 | 3.78 | 0.00041686 | 291 | 115 | 8731 | 3.392 | 0.59772396 | 0.01041227 | 0.72161898 |
| BP_ALL | of locomotion | | | | | | | | | | |
| GOTERM_{—} | GO:0006357~regulation | 32 | 9.30 | 0.00043124 | 291 | 493 | 8731 | 1.947 | 0.61016879 | 0.01064792 | 0.74642831 |
| BP_ALL | of transcription | | | | | | | | | | |
| | from RNA polymerase II | | | | | | | | | | |
| | promoter | | | | | | | | | | |
| GOTERM_{—} | GO:0031328~positive | 29 | 8.43 | 0.00050989 | 291 | 432 | 8731 | 2.014 | 0.67171554 | 0.01243745 | 0.88197876 |
| BP_ALL | regulation of cellular | | | | | | | | | | |
| | biosynthetic process | | | | | | | | | | |
| GOTERM_{—} | GO:0050878~regulation | 10 | 2.91 | 0.00053053 | 291 | 71 | 8731 | 4.226 | 0.68619313 | 0.01279497 | 0.91752731 |
| BP_ALL | of body fluid | | | | | | | | | | |
| GOTERM_{—} | GO:0010876~lipid | 12 | 3.49 | 0.00054417 | 291 | 102 | 8731 | 3.530 | 0.69540799 | 0.01297858 | 0.94101129 |
| BP_ALL | localization | | | | | | | | | | |
| GOTERM_{—} | GO:0050865~regulation | 12 | 3.49 | 0.00059161 | 291 | 103 | 8731 | 3.496 | 0.72540716 | 0.01395032 | 1.02266381 |
| BP_ALL | of cell activation | | | | | | | | | | |
| GOTERM_{—} | GO:0002237~response | 9 | 2.62 | 0.00060803 | 291 | 58 | 8731 | 4.656 | 0.73508127 | 0.01418162 | 1.05089343 |
| BP_ALL | to molecule of | | | | | | | | | | |
| | bacterial origin | | | | | | | | | | |
| GOTERM_{—} | GO:0030247~polysaccharide | 10 | 2.91 | 0.00014598 | 297 | 63 | 9375 | 5.010 | 0.08279861 | 0.01430145 | 0.21499395 |
| MF_ALL | binding | | | | | | | | | | |
| GOTERM_{—} | GO:0001871~pattern | 10 | 2.91 | 0.00014598 | 297 | 63 | 9375 | 5.010 | 0.08279861 | 0.01430145 | 0.21499395 |
| MF_ALL | binding | | | | | | | | | | |
| GOTERM_{—} | GO:0042127~regulation | 31 | 9.01 | 0.00063941 | 291 | 482 | 8731 | 1.930 | 0.75264313 | 0.01475101 | 1.10485745 |
| BP_ALL | of cell proliferation | | | | | | | | | | |
| GOTERM_{—} | GO:0009891~positive | 29 | 8.43 | 0.00066254 | 291 | 439 | 8731 | 1.982 | 0.76483615 | 0.01512107 | 1.14460859 |
| BP_ALL | regulation of | | | | | | | | | | |
| | biosynthetic | | | | | | | | | | |
| | process | | | | | | | | | | |
| GOTERM_{—} | GO:0044057~regulation | 14 | 4.07 | 0.00075021 | 291 | 140 | 8731 | 3.000 | 0.80584037 | 0.01692876 | 1.29513516 |
| BP_ALL | of system process | | | | | | | | | | |
| GOTERM_{—} | GO:0002697~regulation | 9 | 2.62 | 0.00076637 | 291 | 60 | 8731 | 4.501 | 0.81257953 | 0.01711373 | 1.32286309 |
| BP_ALL | of immune effector | | | | | | | | | | |
| GOTERM_{—} | GO:0046983~protein | 26 | 7.56 | 0.00020881 | 297 | 364 | 9375 | 2.255 | 0.11628999 | 0.01750586 | 0.30738349 |
| MF_ALL | dimerization activity | | | | | | | | | | |
| GOTERM_{—} | GO:0051270~regulation | 13 | 3.78 | 0.00088686 | 291 | 125 | 8731 | 3.120 | 0.8559733 | 0.01957882 | 1.52932924 |
| BP_ALL | of cell motion | | | | | | | | | | |
| GOTERM_{—} | GO:0007167~enzyme | 17 | 4.94 | 0.00094775 | 291 | 199 | 8731 | 2.563 | 0.87392438 | 0.02070065 | 1.63352589 |
| BP_ALL | linked receptor | | | | | | | | | | |
| | protein signaling | | | | | | | | | | |
| | pathway | | | | | | | | | | |
| GOTERM_{—} | GO:0009986~cell | 15 | 4.36 | 0.00141943 | 305 | 176 | 9576 | 2.676 | 0.3204743 | 0.02123574 | 1.85238613 |
| CC_ALL | surface | | | | | | | | | | |
| GOTERM_{—} | GO:0019725~cellular | 20 | 5.81 | 0.0009974 | 291 | 259 | 8731 | 2.317 | 0.88689134 | 0.02155829 | 1.71839807 |
| BP_ALL | homeostasis | | | | | | | | | | |
| GOTERM_{—} | GO:0009966~regulation | 35 | 10.17 | 0.00102968 | 291 | 588 | 8731 | 1.786 | 0.89459753 | 0.02203062 | 1.7735386 |
| BP_ALL | of signal transduction | | | | | | | | | | |
| GOTERM_{—} | GO:0032879~regulation | 24 | 6.98 | 0.00107922 | 291 | 344 | 8731 | 2.093 | 0.90541806 | 0.02242069 | 1.85812337 |
| BP_ALL | of localization | | | | | | | | | | |
| GOTERM_{—} | GO:0002683~negative | 8 | 2.33 | 0.001076 | 291 | 49 | 8731 | 4.899 | 0.90474892 | 0.02256898 | 1.85262047 |
| BP_ALL | regulation of | | | | | | | | | | |
| | immune system process | | | | | | | | | | |
| GOTERM_{—} | GO:0006869~lipid | 11 | 3.20 | 0.00107471 | 291 | 94 | 8731 | 3.511 | 0.90448101 | 0.0227608 | 1.85042799 |
| BP_ALL | transport | | | | | | | | | | |
| GOTERM_{—} | GO:0007399~nervous | 34 | 9.88 | 0.00117602 | 291 | 569 | 8731 | 1.793 | 0.92345953 | 0.02395313 | 2.02318447 |
| BP_ALL | system development | | | | | | | | | | |
| GOTERM_{—} | GO:0007154~cell | 24 | 6.98 | 0.00116651 | 291 | 346 | 8731 | 2.081 | 0.92185199 | 0.02398529 | 2.0069868 |
| BP_ALL | communication | | | | | | | | | | |
| GOTERM_{—} | GO:0001570~vasculogenesis | 6 | 1.74 | 0.00120076 | 291 | 25 | 8731 | 7.201 | 0.9274906 | 0.02422547 | 2.06533461 |
| BP_ALL | | | | | | | | | | | |
| GOTERM_{—} | GO:0008284~positive | 19 | 5.52 | 0.00126607 | 291 | 244 | 8731 | 2.136 | 0.93714055 | 0.02529364 | 2.17650973 |
| BP_ALL | regulation of | | | | | | | | | | |
| | cell proliferation | | | | | | | | | | |
| GOTERM_{—} | GO:0008285~negative | 18 | 5.23 | 0.00132516 | 291 | 225 | 8731 | 2.400 | 0.94475993 | 0.02575327 | 2.2769872 |
| BP_ALL | regulation of | | | | | | | | | | |
| | cell proliferation | | | | | | | | | | |
| GOTERM_{—} | GO:0042981~regulation | 35 | 10.17 | 0.00131483 | 291 | 596 | 8731 | 1.762 | 0.94349817 | 0.02578432 | 2.25943273 |
| BP_ALL | of apoptosis | | | | | | | | | | |
| GOTERM_{—} | GO:0002696~positive | 9 | 2.62 | 0.00131011 | 291 | 65 | 8731 | 4.154 | 0.94291175 | 0.02592551 | 2.25140603 |
| BP_ALL | regulation of | | | | | | | | | | |
| | leukocyte | | | | | | | | | | |
| | activation | | | | | | | | | | |
| GOTERM | GO:0002694~regulation | 11 | 3.20 | 0.00136994 | 291 | 97 | 8731 | 3.402 | 0.9499138 | 0.02637809 | 2.35308046 |
| BP_ALL | of leukocyte | | | | | | | | | | |
| | activation | | | | | | | | | | |
| GOTERM_{—} | GO:0008360~regulation | 7 | 2.03 | 0.00143618 | 291 | 38 | 8731 | 5.527 | 0.95666858 | 0.02739544 | 2.4655229 |
| BP_ALL | of cell shape | | | | | | | | | | |
| GOTERM_{—} | GO:0051173~positive | 27 | 7.85 | 0.00152867 | 291 | 419 | 8731 | 1.933 | 0.96460461 | 0.02863568 | 2.62231934 |
| BP_ALL | regulation of | | | | | | | | | | |
| | nitrogen compound | | | | | | | | | | |
| | metabolic process | | | | | | | | | | |
| GOTERM_{—} | GO:0043067~regulation | 35 | 10.17 | 0.00151875 | 291 | 602 | 8731 | 1.744 | 0.96382789 | 0.02869828 | 2.6055065 |
| BP_ALL | of programmed cell death | | | | | | | | | | |
| GOTERM_{—} | GO:0010941~regulation | 35 | 10.17 | 0.00162481 | 291 | 604 | 8731 | 1.739 | 0.97131776 | 0.03015225 | 2.78505643 |
| BP_ALL | of cell death | | | | | | | | | | |
| GOTERM_{—} | GO:0050727~regulation | 7 | 2.03 | 0.00165079 | 291 | 39 | 8731 | 5.385 | 0.97290246 | 0.03036957 | 2.82898994 |
| BP_ALL | of inflammatory | | | | | | | | | | |
| GOTERM_{—} | GO:0050930~induction | 4 | 1.16 | 0.00179385 | 291 | 8 | 8731 | 15.002 | 0.98018432 | 0.03268511 | 3.07055936 |
| BP_ALL | of positive chemotaxis | | | | | | | | | | |
| GOTERM_{—} | GO:0010646~regulation | 37 | 10.76 | 0.00194382 | 291 | 658 | 8731 | 1.687 | 0.98572763 | 0.03450973 | 3.32320019 |
| BP_ALL | of cell communication | | | | | | | | | | |
| GOTERM_{—} | GO:0050867~positive | 9 | 2.62 | 0.0019358 | 291 | 69 | 8731 | 3.913 | 0.98547495 | 0.03465108 | 3.30970571 |
| BP_ALL | regulation of | | | | | | | | | | |
| | cell activation | | | | | | | | | | |
| GOTERM_{—} | GO:0080134~regulation | 15 | 4.36 | 0.00192655 | 291 | 174 | 8731 | 2.587 | 0.98517793 | 0.03477294 | 3.29413758 |
| BP_ALL | of response to stress | | | | | | | | | | |
| GOTERM_{—} | GO:0001501~skeletal | 14 | 4.07 | 0.00202877 | 291 | 156 | 8731 | 2.693 | 0.98814896 | 0.03570233 | 3.46603944 |
| BP_ALL | system development | | | | | | | | | | |
| GOTERM_{—} | GO:0009615~response | 9 | 2.62 | 0.00212404 | 291 | 70 | 8731 | 3.858 | 0.99037937 | 0.03705101 | 3.62598828 |
| BP_ALL | to virus | | | | | | | | | | |
| GOTERM_{—} | GO:0009888~tissue | 22 | 6.40 | 0.00215578 | 291 | 319 | 8731 | 2.069 | 0.99102486 | 0.03729713 | 3.67920761 |
| BP_ALL | development | | | | | | | | | | |
| GOTERM_{—} | GO:0055066~di, | 12 | 3.49 | 0.00223579 | 291 | 121 | 8731 | 2.976 | 0.9924667 | 0.03835256 | 3.81327117 |
| BP_ALL | tri-valent inorganic | | | | | | | | | | |
| | cation homeostasis | | | | | | | | | | |
| GOTERM_{—} | GO:0022604~regulation | 10 | 2.91 | 0.00230132 | 291 | 87 | 8731 | 3.449 | 0.99347346 | 0.03914863 | 3.92295161 |
| BP_ALL | of cell morphogenesis | | | | | | | | | | |
| GOTERM_{—} | GO:0042493~response | 13 | 3.78 | 0.00236067 | 291 | 140 | 8731 | 2.786 | 0.99426863 | 0.03952428 | 4.02217244 |
| BP_ALL | to drug | | | | | | | | | | |
| GOTERM_{—} | GO:0035295~tube | 12 | 3.49 | 0.00238661 | 291 | 122 | 8731 | 2.951 | 0.99458499 | 0.03964677 | 4.06550921 |
| BP_ALL | development | | | | | | | | | | |
| GOTERM_{—} | GO:0043066~negative | 19 | 5.52 | 0.0023525 | 291 | 258 | 8731 | 2.210 | 0.99416515 | 0.03969391 | 4.0085168 |
| BP_ALL | regulation of apoptosis | | | | | | | | | | |
| GOTERM_{—} | GO:0005125~cytokine | 9 | 2.62 | 0.00055115 | 297 | 60 | 9375 | 4.735 | 0.27846423 | 0.03997568 | 0.80944805 |
| MF_ALL | activity | | | | | | | | | | |
| GOTERM_{—} | GO:0048146~positive | 5 | 1.45 | 0.00252304 | 291 | 18 | 8731 | 8.334 | 0.99598326 | 0.04155265 | 4.2931463 |
| BP_ALL | regulation of | | | | | | | | | | |
| GOTERM_{—} | GO:0044420~extracellular | 7 | 2.03 | 0.00313139 | 305 | 46 | 9576 | 4.778 | 0.57389694 | 0.04175683 | 4.04432272 |
| CC_ALL | matrix part | | | | | | | | | | |
| KEGG_{—} | hsa04610: Complement | 6 | 1.74 | 0.00122148 | 123 | 22 | 3163 | 7.013 | 0.12151451 | 0.04226609 | 1.36209977 |
| PATHWAY | and coagulation cascades | | | | | | | | | | |
| GOTERM_{—} | GO:0005604~basment | 6 | 1.74 | 0.00310557 | 305 | 32 | 9576 | 5.887 | 0.57088513 | 0.04355111 | 4.01160983 |
| CC_ALL | membrane | | | | | | | | | | |
| GOTERM_{—} | GO:0043069~negative | 19 | 5.52 | 0.00267451 | 291 | 261 | 8731 | 2.184 | 0.99711711 | 0.04366649 | 4.5452809 |
| BP_ALL | regulation of | | | | | | | | | | |
| | programmed cell death | | | | | | | | | | |
| GOTERM_{—} | GO:0060548~negative | 19 | 5.52 | 0.00277099 | 291 | 262 | 8731 | 2.176 | 0.99766616 | 0.04487296 | 4.70554415 |
| BP_ALL | regulation of cell death | | | | | | | | | | |
| GOTERM_{—} | GO:0050671~positive | 6 | 1.74 | 0.00281214 | 291 | 30 | 8731 | 6.001 | 0.9978673 | 0.04519043 | 4.77382529 |
| BP_ALL | regulation of lymphocyte | | | | | | | | | | |
| | proliferation | | | | | | | | | | |
| GOTERM_{—} | GO:0070665~positive | 6 | 1.74 | 0.00281214 | 291 | 30 | 8731 | 6.001 | 0.9978673 | 0.04519043 | 4.77382529 |
| BP_ALL | regulation of leukocyte | | | | | | | | | | |
| | proliferation | | | | | | | | | | |
| GOTERM_{—} | GO:0032946~positive | 6 | 1.74 | 0.00281214 | 291 | 30 | 8731 | 6.001 | 0.9978673 | 0.04519043 | 4.77382529 |
| BP_ALL | regulation of mononuclear | | | | | | | | | | |
| | cell proliferation | | | | | | | | | | |
| GOTERM_{—} | GO:0030141~secretory | 10 | 2.91 | 0.00359451 | 305 | 97 | 9576 | 3.237 | 0.62448874 | 0.04557029 | 4.62946073 |
| CC_ALL | granule | | | | | | | | | | |
| GOTERM_{—} | GO:0010628~positive | 24 | 6.98 | 0.00297411 | 291 | 372 | 8731 | 1.936 | 0.99850427 | 0.04704368 | 5.04213471 |
| BP_ALL | regulation of gene expression | | | | | | | | | | |
| GOTERM_{—} | GO:0010557~positive | 26 | 7.56 | 0.00295976 | 291 | 417 | 8731 | 1.871 | 0.99845649 | 0.04716276 | 5.01838762 |
| BP_ALL | regulation of macromolecule | | | | | | | | | | |
| | biosynthetic process | | | | | | | | | | |
| GOTERM_{—} | GO:0051241~negative | 9 | 2.62 | 0.00302532 | 291 | 74 | 8731 | 3.649 | 0.99866299 | 0.04749198 | 5.12681765 |
| BP_ALL | regulation of multicellular | | | | | | | | | | |
| | organismal | | | | | | | | | | |
| | process | | | | | | | | | | |
| GOTERM_{—} | GO:0007204~elevation | 7 | 2.03 | 0.00310921 | 291 | 44 | 8731 | 4.773 | 0.99888743 | 0.04843088 | 5.26537775 |
| BP_ALL | of cytosolic calcium ion | | | | | | | | | | |
| | concentration | | | | | | | | | | |
| GOTERM_{—} | GO:0001503~ossification | 8 | 2.33 | 0.00322266 | 291 | 59 | 8731 | 4.068 | 0.99913229 | 0.04980154 | 5.45247258 |
| BP_ALL | | | | | | | | | | | |

| Category | Genes |
|---|---|
| GOTERM_{—} | 210495_X_AT, 229221_AT, 223723_AT, 210136_AT, 212298_AT, 209555_S_AT, 212014_X_AT, |
| CC_ALL | 216834_AT, 203923_S_AT, 211066_X_AT, 210916_S_AT, 216971_S_AT, 219412_AT, 207674_AT, |
| | 202828_S_AT, 230360_AT, 236561_AT, 226545_AT, 201925_AT, 217173_S_AT, 204489_S_AT, |
| | 207610_S_AT, 220307_AT, 212099_AT, 211307_S_AT, 223303_AT, 201590_X_AT, 232549_AT, |
| | 203922_S_AT, 229937_X_AT, 214830_AT, 1552914_A_AT, 203104_AT, 212977_AT, 202756_S_AT, |
| | 228766_AT, 203508_AT, 230925_AT, 210845_S_AT, 243556_AT, 206033_S_AT, 201005_AT, |
| | 201242_S_AT, 233220_AT, 242907_AT, 207819_S_AT, 224678_AT, 209122_AT, 214211_AT, |
| | 217252_AT, 160020_AT, 208926_AT, 214297_AT, 210233_AT, 218910_AT, 211719_X_AT, 209949_AT, |
| | 214866_AT, 223501_AT, 205534_AT, 34210_AT, 205099_S_AT, 209994_S_AT, 1557905_S_AT, |
| | 228754_AT, 209670_AT, 238542_AT, 215836_S_AT, 217678_AT, 205717_X_AT, 217078_S_AT, |
| | 209047_AT, 238581_AT, 201739_AT, 1554992_AT, 209906_AT, 221840_AT, 211030_S_AT, 205920_AT, |
| | 208121_S_AT, 211676_S_AT, 1554600_S_AT, 238669_AT, 235295_AT, 203233_AT, 204105_S_AT, |
| | 201313_AT, 219994_AT, 201125_S_AT, 217279_X_AT, 201243_S_AT, 204912_AT, 201069_AT, |
| | 211924_S_AT, 222877_AT, 241938_AT, 203217_S_AT, 209079_X_AT, 206488_S_AT, 206171_AT, |
| | 220066_AT, 228640_AT, 203939_AT, 205542_AT, 243894_AT, 204359_AT, 213428_S_AT, 205891_AT, |
| | 204736_S_AT, 203665_AT, 204450_S_AT, 202748_AT, 208816_X_AT, 239519_AT, 209835_X_AT |
| | 208161_S_AT, 209921_AT, 204715_AT, 223502_S_AT, 212086_X_AT, 220484_AT, 203887_S_AT |
| | 223939_AT, 209146_AT, 202968_S_AT, 209392_AT, 241773_AT, 224859_AT, 209993_AT, 201324_AT, |
| | 201012_AT, 212464_S_AT, 216442_X_AT, 213503_X_AT, 227240_AT, 210427_X_AT, 214724_AT, |
| | 226218_AT, 204991_AT, 200748_S_AT, 227107_AT, 204401_AT, 211661_X_AT, 222062_AT, |
| | 202686_S_AT, 203548_S_AT, 223092_AT, 225188_AT, 217552_X_AT, 202827_S_AT, 205419_AT, |
| | 202071_AT, 210839_AT, 235299_AT, 224701_AT, 215332_AT, 202827_S_AT, 220313_AT, |
| | 205718_AT, 203888_AT, 212372_AT, 203935_AT, 223796_AT, 212049_AT, 212096_S_AT, 210258_AT, |
| | 1556583_A_AT, 229797_AT, 218223_S_AT, 226302_AT, 203411_S_AT, 241929_AT, 210173_AT, |
| | 202067_S_AT, 219434_AT, 215688_AT, 205098_AT |
| GOTERM_{—} | 201464_X_AT, 202540_S_AT, 203465_AT, 219028_AT, 212298_AT, 204972_AT, 212014_X_AT, 221731_X_AT, |
| BP_ALL | 216834_AT, 203923_S_AT, 207674_AT, 211068_AT, 205067_AT, 202859_X_AT, 201170_S_AT, 217173_S_AT, |
| | 204489_S_AT, 204620_S_AT, 211307_S_AT, 201147_S_AT, 201565_S_AT, 203922_S_AT, 2218715_AT, 1552914_A_AT, |
| | 213562_S_AT, 1555832_S_AT, 223876_AT, 2064495_S_AT, 208435_S_AT, 234211_AT, 216748_S_AT, 63825_AT, |
| | 205463_S_AT, 1405_I_AT, 201566_X_AT, 209949_AT, 211719_X_AT, 223501_AT, 229830_AT, 204058_AT, |
| | 202086_AT, 205093_S_AT, 1557905_S_AT, 235944_AT, 238542_AT, 209670_AT, 209047_AT, 217074_S_AT, |
| | 201150_S_AT, 2011 [symbol] _S_AT, 155 [symbol] _AT, 217757_AT, 20405 [symbol] _S_AT, 2132 [symbol] 1_AT, 201626_AT, 212 [symbol] _S_AT |
| | 23 [symbol] 669_AT, 2 [symbol] 223_AT, 3702 [symbol] _AT, 201243_S_AT, 217757_AT, 20 [symbol] _AT, 2 [symbol] 77_AT, 20270 [symbol] _S_AT, 201041_S_AT, |
| | 222 [symbol] 3_AT, 202434_S_AT, 212657_S_AT, 2064 [symbol] _S_AT, 220066_AT, 200878_AT, 203 [symbol] _AT, 205 [symbol] 1_AT, |
| | 23445 [symbol] _S_AT, 20274 [symbol] _AT, 23 [symbol] 19_AT, 20 [symbol] 35_X_AT, 225116_AT, 204715_AT, 22 [symbol] 502_S_AT, 201466_S_AT, |
| | 2 [symbol] 7_S_AT, 2029 [symbol] _S_AT, 20 [symbol] 37_S_AT, 20253 [symbol] _S_AT, 23621 [symbol] _AT, 21 [symbol] 4_S_AT, 2007 [symbol] _S_AT, 204401_AT, |
| | 202686_S_AT, 2220 [symbol] 2_AT, 210512_S_AT, 217552_X_AT, 211527_X_AT, 203140_AT, 220266_S_AT, 202241_AT, |
| | 202044_X_AT, 20 [symbol] 925_AT, 15565 [symbol] _A_AT, 229797_AT, 215 [symbol] _AT, 2010 [symbol] _AT, 229221_AT, 210 [symbol] 95_X_AT, |
| | 22013 [symbol] _AT, 209555_S_AT, 202435_S_AT, 2162 [symbol] 3_S_AT, 201473_AT, 203074_AT, 220916_S_AT, 201471_S_AT, |
| | 2 [symbol] 573 [symbol] _AT, 20 [symbol] 2_AT, 20282 [symbol] _S_AT, 236361_AT, 2022 [symbol] _S_AT, 20 [symbol] 27_S_AT, 201925_S_AT, 22 [symbol] 0_AT, |
| | 213577_AT, 229937_X_AT, 225115_AT, 20 [symbol] 50 [symbol] _AT, 210 [symbol] 45_S_AT, 203005_AT, 201242_S_AT, 20 [symbol] 1_S_AT, |
| | 233220_AT, 207 [symbol] 9_S_AT, 242907_AT, 209122_AT, 213293_S_AT, 1 [symbol] 0020_AT, 237252_AT, 212171_X_AT, [symbol] 02_AT |
| | 230233_AT, 2148 [symbol] _AT, 20 [symbol] 566_AT, 20 [symbol] 4_S_AT, 1552690_A_AT, 23 [symbol] 818_X_AT, 21767 [symbol] _AT, 2214 [symbol] 3_AT, |
| | 20 [symbol] 436_S_AT, 23 [symbol] _AT, 20 [symbol] 06_AT, 20172 [symbol] _AT, 20 [symbol] 21 [symbol] _AT, 20276 [symbol] _AT, 224606_AT, 210513_S_AT |
| | 215646_S_AT, 204619_S_AT, 233295_AT, 201313_AT, 215451_AT, 2250 [symbol] 7_AT, 217279_X_AT, 201627_S_AT, |
| | 20106 [symbol] _AT, 205552_S_AT, 221341_S_AT, 211524_S_AT, 212 [symbol] _AT, 213763_AT, 206171_AT, 202 [symbol] 14_AT, |
| | 204490_S_AT, 202436_S_AT, 20114 [symbol] _S_AT, 20 [symbol] 21_AT, 20 [symbol] 157_AT, 22 [symbol] 7_AT, 204 [symbol] _AT, 220 [symbol] 4_AT, |
| | 225368_AT, 20 [symbol] 2_AT, 22 [symbol] _AT, 201 [symbol] 25_S_AT, 20 [symbol] _AT, 201012_AT, 2124 [symbol] 4_S_AT, 216442_X_AT, |
| | 20 [symbol] 22_X_AT, 241722_X_AT, 227107_AT, 221 [symbol] 1_X_AT, 20 [symbol] 27_S_AT, 22 [symbol] 0 [symbol] 2_AT, 22 [symbol] 1 [symbol] _S_AT, [symbol] 71 [symbol] 0_AT, |
| | 205415_AT, 15 [symbol] 2467_AT, 210 [symbol] _S_AT, 20 [symbol] 65_S_AT, [symbol] 571_S_AT, 20 [symbol] 55_AT, 15 [symbol] 2553_A_AT, 20 [symbol] 1 [symbol] _AT, |
| | [symbol] 02437_S_AT, 2020 [symbol] _S_AT, 212372_AT, 20 [symbol] _AT, 227 [symbol] 7_AT, 217222_S_AT, [symbol] 56575 [symbol] _A_AT, 241 [symbol] 29_AT, |
| | 202067_S_AT, 215434_AT, [symbol] 11571_S_AT |
| GOTERM_{—} | 201464_X_AT, 229221_AT, 201465_S_AT, 210495_X_AT, 21229 [symbol] _AT, 209555_S_AT, 216243_S_AT, |
| BP_ALL | 212014_X_AT, 221731_X_AT, 201473_AT, 203923_S_AT, 203074_AT, 210916_S_AT, 235739_AT, |
| | 20 [symbol] 960_S_AT, 202828_S_AT, 205067_AT, 202859_X_AT, 202284_S_AT, 209827_S_AT, 201925_S_AT, |
| | 201170_S_AT, 204489_S_AT, 228490_AT, 204620_S_AT, 201147_S_AT, 203922_S_AT, 221815_AT, |
| | 1555832_S_AT, 228766_AT, 203508_AT, 210 [symbol] 45_S_AT, 201005_AT, 208961_S_AT, 233220_AT, |
| | 237252_AT, 160020_AT, 216248_S_AT, 63825_AT, 39402_AT, 205463_S_AT, 210233_AT, 1405_I_AT, |
| | 214 [symbol] 66_AT, 211719_X_AT, 229830_AT, 205099_S_AT, 205566_AT, 1557905_S_AT, 235944_AT, |
| | 221463_AT, 208436_S_AT, 201150_S_AT, 201148_S_AT, 209906_AT, 217757_AT, 213281_AT, |
| | 224606_AT, 204619_S_AT, 215646_S_AT, 217279_X_AT, 37152_AT, 221841_S_AT, 211924_S_AT, |
| | 222877_AT, 201041_S_AT, 212657_S_AT, 206488_S_AT, 206171_AT, 220066_AT, 205891_AT, |
| | 203665_AT, 204490_S_AT, 201149_S_AT, 239519_AT, 209835_X_AT, 206157_AT, 201466_S_AT, |
| | 225337_AT, 204035_AT, 203887_S_AT, 209392_AT, 201012_AT, 212464_S_AT, 216442_X_AT, |
| | 204622_X_AT, 211661_X_AT, 202686_S_AT, 217552_X_AT, 202827_S_AT, 87100_AT, 210839_S_AT, |
| | 204465_S_AT, 220266_S_AT, 204655_AT, 1552553_A_AT, 209189_AT, 201044_X_AT, 203888_AT, |
| | 212372_AT, 203935_AT, 1556583_A_AT, 227697_AT, 1555759_A_AT, 241929_AT, 211571_S_AT, |
| | 205098_AT |
| GOTERM_{—} | 229221_AT, 210495_X_AT, 37152_AT, 212298_AT, 209555_S_AT, 216243_S_AT, 211924_S_AT, |
| BP_ALL | 212014_X_AT, 221731_X_AT, 203923_S_AT, 203074_AT, 210016_S_AT, 212657_S_AT, 206488_S_AT, |
| | 20 [symbol] 960_S_AT, 206171_AT, 205067_AT, 202859_X_AT, 201925_S_AT, 204489_S_AT, 22 [symbol] 490_AT, |
| | 203665_AT, 204490_S_AT, 204620_S_AT, 201149_S_AT, 201147_S_AT, 239519_AT, 209835_X_AT, |
| | 203922_S_AT, 206157_AT, 221815_AT, 225337_AT, 204035_AT, 203887_S_AT, 1555832_S_AT, |
| | 228766_AT, 203508_AT, 210845_S_AT, 201005_AT, 208961_S_AT, 201012_AT, 212464_S_AT, |
| | 237252_AT, 216442_X_AT, 39402_AT, 63825_AT, 205463_S_AT, 210233_AT, 1405_I_AT, 214866_AT, |
| | 211719_X_AT, 229830_AT, 205099_S_AT, 211661_X_AT, 205566_AT, 1557905_S_AT, 235944_AT, |
| | 217552_X_AT, 208436_S_AT, 221463_AT, 87100_AT, 201150_S_AT, 204465_S_AT, 201148_S_AT, |
| | 209906_AT, 217757_AT, 224606_AT, 204655_AT, 1552553_A_AT, 209159_AT, 215646_S_AT, |
| | 204619_S_AT, 203888_AT, 212372_AT, 203935_AT, 1555759_A_AT, 241929_AT, 211571_S_AT, |
| | 205098_AT |
| GOTERM_{—} | 223723_AT, 229221_AT, 209555_S_AT, 212014_X_AT, 203923_S_AT, 205217_S_AT, 210916_S_AT, |
| CC_ALL | 207674_AT, 206488_S_AT, 206171_AT, 202828_S_AT, 236561_AT, 228640_AT, 201925_S_AT, |
| | 205542_AT, 217173_S_AT, 204489_S_AT, 204359_AT, 204736_S_AT, 205 [symbol] 91_AT, 204490_S_AT, |
| | 211307_S_AT, 209 [symbol] 35_X_AT, 208161_S_AT, 203922_S_AT, 203104_AT, 202756_S_AT, 203887_S_AT, |
| | 228766_AT, 241773_AT, 209392_AT, 201005_AT, 201242_S_AT, 233230_AT, 207819_S_AT, |
| | 160020_AT, 237252_AT, 214297_AT, 210333_AT, 22 [symbol] 910_AT, 209949_AT, 205534_AT, 204661_AT, |
| | 34210_AT, 205099_S_AT, 204401_AT, 211661_X_AT, 1557905_S_AT, 202 [symbol] 86_S_AT, 222062_AT, |
| | 22 [symbol] 754_AT, 209670_AT, 202827_S_AT, 217552_X_AT, 22 [symbol] 092_AT, 209047_AT, 205419_AT, |
| | 202071_AT, 210839_S_AT, 209906_AT, 202068_S_AT, 205 [symbol] 20_AT, 211030_S_AT, 211676_S_AT, |
| | 208121_S_AT, 205718_AT, 203888_AT, 203935_AT, 203253_AT, 204105_S_AT, 1556583_A_AT, |
| | 225302_AT, 210173_AT, 241929_AT, 217279_X_AT, 201125_S_AT, 202067_S_AT, 201243_S_AT, |
| | 205098_AT |
| GOTERM_{—} | 223723_AT, 229221_AT, 209555_S_AT, 212014_X_AT, 203923_S_AT, 203217_S_AT, 210916_S_AT, |
| CC_ALL | 207674_AT, 206488_S_AT, 206171_AT, 202828_S_AT, 236561_AT, 228640_AT, 201925_S_AT, |
| | 205542_AT, 217173_S_AT, 204489_S_AT, 204359_AT, 204736_S_AT, 205891_AT, 204490_S_AT, |
| | 211307_S_AT, 209835_X_AT, 208161_S_AT, 203922_S_AT, 203104_AT, 202756_S_AT, 203887_S_AT, |
| | 228766_AT, 241773_AT, 209392_AT, 201005_AT, 201242_S_AT, 233220_AT, 207819_S_AT, |
| | 160020_AT, 237252_AT, 214297_AT, 210233_AT, 228910_AT, 209949_AT, 205534_AT, 204661_AT, |
| | 34210_AT, 205099_S_AT, 204401_AT, 211661_X_AT, 1557905_S_AT, 202686_S_AT, 222062_AT, |
| | 228754_AT, 209670_AT, 202827_S_AT, 217552_X_AT, 223092_AT, 209047_AT, 205419_AT, |
| | 202071_AT, 210839_S_AT, 209906_AT, 202068_S_AT, 205920_AT, 211030_S_AT, 211676_S_AT, |
| | 208121_S_AT, 205718_AT, 203888_AT, 203935_AT, 203233_AT, 204105_S_AT, 1556583_A_AT, |
| | 226302_AT, 210173_AT, 241929_AT, 217279_X_AT, 201125_S_AT, 202067_S_AT, 201243_S_AT, |
| | 205098_AT |
| GOTERM_{—} | 229221_AT, 223723_AT, 210495_X_AT, 212298_AT, 216243_S_AT, 212014_X_AT, 221731_X_AT, |
| CC_ALL | 210916_S_AT, 207674_AT, 202828_S_AT, 205067_AT, 228648_AT, 202859_X_AT, 226545_AT, |
| | 209827_S_AT, 217173_S_AT, 204489_S_AT, 204620_S_AT, 211307_S_AT, 201147_S_AT, 201590_X_AT, |
| | 202756_S_AT, 205495_S_AT, 204834_AT, 203508_AT, 228873_AT, 210845_S_AT, 210095_S_AT, |
| | 209122_AT, 237252_AT, 160020_AT, 212171_X_AT, 39402_AT, 205463_S_AT, 210233_AT, 1405_I_AT, |
| | 211719_X_AT, 214866_AT, 223501_AT, 229830_AT, 1557905_S_AT, 206835_AT, 238542_AT, |
| | 235944_AT, 221463_AT, 201150_S_AT, 201148_S_AT, 217757_AT, 229004_AT, 204619_S_AT, |
| | 215646_S_AT, 210513_S_AT, 203233_AT, 202087_S_AT, 216546_S_AT, 217279_X_AT, 201069_AT, |
| | 211924_S_AT, 204222_S_AT, 212657_S_AT, 203665_AT, 213428_S_AT, 204359_AT, 204490_S_AT, |
| | 208816_X_AT, 201149_S_AT, 239519_AT, 209835_X_AT, 212190_AT, 206157_AT, 223502_S_AT, |
| | 212143_S_AT, 202458_AT, 204035_AT, 225955_AT, 203887_S_AT, 228762_AT, 209392_AT, |
| | 212464_S_AT, 216442_X_AT, 213503_X_AT, 210427_X_AT, 226218_AT, 203940_S_AT, 227265_AT, |
| | 219908_AT, 203548_S_AT, 210512_S_AT, 202827_S_AT, 225186_S_AT, 211527_X_AT, 204475_AT, |
| | 210839_S_AT, 204655_AT, 202068_S_AT, 203888_AT, 1555759_A_AT, 219434_AT, 202067_S_AT, |
| | 211571_S_AT |
| GOTERM_{—} | 229223_AT, 210495_X_AT, 201069_AT, 216243_S_AT, 212014_X_AT, 221731_X_AT, 210916_S_AT, |
| CC_ALL | 212657_S_AT, 202828_S_AT, 205067_AT, 202859_X_AT, 226545_AT, 209827_S_AT, 217173_S_AT, |
| | 204489_S_AT, 203665_AT, 204359_AT, 213428_S_AT, 204490_S_AT, 208816_X_AT, 204620_S_AT, |
| | 201149_S_AT, 201147_S_AT, 209835_X_AT, 201590_X_AT, 212190_AT, 223502_S_AT, 212143_S_AT, |
| | 204035_AT, 202756_S_AT, 203887_S_AT, 205495_S_AT, 204834_AT, 228873_AT, 210095_S_AT, |
| | 212464_S_AT, 160020_AT, 237252_AT, 216442_X_AT, 212171_X_AT, 213503_X_AT, 39402_AT, |
| | 205463_S_AT, 1405_I_AT, 210427_X_AT, 211719_X_AT, 223501_AT, 229830_AT, 1557905_S_AT, |
| | 203940_S_AT, 227265_AT, 238542_AT, 203548_S_AT, 235944_AT, 219908_AT, 210512_S_AT, |
| | 202827_S_AT, 221463_AT, 211527_X_AT, 204475_AT, 202150_S_AT, 201148_S_AT, 217757_AT, |
| | 229004_AT, 204655_AT, 210513_S_AT, 215646_S_AT, 204619_S_AT, 202068_S_AT, 203888_AT, |
| | 1555759_A_AT, 216546_S_AT, 217279_X_AT, 202067_S_AT, 211571_S_AT |
| GOTERM_{—} | 201464_X_AT, 201465_S_AT, 210495_X_AT, 37152_AT, 212298_AT, 210136_AT, 209555_S_AT, |
| BP_ALL | 227948_AT, 221841_S_AT, 201473_AT, 204923_AT, 206488_S_AT, 1566901_AT, 214255_AT, |
| | 205067_AT, 236561_AT, 1569149_AT, 216017_S_AT, 203665_AT, 205891_AT, 204998_S_AT, |
| | 212099_AT, 201565_S_AT, 239519_AT, 201466_S_AT, 1552914_A_AT, 212143_S_AT, 215602_AT, |
| | 228766_AT, 208937_S_AT, 205312_AT, 224859_AT, 210095_S_AT, 212464_S_AT, 216442_X_AT, |
| | 212171_X_AT, 39402_AT, 1405_I_AT, 201566_X_AT, 211719_X_AT, 226218_AT, 202191_S_AT, |
| | 218559_S_AT, 206835_AT, 222062_AT, 203940_S_AT, 200921_S_AT, 203548_S_AT, 210512_S_AT, |
| | 223092_AT, 222670_S_AT, 200920_S_AT, 211527_X_AT, 203751_X_AT, 203140_AT, 220266_S_AT, |
| | 220935_S_AT, 213281_AT, 204655_AT, 210513_S_AT, 1564754_X_AT, 212372_AT, 203935_AT, |
| | 1565752_AT, 203233_AT, 204105_S_AT, 227697_AT, 203752_S_AT, 219257_S_AT, 212803_AT, |
| | 1555759_A_AT, 241929_AT |
| GOTERM_{—} | 201464_X_AT, 201465_S_AT, 210136_AT, 212298_AT, 204923_AT, 235739_AT, 240665_AT, |
| BP_ALL | 206171_AT, 1566901_AT, 205067_AT, 236561_AT, 220066_AT, 200878_AT, 201170_S_AT, |
| | 1569149_AT, 216017_S_AT, 205891_AT, 203665_AT, 204998_S_AT, 208816_X_AT, 212099_AT, |
| | 201565_S_AT, 239519_AT, 201590_X_AT, 204715_AT, 201466_S_AT, 1552914_A_AT, 212143_S_AT, |
| | 205312_AT, 208937_S_AT, 224859_AT, 210095_S_AT, 216248_S_AT, 213503_X_AT, 212171_X_AT, |
| | 39402_AT, 204622_X_AT, 205463_S_AT, 1405_I_AT, 201566_X_AT, 210427_X_AT, 226218_AT, |
| | 218934_S_AT, 229830_AT, 218559_S_AT, 227107_AT, 206835_AT, 222062_AT, 203940_S_AT, |
| | 200921_S_AT, 222670_S_AT, 210512_S_AT, 223092_AT, 200920_S_AT, 202071_AT, 211527_X_AT, |
| | 1562467_AT, 203751_X_AT, 1554992_AT, 203140_AT, 213281_AT, 220935_S_AT, 204655_AT, |
| | 210513_S_AT, 238669_AT, 235295_AT, 203935_AT, 203233_AT, 204105_S_AT, 1556583_A_AT, |
| | 203752_S_AT, 219257_S_AT, 212803_AT, 1555759_A_AT, 215688_AT |
| GOTERM_{—} | 204912_AT, 229221_AT, 37152_AT, 212298_AT, 209555_S_AT, 216243_S_AT, 211924_S_AT, |
| MF_ALL | 212014_X_AT, 222877_AT, 210916_S_AT, 211352_S_AT, 235739_AT, 212657_S_AT, 207674_AT, |
| | 206488_S_AT, 206171_AT, 242871_AT, 236561_AT, 217173_S_AT, 204489_S_AT, 205891_AT, |
| | 204490_S_AT, 207610_S_AT, 220307_AT, 211307_S_AT, 239519_AT, 209835_X_AT, 218856_AT, |
| | 21 [symbol] 686_S_AT, 229937_X_AT, 207700_S_AT, 203104_AT, 212977_AT, 223939_AT, 203887_S_AT, |
| | 228766_AT, 203508_AT, 210845_S_AT, 241773_AT, 209392_AT, 233220_AT, 204774_AT, |
| | 214581_X_AT, 237252_AT, 216248_S_AT, 204622_X_AT, 210233_AT, 214866_AT, 226218_AT, |
| | 205099_S_AT, 211661_X_AT, 1557905_S_AT, 2026 [symbol] 6_S_AT, 222062_AT, 209670_AT, 235944_AT, |
| | 238542_AT, 217552_X_AT, 217078_S_AT, 205419_AT, 202071_AT, 210839_S_AT, 209906_AT, |
| | 221840_AT, 202068_S_AT, 211676_S_AT, 208121_S_AT, 220313_AT, 205718_AT, 203888_AT, |
| | 203935_AT, 203233_AT, 210173_AT, 241929_AT, 201125_S_AT, 202067_S_AT, 219434_AT, 2050 [symbol] 8_AT |
| GOTERM_{—} | 229221_AT, 21229 [symbol] _AT, 209555_S_AT, 216243_S_AT, 212014_X_AT, 216834_AT, 210916_S_AT, |
| MF_ALL | 211352_S_AT, 235739_AT, 207674_AT, 242871_AT, 236561_AT, 217173_S_AT, 2044 [symbol] _S_AT, |
| | 203760_S_AT, 207610_S_AT, 220307_AT, 211307_S_AT, 229937_X_AT, 218686_S_AT, 207700_S_AT, |
| | 20 [symbol] 104_AT, 212977_AT, 228766_AT, 203508_AT, 210845_S_AT, 233220_AT, 237252_AT, |
| | 214581_X_AT, 214297_AT, 216248_S_AT, 220233_AT, 1405_I_AT, 214866_AT, 205099_S_AT, |
| | 1557905_S_AT, 235 [symbol] 44_AT, 238542_AT, 209670_AT, 217078_S_AT, 209906_AT, 221 [symbol] 40_AT, |
| | 20 [symbol] 121_S_AT, 211676_S_AT, 203233_AT, 217591_AT, 201125_S_AT, 204912_AT, 37152_AT, |
| | 211924_S_AT, 222877_AT, 212657_S_AT, 2064 [symbol] _S_AT, 206171_AT, 200878_AT, 202388_AT, |
| | 204736_S_AT, 205891_AT, 203665_AT, 204359_AT, 204490_S_AT, 239519_AT, 20 [symbol] 35_X_AT, |
| | 218856_AT, 203887_S_AT, 223939_AT, 202988_S_AT, 209392_AT, 241773_AT, 204774_AT, |
| | 204622_X_AT, 214724_AT, 226218_AT, 211661_X_AT, 222062_AT, 2026 [symbol] 6_S_AT, 225207_AT, |
| | 217552_AT, 205419_AT, 202071_AT, 210839_S_AT, 204655_AT, 202068_S_AT, 214054_AT, |
| | 220313_AT, 205718_AT, 201 [symbol] 88_AT, 203935_AT, 203761_AT, 210258_AT, 1555759_A_AT, 210173_AT, |
| | 241929_AT, 21 [symbol] 34_AT, 202067_S_AT, 203320_AT, 2050 [symbol] _AT |
| GOTERM_{—} | 229221_AT, 212298_AT, 209555_S_AT, 216243_S_AT, 212014_X_AT, 216834_AT, 210916_S_AT, |
| MF_ALL | 211352_S_AT, 23573 [symbol] _AT, 207574_AT, 242871_AT, 232563_AT, 217173_S_AT, 2044 [symbol] 9_S_AT, |
| | 203760_S_AT, 207610_S_AT, 220307_AT, 211307_S_AT, 229937_X_AT, 21 [symbol] 6_S_AT, 207700_S_AT, |
| | 203104_AT, 212977_AT, 228766_AT, 20 [symbol] 50 [symbol] _AT, 210845_S_AT, 233220_AT, 237252_AT, |
| | 214581_X_AT, 214297_AT, 216248_S_AT, 210233_AT, 1405_I_AT, 214 [symbol] 6_AT, 205099_S_AT |
| | 1557905_S_AT, 235944_AT, 23 [symbol] 542_AT, 209670_AT, 217078_AT, 209906_AT, 221 [symbol] 40_AT, |
| | 2 [symbol] 121_S_AT, 211676_S_AT, 203233_AT, 2175 [symbol] 1_AT, 201125_S_AT, 204912_AT, 37152_AT, |
| | 211924_S_AT, 222877_AT, 212657_S_AT, 2064 [symbol] _S_AT, 206171_AT, 200 [symbol] 7 [symbol] _AT, 202388_AT, |
| | 204736_S_AT, 20 [symbol] 1_AT, 203665_AT, 204359_AT, 204490_S_AT, 239519_AT, 209835_X_AT, |
| | 218856_AT, 203 [symbol] 7_S_AT, 223939_AT, 20298 [symbol] _S_AT, 209392_AT, 241773_AT, 204774_AT, |
| | 204622_X_AT, 214724_AT, 226214_AT, 211661_X_AT, 222062_AT, 202686_S_AT, 225207_AT, |
| | 217552_X_AT, 205419_AT, 202071_AT, 210 [symbol] 39_S_AT, 204655_AT, 202068_S_AT, 214054_AT, |
| | 220313_AT, 20571 [symbol] _AT, 203888_AT, 203935_AT, 20 [symbol] 761_AT, 210258_AT, 155575 [symbol] _A_AT, 210173_AT, |
| | 241 [symbol] 29_AT, 219434_AT, 202067_S_AT, 203320_AT, 20509 [symbol] _AT |
| GOTERM_{—} | 237252_AT, 204912_AT, 21229 [symbol] _AT, 20 [symbol] 555_S_AT, 210233_AT, 222877_AT, 22621 [symbol] _AT, |
| MF_ALL | 205099_S_AT, 211661_X_AT, 206488_S_AT, 222062_AT, 202686_S_AT, 206171_AT, 238542_AT, |
| | 235944_AT, 236561_AT, 217552_X_AT, 205419_AT, 210 [symbol] 39_S_AT, 217173_S_AT, 20 [symbol] 906_AT, |
| | 205 [symbol] 1_AT, 207610_S_AT, 221840_AT, 239519_AT, 202068_S_AT, 20 [symbol] 121_S_AT, 211676_S_AT, |
| | 220313_AT, 203888_AT, 203935_AT, 203104_AT, 203233_AT, 212977_AT, 203887_S_AT, 223939_AT, |
| | 228766_AT, 203508_AT, 20 [symbol] 92_AT, 24192 [symbol] _AT, 241773_AT, 210173_AT, 202067_S_AT, 233220_AT, |
| | 204774_AT, 2050 [symbol] 8_AT |
| GOTERM_{—} | 201464_X_AT, 160020_AT, 201465_S_AT, 229221_AT, 201069_AT, 212171_X_AT, 213503_X_AT, |
| BP_ALL | 21229 [symbol] _AT, 214297_AT, 39402_AT, 205463_S_AT, 212014_X_AT, 210427_X_AT, 201473_AT, |
| | 222 [symbol] 77_AT, 210916_S_AT, 24193 [symbol] _AT, 22 [symbol] 30_AT, 1557905_S_AT, 211962_S_AT, 202 [symbol] 2 [symbol] _S_AT, |
| | 205067_AT, 210512_S_AT, 202827_S_AT, 202 [symbol] 59_X_AT, 236561_AT, 211527_X_AT, 200 [symbol] 78_AT, |
| | 204489_S_AT, 20 [symbol] 5_AT, 204736_S_AT, 204490_S_AT, 2132 [symbol] 1_AT, 212099_AT, 208816_X_AT, |
| | 310513_S_AT, 239519_AT, 209 [symbol] 35_X_AT, 201590_X_AT, 201466_S_AT, 203955_AT, 204035_AT, |
| | 212124_AT, 20 [symbol] 37_S_AT, 210173_AT, 2172 [symbol] _X_AT |
| GOTERM_{—} | 229221_AT, 223723_AT, 210495_X_AT, 210136_AT, 209555_S_AT, 212014_X_AT, 203923_S_AT, |
| CC_ALL | 210915_S_AT, 219412_AT, 207674_AT, 202828_S_AT, 236561_AT, 201925_S_AT, 217173_S_AT, |
| | 204489_S_AT, 220 [symbol] 07_AT, 211307_S_AT, 223303_AT, 203922_S_AT, 214 [symbol] 30_AT, 1552914_A_AT, |
| | 203304_AT, 202756_S_AT, 228766_AT, 230 [symbol] 25_AT, 201005_AT, 206033_S_AT, 201242_S_AT, |
| | 233220_AT, 242907_AT, 207819_S_AT, 237252_AT, 160020_AT, 214297_AT, 210233_AT, 228910_AT, |
| | 209949_AT, 211719_X_AT, 34210_AT, 205534_AT, 2050 [symbol] _S_AT, 20 [symbol] 94_S_AT, 1557905_S_AT, |
| | 22 [symbol] 754_AT, 23 [symbol] 542_AT, 209670_AT, 209047_AT, 238581_AT, 209906_AT, 211030_S_AT, 205520_AT, |
| | 20 [symbol] 121_S_AT, 211676_S_AT, 2352 [symbol] 5_AT, 204105_S_AT, 20 [symbol] 233_AT, 219994_AT, 201125_S_AT, |
| | 217279_X_AT, 201243_S_AT, 203217_S_AT, 2064 [symbol] _S_AT, 206171_AT, 22 [symbol] 640_AT, 205542_AT, |
| | 204736_S_AT, 205891_AT, 203665_AT, 20435 [symbol] _AT, 204490_S_AT, 202748_AT, 209835_X_AT, |
| | 20 [symbol] 161_S_AT, 204715_AT, 203887_S_AT, 209392_AT, 241773_AT, 224859_AT, 209999_AT, |
| | 201012_AT, 212464_S_AT, 216442_X_AT, 214724_AT, 226218_AT, 204661_AT, 227107_AT, |
| | 204401_AT, 211661_X_AT, 222062_AT, 202686_S_AT, 217552_X_AT, 225188_AT, 2230 [symbol] 2_AT, |
| | 202 [symbol] 27_S_AT, 205419_AT, 202071_AT, 210839_S_AT, 219332_AT, 20206 [symbol] _S_AT, 20671 [symbol] _AT, |
| | 203888_AT, 203935_AT, 15565 [symbol] 3_A_AT, 22 [symbol] 302_AT, 241929_AT, 210173_AT, 202067_S_AT, |
| | 205098_AT |
| GOTERM_{—} | 201464_X_AT, 202540_S_AT, 227069_AT, 229221_AT, 201465_S_AT, 210136_AT, 21229 [symbol] _AT, |
| BP_ALL | 212014_X_AT, 221731_X_AT, 201473_AT, 210 [symbol] 16_S_AT, 235739_AT, 206472_S_AT, 20 [symbol] 960_S_AT, |
| | 202828_S_AT, 205067_AT, 209 [symbol] 03_S_AT, 23 [symbol] 561_AT, 230360_AT, 202 [symbol] 59_X_AT, 2022 [symbol] 4_S_AT, |
| | 1569149_AT, 2044 [symbol] 9_S_AT, 216017_S_AT, 204620_S_AT, 2120 [symbol] 9_AT, 201147_S_AT, 201565_S_AT, |
| | 201590_X_AT, 232649_AT, 204653_AT, 1555832_S_AT, 210 [symbol] 45_S_AT, 243556_AT, 201005_AT, |
| | 20 [symbol] 61_S_AT, 233220_AT, 210095_S_AT, 160020_AT, 234967_AT, 216248_S_AT, 214297_AT, |
| | 212171_X_AT, 39402_AT, 205463_S_AT, 201566_X_AT, 214866_AT, 22 [symbol] 964_AT, 202191_S_AT, |
| | 229 [symbol] 30_AT, 1557905_S_AT, 21 [symbol] 502_S_AT, 20 [symbol] 35_AT, 222670_S_AT, 201150_S_AT, 203751_X_AT, |
| | 20114 [symbol] _S_AT, 1554992_AT, 213283_AT, 224606_AT, 204619_S_AT, 215646_S_AT, 210513_S_AT, |
| | 1554600_S_AT, 204105_S_AT, 204 [symbol] 1_S_AT, 203752_S_AT, 212 [symbol] 03_AT, 225557_AT, 217279_X_AT, |
| | 37152_AT, 20106 [symbol] _AT, 221 [symbol] 41_S_AT, 211924_S_AT, 22421 [symbol] _S_AT, 222 [symbol] 77_AT, 241938_AT, |
| | 211962_S_AT, 15 [symbol] 6901_AT, 200 [symbol] 78_AT, 204141_AT, 204736_S_AT, 203665_AT, 2044 [symbol] 0_S_AT, |
| | 204998_S_AT, 20 [symbol] 816_X_AT, 201149_S_AT, 23 [symbol] 519_AT, 209835_X_AT, 212190_AT, 201466_S_AT, |
| | 212143_S_AT, 204035_AT, 212086_X_AT, 220484_AT, 212124_AT, 20 [symbol] 37_S_AT, 22 [symbol] 762_AT, |
| | 222651_S_AT, 2119 [symbol] 6_AT, 230529_AT, 201324_AT, 201012_AT, 202539_S_AT, 213503_X_AT, |
| | 204622_X_AT, 227240_AT, 210427_X_AT, 226218_AT, 21 [symbol] 559_S_AT, 202686_S_AT, 204 [symbol] 4_AT, |
| | 219845_AT, 210512_S_AT, 223092_AT, 202 [symbol] 27_S_AT, 205419_AT, 211527_X_AT, 1562467_AT, |
| | 2 [symbol] 4465_S_AT, 203140_AT, 220266_S_AT, 220935_S_AT, 2091 [symbol] 9_AT, 212372_AT, 203935_AT, |
| | 2120 [symbol] 9_AT, 2019 [symbol] 5_AT, 227697_AT, 229797_AT, 203411_S_AT, 210173_AT, 211571_S_AT, |
| | 2156 [symbol] _AT, 203320_AT |
| GOTERM_{—} | 201464_X_AT, 160020_AT, 2014 [symbol] 5_S_AT, 214297_AT, 21229 [symbol] _AT, 213503_X_AT, 212171_X_AT, |
| BP_ALL | 39402_AT, 205463_S_AT, 210427_X_AT, 201473_AT, 222877_AT, 24193 [symbol] _AT, 229 [symbol] 30_AT, |
| | 211962_S_AT, 202828_S_AT, 205067_AT, 210512_S_AT, 202 [symbol] 5 [symbol] _X_AT, 202 [symbol] 27_S_AT, 236561_AT, |
| | 211527_X_AT, 200 [symbol] 78_AT, 20366 [symbol] _AT, 204736_S_AT, 2132 [symbol] 1_AT, 2120 [symbol] 9_AT, 20 [symbol] 816_X_AT, |
| | 23 [symbol] 519_AT, 210513_S_AT, 201590_X_AT, 201466_S_AT, 203935_AT, 204035_AT, 212124_AT, |
| | 20 [symbol] 37_S_AT, 210173_AT, 21727 [symbol] _X_AT |
| GOTERM_{—} | 201464_X_AT, 160020_AT, 201465_S_AT, 229221_AT, 201069_AT, 212171_X_AT, 213503_X_AT, |
| BP_ALL | 212298_AT, 214297_AT, 39402_AT, 205463_S_AT, 212014_X_AT, 210427_X_AT, 201473_AT |
| | 222 [symbol] 77_AT, 210916_S_AT, 241938_AT, 229 [symbol] 30_AT, 1557905_S_AT, 211962_S_AT, 202828_S_AT, |
| | 205067_AT, 210512_S_AT, 202827_S_AT, 20285 [symbol] _X_AT, 236561_AT, 211527_X_AT, 200 [symbol] 7 [symbol] _AT, |
| | 204489_S_AT, 203665_AT, 204736_S_AT, 204490_S_AT, 213281_AT, 212099_AT, 208816_X_AT |
| | 210513_S_AT, 239519_AT, 209635_X_AT, 201590_X_AT, 201466_S_AT, 203935_AT, 204035_AT, |
| | 212124_AT, 208937_S_AT, 210173_AT, 217279_X_AT |
| GOTERM_{—} | 201464_X_AT, 210495_X_AT, 229221_AT, 201465_S_AT, 219028_AT, 21229 [symbol] _AT, 209555_S_AT, |
| BP_ALL | 216243_S_AT, 212014_X_AT, 221731_X_AT, 203923_S_AT, 203074_AT, 210916_S_AT, 201471_S_AT |
| | 235739_AT, 20 [symbol] 960_S_AT, 202 [symbol] 28_S_AT, 205067_AT, 236561_AT, 202859_X_AT, 202284_S_AT, |
| | 201925_S_AT, 228490_AT, 204489_S_AT, 204620_S_AT, 201147_S_AT, 203922_S_AT, 221 [symbol] 15_AT, |
| | 225115_AT, 1555832_S_AT, 228766_AT, 205495_S_AT, 203508_AT, 210 [symbol] 45_S_AT, 201005_AT, |
| | 208961_S_AT, 201242_S_AT, 20 [symbol] 43 [symbol] _S_AT, 237252_AT, 160020_AT, 212171_X_AT, 21624 [symbol] _S_AT, |
| | 63825_AT, 39402_AT, 205463_S_AT, 210233_AT, 1405_I_AT, 214866_AT, 211719_X_AT, 209949_AT, |
| | 229 [symbol] 30_AT, 202086_AT, 20509 [symbol] _S_AT, 205566_AT, 1557 [symbol] 05_S_AT, 23 [symbol] 18_X_AT, 209670_AT, |
| | 235944_AT, 221463_AT, 20 [symbol] 436_S_AT, 201150_S_AT, 20114 [symbol] _S_AT, 20 [symbol] 06_AT, 201739_AT, |
| | 217757_AT, 213281_AT, 224606_AT, 204619_S_AT, 215646_S_AT, 20162 [symbol] _AT, 210513_S_AT, |
| | 23866 [symbol] _AT, 239452_AT, 37028_AT, 2250 [symbol] 7_AT, 217279_X_AT, 201627_S_AT, 201243_S_AT, |
| | 37152_AT, 201069_AT, 22794 [symbol] _AT, 211924_S_AT, 202708_S_AT, 212948_AT, 201041_S_AT, |
| | 212657_S_AT, 2064 [symbol] _S_AT, 213763_AT, 206171_AT, 220066_AT, 200878_AT, 202014_AT, |
| | 205891_AT, 203665_AT, 204490_S_AT, 201149_S_AT, 239519_AT, 209835_X_AT, 225116_AT, |
| | 206157_AT, 201466_S_AT, 225337_AT, 204035_AT, 203887_S_AT, 225368_AT, 201625_S_AT, |
| | 201012_AT, 212464_S_AT, 216442_X_AT, 204622_X_AT, 21 [symbol] 34_S_AT, 204401_AT, 211661_X_AT, |
| | 222062_AT, 217552_X_AT, 210512_S_AT, 202827_S_AT, [symbol] 7100_AT, 211527_X_AT, 15624 [symbol] 7_AT, |
| | 204465_S_AT, 206573_S_AT, 203140_AT, 202241_AT, 204655_AT, 1552553_A_AT, 2091 [symbol] 9_AT, |
| | 201044_X_AT, 203 [symbol] _AT, 212372_AT, 203935_AT, 1556583_A_AT, 227 [symbol] 7_AT, 1555759_A_AT, |
| | 141929_AT, 211571_AT, 20509 [symbol] _AT |
| GOTERM_{—} | 210136_AT, 205552_S_AT, 216243_S_AT, 204972_AT, 216 [symbol] 34_AT, 203923_S_AT, 201471_S_AT, |
| BP_ALL | 207674_AT, 212657_S_AT, 205067_AT, 202859_X_AT, 220066_AT, 209827_S_AT, 201925_S_AT, |
| | 20274 [symbol] _AT, 211307_S_AT, 203922_S_AT, 229937_X_AT, 223502_S_AT, 206157_AT, 1552914_A_AT, |
| | 20 [symbol] 50 [symbol] _AT, 2029 [symbol] _S_AT, 209392_AT, 224859_AT, 242907_AT, 213293_S_AT, 214211_AT, |
| | 212171_X_AT, 39402_AT, 210233_AT, 1405_I_AT, 209949_AT, 22621 [symbol] _AT, 223501_AT, 2050 [symbol] 9_S_AT, |
| | 200748_S_AT, 211661_X_AT, 222062_AT, 238542_AT, 217552_X_AT, 210512_S_AT, 217078_S_AT, |
| | 221463_AT, 205419_AT, 211527_X_AT, 238581_AT, 210 [symbol] 39_S_AT, 204655_AT, 210513_S_AT, |
| | 203233_AT, 1555759_A_AT, 219434_AT, 205098_AT |
| GOTERM_{—} | 210495_X_AT, 229221_AT, 216243_S_AT, 212014_X_AT, 203923_S_AT, 202708_S_AT, 210916_S_AT, |
| BP_ALL | 212 [symbol] 57_S_AT, 206171_AT, 205067_AT, 202859_X_AT, 220066_AT, 201925_S_AT, 204489_S_AT, |
| | 205891_AT, 203 [symbol] 65_AT, 204490_S_AT, 209835_X_AT, 20 [symbol] 922_S_AT, 206157_AT, 204035_AT, |
| | 205495_S_AT, 20350 [symbol] _AT, 201012_AT, 20 [symbol] 438_S_AT, 212464_S_AT, 216442_X_AT, 39402_AT, |
| | 210233_AT, 1405_I_AT, 21171 [symbol] _X_AT, 200949_AT, 2020 [symbol] _AT, 205099_S_AT, 211 [symbol] 61_X_AT, |
| | 204401_AT, 1557905_S_AT, 22 [symbol] 062_AT, 20 [symbol] 670_AT, 217552_X_AT, 221463_AT, 208436_S_AT, |
| | 209506_AT, 217757_AT, 204 [symbol] 55_AT, 209189_AT, 1552553_A_AT, 203935_AT, 1555759_A_AT, |
| | 2050 [symbol] _AT |
| GOTERM_{—} | 202540_S_AT, 201464_X_AT, 201465_S_AT, 229221_AT, 227069_AT, 219028_AT, 210136_AT, |
| BP_ALL | 212298_AT, 209555_S_AT, 202435_S_AT, 212014_X_AT, 221731_X_AT, 221011_S_AT, 201473_AT, |
| | 203074_AT, 210916_S_AT, 235739_AT, 206472_S_AT, 20 [symbol] 960_S_AT, 203882_AT, 202828_S_AT, |
| | 205067_AT, 242871_AT, 209803_S_AT, 202284_S_AT, 202859_X_AT, 230360_AT, 236561_AT, |
| | 1569149_AT, 217173_S_AT, 204489_S_AT, 216017_S_AT, 204620_S_AT, 212099_AT, |
| | 201565_S_AT, 201147_S_AT, 201590_X_AT, 232649_AT, 204653_AT, 203104_AT, 225115_AT, |
| | 1555832_S_AT, 22 [symbol] 766_AT, 210845_S_AT, 243556_AT, 201005_AT, 208961_S_AT, 233220_AT, |
| | 210095_S_AT, 237252_AT, 160020_AT, 214297_AT, 212171_X_AT, 216248_S_AT, 224967_AT, |
| | 39402_AT, 205463_S_AT, 201566_X_ATT, 22 [symbol] 64_AT, 214866_AT, 202191_S_AT, 229830_AT, |
| | 1552690_A_AT, 1557905_S_AT, 206 [symbol] 35_AT, 21 [symbol] 502_S_AT, 235944_AT, 222670_S_AT, 209047_AT, |
| | 203751_X_AT, 201150_S_AT, 201148_S_AT, 1554992_AT, 209906_AT, 202768_AT, 213281_AT, |
| | 224606_AT, 204619_AT, 215646_S_AT, 210513_S_AT, 1554600_S_AT, 238669_AT, |
| | 204105_S_AT, 204881_S_AT, 225097_AT, 203752_S_AT, 212803_AT, 225557_AT, 217591_AT, |
| | 217279_X_AT, 37152_AT, 201069_AT, 221842_S_AT, 221924_S_AT, 224218_S_AT, 222877_AT, |
| | 241938_AT, 212948_AT, 228083_AT, 202434_S_AT, 206488_S_AT, 213763_AT, 211962_S_AT, |
| | 1566901_AT, 220066_AT, 200878_AT, 204141_AT, 202388_AT, 205891_AT, 204736_S_AT, |
| | 203665_AT, 202436_S_AT, 204490_S_AT, 2049 [symbol] _S_AT, 208816_X_AT, 201149_S_AT, 239519_AT, |
| | 209835_X_AT, 225116_AT, 212190_AT, 201466_S_AT, 212143_S_AT, 204033_AT, 212086_X_AT, |
| | 220484_AT, 203887_S_AT, 212124_AT, 225368_AT, 208937_S_AT, 228762_AT, 222651_S_AT, |
| | 230529_AT, 211986_AT, 201324_AT, 202539_S_AT, 201012_AT, 213503_X_AT, 204622_X_AT, |
| | 227240_AT, 241722_X_AT, 210427_X_AT, 214724_AT, 226218_AT, 228559_S_AT, 204401_AT, |
| | 211661_X_AT, 2026 [symbol] 6_S_AT, 2046 [symbol] 4_AT, 219 [symbol] 45_AT, 21990 [symbol] _AT, 223092_AT, 210512_S_AT, |
| | 202827_S_AT, 205419_AT, 211527_X_AT, 204475_AT, 1562467_AT, 204465_S_AT, 203140_AT, |
| | 220266_S_AT, 220935_S_AT, 1552553_A_AT, 202437_S_AT, 209189_AT, 202068_S_AT, |
| | 203 [symbol] 88_AT, 212372_AT, 203935_AT, 2120 [symbol] 9_AT, 227697_AT, 201995_AT, 15565 [symbol] 3_A_AT, |
| | 229797_AT, 203411_S_AT, 210173_AT, 241929_AT, 202067_S_AT, 211571_S_AT, 215688_AT, |
| | 203320_AT |
| GOTERM_{—} | 229221_AT, 216442_X_AT, 210435_X_AT, 39402_AT, 210233_AT, 216243_S_AT, 1405_I_AT, |
| BP_ALL | 212014_X_AT, 211719_X_AT, 230 [symbol] 23_S_AT, 210916_S_AT, 205099_S_AT, 211661_X_AT, 212657_S_AT, |
| | 1557905_S_AT, 206171_AT, 205067_AT, 217552_X_AT, 202859_X_AT, 20 [symbol] 436_S_AT, 221463_AT, |
| | 201925_S_AT, 20 [symbol] 06_AT, 2044 [symbol] _S_AT, 203665_AT, 204490_S_AT, 217757_AT, 204655_AT, |
| | 1552553_A_AT, 2091 [symbol] 9_AT, 209 [symbol] 35_X_AT, 203922_S_AT, 205157_AT, 204035_AT, 203935_AT, |
| | 203508_AT, 1555759_A_AT, 201012_AT, 2050 [symbol] _AT, 2124 [symbol] 4_S_AT |
| GOTERM_{—} | 201464_X_AT, 202540_S_AT, 22706 [symbol] _AT, 229221_AT, 201465_S_AT, 21902 [symbol] _AT, 210136_AT, |
| BP_ALL | 21225 [symbol] _AT, 212014_X_AT, 221731_X_AT, 201473_AT, 221011_S_AT, 210916_S_AT, 235739_AT, |
| | 206472_S_AT, 20 [symbol] 960_S_AT, 202 [symbol] 28_S_AT, 209 [symbol] 0 [symbol] _S_AT, 242 [symbol] 71_AT, 205067_AT, 2022 [symbol] 4_S_AT, |
| | 236561_AT, 230360_AT, 202859_X_AT, 1569149_AT, 20 [symbol] _S_AT, 216017_S_AT, 204620_S_AT, |
| | 212099_AT, 201147_S_AT, 201565_S_AT, 201590_X_AT, 232 [symbol] 49_AT, 204653_AT, 203104_AT, |
| | 225115_AT, 1555 [symbol] 32_S_AT, 210845_S_AT, 243556_AT, 201005_AT, 208961_S_AT, 233220_AT, |
| | 210095_S_AT, 237252_AT, 160020_AT, 224967_AT, 216248_S_AT, 234297_AT, 212171_X_AT, |
| | 39402_AT, 2054 [symbol] 3_S_AT, 201566_X_AT, 214 [symbol] 66_AT, 22 [symbol] 964_AT, 202191_S_AT, 229 [symbol] 30_AT, |
| | 1557905_S_AT, 21 [symbol] 502_S_AT, 206 [symbol] 35_AT, 222670_S_AT, 201150_S_AT, 20 [symbol] 751_X_AT, 20114 [symbol] _S_AT, |
| | 1554992_AT, 202768_AT, 2132 [symbol] 1_AT, 224606_AT, 204619_S_AT, 2156 [symbol] 6_S_AT, 210513_S_AT, |
| | 1554600_S_AT, 204105_S_AT, 204 [symbol] 1_S_AT, 225097_AT, 20 [symbol] 752_S_AT, 212 [symbol] 03_AT, 225557_AT, |
| | 217279_X_AT, 217591_AT, 37152_AT, 201069_AT, 221841_S_AT, 211924_S_AT, 224218_S_AT, |
| | 222877_AT, 24193 [symbol] _AT, 213763_AT, 211962_S_AT, 1566901_AT, 200 [symbol] _AT, 204141_AT, |
| | 204736_S_AT, 203665_AT, 204490_S_AT, 204998_S_AT, 208816_X_AT, 201149_S_AT, 239519_AT, |
| | 209 [symbol] 35_X_AT, 225116_AT, 232150_AT, 201466_S_AT, 212143_S_AT, 204035_AT, 2120 [symbol] 6_X_AT, |
| | 2204 [symbol] 4_AT, 203 [symbol] 7_S_AT, 212134_AT, 225368_AT, 208937_S_AT, 228762_AT, 222651_S_AT, |
| | 2219 [symbol] _AT, 230529_AT, 201324_AT, 201012_AT, 202539_S_AT, 213503_X_AT, 204622_X_AT, |
| | 227240_AT, 241722_X_AT, 210427_X_AT, 214724_AT, 22621 [symbol] _AT, 218559_S_AT, 2026 [symbol] 6_S_AT, |
| | 204684_AT, 219 [symbol] 45_AT, 219906_AT, 202 [symbol] 27_S_AT, 210512_S_AT, 223092_AT, 205419_AT, |
| | 211527_X_AT, 1562467_AT, 204465_S_AT, 203140_AT, 220266_S_AT, 220935_S_AT, 209189_AT, |
| | 212372_AT, 203 [symbol] _AT, 203935_AT, 2120 [symbol] 9_AT, 201996_AT, 227697_AT, 229797_AT, 203411_S_AT, |
| | 210173_AT, 211571_S_AT, 2136 [symbol] _AT, 203320_AT |
| GOTERM_{—} | 202540_S_AT, 210495_X_AT, 229221_AT, 37152_AT, 212298_AT, 212014_X_AT, 211924_S_AT, |
| BP_ALL | 221732_X_AT, 222877_AT, 210915_S_AT, 204268_AT, 235735_AT, 223220_S_AT, 202 [symbol] 2 [symbol] _S_AT, |
| | 205067_AT, 202859_X_AT, 236561_AT, 209 [symbol] 27_S_AT, 204489_S_AT, 204490_S_AT, 204620_S_AT, |
| | 239519_AT, 209835_X_AT, 204035_AT, 208937_S_AT, 209392_AT, 210 [symbol] 45_S_AT, 202539_S_AT, |
| | 212464_S_AT, 160020_AT, 216442_X_AT, 216248_S_AT, 39402_AT, 204622_X_AT, 2054 [symbol] 3_S_AT, |
| | 1405_I_AT, 214 [symbol] 66_AT, 211719_X_AT, 229 [symbol] 30_AT, 205099_S_AT, 211661_X_AT, 1557935_S_AT, |
| | 200921_S_AT, 202827_S_AT, 221463_AT, 200920_S_AT, 210839_S_AT, 209906_AT, 204655_AT, |
| | 21564 [symbol] _S_AT, 204619_S_AT, 212372_AT, 20 [symbol] 35_AT, 204105_S_AT, 1555759_A_AT, 217279_X_AT, |
| | 211571_S_AT, 205098_AT |
| GOTERM_{—} | 202540_S_AT, 201464_X_AT, 210495_X_AT, 201465_S_AT, 229221_AT, 227069_AT, 219028_AT, |
| BP_ALL | 210136_AT, 212298_AT, 212014_X_AT, 221731_X_AT, 221011_S_AT, 201473_AT, 210916_S_AT, |
| | 201471_S_AT, 235739_AT, 206472_S_AT, 208960_S_AT, 202828_S_AT, 205067_AT, 242871_AT, |
| | 209 [symbol] 03_S_AT, 226648_AT, 2022 [symbol] 4_S_AT, 202 [symbol] 59_X_AT, 230360_AT, 236561_AT, 156 [symbol] 149_AT, |
| | 204489_S_AT, 216017_S_AT, 208891_AT, 204620_S_AT, 212099_AT, 201565_S_AT, 201147_S_AT, |
| | 201590_X_AT, 232649_AT, 204653_AT, 203104_AT, 225115_AT, 1555 [symbol] 32_S_AT, 210845_S_AT, |
| | 243556_AT, 201005_AT, 20 [symbol] 961_S_AT, 233220_AT, 210095_S_AT, 237252_AT, 160020_AT, |
| | 214297_AT, 212171_X_AT, 216248_S_AT, 224967_AT, 39402_AT, 205463_S_AT, 1405_I_AT, |
| | 201566_X_AT, 22 [symbol] 964_AT, 211719_X_AT, 214 [symbol] 66_AT, 202191_AT, 229 [symbol] 30_AT, 1557905_S_AT, |
| | 21 [symbol] 502_S_AT, 206835_AT, 222670_S_AT, 203751_X_AT, 201150_S_AT, 201148_S_AT, 155 [symbol] 992_AT, |
| | 202768_AT, 2132 [symbol] 1_AT, 224606_AT, 204619_S_AT, 215646_S_AT, 210613_S_AT, 1554600_S_AT, |
| | 23 [symbol] 669_AT, 204105_S_AT, 204 [symbol] 1_S_AT, 225097_AT, 20 [symbol] 752_S_AT, 212803_AT, 225557_AT, |
| | 217591_AT, 217279_X_AT, 37152_AT, 201069_AT, 221841_S_AT, 211924_S_AT, 224218_S_AT, |
| | 222 [symbol] 77_AT, 24193 [symbol] _AT, 213763_AT, 211962_S_AT, 1566001_AT, 200 [symbol] 78_AT, 204141_AT, 202388_AT, |
| | 20 [symbol] 736_S_AT, 203665_AT, 204490_S_AT, 20499 [symbol] _S_AT, 20 [symbol] 16_X_AT, 201149_S_AT, 23 [symbol] 19_AT, |
| | 209835_X_AT, 225116_AT, 212190_AT, 201466_S_AT, 212143_S_AT, 204035_AT, 2120 [symbol] _X_AT, |
| | 220484_AT, 203887_S_AT, 212124_AT, 225368_AT, 208537_S_AT, 228762_AT, 222651_S_AT, |
| | 230529_AT, 211986_AT, 201324_AT, 202539_S_AT, 301012_AT, 212464_S_AT, 216442_X_AT, |
| | 213503_X_AT, 204622_X_AT, 227240_AT, 241722_X_AT, 210427_X_AT, 214724_AT, 226218_AT, |
| | 218559_S_AT, 202686_S_AT, 204684_AT, 20 [symbol] _S_AT, 219 [symbol] 45_AT, 21990 [symbol] _AT, 223092_AT, |
| | 210512_S_AT, 202827_S_AT, 205419_AT, 211527_X_AT, 1562467_AT, 204465_S_AT, 203140_AT, |
| | 20 [symbol] 92_S_AT, 220266_S_AT, 220935_S_AT, 204655_AT, 2091 [symbol] 9_AT, 203 [symbol] _AT, 212372_AT, |
| | 203935_AT, 212089_AT, 227637_AT, 201995_AT, 229797_AT, 1555759_A_AT, 203411_S_AT, |
| | 210173_AT, 211571_S_AT, 2156 [symbol] 8_AT, 203320_AT |
| GOTERM_{—} | 160020_AT, 3 [symbol] 402_AT, 2054 [symbol] 3_S_AT, 216243_S_AT, 1405_I_AT, 201473_AT, 229 [symbol] 30_AT, |
| BP_ALL | 201041_S_AT, 212657_S_AT, 202 [symbol] 28_S_AT, 205067_AT, 2022 [symbol] 4_S_AT, 202827_S_AT, 236561_AT, |
| | 209047_AT, 201150_S_AT, 217173_S_AT, 201148_S_AT, 203665_AT, 217757_AT, 204655_AT, |
| | 201149_S_AT, 2091 [symbol] _AT, 201147_S_AT, 202068_S_AT, 201044_X_AT, 23 [symbol] 669_AT, 201313_AT, |
| | 227697_AT, 1555759_A_AT, 217279_X_AT, 202067_S_AT, 207819_S_AT |
| GOTERM_{—} | 237252_AT, 216442_X_AT, 210495_X_AT, 201069_AT, 212171_X_AT, 39402_AT, 205463_S_AT, |
| CC_ALL | 216243_S_AT, 1405_I_AT, 211719_X_AT, 223501_AT, 229830_AT, 212657_S_AT, 203940_S_AT, |
| | 227265_AT, 23 [symbol] 542_AT, 203548_S_AT, 205067_AT, 219908_AT, 210512_S_AT, 202 [symbol] 59_X_AT, |
| | 221463_AT, 226545_AT, 211527_X_AT, 209827_S_AT, 217273_S_AT, 203665_AT, 217757_AT, |
| | 204655_AT, 210513_S_AT, 202068_S_AT, 21219_AT, 223502_S_AT, 203 [symbol] 8_AT, 212143_S_AT, |
| | 204035_AT, 203887_S_AT, 202756_S_AT, 204 [symbol] 34_AT, 205495_S_AT, 1555759_A_AT, 216546_S_AT, |
| | 202067_S_AT, 210095_S_AT, 212464_S_AT |
| GOTERM_{—} | 201464_X_AT, 202540_S_AT, 210495_X_AT, 227069_AT, 229221_AT, 201465_S_AT, 210136_AT, |
| BP_ALL | 212298_AT, 212014_X_AT, 221731_X_AT, 201473_AT, 210916_S_AT, 235739_AT, 206472_S_AT, |
| | 20 [symbol] 960_S_AT, 202 [symbol] 2 [symbol] _S_AT, 205067_AT, 209 [symbol] 03_S_AT, 236561_AT, 230360_AT, 202859_X_AT, |
| | 202284_S_AT, 156 [symbol] 149_AT, 204489_S_AT, 21 [symbol] 017_S_AT, 204620_S_AT, 212099_AT, 201147_S_AT, |
| | 201565_S_AT, 201590_X_AT, 232649_AT, 204653_AT, 1555832_S_AT, 210845_S_AT, 243 [symbol] 6_AT, |
| | 201005_AT, 208961_S_AT, 233220_AT, 2100 [symbol] 5_S_AT, 250020_AT, 224967_AT, 21624 [symbol] _S_AT, |
| | 214297_AT, 212171_X_AT, 39402_AT, 205463_S_AT, 201566_X_AT, 214866_AT, 228964_AT, |
| | 211719_X_AT, 202191_S_AT, 229830_AT, 1557905_S_AT, 21 [symbol] 502_S_AT, 206835_AT, 222670_S_AT, |
| | 201150_S_AT, 203751_X_AT, 20114 [symbol] _S_AT, 1554992_AT, 2132 [symbol] 1_AT, 224 [symbol] 06_AT, 20461 [symbol] _S_AT, |
| | 215646_S_AT, 210513_S_AT, 1554600_S_AT, 204105_S_AT, 204 [symbol] 1_S_AT, 203752_S_AT, 212 [symbol] 03_AT, |
| | 225557_AT, 217279_X_AT, 37152_AT, 201069_AT, 221 [symbol] 41_S_AT, 211924_S_AT, 224218_S_AT, |
| | 222877_AT, 241938_AT, 211962_AT, 1566901_AT, 200 [symbol] 7 [symbol] _AT, 204141_AT, 204736_S_AT, |
| | 203665_AT, 204490_S_AT, 204998_S_AT, 20 [symbol] 16_X_AT, 20114 [symbol] _S_AT, 239519_AT, [symbol] 09 [symbol] 35_X_AT, |
| | 212190_AT, 201466_S_AT, 212143_S_AT, 204035_AT, 2120 [symbol] 6_X_AT, 220484_AT, 212124_AT, |
| | 20 [symbol] 37_S_AT, 22 [symbol] 762_AT, 222651_S_AT, 211986_AT, 230529_AT, 201324_AT, 201012_AT, |
| | 202539_S_AT, 212464_S_AT, 216442_X_AT, 213503_X_AT, 204622_X_AT, 227240_AT, 210427_X_AT |
| | 226218_AT, 218559_S_AT, 2026 [symbol] 6_S_AT, 2046 [symbol] 4_AT, 2198 [symbol] 5_AT, 210512_S_AT, 223092_AT, |
| | 202 [symbol] 27_S_AT, 205419_AT, 211527_X_AT, 1562467_AT, 204465_S_AT, 203140_AT, 220266_S_AT, |
| | 220935_S_AT, 209189_AT, 212372_AT, 203935_AT, 2120 [symbol] _AT, 201995_AT, 2276 [symbol] 7_AT, 229797_AT, |
| | 203411_S_AT, 210173_AT, 212571_S_AT, 2156 [symbol] _AT, 203 [symbol] 0_AT |
| GOTERM_{—} | 21902 [symbol] _AT, 212171_X_AT, 213503_X_AT, 39402_AT, 205463_S_AT, 227948_AT, 1405_I_AT, |
| BP_ALL | 210427_X_AT, 226218_AT, 223501_AT, 229830_AT, 204923_AT, 213763_AT, 222062_AT, 205067_AT, |
| | 210512_S_AT, 202 [symbol] 59_X_AT, 217552_X_AT, 208436_S_AT, 220066_AT, 211527_X_AT, 209827_S_AT, |
| | 203939_AT, 201925_S_AT, 209906_AT, 203665_AT, 205 [symbol] 91_AT, 20 [symbol] 140_AT, 217757_AT, |
| | 20 [symbol] 16_X_AT, 204655_AT, 210513_S_AT, 201590_X_AT, 225116_AT, 223502_S_AT, 1552914_A_AT, |
| | 204035_AT, 225115_AT, 203233_AT, 2276 [symbol] 7_AT, 225097_AT, 203508_AT, 225368_AT, 155575 [symbol] _A_AT, |
| | 224859_AT, 233220_AT |
| GOTERM_{—} | 210136_AT, 205552_S_AT, 204972_AT, 216243_S_AT, 216834_AT, 203923_S_AT, 201471_S_AT, |
| BP_ALL | 207674_AT, 212657_S_AT, 20 [symbol] 960_S_AT, 205067_AT, 202859_X_AT, 236561_AT, 220066_AT, |
| | 209827_S_AT, 200 [symbol] 78_AT, 201925_S_AT, 202748_AT, 211307_S_AT, 201565_S_AT, 203922_S_AT, |
| | 229937_X_AT, 223502_S_AT, 206157_AT, 1552914_A_AT, 204035_AT, 1555832_S_AT, 203508_AT, |
| | 2029 [symbol] _S_AT, 209392_AT, 224859_AT, 208961_S_AT, 242907_AT, 213293_S_AT, 214211_AT, |
| | 212171_X_AT, 39402_AT, 210233_AT, 1405_I_AT, 201566_X_AT, 209949_AT, 226218_AT, 223501_AT, |
| | 200748_S_AT, 205099_S_AT, 211661_X_AT, 22262_AT, 219 [symbol] 45_AT, 23 [symbol] 542_AT, 210512_S_AT, |
| | 217552_X_AT, 221453_AT, 217078_S_AT, 205419_AT, 211527_X_AT, 23 [symbol] 581_AT, 210839_S_AT, |
| | 209906_AT, 203140_AT, 224606_AT, 204655_AT, 210513_S_AT, 203233_AT, 1555759_A_AT, |
| | 219434_AT, 203320_AT, 2050 [symbol] _AT |
| GOTERM_{—} | 160020_AT, 210495_X_AT, 216442_X_AT, 229221_AT, 201069_AT, 212171_X_AT, 213503_X_AT, |
| CC_ALL | 212014_X_AT, 210427_X_AT, 21719_X_AT, 221731_X_AT, 210916_S_AT, 1557905_S_AT, |
| | 202828_S_AT, 215944_AT, 203548_S_AT, 210512_S_AT, 202827_S_AT, 211527_X_AT, 204475_AT, |
| | 201150_S_AT, 20114 [symbol] _S_AT, 204489_S_AT, 213428_S_AT, 204358_AT, 204490_S_AT, 208816_X_AT, |
| | 204620_S_AT, 229004_AT, 201149_S_AT, 210513_S_AT, 201147_S_AT, 215646_S_AT, 204619_S_AT, |
| | 209835_X_AT, 201590_X_AT, 202756_S_AT, 22 [symbol] 73_AT, 226546_S_AT, 217279_X_AT, 211571_S_AT, |
| | 212 [symbol] 4_S_AT |
| GOTERM_{—} | 160020_AT, 201464_X_AT, 201465_S_AT, 214297_AT, 212298_AT, 213503_X_AT, 212171_X_AT, |
| BP_ALL | 39402_AT, 205463_S_AT, 210427_X_AT, 222877_AT, 229830_AT, 202828_S_AT, 205067_AT, |
| | 202827_S_AT, 202859_X_AT, 210512_S_AT, 211527_X_AT, 200 [symbol] 78_AT, 203665_AT, 204736_S_AT, |
| | 2132 [symbol] 1_AT, 212099_AT, 208816_X_AT, 239519_AT, 210513_S_AT, 201590_X_AT, 2014 [symbol] 6_S_AT, |
| | 20 [symbol] 35_AT, 204035_AT, 20 [symbol] 37_S_AT, 217279_X_AT |
| GOTERM_{—} | 201464_X_AT, 237252_AT, 201465_S_AT, 39402_AT, 1405_I_AT, 20270 [symbol] _S_AT, 202086_AT, |
| BP_ALL | 211661_X_AT, 222062_AT, 203882_AT, 239818_X_AT, 205067_AT, 220066_AT, 208436_S_AT, |
| | 214453_S_AT, 202241_AT, 213281_AT, 204655_AT, 209189_AT, 1552553_A_AT, 211676_S_AT, |
| | 229937_X_AT, 201466_S_AT, 206157_AT, 203 [symbol] _AT, 227697_AT, 203 [symbol] 7_S_AT, 205495_S_AT, |
| | 1555759_A_AT, 20843 [symbol] _S_AT, 213293_S_AT |
| GOTERM_{—} | 202540_S_AT, 160020_AT, 229221_AT, 210495_X_AT, 216442_X_AT, 37152_AT, 212298_AT, |
| BP_ALL | 21624 [symbol] _S_AT, 39402_AT, 204622_X_AT, 1405_I_AT, 212014_X_AT, 221731_X_AT, 211719_X_AT, |
| | 222 [symbol] 77_AT, 210916_S_AT, 204268_AT, 235739_AT, 223220_S_AT, 1557905_S_AT, 200921_S_AT, |
| | 202 [symbol] 28_S_AT, 205067_AT, 202 [symbol] 59_X_AT, 202827_S_AT, 236561_AT, 200920_S_AT, 209827_S_AT |
| | 2044 [symbol] _S_AT, 204490_S_AT, 204620_S_AT, 204655_AT, 239519_AT, 204619_S_AT, 215646_S_AT, |
| | 209835_X_AT, 212372_AT, 204035_AT, 203 [symbol] 5_AT, 204205_S_AT, 208937_S_AT, 1555759_A_AT, |
| | 217279_X_AT, 211571_S_AT, 202539_S_AT, 212464_S_AT |
| GOTERM_{—} | 202540_S_AT, 223723_AT, 227069_AT, 219028_AT, 212298_AT, 204872_AT, 212014_X_AT, 221731_X_AT, 216 [symbol] 34_AT, |
| CC_ALL | 203923_S_AT, 219412_AT, 207674_AT, 217997_AT, 209 [symbol] 03_S_AT, 23 [symbol] 681_AT, 228648_AT, 226545_AT, 203729_AT, |
| | 217173_S_AT, 2044 [symbol] 9_S_AT, 205505_AT, 204620_S_AT, 220307_AT, 223303_AT, 211307_S_AT, 2160 [symbol] 0_S_AT, |
| | 201590_X_AT, 232649_AT, 203922_S_AT, 2186 [symbol] 6_S_AT, 221825_AT, 1552914_A_AT, 203104_AT, 223562_S_AT, |
| | 202756_S_AT, 228766_AT, 224674_AT, 214211_AT, 214581_X_AT, 20 [symbol] 926_AT, 224967_AT, 63825_AT, 205463_S_AT, |
| | 211719_X_AT, 209949_AT, 223501_AT, 229 [symbol] 30_AT, 205099_S_AT, 1557905_S_AT, 209670_AT, 215 [symbol] 36_S_AT, 235944_AT, |
| | 23 [symbol] 542_AT, 217074_S_AT, 209047_AT, 240076_AT, 217996_AT, 1554992_AT, 221840_AT, 201626_AT, 219926_AT, |
| | 211676_S_AT, 225 [symbol] 42_AT, 1554600_S_AT, 23 [symbol] 669_AT, 204257_AT, 203233_AT, 2249 [symbol] 9_AT, 204105_S_AT, 239761_AT, |
| | 201243_S_AT, 204912_AT, 23863 [symbol] _AT, 222 [symbol] 77_AT, 24193 [symbol] _AT, 209079_X_AT, 212062_AT, 22 [symbol] 0 [symbol] 3_AT, 202434_S_AT, |
| | 204222_S_AT, 2064 [symbol] _S_AT, 220066_AT, 228640_AT, 205542_AT, 204359_AT, 203665_AT, 204736_S_AT, 205891_AT, |
| | 20274 [symbol] _AT, 208816_X_AT, 239519_AT, 209835_X_AT, 208161_S_AT, 218856_AT, 225116_AT, 204715_AT, 223502_S_AT, |
| | 256345_AT, 2120 [symbol] 6_AT, 2239 [symbol] 9_AT, 203 [symbol] 7_S_AT, 2029 [symbol] _S_AT, 204774_AT, 202539_S_AT, 227240_AT, |
| | 210427_X_AT, 2621 [symbol] _AT, 204661_AT, 20074 [symbol] _S_AT, 225 [symbol] 09_AT, 204401_AT, 2026 [symbol] 6_S_AT, 222062_AT, 210512_S_AT, |
| | 2251 [symbol] _AT, 217552_X_AT, 211527_X_AT, 202071_AT, 224990_AT, 225325_AT, 220313_AT, 20571 [symbol] _AT, 203935_AT, |
| | 210258_AT, 15565 [symbol] 3_A_AT, 201995_AT, 229797_AT, 226302_AT, 203411_S_AT, 21017 [symbol] _AT, 2156 [symbol] _AT, 20509 [symbol] _AT, |
| | 2104 [symbol] 5_X_AT, 229221_AT, 210136_AT, 209555_S_AT, 202435_S_AT, 211066_X_AT, 216971_S_AT, 210916_S_AT, |
| | 2244 [symbol] 0_S_AT, 219225_AT, 202828_S_AT, 214255_AT, 242871_AT, 236561_AT, 230360_AT, 201925_S_AT, 228490_AT, |
| | 207610_S_AT, 212099_AT, 213577_AT, 229937_X_AT, 214830_AT, 227052_AT, 225115_AT, 212977_AT, 203508_AT, |
| | 230925_AT, 210 [symbol] 45_S_AT, 243556_AT, 201005_AT, 206033_S_AT, 201242_S_AT, 233220_AT, 207819_S_AT, 242907_AT, |
| | 209122_AT, 160020_AT, 237252_AT, 212171_X_AT, 214297_AT, 210133_AT, 22 [symbol] 910_AT, 214 [symbol] 66_AT, 205534_AT, |
| | 34210_AT, 205566_AT, 209994_S_AT, 1552690_A_AT, 228754_AT, 21767 [symbol] _AT, 205717_X_AT, 23 [symbol] 5 [symbol] 1_AT, 201739_AT, |
| | 209906_AT, 20 [symbol] 218_AT, 210513_S_AT, 204619_S_AT, 215646_S_AT, 25920_AT, 211030_S_AT, 20 [symbol] 121_S_AT, |
| | 235295_AT, 201313_AT, 239451_AT, 204881_S_AT, 225097_AT, 219994_AT, 217279_X_AT, 201125_S_AT, 201627_S_AT, |
| | 201069_AT, 211924_S_AT, 203217_S_AT, 213763_AT, 206171_AT, 241954_AT, 203939_AT, 243 [symbol] 94_AT, 21342 [symbol] _S_AT, |
| | 202436_S_AT, 204490_S_AT, 219386_S_AT, 209921_AT, 225337_AT, 2204 [symbol] 4_AT, 209146_AT, 22536 [symbol] _AT, 241773_AT, |
| | 22 [symbol] 762_AT, 209392_AT, 224859_AT, 201625_S_AT, 201324_AT, 209993_AT, 201012_AT, 212464_S_AT, 216442_X_AT, |
| | 2271 [symbol] 0_AT, 213503_X_AT, 241722_X_AT, 228708_AT, 214724_AT, 227107_AT, 211661_X_AT, 225207_AT, 226931_AT, |
| | 2193 [symbol] 5_AT, 203548_S_AT, 202 [symbol] 27_S_AT, 223092_AT, 205419_AT, [symbol] 7100_AT, 1562467_AT, 210 [symbol] 39_S_AT, 235299_AT, |
| | 224701_AT, 219332_AT, 202437_S_AT, 20206 [symbol] _S_AT, 212372_AT, 203 [symbol] _AT, 22379 [symbol] _AT, 212096_S_AT, 2120 [symbol] 9_AT, |
| | 22 [symbol] 696_AT, 218223_S_AT, 241929_AT, 202067_S_AT, 219434_AT, 211571_S_AT |
| KEGG_{—} | 214581_X_AT, 204912_AT, 212171_X_AT, 39402_AT, 205463_S_AT, 210233_AT, 1405_I_AT, |
| PATHWAY | 226218_AT, 223501_AT, 229830_AT, 205099_S_AT, 205067_AT, 202859_X_AT, 236561_AT, |
| | 210512_S_AT, 221463_AT, 211527_X_AT, 204655_AT, 210513_S_AT, 211676_S_AT, 218856_AT, |
| | 223502_S_AT, 203935_AT, 203104_AT, 203233_AT, 203508_AT, 204436_AT, 1555759_A_AT, |
| | 205098_AT |
| GOTERM_{—} | 201464_X_AT, 210495_X_AT, 229221_AT, 201465_S_AT, 201069_AT, 212298_AT, 221841_S_AT, |
| BP_ALL | 211924_S_AT, 212014_X_AT, 221731_X_AT, 201473_AT, 222877_AT, 241938_AT, 210916_S_AT, |
| | 235739_AT, 206472_S_AT, 211962_S_AT, 202828_S_AT, 1556901_AT, 209803_S_AT, 205067_AT, |
| | 202284_S_AT, 202859_X_AT, 236561_AT, 200878_AT, 204489_S_AT, 216017_AT, 203665_AT, |
| | 204736_S_AT, 204490_S_AT, 204620_S_AT, 208816_X_AT, 212099_AT, 201149_S_AT, 201147_S_AT, |
| | 239519_AT, 201590_X_AT, 209835_X_AT, 204653_AT, 201466_S_AT, 204035_AT, 212124_AT, |
| | 208937_S_AT, 210845_S_AT, 228762_AT, 201005_AT, 212464_S_AT, 160020_AT, 216442_X_AT, |
| | 216248_S_AT, 214297_AT, 213503_X_AT, 212171_X_AT, 39402_AT, 204622_X_AT, 205463_S_AT, |
| | 210427_X_AT, 214866_AT, 211719_X_AT, 226218_AT, 229830_AT, 202191_S_AT, 218559_S_AT, |
| | 1557905_S_AT, 202686_S_AT, 202827_S_AT, 222670_S_AT, 210512_S_AT, 211527_X_AT, |
| | 201150_S_AT, 204465_S_AT, 201148_S_AT, 203140_AT, 220266_S_AT, 213281_AT, 215646_S_AT, |
| | 204619_S_AT, 210513_S_AT, 212372_AT, 203935_AT, 204105_S_AT, 227697_AT, 201995_AT, |
| | 212803_AT, 225557_AT, 210173_AT, 217279_X_AT, 211571_S_AT |
| GOTERM_{—} | 201464_X_AT, 201465_S_AT, 229221_AT, 202435_S_AT, 216243_S_AT, 212014_X_AT, 201473_AT, |
| BP_ALL | 210916_S_AT, 235739_AT, 202828_S_AT, 205067_AT, 202859_X_AT, 236561_AT, 202284_S_AT, |
| | 209827_S_AT, 217173_S_AT, 204489_S_AT, 201565_S_AT, 201147_S_AT, 213577_AT, 213562_S_AT, |
| | 210845_S_AT, 201242_S_AT, 233220_AT, 207819_S_AT, 209122_AT, 237252_AT, 160020_AT, |
| | 212171_X_AT, 216248_S_AT, 39402_AT, 205463_S_AT, 1405_I_AT, 201566_X_AT, 214866_AT, |
| | 204058_AT, 229830_AT, 205099_S_AT, 209994_S_AT, 1557905_S_AT, 239818_X_AT, 217678_AT, |
| | 209047_AT, 221463_AT, 201150_S_AT, 201148_S_AT, 209906_AT, 209218_AT, 204059_S_AT, |
| | 217757_AT, 213281_AT, 210513_S_AT, 238669_AT, 235295_AT, 201313_AT, 239451_AT, 37028_AT, |
| | 217279_X_AT, 201243_S_AT, 201069_AT, 221841_S_AT, 211924_S_AT, 222877_AT, 201041_S_AT, |
| | 202434_S_AT, 212657_S_AT, 220066_AT, 202014_AT, 200878_AT, 203665_AT, 204490_S_AT, |
| | 202436_S_AT, 201149_S_AT, 209835_X_AT, 209922_AT, 204715_AT, 201466_S_AT, 204035_AT, |
| | 203887_S_AT, 208937_S_AT, 209392_AT, 209993_AT, 204622_X_AT, 241722_X_AT, 218934_S_AT, |
| | 227107_AT, 211661_X_AT, 210512_S_AT, 202827_S_AT, 211527_X_AT, 1562467_AT, 210839_S_AT, |
| | 220266_S_AT, 202241_AT, 204655_AT, 209189_AT, 202437_S_AT, 202068_S_AT, 201044_X_AT, |
| | 203888_AT, 1556583_A_AT, 227697_AT, 1555759_A_AT, 202067_S_AT, 205098_AT |
| GOTERM_{—} | 201464_X_AT, 201465_S_AT, 229221_AT, 202435_S_AT, 216243_S_AT, 212014_X_AT, 201473_AT, |
| BP_ALL | 210916_S_AT, 201041_S_AT, 235739_AT, 202434_S_AT, 212657_S_AT, 202828_S_AT, 205067_AT, |
| | 202284_S_AT, 236561_AT, 220066_AT, 202014_AT, 217173_S_AT, 204489_S_AT, 203665_AT, |
| | 204490_S_AT, 202436_S_AT, 201149_S_AT, 201147_S_AT, 201565_S_AT, 209835_X_AT, 213577_AT, |
| | 204715_AT, 201466_S_AT, 213562_S_AT, 203887_S_AT, 208937_S_AT, 233220_AT, 207819_S_AT, |
| | 209122_AT, 160020_AT, 237252_AT, 216248_S_AT, 39402_AT, 205463_S_AT, 204622_X_AT, |
| | 241722_X_AT, 1405_I_AT, 201566_X_AT, 218934_S_AT, 204058_AT, 229830_AT, 227107_AT, |
| | 211661_X_AT, 1557905_S_AT, 239818_X_AT, 202827_S_AT, 209047_AT, 1563467_AT, 201150_S_AT, |
| | 201148_S_AT, 209218_AT, 204059_S_AT, 217757_AT, 213281_AT, 202241_AT, 204655_AT, |
| | 209189_AT, 202437_S_AT, 202068_S_AT, 201044_X_AT, 238669_AT, 203888_AT, 235295_AT, |
| | 201313_AT, 1556583_A_AT, 227697_AT, 37028_AT, 1555759_A_AT, 217279_X_AT, 202067_S_AT |
| GOTERM_{—} | 201464_X_AT, 202540_S_AT, 229221_AT, 227069_AT, 201465_S_AT, 212298_AT, 212014_X_AT, |
| BP_ALL | 201473_AT, 210916_S_AT, 208960_S_AT, 206472_S_AT, 202828_S_AT, 205067_AT, 209803_S_AT, |
| | 236561_AT, 202859_X_AT, 202284_S_AT, 204489_S_AT, 216017_S_AT, 212099_AT, 201565_S_AT, |
| | 201147_S_AT, 201590_X_AT, 204653_AT, 1555832_S_AT, 210845_S_AT, 208961_S_AT, 160020_AT, |
| | 212171_X_AT, 214297_AT, 224967_AT, 39402_AT, 205463_S_AT, 201566_X_AT, 228964_AT, |
| | 214866_AT, 229830_AT, 1557905_S_AT, 206835_AT, 222670_S_AT, 201150_S_AT, 201148_S_AT, |
| | 213281_AT, 224606_AT, 210513_S_AT, 1554600_S_AT, 204881_S_AT, 212803_AT, 225557_AT, |
| | 217279_X_AT, 201069_AT, 37152_AT, 221841_S_AT, 211924_S_AT, 222877_AT, 241938_AT, |
| | 211962_S_AT, 1566901_AT, 200878_AT, 204736_S_AT, 203665_AT, 204490_S_AT, 208816_X_AT, |
| | 201149_S_AT, 239519_AT, 209835_X_AT, 201466_S_AT, 204035_AT, 212086_X_AT, 220484_AT, |
| | 212124_AT, 208937_S_AT, 228762_AT, 230529_AT, 201324_AT, 202539_S_AT, 201012_AT, |
| | 213503_X_AT, 210427_X_AT, 226218_AT, 218559_S_AT, 202686_S_AT, 219845_AT, 210512_S_AT, |
| | 202827_S_AT, 205419_AT, 211527_X_AT, 1562467_AT, 204465_S_AT, 203140_AT, 220255_S_AT, |
| | 220935_S_AT, 212372_AT, 203935_AT, 212089_AT, 201995_AT, 227697_AT, 229797_AT, |
| | 203411_S_AT, 210173_AT, 203320_AT |
| GOTERM_{—} | 201464_X_AT, 213891_S_AT, 227069_AT, 201465_S_AT, 219028_AT, 212298_AT, 210136_AT, |
| BP_ALL | 216834_AT, 201471_S_AT, 235739_AT, 202828_S_AT, 205067_AT, 236561_AT, 202859_X_AT, |
| | 202284_S_AT, 201170_S_AT, 203729_AT, 228490_AT, 216017_S_AT, 212099_AT, 201147_S_AT, |
| | 201565_S_AT, 201590_X_AT, 203753_AT, 221815_AT, 1552914_A_AT, 225115_AT, 222146_S_AT, |
| | 203508_AT, 205312_AT, 201005_AT, 233220_AT, 210095_S_AT, 214211_AT, 160020_AT, |
| | 212171_X_AT, 216248_S_AT, 63825_AT, 39402_AT, 205463_S_AT, 1405_I_AT, 201566_X_AT, |
| | 228964_AT, 223501_AT, 223398_AT, 229830_AT, 212387_AT, 205566_AT, 218502_S_AT, 206835_AT, |
| | 239818_X_AT, 222670_S_AT, 208436_S_AT, 200920_S_AT, 201150_S_AT, 201148_S_AT, 202768_AT, |
| | 217757_AT, 213281_AT, 210513_S_AT, 238669_AT, 203233_AT, 239451_AT, 212386_AT, 225097_AT, |
| | 219257_S_AT, 212803_AT, 217591_AT, 217279_X_AT, 37152_AT, 221841_S_AT, 224218_S_AT, |
| | 213763_AT, 1566901_AT, 220066_AT, 203939_AT, 202388_AT, 205891_AT, 203665_AT, 204998_S_AT, |
| | 208816_X_AT, 201149_S_AT, 239519_AT, 225116_AT, 201466_S_AT, 223502_S_AT, 225337_AT, |
| | 212143_S_AT, 204035_AT, 225368_AT, 202988_S_AT, 208937_S_AT, 222651_S_AT, 224859_AT, |
| | 201012_AT, 205681_AT, 213503_X_AT, 204622_X_AT, 241722_X_AT, 210427_X_AT, 226218_AT, |
| | 200748_S_AT, 218559_S_AT, 222062_AT, 203940_S_AT, 200921_S_AT, 219845_AT, 219908_AT, |
| | 210512_S_AT, 202827_S_AT, 87100_AT, 211527_X_AT, 203140_AT, 220266_S_AT, 202241_AT, |
| | 204655_AT, 203935_AT, 210258_AT, 227697_AT, 1555759_A_AT, 226302_AT, 223394_AT |
| GOTERM_{—} | 205891_AT, 203140_AT, 212171_X_AT, 213503_X_AT, 217757_AT, 205463_S_AT, 208816_X_AT, |
| BP_ALL | 1405_I_AT, 204655_AT, 210513_S_AT, 210427_X_AT, 201590_X_AT, 229830_AT, 1552914_A_AT, |
| | 204035_AT, 203508_AT, 210512_S_AT, 1555759_A_AT, 202859_X_AT, 211527_X_AT, 209827_S_AT, |
| | 203939_AT, 224859_AT, 233220_AT |
| GOTERM_{—} | 201464_X_AT, 210495_X_AT, 223723_AT, 201465_S_AT, 37152_AT, 210136_AT, 212298_AT, |
| BP_ALL | 209555_S_AT, 202435_S_AT, 227948_AT, 211924_S_AT, 203074_AT, 241938_AT, 202434_S_AT, |
| | 206488_S_AT, 214255_AT, 205067_AT, 202284_S_AT, 236561_AT, 200878_AT, 201925_S_AT, |
| | 201170_S_AT, 203729_AT, 217173_S_AT, 216017_S_AT, 205891_AT, 203665_AT, 202436_S_AT, |
| | 208816_X_AT, 201565_S_AT, 239519_AT, 201590_X_AT, 201466_S_AT, 223502_S_AT, 215602_AT, |
| | 203887_S_AT, 228766_AT, 210845_S_AT, 241773_AT, 201005_AT, 201324_AT, 212464_S_AT, |
| | 214211_AT, 237252_AT, 216442_X_AT, 213503_X_AT, 212171_X_AT, 39402_AT, 241722_X_AT, |
| | 1405_I_AT, 201566_X_AT, 210427_X_AT, 214866_AT, 211719_X_AT, 226218_AT, 223501_AT, |
| | 204661_AT, 202191_S_AT, 34210_AT, 200748_S_AT, 205099_S_AT, 204401_AT, 206835_AT, |
| | 200921_S_AT, 235944_AT, 203548_S_AT, 210512_S_AT, 200920_S_AT, 208436_S_AT, 211527_X_AT, |
| | 1562467_AT, 209906_AT, 1554992_AT, 203140_AT, 213281_AT, 204655_AT, 202437_S_AT, |
| | 210513_S_AT, 202068_S_AT, 1565754_X_AT, 238669_AT, 212372_AT, 203888_AT, 1565752_AT, |
| | 239451_AT, 1556583_A_AT, 219257_S_AT, 212803_AT, 1555759_A_AT, 241929_AT, 202067_S_AT, |
| | 215688_AT, 205098_AT, 223394_AT |
| GOTERM_{—} | 160020_AT, 210495_X_AT, 216442_X_AT, 201069_AT, 212171_X_AT, 213503_X_AT, 210427_X_AT, |
| CC_ALL | 211719_X_AT, 221731_X_AT, 202828_S_AT, 235944_AT, 210512_S_AT, 202827_S_AT, 211527_X_AT, |
| | 204475_AT, 201150_S_AT, 201148_S_AT, 213428_S_AT, 204359_AT, 204620_S_AT, 229004_AT, |
| | 208816_X_AT, 201149_S_AT, 210513_S_AT, 201147_S_AT, 215646_S_AT, 204619_S_AT, 201590_X_AT, |
| | 202756_S_AT, 228873_AT, 216546_S_AT, 217279_X_AT, 211571_S_AT, 212464_S_AT |
| GOTERM_{—} | 201464_X_AT, 160020_AT, 201465_S_AT, 229221_AT, 201069_AT, 212171_X_AT, 213503_X_AT, |
| BP_ALL | 212298_AT, 214297_AT, 39402_AT, 205463_S_AT, 221841_S_AT, 212014_X_AT, 210427_X_AT, |
| | 201473_AT, 222877_AT, 210916_S_AT, 229830_AT, 1557905_S_AT, 202828_S_AT, 1566901_AT, |
| | 205067_AT, 210512_S_AT, 202827_S_AT, 202859_X_AT, 211527_X_AT, 200878_AT, 204489_S_AT, |
| | 203665_AT, 204736_S_AT, 204490_S_AT, 220266_S_AT, 213281_AT, 212099_AT, 208816_X_AT, |
| | 210513_S_AT, 239519_AT, 209835_X_AT, 201590_X_AT, 204653_AT, 201466_S_AT, 212372_AT, |
| | 204035_AT, 203935_AT, 208937_S_AT, 217279_X_AT, 201005_AT |
| GOTERM_{—} | 202540_S_AT, 160020_AT, 229221_AT, 210495_X_AT, 216442_X_AT, 37152_AT, 212298_AT, |
| BP_ALL | 216248_S_AT, 39402_AT, 204622_X_AT, 1405_I_AT, 212014_X_AT, 221731_X_AT, 211719_X_AT, |
| | 222877_AT, 210916_S_AT, 204268_AT, 235739_AT, 223220_S_AT, 1557905_S_AT, 200921_S_AT, |
| | 202828_S_AT, 205067_AT, 202859_X_AT, 202827_S_AT, 236561_AT, 200920_S_AT, 209827_S_AT, |
| | 204489_S_AT, 204490_S_AT, 204620_S_AT, 204655_AT, 239519_AT, 204619_S_AT, 215646_S_AT, |
| | 209835_X_AT, 212372_AT, 204035_AT, 203935_AT, 204105_S_AT, 208937_S_AT, 1555759_A_AT, |
| | 217279_X_AT, 211571_S_AT, 202539_S_AT, 212464_S_AT |
| GOTERM_{—} | 202540_S_AT, 160020_AT, 229221_AT, 210495_X_AT, 216442_X_AT, 37152_AT, 21229 [symbol] _AT |
| BP_ALL | 216248_S_AT, 39402_AT, 204622_X_AT, 1405_I_AT, 212014_X_AT, 221731_X_AT, 211719_X_AT, |
| | 222877_AT, 210916_S_AT, 204268_AT, 235739_AT, 223220_S_AT, 1557905_S_AT, 200921_S_AT, |
| | 202 [symbol] 28_S_AT, 205067_AT, 202 [symbol] 59_X_AT, 202 [symbol] 27_S_AT, 236561_AT, 200920_S_AT, 209827_S_AT, |
| | 204489_S_AT, 204490_S_AT, 204620_S_AT, 204655_AT, 239519_AT, 204615_S_AT, 215646_S_AT, |
| | 209835_X_AT, 212372_AT, 204035_AT, 203935_AT, 204105_S_AT, 208937_S_AT, 1555759_A_AT, |
| | 217279_X_AT, 211571_S_AT, 202539_S_AT, 212464_S_AT |
| GOTERM_{—} | 201464_X_AT, 201465_S_AT, 37152_AT, 209555_S_AT, 39402_AT, 1405_I_AT, 201566_X_AT, |
| BP_ALL | 201473_AT, 226218_AT, 204923_AT, 206488_S_AT, 200921_S_AT, 1566901_AT, 205067_AT, |
| | 203548_S_AT, 200920_S_AT, 15 [symbol] 9149_AT, 203751_X_AT, 2132 [symbol] 1_AT, 212099_AT, 204655_AT, |
| | 201565_S_AT, 201466_S_AT, 212143_S_AT, 1552914_A_AT, 203935_AT, 204105_S_AT, 203233_AT, |
| | 227697_AT, 228766_AT, 203752_AT, 219257_S_AT, 1555759_A_AT, 241929_AT, 224859_AT, |
| | 210095_S_AT |
| GOTERM_{—} | 202540_S_AT, 22 [symbol] 221_AT, 2270 [symbol] _AT, 223723_AT, 2122 [symbol] _AT, 20 [symbol] 55_S_AT, 212024_X_AT, 221731_X_AT, |
| CC_ALL | 20 [symbol] 23_S_AT, 212066_X_AT, 22091 [symbol] _S_AT, 207 [symbol] 74_AT, 239225_AT, 2244 [symbol] 0_S_AT, 202828_S_AT, 2142 [symbol] _AT, |
| | 242 [symbol] 71_AT, 23 [symbol] 1_AT, 230 [symbol] 60_AT, 236561_AT, 226545_AT, 201 [symbol] 25_S_AT, 203729_AT, 227173_S_AT, |
| | 2044 [symbol] _S_AT, 22 [symbol] 90_AT, 202610_S_AT, 205505_AT, 220307_AT, 204 [symbol] 20_S_AT, 212099_AT, 211307_S_AT, |
| | 21 [symbol] 0_S_AT, 232649_AT, 233577_AT, 203922_S_AT, 21 [symbol] _S_AT, 229 [symbol] 37_X_AT, 221 [symbol] 15_AT, 214 [symbol] 0_AT, |
| | 2270 [symbol] 2_AT, 1552 [symbol] _A_AT, 20210 [symbol] _AT, 213 [symbol] 62_S_AT, 212977_AT, [symbol] 02754_S_AT, 22 [symbol] 6_AT, 20350 [symbol] _AT, |
| | 210 [symbol] 5_S_AT, 20 [symbol] 033_S_AT, 201005_AT, 201242_S_AT, 233220_AT, 207 [symbol] _S_AT, 224674_AT, 2145 [symbol] _X_AT, |
| | 237252_AT, 160020_AT, 2142 [symbol] 7_AT, 224 [symbol] 67_AT, [symbol] 3125_AT, 210 [symbol] 33_AT, 22 [symbol] 10_AT, 20 [symbol] _AT, 214 [symbol] _AT, |
| | 223502_AT, 205534_AT, 34120_AT, 2050 [symbol] _S_AT, 205566_AT, 200994_S_AT, 1552630_A_AT, 1557905_S_AT, |
| | 22 [symbol] 754_AT, 209670_AT, 22 [symbol] 42_AT, 215 [symbol] 36_S_AT, 217 [symbol] 7 [symbol] _AT, 205717_A_AT, 217078_S_AT, 240076_AT, |
| | 209047_AT, 209906_AT, 20921 [symbol] _AT, 221 [symbol] 40_AT, 204619_S_AT, 215616_S_AT, 201626_AT, 2120 [symbol] 0_S_AT, |
| | 20 [symbol] 20_AT, 219926_AT, 20 [symbol] 121_S_AT, 21167 [symbol] _S_AT, 2352 [symbol] 5_AT, 204257_AT, 203233_AT, 224 [symbol] 9_AT, |
| | 204 [symbol] 05_S_AT, 20 [symbol] 81_S_AT, 201125_S_AT, 217279_X_AT, 20 [symbol] 677_S_AT, 23 [symbol] 762_AT, 201243_S_AT, 204912_AT, |
| | 238 [symbol] _AT, 211 [symbol] 24_S_AT, 222 [symbol] 77_AT, 203217_S_AT, 209019_X_AT, 212062_AT, 228083_AT, 204222_S_AT, |
| | 20 [symbol] 88_S_AT, 20 [symbol] 171_AT, 2 [symbol] 2954_AT, 22 [symbol] 640_AT, 20 [symbol] 9_AT, 205542_AT, 24 [symbol] _AT, 2042 [symbol] _AT, 205 [symbol] 91_AT, |
| | 2047 [symbol] _S_AT, 2 [symbol] 4490_S_AT, 23 [symbol] 9_AT, 219 [symbol] _S_AT, 20 [symbol] 35_X_AT, 208161_S_AT, 20 [symbol] 21_AT, 218856_AT, |
| | 204715_AT, 223502_S_AT, 225337_AT, 236345_AT, 2204 [symbol] 4_AT, 203 [symbol] 7_S_AT, 22 [symbol] _AT, 20 [symbol] 46_AT, 20 [symbol] 2_AT, |
| | 2287 [symbol] 2_AT, 241773_AT, 224 [symbol] _AT, 20 [symbol] 53_AT, 201324_AT, 201625_S_AT, 202 [symbol] _S_AT, 204774_AT, 227180_AT, |
| | 241722_X_AT, 22 [symbol] 70 [symbol] _AT, 226228_AT, 20 [symbol] 61_AT, 225 [symbol] 09_AT, 227107_AT, 204401_AT, 2116 [symbol] 1_X_AT, 222062_AT, |
| | 202 [symbol] 8_S_AT, 2269 [symbol] 1_AT, 21 [symbol] 85_AT, 20354 [symbol] _S_AT, 22 [symbol] 0 [symbol] 2_AT, 217552_X_AT, 202 [symbol] 27_S_AT, 205419_AT, |
| | [symbol] 71 [symbol] 0_AT, 202071_AT, 2108 [symbol] _S_AT, 22 [symbol] 90_AT, 235299_AT, 225325_AT, 2020 [symbol] _S_AT, 220311_AT, 205714_AT, |
| | 20 [symbol] _AT, 20 [symbol] 5_AT, 2237 [symbol] _AT, 2286 [symbol] _AT, 201995_AT, 15565 [symbol] 3_A_AT, 22 [symbol] 297_AT, 226302_AT, 241 [symbol] 28_AT |
| | 210173_AT, [symbol] 020 [symbol] 7_S_AT, 215434_AT, 211572_S_AT, 2050 [symbol] _AT |
| GOTERM_{—} | 20 [symbol] 4 [symbol] 4_X_AT, 201465_S_AT, 229221_AT, 227069_AT, 210495_X_AT, 212296_AT, 210136_AT, |
| BP_ALL | 212014_X_AT, 221731_X_AT, 201473_AT, 210916_S_AT, 201471_S_AT, 235739_AT, 204960_S_AT, |
| | 202828_S_AT, 242871_AT, 22 [symbol] 64 [symbol] _AT, 236561_AT, 230360_AT, 156 [symbol] 149_AT, 20 [symbol] 9_S_AT, |
| | 216017_S_AT, 208891_AT, 212099_AT, 204620_S_AT, 201565_S_AT, 232619_AT, 1555832_S_AT, |
| | 243556_AT, 204961_S_AT, 210095_S_AT, 233220_AT, 160020_AT, 212171_X_AT, 214297_AT, |
| | 21624 [symbol] _S_AT, 201565_X_AT, 211719_X_AT, 228564_AT, 202191_S_AT, 1557505_S_AT, 201751_X_AT, |
| | 155 [symbol] 92_AT, 213281_AT, 224606_AT, 215646_S_AT, 204619_S_AT, 210513_S_AT, 204105_S_AT, |
| | 203752_S_AT, 212803_AT, 217279_X_AT, 37152_AT, 221841_S_AT, 221 [symbol] 77_AT, 24193 [symbol] _AT, |
| | 211 [symbol] 2_S_AT, 1566901_AT, 204141_AT, 200678_AT, 2023 [symbol] _AT, 204736_S_AT, 204490_S_AT, |
| | 2049 [symbol] _S_AT, 239519_AT, 209835_X_AT, 212190_AT, 201466_S_AT, 212143_S_AT, 2204 [symbol] 4_AT, |
| | 212124_AT, 201324_AT, 201012_AT, 212464_S_AT, 216 [symbol] 42_X_AT, 204622_X_AT, 241722_X_AT, |
| | 227240_AT, 226218_AT, 208893_S_AT, 219 [symbol] 45_AT, 210512_S_AT, 202827_S_AT, 205419_AT, |
| | 211917_X_AT, 1562467_AT, 204465_S_AT, 203240_AT, 208 [symbol] 2_S_AT, 2202 [symbol] 6_S_AT, 220935_S_AT, |
| | 212372_AT, 201935_AT, 225797_AT, 210171_AT, 211571_S_AT, 203920_AT, 215 [symbol] _AT |
| GOTERM_{—} | 201464_X_AT, 201465_S_AT, 219028_AT, 212298_AT, 216834_AT, 242794_AT, 213763_AT, |
| BP_ALL | 206472_S_AT, 203882_AT, 206171_AT, 226389_S_AT, 202859_X_AT, 236561_AT, 205891_AT, |
| | 207610_S_AT, 239519_AT, 225126_AT, 201466_S_AT, 203104_AT, 225115_AT, 212977_AT, |
| | 223939_AT, 203508_AT, 225368_AT, 202988_S_AT, 208937_S_AT, 209392_AT, 241773_AT, |
| | 233220_AT, 201012_AT, 212171_AT, 205463_S_AT, 1405_I_AT, 235457_AT, 214724_AT, 226218_AT, |
| | 229830_AT, 205099_S_AT, 211661_X_AT, 202686_S_AT, 222062_AT, 219908_AT, 210512_S_AT, |
| | 228186_S_AT, 205419_AT, 211527_X_AT, 210839_S_AT, 244414_AT, 209906_AT, 242405_AT, |
| | 213281_AT, 221840_AT, 204655_AT, 210513_S_AT, 209189_AT, 214054_AT, 203037_S_AT, |
| | 220313_AT, 205718_AT, 225738_AT, 203935_AT, 210258_AT, 225097_AT, 219257_S_AT, |
| | 1555759_A_AT, 210173_AT, 225557_AT, 201125_S_AT, 20509 [symbol] _AT |
| GOTERM_{—} | 204912_AT, 209555_S_AT, 217757_AT, 210233_AT, 211676_S_AT, 226218_AT, 205099_S_AT, |
| MF_ALL | 203104_AT, 203935_AT, 2064 [symbol] _S_AT, 203233_AT, 222062_AT, 228766_AT, 203508_AT, 236361_AT, |
| | 241929_AT, 205098_AT |
| GOTERM_{—} | 237252.AT, 210495_X_AT, 216442_X_AT, 229221_AT, 37152_AT, 209555_S_AT, 39402_AT, |
| BP_ALL | 205463_S_AT, 211924_S_AT, 212014_X_AT, 214866_AT, 211719_X_AT, 203074_AT, 210916_S_AT, |
| | 229830_AT, 206488_S_AT, 1557905_S_AT, 208960_S_AT, 235944_AT, 205067_AT, 201150_S_AT, |
| | 204465_S_AT, 201148_S_AT, 204489_S_AT, 203665_AT, 204490_S_AT, 224606_AT, 201149_S_AT, |
| | 201147_S_AT, 209835_X_AT, 212372_AT, 203888_AT, 203887_S_AT, 1555832_S_AT, 228766_AT, |
| | 210845_S_AT, 241929_AT, 201005_AT, 208961_S_AT, 212464_S_AT |
| GOTERM_{—} | 202540_S_AT, 210495_X_AT, 229221_AT, 37152_AT, 212298_AT, 212014_X_AT, 211924_S_AT, |
| BP_ALL | 221731_X_AT, 222877_AT, 210916_S_AT, 204268_AT, 235739_AT, 223220_S_AT, 202828_S_AT, |
| | 205067_AT, 202859_X_AT, 236561_AT, 209827_S_AT, 204489_S_AT, 204490_S_AT, 204620_S_AT, |
| | 239519_AT, 209835_X_AT, 204035_AT, 208937_S_AT, 209392_AT, 210845_S_AT, 201005_AT, |
| | 202539_S_AT, 201012_AT, 212464_S_AT, 160020_AT, 216442_X_AT, 216248_S_AT, 39402_AT, |
| | 204622_X_AT, 1405_I_AT, 211719_X_AT, 214866_AT, 1557905_S_AT, 200921_S_AT, 202827_S_AT, |
| | 200920_S_AT, 210839_S_AT, 204655_AT, 215646_S_AT, 204619_S_AT, 203037_S_AT, 212372_AT, |
| | 203935_AT, 204105_S_AT, 1555759_A_AT, 217279_X_AT, 211571_S_AT |
| GOTERM_{—} | 210495_X_AT, 216442_X_AT, 210136_AT, 212171_X_AT, 212298_AT, 39402_AT, 227948_AT, |
| BP_ALL | 211719_X_AT, 202191_S_AT, 203940_S_AT, 200921_S_AT, 214255_AT, 205067_AT,210512_S_AT, |
| | 200920_S_AT, 211527_X_AT, 203665_AT, 205891_AT, 212099_AT, 239519_AT, 210513_S_AT, |
| | 1565754_X_AT, 212372_AT, 1565752_AT, 204105_S_AT, 215602_AT, 219257_S_AT, 208937_S_AT, |
| | 212464_S_AT |
| GOTERM_{—} | 201464_X_AT, 213 [symbol] 91_S_AT, 229221_AT, 201465_S_AT, 219028_AT, 212298_AT, 209555_S_AT, |
| BP_ALL | 212014_X_AT, 201473_AT, 211352_S_AT, 210916_S_AT, 201471_S_AT, 235739_AT, 234840_S_AT, |
| | 205067_AT, 217997_AT, 236561_AT, 202859_X_AT, 2022 [symbol] 4_S_AT, 209 [symbol] 27_S_AT, 201925_S_AT, |
| | 1569149_AT, 204489_S_AT, 212099_AT, 201147_S_AT, 201565_S_AT, 203753_AT, 207700_S_AT, |
| | 1552914_A_AT, 225215_AT, 1555832_S_AT, 228766_AT, 222146_S_AT, 205312_AT, 243556_AT, |
| | 20 [symbol] 961_S_AT, 210095_S_AT, 212171_X_AT, 216248_S_AT, 39402_AT, 205463_S_AT, 1405_I_AT, |
| | 235457_AT, 201566_X_AT, 22 [symbol] 964_AT, 223501_AT, 229 [symbol] 10_AT, 212387_AT, 202086_AT, |
| | 1557905_S_AT, 222670_S_AT, 200920_S_AT, 20 [symbol] 436_S_AT, 201150_S_AT, 203751_X_AT, 217996_AT, |
| | 20114 [symbol] _S_AT, 209906_AT, 1554992_AT, 2132 [symbol] 1_AT, 224606_AT, 210513_S_AT, 1565754_X_AT, |
| | 225842_AT, 238669_AT, 2353295_AT, 1565752_AT, 203233_AT, 204105_S_AT, 212386_AT, 225097_AT, |
| | 203752_S_AT, 219257_S_AT, 225557_AT, 37152_AT, 22794 [symbol] _AT, 221841_S_AT, 242794_AT, |
| | 21294 [symbol] _AT, 204923_AT, 201041_S_AT, 206488_S_AT, 213763_AT, 1554453_AT, 1566901_AT, |
| | 220066_AT, 20087 [symbol] _AT, 205891_AT, 203663_AT, 204490_S_AT, 201149_S_AT, 239519_AT, |
| | 209835_X_AT, 225116_AT, 204715_AT, 206157_AT, 201466_S_AT, 223502_S_AT, 212143_S_AT, |
| | 204035_AT, 215602_AT, 212124_AT, 225368_AT, 241273_AT, 224859_AT, 218501_AT, 243797_AT, |
| | 204622_X_AT, 227240_AT, 22621 [symbol] _AT, 218559_S_AT, 227107_AT, 204401_AT, 222062_AT, |
| | 200921_S_AT, 20354 [symbol] _S_AT, 217552_X_AT, 210512_S_AT, 211527_X_AT, 244414_AT, 203140_AT, |
| | 242405_AT, 220266_S_AT, 204655_AT, 1552553_A_AT, 2091 [symbol] 9_AT, 201044_X_AT, 203935_AT, |
| | 227697_AT, 1555759_A_AT, 241929_AT, 215688_AT, 223394_AT |
| GOTERM_{—} | 201464_X_AT, 201465_S_AT, 37152_AT, 20 [symbol] 555_S_AT, 1405_I_AT, 201565_X_AT, 201473_AT, |
| BP_ALL | 226218_AT, 204923_AT, 206488_S_AT, 200921_S_AT, 1566901_AT, 203548_S_AT, 200920_S_AT, |
| | 203751_X_AT, 1569149_AT, 213281_AT, 204655_AT, 201565_S_AT, 201466_S_AT, 212143_S_AT, |
| | 1552914_A_AT, 203935_AT, 203233_AT, 204205_S_AT, 227697_AT, 22 [symbol] 76 [symbol] _AT, 203752_S_AT, |
| | 1555759_A_AT, 241929_AT, 224859_AT, 210095_S_AT |
| GOTERM_{—} | 201464_X_AT, 201465_S_AT, 229221_AT, 227069_AT, 210495_X_AT, 212298_AT, 210136_AT, |
| BP_ALL | 212014_X_AT, 221731_X_AT, 201473_AT, 210916_S_AT, 201471_S_AT, 235739_AT, 208960_S_AT, |
| | 202828_S_AT, 242 [symbol] 71_AT, 22 [symbol] 648;_AT, 236561_AT, 230360_AT, 1569149_AT, 204489_S_AT, |
| | 216017_S_AT, 208 [symbol] 91_AT, 212099_AT, 204620_S_AT, 201565_S_AT, 232649_AT, 1555 [symbol] 32_S_AT, |
| | 201005_AT, 243556_AT, 208961_S_AT, 210095_S_AT, 233220_AT, 160020_AT, 214297_AT, |
| | 212171_X_AT, 216248_S_AT, 201566_X_AT, 211719_X_AT, 228964_AT, 202193_S_AT, 1557905_S_AT, |
| | 203751_X_AT, 1554992_AT, 213281_AT, 224606_AT, 215646_S_AT, 204619_S_AT, 210513_S_AT, |
| | 204105_S_AT, 203752_S_AT, 212803_AT, 217279_X_AT, 37152_AT, 221 [symbol] 41_AT, 222 [symbol] 77_AT, |
| | 24193 [symbol] _AT, 211962_S_AT, 1566901_AT, 204141_AT, 200878_AT, 2025 [symbol] _AT, 204736_S_AT, |
| | 204490_S_AT, 2049 [symbol] _S_AT, 239519_AT, 209835_X_AT, 212190_AT, 201466_S_AT, 212143_S_AT, |
| | 220484_AT, 212124_AT, 201324_AT, 201012_AT, 212464_S_AT, 216 [symbol] 42_X_AT, 204622_X_AT, |
| | 241722_X_AT, 227240_AT, 22621 [symbol] _AT, 20889 [symbol] _S_AT, 219 [symbol] 45_AT, 210512_S_AT, 202 [symbol] 27_S_AT, |
| | 205419_AT, 211527_X_AT, 1562467_AT, 2044 [symbol] 5_S_AT, 203140_AT, 208 [symbol] 92_S_AT, 220266_S_AT, |
| | 220935_S_AT, 212372_AT, 203935_AT, 229797_AT, 210173_AT, 211571_S_AT, 203320_AT, 215 [symbol] _AT |
| GOTERM_{—} | 201464_X_AT, 201465_S_AT, 37152_AT, 212298_AT, 210136_AT, 209555_S_AT, 221841_S_AT, |
| BP_ALL | 1405_I_AT, 201566_X_AT, 201473_AT, 226218_AT, 218559_S_AT, 204923_AT, 206488_S_AT, |
| | 200921_S_AT, 1566901_AT, 203548_S_AT, 236561_AT, 222670_S_AT, 200920_S_AT, 1569149_AT, |
| | 203751_X_AT, 203140_AT, 220266_S_AT, 20499 [symbol] _S_AT, 220935_S_AT, 213281_AT, 204655_AT, |
| | 201565_S_{——}AT, 239519_AT, 201466_S_AT, 1552914_A_AT, 212143_S_AT, 2 [symbol] 3935_AT, 203233_AT, |
| | 204105_S_AT, 227697_AT, 228766_AT, 203752_AT, 1555759_A_AT, 205312_AT, 241929_AT, |
| | 224859_AT, 210095_S_AT |
| GOTERM_{—} | 202540_S_AT, 202464_X_AT, 21 [symbol] 1_S_AT, 2014 [symbol] 5_S_AT, 223724_AT, 227069_AT, 21902 [symbol] _AT, 2122 [symbol] _AT, 212 [symbol] 14_X_AT, |
| BP_ALL | 216 [symbol] 34_AT, 22 [symbol] 412_AT, 20 [symbol] 60_S_AT, 217 [symbol] 97_AT, 205067_AT, 20 [symbol] _X_AT, 201170_S_AT, 20 [symbol] 729_AT, 21712 [symbol] _S_AT, |
| | 2044 [symbol] _S_AT, 216017_S_AT, 225171_AT, 220 [symbol] 07_AT, 202 [symbol] _S_AT, 201147_S_AT, 201 [symbol] 0_X_AT, 230266_AT, 20 [symbol] 653_AT, |
| | 221815_AT, 1552914_A_AT, 207700_S_AT, 202104_AT, 1555 [symbol] 2_S_AT, 204 [symbol] _AT, 222144_S_AT, 2287 [symbol] _AT, 2100 [symbol] 5_S_AT, |
| | 214211_AT, 2145 [symbol] 1_X_AT, 2162 [symbol] _S_AT, 6 [symbol] 25_AT, 205 [symbol] 3_S_AT, 140 [symbol] _I_AT, 2015 [symbol] _X_AT, 2 [symbol] 4_AT, 21171 [symbol] _ [symbol] _AT, |
| | 22 [symbol] 03_AT, 2020 [symbol] _AT, 2173 [symbol] 7_AT, 22 [symbol] 30_AT, 205099_S_AT, 1557905_S_AT, 21 [symbol] 02_S_AT, 20 [symbol] 5_AT, 235 [symbol] 4_AT, |
| | 222 [symbol] 70_S_AT, 200 [symbol] 20_S_AT, 2191 [symbol] 3_S_AT, 20 [symbol] _S_AT, 2278 [symbol] _AT, 2087 [symbol] _X_AT, 201150_S_AT, 20114 [symbol] _S_AT, 1554 [symbol] 2_AT, |
| | 217757_AT, 2132 [symbol] 1_AT, 15 [symbol] 5754_X_AT, 2295 [symbol] _AT, 20 [symbol] 0 [symbol] _AT, 211 [symbol] _S_AT, 223 [symbol] 42_AT, 2257 [symbol] _AT, 214250_S_AT, |
| | 20 [symbol] _AT, 23 [symbol] _AT, 154 [symbol] 752_AT, 2032 [symbol] _AT, 20420 [symbol] _S_AT, 3702 [symbol] _AT, 20 [symbol] 7 [symbol] 2_S_AT, 225557_AT, 2173 [symbol] 2_AT, [symbol] 7152_AT, |
| | 227943_AT, 242794_AT, 242 [symbol] 34_AT, 201041_S_AT, 204 [symbol] 23_AT, 202434_S_AT, 20 [symbol] _S_AT, 212562_S_AT, 2251 [symbol] _AT, |
| | 2200 [symbol] _AT, 200 [symbol] 78_AT, 225144_AT, 202 [symbol] _AT, 205 [symbol] 1_AT, 2 [symbol] 65_AT, 204734_S_AT, 20 [symbol] _X_AT, 2 [symbol] 1 [symbol] _AT, 20 [symbol] _X_AT, |
| | 2 [symbol] _AT, 225116_AT, 212180_AT, 20 [symbol] 715_AT, 20 [symbol] 4 [symbol] _S_AT, 22 [symbol] 502_S_AT, 21214 [symbol] _S_AT, 235602_AT, 22 [symbol] _AT, |
| | 2 [symbol] 67_S_AT, 202 [symbol] _S_AT, 208 [symbol] 7_S_AT, 222 [symbol] 1_S_AT, 2025 [symbol] _S_AT, 21 [symbol] 501_AT, 2 [symbol] 7 [symbol] 7_AT, 2272 [symbol] 0_AT, 210427_X_AT, |
| | 22 [symbol] 21 [symbol] _AT, 21 [symbol] _S_AT, 204 [symbol] 61_AT, 200 [symbol] _S_AT, 204401_AT, 20 [symbol] _S_AT, 2220 [symbol] 2_AT, 22 [symbol] _AT, 227 [symbol] _AT, 21 [symbol] _AT, |
| | 220512_S_AT, 225 [symbol] _AT, 217552_X_AT, 212527_X_AT, 202071_AT, 244424_AT, 203 [symbol] 40_AT, 20 [symbol] 52_S_AT, 220 [symbol] _S_AT, |
| | 242405_AT, 202241_AT, 201044_X_AT, 220313_AT, 20 [symbol] _AT, 2102 [symbol] _AT, 155636 [symbol] _A_AT, 201 [symbol] _AT, 22 [symbol] 302_AT, 215 [symbol] _AT, |
| | 2050 [symbol] _AT, 210 [symbol] _X_AT, 229225_AT, 210124_AT, 2 [symbol] 5_S_AT, 229155_AT, 202 [symbol] _S_AT, 221011_S_AT, 20247 [symbol] _AT, |
| | 20247 [symbol] _AT, 20 [symbol] 074_AT, 210916_S_AT, 211 [symbol] 2_X_AT, 1555 [symbol] 22_AT, 201471_S_AT, 2357 [symbol] _AT, 204472_S_AT, 200 [symbol] 2_AT, |
| | 226 [symbol] _S_AT, 2244 [symbol] _S_AT, 202 [symbol] _S_AT, 214255_AT, 202 [symbol] _S_AT, 2 [symbol] 1_AT, 20 [symbol] 27_A_AT, 201525_S_AT, 15 [symbol] 49_AT, |
| | 22 [symbol] 90_AT, 207610_S_AT, 20 [symbol] 91_AT, 2120 [symbol] _AT, 20 [symbol] 75 [symbol] _AT, 1 [symbol] 2_A_AT, 225125_AT, 212 [symbol] 77_AT, 201 [symbol] _AT, 20 [symbol] 312_AT, |
| | 230 [symbol] 25_AT, 2 [symbol] 0 [symbol] 45_S_AT, 24 [symbol] _AT, 201005_AT, 20 [symbol] 1_S_AT, 2 [symbol] 20_AT, 2132 [symbol] _S_AT, 1500 [symbol] _AT, 227252_AT, |
| | 212171_X_AT, 2142 [symbol] 7_AT, 39402_AT, 21023 [symbol] _AT, 2 [symbol] 457_AT, 214 [symbol] 66_AT, 23 [symbol] _AT, 2021 [symbol] 1_S_AT, 34210_AT, 2 [symbol] 4_AT, |
| | 205566_AT, 2 [symbol] 1 [symbol] _X_AT, 221463_AT, 20 [symbol] 436_S_AT, 20 [symbol] 06_AT, 20276 [symbol] _AT, 22 [symbol] 606_AT, 210513_S_AT, 2352 [symbol] 5_AT, 2 [symbol] 451_AT, |
| | 200 [symbol] 72_AT, 212 [symbol] 5_AT, 22 [symbol] 0 [symbol] 7_AT, 21 [symbol] 257_S_AT, 21 [symbol] 4_AT, 222 [symbol] 03_AT, 21727 [symbol] _X_AT, 221 [symbol] 41_S_AT, 234 [symbol] _AT, |
| | 211 [symbol] 24_AT, 2242 [symbol] _S_AT, 212 [symbol] _AT, 21 [symbol] 7 [symbol] _AT, 240 [symbol] 6_AT, 204271_AT, 1554451_AT, 1555 [symbol] 01_AT, 1550 [symbol] _AT, |
| | 20101 [symbol] _AT, 20 [symbol] _AT, 2024 [symbol] _S_AT, 2044 [symbol] 0_S_AT, 20 [symbol] _S_AT, 20114 [symbol] _S_AT, 236570_AT, 20 [symbol] 257_AT, 22 [symbol] 37_AT, |
| | 2040 [symbol] 5_AT, 212224_AT, 2253 [symbol] _AT, 200 [symbol] 7_AT, 24177 [symbol] _AT, 224 [symbol] _AT, 201324_AT, 200 [symbol] 1_AT, 201012_AT, 212 [symbol] 4_S_AT, |
| | 216 [symbol] 2_X_AT, 21 [symbol] 50 [symbol] _X_AT, 204622_X_AT, 242722_X_AT, 22 [symbol] 70 [symbol] _AT, 21 [symbol] _S_AT, 227107_AT, 221 [symbol] 1_X_AT, 20 [symbol] 0_S_AT, |
| | 20 [symbol] 1_S_AT, 225207_AT, 200 [symbol] 25_S_AT, 20 [symbol] _S_AT, 22 [symbol] _AT, 202 [symbol] 27_S_AT, 20042 [symbol] _AT, [symbol] 7200_AT, [symbol] 0039_S_AT, |
| | 1542467_AT, 206571_S_AT, 22 [symbol] _AT, 224702_AT, 220935_S_AT, 204 [symbol] 5_AT, 15 [symbol] 2553_A_AT, 202437_S_AT, 20 [symbol] 1 [symbol] _AT, |
| | 20 [symbol] _S_AT, 2140 [symbol] _AT, 20 [symbol] 0 [symbol] 7_S_AT, 212 [symbol] 72_AT, 203 [symbol] _AT, 217272_S_AT, 227 [symbol] 7_AT, 15557 [symbol] _A_AT, 241929_AT, |
| | 2020 [symbol] 7_S_AT, 21943 [symbol] _AT, 20 [symbol] 20_AT, 222 [symbol] 4_AT |
| GOTERM_{—} | 201464_X_AT, 237252_AT, 201465_S_AT, 39402_AT, 1405_I_AT, 202708_S_AT, 218934_S_AT, |
| BP_ALL | 202086_AT, 211661_X_AT, 222062_AT, 203882_AT, 239818_X_AT, 205067_AT, 220066_AT, |
| | 208436_S_AT, 202014_AT, 214453_S_AT, 202241_AT, 213281_AT, 204655_AT, 1552553_A_AT, |
| | 209189_AT, 211676_S_AT, 229937_X_AT, 201466_S_AT, 206157_AT, 203888_AT, 227697_AT, |
| | 203887_S_AT, 37028_AT, 205495_S_AT, 1555759_A_AT, 208438_S_AT, 213293_S_AT |
| GOTERM_{—} | 202540_S_AT, 229221_AT, 227069_AT, 223723_AT, 212298_AT, 209555_S_AT, 212014_X_AT, |
| CC_ALL | 221731_X_AT, 203923_S_AT, 211066_X_AT, 210916_S_AT, 207674_AT, 219225_AT, 224480_S_AT, |
| | 202828_S_AT, 214255_AT, 242871_AT, 238681_AT, 230360_AT, 236561_AT, 201925_S_AT, |
| | 203729_AT, 217173_S_AT, 204489_S_AT, 228450_AT, 207610_S_AT, 205505_AT, 220307_AT, |
| | 204620_S_AT, 211307_S_AT, 216080_S_AT, 232649_AT, 213577_AT, 203922_S_AT, 218686_S_AT, |
| | 229937_X_AT, 214830_AT, 221815_AT, 227052_AT, 1552914_A_AT, 203104_AT, 213562_S_AT, |
| | 212977_AT, 202756_S_AT, 228766_AT, 203508_AT, 210845_S_AT, 206033_S_AT, 201005_AT, |
| | 201242_S_AT, 233220_AT, 207819_S_AT, 224674_AT, 214581_X_AT, 237252_AT, 160020_AT, |
| | 214297_AT, 224967_AT, 63825_AT, 210233_AT, 228910_AT, 209949_AT, 214866_AT, 223501_AT, |
| | 205534_AT, 34210_AT, 205099_S_AT, 205566_AT, 209994_S_AT, 1552690_A_AT, 1557905_S_AT, |
| | 228754_AT, 209670_AT, 215836_S_AT, 217678_AT, 205717_X_AT, 217078_S_AT, 240076_AT, |
| | 209047_AT, 209906_AT, 209218_AT, 221840_AT, 204619_S_AT, 215646_S_AT, 201626_AT, |
| | 211030_S_AT, 205920_AT, 219926_AT, 208121_S_AT, 211676_S_AT, 235295_AT, 204257_AT, |
| | 203233_AT, 224989_AT, 204105_S_AT, 204881_S_AT, 201125_S_AT, 217279_X_AT, 201627_S_AT, |
| | 239791_AT, 201243_S_AT, 204912_AT, 23 [symbol] 638_AT, 211924_S_AT, 222877_AT, 203217_S_AT, |
| | 209079_X_AT, 212062_AT, 22 [symbol] 083_AT, 204222_S_AT, 206488_S_AT, 206171_AT, 241954_AT, |
| | 228640_AT, 205542_AT, 243894_AT, 204359_AT, 205891_AT, 204736_S_AT, 204490_S_AT, |
| | 239519_AT, 219386_S_AT, 209835_X_AT, 208161_S_AT, 209921_AT, 218856_AT, 204715_AT, |
| | 223502_S_AT, 225337_AT, 236345_AT, 220484_AT, 203387_S_AT, 223939_AT, 209146_AT, |
| | 209392_AT, 228762_AT, 241773_AT, 224859_AT, 209993_AT, 201324_AT, 201625_S_AT, |
| | 202539_S_AT, 204774_AT, 227180_AT, 241722_X_AT, 226218_AT, 204661_AT, 225809_AT, |
| | 227107_AT, 20 [symbol] 401_AT, 211661_X_AT, 222062_AT, 202686_S_AT, 226931_AT, 219385_AT, |
| | 223092_AT, 217552_X_AT, 202827_S_AT, 205419_AT, 87100_AT, 202071_AT, 210839_S_AT, |
| | 224990_AT, 235299_AT, 225325_AT, 202068_S_AT, 220313_AT, 205718_AT, 203888_AT, 203935_AT, |
| | 223798_AT, 228696_AT, 201995_AT, 1556583_S_AT, 229797_AT, 226302_AT, 241929_AT, 210173_AT, |
| | 202067_S_AT, 219434_AT, 211571_S_AT, 205098_AT |
| GOTERM_{—} | 21 [symbol] _S_AT, 201464_X_AT, 2 [symbol] 540_X_AT, 2270 [symbol] _AT, 2 [symbol] 43_S_AT, 21 [symbol] 0 [symbol] _AT, 2122 [symbol] _AT, 212014_X_AT, 214 [symbol] 3 [symbol] _AT, |
| BP_ALL | 21 [symbol] 412_AT, 20 [symbol] 40_S_AT, 217997_AT, 2 [symbol] [symbol] 0 [symbol] 7_AT, 20 [symbol] _X_AT, 201270_S_AT, 20 [symbol] 7 [symbol] _AT, 2 [symbol] _S_AT, 21 [symbol] 17_S_AT, |
| | 235171_AT, 220307_AT, 201 [symbol] _S_AT, 20114 [symbol] _S_AT, 201 [symbol] _X_AT, 230 [symbol] _AT, 204 [symbol] _AT, 22 [symbol] 15_AT, 155 [symbol] 14_A_AT, |
| | 20 [symbol] 00_S_AT, 20 [symbol] 04_AT, 1555 [symbol] 32_S_AT, 20 [symbol] _AT, 2221 [symbol] _I_AT, 22 [symbol] 7 [symbol] _AT, 2 [symbol] 5_S_AT, 214211_AT, 2145 [symbol] 1_X_AT, |
| | 21 [symbol] _ [symbol] _AT, [symbol] 5_AT, 20 [symbol] _S_AT, 1405_I_AT, 201544_X_AT, 22 [symbol] _AT, 2117 [symbol] _X_AT, 22 [symbol] 01_AT, 20 [symbol] _AT, 212 [symbol] 7_AT, |
| | 22 [symbol] 0_AT, 20 [symbol] _S_AT, 1557 [symbol] 5_S_AT, 2 [symbol] 02_S_AT, 20 [symbol] 5_AT, 222 [symbol] 70_S_AT, 2 [symbol] 20_S_AT, 21 [symbol] _S_AT, 2 [symbol] _S_AT, |
| | 217 [symbol] 6_AT, 20 [symbol] 751_X_AT, 201150_S_AT, 2011 [symbol] _S_AT, 155 [symbol] 2_AT, 217757_AT, 21 [symbol] _AT, 1545734_X_AT, 22 [symbol] _AT, |
| | 20 [symbol] _AT, 211 [symbol] 4_S_AT, 22 [symbol] 2_AT, 22 [symbol] _AT, 21 [symbol] 0_S_AT, 2020 [symbol] _AT, 23 [symbol] _AT, 15 [symbol] 5752_AT, 20 [symbol] _AT, |
| | 204 [symbol] _S_AT, 370 [symbol] _AT, 2 [symbol] 5 [symbol] _S_AT, 225557_AT, 237 [symbol] 1_AT, 37152_AT, 22 [symbol] _AT, 2427 [symbol] _AT, [symbol] _AT, 20 [symbol] 0 [symbol] _S_AT, |
| | 20 [symbol] 3_AT, 20 [symbol] _S_AT, 21 [symbol] 42_S_AT, 22517 [symbol] _AT, 22 [symbol] 6_AT, 200 [symbol] 78_AT, 225366_AT, 202 [symbol] _AT, 2 [symbol] 3665_AT, 2047 [symbol] 6_S_AT, |
| | 20 [symbol] 1_AT, 20 [symbol] _X_AT, 23 [symbol] 519_AT, 20 [symbol] _X_AT, 21 [symbol] 3 [symbol] _AT, 22 [symbol] 11 [symbol] _AT, 2121 [symbol] 0_AT, 20 [symbol] 71 [symbol] _AT, 22 [symbol] _S_AT, |
| | 201 [symbol] _S_AT, 212143_S_AT, 215 [symbol] 02_AT, 22 [symbol] _AT, 2 [symbol] _S_AT, 20 [symbol] 7_S_AT, 2276 [symbol] 1_S_AT, 202 [symbol] _S_AT, 21 [symbol] _AT, |
| | 24 [symbol] 7 [symbol] 7_AT, 2273 [symbol] 0_AT, 210427_X_AT, 22 [symbol] _AT, 21 [symbol] _S_AT, 2007 [symbol] _S_AT, 204401_AT, [symbol] 0 [symbol] _S_AT, 222062_AT, |
| | 21 [symbol] 45_AT, 2272 [symbol] _AT, 21 [symbol] _AT, 210522_S_AT, 22 [symbol] 1 [symbol] _AT, 217 [symbol] 2_X_AT, 2 [symbol] 525_X_AT, 2020 [symbol] 1_AT, 2 [symbol] 1 [symbol] _AT, 20 [symbol] 0_AT, |
| | 20 [symbol] 02_S_AT, 22 [symbol] 4_S_AT, 2424 [symbol] _AT, 202241_AT, 2 [symbol] 1044_X_AT, 220 [symbol] 13_AT, 2 [symbol] _AT, 210 [symbol] _AT, 1 [symbol] 1_A_AT, |
| | 20 [symbol] 5_AT, 21 [symbol] 1_AT, 215 [symbol] _AT, 20 [symbol] 0 [symbol] _AT, 210 [symbol] 5_X_AT, 22 [symbol] 221_AT, 210 [symbol] 6_AT, 20 [symbol] 5_S_AT, 23 [symbol] 15 [symbol] _AT, 222041_S_AT, |
| | 201473_AT, [symbol] _AT, 21 [symbol] 1 [symbol] _S_AT, 2113 [symbol] 2_S_AT, 1 [symbol] 17_AT, 201471_S_AT, 2 [symbol] 57 [symbol] _AT, 204472_S_AT, 20 [symbol] 2_AT, |
| | 22 [symbol] _S_AT, 2244 [symbol] 0_S_AT, 202 [symbol] _S_AT, 21425 [symbol] _AT, 2022 [symbol] 4_S_AT, 23 [symbol] 1_AT, 20 [symbol] 27_S_AT, 20 [symbol] 5_S_AT, 15 [symbol] _AT, |
| | 22 [symbol] 0_AT, 207 [symbol] 10_S_AT, 20 [symbol] _AT, 212 [symbol] _AT, 20 [symbol] 7 [symbol] _AT, 155 [symbol] 1_A_AT, 225215_AT, 212477_AT, 20 [symbol] 0 [symbol] _AT, 20 [symbol] 22_AT, |
| | 220 [symbol] 25_AT, 210 [symbol] 5_S_AT, 24 [symbol] 6_AT, 201005_AT, 20 [symbol] 3_S_AT, 2 [symbol] 220_AT, 215292_S_AT, 1500 [symbol] _AT, 212171_X_AT, |
| | 2142 [symbol] 7_AT, [symbol] 402_AT, 2 [symbol] 2 [symbol] _AT, 2 [symbol] 4 [symbol] 7_AT, 224 [symbol] _AT, 2 [symbol] 0 [symbol] _AT, 202191_S_AT, 223 [symbol] _AT, 202 [symbol] _AT, 2 [symbol] 1 [symbol] _X_AT, |
| | 223 [symbol] _AT, 20 [symbol] _S_AT, 20 [symbol] 04_AT, 2027 [symbol] _AT, 22 [symbol] 0 [symbol] _AT, 220 [symbol] 1 [symbol] _S_AT, 215 [symbol] _AT, 23 [symbol] 1_AT, 200 [symbol] 72_AT, 212 [symbol] _AT, |
| | 22 [symbol] 7_AT, 22 [symbol] 7_S_AT, 21 [symbol] 4_AT, 212 [symbol] 0 [symbol] _AT, 217279_X_AT, 221841_S_AT, 2 [symbol] _AT, 2 [symbol] 34_S_AT, 23422 [symbol] _S_AT, |
| | 212 [symbol] _AT, 21 [symbol] 2_AT, 24 [symbol] _AT, 204172_AT, 25 [symbol] _AT, 15 [symbol] 01_AT, 25 [symbol] _AT, 200 [symbol] 1 [symbol] _AT, 203 [symbol] _AT, 204 [symbol] 0_S_AT, |
| | 20 [symbol] _S_AT, 2033 [symbol] _S_AT, 2 [symbol] 30_AT, 20 [symbol] 157_AT, 225 [symbol] 7_AT, 20 [symbol] 015_AT, 232224_AT, 225 [symbol] _AT, 24177 [symbol] _AT, 2 [symbol] 2_AT, |
| | 22 [symbol] 50_AT, 2010 [symbol] 2_AT, 20 [symbol] 3_AT, 212 [symbol] _ [symbol] _AT, 2 [symbol] 4442_X_AT, 21 [symbol] _X_AT, 20 [symbol] 22_X_AT, 2 [symbol] 1722_X_AT, 22 [symbol] 708_AT, |
| | 22 [symbol] _S_AT, 227107_AT, 211 [symbol] 2_X_AT, 20 [symbol] 0_S_AT, 20 [symbol] S_AT, 225207_AT, 20 [symbol] 21_S_AT, [symbol] 0 [symbol] _S_AT, 2230 [symbol] 2_AT, |
| | 202 [symbol] 7_S_AT, 205419_AT, [symbol] 7300_AT, 1562 [symbol] 7_AT, 210 [symbol] _S_AT, 20 [symbol] 71_S_AT, 22 [symbol] _AT, 22470 [symbol] _AT, 220 [symbol] 5_S_AT, |
| | 20 [symbol] _AT, 15525 [symbol] _A_AT, 20 [symbol] _AT, 21 [symbol] 4_AT, 20 [symbol] 097_S_AT, 21237 [symbol] _AT, 217 [symbol] _X_AT, 227 [symbol] 7_AT, 15 [symbol] 5258_X_AT, |
| | 241 [symbol] _AT, 21 [symbol] 4 [symbol] 4_AT, 2 [symbol] _AT, 22 [symbol] _AT |
| GOTERM_{—} | 39402_AT, 217757_AT, 216243_S_AT, 1405_I_AT, 204655_AT, 2091 [symbol] 9_AT, 201473_AT, 201044_X_AT, |
| BP_ALL | 201041_S_AT, 23 [symbol] 669_AT, 212657_S_AT, 205067_AT, 202284_S_AT, 1555759_A_AT, 207 [symbol] 19_S_AT |
| GOTERM_{—} | 201464_X_AT, 201465_S_AT, 229221_AT, 212171_X_AT, 39402_AT, 1405_I_AT, 201566_X_AT, |
| BP_ALL | 212014_X_AT, 226218_AT, 223501_AT, 210916_S_AT, 218559_S_AT, 204923_AT, 1557905_S_AT, |
| | 222067_AT, 205067_AT, 210512_S_AT, 202284_S_AT, 222670_S_AT, 217552_X_AT, 220066_AT, |
| | 211527_X_AT, 201925_S_AT, 209906_AT, 204489_S_AT, 203665_AT, 205 [symbol] 91_AT, 203140_AT, |
| | 204490_S_AT, 227752_AT, 213281_AT, 204655_AT, 210513_S_AT, 201565_S_AT, 209635_X_AT, |
| | 221502_S_AT, 201466_S_AT, 1552914_A_AT, 203233_AT, 205312_AT, 1555759_A_AT, 224859_AT |
| GOTERM_{—} | 39402_AT, 217757_AT, 216243_S_AT, 1405_I_AT, 204655_AT, 2091 [symbol] 9_AT, 201473_AT, 201044_X_AT, |
| BP_ALL | 201041_S_AT, 23 [symbol] 669_AT, 212657_S_AT, 205067_AT, 2022 [symbol] 4_S_AT, 1555759_A_AT, 207 [symbol] 19_S_AT |
| GOTERM_{—} | 202540_S_AT, 2014 [symbol] 4_X_AT, 201465_S_AT, 21 [symbol] 02 [symbol] _AT, 216248_S_AT, 39402_AT, 204622_X_AT, |
| BP_ALL | 205463_S_AT, 1405_I_AT, 211924_S_AT, 214 [symbol] _AT, 223877_AT, 229830_AT, 205059_S_AT, |
| | 211 [symbol] 61_X_AT, 235739_AT, 213763_AT, 2050 [symbol] 7_AT, 202 [symbol] 59_X_AT, 223092_AT, 221463_AT, |
| | 209827_S_AT, 210839_S_AT, 1554992_AT, 20 [symbol] 06_AT, 20276 [symbol] _AT, 2132 [symbol] 1_AT, 204655_AT, |
| | 20 [symbol] 1 [symbol] 5_AT, 225116_AT, 201466_S_AT, 204025_AT, 225115_AT, 2204 [symbol] 4_AT, 2250 [symbol] 7_AT, 225797_AT, |
| | 2253 [symbol] _AT, 1555759_A_AT, 209 [symbol] 2_AT, 210815_S_AT, 2156 [symbol] _AT, 202539_S_AT, 2050 [symbol] _AT |
| GOTERM_{—} | 201464_X_AT, 237252_AT, 201463_S_AT, 39402_AT, 202241_AT, 2132 [symbol] 1_AT, 1405_I_AT, 204655_AT, |
| BP_ALL | 1552553_A_AT, 20 [symbol] 9_AT, 202708_S_AT, 201466_S_AT, 203 [symbol] _AT, 221661_X_AT, 222062_AT, |
| | 219 [symbol] 1 [symbol] _X_AT, 203 [symbol] 7_S_AT, 227697_AT, 205495_S_AT, 205067_AT, 1555759_A_AT, 2200 [symbol] 6_AT, |
| | 206438_S_AT |
| GOTERM_{—} | 219028_AT, 216248_S_AT, 39402_AT, 205463_S_AT, 204622_X_AT, 1405_I_AT, 211924_S_AT, |
| BP_ALL | 214866_AT, 222877_AT, 229830_AT, 205099_S_AT, 211661_X_AT, 235739_AT, 213763_AT, |
| | 205067_AT, 202859_X_AT, 223092_AT, 22 [symbol] 63_AT, 209827_S_AT, 210 [symbol] 39_S_AT, 20 [symbol] 06_AT, |
| | 20 [symbol] 655_AT, 225116_AT, 204035_AT, 225115_AT, 2204 [symbol] 4_AT, 225097_AT, 229797_AT, 2253 [symbol] _AT, |
| | 1555759_A_AT, 20 [symbol] 392_AT, 210 [symbol] 45_S_AT, 205098_AT, |
| GOTERM_{—} | 201464_X_AT, 213891_S_AT, 201465_S_AT, 227069_AT, 21902 [symbol] _AT, 21229 [symbol] _AT, 210136_AT, |
| BP_ALL | 216834_AT, 201471_S_AT, 235739_AT, 202 [symbol] 2 [symbol] _S_AT, 201067_AT, 236561_AT, 202 [symbol] 59_X_AT, |
| | 2022 [symbol] 4_S_AT, 201170_S_AT, 203729_AT, 2284 [symbol] 0_AT, 21 [symbol] 017_S_AT, 2120 [symbol] _AT, 201247_S_AT, |
| | 201565_S_AT, 20 [symbol] 753_AT, 221 [symbol] 15_AT, 2552914_A_AT, 225215_AT, 22214 [symbol] _S_AT, 205312_AT, |
| | 201005_AT, 2100 [symbol] 5_S_AT, 160020_AT, 214211_AT, 212171_X_AT, 21624 [symbol] _S_AT, 63 [symbol] 25_AT, |
| | 39402_AT, 205 [symbol] 63_S_AT, 201566_X_AT, 22 [symbol] 964_AT, 22 [symbol] 501_AT, 22339 [symbol] _AT, 22 [symbol] 30_AT, 212387_AT, |
| | 205566_AT, 21 [symbol] 502_S_AT, 239 [symbol] 1 [symbol] _X_AT, 222670_S_AT, 208476_S_AT, 200920_S_AT, 201150_S_AT, |
| | 201148_S_AT, 217757_AT, 20276 [symbol] _AT, 2132 [symbol] 1_AT, 210513_S_AT, 23 [symbol] 9_AT, 203233_AT, |
| | 2 [symbol] 451_AT, 2123 [symbol] 6_AT, 225097_AT, 219257_S_AT, 212 [symbol] 3_AT, 2175 [symbol] 1_AT, 217279_X_AT, 37152_AT, |
| | 221 [symbol] 41_S_AT, 224218_S_AT, 213763_AT, 1566901_AT, 220066_AT, 202 [symbol] _AT, 203665_AT, |
| | 204 [symbol] _S_AT, 201149_S_AT, 239519_AT, 225116_AT, 22 [symbol] 502_S_AT, 201466_S_AT, 225337_AT, |
| | 212143_S_AT, 204035_AT, 225368_AT, 202 [symbol] _S_AT, 20 [symbol] 7_S_AT, 222652_S_AT, 224859_AT, |
| | 205681_AT, 201012_AT, 204622_X_AT, 241722_X_AT, 218559_S_AT, 200748_S_AT, 203940_S_AT, |
| | 200 [symbol] 21_S_AT, 219 [symbol] 45_AT, 21990 [symbol] _AT, 210512_S_AT, 202 [symbol] 27_S_AT, [symbol] 7100_AT, 211527_X_AT, |
| | 20 [symbol] 140_AT, 220266_S_AT, 202241_AT, 2039 [symbol] 5_AT, 21025 [symbol] _AT, 2276 [symbol] 7_AT, 2263 [symbol] 2_AT, 2233 [symbol] _AT |
| GOTERM_{—} | 160020_AT, 217173_S_AT, 20114 [symbol] _S_AT, 203 [symbol] 5_AT, 39402_AT, 205463_S_AT, 201149_S_AT, |
| BP_ALL | 201147_S_AT, 20206 [symbol] _S_AT, 201044_X_AT, 229 [symbol] 30_AT, 201042_S_AT, 201313_AT, 227697_AT, |
| | 202 [symbol] 2 [symbol] _S_AT, 205067_AT, 202827_S_AT, 236561_AT, 209047_AT, 217279_X_AT, 2020 [symbol] 7_S_AT, |
| | 201150_S_AT |
| GOTERM_{—} | 2 [symbol] 1464_X_AT, 214211_AT, 201465_S_AT, 225723_AT, 37152_AT, 210136_AT, 212171_X_AT, |
| BP_ALL | 39402_AT, 1405_I_AT, 241938_AT, 34210_AT, 20 [symbol] 61_AT, 205099_S_AT, 20074 [symbol] _S_AT, 20 [symbol] 54 [symbol] _S_AT, |
| | 205067_AT, 210512_S_AT, 211527_X_AT, 200 [symbol] 7 [symbol] _AT, 201925_S_AT, 1562 [symbol] 67_AT, 217173_S_AT, |
| | 209906_AT, 216017_S_AT, 2036 [symbol] 5_AT, 2132 [symbol] 1_AT, 204655_AT, 210513_S_AT, 20206 [symbol] _S_AT, |
| | 20 [symbol] 466_S_AT, 239452_AT, 15565 [symbol] 3_A_AT, 212 [symbol] 03_AT, 1555759_A_AT, 241773_AT, 201005_AT, |
| | [symbol] |
| GOTERM_{—} | 204912_AT, 213428_S_AT, 20 [symbol] 555_S_AT, 217757_AT, 205463_S_AT, 210233_AT, 226218_AT, |
| MF_ALL | 229830_AT, 212143_S_AT, 200935_AT, 203233_AT, 206 [symbol] _S_AT, 228766_AT, 23 [symbol] 61_AT, |
| | 241929_AT, 210095_S_AT |
| GOTERM_{—} | 160020_AT, 20 [symbol] 665_AT, 201069_AT, 21624 [symbol] _S_AT, 212171_X_AT, 204527_X_AT, 205463_S_AT, |
| BP_ALL | 210513_S_AT, 229 [symbol] 30_AT, 235739_AT, 219451_AT, 2276 [symbol] 7_AT, 15565 [symbol] 3_A_AT, 202 [symbol] 2 [symbol] _S_AT, |
| | 210512_S_AT, 2022 [symbol] 4_S_AT, 202 [symbol] 27_S_AT, 236562_AT, 211527_X_AT, 217179_X_AT, 201242_S_AT, |
| | 200 [symbol] 7 [symbol] _AT, 201243_S_AT |
| GOTERM_{—} | 202540_S_AT, 20 [symbol] 55_S_AT, 205 [symbol] 52_S_AT, 2024 [symbol] 5_S_AT, 204972_AT, 2110 [symbol] 6_X_AT, 222 [symbol] 77_AT, |
| CC_ALL | 216971_ [symbol] _AT, 20 [symbol] 079_X_AT, 219412_AT, 202434_S_AT, 2064 [symbol] _S_AT, 24155 [symbol] _AT, 20 [symbol] _AT, |
| | 201925_S_AT, 20 [symbol] 5_AT, 202436_S_AT, 20 [symbol] 91_AT, 2120 [symbol] _AT, 216080_S_AT, 201500_X_AT, |
| | 2 [symbol] 8161_S_AT, 211577_AT, 22 [symbol] 502_S_AT, 236345_AT, 212 [symbol] 6_X_AT, 21 [symbol] 562_S_AT, 22 [symbol] 7 [symbol] _AT, |
| | 20550 [symbol] _AT, 2060 [symbol] _S_AT, 20 [symbol] 9 [symbol] _AT, 202 [symbol] 19_S_AT, 201324_AT, 202539_S_AT, 213503_X_AT, |
| | 224 [symbol] 67_AT, 1405_I_AT, 21 [symbol] 427_X_AT, 223501_AT, 2040 [symbol] _AT, 204 [symbol] 1_AT, [symbol] 4210_AT, 20 [symbol] _S_AT, |
| | 2 [symbol] 95_S_AT, 215 [symbol] 6_S_AT, 205212_X_AT, 1562467_AT, 1554952_AT, 20921 [symbol] _AT, 20 [symbol] 892_S_AT, |
| | 204059_S_AT, 2045 [symbol] 5_AT, 2091 [symbol] _AT, 202437_S_AT, 1554 [symbol] 00_S_AT, 2 [symbol] _AT, 204257_AT, |
| | 20131 [symbol] _AT, 212089_AT, 2394 [symbol] _AT, 20 [symbol] 81_S_AT, 155 [symbol] 5 [symbol] 3_A_AT, 202087_S_AT, 21925 [symbol] _S_AT, |
| | 1555799_A_AT, 226302_AT, 203411_S_AT, 141929_AT, 2136 [symbol] _AT |
| GOTERM_{—} | 202540_S_AT, 209555_S_AT, 202435_S_AT, 205552_S_AT, 204972_AT, 211066_X_AT, 222877_AT, |
| CC_ALL | 216971_S_AT, 209079_X_AT, 219412_AT, 202434_S_AT, 206488_S_AT, 241954_AT, 203939_AT, |
| | 203665_AT, 202436_S_AT, 212099_AT, 216080_S_AT, 208161_S_AT, 213577_AT, 236345_AT |
| | 212086_X_AT, 213562_S_AT, 228766_AT, 203508_AT, 206033_S_AT, 209993_AT, 201324_AT |
| | 207819_S_AT, 202539_S_AT, 224967_AT, 34210_AT, 204661_AT, 209994_S_AT, 215836_S_AT |
| | 205717_X_AT, 1562467_AT, 1554992_AT, 209218_AT, 209189_AT, 202437_S_AT, 1554600_S_AT |
| | 238669_AT, 204257_AT, 212089_AT, 201313_AT, 239451_AT, 204881_S_AT, 1556583_A_AT, |
| | 219257_S_AT, 203411_S_AT, 226302_AT, 241929_AT, 215688_AT |
| GOTERM_{—} | 209906_AT, 39402_AT, 205463_S_AT, 1405_I_AT, 204655_AT, 211924_S_AT, 214866_AT, 222877_AT, |
| BP_ALL | 229830_AT, 205099_S_AT, 211661_X_AT, 204035_AT, 205067_AT, 202859_X_AT, 1555759_A_AT, |
| | 209392_AT, 210845_S_AT, 221463_AT, 209827_S_AT, 210839_S_AT, 205098_AT |
| GOTERM_{—} | 209906_AT, 39402_AT, 205463_S_AT, 1405_I_AT, 204655_AT, 211924_S_AT, 214866_AT, 222877_AT, |
| BP_ALL | 229830_AT, 205099_S_AT, 211661_X_AT, 204035_AT, 205067_AT, 202859_X_AT, 1555759_A_AT, |
| | 209392_AT, 210845_S_AT, 221463_AT, 209827_S_AT, 210839_S_AT, 205098_AT |
| GOTERM_{—} | 219028_AT, 39402_AT, 216834_AT, 223398_AT, 213763_AT, 239819_X_AT, 219645_AT, 205067_AT, |
| BP_ALL | 1566901_AT, 219908_AT, 220066_AT, 202388_AT, 203140_AT, 202241_AT, 225116_AT, 212143_S_AT, |
| | 203935_AT, 225115_AT, 21025 [symbol] _AT, 227697_AT, 225097_AT, 202988_S_AT, 225368_AT, 217591_AT, |
| | 210095_S_AT |
| GOTERM_{—} | 201464_X_AT, 229221_AT, 201465_S_AT, 39402_AT, 212014_X_AT, 201473_AT, 210916_S_AT, |
| BP_ALL | 1557905_S_AT, 205067_AT, 236561_AT, 202284_S_AT, 201150_S_AT, 201148_S_AT, 204489_S_AT, |
| | 203665_AT, 204490_S_AT, 2132 [symbol] 1_AT, 201149_S_AT, 201147_S_AT, 209189_AT, 209835_X_AT, |
| | 201466_S_AT, 201313_AT, 227697_AT, 209122_AT |
| GOTERM_{—} | 201464_X_AT, 201465_S_AT, 219028_AT, 212298_AT, 219155_AT, 209555_S_AT, 216834_AT, |
| BP_ALL | 211352_S_AT, 219412_AT, 201471_S_AT, 235739_AT, 206472_S_AT, 208960_S_AT, 226389_S_AT, |
| | 205067_AT, 236561_AT, 202859_X_AT, 20 [symbol] 891_AT, 207610_S_AT, 225171_AT, 212099_AT, |
| | 220307_AT, 230266_AT, 1553982_A_AT, 207700_S_AT, 203104_AT, 225115_AT, 212977_AT, |
| | 1555832_S_AT, 228766_AT, 204834_AT, 210845_S_AT, 230925_AT, 20 [symbol] 961_S_AT, 214581_X_AT, |
| | 214297_AT, 216248_S_AT, 39402_AT, 210233_AT, 1405_I_AT, 214866_AT, 238909_AT, 223501_AT, |
| | 2020 [symbol] 6_AT, 205039_S_AT, 239818_X_AT, 221463_AT, 209906_AT, 1554992_AT, 213281_AT, |
| | 224606_AT, 211676_S_AT, 225738_AT, 216250_S_AT, 202073_AT, 203233_AT, 200872_AT, |
| | 225097_AT, 219257_S_AT, 219994_AT, 227948_AT, 238649_AT, 211924_S_AT, 201041_S_AT, |
| | 206488_S_AT, 213763_AT, 206171_AT, 225173_AT, 220066_AT, 200878_AT, 225166_AT, |
| | 204736_S_AT, 205891_AT, 203665_AT, 239519_AT, 218856_AT, 225116_AT, 201466_S_AT, |
| | 223502_S_AT, 204035_AT, 223939_AT, 225368_AT, 202988_S_AT, 241773_AT, 201012_AT, |
| | 218501_AT, 204622_X_AT, 243797_AT, 22 [symbol] 708_AT, 226218_AT, 211661_X_AT, 202686_S_AT, |
| | 20 [symbol] 93_S_AT, 225207_AT, 227265_AT, 2251 [symbol] _AT, 205419_AT, 206571_S_AT, 22 [symbol] 368_AT, |
| | 20 [symbol] 92_S_AT, 202241_AT, 204655_AT, 2091 [symbol] 9_AT, 203037_S_AT, 214054_AT, 220313_AT, |
| | 201044_X_AT, 201595_AT, 227697_AT, 1555759_A_AT, 241929_AT, 219434_AT, 215688_AT, |
| | 203320_AT, 205098_AT |
| GOTERM_{—} | 212171_X_AT, 215026_AT, 39402_AT, 1405_I_AT, 223501_AT, 204923_AT, 222062_AT, 213763_AT, |
| BP_ALL | 205067_AT, 217552_X_AT, 202859_X_AT, 210512_S_AT, 208436_S_AT, 220066_AT, 209827_S_AT, |
| | 211527_X_AT, 201925_S_AT, 209906_AT, 205891_AT, 204655_AT, 210513_S_AT, 225116_AT, |
| | 223502_S_AT, 204035_AT, 225115_AT, 225097_AT, 225368_AT, 1555759_A_AT |
| GOTERM_{—} | 160020_AT, 216248_S_AT, 39402_AT, 204622_X_AT, 205463_S_AT, 216243_S_AT, 1405_I_AT, |
| BP_ALL | 201473_AT, 204058_AT, 229630_AT, 201041_S_AT, 235739_AT, 212657_S_AT, 202828_S_AT, |
| | 205067_AT, 202284_S_AT, 202827_S_AT, 236561_AT, 209047_AT, 1562467_AT, 201150_S_AT, |
| | 217173_S_AT, 201148_S_AT, 203665_AT, 217757_AT, 204059_S_AT, 204655_AT, 201149_S_AT, |
| | 2091 [symbol] 9_AT, 201147_S_AT, 202068_S_AT, 201044_X_AT, 238669_AT, 201313_AT, 227697_AT, |
| | 1555759_A_AT, 217279_X_AT, 202067_S_AT, 207819_S_AT |
| GOTERM_{—} | 203665_AT, 205891_AT, 203940_S_AT, 39402_AT, 200921_S_AT, 212099_AT, 219257_S_AT, |
| BP_ALL | 205067_AT, 208937_S_AT, 200920_S_AT |
| GOTERM_{—} | 201464_X_AT, 201465_S_AT, 229221_AT, 216248_S_AT, 39402_AT, 204522_X_AT, 205463_S_AT, |
| BP_ALL | 221841_S_AT, 212014_X_AT, 210916_S_AT, 229830_AT, 201041_S_AT, 235739_AT, 1557905_S_AT, |
| | 202686_S_AT, 205067_AT, 202284_S_AT, 201150_S_AT, 201148_S_AT, 2044 [symbol] 9_S_AT, 203655_AT, |
| | 205891_AT, 204490_S_AT, 220266_S_AT, 217757_AT, 213281_AT, 201149_S_AT, 209189_AT, |
| | 201147_S_AT, 209835_X_AT, 201044_X_AT, 201466_S_AT, 227697_AT, 1556583_A_AT |
| GOTERM_{—} | 201464_X_AT, 213891_S_AT, 229221_AT, 201465_S_AT, 219028_AT, 212298_AT, 209555_S_AT, |
| BP_ALL | 212014_X_AT, 201473_AT, 211352_S_AT, 210916_S_AT, 201471_S_AT, 235739_AT, 208960_S_AT, |
| | 205067_AT, 217997_AT, 236561_AT, 202284_S_AT, 1569149_AT, 204489_S_AT, 201147_S_AT, |
| | 201565_S_AT, 203753_AT, 207700_S_AT, 1552914_A_AT, 225115_AT, 1555832_S_AT, 228766_AT, |
| | 222146_S_AT, 205312_AT, 243536_AT, 208961_S_AT, 210095_S_AT, 212171_X_AT, 216248_S_AT, |
| | 39402_AT, 205463_S_AT, 1405_I_AT, 235457_AT, 201566_X_AT, 223501_AT, 2296830_AT, 2123 [symbol] 7_AT, |
| | 2020 [symbol] 6_AT, 1557905_S_AT, 222670_S_AT, 200920_S_AT, 201150_S_AT, 203751_X_AT, 217996_AT, |
| | 20114 [symbol] _S_AT, 1554992_AT, 213281_AT, 224606_AT, 210513_S_AT, 1565754_X_AT, 225842_AT, |
| | 235295_AT, 1565752_AT, 203233_AT, 204105_S_AT, 212386_AT, 225097_AT, 203752_S_AT, |
| | 219257_S_AT, 225557_AT, 37152_AT, 227948_AT, 221841_S_AT, 242794_AT, 21294 [symbol] _AT, 204923_AT, |
| | 201041_S_AT, 206488_S_AT, 213763_AT, 1554453_AT, 1566901_AT, 220066_AT, 200878_AT, |
| | 205891_AT, 203665_AT, 204490_S_AT, 201149_S_AT, 239519_AT, 209835_X_AT, 225116_AT, |
| | 204715_AT, 206157_AT, 201466_S_AT, 223502_S_AT, 212143_S_AT, 204035_AT, 215602_AT, |
| | 212124_AT, 225368_AT, 241773_AT, 224859_AT, 218501_AT, 243797_AT, 204622_X_AT, 227240_AT, |
| | 226218_AT, 218559_S_AT, 227107_AT, 204401_AT, 200921_S_AT, 203548_S_AT, 210512_S_AT, |
| | 211527_X_AT, 244414_AT, 203140_AT, 242405_AT, 220266_S_AT, 204655_AT, 1552553_A_AT, |
| | 209189_AT, 201044_X_AT, 203935_AT, 227697_AT, 1555759_A_AT, 241929_AT, 21563 [symbol] _AT, |
| | 223394_AT |
| GOTERM_{—} | 202540_S_AT, 209555_S_AT, 202435_S_AT, 205552_S_AT, 204972_AT, 211066_X_AT, 222877_AT, |
| CC_ALL | 209079_X_AT, 219412_AT, 202434_S_AT, 206488_S_AT, 241954_AT, 203939_AT, 203665_AT, |
| | 202436_S_AT, 212099_AT, 216080_S_AT, 208161_S_AT, 213577_AT, 236345_AT, 213562_S_AT, |
| | 228766_AT, 203508_AT, 206033_S_AT, 201324_AT, 207819_S_AT, 209993_AT, 202539_S_AT, |
| | 224967_AT, 34210_AT, 204661_AT, 209994_S_AT, 215836_S_AT, 205717_X_AT, 1562467_AT, |
| | 1554992_AT, 209218_AT, 209189_AT, 202437_S_AT, 238669_AT, 204257_AT, 201313_AT, 239451_AT, |
| | 204881_S_AT, 1556583_A_AT, 219257_S_AT, 226302_AT, 241929_AT, 215688_AT |
| GOTERM_{—} | 202540_S_AT, 223723_AT, 227069_AT, 212298_AT, 212014_X_AT, 221731_X_AT, 203923_S_AT, 219412_AT, |
| CC_ALL | 207674_AT, 2186 [symbol] 1_AT, 209803_S_AT, 226545_AT, 203729_AT, 217173_S_AT, 2044 [symbol] 9_S_AT, 205505_AT, |
| | 204620_S_AT, 220307_AT, 223303_AT, 211307_S_AT, 216080_S_AT, 232649_AT, 203922_S_AT, 21 [symbol] 6 [symbol] 6_S_AT, |
| | 221 [symbol] 15_AT, 1552 [symbol] 14_A_AT, 203104_AT, 2135 [symbol] 2_S_AT, 202756_S_AT, 22 [symbol] 766_AT, 224674_AT, 2145 [symbol] 1_X_AT, |
| | 224967_AT, 63825_AT, 211719_X_AT, 209949_AT, 223501_AT, 205099_S_AT, 1557 [symbol] 05_S_AT, 215836_S_AT, |
| | 238542_AT, 209670_AT, 209047_AT, 240076_AT, 217078_S_AT, 221 [symbol] 40_AT, 201626_AT, 219926_AT, 211676_S_AT, |
| | 238669_AT, 204257_AT, 203233_AT, 204105_S_AT, 224989_AT, 239761_AT, 201263_S_AT, 204912_AT, 23861 [symbol] _AT, |
| | 222877_AT, 209079_X_AT, 212062_AT, 22 [symbol] 0 [symbol] 3_AT, 202434_S_AT, 204222_S_AT, 2064 [symbol] _S_AT, 22 [symbol] 640_AT, |
| | 205542_AT, 203665_AT, 204736_S_AT, 205 [symbol] 1_AT, 204359_AT, 202748_AT, 239519_AT, 20 [symbol] 35_X_AT, |
| | 20 [symbol] 262_S_AT, 218 [symbol] 56_AT, 204715_AT, 223502_S_AT, 236345_AT, 223939_AT, 203 [symbol] 7_S_AT, 204774_AT, |
| | 20253 [symbol] _S_AT, 22621 [symbol] _AT, 204661_AT, 225 [symbol] 09_AT, 204401_AT, 202 [symbol] _S_AT, 222062_AT, 2251 [symbol] _AT, |
| | 217552_X_AT, 202071_AT, 2249 [symbol] 0_AT, 225325_AT, 220313_AT, 20571 [symbol] _AT, 203 [symbol] 35_AT, 1556583_A_AT, |
| | 201995_AT, 229797_AT, 226302_AT, 210173_AT, 2050 [symbol] 8_AT, 210495_X_AT, 229221_AT, 210136_AT, 209555_S_AT, |
| | 202435_S_AT, 211066_X_AT, 210916_S_AT, 2244 [symbol] 0_S_AT, 21 [symbol] 225_AT, 20282 [symbol] _S_AT, 242 [symbol] 71_AT, 214255_AT, |
| | 236561_AT, 230360_AT, 201925_S_AT, 22 [symbol] 0_AT, 207610_S_AT, 212099_AT, 213577_AT, 229 [symbol] 37_X_AT, |
| | 214 [symbol] 30_AT, 227052_AT, 212977_AT, 20350 [symbol] _AT, 2 [symbol] 0925_AT, 210 [symbol] 5_S_AT, 2010 [symbol] 5_AT, 206033_S_AT, |
| | 201242_S_AT, 233220_AT, 207819_S_AT, 242907_AT, 209122_AT, 160020_AT, 237252_AT, 214297_AT, 210233_AT, |
| | 22 [symbol] 910_AT, 214 [symbol] 66_AT, 205534_AT, 34210_AT, 205566_AT, 209994_S_AT, 1552690_A_AT, 22 [symbol] 754_AT, 217678_AT, |
| | 205717_X_AT, 2385 [symbol] 1_AT, 209906_AT, 209218_AT, 215646_S_AT, 204619_S_AT, 205520_AT, 213030_S_AT, |
| | 20 [symbol] 121_S_AT, 235295_AT, 20 [symbol] 1_S_AT, 239451_AT, 219994_AT, 217279_X_AT, 201125_S_AT, 202627_S_AT, |
| | 231924_S_AT, 203217_S_AT, 206171_AT, 241554_AT, 203 [symbol] 3 [symbol] _AT, 2 [symbol] 54_AT, 202436_S_AT, 204490_S_AT, |
| | 219386_S_AT, 20 [symbol] 921_AT, 225337_AT, 2204 [symbol] 4_AT, 209145_AT, 241773_AT, 22 [symbol] 762_AT, 20 [symbol] 392_AT, 224859_AT, |
| | 201625_S_AT, 201324_AT, 209993_AT, 201012_AT, 212464_S_AT, 216442_X_AT, 227180_AT, 241722_X_AT, |
| | 22 [symbol] 708_AT, 214724_AT, 227107_AT, 211661_X_AT, 226 [symbol] 31_AT, 20354 [symbol] _S_AT, 2193 [symbol] 5_AT, 202827_S_AT, |
| | 2230 [symbol] 2_AT, [symbol] 7200_AT, 205419_AT, 210 [symbol] 39_S_AT, 235299_AT, 219332_AT, 202437_S_AT, 20206 [symbol] _S_AT, 20 [symbol] _AT, |
| | 223798_AT, 22 [symbol] 696_AT, 241929_AT, 202067_S_AT, 219434_AT, 211571_S_AT |
| GOTERM_{—} | 160020_AT, 203665_AT, 201069_AT, 216248_S_AT, 212171_X_AT, 204622_X_AT, 205463_S_AT, |
| BP_ALL | 220513_S_AT, 229 [symbol] 30_AT, 235739_AT, 23 [symbol] 451_AT, 15565 [symbol] 3_A_AT, 227697_AT, 202 [symbol] 2 [symbol] _S_AT, |
| | 202827_S_AT, 210512_S_AT, 236561_AT, 217279_X_AT, 211527_X_AT, 201242_S_AT, 2 [symbol] 78_AT, |
| | 201243_S_AT |
| GOTERM_{—} | 20 [symbol] 06_AT, 20 [symbol] 5_AT, 20 [symbol] 140_AT, 205 [symbol] 1_AT, 39402_AT, 217757_AT, 22621 [symbol] _AT, 223501_AT, |
| BP_ALL | 223502_S_AT, 1552 [symbol] 14_A_AT, 204923_AT, 203233_AT, 222062_AT, 205067_AT, 217552_X_AT, |
| | 220066_AT, 224 [symbol] 59_AT, 201925_S_AT |
| GOTERM_{—} | 219028_AT, 39402_AT, 216 [symbol] 34_AT, 22 [symbol] 8_AT, 213 [symbol] 63_AT, 239 [symbol] 1 [symbol] _X_AT, 219 [symbol] 45_AT, 2050 [symbol] 7_AT, |
| BP_ALL | 15 [symbol] 01_AT, 21 [symbol] 0 [symbol] _AT, 220066_AT, 202 [symbol] _AT, 20 [symbol] 140_AT, 202241_AT, 22511 [symbol] _AT, 21214 [symbol] _S_AT, |
| | 2039 [symbol] 5_AT, 225115_AT, 21025 [symbol] _AT, 227697_AT, 2250 [symbol] 7_AT, 2029 [symbol] _S_AT, 22536 [symbol] _AT, 2175 [symbol] 1_AT, |
| | 2100 [symbol] 5_S_AT |
| GOTERM_{—} | 203140_AT, 222062_AT, 227 [symbol] 97_AT, 39402_AT, 217757_AT, 20350 [symbol] _AT, 205067_AT, 22621 [symbol] _AT, |
| BP_ALL | 224 [symbol] _AT, 20 [symbol] 9_AT, 233220_AT, 1552914_A_AT |
| GOTERM_{—} | 20114 [symbol] _S_AT, 204620_S_AT, 20114 [symbol] _S_AT, 221924_S_AT, 201147_S_AT, 215646_S_AT, 204619_S_AT, |
| BP_ALL | 221731_X_AT, 214 [symbol] 6_AT, 20 [symbol] 6_S_AT, 227 [symbol] 7_AT, 2022 [symbol] 4_S_AT, 236561_AT, 210 [symbol] 45_S_AT, |
| | 211571_S_AT, 201150_S_AT, 20 [symbol] 5_S_AT |
| GOTERM_{—} | 160020_AT, 39402_AT, 205 [symbol] 3_S_AT, 2162 [symbol] 3_S_AT, 1405_I_AT, 20147 [symbol] _AT, 204 [symbol] 5 [symbol] _AT, 22 [symbol] 30_AT, |
| BP_ALL | 201041_S_AT, 212657_S_AT, 202 [symbol] 2 [symbol] _S_AT, 205067_AT, 2022 [symbol] 4_S_AT, 202 [symbol] 27_S_AT, 236561_AT, |
| | 209047_AT, 201150_S_AT, 217173_S_AT, 2011 [symbol] _S_AT, 203665_AT, 217757_AT, 204059_S_AT, |
| | 204 [symbol] 55_AT, 2011 [symbol] _S_AT, 2091 [symbol] _AT, 201147_S_AT, 20206 [symbol] _S_AT, 2010 [symbol] 4_X_AT, 23 [symbol] _AT, |
| | 201313_AT, 227 [symbol] 7_AT, 155575 [symbol] _A_AT, 217279_X_AT, 202067_S_AT, 207 [symbol] 19_S_AT |
| GOTERM_{—} | 214211_AT, 201464_X_AT, 223723_AT, 2014 [symbol] 5_S_AT, 37152_AT, 2101 [symbol] 6_AT, [symbol] 402_AT, 1 [symbol] 5_I_AT, |
| BP_ALL | 242938_AT, 20 [symbol] 661_AT, 34220_AT, 2050 [symbol] 9_S_AT, 2007 [symbol] _S_AT, 205067_AT, 201925_S_AT, 1562467_AT, |
| | 20 [symbol] 06_AT, 216017_S_AT, 203 [symbol] 5_AT, 213281_AT, 204655_AT, 201 [symbol] _S_AT, 239451_AT, |
| | 155 [symbol] 3_A_AT, 212 [symbol] _AT, 155575 [symbol] _A_AT, 241773_AT, 201005_AT, 2050 [symbol] _AT |
| GOTERM_{—} | 210 [symbol] 5_X_AT, 22 [symbol] 221_AT, 37152_AT, 21229 [symbol] _AT, 2 [symbol] 555_S_AT, 212014_S_AT, 221731_X_AT, |
| BP_ALL | 2110 [symbol] _X_AT, 222877_AT, 210 [symbol] _S_AT, 209079_X_AT, 2064 [symbol] _S_AT, 228640_AT, 2044 [symbol] _S_AT, |
| | 21342 [symbol] _S_AT, 20 [symbol] 35 [symbol] _AT, 2044 [symbol] 0_S_AT, 204620_S_AT, 2120 [symbol] _AT, 22 [symbol] _AT, 23 [symbol] 519_AT, |
| | 20 [symbol] 15_X_AT, 207571_X_AT, 22 [symbol] 7 [symbol] _AT, 22 [symbol] 73_AT, 206013_S_AT, 201005_AT, 212464_S_AT, |
| | 216442_X_AT, 1405_I_AT, 21171 [symbol] _X_AT, 205534_AT, 2050 [symbol] 9_S_AT, 15579 [symbol] 5_S_AT, 215 [symbol] 36_S_AT, |
| | 205717_X_AT, 2251 [symbol] _AT, 21707 [symbol] _S_AT, 204655_AT, 215646_S_AT, 20 [symbol] 619_S_AT, 2107 [symbol] 5_S_AT, |
| | 20 [symbol] 037_S_AT, 216250_S_AT, 20 [symbol] 71 [symbol] _AT, 2 [symbol] 41 [symbol] 5_S_AT, 155575 [symbol] _A_AT, 241929_A_AT, 201125_S_AT, |
| | 211571_S_AT, 2050 [symbol] _AT |
| GOTERM_{—} | 2104 [symbol] 5_X_AT, 229211_AT, 37152_AT, 2122 [symbol] _AT, 20 [symbol] 555_S_AT, 212014_X_AT, 221731_X_AT, |
| BP_ALL | 212066_X_AT, 222 [symbol] 77_AT, 210916_S_AT, 2 [symbol] 9079_X_AT, 206 [symbol] _S_AT, 22 [symbol] 640_AT, 2044 [symbol] _S_AT; |
| | 21342 [symbol] _S_AT, 20435 [symbol] _AT, 20 [symbol] 490_S_AT, 204620_S_AT, 2120 [symbol] _AT, 223 [symbol] 03_AT, 239519_AT, |
| | 20 [symbol] 35_X_AT, 207571_X_AT, 228766_AT, 22887 [symbol] _AT, 206013_S_AT, 201005_AT, 212464_S_AT, |
| | 216442_X_AT, 1405_I_AT, 211719_X_AT, 205534_AT, 2050 [symbol] 9_S_AT, 1557 [symbol] 05_S_AT, 215 [symbol] 36_S_AT, |
| | 205717_X_AT, 22 [symbol] 1 [symbol] _AT, 217070_S_AT, 2 [symbol] 4 [symbol] 55_AT, 2156 [symbol] _S_AT, 204 [symbol] 19_S_AT, 210785_S_AT, |
| | 203037_S_AT, 216250_S_AT, 20571 [symbol] _AT, 204105_S_AT, 155575 [symbol] _A_AT, 24192 [symbol] _AT, 201125_S_AT, |
| | 211571_S_AT, 2050 [symbol] _AT |
| GOTERM_{—} | 214211_AT, 201 [symbol] 64_X_AT, 22 [symbol] 723_AT, 2014 [symbol] _S_AT, 37152_AT, 210 [symbol] 6_AT, 39402_AT, 1405_I_AT, |
| BP_ALL | 241 [symbol] _AT, 204661_AT, 34210_AT, 2050 [symbol] _S_AT, 2007 [symbol] _S_AT, 205067_AT, 201 [symbol] 25_S_AT, 1562 [symbol] 67_AT, |
| | 20 [symbol] 906_AT, 21 [symbol] 017_S_AT, 2132 [symbol] 1_AT, 2 [symbol] 655_AT, 20146 [symbol] _S_AT, 23 [symbol] 452_AT, 155 [symbol] _A_AT, |
| | 212 [symbol] _AT, 155575 [symbol] _A_AT, 2417 [symbol] _AT, 20100 [symbol] _AT, 2 [symbol] 0 [symbol] _AT |
| GOTERM_{—} | 214231_AT, 201464_X_AT, 223723_AT, 201465_S_AT, 37152_AT, 210136_AT, 39402_AT, 1405_I_AT |
| BP_ALL | 241938_AT, 204661_AT, 34210_AT, 205099_S_AT, 200748_S_AT, 205067_AT, 201925_S_AT, 1562467_AT |
| | 209906_AT, 216017_S_AT, 213281_AT, 204655_AT, 201466_S_AT, 239451_AT, 1556583_A_AT |
| | 212803_AT, 1555759_A_AT, 241773_AT, 201005_AT, 205098_AT |
| GOTERM_{—} | 209906_AT, 205891_AT, 203140_AT, 212171_X_AT, 39402_AT, 210513_S_AT, 226218_AT, 223501_AT |
| BP_ALL | 223502_S_AT, 1552914_A_AT, 204923_AT, 203233_AT, 222062_AT, 205067_AT, 210512_S_AT |
| | 202284_S_AT, 217552_X_AT, 220066_AT, 211527_X_AT, 224859_AT, 201925_S_AT |
| GOTERM_{—} | 203665_AT, 205891_AT, 203140_AT, 222062_AT, 39402_AT, 205067_AT, 220066_AT, 226218_AT |
| BP_ALL | 204923_AT |
| GOTERM_{—} | 237252_AT, 201464_X_AT, 201465_S_AT, 39402_AT, 202241_AT, 213281_AT, 1405_I_AT, 204655_AT |
| BP_ALL | 209189_AT, 201466_S_AT, 203888_AT, 211661_X_AT, 239818_X_AT, 203887_S_AT, 227697_AT |
| | 205067_AT, 1555759_A_AT, 220066_AT |
| KEGG_{—} | 229221_AT, 204489_S_AT, 204490_S_AT, 209555_S_AT, 39402_AT, 212014_X_AT, 209835_X_AT |
| PATHWAY | 226218_AT, 210916_S_AT, 203104_AT, 1557905_S_AT, 203233_AT, 206488_S_AT, 228766_AT, 205067_AT |
| | 217552_X_AT, 241929_AT, 201005_AT, 201925_S_AT |
| GOTERM_{—} | 203140_AT, 203233_AT, 222062_AT, 39402_AT, 205067_AT, 220066_AT, 226218_AT, 223501_AT |
| BP_ALL | 223502_S_AT, 204923_AT |
| GOTERM_{—} | 201464_X_AT, 237252_AT, 201465_S_AT, 37152_AT, 219028_AT, 39402_AT, 1405_I_AT, 201473_AT |
| BP_ALL | 202708_S_AT, 202086_AT, 211661_X_AT, 213763_AT, 203882_AT, 222062_AT, 239818_X_AT, 205067_AT, |
| | 236561_AT, 208436_S_AT, 220066_AT, 209827_S_AT, 204475_AT, 217173_S_AT, 214453_S_AT, |
| | 213281_AT, 202241_AT, 204655_AT, 1552553_A_AT, 209189_AT, 202068_S_AT, 211676_S_AT, |
| | 225116_AT, 229937_X_AT, 201466_S_AT, 206157_AT, 203888_AT, 225115_AT, 212977_AT, 203887_S_AT, |
| | 227697_AT, 225097_AT, 205495_S_AT, 225368_AT, 1555759_A_AT, 202067_S_AT, 208438_S_AT, |
| | 213293_S_AT |
| GOTERM_{—} | 201464_X_AT, 202540_S_AT, 201465_S_AT, 37152_AT, 210136_AT, 202435_S_AT, 241938_AT, 212948_AT, |
| BP_ALL | 228083_AT, 202434_S_AT, 205067_AT, 200878_AT, 217173_S_AT, 216017_S_AT, 205891_AT, 203665_AT, |
| | 202436_S_AT, 201149_S_AT, 201147_S_AT, 201466_S_AT, 220484_AT, 201005_AT, 233220_AT, |
| | 202539_S_AT, 212171_X_AT, 39402_AT, 204401_AT, 1552690_A_AT, 206835_AT, 204684_AT, 235944_AT, |
| | 210512_S_AT, 209047_AT, 211527_X_AT, 201150_S_AT, 1562467_AT, 201148_S_AT, 1554992_AT, |
| | 209906_AT, 213281_AT, 202437_S_AT, 209189_AT, 210513_S_AT, 2020268_S_AT, 212372_AT, 238669_AT, |
| | 1556583_A_AT, 229797_AT, 212803_AT, 202067_S_AT, 215688_AT |
| GOTERM_{—} | 213 [symbol] 1_S_AT, 201464_X_AT, 2025 [symbol] 0_S_AT, 2270 [symbol] _AT, 2014 [symbol] 5_S_AT, 219028_AT, 21229 [symbol] _AT, 212014_X_AT, |
| BP_ALL | 216 [symbol] 34_AT, 219412_AT, 20 [symbol] 60_S_AT, 217 [symbol] 93_AT, 205067_AT, 202 [symbol] 59_X_AT, 202170_S_AT, 20 [symbol] 729_AT, 2044 [symbol] _S_AT, |
| | 116017_S_AT, 225171_AT, 220 [symbol] 07_AT, 201565_S_AT, 201147_S_AT, 230266_AT, 204 [symbol] 3_AT, 221 [symbol] 15_AT, 207700_S_AT, |
| | 1552914_A_AT, 20 [symbol] 104_AT, 1555 [symbol] 2_S_AT, 204 [symbol] 34_AT, 2221 [symbol] _S_AT, 22 [symbol] 76 [symbol] _AT, 210095_S_AT, 214211_AT, |
| | 2145 [symbol] 1_X_AT, 21624 [symbol] _S_AT, 63 [symbol] 25_AT, 203 [symbol] 63_S_AT, 1405_I_AT, 201566_X_AT, 22 [symbol] 964_AT, 211719_X_AT, 223501_AT, |
| | 2020 [symbol] _AT, 212 [symbol] 7_AT, 220 [symbol] 0_AT, 2000 [symbol] 9_S_AT, 1557 [symbol] 05_S_AT, 21 [symbol] 502_S_AT, 222670_S_AT, 200920_S_AT, |
| | 2191 [symbol] 3_S_AT, 20 [symbol] _S_AT, 217 [symbol] 6_AT, 203751_X_AT, 201150_S_AT, 2011 [symbol] _S_AT, 155 [symbol] 2_AT, 217757_AT, |
| | 22 [symbol] 3_AT, 1565754_X_AT, 20 [symbol] 606_AT, 213676_S_AT, 225 [symbol] 2_AT, 22573 [symbol] _AT, 216250_S_AT, 202073_AT, 23 [symbol] 6 [symbol] _AT, |
| | 1565752_AT, 203233_AT, 204105_S_AT, 3702 [symbol] _AT, 20 [symbol] 752_S_AT, 225557_AT, 2175 [symbol] 1_AT, 37152_AT, 227 [symbol] _AT, |
| | 24279 [symbol] _AT, 2419 [symbol] _AT, 201041_S_AT, 204 [symbol] 23_AT, 20 [symbol] 8_S_AT, 2119 [symbol] 2_S_AT, 225173_AT, 220066_AT, 20067 [symbol] _AT, |
| | 2251 [symbol] _AT, 202 [symbol] _AT, 203 [symbol] _AT, 20473 [symbol] _S_AT, 205 [symbol] 1_AT, 21 [symbol] 529_AT, 209 [symbol] 5_X_AT, 21 [symbol] 5 [symbol] _AT, 225116_AT, |
| | 2121 [symbol] 0_AT, 204715_AT, 223502_S_AT, 201466_S_AT, 212143_S_AT, 215 [symbol] 02_AT, 223 [symbol] _AT, 202 [symbol] _S_AT, |
| | 20 [symbol] 17_S_AT, 222651_S_AT, 202 [symbol] _S_AT, 21 [symbol] 502_AT, 2437 [symbol] 7_AT, 227340_AT, 22 [symbol] 213_AT, 20074 [symbol] _S_AT, 204401_AT, |
| | 202 [symbol] _S_AT, 222062_AT, 21 [symbol] 45_AT, 2272 [symbol] 5_AT, 21 [symbol] 0 [symbol] _AT, 210512_S_AT, 2251 [symbol] _AT, 211527_X_AT, 202072_AT, |
| | 244414_AT, 203140_AT, 20 [symbol] 2_S_AT, 2202 [symbol] _S_AT, 242 [symbol] 0 [symbol] _AT, 202241_AT, 201044_X_AT, 220 [symbol] 13_AT, 20 [symbol] _AT, |
| | 220 [symbol] _AT, 201 [symbol] _AT, 226302_AT, 215 [symbol] _AT, 2050 [symbol] _AT, 210 [symbol] 5_X_AT, 229221_AT, 2101 [symbol] 6_AT, 20 [symbol] _S_AT, |
| | 219155_AT, 221011_S_AT, 201473_AT, 20247 [symbol] _AT, 210 [symbol] 36_S_AT, 211352_S_AT, 1555 [symbol] 27_AT, 201471_S_AT, 23573 [symbol] _AT, |
| | 206472_S_AT, 203 [symbol] 7_AT, 22 [symbol] 9_S_AT, 2244 [symbol] 0_S_AT, 202 [symbol] 2 [symbol] _S_AT, 214255_AT, 2022 [symbol] 4_S_AT, 236561_AT, |
| | 209 [symbol] 27_S_AT, 156 [symbol] 149_AT, 22 [symbol] 490_AT, 207 [symbol] 10_S_AT, 20 [symbol] 1_AT, 2120 [symbol] _AT, 20 [symbol] 753_AT, 155 [symbol] _A_AT, 225125_AT, |
| | 212 [symbol] 77_AT, 20550 [symbol] _AT, 205312_AT, 230 [symbol] 25_AT, 210 [symbol] 45_S_AT, 24 [symbol] 556_AT, 201005_AT, 20 [symbol] 61_S_AT, 21529 [symbol] _S_AT, |
| | 160020_AT, 212171_X_AT, 214297_AT, 39402_AT, 2102 [symbol] _AT, 235457_AT, 214 [symbol] 66_AT, 23 [symbol] 0 [symbol] _AT, 202191_S_AT, |
| | 2233 [symbol] _AT, 20 [symbol] 6_AT, 23 [symbol] 1 [symbol] _X_AT, 221 [symbol] 63_AT, 20 [symbol] 436_S_AT, 2027 [symbol] 06_AT, 20 [symbol] _AT, 224606_AT, 210511_S_AT, |
| | 2352 [symbol] 5_AT, 23 [symbol] 451_AT, 200 [symbol] 72_AT, 2123 [symbol] 6_AT, 2250 [symbol] 7_AT, 219257_S_AT, 21 [symbol] 4_AT, 212 [symbol] 0 [symbol] _AT, 217279_X_AT, |
| | 221841_S_AT, 23 [symbol] 49_AT, 211924_S_AT, 22421 [symbol] _S_AT, 2129 [symbol] _AT, 213763_AT, 206171_AT, 1564433_AT, 156 [symbol] 01_AT, |
| | 1560226_AT, 202014_AT, 2044 [symbol] 0_S_AT, 2049 [symbol] _S_AT, 20114 [symbol] _S_AT, 23 [symbol] 570_AT, 20 [symbol] 157_AT, 225 [symbol] 7_AT, 204235_AT, |
| | 212124_AT, 22536 [symbol] _AT, 241773_AT, 20 [symbol] 292_AT, 224 [symbol] 59_AT, 201012_AT, 205681_AT, 212464_S_AT, 216442_X_AT, |
| | 204622_X_AT, 241722_X_AT, 22 [symbol] 70 [symbol] _AT, 21 [symbol] 559_S_AT, 227107_AT, 211 [symbol] 1_X_AT, 20 [symbol] 40_S_AT, 20 [symbol] 3_S_AT, |
| | 225207_AT, 200 [symbol] 21_S_AT, 203548_S_AT, 202 [symbol] 27_S_AT, 20541 [symbol] _AT, [symbol] 7100_AT, 1562467_AT, 210 [symbol] _S_AT, |
| | 206571_S_AT, 22 [symbol] 36 [symbol] _AT, 224701_AT, 220 [symbol] 35_S_AT, 204 [symbol] _AT, 1552553_A_AT, 20 [symbol] 1 [symbol] _AT, 214054_AT, 203037_S_AT, |
| | 212372_AT, 2276 [symbol] 7_AT, 155575 [symbol] _A_AT, 241 [symbol] 29_AT, 21943 [symbol] _AT, 20 [symbol] 20_AT, 22 [symbol] _AT |
| GOTERM_{—} | 203140_AT, 203233_AT, 222062_AT, 39402_AT, 205067_AT, 220066_AT, 226218_AT, 223501_AT, |
| BP_ALL | 223502_S_AT, 204923_AT |
| GOTERM_{—} | 223723_AT, 212099_AT, 228708_AT, 211924_S_AT, 214866_AT, 34210_AT, 204661_AT, 202756_S_AT, |
| CC_ALL | 203548_S_AT, 238542_AT, 226545_AT, 210 [symbol] 45_S_AT, 203939_AT, 201925_S_AT |
| GOTERM_{—} | 201464_X_AT, 214211_AT, 201465_S_AT, 223723_AT, 37152_AT, 212171_X_AT, 210136_AT, |
| BP_ALL | 39402_AT, 241722_X_AT, 1405_I_AT, 201566_X_AT, 22621 [symbol] _AT, 223501_AT, 241938_AT, 204661_AT, |
| | 34210_AT, 20074 [symbol] _S_AT, 205099_S_AT, 204401_AT, 2035 [symbol] 8_S_AT, 205067_AT, 210512_S_AT, |
| | 211527_X_AT, 20087 [symbol] _AT, 201925_S_AT, 1562467_AT, 217173_S_AT, 209906_AT, 216017_S_AT, |
| | 203665_AT, 203140_AT, 2132 [symbol] 1_AT, 204655_AT, 210513_S_AT, 201565_S_AT, 202068_S_AT, |
| | 223502_S_AT, 201466_S_AT, 239451_AT, 15565 [symbol] 3_A_AT, 212803_AT, 1555759_A_AT, 241773_AT, |
| | 201005_AT, 202067_S_AT, 20509 [symbol] _AT |
| GOTERM_{—} | 206488_S_AT, 37152_AT, 209555_S_AT, 22 [symbol] 754_AT, 22 [symbol] 766_AT, 226302_AT, 211030_S_AT |
| BP_ALL | 205920_AT, 241929_AT, 208161_S_AT, 201012_AT, 209122_AT |
| GOTERM_{—} | 212171_X_AT, 63825_AT, 205463_S_AT, 229 [symbol] 30_AT, 205566_AT, 203940_S_AT, 239 [symbol] 18_X_AT, |
| BP_ALL | 202859_X_AT, 210512_S_AT, 87100_AT, 209827_S_AT, 211527_X_AT, 210 [symbol] 39_S_AT, 228490_AT, |
| | 203665_AT, 202241_AT, 210513_S_AT, 212190_AT, 221815_AT, 225337_AT, 212143_S_AT, |
| | 204035_AT, 219257_S_AT, 209392_AT, 210095_S_AT |
| GOTERM_{—} | 213 [symbol] 91_S_AT, 201464_X_AT, 227069_AT, 201465_S_AT, 37152_AT, 21902 [symbol] _AT, 221 [symbol] 41_S_AT, |
| BP_ALL | 201473_AT, 242794_AT, 21294 [symbol] _AT, 201471_S_AT, 235739_AT, 20 [symbol] 960_S_AT, 213763_AT, |
| | 1566901_AT, 200 [symbol] 7 [symbol] _AT, 203665_AT, 2049 [symbol] _S_AT, 201565_S_AT, 203753_AT, 204653_AT, |
| | 225116_AT, 201466_S_AT, 225115_AT, 1555 [symbol] 32_S_AT; 222146_S_AT, 212124_AT, 22536 [symbol] _AT, |
| | 205312_AT, 20 [symbol] 37_S_AT, 20 [symbol] 61_S_AT, 21624 [symbol] _S_AT, 212171_X_AT, 204622_X_AT, 235457_AT, |
| | 201566_X_AT, 22 [symbol] 64_AT, 212387_AT, 218559_S_AT, 222670_S_AT, 210512_S_AT, 20 [symbol] 436_S_AT, |
| | 211527_X_AT, 203751_X_AT, 244414_AT, 203140_AT, 242405_AT, 220266_S_AT, 20276 [symbol] _AT, |
| | 2132 [symbol] 1_AT, 224606_AT, 209189_AT, 210513_S_AT, 212386_AT, 225097_AT, 203752_S_AT, |
| | 225557_AT, 217591_AT |
| GOTERM_{—} | 213 [symbol] 91_S_AT, 201464_X_AT, 201465_S_AT, 219028_AT, 221 [symbol] 41_S_AT, 201473_AT, 242794_AT, |
| BP_ALL | 211352_S_AT, 212948_AT, 201471_S_AT, 23573 [symbol] _AT, 20 [symbol] 960_S_AT, 213763_AT, 205067_AT, |
| | 236561_AT, 220066_AT, 200 [symbol] 78_AT, 205 [symbol] 92_AT, 20 [symbol] 65_AT, 203753_AT, 225116_AT, 206157_AT, |
| | 201466_S_AT, 1552914_A_AT, 207700_S_AT, 225115_AT, 1555 [symbol] 32_S_AT, 222146_S_AT, 212124_AT, |
| | 22536 [symbol] _AT, 205312_AT, 20 [symbol] 961_S_AT, 224 [symbol] 59_AT, 21624 [symbol] _S_AT, 212171_X_AT, 39402_AT |
| | 204622_X_AT, 205463_S_AT, 235457_AT, 2123 [symbol] 7_AT, 22 [symbol] 30_AT, 218559_S_AT, 222670_S_AT, |
| | 210512_S_AT, 211527_X_AT, 244414_AT, 242405_AT, 220266_S_AT, 213281_AT, 224606_AT, |
| | 2091 [symbol] 9_AT, 210513_S_AT, 203 [symbol] 35_AT, 2123 [symbol] 6_AT, 225097_AT, 225557_AT, 223394_AT |
| GOTERM_{—} | 237252_AT, 213503_X_AT, 209555_S_AT, 208816_X_AT, 210427_X_AT, 211924_S_AT, 201590_X_AT, |
| BP_ALL | 214 [symbol] 66_AT, 203074_AT, 20 [symbol] _AT, 204401_AT, 2054 [symbol] _S_AT, 206 [symbol] 35_AT, 203 [symbol] 7_S_AT, |
| | 22 [symbol] 766_AT, 235944_AT, 241929_AT, 210845_S_AT, 201005_AT |
| GOTERM_{—} | 217173_S_AT, 37152_AT, 20 [symbol] 555_S_AT, 39402_AT, 219155_AT, 238649_AT, 20206 [symbol] _S_AT, |
| BP_ALL | 212062_AT, 204401_AT, 226390_AT, 206 [symbol] _S_AT, 22 [symbol] 766_AT, 214255_AT, 205067_AT, 226302_AT, |
| | 241929_AT, 202067_S_AT, 201012_AT, 209122_AT |
| GOTERM_{—} | 203665_AT, 203140_AT, 205 [symbol] 91_AT, 39402_AT, 205463_S_AT, 226218_AT, 223501_AT, 229 [symbol] 30_AT, |
| BP_ALL | 223502_S_AT, 204923_AT, 1552914_A_AT, 203233_AT, 222062_AT, 205067_AT, 2022 [symbol] _S_AT, |
| | 22 [symbol] 59_AT |
| GOTERM_{—} | 237252_AT, 201464_X_AT, 201465_S_AT, 39402_AT, 202241_AT, 2132 [symbol] 3_AT, 1405_I_AT, 204655_AT, |
| BP_ALL | 20 [symbol] 189_AT, 201466_S_AT, 203 [symbol] _AT, 211661_X_AT, 239 [symbol] 1 [symbol] _X_AT, 203 [symbol] 7_S_AT, 2276 [symbol] 7_AT, |
| | 205067_AT, 1555759_A_AT, 220066_AT |
| GOTERM_{—} | 210495_X_AT, 2164 [symbol] 2_X_AT, 229221_AT, 212171_X_AT, 212014_X_AT, 211719_X_AT, 221731_X_AT, |
| MF_ALL | 210916_S_AT, 1557 [symbol] 05_S_AT, 201548_S_AT, 22 [symbol] 1 [symbol] 6_S_AT, 210512_S_AT, 220066_AT, 211527_X_AT, |
| | 210 [symbol] 39_S_AT, 2044 [symbol] 9_S_AT, 20 [symbol] 490_S_AT, 204620_S_AT, 215646_S_AT, 20 [symbol] 619_S_AT, 210513_S_AT, |
| | 209 [symbol] 35_X_AT, 212190_AT, 206157_AT, 209192_AT, 211571_S_AT, 212464_S_AT |
| GOTERM_{—} | 210495_X_AT, 216442_X_AT, 229221_AT, 212171_X_AT, 212014_X_AT, 211719_X_AT, 221731_X_AT, |
| MF_ALL | 210916_S_AT, 1557905_S_AT, 20354 [symbol] _S_AT, 22 [symbol] 1 [symbol] 6_S_AT, 210512_S_AT, 220066_AT, 211527_X_AT, |
| | 210 [symbol] 39_S_AT, 2044 [symbol] _S_AT, 204490_S_AT, 204620_S_AT, 215646_S_AT, 204619_S_AT, 210513_S_AT, |
| | 20 [symbol] 35_X_AT, 212190_AT, 206157_AT, 209392_AT, 211571_S_AT, 212464_S_AT |
| GOTERM_{—} | 201464_X_AT, 201465_S_AT, 37152_AT, 212298_AT, 219028_AT, 221841_S_AT, 204923_AT, |
| BP_ALL | 213763_AT, 205067_AT, 1566901_AT, 202 [symbol] 59_X_AT, 2022 [symbol] 4_S_AT, 236561_AT, 203729_AT, |
| | 203665_AT, 239519_AT, 201565_S_AT, 225116_AT, 223502_S_AT, 201466_S_AT, 212143_S_AT, |
| | 1552914_A_AT, 204035_AT, 225115_AT, 212124_AT, 225368_AT, 201005_AT, 224 [symbol] 59_AT, |
| | 210095_S_AT, 201012_AT, 214211_AT, 212171_X_AT, 39402_AT, 205463_S_AT, 201566_X_AT, |
| | 223501_AT, 22 [symbol] 30_AT, 200748_S_AT, 239818_X_AT, 203940_S_AT, 200921_S_AT, 210512_S_AT, |
| | 200920_S_AT, 211527_X_AT, 203140_AT, 220266_S_AT, 202241_AT, 213281_AT, 210513_S_AT, |
| | 23 [symbol] 669_AT, 225097_AT, 219257_S_AT, 223394_AT |
| GOTERM_{—} | 213891_S_AT, 201464_X_AT, 201465_S_AT, 219028_AT, 221841_S_AT, 201473_AT, 242794_AT, |
| BP_ALL | 211352_S_AT, 21294 [symbol] _AT, 201471_S_AT, 235739_AT, 20 [symbol] 960_S_AT, 213763_AT, 205067_AT, |
| | 236561_AT, 220066_AT, 200 [symbol] 7 [symbol] _AT, 205 [symbol] 91_AT, 20 [symbol] 665_AT, 20375 [symbol] _AT, 225116_AT, 206157_AT, |
| | 201466_S_AT, 1552914_A_AT, 207700_S_AT, 225115_AT, 1555832_S_AT, 222146_S_AT, 212124_AT, |
| | 225368_AT, 205312_AT, 20 [symbol] 961_S_AT, 224859_AT, 216248_S_AT, 212171_X_AT, 39402_AT, |
| | 204622_X_AT, 205463_S_AT, 235457_AT, 2123 [symbol] 7_AT, 229 [symbol] 30_AT, 21 [symbol] 559_S_AT, 222670_S_AT, |
| | 210512_S_AT, 211527_X_AT, 244414_AT, 242405_AT, 220266_S_AT, 2132 [symbol] 1_AT, 224606_AT, |
| | 209189_AT, 210513_S_AT, 203935_AT, 212386_AT, 225097_AT, 225557_AT, 223394_AT |
| GOTERM_{—} | 1554992_AT, 203665_AT, 205 [symbol] 91_AT, 39402_AT, 21 [symbol] 34_S_AT, 238669_AT, 15565 [symbol] 3_A_AT, |
| BP_ALL | 240 [symbol] 65_AT, 206171_AT, 219257_S_AT, 205067_AT, 202071_AT, 200 [symbol] 78_AT, 201170_S_AT, |
| | 2156 [symbol] 8_AT, 1562467_AT |
| GOTERM_{—} | 203 [symbol] 65_AT, 205891_AT, 203140_AT, 222062_AT, 39402_AT, 217757_AT, 205067_AT, 220066_AT, |
| BP_ALL | 226218_AT, 204923_AT |
| GOTERM_{—} | 202540_S_AT, 213891_S_AT, 201464_X_AT, 201465_S_AT, 212171_X_AT, 216248_S_AT, 209659_AT, |
| MF_ALL | 205463_S_AT, 204622_X_AT, 241722_X_AT, 20 [symbol] 923_S_AT, 201473_AT, 2123 [symbol] 7_AT, 229 [symbol] 30_AT, |
| | 227107_AT, 235739_AT, 210512_S_AT, 236561_AT, 211527_X_AT, 200878_AT, 20 [symbol] 751_X_AT, |
| | 1562467_AT, 20499 [symbol] _S_AT, 202768_AT, 2132 [symbol] 1_AT, 221840_AT, 210513_S_AT, 1552553_A_AT, |
| | 20918 [symbol] _AT, 221012_S_AT, 203753_AT, 203922_S_AT, 204 [symbol] 53_AT, 204715_AT, 201466_S_AT, |
| | 2 [symbol] 5295_AT, 203104_AT, 203935_AT, 201313_AT, 201995_AT, 2123 [symbol] 6_AT, 222146_S_AT, |
| | 203752_S_AT, 202539_S_AT |
| GOTERM_{—} | 212171_X_AT, 212298_AT, 63825_AT, 205463_S_AT, 229 [symbol] 30_AT, 205566_AT, 203940_S_AT, |
| BP_ALL | 2 [symbol] 1 [symbol] _X_AT, 236561_AT, 210512_S_AT, [symbol] 7100_AT, 231527_X_AT, 210839_S_AT, 22 [symbol] 490_AT, |
| | 203665_AT, 203140_AT, 202241_AT, 210513_S_AT, 239519_AT, 212190_AT, 221 [symbol] 15_AT, 225337_AT, |
| | 212343_S_AT, 219257_S_AT, 209392_AT, 210095_S_AT |
| GOTERM_{—} | 201464_X_AT, 201465_S_AT, 21902 [symbol] _AT, 212171_X_AT, 2122 [symbol] _AT, 205463_S_AT, 229 [symbol] 30_AT, |
| BP_ALL | 213763_AT, 2026 [symbol] 6_S_AT, 226389_S_AT, 236561_AT, 210512_S_AT, 211527_X_AT, 221 [symbol] 40_AT, |
| | 2132 [symbol] 1_AT, 2091 [symbol] 9_AT, 210513_S_AT, 239519_AT, 214054_AT, 203037_S_AT, 229116_AT, |
| | 225738_AT, 2014 [symbol] 6_S_AT, 203935_AT, 203104_AT, 225115_AT, 225097_AT, 22536 [symbol] _AT, |
| | 20 [symbol] 37_S_AT, 225557_AT, 210173_AT |
| GOTERM_{—} | 229221_AT, 214297_AT, 212171_X_AT, 209555_S_AT, 205463_S_AT, 212014_X_AT, 211924_S_AT, |
| CC_ALL | 214 [symbol] 66_AT, 226218_AT, 210916_S_AT, 229830_AT, 209994_S_AT, 206488_S_AT, 1557905_S_AT, |
| | 23 [symbol] 542_AT, 210512_S_AT, 220066_AT, 211527_X_AT, 202071_AT, 2044 [symbol] 9_S_AT, 204736_S_AT, |
| | 204490_S_AT, 220307_AT, 210513_S_AT, 209 [symbol] 35_X_AT, 1552914_A_AT, 204105_S_AT, 202756_S_AT, |
| | 22 [symbol] 766_AT, 241929_AT, 210 [symbol] 45_S_AT, 224859_AT, 209993_AT |
| GOTERM_{—} | 214211_AT, 201464_X_AT, 223723_AT, 201465_S_AT, 37152_AT, 210136_AT, 39402_AT, |
| BP_ALL | 241722_X_AT, 1405_I_AT, 241938_AT, 204661_AT, 34210_AT, 205099_S_AT, 200748_S_AT, |
| | 20 [symbol] 01_AT, 205067_AT, 201925_S_AT, 1562 [symbol] 67_AT, 209906_AT, 216017_S_AT, 213281_AT, |
| | 204655_AT, 201466_S_AT, 239451_AT, 15565 [symbol] 3_A_AT, 212803_AT, 1555759_A_AT, 241773_AT, |
| | 201005_AT, 205098_AT |
| GOTERM_{—} | 202540_S_AT, 37152_AT, 21902 [symbol] _AT, 2279 [symbol] _AT, 216834_AT, 20247 [symbol] _AT, 211352_S_AT, |
| BP_ALL | 201471_S_AT, 213763_AT, 24 [symbol] 0_S_AT, 2263 [symbol] 9_S_AT, 205067_AT, 1566901_AT, 236561_AT, |
| | 220066_AT, 202 [symbol] _AT, 205891_AT, 203 [symbol] 5_AT, 20 [symbol] 891_AT, 225116_AT, 212143_S_AT, |
| | 207700_S_AT, 225115_AT, 215602_AT, 22536 [symbol] _AT, 202 [symbol] _S_AT, 241773_AT, 243556_AT, |
| | 2100 [symbol] 5_S_AT, 20253 [symbol] _S_AT, 212171_X_AT, 218501_AT, 39402_AT, 227240_AT, 22339 [symbol] _AT, |
| | 239818_X_AT, 208893_S_AT, 219 [symbol] 45_AT, 219908_AT, 210512_S_AT, 2191 [symbol] 3_S_AT, 211527_X_AT, |
| | 1554992_AT, 203140_AT, 20 [symbol] 92_S_AT, 202241_AT, 210513_S_AT, 1565754_X_AT, 22573 [symbol] _AT, |
| | 1565752_AT, 20 [symbol] 935_AT, 210258_AT, 227697_AT, 225097_AT, 217591_AT, 2156 [symbol] _AT |
| GOTERM_{—} | 212298_AT, 212171_X_AT, 63825_AT, 209555_S_AT, 39402_AT, 205463_S_AT, 228708_AT, |
| BP_ALL | 229830_AT, 227107_AT, 205566_AT, 204401_AT, 2064 [symbol] _S_AT, 239 [symbol] 1 [symbol] _X_AT, 203940_S_AT, |
| | 205067_AT, 203548_S_AT, 210512_S_AT, 236561_AT, 220066_AT, [symbol] 7100_AT, 211527_X_AT, |
| | 210 [symbol] 39_S_AT, 228490_AT, 203665_AT, 205 [symbol] 91_AT, 203140_AT, 202241_AT, 239519_AT, |
| | 210513_S_AT, 212190_AT, 204715_AT, 221 [symbol] 15_AT, 206157_AT, 212143_S_AT, 225337_AT, |
| | 235669_AT, 235295_AT, 1556583_A_AT, 228766_AT, 219257_S_AT, 209392_AT, 241929_AT, |
| | 210095_S_AT |
| GOTERM_{—} | 203665_AT, 203140_AT, 203233_AT, 227062_AT, 217757_AT, 220066_AT, 22621 [symbol] _AT, 224 [symbol] 59_AT, |
| BP_ALL | 1552914_A_AT |
| GOTERM_{—} | 217173_S_AT, 37152_AT, 219155_AT, 209555_S_AT, 23 [symbol] 649_AT, 202068_S_AT, 212062_AT, |
| BP_ALL | 204401_AT, 226350_AT, 206 [symbol] _S_AT, 22 [symbol] 766_AT, 214255_AT, 226302_AT, 241929_AT, |
| | 202067_S_AT, 201012_AT, 209122_AT |
| GOTERM_{—} | 227069_AT, 229221_AT, 37152_AT, 212298_AT, 210136_AT, 212014_X_AT, 221731_X_AT, 222 [symbol] 77_AT, |
| BP_ALL | 241938_AT, 210916_S_AT, 235739_AT, 202 [symbol] 28_S_AT, 1566901_AT, 230360_AT, 236561_AT, |
| | 204141_AT, 204489_S_AT, 216017_S_AT, 204490_S_AT, 204998_S_AT, 20 [symbol] 620_S_AT, 201149_S_AT, |
| | 239519_AT, 201147_S_AT, 209 [symbol] 35_X_AT, 232649_AT, 204653_AT, 212190_AT, 2204 [symbol] 4_AT, |
| | 201005_AT, 243556_AT, 2119 [symbol] 6_AT, 233220_AT, 160020_AT, 212172_X_AT, 21624 [symbol] _S_AT, |
| | 204622_X_AT, 227240_AT, 202191_S_AT, 218559_S_AT, 1557905_S_AT, 204684_AT, 210512_S_AT, |
| | 202 [symbol] 27_S_AT, 222670_S_AT, 211527_X_AT, 201150_S_AT, 204465_S_AT, 201248_S_AT, 1554 [symbol] 2_AT, |
| | 220935_S_AT, 210512_S_AT, 2091 [symbol] 9_AT, 215646_S_AT, 204619_S_AT, 212372_AT, 204105_S_AT, |
| | 229797_AT, 212 [symbol] 03_AT, 217279_X_AT, 215 [symbol] 8_AT, 211571_S_AT |
| GOTERM_{—} | 2014 [symbol] 4_X_AT, 201465_S_AT, 37152_AT, 210136_AT, 2122 [symbol] _AT, 2162 [symbol] _S_AT, 39402_AT, |
| BP_ALL | 205463_S_AT, 204622_X_AT, 1405_I_AT, 241938_AT, 229 [symbol] 30_AT, 205099_S_AT, 227107_AT, |
| | 235739_AT, 2026 [symbol] _S_AT, 2046 [symbol] 4_AT, 219845_AT, 205067_AT, 2022 [symbol] 4_S_AT, 221463_AT, |
| | 1562467_AT, 21 [symbol] 017_S_AT, 203665_AT, 205 [symbol] 1_AT, 213281_AT, 204655_AT, 239519_AT, 2091 [symbol] 9_AT, |
| | 204715_AT, 201466_S_AT, 235295_AT, 15565 [symbol] 3_A_AT, 212 [symbol] 03_AT, 1555759_A_AT, 210173_AT, |
| | 201005_AT, 20509 [symbol] _AT |
| GOTERM_{—} | 212171_X_AT, 212124_AT, 211962_S_AT, 210513_S_AT, 210512_S_AT, 201473_AT, 210173_AT, |
| BP_ALL | 211527_X_AT, 24193 [symbol] _AT |
| GOTERM_{—} | 201464_X_AT, 201465_S_AT, 37152_AT, 219028_AT, 212171_X_AT, 212298_AT, 39402_AT, |
| BP_ALL | 205463_S_AT, 201566_X_AT, 223501_AT, 229 [symbol] 30_AT, 204923_AT, 213763_AT, 1566 [symbol] 01_AT, |
| | 205067_AT, 236561_AT, 2022 [symbol] 4_S_AT, 210512_S_AT, 211527_X_AT, 203140_AT, 213281_AT, |
| | 239519_AT, 201565_S_AT, 210513_S_AT, 225116_AT, 223502_S_AT, 201466_S_AT, 1552914_A_AT, |
| | 204035_AT, 225115_AT, 225097_AT, 212124_AT, 219257_S_AT, 225368_AT, 224859_AT, 223394_AT |
| GOTERM_{—} | 214211_AT, 201464_X_AT, 201465_S_AT, 37152_AT, 39402_AT, 221842_S_AT, 20074 [symbol] _S_AT, |
| BP_ALL | 203940_S_AT, 23981 [symbol] _X_AT, 200921_S_AT, 1566901_AT, 205067_AT, 202284_S_AT, 202859_X_AT, |
| | 200920_S_AT, 203729_AT, 203665_AT, 203140_AT, 220266_S_AT, 213281_AT, 202241_AT, |
| | 201466_S_AT, 1552914_A_AT, 212143_S_AT, 204035_AT, 201005_AT, 224859_AT, 210095_S_AT |
| GOTERM_{—} | 201464_X_AT, 201465_S_AT, 229221_AT, 21902 [symbol] _AT, 227948_AT, 212014_X_AT, 210916_S_AT, |
| BP_ALL | 201041_S_AT, 201471_S_AT, 235739_AT, 213763_AT, 217997_AT, 205067_AT, 202284_S_AT, |
| | 236561_AT, 220066_AT, 204489_S_AT, 203665_AT, 204490_S_AT, 20499 [symbol] _S_AT, 201149_S_AT, |
| | 201147_S_AT, 209835_X_AT, 225116_AT, 223502_S_AT, 201466_S_AT, 212143_S_AT, 204035_AT, |
| | 225115_AT, 215602_AT, 225368_AT, 243556_AT, 210095_S_AT, 205681_AT, 201012_AT, |
| | 212171_X_AT, 216248_S_AT, 39402_AT, 21 [symbol] 501_AT, 204622_X_AT, 243797_AT, 241722_X_AT, |
| | 227240_AT, 223501_AT, 2020 [symbol] 6_AT, 1557905_S_AT, 200921_S_AT, 210512_S_AT, 200920_S_AT, |
| | 211527_X_AT, 201150_S_AT, 217996_AT, 201148_S_AT, 1554992_AT, 203140_AT, 213281_AT, |
| | 210513_S_AT, 1552553_A_AT, 1565754_X_AT, 201044_X_AT, 225 [symbol] 42_AT, 1565752_AT, 203 [symbol] 35_AT, |
| | 239451_AT, 227697_AT, 225097_AT, 219257_S_AT, 2156 [symbol] _AT |
| GOTERM_{—} | 205 [symbol] 91_AT, 203140_AT, 203233_AT, 39402_AT, 205067_AT, 2022 [symbol] 4_S_AT, 226218_AT, 223501_AT, |
| BP_ALL | 224 [symbol] 59_AT, 223502_S_AT, 1552914_A_AT, 20 [symbol] 923_AT |
| GOTERM | 203140_AT, 205891_AT, 203665_AT, 39402_AT, 22621 [symbol] _AT, 223501_AT, 223502_S_AT, 20 [symbol] 923_AT, |
| BP_ALL | 1552914_A_AT, 203233_AT, 222062_AT, 205067_AT, 2022 [symbol] 4_S_AT, 224 [symbol] 59_AT |
| GOTERM_{—} | 216442_X_AT, 210495_X_AT, 212271_X_AT, 22794 [symbol] _AT, 210513_S_AT, 211719_X_AT, 1565754_X_AT, |
| BP_ALL | 202191_S_AT, 212372_AT, 1565752_AT, 215602_AT, 214255_AT, 210512_S_AT, 211527_X_AT, |
| | 212464_S_AT |
| GOTERM_{—} | 213391_S_AT, 201454_X_AT, 201465_S_AT, 219028_AT, 221 [symbol] 41_S_AT, 201473_AT, 242794_AT |
| BP_ALL | 211352_S_AT, 212948_AT, 201471_S_AT, 235739_AT, 20 [symbol] 960_S_AT, 213763_AT, 205067_AT |
| | 1554453_AT, 236561_AT, 200 [symbol] 78_AT, 205891_AT, 203753_AT, 225116_AT, 206157_AT, 201466_S_AT |
| | 207700_S_AT, 225115_AT, 1555832_S_AT, 222146_S_AT, 212124_AT, 225368_AT, 205312_AT, |
| | 20 [symbol] 961_S_AT, 216248_S_AT, 212171_X_AT, 39402_AT, 205463_S_AT, 204622_X_AT, 235457_AT, |
| | 2123 [symbol] 7_AT, 229 [symbol] 30_AT, 218559_S_AT, 222670_S_AT, 210512_S_AT, 211527_X_AT, 244414_AT, |
| | 242405_AT, 220266_S_AT, 213281_AT, 224606_AT, 209189_AT, 210513_S_AT, 203935_AT, |
| | 212386_AT, 225097_AT, 225557_AT, 223394_AT |
| GOTERM_{—} | 201464_X_AT, 201455_S_AT, 229221_AT, 219028_AT, 227946_AT, 212014_X_AT, 210916_S_AT, |
| BP_ALL | 201041_S_AT, 201471_S_AT, 235739_AT, 213763_AT, 217997_AT, 205067_AT, 2022 [symbol] 4_S_AT, |
| | 23 [symbol] 561_AT, 220066_AT, 2044 [symbol] 9_S_AT, 203665_AT, 204490_S_AT, 20499 [symbol] _S_AT, 201149_S_AT, |
| | 201147_S_AT, 209835_X_AT, 225116_AT, 223502_S_AT, 201466_S_AT, 212143_S_AT, 204035_AT, |
| | 225115_AT, 215602_AT, 225368_AT, 243556_AT, 210095_S_AT, 2056 [symbol] 1_AT, 201012_AT, |
| | 212171_X_AT, 21624 [symbol] _S_AT, 39402_AT, 21 [symbol] 501_AT, 204622_X_AT, 243797_AT, 241722_X_AT, |
| | 227240_AT, 223501_AT, 202 [symbol] 6_AT, 1557905_S_AT, 200922_S_AT, 210512_S_AT, 200920_S_AT, |
| | 211527_X_AT, 201150_S_AT, 217996_AT, 201148_S_AT, 1554992_AT, 203140_AT, 213281_AT, |
| | 210513_S_AT, 1552553_A_AT, 1565754_X_AT, 201044_X_AT, 225842_AT, 1565752_AT, 203935_AT, |
| | 239451_AT, 227697_AT, 225097_AT, 219257_S_AT, 215688_AT |
| GOTERM_{—} | 201464_X_AT, 201465_S_AT, 229221_AT, 219028_AT, 227948_AT, 212014_X_AT, 210916_S_AT, |
| BP_ALL | 201041_S_AT, 201471_S_AT, 235739_AT, 213763_AT, 217997_AT, 205067_AT, 2022 [symbol] 4_S_AT, |
| | 236561_AT, 220066_AT, 2044 [symbol] 9_S_AT, 203665_AT, 204490_S_AT, 20499 [symbol] _S_AT, 201149_S_AT, |
| | 201147_S_AT, 209835_X_AT, 225116_AT, 223502_S_AT, 201466_S_AT, 212143_S_AT, 204035_AT, |
| | 225115_AT, 215602_AT, 225368_AT, 243556_AT, 210095_S_AT, 205681_AT, 201012_AT, |
| | 212171_X_AT, 21624 [symbol] _S_AT, 39402_AT, 21 [symbol] 501_AT, 204622_X_AT, 243797_AT, 241722_X_AT, |
| | 227240_AT, 223501_AT, 2020 [symbol] 6_AT, 1557905_S_AT, 200921_S_AT, 210512_S_AT, 200920_S_AT, |
| | 211527_X_AT, 201150_S_AT, 217996_AT, 201248_S_AT, 1554992_AT, 203140_AT, 2132 [symbol] 1_AT, |
| | 210513_S_AT, 1552553_A_AT, 1565754_X_AT, 201044_X_AT, 225842_AT, 1565752_AT, 203935_AT, |
| | 239451_AT, 227697_AT, 225097_AT, 219257_S_AT, 2156 [symbol] _AT |
| GOTERM_{—} | 205891_AT, 203140_AT, 217757_AT, 203508_AT, 204 [symbol] 55_AT, 1405_I_AT, 1555759_A_AT, 224859_AT, |
| BP_ALL | 203939_AT, 1552914_A_AT |
| GOTERM_{—} | 204035_AT, 212171_X_AT, 210513_S_AT, 210512_S_AT, 202859_X_AT, 211527_X_AT, 20 [symbol] 27_S_AT |
| BP_ALL | |
| GOTERM_{—} | 202540_S_AT, 37152_AT, 215028_AT, 2279 [symbol] _AT, 216834_AT, 202478_AT, 211352_S_AT, |
| BP_ALL | 201471_S_AT, 213763_AT, 224480_S_AT, 226389_S_AT, 205067_AT, 15 [symbol] 6901_AT, 236561_AT, |
| | 2200 [symbol] 6_AT, 201170_S_AT, 2023 [symbol] _AT, 203 [symbol] 65_AT, 2058 [symbol] 1_AT, 20 [symbol] 91_AT, 225116_AT, |
| | 212143_S_AT, 207700_S_AT, 225115_AT, 213602_AT, 22536 [symbol] _AT, 2029 [symbol] _S_AT, 241773_AT, |
| | 243556_AT, 210095_S_AT, 202539_S_AT, 212171_X_AT, 218501_AT, 39402_AT, 227240_AT, |
| | 2232 [symbol] _AT, 239 [symbol] 15_X_AT, 20 [symbol] 93_S_AT, 219845_AT, 219908_AT, 210512_S_AT, 219183_S_AT, |
| | 211527_X_AT, 1562467_AT, 1554992_AT, 203140_AT, 20 [symbol] 92_S_AT, 202241_AT, 210513_S_AT, |
| | 1565754_X_AT, 22573 [symbol] _AT, 1565752_AT, 203935_AT, 210258_AT, 227697_AT, 225097_AT, |
| | 217591_AT, 2156 [symbol] _AT |
| GOTERM_{—} | 205891_AT, 203140_AT, 203233_AT, 39402_AT, 205067_AT, 202284_S_AT, 226218_AT, 223501_AT, |
| BP_ALL | 224859_AT, 223502_S_AT, 1552914_A_AT, 204923_AT |
| GOTERM_{—} | 213503_X_AT, 21902 [symbol] _AT, 39402_AT, 205463_S_AT, 22794 [symbol] _AT, 1405_I_AT, 210427_X_AT, |
| BP_ALL | 229830_AT, 213763_AT, 205067_AT, 220066_AT, 20 [symbol] 436_S_AT, 203939_AT, 203140_AT, 205 [symbol] 91_AT, |
| | 217757_AT, 20 [symbol] 16_X_AT, 204655_AT, 201590_X_AT, 225116_AT, 1552914_A_AT, 225115_AT, |
| | 225097_AT, 203508_AT, 225368_AT, 1555759_A_AT, 234 [symbol] 59_AT |
| GOTERM_{—} | 160020_AT, 216017_S_AT, 201069_AT, 21350 [symbol] _X_AT, 210427_X_AT, 201590_X_AT, 22421 [symbol] _S_AT, |
| BP_ALL | 204 [symbol] 3_AT, 212143_S_AT, 206 [symbol] 35_AT, 218502_S_AT, 201995_AT, 203752_S_AT, 202 [symbol] 28_S_AT, |
| | 212 [symbol] 03_AT, 236561_AT, 202827_S_AT, 223092_AT, 225557_AT, 217279_AT, 222651_S_AT, |
| | 2100 [symbol] 5_S_AT, 1569149_AT, 203751_X_AT |
| GOTERM_{—} | 203 [symbol] _AT, 214453_S_AT, 20 [symbol] 655_AT, 1405_I_AT, 1555759_A_AT, 20 [symbol] 436_S_AT, 211676_S_AT, |
| BP_ALL | 202086_AT, 229 [symbol] 37_X_AT, 208438_S_AT, 213293_S_AT |
| GOTERM_{—} | 201464_X_AT, 201465_S_AT, 229221_AT, 37152_AT, 212171_X_AT, 224967_AT, 205463_S_AT, |
| BP_ALL | 221841_S_AT, 212014_X_AT, 211924_S_AT, 214 [symbol] 66_AT, 201473_AT, 222877_AT, 210916_S_AT, |
| | 229 [symbol] 30_AT, 1557905_S_AT, 206 [symbol] 35_AT, 219 [symbol] 45_AT, 1566901_AT, 210512_S_AT, 211527_X_AT, |
| | 1562467_AT, 201150_S_AT, 204465_S_AT, 201148_S_AT, 2044 [symbol] 9_S_AT, 204490_S_AT, 220266_S_AT, |
| | 2132 [symbol] 1_AT, 201149_S_AT, 210 [symbol] 13_S_AT, 201147_S_AT, 209 [symbol] 35_X_AT, 204653_AT, 201466_S_AT, |
| | 212372_AT, 203935_AT, 204881_S_AT, 201995_AT, 21 [symbol] 45_S_AT, 201324_AT, 201012_AT |
| GOTERM_{—} | 214211_AT, 209906_AT, 223723_AT, 203665_AT, 39402_AT, 1405_I_AT, 204655_AT, 34230_AT, |
| BP_ALL | 204661_AT, 20 [symbol] 099_S_AT, 20074 [symbol] _S_AT, 239451_AT, 15565 [symbol] 3_A_AT, 205067_AT, 1555759_A_AT, |
| | 241773_AT, 201925_S_AT, 20509 [symbol] _AT |
| GOTERM_{—} | 216442_X_AT, 210 [symbol] 95_X_AT, 21229 [symbol] _AT, 210136_AT, 212171_X_AT, 22794 [symbol] _AT, 23 [symbol] 519_AT, |
| BP_ALL | 210513_S_AT, 211719_X_AT, 1565754_X_AT, 202191_S_AT, 212372_AT, 1565752_AT, 204105_S_AT, |
| | 215602_AT, 214255_AT, 210512_S_AT, 211527_X_AT, 22464_S_AT |
| GOTERM_{—} | 201464_X_AT, 201465_S_AT, 39402_AT, 20546 [symbol] _S_AT, 2112 [symbol] 1_AT, 1405_I_AT, 204655_AT, |
| BP_ALL | 2091 [symbol] 9_AT, 201473_AT, 229 [symbol] 30_AT, 201 [symbol] 66_S_AT, 20 [symbol] 4_S_AT, 201315_AT, 227697_AT, |
| | 155658 [symbol] _A_AT, 205067_AT, 2022 [symbol] 4_S_AT, 1555759_A_AT, 20 [symbol] 2_AT, 207 [symbol] 19_S_AT, 209122_AT |
| GOTERM_{—} | 160020_AT, 229221_AT, 227 [symbol] 9_AT, 212171_X_AT, 21229 [symbol] _AT, 205463_S_AT, 212014_X_AT, |
| BP_ALL | 210916_S_AT, 219 [symbol] 30_AT, 1557905_S_AT, 202 [symbol] 2 [symbol] _S_AT, 1566 [symbol] 01_AT, 236561_AT, 210512_S_AT, |
| | 202 [symbol] 27_S_AT, 211527_X_AT, 200 [symbol] 7 [symbol] _AT, 204 [symbol] 9_S_AT, 204490_S_AT, 210513_S_AT, 235519_AT, |
| | 20 [symbol] 35_X_AT, 204653_AT, 203935_AT, 217279_X_AT, 203529_AT |
| GOTERM_{—} | 219028_AT, 212171_X_AT, 216248_S_AT, 39402_AT, 204622_X_AT, 241722_X_AT, 223501_AT, |
| BP_ALL | 201471_S_AT, 235739_AT, 213763_AT, 205067_AT, 236561_AT, 202284_S_AT, 210512_S_AT, |
| | 211527_X_AT, 203140_AT, 203665_AT, 20499 [symbol] _S_AT, 210513_S_AT, 225116_AT, 223502_S_AT, |
| | 204035_AT, 203935_AT, 225115_AT, 239451_AT, 227697_AT, 225097_AT, 219257_S_AT, 22536 [symbol] _AT, |
| | 201012_AT, 205681_AT |
| GOTERM_{—} | 212171_X_AT, 39402_AT, 216243_S_AT, 1405_I_AT, 204655_AT, 210513_S_AT, 223501_AT, |
| MF_ALL | 223502_S_AT, 204035_AT, 212657_S_AT, 205067_AT, 210512_S_AT, 1555759_A_AT, 202859_X_AT, |
| | 221463_AT, 211527_X_AT, 209827_S_AT |
| GOTERM_{—} | 205463_S_AT, 212124_AT, 219257_S_AT, 1566901_AT, 2022 [symbol] 4_S_AT, 229 [symbol] 30_AT |
| BP_ALL | |
| GOTERM_{—} | 20114 [symbol] _S_AT, 216442_X_AT, 210495_X_AT, 21342 [symbol] _S_AT, 212171_X_AT, 213503_X_AT, |
| CC_ALL | 20 [symbol] 16_X_AT, 201149_S_AT, 210513_S_AT, 210427_X_AT, 201147_S_AT, 201590_X_AT, 211719_X_AT, |
| | 235944_AT, 210512_S_AT, 211527_X_AT, 201150_S_AT, 212464_S_AT |
| KEGG_{—} | 237252_AT, 209906_AT, 203 [symbol] 87_S_AT, 217757_AT, 211924_S_AT, 217552_X_AT, 214866_AT, |
| PATHWAY | 210845_S_AT, 201925_S_AT, 203 [symbol] _AT |
| GOTERM_{—} | 20114 [symbol] _S_AT, 21 [symbol] 442_X_AT, 210495_X_AT, 212171_X_AT, 213503_X_AT, 208816_X_AT, |
| CC_ALL | 201149_S_AT, 210513_S_AT, 210427_X_AT, 201147_S_AT, 201590_X_AT, 211719_X_AT, 235944_AT, |
| | 210512_S_AT, 211527_X_AT, 201150_S_AT, 212464_S_AT |
| GOTERM_{—} | 219028_AT, 212171_X_AT, 216248_S_AT, 39402_AT, 204622_X_AT, 241722_X_AT, 223501_AT, |
| BP_ALL | 201471_S_AT, 235739_AT, 213763_AT, 205067_AT, 236561_AT, 2022 [symbol] 4_S_AT, 210512_S_AT, |
| | 211527_X_AT, 203140_AT, 203665_AT, 2049 [symbol] _S_AT, 210513_S_AT, 225116_AT, 223502_S_AT, |
| | 204035_AT, 203935_AT, 225115_AT, 239451_AT, 227697_AT, 225097_AT, 219257_S_AT, 225368_AT, |
| | 201012_AT, 205681_AT |
| GOTERM_{—} | 21902 [symbol] _AT, 212171_X_AT, 216248_S_AT_S_AT, 39402_AT, 204622_X_AT, 241722_X_AT, 223501_AT, |
| BP_ALL | 201471_S_AT, 235739_AT, 213763_AT, 205067_AT, 236561_AT, 202284_S_AT, 210512_S_AT, |
| | 211527_X_AT, 203140_AT, 203665_AT, 204998_S_AT, 210513_S_AT, 225116_AT, 223502_S_AT, |
| | 204035_AT, 203935_AT, 225115_AT, 239451_AT, 227697_AT, 225097_AT, 219257_S_AT, 225368_AT, |
| | 201012_AT, 2056 [symbol] 1_AT |
| GOTERM_{—} | 203140_AT, 39402_AT, 205067_AT, 202284_S_AT, 223501_AT, 224859_AT, 223502_S_AT, |
| BP_ALL | 155291_A_AT, 204923_AT |
| GOTERM_{—} | 203140_AT, 39402_AT, 205067_AT, 2022 [symbol] _S_AT, 223501_AT, 224 [symbol] 59_AT, 223502_S_AT, |
| BP_ALL | 1552914_A_AT, 204923_AT |
| GOTERM_{—} | 203140_AT, 39402_AT, 205067_AT, 2022 [symbol] 4_S_AT, 223501_AT, 224 [symbol] 59_AT, 223502_S_AT, |
| BP_ALL | 1552914_A_AT, 204923_AT |
| GOTERM_{—} | 216442_X_AT, 210495_X_AT, 212171_X_AT, 39402_AT, 209555_S_AT, 217757_AT, 205463_S_AT, |
| CC_ALL | 228708_AT, 210513_S_AT, 209549_AT, 211719_X_AT, 229 [symbol] 30_AT, 2064 [symbol] 8_S_AT, 2046 [symbol] 4_AT, |
| | 22 [symbol] 766_AT, 205067_AT, 210512_S_AT, 241 [symbol] 29_AT, 202005_AT, 211527_X_AT, 212464_S_AT |
| GOTERM_{—} | 201464_X_AT, 213 [symbol] 91_S_AT, 201465_S_AT, 219028_AT, 221 [symbol] 41_S_AT, 201473_AT, 242794_AT, |
| BP_ALL | 211352_S_AT, 21294 [symbol] _AT, 201471_S_AT, 235739_AT, 20 [symbol] 960_S_AT, 213763_AT, 236561_AT, |
| | 200 [symbol] 7 [symbol] _AT, 201753_AT, 225116_AT, 201466_S_AT, 223502_S_AT, 207700_S_AT, 225115_AT, |
| | 1555 [symbol] 32_S_AT, 222146_S_AT, 212124_AT, 22536 [symbol] _AT, 205312_AT, 20 [symbol] 961_S_AT, 212171_X_AT, |
| | 21 [symbol] 2 [symbol] _S_AT, 204622_X_AT_, 235457_AT, 22 [symbol] 64_AT, 22350 [symbol] _AT, 2123 [symbol] 7_AT, 21 [symbol] 559_S_AT, |
| | 210512_S_AT, 212670_S_AT, 211527_X_AT, 24 [symbol] 14_AT, 220266_S_AT, 242405_AT, 2132 [symbol] 1_AT, |
| | 224606_AT, 210513_S_AT, 2091 [symbol] 9_AT, 203935_AT, 2123 [symbol] 6_AT, 225097_AT, 225557_AT, 2233 [symbol] 4_AT |
| GOTERM_{—} | 213 [symbol] 91_S_AT, 201464_X_AT, 201465_S_AT, 215028_AT, 221 [symbol] 41_S_AT, 201473_AT, 242794_AT, |
| BP_ALL | 211352_S_AT, 21294 [symbol] _AT, 201471_S_AT, 235739_AT, 20 [symbol] 960_S_AT, 213763_AT, 2050 [symbol] 7_AT, |
| | 236561_AT, 200 [symbol] 78_AT, 203665_AT, 203753_AT, 225116_AT, 201466_S_AT, 1552914_A_AT, |
| | 207700_S_AT, 225115_AT, 1555 [symbol] 32_S_AT, 222146_S_AT, 212124_AT, 2253 [symbol] _AT, 205312_AT, |
| | 20 [symbol] 961_S_AT, 224 [symbol] 59_AT, 21624 [symbol] _S_AT, 212171_X_AT, 39402_AT, 205463_S_AT, 204622_X_AT, |
| | 235457_AT, 212387_AT, 229 [symbol] 30_AT, 218559_S_AT, 222670_S_AT, 210512_S_AT, 211527_X_AT, |
| | 244414_AT, 242405_AT, 220266_S_AT, 2132 [symbol] 1_AT, 224606_AT, 2091 [symbol] 9_AT, 210513_S_AT, |
| | 203935_AT, 2123 [symbol] 6_AT, 225097_AT, 225557_AT, 223394_AT |
| GOTERM_{—} | 203665_AT, 203140_AT, 205891_AT, 213503_X_AT, 212171_X_AT, 205463_S_AT, 20 [symbol] 16_X_AT, |
| BP_ALL | 210427_X_AT, 210513_S_AT, 201590_X_AT, 229 [symbol] 30_AT, 206 [symbol] 35_AT, 210512_S_AT, 220066_AT, |
| | 211527_X_AT |
| GOTERM_{—} | 209906_AT, 39402_AT, 15565 [symbol] 3_A_AT, 205067_AT, 241773_AT, 201925_S_AT, 204661_AT, 34230_AT, |
| BP_ALL | 205099_S_AT, 20609 [symbol] _AT |
| GOTERM_{—} | 160020_AT, 201069_AT, 216017_S_AT, 212143_S_AT, 206 [symbol] 35_AT, 201995_AT, 203752_S_AT, |
| BP_ALL | 202 [symbol] 2 [symbol] _S_AT, 212803_AT, 202 [symbol] 27_S_AT, 217279_X_AT, 210095_S_AT, 203751_X_AT, 1569149_AT |

[symbol]

indicates data missing or illegible when filed

The gene expression patterns induced by Copaxone® and Polimunol treatment were compared directly. The differential expression results were filtered based on the variability in observed control treatment effects relative to previous studies, to obtain a conservative subset of highest-confidence probesets. These analyses found that 27 highest-confidence probesets, representing 21 distinct genes, differed significantly by adjusted p value in expression between the two treatments (14 probesets downregulated, 13 upregulated by Polimunol relative to Copaxone). The upregulated and downregulated probesets are illustrated in Table 13 and Table 14, respectively.

TABLE 13
Human monocyte study: probesets upregulated by Polimunol
relative to Copaxone ®

| ID | Gene | raw.FC | AveExpr | P.Value | adj.P.Val |
|---|---|---|---|---|---|
| 202436_s_at | CYP1B1 | 1.328778115 | 10.99251935 | 2.76E−15 | 1.51E−10 |
| 202435_s_at | CYP1B1 | 1.291947504 | 11.10690276 | 1.98E−11 | 2.70E−07 |
| 202434_s_at | CYP1B1 | 1.34300307 | 8.896124188 | 5.54E−10 | 3.79E−06 |
| 229055_at | GPR68 | 1.233303306 | 7.906128859 | 3.58E−09 | 1.50E−05 |
| 202437_s_at | CYP1B1 | 1.283470087 | 11.35502759 | 5.53E−09 | 1.96E−05 |
| 212665_at | TIPARP | 1.170151206 | 9.316307674 | 2.29E−07 | 0.000543566 |
| 1554966_a_at | DOC1 | 1.127401263 | 8.443050186 | 5.83E−07 | 0.001274168 |
| 204684_at | NPTX1 | 1.121904593 | 10.50026157 | 4.27E−06 | 0.00556324 |
| 229354_at | PDCD6 | 1.082379299 | 10.56416164 | 8.47E−06 | 0.009858651 |
| | /// AHRR | | | | |
| 206710_s_at | EPB41L3 | 1.17828072 | 6.683285506 | 3.11E−05 | 0.021766496 |
| 204135_at | DOC1 | 1.093724798 | 8.540804821 | 4.46E−05 | 0.028883673 |
| 1569690_at | CCDC36 | 1.140648392 | 5.595578016 | 5.04E−05 | 0.030714439 |
| 211067_s_at | GAS7 | 1.080052886 | 9.66677524 | 5.29E−05 | 0.031621345 |

TABLE 14
Human monocyte study: probesets downregulated by Polimunol
relative to Copaxone ®

| ID | Gene | raw.FC | AveExpr | P.Value | adj.P.Val |
|---|---|---|---|---|---|
| 230927_at | — | −1.171546521 | 6.290190151 | 2.87E−06 | 0.004124895 |
| 206170_at | ADRB2 | −1.142744253 | 8.033661912 | 7.30E−06 | 0.008675631 |
| 228826_at | RNF43 | −1.090334004 | 8.930697832 | 9.19E−06 | 0.010052917 |
| 1554676_at | PRG1 | −1.337462167 | 7.452885159 | 1.08E−05 | 0.010571079 |
| 1557359_at | LOC285758 | −1.113608349 | 9.441580738 | 1.43E−05 | 0.012763184 |
| 229934_at | — | −1.146000763 | 9.629181433 | 1.67E−05 | 0.013799705 |
| 244190_at | THAP5 | −1.235324953 | 3.96404755 | 1.70E−05 | 0.013885917 |
| 1556346_at | COTL1 | −1.23259647 | 9.131189047 | 1.97E−05 | 0.01563098 |
| 215111_s_at | TSC22D1 | −1.115773148 | 9.642583368 | 2.19E−05 | 0.016510136 |
| 235274_at | — | −1.142519223 | 7.012670757 | 2.86E−05 | 0.020344359 |
| 1558782_a_at | LOC644137 | −1.13456131 | 8.05846686 | 3.69E−05 | 0.024635148 |
| 201147_s_at | TIMP3 | −1.069861862 | 11.50473352 | 5.32E−05 | 0.031621345 |
| 224559_at | MALAT1 | −1.221208621 | 9.943150851 | 7.51E−05 | 0.039950998 |
| 210592_s_at | SAT1 | −1.20105501 | 9.927587911 | 8.05E−05 | 0.042336898 |

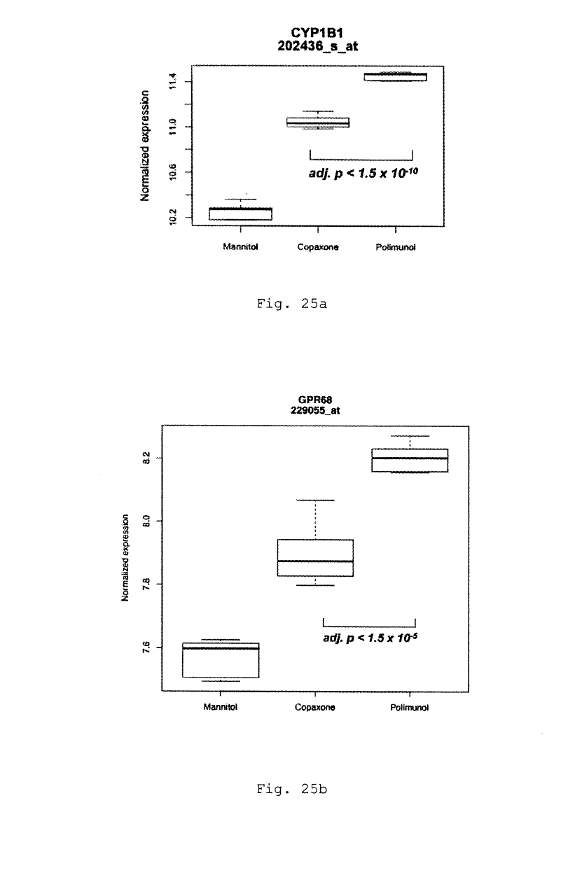

The gene with the highest fold change among those upregulated by Polimunol relative to Copaxone® was CYP1B1 (adj p<1.5e-10), a member of the cytochrome P450 superfamily of enzymes. All four present probesets for this gene in this study showed similar results, as exemplified in Table 13. As shown in FIG. 25a, CYP1B1 is significantly upregulated by Polimunol stimulation in human THP-1 monocyte cell line compared to Copaxone® stimulation at 6 hours. A second example is shown in Table 13 and FIG. 25b, a probeset for GPR68 which was also upregulated by Polimunol relative to Copaxone® (adj p<1.5e-5). Two probesets for the DOC1 gene were significantly upregulated by Polimunol relative to Copaxone, as shown in Table 13.

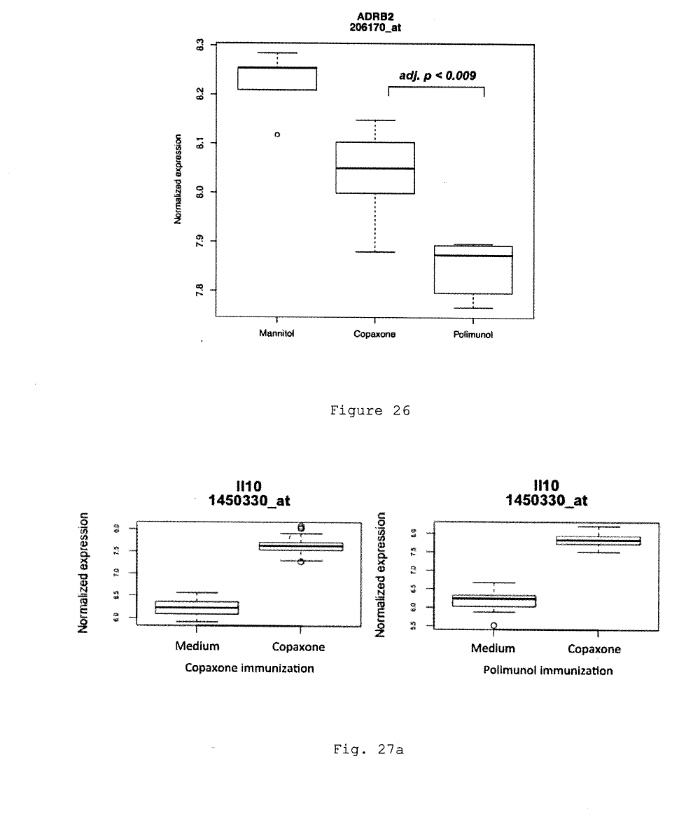

One of the probesets downregulated by Polimunol relative to Copaxone® was ADRB2, the gene encoding the beta-2 adrenergic receptor. As shown in FIG. 26, ADRB2 is significantly downregulated by Polimunol stimulation in human THP-1 monocyte cell line compared to Copaxone® stimulation at 6 hours.

### Discussion

### Example 9

Copaxone® treatment was compared with treatment with the purported generic Polimunol (manufactured by Synthon), as well as a mannitol control. More than 80% of the pathways that enriched among top upregulated genes in the previous study were also enriched among top upregulated genes in the current study. The results show concordance with the earlier study and confirm the gene expression patterns associated with Copaxone treatment of human monocytes—i.e. the complex, yet consistent mode of action of Copaxone® in this immunological cell type.

As an example at the individual gene level, IL1RN was significantly upregulated (adjusted p 2.8e-10), consistent with the previous study described in Example 1. This gene encodes for the protein IL-1ra, which inhibits the activities of pro-inflammatory cytokines IL-1a and IL-1b.

Overall, the results demonstrated that the effects of the complex biological mechanism of Copaxone® observed in prior studies were mechanistically consistent with the effects observed in this study, corroborating the validity of this approach for studying glatiramoid functionality in treatment paradigms.

The study also identified significant differences between Copaxone® and Synthon's Polimunol. The results show that 27 highest-confidence probesets, representing 21 distinct genes, differed significantly by adjusted p value in expression between the two treatments (14 probesets downregulated, 13 upregulated by Polimunol relative to Copaxone).

The gene with the highest fold change among those upregulated by Polimunol relative to Copaxone® was CYP1B1 (adj p<1.5e-10), a member of the cytochrome P450 superfamily of enzymes. The cytochrome P450 proteins are monooxygenases which catalyze many reactions involved in drug metabolism.

A second example is a probeset for GPR68 which was also upregulated by Polimunol relative to Copaxone® (adj p<1.5e-5). After traumatic brain injury, cerebral cortical astrocytes abundantly expressed GPR68, suggesting a role in reactive astrogliosis.

Two probesets for the DOC1 gene were significantly upregulated by Polimunol relative to Copaxone®. DOC1 is also known as CDK2AP1, or cyclin-dependent kinase 2 associated protein 1. DOC1 has been implicated as a locus for susceptibility to MS. (International Multiple Sclerosis Genetics Consortium (IMSGC), Genes Immun., 2010). One of the probesets downregulated by Polimunol relative to Copaxone was ADRB2, the gene encoding the beta-2 adrenergic receptor. Agonists for this receptor have been reported to affect antigen cross-presentation by dendritic cells (Hervé et al, J Immunol, 2013), alter cytokine secretion in human PBMC (Hilbert et al, Plos One, 2013), and change the proportions of myeloid cells in mouse brain under TNFα treatment (Laureys et al, J Neuroinflammation, 2014). In addition, signaling via this receptor in FOXP3+ regulatory T cells has been shown to enhance the suppressive function of these cells (Guereschi et al, Eur J Immunol, 2013). This functionality coupled with the observed expression levels of ADRB2 in FIG. 26 warrants further investigation.

These initial studies show clear and significant differences between Polimunol and Copaxone® in terms of impact on genomic profiling, including expression of genes with relevance for safety. Follow-on experiments are currently underway in both THP-1 cells and other model systems to further elucidate the differences identified to date. Substantial differences between Polimunol and Copaxone® were also identified using physicochemical methods and biological methods. Therefore, the gene expression differences described here, albeit subtle in terms of fold change, may represent chronic treatment effects that could have a clinically significant impact, and warrant further investigation in the interest of keeping MS patients safe.

### Example 10

### Differences in Gene Expression Induced by Polimunol Versus Branded GA in Mouse Splenocyte Gene Expression Studies

Immunological cells, particularly T cells, are critical to the antigenic mechanism of action of Copaxone®, thus post-treatment gene expression modulation needs to examine such relevant cell types, including lymphocytes.

As one approach to modeling these effects, a splenocyte system was utilized, in which mice were first immunized with either Copaxone® or Polimunol, and then sacrificed, having the splenocytes stimulated ex-vivo with Copaxone®, Polimunol, or medium. This model was used to simulate switching between medications compared with consistent use of one medicine or the other. A total of 157 samples were tested, and gene expression was measured using the Mouse Genome 430 2.0 Affymetrix chip.

Some similarities are observed between the genes modulated by Copaxone® in a mouse splenocyte model and in a human monocyte (THP-1) model: a prior study indicated that 1,378 genes were modulated by

Copaxone in both systems, while another 6,691 were modulated only in splenocytes and another 2,121 were modulated only in THP-1 cells (MSBoston Joint ACTRIMS-ECTRIMS 2014 Meeting, P282). These findings indicate that the majority of the impact of glatiramoids is cell type and tissue specific, including different interactions with lymphocytes versus antigen presenting cells, and thus several different immunologically relevant model systems are required to study the various implications of treatment with this NBCD.

### Copaxone®-Modulated Genes Mechanism of Action (MoA)

Analyzing splenocytes from mice immunized by Copaxone® and later activated with Copaxone®, 16,647 probesets were significantly modulated relative to medium (8,342 upregulated and 8,305 downregulated). The fact that over one third of the total probesets expressed in this tissue (i.e. 22,524 probesets called present on the chip) are significantly downregulated, and similarly over one third significantly upregulated, by Copaxone® treatment demonstrates the complexity of Copaxone's mechanism of action in this model system.

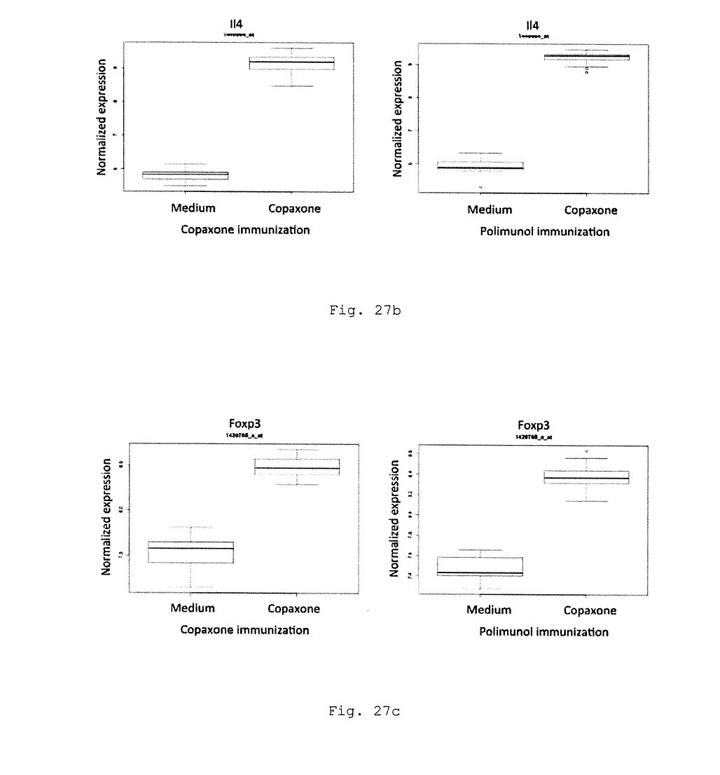

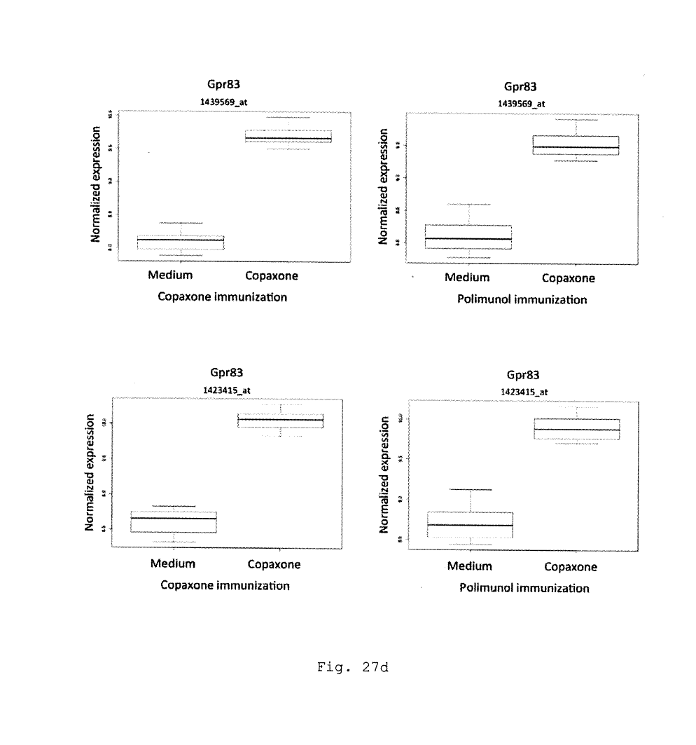

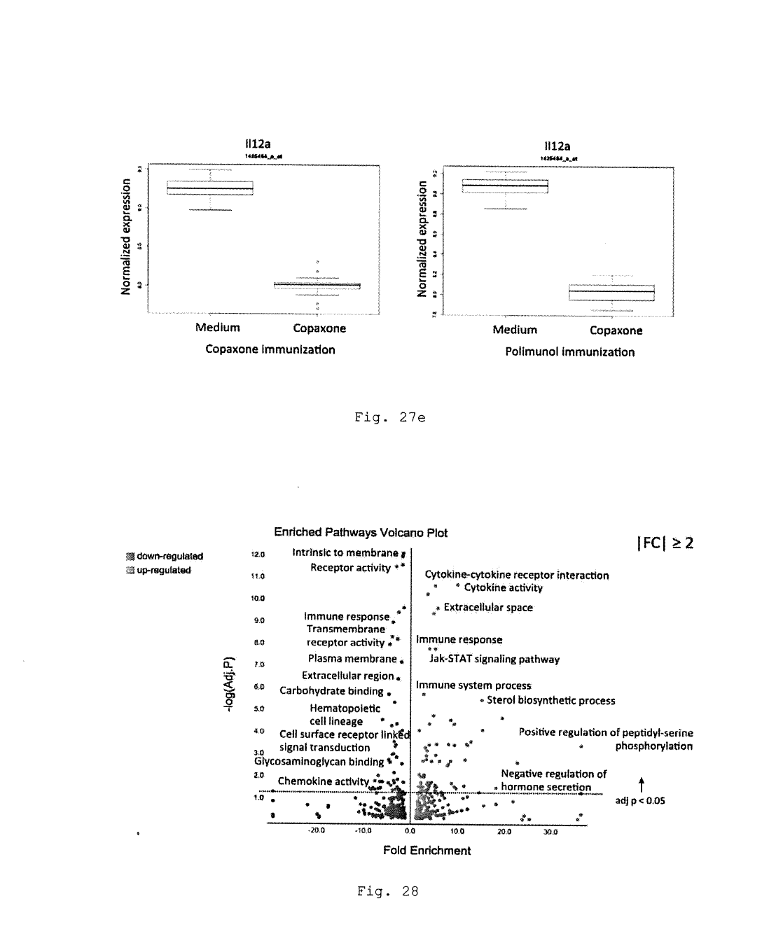

Copaxone® treatment upregulated key anti-inflammatory cytokines I110 and 114 (adj p<2.3e-24 and 5.1e-35, respectively; FIG. 27a-b) as well as markers of regulatory T cells, Foxp3 and Gpr83 (adj p<3.4e-23 for Foxp3; adj p<4.2e-33 and 9.0e-31 for the two Gpr83 probesets; FIG. 27c-d). Copaxone® downregulated pro-inflammatory cytokines, such as 1112a (adjusted p<8.3e-31; FIG. 27e). Consistently similar effects were observed for both immunization conditions (p values given above correspond to Copaxone® immunization, and are within the same order of magnitude for immunization by Polimunol).

### Copaxone®-Modulated Pathways

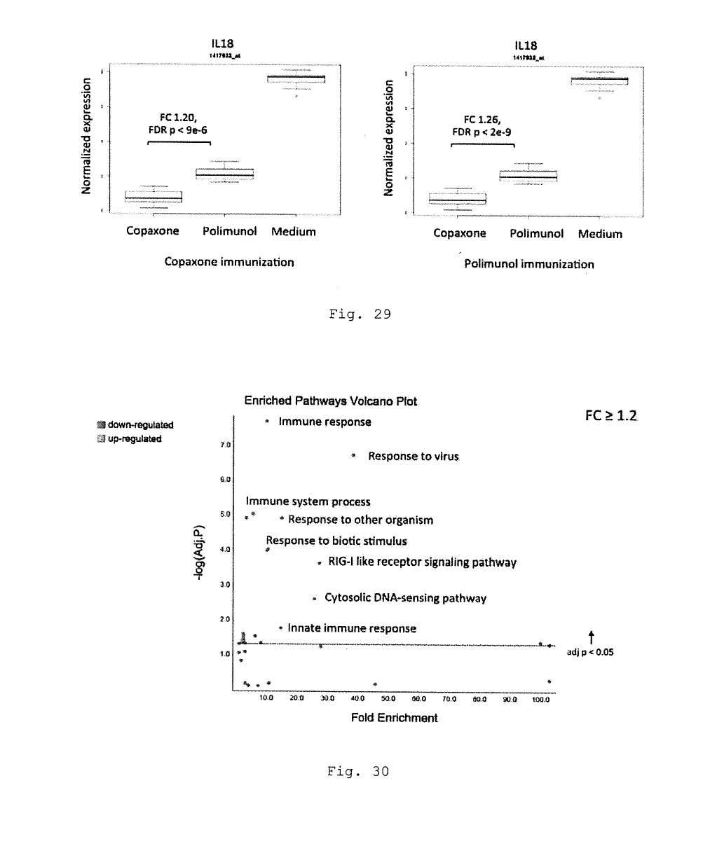

After imposing a conservative fold change filter of |FC|≧2, 411 probesets are upregulated by Copaxone® relative to medium (in Copaxone-immunized mice), and these probesets enrich for 76 pathways. These pathways include relevant aspects of Copaxone®'s mechanism of action such as the cytokine-cytokine receptor interaction pathway identified previously in the THP-1 study for Copaxone mechanism of action (as well as differences observed with Polimunol). Similarly, 485 downregulated probesets are detected, which enrich for 56 pathways. Both the upregulated and downregulated pathways are depicted in FIG. 28. The full list of pathways entiched among top probesets modulated by Copaxone® relative to medium is provided in Table 15.

TABLE 15
Pathways enriched among top probesets modulated by Copaxone ®
relative to medium in splenocytes from mice immunized with Copaxone ®
(Pathways enriched among top upregulated probesets listed first
followed by pathways enriched among top downregulated probesets).

| Categery | Term | Count | Pvalue | Fold Enrichment | Benjamini |
|---|---|---|---|---|---|
| GOTERM_MF_ALL | GO: 0005125-cytokine activity | 20 | 4.24E−14 | 9.93177388 | 2.11E−11 |
| KEGG_PATHWAY | mmu04060: Cytokine-cytokine receptor interaction | 29 | 1.86E−13 | 5.15544371 | 2.36E−11 |
| GOTERM_CC_ALL | GO: 0005576-extracellular region | 44 | 2.62E−13 | 3.56241762 | 5.65E−11 |
| GOTERM_CC_ALL | GO: 0005615-extracellular space | 26 | 2.29E−12 | 5.71896261 | 2.47E−10 |
| GOTERM_CC_ALL | GO: 0044421-extracellular region part | 29 | 5.96E−12 | 4.84008694 | 4.29E−10 |
| GOTERM_BP_ALL | GO: 0006955-immune response | 34 | 1.04E−11 | 3.99299275 | 1.66E−08 |
| KEGG_PATHWAY | mmu04630: lak-STAT signaling pathway | 22 | 2.63E−10 | 5.24077519 | 1.67E−08 |
| GOTERM_BP_ALL | GO: 0002376-immune system process | 42 | 2.48E−09 | 2.76506571 | 1.98E−06 |
| GOTERM_BP_ALL | GO: 0016126-sterol biosynthetic process | 10 | 6.57E−09 | 15.1694577 | 3.49E−06 |
| GOTERM_CC_ALL | GO: 0009897-external side of plasma membrane | 17 | 3.35E−07 | 4.86392974 | 1.81E−05 |
| GOTERM_BP_ALL | GO: 0008299-isoprenoid biosynthetic process | 8 | 5.51E−08 | 19.4169059 | 2.20E−05 |
| GOTERM_BP_ALL | GO: 0016125-sterol metabolic process | 12 | 8.93E−08 | 8.56628202 | 2.85E−05 |
| GOTERM_CC_ALL | GO: 0009986-cell surface | 19 | 9.95E−07 | 4.01671583 | 4.30E−05 |
| GOTERM_BP_ALL | GO: 0006694-steroid biosynthetic process | 11 | 1.64E−07 | 9.31334149 | 4.35E−05 |
| GOTERM_BP_ALL | GO: 0006695-cholesterol biosynthetic process | 8 | 3.93E−07 | 15.3291362 | 7.83E−05 |
| GOTERM_BP_ALL | GO: 0050896-response to stimulus | 65 | 3.63E−07 | 1.84733443 | 8.26E−05 |
| KEGG_PATHWAY | mmu00900: Terpenoid backbone biosynthesis | 7 | 6.27E−06 | 12.8270722 | 0.00026558 |
| KEGG_PATHWAY | mmu00100: Steroid biosynthesis | 7 | 1.06E−05 | 11.9108527 | 0.00033672 |
| GOTERM_BP_ALL | GO: 0008202-steroid metabolic process | 13 | 2.89E−06 | 5.56808331 | 0.00035349 |
| GOTERM_BP_ALL | GO: 0008203-cholesterol metabolic process | 10 | 2.70E−06 | 8.09037746 | 0.00035891 |
| GOTERM_BP_ALL | GO: 0008284-positive regulation of cell proliferation | 18 | 2.13E−06 | 3.99585716 | 0.00037779 |
| GOTERM_BP_ALL | GO: 0006720-isoprenoid metabolic process | 8 | 2.40E−06 | 12.1355662 | 0.00038277 |
| GOTERM_BP_ALL | GO: 0033138-positive regulation of peptidyl-serine phospherylation | 5 | 2.66E−06 | 36.4066986 | 0.00038457 |
| GOTERM_BP_ALL | GO: 0050730-regulation of peptidyl-tyrosine phosphorylation | 9 | 3.40E−06 | 9.36172249 | 0.00038657 |
| GOTERM_MF_ALL | GO: 0005102-receptor binding | 24 | 2.03E−06 | 3.13177103 | 0.00050318 |
| GOTERM_BP_ALL | GO: 0042325-regulation of phosphorylation | 19 | 6.54E−06 | 3.51130595 | 0.00069403 |
| GOTERM_BP_ALL | GO: 0001932-regulation of protein amino acid phosphorylation | 12 | 1.11E−05 | 5.39358497 | 0.00110231 |
| GOTERM_BP_ALL | GO: 0051174-regulation of phosphorus metabolic process | 19 | 1.22E−05 | 3.35789938 | 0.00114199 |
| GOTERM_BP_ALL | GO: 0019220-regulation of phosphate metabolic process | 19 | 1.22E−05 | 3.35789938 | 0.00114199 |
| GOTERM_BP_ALL | GO: 0008610-lipid biosynthetic process | 18 | 2.07E−05 | 3.3779411 | 0.00173185 |
| GOTERM_BP_ALL | GO: 0050731-positive regulation of peptidyl-tyresine phosphorylation | 7 | 2.01E−05 | 11.5839495 | 0.00177599 |
| GOTERM_BP_ALL | GO: 0042127-regulation of cell proliferation | 24 | 2.57E−05 | 2.68024775 | 0.00204526 |
| GOTERM_BP_ALL | GO: 0001934-positive regulation of protein amino acid | 8 | 2.96E−05 | 8.56628202 | 0.00208413 |
| | phosphorylation | | | | |
| GOTERM_BP_ALL | GO: 0010740-positive regulation of protein kinase cascade | 10 | 2.75E−05 | 6.17062688 | 0.00208529 |
| GOTERM_BP_ALL | GO: 0010627-regulation of protein kinase cascade | 13 | 2.92E−05 | 4.46497247 | 0.00210456 |
| GOTERM_BP_ALL | GO: 0033135-regulation of peptidyl-serine phosphorylation | 5 | 3.48E−05 | 22.7541866 | 0.00231024 |
| GOTERM_BP_ALL | GO: 0042327-positive regulation of phosphorylation | 8 | 4.38E−05 | 8.09037746 | 0.00279017 |
| GOTERM_BP_ALL | GO: 0045937-positive regulation of phosphate metabolic process | 8 | 5.28E−05 | 7.87171861 | 0.00323223 |
| GOTERM_BP_ALL | GO: 0010562-positive regulation of phosphorus metabolic process | 8 | 5.28E−05 | 7.87171861 | 0.00323223 |
| GOTERM_BP_ALL | GO: 0048522-positive regulation of cellular process | 41 | 0.00014045 | 1.83826926 | 0.00825294 |
| GOTERM_BP_ALL | GO: 0006952-defense response | 18 | 0.0001494 | 2.88687478 | 0.00846421 |
| GOTERM_BP_ALL | GO: 0006629-lipid metabolic process | 26 | 0.00019818 | 2.24306674 | 0.01046917 |
| GOTERM_BP_ALL | GO: 0006066-alcohol metabolic process | 18 | 0.00019444 | 2.82465765 | 0.01062493 |
| GOTERM_BP_ALL | GO: 0051251-positive regulation of lymphocyte activation | 10 | 0.00037281 | 4.43984129 | 0.01839122 |
| GOTERM_BP_ALL | GO: 0031399-regulation of protein modification process | 12 | 0.00036131 | 3.70237613 | 0.01839468 |
| GOTERM_BP_ALL | GO: 0008219-cell death | 24 | 0.00038581 | 2.24041222 | 0.01845504 |
| GOTERM_BP_ALL | GO: 0006915-apoptosis | 23 | 0.00041493 | 2.28161871 | 0.01925699 |
| GOTERM_BP_ALL | GO: 0016265-death | 24 | 0.00049622 | 2.20090873 | 0.02006939 |
| GOTERM_BP_ALL | GO: 0002696-positive regulation of leukocyte activation | 10 | 0.00044678 | 4.33413078 | 0.02013394 |
| GOTERM_BP_ALL | GO: 0012501-programmed cell death | 23 | 0.00046402 | 2.2631191 | 0.02032837 |
| GOTERM_BP_ALL | GO: 0002694-regulation of leukocyte activation | 12 | 0.0005166 | 3.55187303 | 0.0203686 |
| GOTERM_BP_ALL | GO: 0050867-positive regulation of cell activation | 10 | 0.00053242 | 4.23333704 | 0.02047943 |
| GOTERM_BP_ALL | GO: 0043410-positive regulation of MAPKKK cascade | 6 | 0.00049453 | 8.73760766 | 0.02052261 |
| GOTERM_BP_ALL | GO: 0009967-positive regulation of signal transduction | 11 | 0.00048296 | 3.88809402 | 0.02058357 |
| GOTERM_BP_ALL | GO: 0050865-regulation of cell activation | 12 | 0.00059269 | 3.49504306 | 0.02223545 |
| GOTERM_BP_ALL | GO: 0031401-positive regulation of protein modification process | 8 | 0.0006867 | 5.29551979 | 0.02512736 |
| GOTERM_BP_ALL | GO: 0010647-positive regulation of cell communication | 11 | 0.0007042 | 3.70808967 | 0.02518163 |
| GOTERM_BP_ALL | GO: 0051048-negative regulation of secretion | 5 | 0.00076246 | 11.3770933 | 0.02664025 |
| GOTERM_BP_ALL | GO: 0048568-embryonic organ development | 10 | 0.00080674 | 4.00073611 | 0.02756213 |
| GOTERM_BP_ALL | GO: 0010604-positive regulation of macromolecule metabolic process | 23 | 0.00085004 | 2.1637056 | 0.02841182 |
| GOTERM_BP_ALL | GO: 0048518-positive regulation of biological process | 42 | 0.0009007 | 1.66566595 | 0.0288702 |
| GOTERM_MF_ALL | GO: 0016705-oxidoreductase activity, acting on paired donors, | 9 | 0.00029543 | 5.1901528 | 0.02894292 |
| | with incorporation | | | | |
| GOTERM_BP_ALL | GO: 0044255-cellular lipid metabolic process | 20 | 0.00088925 | 2.32630662 | 0.02909335 |
| GOTERM_MF_ALL | GO: 0042379-chemokine receptor binding | 6 | 0.00035759 | 9.32723112 | 0.02919116 |
| GOTERM_MF_ALL | GO: 0004888-transmembrane receptor activity | 18 | 0.00042544 | 2.64847303 | 0.02976082 |
| GOTERM_MF_ALL | GO: 0019955-cytokine binding | 9 | 0.00018429 | 5.54809437 | 0.0300713 |
| GOTERM_BP_ALL | GO: 0046888-negative regulation of hormone secretion | 4 | 0.0010187 | 18.2033493 | 0.03195084 |
| GOTERM_MF_ALL | GO: 0008009-chemokine activity | 6 | 0.00028629 | 9.75119617 | 0.03495115 |
| GOTERM_BP_ALL | GO: 0042180-cellular ketone metabolic process | 20 | 0.00120964 | 2.26833013 | 0.0364004 |
| GOTERM_BP_ALL | GO: 0016053-organic acid biosynthetic process | 10 | 0.00118661 | 3.79236443 | 0.03640669 |
| GOTERM_BP_ALL | GO: 0046394-carboxylic acid biosynthetic process | 10 | 0.00118661 | 3.79236443 | 0.03640669 |
| GOTERM_BP_ALL | GO: 0051249-regulation of lymphocyte activation | 11 | 0.00131104 | 3.422852 | 0.0386639 |
| KEGG_PATHWAY | mmu05320: Autoimmune thyroid disease | 7 | 0.00172331 | 5.21099806 | 0.04286406 |
| GOTERM_BP_ALL | GO: 0009611-response to wounding | 14 | 0.00154486 | 2.77007489 | 0.04458422 |
| GOTERM_BP_ALL | GO: 0006954-inflammatory response | 11 | 0.00158927 | 3.3372807 | 0.04502272 |
| GOTERM_BP_ALL | GO: 0050864-regulation of B cell activation | 7 | 0.00163582 | 5.42227425 | 0.04550354 |
| GOTERM_CC_ALL | GO: 0044425-membrane part | 167 | 3.13E−15 | 1.63662801 | 7.90E−13 |
| GOTERM_CC_ALL | GO: 0031224-intrinsic to membrane | 140 | 8.73E−15 | 1.77744709 | 1.11E−12 |
| GOTERM_CC_ALL | GO: 0016021-integral to membrane | 135 | 3.09E−14 | 1.78531887 | 2.61E−12 |
| GOTERM_MF_ALL | GO: 0004872-receptor activity | 59 | 6.67E−15 | 3.07839222 | 3.10E−12 |
| GOTERM_CC_ALL | GO: 0016020-membrane | 172 | 3.65E−12 | 1.50486701 | 2.32E−10 |
| GOTERM_MF_ALL | GO: 0004871-signal transducer activity | 63 | 1.62E−12 | 2.59629647 | 3.77E−10 |
| GOTERM_MF_ALL | GO: 0060089-molecular transducer activity | 63 | 1.62E−12 | 2.59629647 | 3.77E−10 |
| GOTERM_BP_ALL | GO: 0006955-immune response | 40 | 6.54E−13 | 3.77617866 | 1.06E−09 |
| GOTERM_MF_ALL | GO: 0004888-transmembrane receptor activity | 33 | 3.17E−11 | 3.96796318 | 4.91E−09 |
| GOTERM_BP_ALL | GO: 0002376-immune system process | 53 | 7.56E−12 | 2.80481986 | 6.14E−09 |
| GOTERM_CC_ALL | GO: 0009986-cell surface | 27 | 2.11E−10 | 4.47916667 | 1.07E−08 |
| GOTERM_CC_ALL | GO: 0005886-plasma membrane | 83 | 1.60E−09 | 1.91980807 | 6.79E−08 |
| GOTERM_CC_ALL | GO: 0005576-extracellular region | 42 | 1.08E−08 | 2.66843972 | 3.93E−07 |
| GOTERM_MF_ALL | GO: 0030246-carbohydrate binding | 21 | 1.71E−08 | 4.64841968 | 1.99E−06 |
| GOTERM_CC_ALL | GO: 0044421-extracellular region part | 26 | 1.42E−07 | 3.40521443 | 4.51E−06 |
| KEGG_PATHWAY | mmu04640: Hematopoietic cell lineage | 14 | 2.50E−07 | 5.97693804 | 2.75E−05 |
| GOTERM_BP_ALL | GO: 0009605-response to external stimulus | 33 | 7.98E−08 | 2.90017325 | 4.32E−05 |
| GOTERM_CC_ALL | GO: 0009897-external side of plasma membrane | 18 | 1.84E−06 | 4.04135338 | 5.20E−05 |
| GOTERM_BP_ALL | GO: 0050896-response to stimulus | 77 | 2.55E−07 | 1.75912148 | 8.30E−05 |
| GOTERM_BP_ALL | GO: 0007166-cell surface receptor linked signal transduction | 43 | 2.53E−07 | 2.3390549 | 0.00010269 |
| GOTERM_BP_ALL | GO: 0009611-response to wounding | 22 | 9.96E−07 | 3.49912207 | 0.00026984 |
| GOTERM_BP_ALL | GO: 0002682-regulation of immune system process | 25 | 1.60E−06 | 3.07409502 | 0.00037039 |
| GOTERM_CC_ALL | GO: 0005615-extracellular space | 19 | 1.75E−05 | 3.279544 | 0.00044365 |
| GOTERM_BP_ALL | GO: 0002684-positive regulation of immune system process | 19 | 4.74E−06 | 3.58736973 | 0.00096165 |
| GOTERM_BP_ALL | GO: 0048584-positive regulation of response to stimulus | 17 | 5.63E−06 | 3.91741369 | 0.0010166 |
| GOTERM_BP_ALL | GO: 0006952-defense response | 23 | 8.45E−06 | 2.96521518 | 0.00137227 |
| GOTERM_MF_ALL | GO: 0005529-sugar binding | 13 | 1.92E−05 | 4.6322231 | 0.00178054 |
| GOTERM_BP_ALL | GO: 0050778-positive regulation of immune response | 14 | 1.56E−05 | 4.3586743 | 0.00230833 |
| GOTERM_CC_ALL | GO: 0044459-plasma membrane part | 46 | 0.00011625 | 1.79088802 | 0.00268093 |
| GOTERM_BP_ALL | GO: 0050776-regulation of immune response | 16 | 5.03E−05 | 3.46849003 | 0.00679349 |
| GOTERM_MF_ALL | GO: 0005539-glycosaminoglycan binding | 8 | 0.00012123 | 6.87497365 | 0.00935188 |
| GOTERM_BP_ALL | GO: 0007155-cell adhesion | 20 | 0.00010998 | 2.74792344 | 0.0136546 |
| GOTERM_BP_ALL | GO: 0022610-biological adhesion | 20 | 0.00010998 | 2.74792344 | 0.0136546 |
| GOTERM_BP_ALL | GO: 0007626-locomotory behavior | 14 | 0.0001342 | 3.56274248 | 0.01545668 |
| GOTERM_MF_ALL | GO: 0008201-heparin binding | 7 | 0.00024088 | 7.57520244 | 0.01587561 |
| GOTERM_MF_ALL | GO: 0001871-pattern binding | 8 | 0.00029954 | 5.99356677 | 0.01726258 |
| GOTERM_MF_ALL | GO: 0030247-polysaccharide binding | 8 | 0.00029954 | 5.99356677 | 0.01726258 |
| GOTERM_BP_ALL | GO: 0006909-phagocytosis | 8 | 0.00017473 | 6.5034188 | 0.01759075 |
| GOTERM_BP_ALL | GO: 0032501-multicellular organismal process | 77 | 0.00017032 | 1.47960251 | 0.01828376 |
| GOTERM_BP_ALL | GO: 0006954-inflammatory response | 14 | 0.00020677 | 3.41429487 | 0.01957233 |
| GOTERM_BP_ALL | GO: 0042330-taxis | 10 | 0.00022296 | 4.72022333 | 0.01992884 |
| GOTERM_BP_ALL | GO: 0006935-chemotaxis | 10 | 0.00022296 | 4.72022333 | 0.01992884 |
| GOTERM_CC_ALL | GO: 0005578-proteinaceous extracellular matrix | 9 | 0.00121761 | 4.19921875 | 0.0254587 |
| GOTERM_BP_ALL | GO: 0007186-G-protein coupled receptor protein signaling pathway | 17 | 0.00035255 | 2.79500864 | 0.02970748 |
| GOTERM_MF_ALL | GO: 0001664-G-protein-coupled receptor binding | 7 | 0.00063894 | 6.39157706 | 0.03248313 |
| GOTERM_MF_ALL | GO: 0008009-chemokine activity | 6 | 0.00072678 | 7.96871945 | 0.03324267 |
| GOTERM_BP_ALL | GO: 0008037-cell recognition | 6 | 0.00044777 | 8.77961539 | 0.03406274 |
| GOTERM_BP_ALL | GO: 0050900-leukocyte migration | 7 | 0.00043774 | 6.82858974 | 0.03494842 |
| GOTERM_MF_ALL | GO: 0042379-chemokine receptor binding | 6 | 0.00090307 | 7.62225339 | 0.03747238 |
| GOTERM_MF_ALL | GO: 0004930-G-protein coupled receptor activity | 12 | 0.0010892 | 3.24651533 | 0.03823098 |
| GOTERM_BP_ALL | GO: 0048583-regulation of response to stimulus | 19 | 0.00053982 | 2.50469508 | 0.03909905 |
| GOTERM_BP_ALL | GO: 0060326-cell chemotaxis | 6 | 0.00057152 | 8.36153846 | 0.03958565 |
| GOTERM_BP_ALL | GO: 0030595-leukocyte chemotaxis | 6 | 0.00057152 | 8.36153846 | 0.03958565 |
| GOTERM_MF_ALL | GO: 0005102-receptor binding | 21 | 0.00105337 | 2.23938466 | 0.04001704 |
| GOTERM_CC_ALL | GO: 0031012-extracellular matrix | 9 | 0.00218595 | 3.83928571 | 0.04185565 |
| GOTERM_BP_ALL | GO: 0007165-signal transduction | 51 | 0.00074467 | 1.58780278 | 0.04918842 |

### Copaxone® MoA Discussion

More than two thirds of the probesets expressed in the spleen were significantly modulated by Copaxone® treatment, and the top modulated probesets in either direction were enriched for many relevant pathways, including immune response and cytokine-cytokine receptor interaction pathways. Key anti-inflammatory cytokines, such as 1110 and 114, and regulatory T cell markers, such as Foxp3 and Gpr83, are upregulated by Copaxone®, while pro-inflammatory cytokines, such as 1112a, is downregulated by Copaxone® treatment in this model system, consistent with induction of a Th1 to Th2 shift. Thus, these studies help to illustrate the complexity of Copaxone's mechanism of action (impacting thousands of genes in over 100 different pathways), and provide validation of the experimental system

### Polimunol and Copaxone Comparison: Differentially Expressed Genes, Under Copaxone Immunization

When splenocytes from Copaxone-immunized mice were activated with Polimunol and compared to activation by Copaxone® (as reported in the prior section), controlling for mannitol control, 223 probesets were found to be differentially expressed (208 were upregulated, 15 were downregulated). The top 25 upregulated probesets by Polimunol versus Copaxone are reported in Table 16, and include many interferon-induced genes as will be further discussed in the pathway enrichment section below. The 15 downregulated probesets by Polimunol versus Copaxone® are reported in Table 17.

### Polimunol and Copaxone® Comparison: Differentially Expressed Genes, Under Polimunol Immunization

When splenocytes from Polimunol-immunized mice were activated with Copaxone® versus Polimunol, 431 probesets were found to be differentially expressed when both were corrected for medium (301 were upregulated, 130 were downregulated). The top 25 upregulated probesets by Polimunol versus Copaxone® are reported in Table 18 and consistently with the reciprocal study design (prior paragraph) include many interferon induced genes, and the top 25 downregulated probesets by Polimunol versus Copaxone® are reported in Table 19.

TABLE 16
Top 25 probesets upregulated by Polimunol versus Copaxone ®
In splenocytes from Copaxone ®-immunized mice

| Probeset | Gene | raw.FC | P.Value | adj.P.Val. |
|---|---|---|---|---|
| 1421551_s_at | Ifi202b | 1.48 | 6.69E−15 | 3.02E−10 |
| 1451905_a_at | Mx1 | 1.75 | 2.21E−14 | 4.98E−10 |
| 1421998_at | Tor3a | 1.29 | 9.33E−14 | 1.40E−09 |
| 1457666_s_at | Ifi202b /// | 1.49 | 1.25E−13 | 1.41E−09 |
| | LOC100044068 | | | |
| 1424339_at | Oasl1 | 1.46 | 2.05E−13 | 1.69E−09 |
| 1418293_at | Ifit2 | 1.38 | 2.24E−13 | 1.69E−09 |
| 1423555_a_at | Ifi44 | 1.62 | 2.85E−13 | 1.84E−09 |
| 1425917_at | Ifi44l | 1.63 | 4.06E−12 | 2.29E−08 |
| 1421596_s_at | Ifi44l | 1.63 | 1.40E−11 | 7.03E−08 |
| 1456164_at | AW011738 | 1.20 | 2.32E−11 | 9.03E−08 |
| 1450454_at | Tor3a | 1.25 | 2.40E−11 | 9.03E−08 |
| 1449455_at | Hck | 1.16 | 4.06E−11 | 1.33E−07 |
| 1448775_at | Gm16340 /// Ifi203 /// | 1.21 | 4.16E−11 | 1.33E−07 |
| | LOC100862473 | | | |
| 1438868_at | Phf11d | 1.25 | 4.42E−11 | 1.33E−07 |
| 1428660_s_at | Tor3a | 1.25 | 5.25E−11 | 1.41E−07 |
| 1434401_at | Zcchc2 | 1.16 | 5.31E−11 | 1.41E−07 |
| 1418191_at | Usp18 | 1.39 | 1.10E−10 | 2.71E−07 |
| 1450027_at | Sdc3 | 1.25 | 1.14E−10 | 2.71E−07 |
| 1435331_at | Pyhin1 | 1.19 | 2.12E−10 | 4.79E−07 |
| 1436120_at | Setdb2 | 1.16 | 2.43E−10 | 5.21E−07 |
| 1451330_a_at | Inpp5b | 1.12 | 3.00E−10 | 6.14E−07 |
| 1424921_at | Bst2 | 1.23 | 3.99E−10 | 7.83E−07 |
| 1419603_at | Ifi204 | 1.44 | 5.16E−10 | 9.70E−07 |
| 1438037_at | Herc6 | 1.27 | 6.06E−10 | 1.09E−06 |
| 1419026_at | Daxx | 1.21 | 6.74E−10 | 1.7E−06 |

TABLE 17
15 probesets downregulated by Polimunol versus Copaxone ® In
splenocytes from Copaxone ®-immunized mice

| Probeset | Gene | raw.FC | P.Value | adj.P.Val. |
|---|---|---|---|---|
| 1454757_s_at | Ifi27l1 | −1.15 | 1.92E−08 | 1.57E−05 |
| 1415837_at | Klk1 | −1.41 | 4.55E−08 | 3.16E−05 |
| 1452956_a_at | Ifi27l1 | −1.14 | 1.80E−07 | 9.56E−05 |
| 1448107_x_at | Klk1 | −1.34 | 2.21E−07 | 0.000114521 |
| 1439771_s_at | D13ERTD608E | −1.25 | 2.71E−07 | 0.00013591 |
| 1415823_at | Scd2 | −1.35 | 3.68E−06 | 0.001275671 |
| 1442544_at | Ighm | −1.17 | 5.89E−06 | 0.001916738 |
| 1415824_at | Scd2 | −1.31 | 2.36E−05 | 0.006376176 |
| 1415822_at | Scd2 | −1.28 | 3.43E−05 | 0.008489827 |
| 1429100_at | Ccdc71l | −1.08 | 3.68E−05 | 0.008965532 |
| 1427756_x_at | Ighm | −1.17 | 4.61E−05 | 0.010874062 |
| 1449325_at | Fads2 | −1.16 | 0.000104782 | 0.022291363 |
| 1416188_at | Gm2a | −1.08 | 0.000109828 | 0.023255206 |
| 1424567_at | Tspan2 | −1.13 | 0.00016899 | 0.033873925 |
| 1436131_at | Siglech | −1.12 | 0.000216398 | 0.042416254 |

TABLE 18
Top 25 probesets upregulated by Polimunol versus Copaxone ® in
splenocytes from Polimunol-immunized mice

| Probeset | Gene | raw.FC | P.Value | adj.P.Val. |
|---|---|---|---|---|
| 1434401_at | Zcchc2 | 1.17 | 6.74E−14 | 1.96E−09 |
| 1417932_at | Il18 | 1.26 | 8.67E−14 | 1.96E−09 |
| 1457035_at | AI607873 | 1.43 | 1.41E−12 | 2.10E−08 |
| 1421551_s_at | Ifi202b | 1.43 | 1.86E−12 | 2.10E−08 |
| 1419599_s_at | Ms4a6d | 1.38 | 5.58E−12 | 5.04E−08 |
| 1456164_at | AW011738 | 1.21 | 1.21E−11 | 9.11E−08 |
| 1457666_s_at | Ifi202b /// | 1.44 | 5.60E−11 | 2.73E−07 |
| | LOC100044068 | | | |
| 1449591_at | Casp4 | 1.11 | 6.06E−11 | 2.73E−07 |
| 1440865_at | Ifitm6 | 1.23 | 7.78E−11 | 3.19E−07 |
| 1438004_at | Papd7 | 1.19 | 9.92E−11 | 3.44E−07 |
| 1426721_s_at | Tiparp | 1.12 | 1.80E−10 | 5.81E−07 |
| 1426716_at | Tdrd7 | 1.21 | 2.85E−10 | 8.56E−07 |
| 1419598_at | Ms4a6d | 1.33 | 7.99E−10 | 2.17E−06 |
| 1420796_at | Ahrr | 1.47 | 1.38E−09 | 3.45E−06 |
| 1436058_at | Rsad2 | 1.52 | 2.07E−09 | 4.67E−06 |
| 1421008_at | Rsad2 | 1.40 | 2.22E−09 | 4.77E−06 |
| 1421009_at | Rsad2 | 1.53 | 2.56E−09 | 5.07E−06 |
| 1418293_at | Ifit2 | 1.42 | 2.58E−09 | 5.07E−06 |
| 1427102_at | Slfn4 | 1.42 | 2.86E−09 | 5.38E−06 |
| 1453939_x_at | Gm9706 | 1.28 | 3.33E−09 | 6.02E−06 |
| 1427747_a_at | Lcn2 | 1.21 | 3.54E−09 | 6.14E−06 |
| 1447851_x_at | Atp10a | 1.16 | 4.58E−09 | 7.37E−06 |
| 1450322_s_at | Slfh3 /// Slfn4 | 1.38 | 5.25E−09 | 8.17E−06 |
| 1438148_at | Cxcl3 | 1.68 | 6.34E−09 | 9.54E−06 |
| 1451905_a_at | Mx1 | 1.79 | 1.09E−08 | 1.54E−05 |

TABLE 19
Top 25 probesets downregulated by Polimunol versus Copaxone ® in
splenocytes from Polimunol-immunizad mice

| Probeset | Gene | raw.FC | P.Value | adj.P.Val. |
|---|---|---|---|---|
| 1436839_at | Ccdc71l | −1.14 | 3.56E−11 | 2.11E−07 |
| 1451461_a_at | Aldoc | −1.60 | 3.75E−11 | 2.11E−07 |
| 1454757_s_at | Ifi27l1 | −1.15 | 9.61E−11 | 3.44E−07 |
| 1442544_at | Ighm | −1.29 | 8.19E−10 | 2.17E−06 |
| 1452956_a_at | Ifi27l1 | −1.13 | 1.68E−09 | 3.99E−06 |
| 1417679_at | Gfi1 | −1.23 | 4.10E−09 | 6.85E−06 |
| 1417864_at | Pgk1 | −1.19 | 8.66E−09 | 1.26E−05 |
| 1429100_at | Ccdc71l | −1.12 | 1.45E−08 | 1.99E−05 |
| 1435086_s_at | Klhdc2 | −1.13 | 4.23E−08 | 4.33E−05 |
| 1451385_at | Fam162a | −1.17 | 1.15E−07 | 0.000102062 |
| 1417034_at | Trappc6a | −1.18 | 1.33E−07 | 0.000111102 |
| 1435659_a_at | Tpi1 | −1.37 | 1.69E−07 | 0.000128996 |
| 1415995_at | Casp6 | −1.13 | 2.32E−07 | 0.000164607 |
| 1450081_x_at | Gpi1 | −1.13 | 3.06E−07 | 0.000206058 |
| 1434814_x_at | Gpi1 | −1.11 | 4.41E−07 | 0.000287936 |
| 1418709_at | Cox7a1 | −1.31 | 4.64E−07 | 0.000298801 |
| 1417772_at | Grhpr | −1.23 | 4.84E−07 | 0.000307264 |
| 1439435_x_at | Pgk1 | −1.16 | 5.64E−07 | 0.000334169 |
| 1438640_x_at | Pgk1 | −1.15 | 7.54E−07 | 0.00040969 |
| 1417308_at | Pkm | −1.14 | 1.00E−06 | 0.000518315 |
| 1415918_a_at | Tpi1 | −1.31 | 1.08E−06 | 0.00055306 |
| 1424714_at | Aldoc | −1.32 | 1.23E−06 | 0.000599143 |
| 1418649_at | Egln3 | −1.27 | 1.29E−06 | 0.000610123 |
| 1452927_x_at | Tpi1 | −1.29 | 1.34E−06 | 0.000631391 |
| 1457587_at | Kcnq5 | −1.14 | 1.86E−06 | 0.000789811 |

It is important to note that for either immunization (Copaxone® or Polimunol), I118 and its receptor I118r1 are downregulated (and inhibitor I118 bp is upregulated) by both of the activation treatments, Copaxone® and Polimunol. I118 is downregulated significantly less by Polimunol than by Copaxone® (differential expression FDR p<9e-6 (FC 1.20) with Copaxone® immunization and FDR p<2e-9 (FC 1.26) for Polimunol immunization). Downregulation of both I118 and I118r1 expression upon Copaxone® treatment was also reported in an earlier splenocyte study of similar design (Bakshi et al, 2013). Boxplots of I118 expression are shown in FIG. 29.

The probesets significantly entiched among top probesets modulated by Copaxone® relative to medium is provided in Table 20.

TABLE 20
Top probesets significantly differentially expressed between Polimunol and Copaxone ® treatment
(corrected for medium control) in splenocytes from Copaxone ®-immunized mice.

| Probeset | Gene | raw. FC | AveExpr | t | P. Value | adj. P. Val | B |
|---|---|---|---|---|---|---|---|
| 1421551_s_a | Ifi202b | 1.47550912 | 9.16666894 | 10.5600946 | 6.69E−15 | 3.02E−10 | 22.5317107 |
| 1451905_a_a | Mx1 | 1.75432592 | 8.47709648 | 10.2249021 | 2.21E−14 | 4.98E−10 | 21.4748998 |
| 1421998_at | Tor3a | 1.2928856 | 9.17160227 | 9.82507613 | 9.33E−14 | 1.40E−09 | 20.1932231 |
| 1457666_s_a | Ifi202b /// L [symbol] | 1.49450011 | 8.60997589 | 9.74469879 | 1.25E−13 | 1.41E−09 | 19.9328881 |
| 1424339_at | Oasl1 | 1.46319835 | 9.8248556 | 9.60829298 | 2.05E−13 | 1.69E−09 | 19.489091 |
| 1418293_at | Ifit2 | 1.38359824 | 10.906172 | 9.5837092 | 2.24E−13 | 1.69E−09 | 19.4088447 |
| 1423555_a_a | Ifi44 | 1.62258196 | 9.94187992 | 9.51838876 | 2.85E−13 | 1.84E−09 | 19.1952407 |
| 1425917_at | Ifi44l | 1.6309207 | 8.85125178 | 8.79871072 | 4.06E−12 | 2.29E−08 | 16.8068255 |
| 1421596_s_a | Ifi44l | 1.62779642 | 8.55050842 | 8.4667453 | 1.40E−11 | 7.03E−08 | 15.6855236 |
| 1456164_at | AW011738 | 1.19734234 | 6.98570845 | 8.33256611 | 2.32E−11 | 9.03E−08 | 15.2292395 |
| 1450454_at | Tor3a | 1.25024933 | 8.74969803 | 8.32329605 | 2.40E−11 | 9.03E−08 | 15.1976556 |
| 1449455_at | Hck | 1.1582816 | 10.9515022 | 8.18380999 | 4.06E−11 | 1.33E−07 | 14.7215139 |
| 1448775_at | Gm16340 // [symbol] | 1.20618615 | 11.6014591 | 8.1770987 | 4.16E−11 | 1.33E−07 | 14.6985633 |
| 1438868_at | Phf11d | 1.2483759 | 10.329053 | 8.16092351 | 4.42E−11 | 1.33E−07 | 14.6432337 |
| 1428660_s_a | Tor3a | 1.248966 | 10.5033156 | 8.11568562 | 5.25E−11 | 1.41E−07 | 14.488379 |
| 1434401_at | Zcchc2 | 1.15603797 | 10.8249905 | 8.1125305 | 5.31E−11 | 1.41E−07 | 14.4775726 |
| 1418191_at | Usp18 | 1.38861948 | 11.5218281 | 7.91969756 | 1.10E−10 | 2.71E−07 | 13.8156858 |
| 1450027_at | Sdc3 | 1.25031078 | 10.4588938 | 7.9099888 | 1.14E−10 | 2.71E−07 | 13.7822905 |
| 1435331_at | Pyhin1 | 1.19292098 | 12.437413 | 7.74536994 | 2.12E−10 | 4.79E−07 | 13.2151145 |
| 1436120_at | Setdb2 | 1.15959289 | 10.4269299 | 7.71019806 | 2.43E−10 | 5.21E−07 | 13.0937189 |
| 1451330_a_a | Inpp5b | 1.12122817 | 10.7773832 | 7.65452165 | 3.00E−10 | 6.14E−07 | 12.90141 |
| 1424921_at | Bst2 | 1.22915197 | 11.6875461 | 7.57865485 | 3.99E−10 | 7.83E−07 | 12.6390995 |
| 1419603_at | Ifi204 | 1.43740773 | 7.15143963 | 7.51082104 | 5.16E−10 | 9.70E−07 | 12.4043263 |
| 1438037_at | Herc6 | 1.27135334 | 10.4464057 | 7.46845772 | 6.06E−10 | 1.09E−06 | 12.2576026 |
| 1419026_at | Daxx | 1.20586588 | 12.032791 | 7.44071108 | 6.74E−10 | 1.17E−06 | 12.1614627 |
| 1451567_a_a | Gm16340 // [symbol] | 1.14843448 | 12.2623403 | 7.42376782 | 7.18E−10 | 1.20E−06 | 12.1027407 |
| 1426716_at | Tdrd7 | 1.17546145 | 9.81553185 | 7.38758714 | 8.24E−10 | 1.33E−06 | 11.9773091 |
| 1426774_at | Parp12 | 1.23323427 | 10.568185 | 7.32082974 | 1.06E−09 | 1.60E−06 | 11.7457533 |
| 1426276_at | Ifih1 | 1.25213306 | 10.3259786 | 7.3202472 | 1.06E−09 | 1.60E−06 | 11.7437321 |
| 1435665_at | Trim30d | 1.25197418 | 9.71194292 | 7.26872528 | 1.29E−09 | 1.88E−06 | 11.5649255 |
| 1431591_s_a | Gm9706 /// [symbol] | 1.32351255 | 11.382458 | 7.17864536 | 1.82E−09 | 2.56E−06 | 11.2521346 |
| 1435330_at | Pyhin1 | 1.20956064 | 9.18347439 | 7.12657207 | 2.22E−09 | 3.03E−06 | 11.0712364 |
| 1438716_at | Trim30d | 1.23305159 | 7.93829998 | 7.11825503 | 2.29E−09 | 3.03E−06 | 11.0423392 |
| 1421008_at | Rsad2 | 1.40087837 | 7.80703835 | 7.0280384 | 3.22E−09 | 4.15E−06 | 10.7288224 |
| 1421571_a_a | Ly6c1 /// Ly6 | 1.12684658 | 13.613752 | 6.99594752 | 3.64E−09 | 4.56E−06 | 10.6172805 |
| 1435792_at | Csprs /// Gm | 1.187456 | 9.75936346 | 6.97563188 | 3.93E−09 | 4.76E−06 | 10.5466635 |
| 1423754_at | Ifitm3 | 1.19002363 | 12.4964655 | 6.96462013 | 4.10E−09 | 4.76E−06 | 10.5083857 |
| 1457035_at | Al607873 | 1.33928347 | 5.46136565 | 6.96314834 | 4.12E−09 | 4.76E−06 | 10.5032697 |
| 1453939_x_a | Gm9706 | 1.28772667 | 8.70376747 | 6.95300697 | 4.28E−09 | 4.83E−06 | 10.468017 |
| 1420671_x_a | Ms4a4c | 1.11351765 | 13.1959702 | 6.9122537 | 5.00E−09 | 5.50E−06 | 10.326351 |
| 1426721_s_a | Tiparp | 1.12038515 | 10.8883102 | 6.87404388 | 5.77E−09 | 6.20E−06 | 10.1935268 |
| 1427059_at | Tmem184b | 1.19049436 | 9.2053589 | 6.80169576 | 7.60E−09 | 7.97E−06 | 9.94204892 |
| 1453196_a_a | Oasl2 | 1.34151303 | 10.6174821 | 6.77476842 | 8.41E−09 | 8.62E−06 | 9.84846205 |
| 1452231_x_a | Mndal | 1.20215669 | 12.1489677 | 6.75397715 | 9.10E−09 | 9.12E−06 | 9.77620711 |
| 1417932_at | Il18 | 1.2024199 | 8.58526778 | 6.73898284 | 9.63E−09 | 9.44E−06 | 9.72410174 |
| 1420796_at | Ahrr | 1.35482305 | 6.39700067 | 6.68881415 | 1.16E−08 | 1.12E−05 | 9.54979119 |
| 1426906_at | Mndal | 1.22985583 | 12.0834069 | 6.68154198 | 1.20E−08 | 1.12E−05 | 9.52452794 |
| 1417244_a_a | Irf7 | 1.29690765 | 11.8707637 | 6.66482592 | 1.28E−08 | 1.17E−05 | 9.46646104 |
| 1456494_a_a | Trim30a /// [symbol] | 1.24228874 | 10.5953269 | 6.66073405 | 1.29E−08 | 1.17E−05 | 9.45224793 |
| 1445897_s_a | Ifi35 | 1.09502194 | 13.2112571 | 6.64901185 | 1.35E−08 | 1.20E−05 | 9.41153295 |
| 1418115_s_a | Tor1aip2 | 1.09066644 | 11.3826254 | 6.63787659 | 1.41E−08 | 1.22E−05 | 9.37285954 |
| 1426670_at | Agrn | 1.16650332 | 8.38464727 | 6.61334752 | 1.55E−08 | 1.32E−05 | 9.28767938 |
| 1450783_at | Ifit1 | 1.31370421 | 11.6154892 | 6.57611523 | 1.78E−08 | 1.49E−05 | 9.15841659 |
| 1436058_at | Rsad2 | 1.44054736 | 10.0892795 | 6.52152657 | 2.19E−08 | 1.76E−05 | 8.96897228 |
| 1460603_at | Samd9l | 1.09852241 | 13.0008563 | 6.48452217 | 2.52E−08 | 1.99E−05 | 8.84061078 |
| 1450484_a_a | Cmpk2 | 1.4069656 | 9.18452824 | 6.47037709 | 2.66E−08 | 2.06E−05 | 8.79155778 |
| 1452349_x_a | Ifi205 /// Mn [symbol] | 1.20045125 | 7.72773058 | 6.46703491 | 2.69E−08 | 2.06E−05 | 8.77996877 |
| 1435529_at | Gm14446 | 1.28471375 | 11.9844626 | 6.46256796 | 2.74E−08 | 2.06E−05 | 8.76448031 |
| 1455581_x_a | Gm20559 | 1.13805761 | 11.6732978 | 6.44922028 | 2.88E−08 | 2.09E−05 | 8.71820419 |
| 1451860_a_a | Trim30a | 1.18244967 | 12.1537452 | 6.36625749 | 3.94E−08 | 2.82E−05 | 8.43075121 |
| 1436562_at | Ddx58 | 1.14667035 | 11.473735 | 6.36165042 | 4.00E−08 | 2.82E−05 | 8.41479807 |
| 1448380_at | Lgals3bp | 1.16298116 | 11.2244145 | 6.30367575 | 4.98E−08 | 3.40E−05 | 8.21414149 |
| 1449025_at | Ifit3 | 1.3414478 | 11.8181697 | 6.30015076 | 5.05E−08 | 3.40E−05 | 8.20194708 |
| 1452087_at | Epsti1 | 1.11549904 | 11.2912312 | 6.24845347 | 6.13E−08 | 4.01E−05 | 8.02318829 |
| 1421009_at | Rsad2 | 1.51117184 | 7.74253963 | 6.2035856 | 7.26E−08 | 4.64E−05 | 7.86817911 |
| 1443698_at | Xaf1 | 1.18282113 | 11.9302802 | 6.20178329 | 7.30E−08 | 4.64E−05 | 7.86195528 |
| 1442130_at | Hsh2d | 1.1709592 | 10.2697537 | 6.16886582 | 8.27E−08 | 5.18E−05 | 7.7483219 |
| 1459151_x_a | Ifi35 | 1.10594094 | 11.6543585 | 6.13135928 | 9.50E−08 | 5.87E−05 | 7.62066485 |
| 1436172_at | Gm20559 | 1.1493816 | 11.5396111 | 6.07854249 | 1.16E−07 | 7.05E−05 | 7.43692632 |
| 1448575_at | Il7r | 1.09825437 | 11.0940999 | 6.07547051 | 1.17E−07 | 7.05E−05 | 7.4263466 |
| 1452160_at | Tiparp | 1.11979242 | 9.76721315 | 6.06904538 | 1.20E−07 | 7.13E−05 | 7.40422136 |
| 1451050_at | Nt5c3 | 1.16150561 | 10.6183063 | 6.05166841 | 1.28E−07 | 7.51E−05 | 7.34439991 |
| 1431095_a_a | Herc6 | 1.19901119 | 10.4733187 | 6.04385458 | 1.32E−07 | 7.63E−05 | 7.31750846 |
| 1423681_at | Ftsjd2 /// Rn [symbol] | 1.09083446 | 10.9815969 | 6.03884145 | 1.35E−07 | 7.68E−05 | 7.3002584 |
| 1449143_at | Rtp4 | 1.24304662 | 7.42935297 | 6.02268936 | 1.43E−07 | 8.05E−05 | 7.24469409 |
| 1437636_at | Pydc4 | 1.33382761 | 8.19945237 | 6.01217359 | 1.49E−07 | 8.27E−05 | 7.20853136 |
| 1450495_a_a | Klrk1 | 1.14444626 | 9.11777277 | 5.99801423 | 1.57E−07 | 8.59E−05 | 7.15985406 |
| 1450276_a_a | Scin | 1.26600893 | 8.7352575 | 5.99419456 | 1.59E−07 | 8.59E−05 | 7.14672584 |
| 1427091_at | Znfx1 | 1.16622599 | 9.5503153 | 5.99232677 | 1.60E−07 | 8.59E−05 | 7.1403067 |
| 1432026_a_a | Herc6 | 1.2332518 | 9.6340896 | 5.91285077 | 2.15E−07 | 0.00011289 | 6.86746993 |
| 1451777_at | Ddx60 | 1.2572541 | 9.17655647 | 5.89999373 | 2.26E−07 | 0.00011573 | 6.82339016 |
| 1451655_at | Slfn8 | 1.16818125 | 11.8030813 | 5.85259478 | 2.69E−07 | 0.00013591 | 6.6610318 |
| 1423071_x_a | 6720475J19 [symbol] | 1.12577119 | 8.9545691 | 5.84322799 | 2.79E−07 | 0.00013788 | 6.62897522 |
| 1450241_a_a | Evi2a | 1.11124373 | 11.3075457 | 5.84097845 | 2.81E−07 | 0.00013788 | 6.62127789 |
| 1421217_a_a | Lgals9 | 1.11289495 | 12.0356748 | 5.83616937 | 2.86E−07 | 0.00013886 | 6.60482433 |
| 1424775_at | Oas1a | 1.26381284 | 10.9682776 | 5.82921661 | 2.94E−07 | 0.00014098 | 6.58104095 |
| 1447851_x_a | Atp10a | 1.13412363 | 9.35777039 | 5.81806601 | 3.06E−07 | 0.0001454 | 6.54290906 |
| 1451426_at | Dhx58 | 1.27494236 | 10.5332943 | 5.79964551 | 3.28E−07 | 0.00015407 | 6.47994653 |
| 1452013_at | Atp10a | 1.16582075 | 7.25191599 | 5.74981475 | 3.95E−07 | 0.00018343 | 6.30981665 |
| 1438004_at | Papd7 | 1.15866963 | 8.7651013 | 5.72747713 | 4.29E−07 | 0.00019722 | 6.23364804 |
| 1453757_at | Herc6 | 1.25111898 | 9.09811412 | 5.64424695 | 5.83E−07 | 0.00026558 | 5.95039163 |
| 1456890_at | Ddx58 | 1.20405595 | 8.33433728 | 5.63983971 | 5.93E−07 | 0.00026723 | 5.93541754 |
| 1416697_at | Dpp4 | 1.1097733 | 9.72105276 | 5.63576616 | 6.01E−07 | 0.00026859 | 5.92157948 |
| 1422006_at | Eif2ak2 | 1.23465729 | 9.21616067 | 5.61858421 | 6.41E−07 | 0.00028336 | 5.86323608 |
| 1424617_at | Ifi35 | 1.10413772 | 12.2660487 | 5.60315363 | 6.78E−07 | 0.00029704 | 5.8108739 |
| 1426278_at | Ifi27l2a | 1.13356673 | 13.6135311 | 5.55624326 | 8.06E−07 | 0.00034964 | 5.65189199 |
| 1452676_a_a | LOC1005051 [symbol] | 1.11295467 | 9.74168879 | 5.53659908 | 8.67E−07 | 0.00037225 | 5.58541026 |
| 1451852_at | Tcstv3 | 1.22404052 | 6.8712238 | 5.52210287 | 9.14E−07 | 0.00038891 | 5.53638704 |
| 1449366_at | Mmp8 | 1.22832664 | 9.31371883 | 5.50428037 | 9.76E−07 | 0.00041133 | 5.47615784 |
| 1425405_a_a | Adar | 1.10728732 | 11.4786191 | 5.48996899 | 1.03E−06 | 0.0004295 | 5.42782879 |
| 1429570_at | Mlkl | 1.20072042 | 8.28523783 | 5.48602776 | 1.04E−06 | 0.00043175 | 5.4145249 |
| 1456288_at | Slfn5 | 1.32340243 | 9.39871024 | 5.47997663 | 1.07E−06 | 0.00043504 | 5.3941035 |
| 1417961_a_a | Trim30a | 1.17912824 | 12.8108758 | 5.47899844 | 1.07E−06 | 0.00043504 | 5.39080284 |
| 1444405_at | Pydc4 | 1.32066484 | 5.20069324 | 5.43876125 | 1.24E−06 | 0.0004952 | 5.25516142 |
| 1435454_a_a | Helz2 | 1.13249361 | 12.5204465 | 5.43239671 | 1.27E−06 | 0.00050243 | 5.23372972 |
| 1430530_s_a | Nmral1 | 1.17435967 | 9.31355989 | 5.37375224 | 1.57E−06 | 0.00061706 | 5.03656379 |
| 1438148_at | Cxcl3 | 1.69044193 | 7.09902456 | 5.35135811 | 1.71E−06 | 0.00065812 | 4.96142512 |
| 1422141_s_a | Csprs /// Gm [symbol] | 1.19436756 | 7.71681771 | 5.33738357 | 1.80E−06 | 0.00068662 | 4.91458012 |
| 1418116_at | Tor1aip2 | 1.08075944 | 10.7447031 | 5.32686698 | 1.87E−06 | 0.00070743 | 4.87934914 |
| 1425065_at | Oas2 | 1.28607157 | 10.4532534 | 5.32376691 | 1.89E−06 | 0.0007095 | 4.86896748 |
| 1436778_at | Cybb | 1.1006634 | 12.2766584 | 5.29025539 | 2.13E−06 | 0.00079482 | 4.75685109 |
| 1428346_at | Trafd1 | 1.15659445 | 11.2650229 | 5.28308824 | 2.19E−06 | 0.0008091 | 4.73289875 |
| 1440522_at | Chic1 | 1.11707615 | 8.8938542 | 5.26963652 | 2.30E−06 | 0.00084269 | 4.68796879 |
| 1418580_at | Rtp4 | 1.16283443 | 12.43898 | 5.25438045 | 2.43E−06 | 0.00088346 | 4.63705234 |
| 1425374_at | Oas3 | 1.32020993 | 10.3330288 | 5.23952375 | 2.56E−06 | 0.00092489 | 4.58751022 |
| 1435840_x_a | BC147527 | 1.21647215 | 7.14546794 | 5.20034393 | 2.95E−06 | 0.00104904 | 4.45705855 |
| 1459913_at | Tnfsf10 | 1.10617704 | 8.60982005 | 5.19467099 | 3.02E−06 | 0.00106241 | 4.43819454 |
| 1418346_at | Insl6 | 1.1375775 | 8.67983728 | 5.17515811 | 3.24E−06 | 0.00113118 | 4.3733569 |
| 1440299_at | Mb21d1 | 1.08850433 | 8.80638297 | 5.10764055 | 4.13E−06 | 0.0014208 | 4.14959418 |
| 1452306_at | Zfyve26 | 1.08114798 | 10.6819271 | 5.09739051 | 4.28E−06 | 0.00146298 | 4.11570542 |
| 1425008_a_a | Gm16340 // [symbol] | 1.14711446 | 8.2805057 | 5.07062975 | 4.71E−06 | 0.00159849 | 4.02733237 |
| 1426971_at | Uba7 | 1.0921264 | 11.5979875 | 5.05902098 | 4.91E−06 | 0.00165404 | 3.98904342 |
| 1427102_at | Slfn4 | 1.4307997 | 10.9094731 | 5.02506971 | 5.55E−06 | 0.00184054 | 3.87722913 |
| 1447272_s_a | Atp10a | 1.16573994 | 8.48146745 | 5.00861675 | 5.89E−06 | 0.00191674 | 3.82313389 |
| 1452161_at | Tiparp | 1.12341519 | 9.30889526 | 5.00763194 | 5.91E−06 | 0.00191674 | 3.81989785 |
| 1419668_at | Sgcb | 1.11311815 | 8.08482712 | 4.99202128 | 6.25E−06 | 0.00201223 | 3.7686309 |
| 1452973_at | Ppm1k | 1.06538857 | 10.5472337 | 4.97566869 | 6.62E−06 | 0.00211808 | 3.71498595 |
| 1440866_st | Eif2ak2 | 1.27644426 | 8.72230965 | 4.96189632 | 6.96E−06 | 0.00220906 | 3.66985249 |
| 1425225_at | Fcgr4 | 1.15148701 | 7.97090773 | 4.94964494 | 7.27E−06 | 0.00229151 | 3.62973994 |
| 1428843_at | 5-Mar | 1.06276216 | 11.9004332 | 4.89939887 | 8.69E−06 | 0.00270208 | 3.46559327 |
| 1455500_at | Rnf213 | 1.18104388 | 12.9664015 | 4.8925931 | 8.90E−06 | 0.00273916 | 3.44340567 |
| 1450322_s_a | Slfn3 /// Slfn [symbol] | 1.32138137 | 7.84711588 | 4.8916997 | 8.93E−06 | 0.00273916 | 3.4404939 |
| 1419569_a_a | Isg20 | 1.21173809 | 11.9296506 | 4.86452698 | 9.83E−06 | 0.00299161 | 3.35202465 |
| 1451161_a_a | Emr1 | 1.18065879 | 9.48930222 | 4.86302162 | 9.88E−06 | 0.00299161 | 3.3471287 |
| 1452348_s_a | Ifi204 /// Ifi2 [symbol] | 1.22015123 | 10.6929234 | 4.85654465 | 1.01E−05 | 0.00304061 | 3.32606974 |
| 1451564_at | Parp14 | 1.13986145 | 12.0654567 | 4.85172709 | 1.03E−05 | 0.00307242 | 3.31041282 |
| 1418244_at | Naa20 | 1.08893101 | 11.1644701 | 4.76808963 | 1.38E−05 | 0.00407322 | 3.03951865 |
| 1436899_at | Zufsp | 1.09784747 | 11.1470022 | 4.74873354 | 1.48E−05 | 0.00433173 | 2.97708107 |
| 1419599_s_a | Ms4a6d | 1.33981893 | 8.57140996 | 4.74664377 | 1.49E−05 | 0.0043355 | 2.97034589 |
| 1443621_at | Xaf1 | 1.16509294 | 7.24967566 | 4.74194912 | 1.51E−05 | 0.00437932 | 2.95521955 |
| 1436472_at | Slfn9 | 1.11910501 | 8.38309804 | 4.73923193 | 1.53E−05 | 0.00439313 | 2.94646731 |
| 1453562_a_a | Nmral1 | 1.15341413 | 9.19098832 | 4.73002801 | 1.58E−05 | 0.00450863 | 2.91683543 |
| 1454809_at | Ncoa7 | 1.09311088 | 10.3983869 | 4.7257237 | 1.60E−05 | 0.00454843 | 2.90298546 |
| 1436779_at | Cybb | 1.0941996 | 11.6457179 | 4.7126236 | 1.68E−05 | 0.00473241 | 2.86086374 |
| 1426970_a_a | Uba7 | 1.07523655 | 12.3984639 | 4.68910631 | 1.82E−05 | 0.00510655 | 2.78536262 |
| 1434139_at | Parp11 | 1.09338753 | 10.840668 | 4.6805127 | 1.88E−05 | 0.00522983 | 2.75781068 |
| 1452178_at | Parp10 /// P [symbol] | 1.10641059 | 11.7358548 | 4.66046456 | 2.01E−05 | 0.00552329 | 2.6936131 |
| 1417516_at | Ddit3 | 1.07223921 | 9.94848573 | 4.66039944 | 2.02E−05 | 0.00552329 | 2.69340478 |
| 1459973_x_a | Dpp4 | 1.14507385 | 7.63353457 | 4.65962457 | 2.02E−05 | 0.00552329 | 2.69092573 |
| 1422140_at | Gm7609 | 1.16864971 | 8.39169472 | 4.63609019 | 2.19E−05 | 0.00595944 | 2.61571222 |
| 1417685_at | Ankfy1 | 1.06791545 | 11.0934338 | 4.61188057 | 2.39E−05 | 0.00640602 | 2.53850302 |
| 1423627_at | Nqo1 | 1.16457475 | 6.67427154 | 4.59518149 | 2.53E−05 | 0.00674846 | 2.48534355 |
| 1451755_a_a | Apobec1 | 1.07861266 | 9.62528811 | 4.56307937 | 2.83E−05 | 0.00749855 | 2.38337634 |
| 1430700_a_a | Pla2g7 | 1.18443125 | 10.1013856 | 4.56135068 | 2.84E−05 | 0.00749945 | 2.37789398 |
| 1438948_x_a | Tspo | 1.10391642 | 11.5941786 | 4.5571403 | 2.89E−05 | 0.0075653 | 2.36454478 |
| 1450291_s_a | Ms4a4c | 1.12154489 | 12.4861899 | 4.55455972 | 2.91E−05 | 0.00758904 | 2.35636554 |
| 1419879_s_a | Trim25 | 1.09549971 | 12.1244381 | 4.5525076 | 2.93E−05 | 0.00759918 | 2.34986266 |
| 1428615_at | Lpar6 | 1.07528364 | 9.93379264 | 4.54743285 | 2.98E−05 | 0.00768686 | 2.33378681 |
| 1416695_at | Tspo | 1.0780893 | 12.4808237 | 4.54588597 | 3.00E−05 | 0.00768686 | 2.32888813 |
| 1424857_a_a | Trim34a | 1.11791554 | 10.7165118 | 4.53973664 | 3.06E−05 | 0.00780763 | 2.30942125 |
| 1446457_at | Ddx58 | 1.16007779 | 8.02122625 | 4.53347715 | 3.13E−05 | 0.00793348 | 2.28961716 |
| 1442640_at | — | 1.2168656 | 9.07831829 | 4.51929521 | 3.29E−05 | 0.00828496 | 2.24479093 |
| 1449078_at | St3gal6 | 1.09165835 | 10.7727541 | 4.51684697 | 3.32E−05 | 0.0083088 | 2.23705863 |
| 1449591_at | Casp4 | 1.10948228 | 11.1338558 | 4.50971919 | 3.40E−05 | 0.00846848 | 2.2145572 |
| 1418030_at | Slco3a1 | 1.08661497 | 9.54424904 | 4.49526041 | 3.57E−05 | 0.00880366 | 2.16895999 |
| 1416871_at | Adam8 | 1.1193914 | 9.62857594 | 4.48900903 | 3.65E−05 | 0.00894624 | 2.14926531 |
| 1454668_at | Ubr4 | 1.12814814 | 11.184894 | 4.48414411 | 3.71E−05 | 0.0089994 | 2.1339469 |
| 1442201_at | — | 1.1432126 | 9.81756401 | 4.47986246 | 3.77E−05 | 0.00908403 | 2.12047102 |
| 1436032_at | — | 1.21380001 | 11.1380493 | 4.47297003 | 3.86E−05 | 0.00925232 | 2.09878995 |
| 1454169_a_a | Epsti1 | 1.12897335 | 10.1539562 | 4.45618995 | 4.09E−05 | 0.00974916 | 2.04606725 |
| 1434601_at | Amigo2 | 1.13289174 | 9.38089015 | 4.42595648 | 4.53E−05 | 0.01075628 | 1.95129594 |
| 1448576_at | Il7r | 1.10438001 | 9.35743301 | 4.3992231 | 4.96E−05 | 0.01166251 | 1.86773693 |
| 1421550_a_a | Trim34a /// [symbol] | 1.12211108 | 9.72957463 | 4.39764723 | 4.99E−05 | 0.01166465 | 1.86281845 |
| 1415713_a_a | Ddx24 | 1.06449185 | 11.5232541 | 4.37956906 | 5.31E−05 | 0.01234241 | 1.80645151 |
| 1419667_at | Sgcb | 1.12646472 | 8.33509646 | 4.34873052 | 5.90E−05 | 0.01363746 | 1.71054358 |
| 1455227_at | Nceh1 | 1.08446292 | 9.91489676 | 4.34099563 | 6.05E−05 | 0.01392902 | 1.68653707 |
| 1418612_at | Slfn1 | 1.09272571 | 12.96439 | 4.32568503 | 6.38E−05 | 0.01459714 | 1.63907653 |
| 1452318_a_a | Hspa1b | 1.19057538 | 7.25960902 | 4.30400323 | 6.86E−05 | 0.01562991 | 1.57200028 |
| 1427127_x_a | Hspa1b | 1.21832745 | 7.30468517 | 4.29624366 | 7.04E−05 | 0.01588217 | 1.54803323 |
| 1419598_at | Ms4a6d | 1.30010562 | 8.68960331 | 4.29561853 | 7.06E−05 | 0.01588217 | 1.54610327 |
| 1448303_at | Gpnmb | 1.22944041 | 8.9049785 | 4.29482104 | 7.08E−05 | 0.01588217 | 1.54364139 |
| 1435660_at | LOC664787 | 1.08388982 | 8.74033836 | 4.29173015 | 7.15E−05 | 0.01596949 | 1.5341017 |
| 1422511_a_a | Ogfr | 1.07234468 | 12.3938197 | 4.24109747 | 8.48E−05 | 0.01875548 | 1.37829561 |
| 1437176_at | Nlrc5 | 1.07682641 | 12.3735454 | 4.23387901 | 8.69E−05 | 0.01912232 | 1.35615536 |
| 1436633_at | Gm11772 | 1.14601474 | 4.72784952 | 4.22090965 | 9.08E−05 | 0.01980988 | 1.316422 |
| 1451082_at | Ftsjd2 /// R [symbol] | 1.06640728 | 11.3628274 | 4.2204656 | 9.09E−05 | 0.01980988 | 1.31506267 |
| 1425974_a_a | Trim25 | 1.08480215 | 11.7657133 | 4.20895728 | 9.45E−05 | 0.02049028 | 1.27985683 |
| 1417876_at | Fcgr1 | 1.2323332 | 7.0806629 | 4.19894373 | 9.77E−05 | 0.0210878 | 1.24926191 |
| 1457069_at | Ascc3 | 1.11288948 | 9.43740297 | 4.19582127 | 9.87E−05 | 0.02120788 | 1.23972903 |
| 1429831_at | Pik3ap1 | 1.06680759 | 12.4737143 | 4.1806008 | 0.0001039 | 0.0222095 | 1.19331067 |
| 1449538_a_a | Gcnt1 | 1.11915617 | 8.47448492 | 4.14722631 | 0.00011614 | 0.02447684 | 1.09181994 |
| 1422005_at | Eif2ak2 | 1.19563857 | 9.53538322 | 4.12771094 | 0.00012393 | 0.02599698 | 1.03266263 |
| 1449875_s_a | H2-T10 /// H [symbol] | 1.04842787 | 12.8867189 | 4.11331035 | 0.00013 | 0.02714397 | 0.98909995 |
| 1418722_at | Ngp | 1.11092189 | 12.7881149 | 4.10510418 | 0.00013359 | 0.02776429 | 0.96431022 |
| 1426133_a_ [symbol] | Mitd1 | 1.10636222 | 10.1667831 | 4.07764709 | 0.00014629 | 0.03009875 | 0.88154944 |
| 1424354_at | Tmem140 | 1.11754478 | 9.34810202 | 4.07577197 | 0.0001472 | 0.03009875 | 0.87590785 |
| 1423052_at | Arf4 | 1.05369833 | 12.3484921 | 4.06902312 | 0.00015052 | 0.03046771 | 0.85561388 |
| 1456251_x_a | Tspo | 1.1023499 | 11.1159008 | 4.06877418 | 0.00015065 | 0.03046771 | 0.85486562 |
| 1423986_a_a | Shisa5 | 1.04210813 | 14.2786011 | 4.03888346 | 0.00016625 | 0.03347242 | 0.76519512 |
| 1428378_at | Zc3hav1 | 1.08752006 | 10.7288018 | 4.02875397 | 0.00017188 | 0.0343001 | 0.73488508 |
| 1420401_a_a | Ramp3 | 1.12512462 | 9.98404572 | 4.00377593 | 0.00018656 | 0.0370668 | 0.66031463 |
| 1460657_at | Wnt10a | 1.12684061 | 8.35987776 | 3.98317991 | 0.00019958 | 0.03947847 | 0.59901024 |
| 1424444_a_a | 1600014C10 [symbol] | 1.09898446 | 11.5959589 | 3.98018984 | 0.00020154 | 0.03969221 | 0.59012418 |
| 1428444_at | Asb2 | 1.13292232 | 8.24932226 | 3.95719355 | 0.00021725 | 0.04241625 | 0.52190117 |
| 1439814_at | Atp8b4 | 1.11510444 | 9.33960673 | 3.95146957 | 0.00022134 | 0.04302868 | 0.50495269 |
| 1429588_at | 2810474O19 [symbol] | 1.05637397 | 11.2428969 | 3.93455457 | 0.00023387 | 0.04526887 | 0.4549451 |
| 1433617_s_a | B4galt5 | 1.11846333 | 10.5599476 | 3.92169381 | 0.00024385 | 0.04679878 | 0.41700096 |
| 1431296_at | Gpr15 | 1.14301562 | 6.60205175 | 3.9193265 | 0.00024573 | 0.04695995 | 0.41002381 |
| 1454757_s_a | Ifi27l1 | −1.1515249 | 11.2428344 | −6.5569053 | 1.92E−08 | 1.57E−05 | 9.09173948 |
| 1415837_at | Klk1 | −1.4082454 | 7.5804401 | −6.3275656 | 4.55E−08 | 3.16E−05 | 8.29680484 |
| 1452956_a_a | Ifi27l1 | −1.1424326 | 10.7927481 | −5.9606126 | 1.80E−07 | 9.56E−05 | 7.03136178 |
| 1448107_x_a | Klk1 | −1.3445567 | 7.49119412 | −5.9058876 | 2.21E−07 | 0.00011452 | 6.84359495 |
| 1439771_s_a | D13ERTD608 [symbol] | −1.2546842 | 8.86787505 | −5.8507689 | 2.71E−07 | 0.00013591 | 6.65478212 |
| 1415823_at | Scd2 | −1.3537166 | 10.2235672 | −5.1396945 | 3.68E−06 | 0.00127567 | 4.25571056 |
| 1442544_at | Ighm | −1.1737796 | 9.42424035 | −5.0083987 | 5.89E−06 | 0.00191674 | 3.82241733 |
| 1415824_at | Scd2 | −1.3093243 | 10.9433795 | −4.6149411 | 2.36E−05 | 0.00637618 | 2.54825457 |
| 1415822_at | Scd2 | −1.2780512 | 11.8313256 | −4.5073901 | 3.43E−05 | 0.00848983 | 2.20720796 |
| 1429100_at | Ccdc71l | −1.0849357 | 10.7147161 | −4.4868075 | 3.68E−05 | 0.00896553 | 2.14233223 |
| 1427756_x_a | Ighm | −1.1707153 | 9.14115987 | −4.4212385 | 4.61E−05 | 0.01087406 | 1.93653267 |
| 1449325_at | Fads2 | −1.1644392 | 8.52583689 | −4.1780844 | 0.00010478 | 0.02229136 | 1.18564443 |
| 1416188_at | Gm2a | −1.0836296 | 10.9313169 | −4.163994 | 0.00010983 | 0.02325521 | 1.1427593 |
| 1424567_at | Tspan2 | −1.1339633 | 9.59951853 | −4.0339051 | 0.00016899 | 0.03387392 | 0.75029377 |
| 1436131_at | Siglech | −1.1248775 | 7.91450702 | −3.9583983 | 0.0002164 | 0.04241625 | 0.52546999 |

[symbol]

indicates data missing or illegible when filed

### Polimunol and Copaxone® Comparison: Differentially Expressed Pathways, Under Copaxone® Immunization

After imposing a conservative fold change filter of |FC|≧1.2, 73 probesets are found to be upregulated by Polimunol relative to Copaxone® (medium-corrected, in Copaxone®-immunized mice), and these probesets enrich for 22 pathways, including immune response and response to virus. These pathways may be relevant to Copaxone's mechanism of action; several of these pathways are also enriched among probesets modulated by Copaxone® relative to control (e.g., immune response and immune system process). A total of 6 downregulated probesets are detected, which do not enrich for pathways. The upregulated pathways are depicted in FIG. 30 and the full list of pathways enriched among top probesets upregulated by Polimunol relative to Copaxone® is provided in Table 21.

TABLE 21
Pathways enriched among top probesets upregulated by
Polimunol relative to Copaxone ® (in Copaxone ® -immunized mice).

| Category | Term | Count | Pvalue | Fold Enrichm | Benjamini |
|---|---|---|---|---|---|
| GOTERM_BP_ALL | GO: 0006955~immune response | 15 | 4.38E−11 | 9.95074106 | 1.98E−08 |
| GOTERM_BP_ALL | GO: 0009615~response to virus | 8 | 8.16E−10 | 38.2602137 | 1.84E−07 |
| GOTERM_BP_ALL | GO: 0002376~immune system process | 15 | 8.29E−08 | 5.57817311 | 9.35E−06 |
| GOTERM_BP_ALL | GO: 0051707~response to other organism | 9 | 8.19E−08 | 14.9261116 | 1.23E−05 |
| GOTERM_BP_ALL | GO: 0050896~response to stimulus | 21 | 1.38E−07 | 3.37128932 | 1.25E−05 |
| GOTERM_BP_ALL | GO: 0009607~response to biotic stimulus | 9 | 1.27E−06 | 10.4567109 | 9.54E−05 |
| GOTERM_BP_ALL | GO: 0051704~multi-organism process | 9 | 1.63E−06 | 10.113868 | 0.00010525 |
| KEGG_PATHWAY | mmu04622: RIG-1-like receptor signaling pathway | 5 | 1.47E−05 | 27.2429078 | 0.00023467 |
| KEGG_PATHWAY | mmu04623: Cytosolic DNA-sensing pathway | 4 | 0.00031378 | 25.6083333 | 0.00250745 |
| GOTERM_BP_ALL | GO: 0045087~innate immune response | 5 | 0.00030987 | 14.6891892 | 0.01731968 |
| GOTERM_MF_ALL | GO: 0001882~nucleoside binding | 16 | 0.00118891 | 2.41112097 | 0.02621825 |
| GOTERM_MF_ALL | GO: 0032559~adenyl ribonucleotide binding | 16 | 0.00065908 | 2.54969114 | 0.02901942 |
| GOTERM_MF_ALL | GO: 0001883~purine nucleoside binding | 16 | 0.00112323 | 2.42429649 | 0.02967043 |
| GOTERM_BP_ALL | GO: 0006952~defense response | 7 | 0.00060685 | 6.34158828 | 0.02996091 |
| GOTERM_MF_ALL | GO: 0030554~adenyl nucleotide binding | 16 | 0.00106069 | 2.4376168 | 0.03492727 |
| GOTERM_MF_ALL | GO: 0032553~ribonucleotide binding | 17 | 0.00211744 | 2.18735104 | 0.03976457 |
| GOTERM_MF_ALL | GO: 0032555~purine ribonucleotide binding | 17 | 0.00211744 | 2.18735104 | 0.03976457 |
| GOTERM_MF_ALL | GO: 0005524~ATP binding | 16 | 0.00061962 | 2.56442924 | 0.04067708 |
| GOTERM_MF_ALL | GO: 0001730~2′-5′-oligoadenylate synthetase activity | 3 | 0.0003357 | 99.8204082 | 0.04399453 |
| GOTERM_MF_ALL | GO: 0017076~purine nucleotide binding | 17 | 0.00310625 | 2.1090566 | 0.04526422 |
| GOTERM_MF_ALL | GO: 0016779~nucleotidyltransferase activity | 5 | 0.00284393 | 8.23600727 | 0.04658368 |
| GOTERM_MF_ALL | GO: 0003723~RNA binding | 10 | 0.00371417 | 3.08659271 | 0.04863977 |

### Polimunol/Copaxone® Comparison: Differentially Expressed Pathways, Polimunol Immunization

In Polimunol-immunized mice, after imposing a fold change filter of |FC|≧1.2, 77 probesets are upregulated by Polimunol relative to Copaxone® (medium-corrected, in Polimunol-immunized mice), and these probesets enrich for 10 pathways. These pathways are similar to those seen in the comparison for Copaxone®-immunized mice, and include pathways relevant to Copaxone's mechanism of action such as immune response and immune system process pathways, as well as potentially concerning pathways such as response to virus. 22 downregulated probesets are detected, which do not enrich for pathways. The upregulated pathways are depicted in FIG. 31 and provided in Table 22.

TABLE 22
Pathways enriched among top probesets upregulated by
Polimunol relative to Copaxone ® (in Polimunol-immunized mice).

| Category | Term | Count | Pvalue | Fold Enrichm | Benjamini |
|---|---|---|---|---|---|
| GOTERM_BP_ALL | GO: 0009615~response to virus | 8 | 3.62E−09 | 31.4583979 | 1.93E−06 |
| GOTERM_BP_ALL | GO: 0009607~response to biotic stimulus | 11 | 4.50E−08 | 10.508349 | 1.20E−05 |
| GOTERM_BP_ALL | GO: 0051707~response to other organism | 9 | 4.30E−07 | 12.2725806 | 5.73E−05 |
| GOTERM_BP_ALL | GO: 0050896~response to stimulus | 23 | 3.87E−07 | 3.03594414 | 6.88E−05 |
| GOTERM_BP_ALL | GO: 0051704~multi-organism process | 10 | 7.59E−07 | 9.23982999 | 8.09E−05 |
| GOTERM_BP_ALL | GO: 0006955~immune response | 12 | 9.89E−07 | 6.54537634 | 8.78E−05 |
| GOTERM_BP_ALL | GO: 0002376~immune system process | 12 | 0.0002277 | 3.66919831 | 0.01719059 |
| GOTERM_CC_ALL | GO: 0044421~extracellular region part | 7 | 0.00098754 | 5.86744639 | 0.03589677 |
| GOTERM_CC_ALL | GO: 0005576~extracellular region | 10 | 0.00051394 | 4.06619385 | 0.03732702 |
| GOTERM_CC_ALL | GO: 0005615~extracellular space | 6 | 0.00178939 | 6.62813102 | 0.04321627 |

### Polimunol and Copaxone Comparison: Discussion

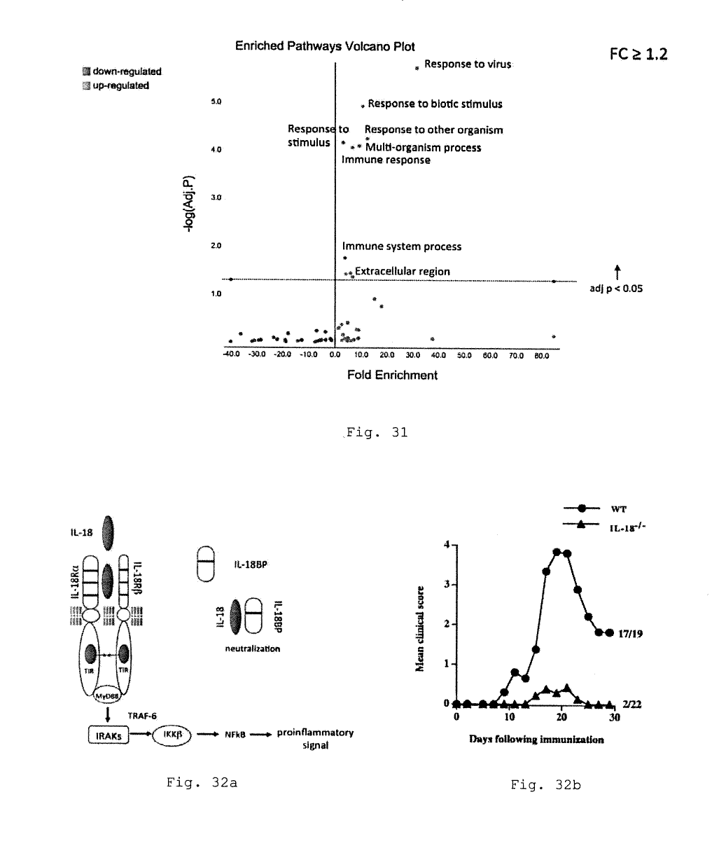

The fact that Polimunol modulates IL18 to a significantly different extent than Copaxone®, consistently, regardless of immunization agent is noteworthy and has potential implications for both safety and efficacy. IL18 is important for T helper cell differentiation, and IL18 levels are higher in serum from MS patients versus controls, as well as acute versus stable MS (Nicoletti et al, 2001). The signaling pathway for IL-18 appears in FIG. 32a. IL18 also induces IFNg expression and has been implicated in MS immunopathogenesis (Losy et al, 2001). IL18 genetic variants have further been shown to affect MS risk (Celik et al, 2014; Thompson et al, 2007). Moreover, mice deficient for IL18 are resistant to EAE (Shi et al, J. Immunol., 2000) as shown in FIG. 32b.

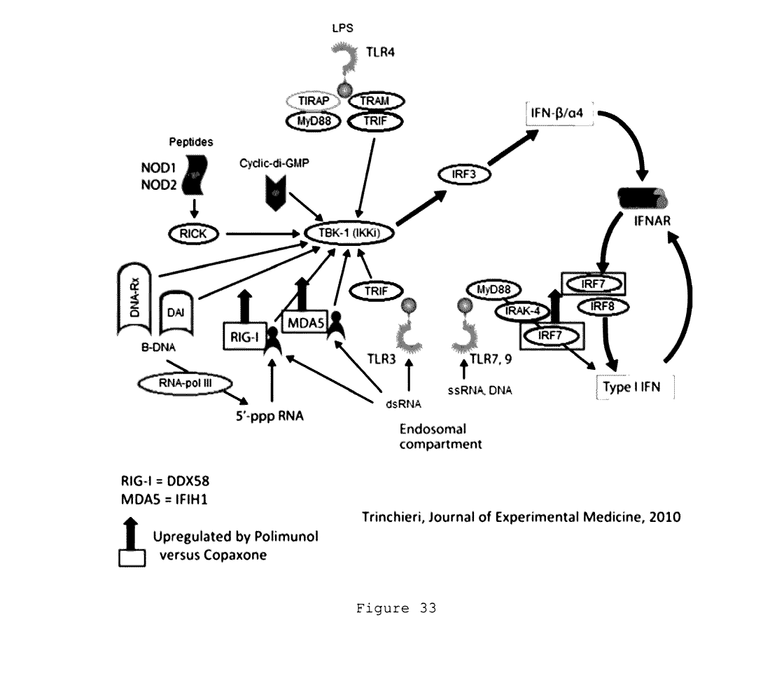

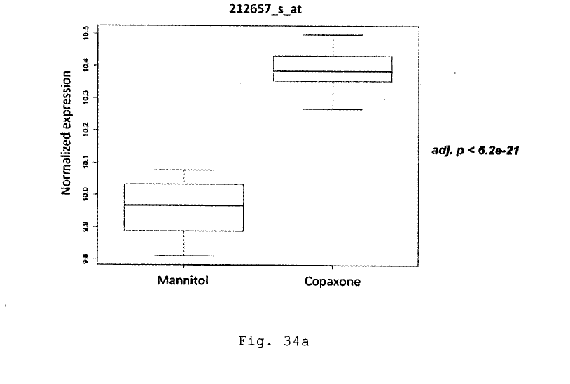

Intriguingly, a number of interferon-related genes are upregulated by Polimunol relative to Copaxone® in this model system. FIG. 33 shows illustrated pathways of IFN Type I production (Trinchieri et al, J Exp Med, 2010), with overlay indicating genes including RIG-I, MDA5, and IRF7 upregulated by Polimunol versus Copaxone. As the figure indicates, the upregulation of these genes would be consistent with an increase in type I interferon production.

At the pathway level, the fact that a variety of immune-related pathways are enriched among the differentially expressed probesets, including “immune system process” and “response to virus,” and that differential expression is seen for multiple genes affecting interferon signaling (e.g. leading to the significant enrichment of the RIG-I-like receptor signaling pathway in the comparison between Polimunol and Copaxone® in Copaxone-immunized mice), raises serious concerns for safety and efficacy.

### Example 11

### Differences in Gene Expression Induced by Polimunol Versus Branded GA in Human Monocyte (THP-1) Gene Expression Studies

The immunized mouse splenocyte model system has proved robust and informative in examining Copaxone®'s mode of action, as well as in differentiating between seemingly similar FOGAs and Copaxone® [Bakshi et al, 2013; Towfic et al, 2014; FDA-2014-P-0933-0001 available at www.regulations.gov/#?documentDetail;D=FDA-2014-P-0933-0001, and also described herein; and FDA-2013-P-1641-0001 available at www.regulations.gov/#?documentDetail;D=FDA-2013-P-1641-000l, and also described herein. To follow up on these findings and test a human-source model system, THP-1 cells were utilized. The research community uses this human monocyte cell line to model the behavior of antigen-presenting cells. As described in further detail in the methods section below, THP-1 cells were utilized to (1) compare the genome-wide transcriptional impact of Copaxone® to that of a mannitol control, in order to shed additional light on Copaxone®'s mechanism of action (MoA), and to (2) compare the genome-wide transcriptional impact of Copaxone® to that of purported generics, including Polimunol. It is critical to methodologically pursue the MoA analyses first and independently of any subsequent investigations so as to ensure the validity and robustness of each experiment. To this end, at least three different batches of Copaxone are tested and analyzed in comparison to control and to prior similar studies.

The study was performed in two repeats identical in design, reagents, drug lots, and technicians, performed on different days. Six replicate samples for each condition were utilized in each of the two experimental runs for a total of twelve replicates per condition, making this study well powered to detect changes in expression levels across conditions.

### Copaxone®-Modulated Genes (MoA)

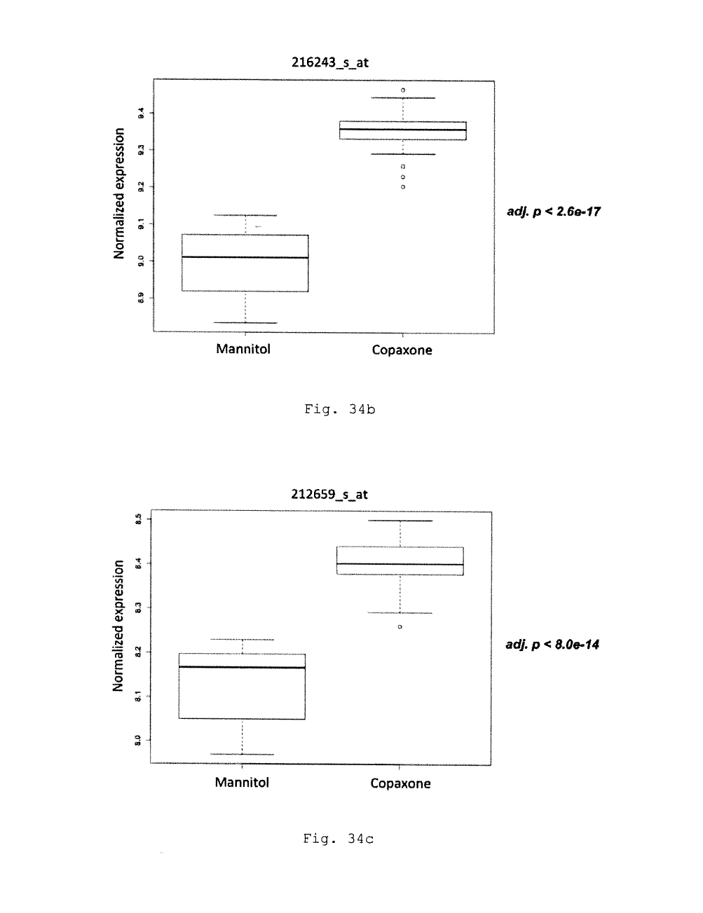

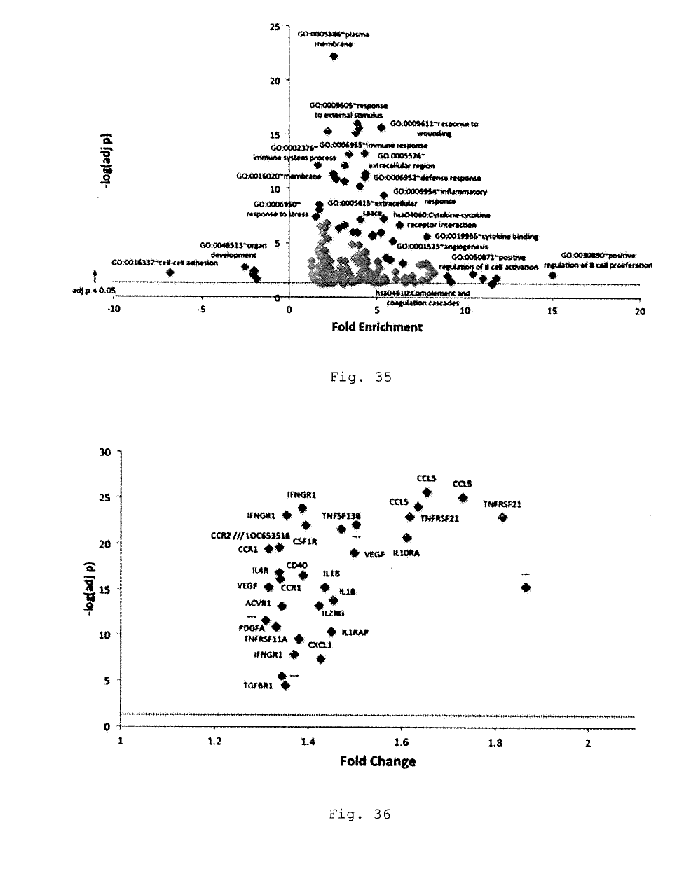

Copaxone® significantly upregulated 5296 probesets, and significantly downregulated 6819 probesets, out of >25,000 probesets called present on the chip. Consistent with previous studies (e.g. FDA-2014-P-0933-0001 available at www.regulations.gov/#?documentDetail;D=FDA-2014-P-0933-0001, and also described herein), the anti-inflammatory gene IL1RN was strongly upregulated by Copaxone® (all 3 IL1RN probesets that were called present in the study: FDR p values<6.2e-21, 2.6e-17, 8.0e-14); see FIG. 34a-c.

IL10 was not upregulated significantly, but may have been difficult to detect because the single probeset on the chip was not present. IL1ORA, representing the receptor necessary for IL10 signaling, was significantly upregulated (FDR p<1.7e-21), indicating this pathway upregulated by Copaxone®, as expected. These results are consistent with the results of a prior study presented earlier, of which the currently discussed data represents two independent confirmatory datasets (FDA-2014-P-0933-0001 available at www.regulations.gov/#?documentDetail;D=FDA-2014-P-0933-0001, and also described herein).

To test concordance with previous observations in the same model system, sets of top probesets modulated by Copaxone® were defined based on two prior THP-1 studies (FDA-2014-P-0933-0001 available at www.regulations.gov/#?documentDetail;D=FDA-2014-P-0933-0001, and also described herein), by taking the intersection of the top probesets (subject to fold change (FC) filter of 1.5, treating up and downregulated probesets separately) modulated by Copaxone® across both prior datasets (see Table 23). Significant enrichment of these sets by directionality was found in the present dataset among probesets upregulated by Copaxone® versus mannitol (FC≧1.5) and probesets downregulated by Copaxone® versus mannitol (FC≦−1.5) by the hypergeometric test: 2.4e-10 and 7.6e-11 for up and downregulated sets, respectively.

TABLE 23
Top probesets modulated by Copaxone ® are significantly
enriched among the top probesets modulated by
Copaxone ® in prior THP-1 studies.

| | In two prior | In current | |
|---|---|---|---|
| | THP-1 studies | study | Intersection |
| Probesets upregulated by | 149 | 156 | 94 |
| Copaxone versus mannitol | | | |
| with FC ≧ 1.5 | | | |
| Probesets downregulated | 15 | 28 | 9 |
| by Copaxone versus | | | |
| mannitol with FC ≦ −1.5 | | | |

### Copaxone®-Modulated Pathways

Among the top probesets upregulated by Copaxone® with FC≧1.3, 180 pathways were enriched. This includes immune response, immune system process, and cytokine-cytokine receptor interaction and regulation of B cell activation (as observed in prior THP-1 studies, e.g. FDA-2014-P-0933-0001 available at www.regulations.gov/#?documentDetail;D=FDA-2014-P-0933-0001, and also described herein) as well as coagulation, positive regulation of lymphocyte activation and proliferation, and positive regulation of B-cell activation and proliferation. The full list of pathways entiched among top probesets differentially expressed by Copaxone® relative to mannitol control in THP-1 cells is provided in Table 24.

TABLE 24
Pathways significantly enriched among top probesets
differently expressed by Copaxone ® relative to mannitol control in THP-1 cells.

| Category | Term | Count | Pvalue | Fold Enrichm | Benjamini |
|---|---|---|---|---|---|
| DOWN | | | | | |
| GOTERM_BP_ALL | GO: 0048513~organ development | 30 | 2.78E−06 | 2.52785617 | 0.00198109 |
| GOTERM_BP_ALL | GO: 0032502~developmental process | 44 | 2.37E−06 | 1.99322239 | 0.00337678 |
| GOTERM_BP_ALL | GO: 0048856~anatomical structure development | 36 | 1.55E−05 | 2.07315026 | 0.00550192 |
| GOTERM_BP_ALL | GO: 0016337~cell-cell adhesion | 10 | 1.38E−05 | 6.80689385 | 0.00654548 |
| GOTERM_BP_ALL | GO: 0048731~system development | 33 | 3.08E−05 | 2.11219642 | 0.00874514 |
| GOTERM_BP_ALL | GO: 0007275~multicellular organismal development | 37 | 8.63E−05 | 1.88932377 | 0.0203102 |
| UP | | | | | |
| GOTERM_CC_ALL | GO: 0005886~plasma membrane | 122 | 2.34E−25 | 2.51413927 | 5.85E−23 |
| GOTERM_CC_ALL | GO: 0031226~intrinsic to plasma membrane | 58 | 6.43E−19 | 3.81223099 | 8.04E−17 |
| GOTERM_BP_ALL | GO: 0009611~response to wounding | 44 | 2.00E−19 | 3.2102973 | 2.16E−16 |
| GOTERM_BP_ALL | GO: 0009605~response to external stimulus | 58 | 1.09E−19 | 3.93471319 | 2.34E−16 |
| GOTERM_BP_ALL | GO: 0050896~response to stimulus | 118 | 6.20E−19 | 2.15982642 | 4.45E−16 |
| GOTERM_CC_ALL | GO: 0005887~integral to plasma membrane | 56 | 7.55E−18 | 3.73686273 | 6.29E−16 |
| GOTERM_BP_ALL | GO: 0006955~immune response | 46 | 7.16E−17 | 4.268938 | 4.79E−14 |
| GOTERM_BP_ALL | GO: 0002376~immune system process | 59 | 7.00E−17 | 3.37823803 | 5.97E−14 |
| GOTERM_CC_ALL | GO: 0016020~membrane | 174 | 1.26E−14 | 1.56061376 | 6.33E−13 |
| GOTERM_CC_ALL | GO: 0005576~extracellular region | 58 | 1.03E−14 | 3.10216639 | 6.45E−13 |
| GOTERM_BP_ALL | GO: 0006932~defense response | 40 | 1.08E−14 | 4.30462838 | 3.86E−12 |
| GOTERM_CC_ALL | GO: 0044439~plasma membrane part | 76 | 9.39E−14 | 2.43596934 | 4.00E−12 |
| GOTERM_MF_ALL | GO: 0004888~transmembrane receptor activity | 39 | 4.19E−14 | 4.23233219 | 8.19E−12 |
| GOTERM_MF_ALL | GO: 0060089~molecular transducer activity | 69 | 3.91E−14 | 2.64236631 | 1.15E−11 |
| GOTERM_MF_ALL | GO: 0004871~signal transducer activity | 69 | 3.91E−14 | 2.64356631 | 1.15E−11 |
| GOTERM_MF_ALL | GO: 0004872~receptor activity | 56 | 3.49E−14 | 3.09677878 | 2.05E−11 |
| GOTERM_CC_ALL | GO: 0044421~extracellular region part | 37 | 1.44E−12 | 4.00069218 | 3.16E−11 |
| GOTERM_BP_ALL | GO: 0006934~inflammatory response | 28 | 1.16E−12 | 5.37828278 | 3.57E−10 |
| GOTERM_CC_ALL | GO: 0031234~intrinsic to membrane | 125 | 9.85E−11 | 1.63813557 | 3.08E−09 |
| GOTERM_CC_ALL | GO: 0016021~integral to membrane | 121 | 2.99E−10 | 1.63586081 | 8.31E−09 |
| GOTERM_CC_ALL | GO: 0044425~membrane part | 146 | 1.37E−09 | 1.49687764 | 3.43E−08 |
| KEGG_PATHWAY | hsa04060: Cytokine-cytokine receptor interaction | 21 | 4.90E−10 | 3.38268996 | 5.00E−08 |
| GOTERM_CC_ALL | GO: 0005615~extracellular space | 27 | 2.52E−09 | 4.02507822 | 5.72E−08 |
| GOTERM_BP_ALL | GO: 0006930~response to stress | 70 | 5.34E−10 | 2.13148534 | 1.44E−07 |
| GOTERM_BP_ALL | GO: 0042060~wound healing | 19 | 7.93E−10 | 6.27722433 | 1.90E−07 |
| GOTERM_BP_ALL | GO: 0050793~regulation of developmental process | 38 | 9.55E−10 | 3.11524781 | 2.06E−07 |
| GOTERM_BP_ALL | GO: 0031501~multicellular organismal process | 103 | 1.58E−09 | 1.72386846 | 3.09E−07 |
| GOTERM_BP_ALL | GO: 0031239~regulation of multicellular organismal process | 43 | 2.23E−09 | 2.76388129 | 4.03E−07 |
| GOTERM_BP_ALL | GO: 0048731~system development | 74 | 2.51E−09 | 1.99902 | 4.15E−07 |
| GOTERM_BP_ALL | GO: 0048314~blood vessel morphogenesis | 20 | 5.89E−09 | 5.24413041 | 9.07E−07 |
| GOTERM_BP_ALL | GO: 0048383~regulation of response to stimulus | 29 | 6.99E−09 | 3.58839948 | 9.41E−07 |
| GOTERM_BP_ALL | GO: 0040011~locomotion | 27 | 6.77E−09 | 3.82844041 | 9.71E−07 |
| GOTERM_BP_ALL | GO: 0007165~signal transduction | 79 | 1.11E−08 | 1.87343671 | 1.40E−06 |
| GOTERM_BP_ALL | GO: 0001568~blood vessel development | 21 | 1.27E−08 | 4.73204438 | 1.52E−06 |
| GOTERM_BP_ALL | GO: 0001944~vasculature development | 21 | 1.61E−08 | 4.68782736 | 1.82E−06 |
| GOTERM_MF_ALL | GO: 0019933~cytokine binding | 14 | 1.61E−08 | 7.82935604 | 2.36E−06 |
| GOTERM_BP_ALL | GO: 0065008~regulation of biological quality | 55 | 3.55E−08 | 2.18138206 | 3.82E−06 |
| GOTERM_BP_ALL | GO: 0001525~angiogenesis | 16 | 6.27E−08 | 5.87342628 | 6.43E−06 |
| GOTERM_BP_ALL | GO: 0048856~anatomical structure development | 75 | 9.87E−08 | 1.82463307 | 9.66E−06 |
| GOTERM_BP_ALL | GO: 0002682~regulation of immune system process | 23 | 1.35E−07 | 3.52970329 | 1.26E−05 |
| GOTERM_CC_ALL | GO: 0009986~cell surface | 20 | 6.73E−07 | 3.93630209 | 1.40E−05 |
| GOTERM_BP_ALL | GO: 0007275~multicellular organismal development | 80 | 3.39E−07 | 1.72409773 | 3.04E−05 |
| GOTERM_BP_ALL | GO: 0048513~organ development | 56 | 5.42E−07 | 1.99152773 | 4.67E−05 |
| GOTERM_BP_ALL | GO: 0032502~developmental process | 86 | 7.67E−07 | 1.64425345 | 6.11E−05 |
| GOTERM_BP_ALL | GO: 0048584~positive regulation of response to stimulus | 18 | 7.65E−07 | 4.30930732 | 6.33E−05 |
| GOTERM_BP_ALL | GO: 0002684~positive regulation of immune system process | 18 | 8.48E−07 | 4.27830511 | 6.52E−05 |
| GOTERM_BP_ALL | GO: 0016477~cell migration | 19 | 1.39E−06 | 3.92326521 | 0.00010335 |
| GOTERM_BP_ALL | GO: 0048318~positive regulation of biological process | 70 | 1.76E−06 | 1.74540498 | 0.00012637 |
| GOTERM_BP_ALL | GO: 0048519~negative regulation of biological process | 65 | 2.99E−06 | 1.77184118 | 0.00020737 |
| GOTERM_BP_ALL | GO: 0032101~regulation of response to external stimulus | 13 | 3.24E−06 | 5.50633714 | 0.00021167 |
| GOTERM_BP_ALL | GO: 0007168~cell surface receptor linked signal transduction | 43 | 3.17E−06 | 2.13308554 | 0.00021349 |
| GOTERM_BP_ALL | GO: 0048870~cell motility | 19 | 3.65E−06 | 3.67089141 | 0.00023142 |
| GOTERM_BP_ALL | GO: 0051674~localization of cell | 19 | 3.65E−06 | 3.67089142 | 0.00023142 |
| GOTERM_BP_ALL | GO: 0051707~response to other organism | 18 | 3.91E−06 | 3.83667362 | 0.00024052 |
| GOTERM_CC_ALL | GO: 0031012~extracellular matrix | 14 | 1.81E−05 | 4.34038261 | 0.00034771 |
| GOTERM_BP_ALL | GO: 0050878~regulation of body fluid levels | 12 | 5.88E−06 | 5.7457431 | 0.00035133 |
| GOTERM_BP_ALL | GO: 0006928~cell motion | 24 | 6.70E−06 | 2.92587656 | 0.00038982 |
| GOTERM_BP_ALL | GO: 0050776~regulation of immune response | 16 | 7.15E−06 | 4.09773926 | 0.00040481 |
| GOTERM_BP_ALL | GO: 0042127~regulation of cell proliferation | 34 | 8.23E−06 | 2.32085284 | 0.00043212 |
| GOTERM_BP_ALL | GO: 0022603~regulation of anatomical structure morphogenesis | 16 | 7.86E−06 | 4.06621819 | 0.000434 |
| GOTERM_BP_ALL | GO: 0042330~taxis | 13 | 8.15E−06 | 5.05287408 | 0.00043846 |
| GOTERM_BP_ALL | GO: 0006935~chemotaxis | 13 | 8.15E−06 | 5.05287408 | 0.00043846 |
| GOTERM_BP_ALL | GO: 0048646~anatomical structure formation involved in | 21 | 8.68E−06 | 3.18256183 | 0.00044506 |
| | morphogenesis | | | | |
| GOTERM_BP_ALL | GO: 0065007~biological regulation | 170 | 9.39E−06 | 1.25760499 | 0.00046996 |
| GOTERM_BP_ALL | GO: 0042221~response to chemical stimulus | 47 | 1.17E−05 | 1.94341311 | 0.00057284 |
| GOTERM_BP_ALL | GO: 0002703~regulation of leukocyte mediated immunity | 9 | 1.37E−05 | 7.82479488 | 0.00064059 |
| GOTERM_BP_ALL | GO: 0007626~locomotory behavior | 16 | 1.37E−05 | 3.88682621 | 0.00065406 |
| GOTERM_BP_ALL | GO: 0050865~regulation of cell activation | 14 | 1.49E−05 | 4.40506971 | 0.00068044 |
| GOTERM_MF_ALL | GO: 0019838~growth factor binding | 11 | 5.88E−06 | 6.46443198 | 0.00069034 |
| GOTERM_BP_ALL | GO: 0002822~regulation of adaptive immune response based on | 9 | 1.68E−05 | 7.62415911 | 0.00075234 |
| | somatic recombination of immune receptors built from immunoglobulin | | | | |
| | superfamily domains | | | | |
| GOTERM_BP_ALL | GO: 0002819~regulation of adaptive immune response | 9 | 2.04E−05 | 7.43355513 | 0.0008974 |
| GOTERM_BP_ALL | GO: 0030154~cell differentiation | 49 | 2.72E−05 | 1.848017258 | 0.00116853 |
| GOTERM_BP_ALL | GO: 0048522~positive regulation of cellular process | 62 | 3.31E−05 | 1.67486297 | 0.00139553 |
| GOTERM_BP_ALL | GO: 0050789~regulation of biological process | 162 | 3.82E−05 | 1.2502125 | 0.00158231 |
| GOTERM_BP_ALL | GO: 0010033~response to organic substance | 31 | 4.31E−05 | 2.24600094 | 0.00171764 |
| GOTERM_BP_ALL | GO: 0008284~positive regulation of cell proliferation | 21 | 4.26E−05 | 2.85513777 | 0.00172889 |
| GOTERM_BP_ALL | GO: 0045765~regulation of angiogenesis | 8 | 4.56E−05 | 8.00921765 | 0.00178429 |
| GOTERM_BP_ALL | GO: 0050867~positive regulation of cell activation | 11 | 4.77E−05 | 5.11856691 | 0.00183348 |
| GOTERM_CC_ALL | GO: 0005578~proteinaceous extracellular matrix | 12 | 0.0001102 | 4.24643494 | 0.00196599 |
| GOTERM_CC_ALL | GO: 0031982~vesicle | 28 | 0.00012722 | 2.234456653 | 0.00211822 |
| GOTERM_BP_ALL | GO: 0048869~cellular developmental process | 50 | 5.94E−05 | 1.77814977 | 0.00220286 |
| GOTERM_BP_ALL | GO: 0048545~response to steroid hormone stimulus | 13 | 5.85E−05 | 4.16984754 | 0.00220693 |
| GOTERM_BP_ALL | GO: 0051094~positive regulation of developmental process | 17 | 6.39E−05 | 3.24651091 | 0.00232928 |
| GOTERM_BP_ALL | GO: 0032879~regulation of localization | 25 | 0.00010654 | 2.4010191 | 0.00381602 |
| GOTERM_CC_ALL | GO: 0016023~cytoplasmic membrane-bounded vesicle | 24 | 0.0002448 | 2.32906958 | 0.00381824 |
| KEGG_PATHWAY | hsa04640: Hematopoietic cell lineage | 9 | 8.02E−05 | 6.02036055 | 0.00408452 |
| GOTERM_CC_ALL | GO: 0009897~external side of plasma membrane | 10 | 0.00031648 | 4.54973171 | 0.00464398 |
| GOTERM_CC_ALL | GO: 0031988~membrane-bounded vesicle | 24 | 0.00039301 | 2.25413972 | 0.00490148 |
| GOTERM_CC_ALL | GO: 0030141~secretory granule | 11 | 0.00038339 | 4.01420803 | 0.00503284 |
| GOTERM_CC_ALL | GO: 0031410~cytoplasmic vesicle | 26 | 0.00037285 | 2.16871498 | 0.00516605 |
| GOTERM_BP_ALL | GO: 0015718~monocarboxylic acid transport | 7 | 0.00015289 | 8.2595057 | 0.00538215 |
| GOTERM_BP_ALL | GO: 0048523~negative regulation of cellular process | 56 | 0.00015724 | 1.64309882 | 0.00544596 |
| GOTERM_BP_ALL | GO: 0002696~positive regulation of leukocyte activation | 10 | 0.00016618 | 4.93104818 | 0.00566357 |
| GOTERM_BP_ALL | GO: 0002694~regulation of leukocyte activation | 12 | 0.00018259 | 4.00460883 | 0.00612434 |
| GOTERM_BP_ALL | GO: 0050864~regulation of B cell activation | 7 | 0.00018795 | 7.97469516 | 0.00620684 |
| GOTERM_BP_ALL | GO: 0050871~positive regulation of B cell activation | 6 | 0.00019715 | 10.4330598 | 0.00641133 |
| GOTERM_BP_ALL | GO: 0042981~regulation of apoptosis | 35 | 0.00020205 | 1.94996762 | 0.00647234 |
| GOTERM_BP_ALL | GO: 0051240~positive regulation of multicellular organismal process | 14 | 0.00020826 | 3.42616532 | 0.00657273 |
| GOTERM_BP_ALL | GO: 0007186~G-protein coupled receptor protein signaling pathway | 19 | 0.00021787 | 2.70570014 | 0.00677572 |
| GOTERM_BP_ALL | GO: 0030890~positive regulation of B cell proliferation | 5 | 0.00022555 | 15.0172831 | 0.00691393 |
| GOTERM_BP_ALL | GO: 0043067~regulation of programmed cell death | 35 | 0.00024393 | 1.93043539 | 0.00737064 |
| GOTERM_BP_ALL | GO: 0051704~multi-organism process | 28 | 0.00024768 | 2.14135333 | 0.00737981 |
| GOTERM_BP_ALL | GO: 0080134~regulation of response to stress | 16 | 0.00025225 | 3.02061929 | 0.00741299 |
| GOTERM_BP_ALL | GO: 0009653~anatomical structure morphogenesis | 38 | 0.00025614 | 1.85991832 | 0.00742555 |
| GOTERM_BP_ALL | GO: 0010941~regulation of cell death | 35 | 0.00026015 | 1.92401131 | 0.00744133 |
| GOTERM_BP_ALL | GO: 0045087~innate immune response | 10 | 0.00029011 | 4.58861428 | 0.00808025 |
| GOTERM_BP_ALL | GO: 0001501~skeletal system development | 15 | 0.00028723 | 3.13652115 | 0.00810512 |
| GOTERM_BP_ALL | GO: 0050794~regulation of cellular process | 153 | 0.00029774 | 1.22749332 | 0.00818597 |
| GOTERM_BP_ALL | GO: 0007155~cell adhesion | 23 | 0.00035599 | 2.31669062 | 0.0096566 |
| GOTERM_BP_ALL | GO: 0022610~biological adhesion | 23 | 0.0003765 | 2.30964901 | 0.01008332 |
| GOTERM_BP_ALL | GO: 0002706~regulation of lymphocyte mediated immunity | 7 | 0.00039607 | 7.00806545 | 0.01047439 |
| GOTERM_BP_ALL | GO: 0002697~regulation of immune effector process | 9 | 0.00040369 | 4.95570342 | 0.01054539 |
| GOTERM_BP_ALL | GO: 0032680~regulation of tumor necrosis factor production | 6 | 0.00041457 | 9.01036985 | 0.0106985 |
| GOTERM_BP_ALL | GO: 0051270~regulation of cell motion | 13 | 0.00043338 | 3.38184485 | 0.01104886 |
| GOTERM_BP_ALL | GO: 0009607~response to biotic stimulus | 18 | 0.00048671 | 2.61975511 | 0.01225545 |
| GOTERM_BP_ALL | GO: 0048878~chemical homeostasis | 19 | 0.00051554 | 2.52097362 | 0.01282688 |
| GOTERM_BP_ALL | GO: 0051241~negative regulation of multicellular organismal process | 10 | 0.00053115 | 4.23564395 | 0.01306189 |
| GOTERM_BP_ALL | GO: 0031099~regeneration | 7 | 0.00055185 | 6.60760456 | 0.01341453 |
| GOTERM_BP_ALL | GO: 0001503~ossification | 9 | 0.00056526 | 4.71971754 | 0.01358496 |
| GOTERM_BP_ALL | GO: 0007596~blood coagulation | 8 | 0.00060782 | 5.39396291 | 0.01443964 |
| GOTERM_BP_ALL | GO: 0050817~coagulation | 8 | 0.00060782 | 5.39396291 | 0.01443964 |
| GOTERM_BP_ALL | GO: 0051251~positive regulation of lymphocyte activation | 9 | 0.00062947 | 4.64597198 | 0.01478703 |
| GOTERM_BP_ALL | GO: 0030193~regulation of blood coagulation | 5 | 0.00063688 | 11.7992938 | 0.01479853 |
| GOTERM_BP_ALL | GO: 0046903~secretion | 14 | 0.00066151 | 3.04297579 | 0.01520272 |
| GOTERM_BP_ALL | GO: 0060348~bone development | 9 | 0.00069943 | 4.57449547 | 0.0158979 |
| GOTERM_BP_ALL | GO: 0002700~regulation of production of molecular mediator of | 6 | 0.00077675 | 7.92912548 | 0.01745622 |
| | immune response | | | | |
| GOTERM_BP_ALL | GO: 0030888~regulation of B cell proliferation | 5 | 0.00084803 | 11.0126743 | 0.01846594 |
| GOTERM_BP_ALL | GO: 0050778~positive regulation of immune response | 10 | 0.00083992 | 3.98048468 | 0.01847785 |
| GOTERM_BP_ALL | GO: 0010876~lipid localization | 11 | 0.00083164 | 3.63418251 | 0.01848608 |
| GOTERM_BP_ALL | GO: 0001819~positive regulation of cytokine production | 9 | 0.00085816 | 4.43794336 | 0.01849764 |
| GOTERM_BP_ALL | GO: 0032103~positive regulation of response to external stimulus | 7 | 0.0008702 | 6.08595157 | 0.01856902 |
| GOTERM_BP_ALL | GO: 0007610~behavior | 17 | 0.00088888 | 2.57635958 | 0.01877795 |
| GOTERM_BP_ALL | GO: 0051179~localization | 80 | 0.00090886 | 1.39180717 | 0.01900988 |
| GOTERM_CC_ALL | GO: 0031091~platelet alpha granule | 6 | 0.0016609 | 6.78059772 | 0.01959452 |
| GOTERM_BP_ALL | GO: 0009615~response to virus | 9 | 0.00094771 | 4.37267949 | 0.01962413 |
| GOTERM_BP_ALL | GO: 0043065~positive regulation of apoptosis | 22 | 0.00097104 | 2.20253485 | 0.01972357 |
| GOTERM_BP_ALL | GO: 0001817~regulation of cytokine production | 12 | 0.00096317 | 3.30380228 | 0.01975149 |
| GOTERM_BP_ALL | GO: 0015849~organic acid transport | 10 | 0.00099852 | 3.88682621 | 0.02008682 |
| GOTERM_BP_ALL | GO: 0046942~carboxylic acid transport | 10 | 0.00099852 | 3.88682621 | 0.02008682 |
| GOTERM_BP_ALL | GO: 0007167~enzyme linked receptor protein signaling pathway | 16 | 0.00103032 | 2.64304183 | 0.02052865 |
| GOTERM_BP_ALL | GO: 0043068~positive regulation of programmed cell death | 22 | 0.00104554 | 2.18926657 | 0.02063801 |
| GOTERM_BP_ALL | GO: 0010942~positive regulation of cell death | 22 | 0.0010885 | 2.1826922 | 0.02128235 |
| GOTERM_BP_ALL | GO: 0007599~hemostasis | 8 | 0.00110252 | 4.8945219 | 0.02135976 |
| GOTERM_CC_ALL | GO: 0005604~basement membrane | 6 | 0.00192342 | 6.56870404 | 0.02164046 |
| GOTERM_MF_ALL | GO: 0004930~G-protein coupled receptor activity | 13 | 0.00026195 | 3.57735882 | 0.02172968 |
| GOTERM_BP_ALL | GO: 0045597~positive regulation of cell differentiation | 13 | 0.00125057 | 3.00345662 | 0.02397951 |
| GOTERM_MF_ALL | GO: 0004896~cytokine receptor activity | 7 | 0.00025702 | 7.58468866 | 0.02483432 |
| GOTERM_BP_ALL | GO: 0051249~regulation of lymphocyte activation | 10 | 0.00150309 | 3.67089142 | 0.02825331 |
| GOTERM_BP_ALL | GO: 0007179~transforming growth factor beta receptor signaling | 7 | 0.00149516 | 5.50633714 | 0.0283535 |
| | pathway | | | | |
| GOTERM_BP_ALL | GO: 0043627~response to estrogen stimulus | 8 | 0.00152438 | 4.63691548 | 0.02840023 |
| GOTERM_BP_ALL | GO: 0009888~tissue development | 21 | 0.00154092 | 2.17491686 | 0.02845818 |
| GOTERM_MF_ALL | GO: 0001871~pattern binding | 9 | 0.00041201 | 4.95326606 | 0.02978498 |
| GOTERM_MF_ALL | GO: 0030247~polysaccharide binding | 9 | 0.00041201 | 4.95326606 | 0.02978498 |
| GOTERM_BP_ALL | GO: 0006869~lipid transport | 10 | 0.00175493 | 3.59108944 | 0.03207509 |
| GOTERM_BP_ALL | GO: 0050818~regulation of coagulation | 5 | 0.00177021 | 9.17722856 | 0.03207792 |
| GOTERM_BP_ALL | GO: 0032946~positive regulation of mononuclear cell proliferation | 6 | 0.00184227 | 6.60760456 | 0.03308502 |
| GOTERM_BP_ALL | GO: 0070665~positive regulation of leukocyte proliferation | 6 | 0.00184227 | 6.60760456 | 0.03308502 |
| GOTERM_BP_ALL | GO: 0050671~positive regulation of lymphocyte proliferation | 6 | 0.00184227 | 6.60760456 | 0.03308502 |
| GOTERM_BP_ALL | GO: 0002526~acute inflammatory response | 7 | 0.00191336 | 5.25604908 | 0.03405697 |
| GOTERM_BP_ALL | GO: 0043066~negative regulation of apoptosis | 18 | 0.0020912 | 2.29607881 | 0.0365633 |
| GOTERM_BP_ALL | GO: 0042592~homeostatic process | 25 | 0.00207425 | 1.95260182 | 0.03656882 |
| GOTERM_BP_ALL | GO: 0007204~elevation of cytosolic calcium ion concentration | 7 | 0.00215298 | 5.13924799 | 0.03732145 |
| KEGG_PATHWAY | hsa04610: Complement and coagulation cascades | 6 | 0.00112812 | 7.15463138 | 0.0376508 |
| GOTERM_BP_ALL | GO: 0009617~response to bacterium | 10 | 0.00219498 | 3.47768661 | 0.03773294 |
| GOTERM_BP_ALL | GO: 0043069~negative regulation of programmed cell death | 18 | 0.00237947 | 2.26978783 | 0.04051986 |
| GOTERM_BP_ALL | GO: 0009725~response to hormone stimulus | 16 | 0.00241757 | 2.42480901 | 0.04083361 |
| GOTERM_BP_ALL | GO: 0031328~positive regulation of cellular biosynthetic process | 25 | 0.00244022 | 1.92979105 | 0.04088828 |
| GOTERM_BP_ALL | GO: 0060548~negative regulation of cell death | 18 | 0.00248193 | 2.26115745 | 0.04125275 |
| GOTERM_MF_ALL | GO: 0030246~carbohydrate binding | 14 | 0.00073273 | 3.01503152 | 0.0421143 |
| GOTERM_CC_ALL | GO: 0000267~cell fraction | 32 | 0.00404178 | 1.69344233 | 0.04306656 |
| GOTERM_MF_ALL | GO: 0019899~enzyme binding | 25 | 0.0008491 | 2.08872665 | 0.04431816 |
| GOTERM_MF_ALL | GO: 0001608~nucleotide receptor activity, G-protein coupled | 5 | 0.00070835 | 11.5576208 | 0.04516455 |
| GOTERM_MF_ALL | GO: 0045028~purinergic nucleotide receptor activity, G-protein coupled | 5 | 0.00070835 | 11.5576208 | 0.04516455 |
| GOTERM_BP_ALL | GO: 0050801~ion homeostasis | 15 | 0.00286437 | 2.47785171 | 0.04710319 |
| GOTERM_BP_ALL | GO: 0003008~system process | 26 | 0.00293122 | 1.87143484 | 0.04745392 |
| GOTERM_BP_ALL | GO: 0040012~regulation of locomotion | 11 | 0.00291736 | 3.07981569 | 0.04759185 |
| GOTERM_BP_ALL | GO: 0031349~positive regulation of defense response | 7 | 0.00300863 | 4.81804499 | 0.04831554 |
| GOTERM_BP_ALL | GO: 0009891~positive regulation of biosynthetic process | 25 | 0.00310955 | 1.89438204 | 0.04916602 |
| GOTERM_BP_ALL | GO: 0009966~regulation of signal transduction | 31 | 0.00313297 | 1.73884331 | 0.04916714 |
| GOTERM_BP_ALL | GO: 0060341~regulation of cellular localization | 11 | 0.00310259 | 3.05393488 | 0.04942072 |

Among the top probesets downregulated by Copaxone with FC≦1.3, six pathways were enriched. This includes developmental processes (as also seen in prior studies e.g. FDA-2014-P-0933-0001 available at www.regulations.gov/#!documentDetail;D=FDA-2014-P-0933-0001, and also described herein), and cell-cell adhesion related pathways.

The top 25 pathways enriched among top upregulated probesets are shown in Table 25, and FIG. 35 illustrates pathways enriched among top probesets differentially modulated by Copaxone® (|FC|≧1.3, FDR p<0.05). As an example of an enriched pathway anticipated to be relevant to Copaxone® mechanism of action, cytokine-cytokine receptor interaction, the individual genes modulated by Copaxone® involved in this pathway are depicted in FIG. 36.

TABLE 25
Top Pathways enriched among top probesets upregulated by
Copaxone ®

| Category | Term | Count^{a} | Adjusted P value |
|---|---|---|---|
| GO Cellular | GO: 0005886~plasma membrane | 122 | 5.85E−23 |
| Compartment | | | |
| GO Cellular | GO: 0031226~intrinsic to plasma | 58 | 8.04E−17 |
| Compartment | membrane | | |
| GO Biological Process | GO: 0009611~response to wounding | 44 | 2.16E−16 |
| GO Biological Process | GO: 0009605~response to external | 58 | 2.34E−16 |
| | stimulus | | |
| GO Biological Process | GO: 0050896~response to stimulus | 118 | 4.45E−16 |
| GO Cellular | GO: 0005887~integral to plasma | 56 | 6.29E−16 |
| Compartment | membrane | | |
| GO Biological Process | GO: 0006955~immune response | 46 | 4.79E−14 |
| GO Biological Process | GO: 0002376~immune system process | 59 | 5.97E−14 |
| GO Cellular | GO: 0016020~membrane | 174 | 6.33E−13 |
| Compartment | | | |
| GO Cellular | GO: 0005576~extracellular region | 58 | 6.45E−13 |
| Compartment | | | |
| GO Biological Process | GO: 0006952~defense response | 40 | 3.86E−12 |
| GO Cellular | GO: 0044459~plasma membrane part | 76 | 4.00E−12 |
| Compartment | | | |
| GO Molecular Function | GO: 0004888~transmembrane receptor | 39 | 8.19E−12 |
| | activity | | |
| GO Molecular Function | GO: 0060089~molecular transducer | 69 | 1.15E−11 |
| | activity | | |
| GO Molecular Function | GO: 0004871~signal transducer activity | 69 | 1.15E−11 |
| GO Molecular Function | GO: 0004872~receptor activity | 56 | 2.05E−11 |
| GO Cellular | GO: 0044421~extracellular region part | 37 | 5.16E−11 |
| Compartment | | | |
| GO Biological Process | GO: 0006954~inflammatory response | 28 | 3.57E−10 |
| GO Cellular | GO: 0031224~intrinsic to membrane | 125 | 3.08E−09 |
| Compartment | | | |
| GO Cellular | GO: 0016021~integral to membrane | 121 | 8.31E−09 |
| Compartment | | | |
| GO Cellular | GO: 0044425~membrane part | 146 | 3.43E−08 |
| Compartment | | | |
| Kegg Pathway | hsa04060: Cytokine-cytokine receptor | 21 | 5.00E−08 |
| | interaction | | |
| GO Cellular | GO: 0005615~extracellular space | 27 | 5.72E−08 |
| Compartment | | | |
| GO Biological Process | GO: 0006950~response to stress | 70 | 1.44E−07 |
| GO Biological Process | GO: 0042060~wound healing | 19 | 1.90E−07 |

^{a}Count refers to number of genes within the list of interest annotated to the pathway.
GO database: Ashburner, M. et al. Gene ontology: tool for the unification of biology. The Gene Ontology Consortium. Nat. Genet. 25, 25-29 (2000).
Kegg database: Kanehisa, M. & Goto, S. KEGG: kyoto encyclopedia of genes and genomes. Nucleic Acids Res. 28, 27-30 (2000).
Copaxone MoA discussion

The fact that 12,115 probesets are significantly modulated by Copaxone relative to mannitol provides a window into the complexity of Copaxone®'s mechanism of action on antigen-presenting cells. Despite the complexity and scope of this modulation, key genes known to be modulated by Copaxone® (such as IL1RN, IL1ORA) appear in the expected directionality. These modulated genes are significantly enriched in immunology-related pathways which are known to be relevant for Copaxone's mechanism of action. These two aspects combined together validate the design of the study and its ability to identify relevant aspects of a medicine's interaction with antigen-presenting cells.

### Polimunol and Copaxone® Comparison: Differentially Expressed Genes

Comparison of Polimunol versus Copaxone® treatment, corrected for Mannitol control expression [i.e. (Polimunol-Mannitol)-(Copaxone-Mannitol)], resulted in 807 probesets differentially expressed with FDR-adjusted p<0.05. Upon filtering for “present” calls only, 518 upregulated probesets and 289 downregulated probesets were detected.

After conservative filtering out of highly variable probesets (i.e. the few previously identified to have differing behavior between THP-1 studies, which removed 3.5% of the 807 probesets), 779 modulated probesets persist: 494 upregulated probesets and 285 downregulated probesets. After these filtering steps, the top 25 upregulated probesets are shown in Table 26, and the top 25 downregulated probesets are shown in Table 27.

TABLE 26
Probesets significantly upregulated in Polimunol versus
Copaxone ®

| Probeset | Gene | raw.FC | P.Value | adj.P.Value |
|---|---|---|---|---|
| 212665_at | TIPARP | 1.18 | 2.68E−17 | 1.47E−12 |
| 202436_s_at | CYP1B1 | 1.41 | 1.64E−15 | 4.49E−11 |
| 224735_at | CYBASC3 | 1.16 | 3.50E−14 | 6.37E−10 |
| 209921_at | SLC7A11 | 1.35 | 2.57E−13 | 3.51E−09 |
| 202435_s_at | CYP1B1 | 1.37 | 3.31E−13 | 3.62E−09 |
| 208937_s_at | ID1 | 1.22 | 1.03E−12 | 9.43E−09 |
| 202434_s_at | CYP1B1 | 1.42 | 2.03E−12 | 1.58E−08 |
| 228964_at | PRDM1 | 1.24 | 3.29E−12 | 2.07E−08 |
| 223113_at | TMEM138 | 1.11 | 3.40E−12 | 2.07E−08 |
| 230966_at | IL4I1 | 1.33 | 1.60E−11 | 8.74E−08 |
| 202014_at | PPP1R15A | 1.24 | 1.86E−11 | 9.24E−08 |
| 202437_s_at | CYP1B1 | 1.34 | 2.45E−11 | 1.06E−07 |
| 37028_at | PPP1R15A | 1.19 | 2.52E−11 | 1.06E−07 |
| 39402_at | IL1B | 1.23 | 3.09E−11 | 1.21E−07 |
| 205067_at | IL1B | 1.28 | 4.02E−11 | 1.47E−07 |
| 217678_at | SLC7A11 | 1.29 | 6.91E−11 | 2.36E−07 |
| 216598_s_at | CCL2 | 1.35 | 8.57E−11 | 2.67E−07 |
| 203821_at | HBEGF | 1.24 | 8.80E−11 | 2.67E−07 |
| 201468_s_at | NQO1 | 1.16 | 9.34E−11 | 2.69E−07 |
| 225252_at | SRXN1 | 1.12 | 1.12E−10 | 3.07E−07 |
| 211307_s_at | FCAR | 1.27 | 1.31E−10 | 3.41E−07 |
| 209928_s_at | MSC | 1.19 | 1.40E−10 | 3.48E−07 |
| 201266_at | TXNRD1 | 1.07 | 2.22E−10 | 5.28E−07 |
| 210519_s_at | NQO1 | 1.14 | 3.76E−10 | 8.22E−07 |
| 212171_x_at | VEGF | 1.14 | 1.22E−09 | 2.46E−06 |

TABLE 27
Probesets significantly downregulated in Polimunol versus
Copaxone ®

| Probeset | Gene | raw.FC | P.Value | adj.P.Val. |
|---|---|---|---|---|
| 204614_at | SERPINB2 | −1.16 | 4.60E−10 | 9.68E−07 |
| 211421_s_at | RET | −1.13 | 1.75E−09 | 3.41E−06 |
| 207725_at | POU4F2 | −1.16 | 3.49E−09 | 6.15E−06 |
| 208206_s_at | RASGRP2 | −1.11 | 1.56E−08 | 1.82E−05 |
| 218971_s_at | HSPC049 | −1.15 | 3.32E−08 | 3.04E−05 |
| 222799_at | HSPC049 | −1.17 | 5.13E−08 | 4.25E−05 |
| 219463_at | C20ORF103 | −1.11 | 6.49E−08 | 5.22E−05 |
| 218858_at | DEPDC6 | −1.17 | 8.64E−08 | 6.72E−05 |
| 214539_at | SERPINB10 | −1.14 | 1.06E−07 | 7.91E−05 |
| 225510_at | OAF | −1.08 | 1.88E−07 | 0.000121086 |
| 228855_at | LOC645919 | −1.13 | 2.00E−07 | 0.000125905 |
| 200799_at | HSPA1A | −1.09 | 2.92E−07 | 0.000168544 |
| 206643_at | HAL | −1.11 | 3.17E−07 | 0.000176999 |
| 44790_s_at | C13ORF18 | −1.11 | 3.23E−07 | 0.000178591 |
| 202441_at | SPFH1 | −1.09 | 5.23E−07 | 0.000255211 |
| 206034_at | SERPINB8 | −1.08 | 7.06E−07 | 0.000321557 |
| 203685_at | BCL2 | −1.16 | 7.53E−07 | 0.000340412 |
| 216733_s_at | GATM | −1.09 | 8.49E−07 | 0.000374363 |
| 224428_s_at | CDCA7 | −1.07 | 8.91E−07 | 0.000386704 |
| 201310_s_at | C5ORF13 | −1.11 | 1.13E−06 | 0.000455961 |
| 213361_at | TDRD7 | −1.16 | 1.28E−06 | 0.000504959 |
| 219714_s_at | CACNA2D3 | −1.09 | 1.40E−06 | 0.000542581 |
| 35974_at | LRMP | −1.12 | 1.69E−06 | 0.000628259 |
| 201309_x_at | C5ORF13 | −1.12 | 1.96E−06 | 0.000705871 |
| 243824_at | — | −1.18 | 2.13E−06 | 0.000730854 |

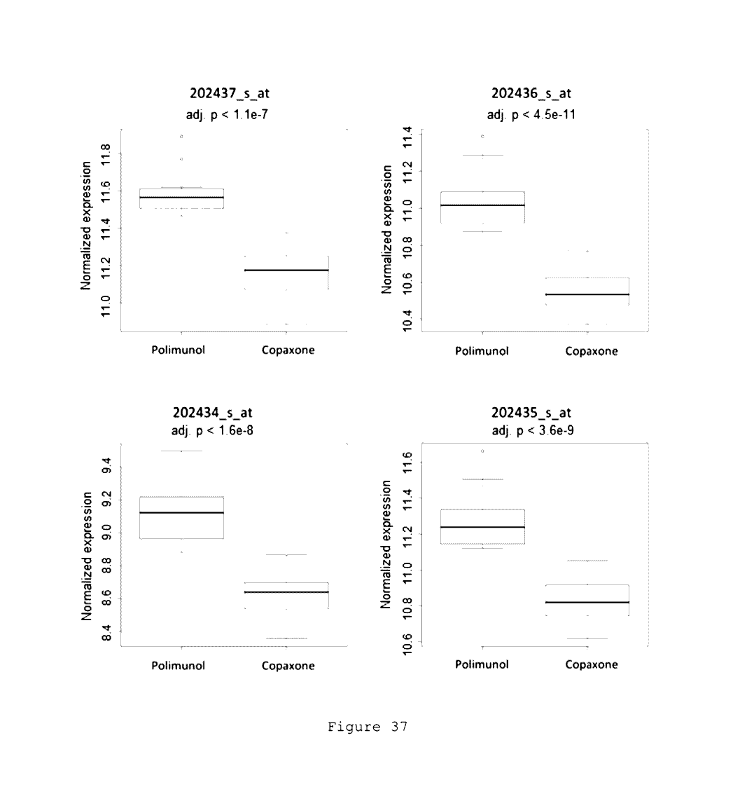

As observed in the earlier study of the same design (FDA-2014-P-0933-0001 available at www.regulations.gov/#?documentDetail;D=FDA-2014-P-0933-0001, and also described herein), Polimunol consistently upregulated CYP1B1 relative to Copaxone® (all four probesets on chip: FDR p values 4.5e-11, 3.6e-9, 1.6e-8, 1.1e-7, See FIG. 37).

However, ADRB2 was not significantly downregulated by Polimunol relative to Copaxone® in the second set of studies (FC −1.06, nominal p 0.06).

The probesets significantly differentially expressed between Polimunol and Copaxone® treatment (corrected for mannitol control) in THP-1 cells is provided in Table 28.

TABLE 28
Probesets significantly differentially expressed between Polimunol and Copaxone ® treatment
(corrected for mannitol control) in THP-1 cells.

| Probeset | Gene | raw. FC | AveExpr | t | P. Value | adj. P. Val | B |
|---|---|---|---|---|---|---|---|
| 212665_at | TIPARP | 1.18491814 | 9.07215527 | 11.883114 | 2.68E−17 | 1.47E−12 | 27.6284062 |
| 202436_s_at | CYP1B1 | 1.40629663 | 10.5046627 | 10.7394789 | 1.64E−15 | 4.49E−11 | 23.9781074 |
| 224735_at | CYBASC3 | 1.16280829 | 11.1371084 | 9.91592252 | 3.50E−14 | 6.37E−10 | 21.2319004 |
| 209921_at | SLC7A11 | 1.34875797 | 8.01938606 | 9.38858221 | 2.57E−13 | 3.51E−09 | 19.4266298 |
| 202435_s_at | CYP1B1 | 1.36824479 | 10.7674593 | 9.32190062 | 3.31E−13 | 3.62E−09 | 19.1959747 |
| 208937_s_at | ID1 | 1.21578992 | 9.79600279 | 9.02417027 | 1.03E−12 | 9.43E−09 | 18.1600689 |
| 202434_s_at | CYP1B1 | 1.42360365 | 8.59154176 | 8.84918014 | 2.03E−12 | 1.58E−08 | 17.5468656 |
| 228964_at | PRDM1 | 1.24239601 | 8.33311381 | 8.72349013 | 3.29E−12 | 2.07E−08 | 17.1045709 |
| 223113_at | TMEM138 | 1.1063561 | 11.1028453 | 8.71472552 | 3.40E−12 | 2.07E−08 | 17.0736735 |
| 230966_at | IL4I1 | 1.32516688 | 8.36704038 | 8.31508719 | 1.60E−11 | 8.74E−08 | 15.657823 |
| 202014_at | PPP1R15A | 1.23664454 | 8.57467088 | 8.27614067 | 1.86E−11 | 9.24E−08 | 15.5191648 |
| 202437_s_at | CYP1B1 | 1.34228274 | 11.0857103 | 8.20543116 | 2.45E−11 | 1.06E−07 | 15.2671479 |
| 37028_at | PPP1R15A | 1.18998338 | 9.10953004 | 8.19801576 | 2.52E−11 | 1.06E−07 | 15.2406986 |
| 39402_at | IL1B | 1.22916144 | 8.58599227 | 8.14511174 | 3.09E−11 | 1.21E−07 | 15.0518933 |
| 205067_at | IL1B | 1.27678411 | 8.78154185 | 8.0777594 | 4.02E−11 | 1.47E−07 | 14.8112634 |
| 217678_at | SLC7A11 | 1.28685305 | 7.69726333 | 7.93874264 | 6.91E−11 | 2.36E−07 | 14.3137409 |
| 216598_s_at | CCL2 | 1.34792469 | 9.28596162 | 7.88363055 | 8.57E−11 | 2.67E−07 | 14.116209 |
| 203821_at | HBEGF | 1.23590003 | 9.26713046 | 7.87666898 | 8.80E−11 | 2.67E−07 | 14.0912465 |
| 201468_s_at | NQO1 | 1.15576656 | 9.35668628 | 7.86152927 | 9.34E−11 | 2.69E−07 | 14.0369508 |
| 225252_at | SRXN1 | 1.11737221 | 9.70265778 | 7.81446478 | 1.12E−10 | 3.07E−07 | 13.8680929 |
| 211307_s_at | FCAR | 1.27074012 | 7.1312048 | 7.77503632 | 1.31E−10 | 3.41E−07 | 13.7265534 |
| 209928_s_at | MSC | 1.18769631 | 8.08116641 | 7.75739241 | 1.40E−10 | 3.48E−07 | 13.6631936 |
| 201266_at | TXNRD1 | 1.06947321 | 11.9113637 | 7.6396293 | 2.22E−10 | 5.28E−07 | 13.239984 |
| 210519_s_at | NQO1 | 1.13794807 | 10.6816859 | 7.50492043 | 3.76E−10 | 8.22E−07 | 12.7552954 |
| 212171_x_at | VEGF | 1.13674051 | 9.43661194 | 7.20421256 | 1.22E−09 | 2.46E−06 | 11.6719358 |
| 38037_at | HBEGF | 1.18482724 | 7.98711019 | 7.0523034 | 2.20E−09 | 4.14E−06 | 11.1244167 |
| 201118_at | PGD | 1.07758185 | 12.4845488 | 6.97857101 | 2.93E−09 | 5.34E−06 | 10.8587242 |
| 203045_at | NINJ1 | 1.12384597 | 9.41715175 | 6.88585707 | 4.21E−09 | 7.19E−06 | 10.5247541 |
| 216250_s_at | LPXN | 1.24070246 | 10.2461732 | 6.84611362 | 4.91E−09 | 7.90E−06 | 10.3816492 |
| 210512_s_at | VEGF | 1.17453419 | 10.5171208 | 6.84606411 | 4.91E−09 | 7.90E−06 | 10.3814709 |
| 210592_s_at | SAT1 | 1.14616249 | 9.85711211 | 6.78833303 | 6.15E−09 | 9.16E−06 | 10.1736745 |
| 210095_s_at | IGFBP3 | 1.26126777 | 7.77688369 | 6.7861051 | 6.20E−09 | 9.16E−06 | 10.1656573 |
| 208893_s_at | DUSP6 | 1.23681965 | 10.7702231 | 6.7782857 | 6.39E−09 | 9.20E−06 | 10.1375206 |
| 202284_s_at | CDKN1A | 1.12078798 | 10.6198443 | 6.75691033 | 6.95E−09 | 9.69E−06 | 10.0606157 |
| 209122_at | ADFP | 1.14098228 | 12.3228398 | 6.75171809 | 7.09E−09 | 9.69E−06 | 10.0419373 |
| 228937_at | C13ORF31 | 1.17921905 | 8.37907051 | 6.70099976 | 8.63E−09 | 1.15E−05 | 9.8595365 |
| 201235_s_at | BTG2 | 1.28386355 | 7.44589095 | 6.64539327 | 1.07E−08 | 1.39E−05 | 9.65967492 |
| 203927_at | NFKBIE | 1.12895866 | 9.72640239 | 6.63162768 | 1.13E−08 | 1.44E−05 | 9.61021946 |
| 1553141_at | C13ORF31 | 1.19000046 | 6.93831195 | 6.55368898 | 1.53E−08 | 1.82E−05 | 9.33038387 |
| 220266_s_at | KLF4 | 1.1368523 | 8.46464122 | 6.55037647 | 1.55E−08 | 1.82E−05 | 9.31849743 |
| 217995_at | SQRDL | 1.0793508 | 12.336991 | 6.54729812 | 1.57E−08 | 1.82E−05 | 9.30745172 |
| 207674_at | FCAR | 1.24494467 | 7.73429071 | 6.53420308 | 1.65E−08 | 1.88E−05 | 9.26047019 |
| 46665_at | SEMA4C | 1.10637799 | 10.5657844 | 6.4481698 | 2.30E−08 | 2.53E−05 | 8.95205496 |
| 210264_at | GPR35 | 1.11391738 | 8.72115064 | 6.44628003 | 2.32E−08 | 2.53E−05 | 8.94528559 |
| 211668_s_at | PLAU | 1.11120293 | 9.2353654 | 6.42233001 | 2.54E−08 | 2.69E−05 | 8.85951421 |
| 205205_at | RELB | 1.16492706 | 7.34250559 | 6.42094346 | 2.56E−08 | 2.69E−05 | 8.85454977 |
| 203798_s_at | VSNL1 | 1.16614475 | 7.2833092 | 6.41517707 | 2.61E−08 | 2.70E−05 | 8.83390503 |
| 219634_at | CHST11 | 1.11031386 | 9.57653235 | 6.40091795 | 2.76E−08 | 2.75E−05 | 8.78286459 |
| 201502_s_at | NFKBIA | 1.14603586 | 11.2391775 | 6.40005102 | 2.77E−08 | 2.75E−05 | 8.77976186 |
| 229055_at | GPR68 | 1.14767352 | 7.68909756 | 6.36111525 | 3.22E−08 | 3.04E−05 | 8.64046681 |
| 212190_at | SERPINE2 | 1.12333317 | 9.662056 | 6.3519722 | 3.34E−08 | 3.04E−05 | 8.60777296 |
| 219386_s_at | SLAMF8 | 1.34835612 | 8.40890612 | 6.31827296 | 3.80E−08 | 3.41E−05 | 8.48732535 |
| 210513_s_at | VEGF | 1.15191285 | 8.36243556 | 6.28246816 | 4.36E−08 | 3.85E−05 | 8.35944956 |
| 211527_x_at | VEGF | 1.13731715 | 7.87125822 | 6.26106285 | 4.74E−08 | 4.11E−05 | 8.28305106 |
| 1555638_a_a | SAMSN1 | 1.16504788 | 8.69549173 | 6.24146956 | 5.11E−08 | 4.25E−05 | 8.21315379 |
| 204470_at | CXCL1 | 1.35501196 | 7.67502936 | 6.24129687 | 5.11E−08 | 4.25E−05 | 8.2125379 |
| 220935_s_at | CDK5RAP2 | 1.10875555 | 9.47138345 | 6.11902224 | 8.18E−08 | 6.48E−05 | 7.7771196 |
| 201236_s_at | BTG2 | 1.39106574 | 9.51819834 | 6.10195957 | 8.73E−08 | 6.72E−05 | 7.71647403 |
| 228648_at | LRG1 | 1.13667657 | 10.1778893 | 6.09346129 | 9.02E−08 | 6.85E−05 | 7.68627977 |
| 212143_s_at | IGFBP3 | 1.19756801 | 7.50292055 | 6.0375024 | 1.12E−07 | 8.26E−05 | 7.48764791 |
| 240076_at | — | 1.11560154 | 7.77294938 | 6.02250815 | 1.18E−07 | 8.63E−05 | 7.43448169 |
| 215223_s_at | SOD2 | 1.08832613 | 9.38708957 | 6.00345187 | 1.27E−07 | 9.16E−05 | 7.36694855 |
| 208891_at | DUSP6 | 1.20973898 | 11.4952332 | 5.99682168 | 1.31E−07 | 9.18E−05 | 7.34346158 |
| 228967_at | EIF1 | 1.09256705 | 7.51282579 | 5.99430219 | 1.32E−07 | 9.18E−05 | 7.33453779 |
| 201489_at | PPIF | 1.07056711 | 11.9745223 | 5.98944612 | 1.34E−07 | 9.18E−05 | 7.31734011 |
| 202388_at | RGS2 | 1.26773664 | 9.57646356 | 5.97863153 | 1.40E−07 | 9.45E−05 | 7.27905017 |
| 221903_s_at | CYLD | 1.09642353 | 9.26863731 | 5.97193254 | 1.44E−07 | 9.58E−05 | 7.25533872 |
| 1560228_at | SNAI3 | 1.15060555 | 7.09991102 | 5.93825721 | 1.63E−07 | 0.0001076 | 7.13622321 |
| 227034_at | ANKRD57 | 1.22029842 | 7.32299473 | 5.88467981 | 2.00E−07 | 0.0001259 | 6.94699581 |
| 200878_at | EPAS1 | 1.12190973 | 8.895976 | 5.87823307 | 2.05E−07 | 0.00012757 | 6.9242512 |
| 201005_at | CD9 | 1.14821208 | 9.10086802 | 5.86983584 | 2.12E−07 | 0.00013023 | 6.89463315 |
| 206026_s_at | TNFAIP6 | 1.19240437 | 5.36372703 | 5.85839211 | 2.21E−07 | 0.00013373 | 6.85428439 |
| 214290_s_at | HIST2H2AA / | 1.09794003 | 11.8199009 | 5.85526922 | 2.24E−07 | 0.00013373 | 6.84327656 |
| 222670_s_at | MAFB | 1.20369944 | 9.73888237 | 5.854147 | 2.25E−07 | 0.00013373 | 6.83932116 |
| 205153_s_at | CD40 | 1.13334093 | 8.72577521 | 5.82226469 | 2.54E−07 | 0.00014934 | 6.72701829 |
| 225390_s_at | KLF13 | 1.08054083 | 10.877664 | 5.78479854 | 2.93E−07 | 0.00016854 | 6.59522285 |
| 227080_at | ZNF697 | 1.12343463 | 8.92012752 | 5.76864695 | 3.11E−07 | 0.000177 | 6.5384663 |
| 214056_at | MCL1 | 1.09148142 | 8.9472801 | 5.76468003 | 3.16E−07 | 0.000177 | 6.52453225 |
| 201739_at | SGK | 1.28779242 | 8.00577284 | 5.75044243 | 3.34E−07 | 0.00018189 | 6.47454023 |
| 201170_s_at | BHLHB2 | 1.09531573 | 9.50744497 | 5.74853964 | 3.36E−07 | 0.00018189 | 6.46786122 |
| 205633_s_at | ALAS1 | 1.08179152 | 11.5068057 | 5.74324623 | 3.43E−07 | 0.00018376 | 6.44928355 |
| 203665_at | HMOX1 | 1.24623576 | 9.15770135 | 5.7289493 | 3.62E−07 | 0.00019209 | 6.39912762 |
| 203936_s_at | MMP9 | 1.145748 | 8.56115455 | 5.70212398 | 4.01E−07 | 0.0002066 | 6.30510148 |
| 213988_s_at | SAT1 | 1.12993548 | 9.10818688 | 5.68515244 | 4.27E−07 | 0.00021822 | 6.24566986 |
| 225097_at | HIPK2 | 1.16355832 | 9.37790876 | 5.66703291 | 4.57E−07 | 0.00023152 | 6.18226665 |
| 226218_at | — | 1.2939063 | 6.28386762 | 5.66047476 | 4.69E−07 | 0.00023514 | 6.15933112 |
| 203939_at | NT5E | 1.19431475 | 7.2701384 | 5.6470707 | 4.93E−07 | 0.00024509 | 6.11247457 |
| 221840_at | PTPRE | 1.10358333 | 8.99406461 | 5.64033312 | 5.06E−07 | 0.00024914 | 6.0889327 |
| 234994_at | KIAA1913 | 1.26172422 | 6.19165569 | 5.61287248 | 5.61E−07 | 0.00026903 | 5.99305687 |
| 201169_s_at | BHLHB2 | 1.1138348 | 7.81678741 | 5.60701185 | 5.73E−07 | 0.00027264 | 5.97261084 |
| 223222_at | SLC25A19 | 1.06195494 | 11.1387437 | 5.60415209 | 5.80E−07 | 0.00027322 | 5.96263602 |
| 201250_s_at | SLC2A1 | 1.07198576 | 9.45224146 | 5.58245393 | 6.29E−07 | 0.00029287 | 5.88699657 |
| 205770_at | GSR | 1.08507865 | 10.4221632 | 5.58115446 | 6.32E−07 | 0.00029287 | 5.88246909 |
| 211610_at | KLF6 | 1.21373848 | 7.58295098 | 5.56973288 | 6.60E−07 | 0.00030315 | 5.84268743 |
| 217996_at | PHLDA1 | 1.33234556 | 7.22644861 | 5.52201205 | 7.89E−07 | 0.00035373 | 5.6767135 |
| 204440_at | CD83 | 1.09559915 | 9.65465598 | 5.51721887 | 8.04E−07 | 0.00035722 | 5.66006438 |
| 203751_x_at | JUND | 1.11709364 | 8.15242666 | 5.49344944 | 8.78E−07 | 0.00038425 | 5.57756091 |
| 220330_s_at | SAMSN1 | 1.16271877 | 9.59945919 | 5.48604123 | 9.03E−07 | 0.00038884 | 5.55186756 |
| 202644_s_at | TNFAIP3 | 1.10355352 | 10.1094931 | 5.47992259 | 9.24E−07 | 0.00039474 | 5.53065412 |
| 203797_at | VSNL1 | 1.10477681 | 8.15296158 | 5.47701685 | 9.34E−07 | 0.00039597 | 5.52058224 |
| 201069_at | MMP2 | 1.10375779 | 8.40697176 | 5.45565127 | 1.01E−06 | 0.00042561 | 5.44657164 |
| 204653_at | TFAP2A | 1.17142932 | 7.72005745 | 5.44704422 | 1.05E−06 | 0.00043617 | 5.41678026 |
| 219385_at | SLAMF8 | 1.21995297 | 7.92135191 | 5.44342079 | 1.06E−06 | 0.00043648 | 5.40424263 |
| 208926_at | NEU1 | 1.08099136 | 10.7507527 | 5.44280336 | 1.06E−06 | 0.00043648 | 5.40210647 |
| 239412_at | IRF5 | 1.07460841 | 10.9779751 | 5.42713112 | 1.13E−06 | 0.00045596 | 5.34790799 |
| 223501_at | — | 1.1103323 | 9.76378918 | 5.42513574 | 1.13E−06 | 0.00045596 | 5.34101078 |
| 201651_s_at | PACSIN2 | 1.05371079 | 12.4689697 | 5.41770064 | 1.17E−06 | 0.00046511 | 5.31531719 |
| 214211_at | FTH1 | 1.15283111 | 10.7669406 | 5.41590299 | 1.17E−06 | 0.00046511 | 5.30910659 |
| 202988_s_at | RGS1 | 1.26703574 | 6.64018217 | 5.37761912 | 1.35E−06 | 0.00052878 | 5.17698759 |
| 223303_at | URP2 | 1.07136267 | 11.4584296 | 5.36688864 | 1.41E−06 | 0.00054258 | 5.14000695 |
| 36711_at | MAFF | 1.19353834 | 6.77574709 | 5.36039903 | 1.44E−06 | 0.00055196 | 5.11765264 |
| 203508_at | TNFRSF1B | 1.11118203 | 9.41800725 | 5.35852346 | 1.45E−06 | 0.00055196 | 5.1111935 |
| 227645_at | PIK3R5 | 1.10436883 | 8.17453287 | 5.33951385 | 1.56E−06 | 0.0005883 | 5.04576711 |
| 218501_at | ARHGEF3 | 1.11215762 | 8.30700755 | 5.31477003 | 1.71E−06 | 0.00063184 | 4.96071299 |
| 209606_at | PSCDBP | 1.18667973 | 7.12974957 | 5.30711238 | 1.76E−06 | 0.00064569 | 4.93441572 |
| 1555756_a_a | CLEC7A | 1.21395231 | 6.44819327 | 5.2975806 | 1.82E−06 | 0.00066446 | 4.90169919 |
| 229354_at | PDCD6 /// A [symbol] | 1.08957565 | 10.3878046 | 5.29027036 | 1.87E−06 | 0.00067819 | 4.87662042 |
| 233955_x_at | CXXC5 | 1.07971504 | 12.6052495 | 5.27206493 | 2.00E−06 | 0.00071277 | 4.81421221 |
| 203887_s_at | THBD | 1.14864911 | 11.9807825 | 5.27153474 | 2.01E−06 | 0.00071277 | 4.81239575 |
| 233540_s_at | CDK5RAP2 | 1.08483341 | 10.2227011 | 5.26920484 | 2.03E−06 | 0.00071431 | 4.80441408 |
| 209774_x_at | CXCL2 | 1.33797035 | 6.42227624 | 5.26653762 | 2.05E−06 | 0.00071677 | 4.79527824 |
| 210845_s_at | PLAUR | 1.12269107 | 10.7660847 | 5.26173913 | 2.08E−06 | 0.0007209 | 4.77884607 |
| 225980_at | C14ORF43 | 1.0793459 | 9.21104798 | 5.23040107 | 2.34E−06 | 0.00079874 | 4.67165036 |
| 219039_at | SEMA4C | 1.11139871 | 9.52722178 | 5.2215591 | 2.41E−06 | 0.0008154 | 4.64144331 |
| 228754_at | SLC6A6 | 1.11066199 | 9.56424888 | 5.21977863 | 2.43E−06 | 0.0008154 | 4.63536267 |
| 222062_at | IL27RA | 1.14734393 | 8.84896859 | 5.21600492 | 2.47E−06 | 0.0008189 | 4.62247702 |
| 208892_s_at | DUSP6 | 1.19436158 | 11.0841436 | 5.21531302 | 2.47E−06 | 0.0008189 | 4.62011481 |
| 204684_at | NPTX1 | 1.28533088 | 9.73612701 | 5.20831506 | 2.54E−06 | 0.00083526 | 4.59622895 |
| 208373_s_at | P2RY6 | 1.06697743 | 9.65819159 | 5.20192905 | 2.60E−06 | 0.00084937 | 4.57444113 |
| 218856_at | TNFRSF21 | 1.11783326 | 11.1389024 | 5.20052327 | 2.61E−06 | 0.00084937 | 4.56964607 |
| 204436_at | PLEKHQ1 | 1.09698664 | 9.3889143 | 5.19357309 | 2.68E−06 | 0.0008652 | 4.54594574 |
| 212227_x_at | EIF1 | 1.05443994 | 12.7811739 | 5.19230534 | 2.69E−06 | 0.0008652 | 4.54162382 |
| 223125_s_at | C1ORF21 | 1.11079328 | 10.4937493 | 5.18218325 | 2.79E−06 | 0.00089281 | 4.50712903 |
| 217997_at | PHLDA1 | 1.26744571 | 6.13655327 | 5.17537259 | 2.86E−06 | 0.0009049 | 4.48393197 |
| 204912_at | IL10RA | 1.12420434 | 9.40611087 | 5.17279988 | 2.89E−06 | 0.00090826 | 4.47517201 |
| 218417_s_at | FLJ20489 | 1.08815767 | 9.3505492 | 5.15361341 | 3.10E−06 | 0.0009514 | 4.4098898 |
| 203888_at | THBD | 1.15159161 | 10.9696824 | 5.15288486 | 3.11E−06 | 0.0009514 | 4.40741251 |
| 205419_at | EBI2 | 1.18215692 | 9.40684523 | 5.1527513 | 3.11E−06 | 0.0009514 | 4.4069584 |
| 228708_at | RAB27B | 1.18815027 | 7.90684935 | 5.15257387 | 3.11E−06 | 0.0009514 | 4.40635511 |
| 200644_at | MARCKSL1 | 1.10690452 | 11.028723 | 5.15248432 | 3.11E−06 | 0.0009514 | 4.40605064 |
| 202021_x_at | EIF1 | 1.05923104 | 12.6600822 | 5.1394264 | 3.27E−06 | 0.00099262 | 4.36167177 |
| 211653_x_at | AKR1C2 | 1.12714868 | 8.13501584 | 5.12100436 | 3.50E−06 | 0.00104753 | 4.29912857 |
| 209882_at | RIT1 | 1.10219332 | 10.0435725 | 5.12025544 | 3.51E−06 | 0.00104753 | 4.29658764 |
| 201467_s_at | NQO1 | 1.11423301 | 9.57842418 | 5.10680582 | 3.68E−06 | 0.00109451 | 4.25097768 |
| 225842_at | PHLDA1 | 1.16531676 | 7.08579562 | 5.09284521 | 3.88E−06 | 0.00114573 | 4.20367946 |
| 220091_at | SLC2A6 | 1.11656636 | 9.02115478 | 5.08631413 | 3.97E−06 | 0.00116392 | 4.18156801 |
| 200920_s_at | BTG1 | 1.06979743 | 10.8993308 | 5.08304104 | 4.02E−06 | 0.00116392 | 4.17049051 |
| 222703_s_at | YRDC | 1.06438343 | 10.3475422 | 5.08288187 | 4.02E−06 | 0.00116392 | 4.1699519 |
| 201490_s_at | PPIF | 1.05947022 | 11.5950549 | 5.07132874 | 4.19E−06 | 0.00120065 | 4.13087206 |
| 218223_s_at | PLEKHO1 | 1.12093336 | 9.10575582 | 5.06452081 | 4.30E−06 | 0.00122451 | 4.10785829 |
| 201851_at | SH3GL1 | 1.07901872 | 9.65074195 | 5.02668705 | 4.94E−06 | 0.00138427 | 3.98016723 |
| 228124_at | ABHD12 | 1.09977731 | 7.66951417 | 5.01836495 | 5.09E−06 | 0.00141465 | 3.95212649 |
| 218559_s_at | MAFB | 1.22135659 | 10.2200187 | 5.01793255 | 5.10E−06 | 0.00141465 | 3.95066998 |
| 228696_at | SLC45A3 | 1.12825933 | 7.72464342 | 5.01306718 | 5.19E−06 | 0.00143268 | 3.93428488 |
| 223660_at | ADORA3 | 1.13136045 | 7.77795316 | 4.98586352 | 5.73E−06 | 0.00155048 | 3.84277934 |
| 220066_at | CARD15 | 1.12730802 | 7.8810279 | 4.98180156 | 5.81E−06 | 0.0015658 | 3.82913191 |
| 238542_at | ULBP2 | 1.10589961 | 7.94992924 | 4.9769201 | 5.92E−06 | 0.00158601 | 3.81273664 |
| 224480_s_at | LPAAT-THET [symbol] | 1.20881651 | 8.02630766 | 4.96873318 | 6.10E−06 | 0.00162593 | 3.78525289 |
| 207469_s_at | PIR | 1.10472357 | 9.02435046 | 4.96686989 | 6.14E−06 | 0.00162902 | 3.77900012 |
| 202241_at | TRIB1 | 1.67898747 | 9.30574701 | 4.94914352 | 6.55E−06 | 0.00172797 | 3.71955903 |
| 204975_at | EMP2 | 1.07979325 | 8.71044082 | 4.94102597 | 6.74E−06 | 0.00176349 | 3.69236569 |
| 226354_at | LACTB | 1.06792187 | 9.9828876 | 4.93326158 | 6.93E−06 | 0.00179962 | 3.66637136 |
| 226022_at | SASH1 | 1.07960243 | 11.2682799 | 4.93280332 | 6.95E−06 | 0.00179962 | 3.66483764 |
| 228869_at | SLIC1 | 1.07164715 | 9.82112889 | 4.92528486 | 7.14E−06 | 0.00184057 | 3.63968244 |
| 203752_s_at | JUND | 1.08201127 | 11.8110521 | 4.92340249 | 7.19E−06 | 0.00184446 | 3.63338672 |
| 212130_x_at | EIF1 | 1.05588847 | 12.6471904 | 4.91712987 | 7.35E−06 | 0.00187608 | 3.6124142 |
| 219593_at | SLC15A3 | 1.08615216 | 8.63335571 | 4.91416769 | 7.43E−06 | 0.00187608 | 3.60251376 |
| 236570_at | ZNF366 | 1.23552697 | 5.63015267 | 4.91397465 | 7.43E−06 | 0.00187608 | 3.60186863 |
| 213236_at | SASH1 | 1.09881243 | 10.3511094 | 4.91356632 | 7.45E−06 | 0.00187608 | 3.60050408 |
| 225609_at | GSR | 1.08973861 | 9.95822522 | 4.90544456 | 7.67E−06 | 0.00192315 | 3.57337186 |
| 205891_at | ADORA2B | 1.11169386 | 9.89367673 | 4.87112294 | 8.68E−06 | 0.00216282 | 3.45890752 |
| 228438_at | TRPA1 | 1.12226677 | 8.86862388 | 4.87039478 | 8.70E−06 | 0.00216282 | 3.45648245 |
| 226372_at | CHST11 | 1.08446345 | 11.0281604 | 4.8686604 | 8.76E−06 | 0.00216654 | 3.45070693 |
| 204961_s_at | NCF1 /// LO [symbol] | 1.09199637 | 10.3669364 | 4.86594187 | 8.84E−06 | 0.00217803 | 3.44165575 |
| 212124_at | RAI17 | 1.09160138 | 9.61628437 | 4.84935418 | 9.39E−06 | 0.00230182 | 3.38647153 |
| 204341_at | TRIM16 /// T [symbol] | 1.10230923 | 8.04807245 | 4.83779913 | 9.79E−06 | 0.0023573 | 3.34807425 |
| 233857_s_at | ASB2 | 1.10322519 | 6.77376513 | 4.82807168 | 1.01E−05 | 0.00243053 | 3.31577846 |
| 205479_s_at | PLAU | 1.08239048 | 9.99849286 | 4.82413541 | 1.03E−05 | 0.00244373 | 3.30271717 |
| 202252_at | RAB13 | 1.04918345 | 12.5262785 | 4.81495419 | 1.06E−05 | 0.00251476 | 3.27226887 |
| 204363_at | F3 | 1.13756401 | 8.52817386 | 4.80950401 | 1.08E−05 | 0.00254245 | 3.25420514 |
| 226389_s_at | RAPGEF1 | 1.09449369 | 10.0900401 | 4.80799583 | 1.09E−05 | 0.00254533 | 3.24920801 |
| 238893_at | LOC338758 | 1.19879171 | 8.45854652 | 4.79847311 | 1.13E−05 | 0.00260733 | 3.21767044 |
| 1559399_s_at | ZCCHC10 | 1.08397924 | 9.324417 | 4.79836233 | 1.13E−05 | 0.00260733 | 3.21730369 |
| 213281_at | JUN | 1.32929059 | 8.21180845 | 4.79539679 | 1.14E−05 | 0.00260733 | 3.20748765 |
| 35150_at | CD40 | 1.0785382 | 10.2548276 | 4.7810706 | 1.20E−05 | 0.00272195 | 3.16010228 |
| 237252_at | THBD | 1.17836626 | 8.93981242 | 4.7780675 | 1.21E−05 | 0.00274002 | 3.15017657 |
| 204908_s_at | BCL3 | 1.15086138 | 7.37503762 | 4.76630513 | 1.26E−05 | 0.00282288 | 3.11132484 |
| 200884_at | CKB | 1.07535296 | 9.92747723 | 4.76403187 | 1.28E−05 | 0.00282288 | 3.10382067 |
| 222996_s_at | CXXC5 | 1.07526087 | 11.470336 | 4.76122287 | 1.29E−05 | 0.0028399 | 3.09455008 |
| 209949_at | NCF2 | 1.0957522 | 12.1194106 | 4.75475484 | 1.32E−05 | 0.00289468 | 3.07321208 |
| 214175_x_at | PDLIM4 | 1.10938746 | 10.0654194 | 4.75115423 | 1.34E−05 | 0.00292045 | 3.06133891 |
| 211924_s_at | PLAUR | 1.11165511 | 9.95057637 | 4.74398245 | 1.37E−05 | 0.00298199 | 3.0377008 |
| 208786_s_at | MAP1LC3B | 1.06211193 | 10.0695592 | 4.71562836 | 1.52E−05 | 0.00321787 | 2.94439234 |
| 211005_at | LAT | 1.08938993 | 7.96306748 | 4.71210161 | 1.54E−05 | 0.00324053 | 2.93280287 |
| 205681_at | BCL2A1 | 1.21178632 | 7.93446379 | 4.70888129 | 1.55E−05 | 0.00326531 | 2.92222355 |
| 239529_at | C5ORF20 | 1.07787194 | 9.92848977 | 4.70651414 | 1.57E−05 | 0.00328034 | 2.91444905 |
| 211564_s_at | PDLIM4 | 1.14599993 | 9.5625589 | 4.69674734 | 1.62E−05 | 0.00338345 | 2.88238905 |
| 216834_at | RGS1 | 1.23977301 | 8.08711615 | 4.69312858 | 1.64E−05 | 0.00341426 | 2.87051745 |
| 205468_s_at | IRF5 | 1.07554402 | 9.22742578 | 4.6798859 | 1.72E−05 | 0.00356532 | 2.82710733 |
| 201465_s_at | JUN | 1.4322588 | 8.69995542 | 4.64844489 | 1.92E−05 | 0.00391212 | 2.72425354 |
| 204059_s_at | ME1 | 1.12987509 | 10.6613264 | 4.64336736 | 1.96E−05 | 0.0039684 | 2.70767146 |
| 219926_at | POPDC3 | 1.14627403 | 7.07974513 | 4.62989868 | 2.06E−05 | 0.00414687 | 2.66372393 |
| 203217_s_at | ST3GAL5 | 1.09010335 | 8.64299026 | 4.61328701 | 2.18E−05 | 0.00431813 | 2.60959806 |
| 203140_at | BCL6 | 1.09370126 | 9.09728992 | 4.60731699 | 2.23E−05 | 0.00435798 | 2.59016685 |
| 223394_at | SERTAD1 | 1.09520636 | 8.8545176 | 4.60661454 | 2.23E−05 | 0.00435798 | 2.58788125 |
| 1555832_s_at | KLF6 | 1.3281372 | 9.3646245 | 4.60196013 | 2.27E−05 | 0.00439877 | 2.57274081 |
| 202181_at | KIAA0247 | 1.06968044 | 10.7870044 | 4.59786925 | 2.30E−05 | 0.00444699 | 2.55943906 |
| 215346_at | CD40 | 1.11448836 | 7.24866952 | 4.58843542 | 2.38E−05 | 0.00458129 | 2.52878443 |
| 1554036_at | ZBTB24 | 1.18706647 | 7.29410595 | 4.57607014 | 2.49E−05 | 0.00473788 | 2.48864674 |
| 227345_at | TNFRSF10D | 1.11676877 | 7.04849796 | 4.57591796 | 2.49E−05 | 0.00473788 | 2.48815304 |
| 238638_at | SLC37A2 | 1.13101655 | 8.61062051 | 4.57336536 | 2.51E−05 | 0.00474087 | 2.47987348 |
| 221841_s_at | KLF4 | 1.10113355 | 10.8767227 | 4.57011234 | 2.54E−05 | 0.00474759 | 2.469325 |
| 219256_s_at | SH3TC1 | 1.11536627 | 7.53451496 | 4.56946119 | 2.54E−05 | 0.00474759 | 2.46721395 |
| 1555950_a_a | CD55 | 1.09152724 | 10.0342225 | 4.56489196 | 2.59E−05 | 0.00479572 | 2.45240406 |
| 204702_s_at | NFE2L3 | 1.12411725 | 7.17327917 | 4.56338707 | 2.60E−05 | 0.00479572 | 2.44752785 |
| 227811_at | FGD3 | 1.06629472 | 10.1971288 | 4.56331445 | 2.60E−05 | 0.00479572 | 2.44729254 |
| 205505_at | GCNT1 | 1.07750113 | 9.23709484 | 4.55594632 | 2.67E−05 | 0.00487889 | 2.42342858 |
| 224397_s_at | TMTC1 /// L [symbol] | 1.0620857 | 9.26567514 | 4.550558 | 2.72E−05 | 0.0049228 | 2.40598784 |
| 201642_at | IFNGR2 | 1.05959086 | 11.6162722 | 4.54471501 | 2.78E−05 | 0.00499333 | 2.38708599 |
| 206508_at | TNFSF7 | 1.05574374 | 11.4883906 | 4.54463083 | 2.78E−05 | 0.00499333 | 2.38681375 |
| 208091_s_at | ECOP | 1.04916375 | 11.1694214 | 4.54122019 | 2.81E−05 | 0.00502048 | 2.37578563 |
| 200797_s_at | MCL1 | 1.07430577 | 12.8237735 | 4.53733476 | 2.85E−05 | 0.00507286 | 2.36322689 |
| 224516_s_at | CXXC5 | 1.06346079 | 12.1814931 | 4.5350953 | 2.87E−05 | 0.0050963 | 2.35599058 |
| 202838_at | FUCA1 | 1.08015905 | 8.59058103 | 4.52575753 | 2.97E−05 | 0.00521504 | 2.32586748 |
| 218902_at | NOTCH1 | 1.07544712 | 10.0281701 | 4.51751651 | 3.05E−05 | 0.00535088 | 2.29924525 |
| 1556314_a_a | — | 1.19283635 | 8.47221892 | 4.51444845 | 3.09E−05 | 0.00539144 | 2.2893517 |
| 212552_at | HPCAL1 | 1.05856719 | 11.9497308 | 4.51088571 | 3.13E−05 | 0.00541251 | 2.27786684 |
| 220307_at | CD244 | 1.06735723 | 10.3372156 | 4.50054668 | 3.24E−05 | 0.00557134 | 2.24456149 |
| 1555827_at | CCNL1 | 1.31890926 | 6.17504609 | 4.49891525 | 3.26E−05 | 0.00558567 | 2.23930934 |
| 205349_at | GNA15 | 1.06857174 | 8.74643664 | 4.49799127 | 3.27E−05 | 0.00558624 | 2.23633511 |
| 210785_s_at | C1ORF38 | 1.10692612 | 9.80722622 | 4.4919076 | 3.34E−05 | 0.00568834 | 2.21675923 |
| 209568_s_at | RGL1 | 1.09184408 | 7.96502675 | 4.49062407 | 3.35E−05 | 0.00568834 | 2.21263071 |
| 212298_at | NRP1 | 1.20491357 | 7.7598722 | 4.49014314 | 3.36E−05 | 0.00568834 | 2.21108391 |
| 223145_s_at | C6ORF166 | 1.05608761 | 11.4884714 | 4.48713202 | 3.40E−05 | 0.00573078 | 2.20140111 |
| 204257_at | FADS3 | 1.09493945 | 8.41951964 | 4.48151372 | 3.46E−05 | 0.0058264 | 2.18334247 |
| 202686_s_at | AXL | 1.09254285 | 8.17667586 | 4.47347755 | 3.56E−05 | 0.0059656 | 2.15753052 |
| 216080_s_at | FADS3 | 1.09835074 | 8.00176994 | 4.46781128 | 3.63E−05 | 0.00601925 | 2.13934359 |
| 223392_s_at | TSHZ3 | 1.06527254 | 7.90879098 | 4.46593878 | 3.66E−05 | 0.00604039 | 2.13333582 |
| 55093_at | CSGLCA-T | 1.06274155 | 9.78511615 | 4.4615716 | 3.71E−05 | 0.00611247 | 2.11964927 |
| 234351_x_at | TRPS1 | 1.08022797 | 8.01237377 | 4.45917263 | 3.74E−05 | 0.00612832 | 2.11163694 |
| 212882_at | KLHL18 | 1.05377934 | 9.17946015 | 4.45890277 | 3.75E−05 | 0.00612832 | 2.11077186 |
| 230698_at | — | 1.13801592 | 5.25817407 | 4.45834849 | 3.75E−05 | 0.00612832 | 2.10899501 |
| 1553142_at | C13ORF31 | 1.22937031 | 5.40269403 | 4.45539446 | 3.79E−05 | 0.0061733 | 2.09952717 |
| 215195_at | PRKCA | 1.07061108 | 8.15580417 | 4.4501435 | 3.86E−05 | 0.0062686 | 2.08270483 |
| 223305_at | MGC13379 | 1.04781874 | 10.291887 | 4.44708307 | 3.91E−05 | 0.00631099 | 2.07290452 |
| 212681_at | EPB41L3 | 1.16483629 | 6.50195379 | 4.44605331 | 3.92E−05 | 0.00631099 | 2.06960767 |
| 212225_at | EIF1 | 1.29978025 | 8.27092573 | 4.42703387 | 4.19E−05 | 0.00665515 | 2.00878036 |
| 235299_at | — | 1.16674528 | 5.45895742 | 4.42210056 | 4.26E−05 | 0.00675057 | 1.993023 |
| 221658_s_at | IL21R | 1.1069911 | 6.7874754 | 4.41756867 | 4.33E−05 | 0.00681813 | 1.97855514 |
| 223276_at | MST150 | 1.04156368 | 11.4183626 | 4.40792637 | 4.47E−05 | 0.00696979 | 1.9477961 |
| 218280_x_at | HIST2H2AA / [symbol] | 1.08743247 | 10.2812164 | 4.40125238 | 4.58E−05 | 0.00711271 | 1.92652478 |
| 218647_s_at | YRDC | 1.06300514 | 9.78941527 | 4.38080776 | 4.92E−05 | 0.00752763 | 1.86146015 |
| 218589_at | P2RY5 | 1.1167465 | 8.63436917 | 4.37437741 | 5.03E−05 | 0.00764929 | 1.84102585 |
| 207535_s_at | NFKB2 | 1.10079929 | 6.90974535 | 4.37295025 | 5.05E−05 | 0.00764929 | 1.8364926 |
| 230925_at | APBB1IP | 1.06629311 | 12.0575087 | 4.35935406 | 5.29E−05 | 0.00795107 | 1.79334155 |
| 48106_at | FLJ20489 | 1.0637033 | 9.15515486 | 4.35698216 | 5.34E−05 | 0.00799448 | 1.78582038 |
| 210872_x_at | GAS7 | 1.08154753 | 7.70819207 | 4.35585799 | 5.36E−05 | 0.00800363 | 1.78225642 |
| 204359_at | FLRT2 | 1.14647669 | 7.31270266 | 4.35162998 | 5.44E−05 | 0.00806509 | 1.76885628 |
| 235457_at | MAML2 | 1.11934624 | 7.88052648 | 4.35157862 | 5.44E−05 | 0.00806509 | 1.76869354 |
| 209239_at | NFKB1 | 1.06667957 | 9.95829149 | 4.34970949 | 5.47E−05 | 0.00808685 | 1.76277161 |
| 57163_at | ELOVL1 | 1.03977111 | 11.2971996 | 4.34572593 | 5.55E−05 | 0.00815484 | 1.75015477 |
| 218145_at | TRIB3 | 1.05938261 | 11.0204954 | 4.33624331 | 5.73E−05 | 0.00835029 | 1.7201439 |
| 211776_s_at | EPB41L3 | 1.13532577 | 6.97220625 | 4.3357415 | 5.74E−05 | 0.00835029 | 1.71855664 |
| 203455_s_at | SAT1 | 1.1106924 | 10.2329144 | 4.32750295 | 5.91E−05 | 0.00843913 | 1.69251066 |
| 207571_x_at | C1ORF38 | 1.09650304 | 9.82311518 | 4.32655202 | 5.93E−05 | 0.00843913 | 1.68950588 |
| 211661_x_at | PTAFR | 1.09097156 | 8.56515505 | 4.32283339 | 6.00E−05 | 0.00852559 | 1.67775875 |
| 201590_x_at | ANXA2 | 1.0821024 | 12.5151345 | 4.29910185 | 6.51E−05 | 0.00910791 | 1.60290884 |
| 202071_at | SDC4 | 1.10346382 | 9.40347252 | 4.29057952 | 6.71E−05 | 0.00933036 | 1.57607916 |
| 208983_s_at | PECAM1 | 1.05734731 | 11.7319933 | 4.28529786 | 6.83E−05 | 0.00945271 | 1.55946497 |
| 205098_at | CCR1 | 1.06303521 | 11.4600607 | 4.28157126 | 6.92E−05 | 0.00952594 | 1.54774856 |
| 200839_s_at | CTSB | 1.0495602 | 12.4472914 | 4.27935833 | 6.97E−05 | 0.00952643 | 1.54079356 |
| 205503_at | PTPN14 | 1.11449101 | 5.88707054 | 4.27156243 | 7.16E−05 | 0.00973541 | 1.51630622 |
| 204998_s_at | ATF5 | 1.09981149 | 9.30480593 | 4.26739042 | 7.26E−05 | 0.00985086 | 1.50321093 |
| 208961_s_at | KLF6 | 1.23937112 | 8.76862028 | 4.26084325 | 7.43E−05 | 0.00997486 | 1.48267336 |
| 211962_s_at | ZFP36L1 | 1.14336043 | 7.45775693 | 4.25626247 | 7.54E−05 | 0.01008195 | 1.46831355 |
| 223502_s_at | TNFSF13B | 1.09556837 | 9.8619397 | 4.25432078 | 7.59E−05 | 0.01008195 | 1.46222911 |
| 212062_at | ATP9A | 1.12049998 | 7.42652014 | 4.24093141 | 7.95E−05 | 0.01044325 | 1.42031066 |
| 222165_x_at | C9ORF16 | 1.06651407 | 9.91796752 | 4.24002979 | 7.97E−05 | 0.01044325 | 1.41749035 |
| 201926_s_at | CD55 | 1.09241207 | 9.80032696 | 4.23706219 | 8.05E−05 | 0.01046218 | 1.40820968 |
| 214446_at | ELL2 | 1.08240974 | 7.5600877 | 4.23640074 | 8.07E−05 | 0.01046218 | 1.40614158 |
| 205718_at | ITGB7 | 1.06295759 | 9.27615896 | 4.23403904 | 8.14E−05 | 0.01049314 | 1.39875869 |
| 216504_s_at | SLC39A8 | 1.10938409 | 7.59501292 | 4.23284649 | 8.17E−05 | 0.01049314 | 1.3950315 |
| 221799_at | CSGLCA-T | 1.07612526 | 9.24334654 | 4.23263401 | 8.18E−05 | 0.01049314 | 1.39436746 |
| 1554406_a_a | CLEC7A | 1.10481365 | 7.94057206 | 4.23125877 | 8.21E−05 | 0.01051774 | 1.39007005 |
| 41644_at | SASH1 | 1.07635742 | 10.9010644 | 4.22769131 | 8.31E−05 | 0.01058985 | 1.3789256 |
| 238669_at | PTGS1 | 1.10119423 | 8.07043819 | 4.21299805 | 8.74E−05 | 0.01101178 | 1.33307571 |
| 211571_s_at | CSPG2 | 1.11087565 | 8.5143978 | 4.21117973 | 8.80E−05 | 0.01105461 | 1.32740739 |
| 203702_s_at | TTLL4 | 1.05712621 | 8.43342998 | 4.2038303 | 9.02E−05 | 0.01130824 | 1.30450953 |
| 209906_at | C3AR1 | 1.12163622 | 7.99304364 | 4.19174628 | 9.40E−05 | 0.01159647 | 1.26690537 |
| 221752_at | SSH1 | 1.06296062 | 8.5565014 | 4.18795524 | 9.52E−05 | 0.011693 | 1.25511957 |
| 204655_at | CCL5 | 1.08067913 | 11.8965338 | 4.18339572 | 9.67E−05 | 0.01179566 | 1.24095201 |
| 1555788_a_a | TRIB3 | 1.07151286 | 9.51438422 | 4.18231045 | 9.70E−05 | 0.01181277 | 1.23758099 |
| 209160_at | AKR1C3 | 1.14905554 | 5.73155063 | 4.17829766 | 9.83E−05 | 0.0118711 | 1.22512056 |
| 224783_at | FAM100B | 1.05774261 | 9.77579353 | 4.17654931 | 9.89E−05 | 0.0118964 | 1.21969356 |
| 203549_s_at | LPL | 1.05114008 | 12.062737 | 4.17631113 | 9.90E−05 | 0.0118964 | 1.21895431 |
| 230343_at | — | 1.0958987 | 7.39528117 | 4.17213744 | 0.00010041 | 0.01203929 | 1.20600404 |
| 201925_s_at | CD55 | 1.12915465 | 9.47556264 | 4.16888144 | 0.00010152 | 0.01212964 | 1.19590588 |
| 214581_x_at | TNFRSF21 | 1.11185517 | 9.56118012 | 4.16612537 | 0.00010247 | 0.01220649 | 1.18736144 |
| 215813_s_at | PTGS1 | 1.06554211 | 10.1951822 | 4.16354389 | 0.00010337 | 0.0122868 | 1.17936091 |
| 39582_at | CYLD | 1.07942531 | 7.63563499 | 4.16212948 | 0.00010387 | 0.01231894 | 1.17497847 |
| 219890_at | CLEC5A | 1.14451027 | 8.86385658 | 4.15970971 | 0.00010472 | 0.01239328 | 1.16748282 |
| 224218_s_at | TRPS1 | 1.10873593 | 7.212934 | 4.15691737 | 0.00010572 | 0.01243643 | 1.15883589 |
| 207275_s_at | ACSL1 | 1.06382835 | 10.2365501 | 4.15687886 | 0.00010573 | 0.01243643 | 1.15871663 |
| 1560485_at | HIVEP1 | 1.09963892 | 6.29901302 | 4.14268989 | 0.00011092 | 0.01282144 | 1.11482542 |
| 224967_at | UGCG | 1.07551513 | 9.81750879 | 4.13803967 | 0.00011267 | 0.01294212 | 1.1004579 |
| 235479_at | CPEB2 | 1.05886438 | 8.76040386 | 4.13334561 | 0.00011447 | 0.01305473 | 1.08596354 |
| 205323_s_at | MTF1 | 1.05603723 | 9.75949747 | 4.13204585 | 0.00011497 | 0.01305473 | 1.08195167 |
| 202643_s_at | TNFAIP3 | 1.09192276 | 8.84841512 | 4.13157653 | 0.00011516 | 0.01305473 | 1.0805032 |
| 202948_at | IL1R1 | 1.11651018 | 6.61849267 | 4.13003859 | 0.00011575 | 0.01305473 | 1.07575731 |
| 205469_s_at | IRF5 | 1.05102073 | 11.1050752 | 4.12871511 | 0.00011627 | 0.01305473 | 1.07167395 |
| 211676_s_at | IFNGR1 | 1.05547086 | 11.597122 | 4.11880246 | 0.00012022 | 0.01335977 | 1.04111224 |
| 226039_at | MGAT4A | 1.06778678 | 10.4027019 | 4.10156128 | 0.0001274 | 0.01404306 | 0.98804884 |
| 213763_at | HIPK2 | 1.10105649 | 7.77301596 | 4.09563418 | 0.00012996 | 0.01425665 | 0.96983433 |
| 222877_at | NRP2 | 1.15705726 | 5.02483818 | 4.09527073 | 0.00013012 | 0.01425665 | 0.96871786 |
| 228186_s_at | RSPO3 | 1.14318575 | 5.7292516 | 4.09151825 | 0.00013176 | 0.01437971 | 0.95719398 |
| 228640_at | — | 1.18540594 | 4.77536947 | 4.08751237 | 0.00013355 | 0.01454523 | 0.94489812 |
| 1558404_at | LOC644242 / [symbol] | 1.23417115 | 5.05041215 | 4.08679741 | 0.00013387 | 0.01455116 | 0.94270427 |
| 1554966_a_a | DOC1 | 1.09595173 | 8.58722147 | 4.0855338 | 0.00013444 | 0.01456531 | 0.93882738 |
| 209803_s_at | PHLDA2 | 1.09168215 | 8.97143045 | 4.08488393 | 0.00013473 | 0.01456531 | 0.93683375 |
| 1555759_a_a | CCL5 | 1.08590053 | 12.3665501 | 4.08473429 | 0.0001348 | 0.01456531 | 0.93637472 |
| 205322_s_at | MTF1 | 1.06116697 | 9.07418068 | 4.07720028 | 0.00013824 | 0.01490832 | 0.91327536 |
| 204197_s_at | RUNX3 | 1.07101524 | 9.2606011 | 4.075793 | 0.0001389 | 0.01494692 | 0.90896318 |
| 224785_at | FAM100B | 1.06028293 | 9.68830224 | 4.07291553 | 0.00014024 | 0.01494692 | 0.90014852 |
| 204011_at | SPRY2 | 1.07921096 | 9.36460861 | 4.06326541 | 0.00014485 | 0.01535236 | 0.87061139 |
| 218673_s_at | ATG7 | 1.0429976 | 10.1275004 | 4.063176 | 0.00014489 | 0.01535236 | 0.87033789 |
| 244434_at | — | 1.20479383 | 5.19908075 | 4.05858303 | 0.00014713 | 0.01555469 | 0.85629319 |
| 203313_s_at | TGIF | 1.07792023 | 9.34545441 | 4.05661779 | 0.0001481 | 0.01555469 | 0.85028636 |
| 214057_at | MCL1 | 1.08299074 | 8.45324848 | 4.05637551 | 0.00014822 | 0.01555469 | 0.84954593 |
| 201473_at | JUNB | 1.18429728 | 8.79340992 | 4.05365798 | 0.00014957 | 0.01564376 | 0.84124255 |
| 203234_at | UPP1 | 1.11194547 | 9.00007417 | 4.05220054 | 0.0001503 | 0.01565768 | 0.83679059 |
| 223126_s_at | C1ORF21 | 1.0962014 | 8.74641324 | 4.05101842 | 0.0001509 | 0.01565768 | 0.83318028 |
| 203604_at | ZNF516 | 1.06761148 | 9.49503774 | 4.05097313 | 0.00015092 | 0.01565768 | 0.83304199 |
| 219398_at | CIDEC | 1.06603998 | 7.6766591 | 4.05011563 | 0.00015135 | 0.01567285 | 0.83042347 |
| 241418_at | — | 1.11426423 | 6.19780118 | 4.04745294 | 0.00015271 | 0.01578293 | 0.82229445 |
| 239845_at | — | 1.14637752 | 5.55035202 | 4.04641564 | 0.00015324 | 0.01580779 | 0.81912842 |
| 214010_s_at | ATP9B | 1.05604217 | 8.49566348 | 4.04162949 | 0.0001557 | 0.01601409 | 0.80452591 |
| 201063_at | RCN1 | 1.05280589 | 9.82235338 | 4.02360178 | 0.00016534 | 0.01680334 | 0.74960812 |
| 226064_s_at | DGAT2 | 1.05258611 | 9.74527668 | 4.00179079 | 0.00017778 | 0.01786795 | 0.68334514 |
| 212419_at | C10ORF56 | 1.07773109 | 8.71242101 | 3.99856112 | 0.0001797 | 0.01799441 | 0.67355005 |
| 201199_s_at | PSMD1 | 1.03812334 | 11.768617 | 3.99425825 | 0.00018228 | 0.01820614 | 0.66050691 |
| 1554691_a_a | PACSIN2 | 1.05020015 | 10.5255625 | 3.98851731 | 0.00018579 | 0.01843526 | 0.64311668 |
| 213503_x_at | ANXA2 | 1.07802407 | 12.4262407 | 3.98512527 | 0.00018789 | 0.01856568 | 0.63284816 |
| 225386_s_at | HNRPLL | 1.04770359 | 11.2270978 | 3.98475235 | 0.00018812 | 0.01856568 | 0.63171954 |
| 219093_at | FLJ20701 | 1.14335282 | 6.49529551 | 3.98380226 | 0.00018871 | 0.01858124 | 0.62884438 |
| 212657_s_at | IL1RN | 1.06064344 | 10.3261013 | 3.97849886 | 0.00019206 | 0.01881842 | 0.61280238 |
| 215411_s_at | TRAF3IP2 | 1.04746131 | 9.60905229 | 3.97606781 | 0.00019361 | 0.01893656 | 0.60545275 |
| 224828_at | CPEB4 | 1.11148606 | 6.94793798 | 3.97135657 | 0.00019665 | 0.01908993 | 0.59121672 |
| 208309_s_at | MALT1 | 1.07563121 | 7.84016779 | 3.95900453 | 0.00020485 | 0.01975315 | 0.55393702 |
| 211219_s_at | LHX2 | 1.07519303 | 8.41763861 | 3.95580414 | 0.00020702 | 0.01992792 | 0.54428847 |
| 208619_at | DDB1 | 1.04094564 | 10.905059 | 3.9493717 | 0.00021147 | 0.02022705 | 0.52490916 |
| 212659_s_at | IL1RN | 1.06314743 | 8.36939546 | 3.94913045 | 0.00021164 | 0.02022705 | 0.52418269 |
| 204401_at | KCNN4 | 1.0819664 | 10.2570594 | 3.94621319 | 0.00021368 | 0.02031839 | 0.51539986 |
| 203922_s_at | CYBB | 1.09189933 | 9.03066782 | 3.94421507 | 0.00021509 | 0.02041722 | 0.50938632 |
| 225955_at | METRNL /// [symbol] | 1.12640184 | 9.30736877 | 3.94080195 | 0.00021753 | 0.02054128 | 0.49911817 |
| 223019_at | C9ORF88 | 1.11519595 | 8.77056793 | 3.93913081 | 0.00021873 | 0.02061912 | 0.49409247 |
| 225372_at | C10ORF54 | 1.11079316 | 7.82555013 | 3.93618776 | 0.00022086 | 0.02074849 | 0.48524461 |
| 225368_at | HIPK2 | 1.10811883 | 10.9436109 | 3.93081197 | 0.00022481 | 0.02104628 | 0.46909267 |
| 227265_at | KIAA1505 | 1.17158338 | 9.78973377 | 3.92552212 | 0.00022875 | 0.02130693 | 0.45321112 |
| 238604_at | ECOP | 1.07570947 | 9.50546921 | 3.92153082 | 0.00023178 | 0.02151518 | 0.44123614 |
| 206171_at | ADORA3 | 1.12468497 | 8.70914475 | 3.92097723 | 0.0002322 | 0.02151785 | 0.43957576 |
| 200748_s_at | FTH1 | 1.09484808 | 13.0211632 | 3.91978982 | 0.00023311 | 0.02156547 | 0.43601482 |
| 204858_s_at | ECGF1 | 1.07328226 | 10.0994132 | 3.91907356 | 0.00023366 | 0.0215798 | 0.43386711 |
| 226534_at | KITLG | 1.07330296 | 9.3627121 | 3.90512624 | 0.00024461 | 0.02229014 | 0.3920904 |
| 225407_at | MBP | 1.05793961 | 11.3278556 | 3.90258448 | 0.00024666 | 0.02243941 | 0.38448614 |
| 216594_x_at | AKR1C1 | 1.08731511 | 8.97660025 | 3.90054274 | 0.00024832 | 0.02255267 | 0.37837983 |
| 242157_at | — | 1.10450816 | 7.85915836 | 3.89900755 | 0.00024957 | 0.02262757 | 0.37378967 |
| 203104_at | CSF1R | 1.066843 | 11.2558738 | 3.89852024 | 0.00024997 | 0.02262757 | 0.37233286 |
| 201471_s_at | SQSTM1 | 1.06817603 | 11.064824 | 3.8911549 | 0.00025608 | 0.02302792 | 0.35032669 |
| 201565_s_at | ID2 | 1.05840108 | 12.351258 | 3.88999177 | 0.00025705 | 0.02306893 | 0.34685365 |
| 201412_at | LRP10 | 1.0522513 | 10.0558306 | 3.8879203 | 0.0002588 | 0.0231589 | 0.34066984 |
| 208982_at | PECAM1 | 1.04940273 | 12.4408294 | 3.88579521 | 0.00026061 | 0.02324451 | 0.33432794 |
| 242302_at | APRIN | 1.09399636 | 6.08366862 | 3.88405566 | 0.0002621 | 0.02333917 | 0.32913805 |
| 201920_at | SLC20A1 | 1.0505388 | 11.1251381 | 3.87844015 | 0.00026696 | 0.02365634 | 0.31239348 |
| 214084_x_at | LOC648998 / [symbol] | 1.07297813 | 9.87462439 | 3.87717059 | 0.00026807 | 0.02367801 | 0.30860978 |
| 202804_at | ABCC1 | 1.05325862 | 10.0332207 | 3.86552597 | 0.00027847 | 0.02439924 | 0.27393816 |
| 228532_at | C1ORF162 | 1.06940145 | 10.9010812 | 3.86494651 | 0.00027899 | 0.02440633 | 0.2722144 |
| 210773_s_at | FPRL1 | 1.096511 | 8.0656375 | 3.86209224 | 0.0002816 | 0.02456545 | 0.26372575 |
| 204620_s_at | CSPG2 | 1.09556927 | 10.356296 | 3.85613369 | 0.00028713 | 0.02491902 | 0.24601655 |
| 227067_x_at | NOTCH2NL | 1.0532533 | 10.0530487 | 3.85320299 | 0.00028989 | 0.02507868 | 0.23731212 |
| 226368_at | CHST11 | 1.07195471 | 9.52648879 | 3.8487656 | 0.00029411 | 0.02532955 | 0.22413996 |
| 213142_x_at | LOC54103 | 1.06311548 | 10.4211815 | 3.84869575 | 0.00029418 | 0.02532955 | 0.22393268 |
| 217992_s_at | EFHD2 | 1.06506856 | 11.6374518 | 3.84358776 | 0.00029911 | 0.02567362 | 0.20878091 |
| 221571_at | TRAF3 | 1.06383739 | 9.65844518 | 3.84232271 | 0.00030035 | 0.02570473 | 0.20503022 |
| 225207_at | PDK4 | 1.13997001 | 6.99602651 | 3.84205117 | 0.00030061 | 0.02570473 | 0.20422522 |
| 201191_at | PITPNA | 1.07930886 | 8.54956136 | 3.83764717 | 0.00030495 | 0.02584057 | 0.19117405 |
| 219412_at | RAB38 | 1.09786171 | 8.08626906 | 3.83442124 | 0.00030817 | 0.02600194 | 0.18161959 |
| 50221_at | TFEB | 1.06697604 | 9.57832583 | 3.83038439 | 0.00031224 | 0.02622405 | 0.16966997 |
| 206140_at | LHX2 | 1.06574569 | 9.81614566 | 3.82420766 | 0.00031857 | 0.02659987 | 0.15140014 |
| 226545_at | CD109 | 1.07280592 | 9.51243783 | 3.82411952 | 0.00031866 | 0.02659987 | 0.15113954 |
| 1552486_s_a | LACTB | 1.08525899 | 8.38629859 | 3.82306652 | 0.00031975 | 0.0266503 | 0.1480267 |
| 228368_at | ARHGAP2O | 1.14164081 | 7.83168691 | 3.81871004 | 0.00032431 | 0.02696861 | 0.13515348 |
| 238727_at | LOC440934 | 1.11497925 | 7.08620645 | 3.8125927 | 0.00033081 | 0.02730892 | 0.11709143 |
| 225283_at | ARRDC4 | 1.04969035 | 9.71669678 | 3.8080796 | 0.00033569 | 0.02759937 | 0.10377692 |
| 201995_at | EXT1 | 1.09076481 | 6.49400167 | 3.80540237 | 0.00033861 | 0.02779804 | 0.09588293 |
| 231496_at | FCAMR | 1.09935645 | 7.04227829 | 3.79899562 | 0.00034571 | 0.02829583 | 0.07700546 |
| 217741_s_at | ZFAND5 | 1.03867699 | 11.247246 | 3.79602954 | 0.00034904 | 0.02848348 | 0.06827223 |
| 226075_at | SPSB1 | 1.09367375 | 6.41796675 | 3.79131798 | 0.0003544 | 0.02879199 | 0.0544079 |
| 1569136_at | MGAT4A | 1.18908532 | 6.30607913 | 3.78721234 | 0.00035914 | 0.02909023 | 0.04233483 |
| AFFX-HUMIS [symbol] | STAT1 | 1.06320305 | 9.76660865 | 3.78614824 | 0.00036038 | 0.02914726 | 0.03920699 |
| 206157_at | PTX3 | 1.13951692 | 9.77811771 | 3.77291403 | 0.00037611 | 0.02998389 | 0.00034936 |
| 204899_s_at | SAP30 | 1.05454489 | 9.10790083 | 3.7723175 | 0.00037683 | 0.02998389 | −0.0014002 |
| 210427_x_at | ANXA2 | 1.07331631 | 12.5352054 | 3.77202707 | 0.00037719 | 0.02398389 | −0.002252 |
| 210357_s_at | SMOX | 1.08311104 | 8.03967009 | 3.7719322 | 0.0003773 | 0.02998389 | −0.0025302 |
| 225530_at | MOBKL2A | 1.04391552 | 10.5141996 | 3.76923042 | 0.0003806 | 0.03009487 | −0.010452 |
| 234976_x_at | SLC4A5 | 1.0654205 | 8.11158064 | 3.76817676 | 0.0003819 | 0.03011937 | −0.0135404 |
| 225115_at | HIPK2 | 1.08936992 | 8.35503513 | 3.76532698 | 0.00038542 | 0.03026716 | −0.0218911 |
| 212561_at | RAB6IP1 | 1.05486737 | 10.6169658 | 3.76463655 | 0.00038628 | 0.03026716 | −0.0239136 |
| 233177_s_at | PNKD | 1.05881097 | 8.90692004 | 3.7603318 | 0.00039167 | 0.03054886 | −0.0365193 |
| 205926_at | IL27RA | 1.08811794 | 8.50082949 | 3.75788045 | 0.00039478 | 0.03074707 | −0.0436938 |
| 203940_s_at | VASH1 | 1.10205097 | 8.93929781 | 3.75557481 | 0.00039772 | 0.03085591 | −0.0504392 |
| 230634_x_at | LOC113179 | 1.06907966 | 7.85469698 | 3.75508795 | 0.00039834 | 0.03085591 | −0.0518633 |
| 219994_at | APBB1IP | 1.07862342 | 8.79352106 | 3.75501728 | 0.00039843 | 0.03085591 | −0.05207 |
| 211139_s_at | NAB1 | 1.07131493 | 8.72031957 | 3.74646937 | 0.00040954 | 0.0314976 | −0.0770544 |
| 201707_at | PEX19 | 1.04709872 | 10.5748471 | 3.74254693 | 0.00041473 | 0.03166964 | −0.0885078 |
| 209933_s_at | CD300A | 1.06497364 | 9.72572801 | 3.74177958 | 0.00041576 | 0.03168935 | −0.0907476 |
| 203233_at | IL4R | 1.07541642 | 8.96600019 | 3.74112983 | 0.00041662 | 0.03168935 | −0.092644 |
| 212724_at | RND3 | 1.10102105 | 7.85276337 | 3.73595709 | 0.0004236 | 0.03207812 | −0.1077338 |
| 218260_at | C19ORF58 | 1.05658126 | 9.52851985 | 3.72698571 | 0.00043597 | 0.03278903 | −0.1338755 |
| 202779_s_at | UBE2S /// L [symbol] | 1.03653716 | 12.7522175 | 3.71585925 | 0.00045179 | 0.03383761 | −0.1662444 |
| 204589_at | NUAK1 | 1.08993672 | 6.70389618 | 3.71372502 | 0.00045488 | 0.0339596 | −0.1724466 |
| 200078_s_at | ATP6V0B | 1.0264003 | 13.5385773 | 3.71309911 | 0.0004558 | 0.0339596 | −0.1742651 |
| 202192_s_at | GAS7 | 1.07358071 | 9.32496946 | 3.71302712 | 0.0004559 | 0.0339596 | −0.1744742 |
| 242705_x_at | — | 1.050541 | 9.41748882 | 3.71038947 | 0.00045976 | 0.03410795 | −0.1821356 |
| 218611_at | IER5 | 1.07040021 | 11.1112341 | 3.70562135 | 0.00046683 | 0.0343816 | −0.1959768 |
| 239598_s_at | AYTL1 | 1.0605954 | 9.78054544 | 3.70535414 | 0.00046723 | 0.0343816 | −0.1967522 |
| 219456_s_at | RIN3 | 1.07330011 | 7.90448799 | 3.70257871 | 0.00047139 | 0.03464126 | −0.2048036 |
| 211572_s_at | SLC23A2 | 1.07618941 | 7.04729666 | 3.70142012 | 0.00047314 | 0.03470886 | −0.2081635 |
| 212502_at | C10ORF22 | 1.04307648 | 9.95312582 | 3.70033342 | 0.00047478 | 0.03475049 | −0.2113144 |
| 201251_at | PKM2 | 1.04329421 | 13.0859336 | 3.69894945 | 0.00047688 | 0.03485776 | −0.2153264 |
| 204135_at | DOC1 | 1.07274599 | 8.83386998 | 3.69578905 | 0.00048172 | 0.03516424 | −0.2244847 |
| 219944_at | RSNL2 | 1.08504226 | 7.05512921 | 3.69481629 | 0.00048322 | 0.03517085 | −0.2273027 |
| 210018_x_at | MALT1 | 1.06841222 | 7.99947523 | 3.69389329 | 0.00048464 | 0.03517085 | −0.229976 |
| 236220_at | — | 1.14920843 | 4.26492324 | 3.69312582 | 0.00048583 | 0.03517085 | −0.2321986 |
| 213935_at | ABHD5 | 1.07822608 | 6.87154542 | 3.69294217 | 0.00048612 | 0.03517085 | −0.2327305 |
| 227420_at | TNFAIP8L1 | 1.06654551 | 8.06189779 | 3.69281506 | 0.00048631 | 0.03517085 | −0.2330985 |
| 219496_at | ANKRD57 | 1.13993106 | 6.53387141 | 3.6910586 | 0.00048905 | 0.03532176 | −0.2381839 |
| 214743_at | CUTL1 | 1.06965625 | 9.94412122 | 3.68791266 | 0.00049398 | 0.0355839 | −0.2472886 |
| 1554544_a_a | MBP | 1.07318708 | 10.1607524 | 3.6870054 | 0.00049541 | 0.03564002 | −0.2499134 |
| 218614_at | C12ORF35 | 1.06691348 | 10.0756991 | 3.68269534 | 0.00050226 | 0.03608547 | −0.2623776 |
| 205147_x_at | NCF4 | 1.03578282 | 11.8404339 | 3.6799098 | 0.00050674 | 0.03617329 | −0.2704283 |
| 223852_s_at | STK40 | 1.07352861 | 8.47342374 | 3.67987641 | 0.00050679 | 0.03617329 | −0.2705248 |
| 204834_at | FGL2 | 1.15453451 | 8.63307396 | 3.67701847 | 0.00051142 | 0.03645638 | −0.2787809 |
| 36994_at | ATP6V0C | 1.03052612 | 13.09078 | 3.6758467 | 0.00051333 | 0.03649744 | −0.2821648 |
| 1555168_a_a | CALN1 | 1.07042924 | 7.3789421 | 3.66717734 | 0.00052769 | 0.03716608 | −0.3071804 |
| 203388_at | ARRB2 | 1.05764941 | 9.38740578 | 3.66559438 | 0.00053035 | 0.03717544 | −0.3117441 |
| 214054_at | DOK2 | 1.07678197 | 11.2259885 | 3.66223375 | 0.00053604 | 0.03752644 | −0.321429 |
| 212096_s_at | MTUS1 | 1.11473269 | 7.49260762 | 3.6528896 | 0.00055218 | 0.03845937 | −0.3483291 |
| 225480_at | C1ORF122 | 1.04930018 | 10.1940995 | 3.65080013 | 0.00055586 | 0.03866588 | −0.3543385 |
| 201700_at | CCND3 | 1.04752925 | 12.8929574 | 3.64778851 | 0.00056119 | 0.03888847 | −0.3629964 |
| 218399_s_at | CDCA4 | 1.03865826 | 10.9812294 | 3.64588211 | 0.00056459 | 0.03907466 | −0.3684747 |
| 212003_at | C1ORF144 | 1.05223728 | 9.96851324 | 3.64185086 | 0.00057185 | 0.03942724 | −0.3800532 |
| 205099_s_at | CCR1 | 1.07264855 | 9.06024264 | 3.63132179 | 0.00059123 | 0.04045726 | −0.4102577 |
| 201422_at | IFI30 | 1.05859805 | 11.1928984 | 3.63012447 | 0.00059347 | 0.04050934 | −0.413689 |
| 227220_at | NFXL1 | 1.06892434 | 8.69171722 | 3.62491347 | 0.00060333 | 0.04097752 | −0.4286146 |
| 213923_at | RAP2B | 1.05181562 | 11.5158708 | 3.62050346 | 0.00061179 | 0.04138559 | −0.4412357 |
| 242538_at | TFDP1 | 1.06598341 | 8.25495776 | 3.61714555 | 0.00061831 | 0.04163319 | −0.4508394 |
| 211855_s_at | SLC25A14 | 1.04783376 | 8.87468082 | 3.61205543 | 0.00062832 | 0.04220328 | −0.4653867 |
| 205462_s_at | HPCAL1 | 1.0537572 | 10.6818738 | 3.60669792 | 0.00063902 | 0.04273699 | −0.4806845 |
| 213649_at | SFRS7 | 1.10439275 | 9.36601754 | 3.60651451 | 0.00063939 | 0.04273699 | −0.4812079 |
| 225701_at | AKNA | 1.052602 | 8.96472073 | 3.60207226 | 0.00064841 | 0.04315666 | −0.4938812 |
| 217691_x_at | SLC16A3 | 1.06195853 | 9.25873814 | 3.60186431 | 0.00064883 | 0.04315666 | −0.4944742 |
| 225375_at | C17ORF32 | 1.04600249 | 10.3975791 | 3.59958371 | 0.00065351 | 0.04336219 | −0.5009765 |
| 206710_s_at | EPB41L3 | 1.10596585 | 7.40730158 | 3.59798029 | 0.00065681 | 0.04347608 | −0.5055465 |
| 204393_s_at | ACPP | 1.05975941 | 8.04387684 | 3.59438387 | 0.00066429 | 0.04381176 | −0.5157924 |
| 244536_at | TP53BP2 | 1.18627513 | 7.50015327 | 3.58918064 | 0.00067525 | 0.04421781 | −0.5306045 |
| 219183_s_at | PSCD4 | 1.04907356 | 10.1644303 | 3.58716888 | 0.00067953 | 0.04438865 | −0.5363279 |
| 226267_at | JDP2 | 1.0662635 | 8.31398002 | 3.5828349 | 0.00068885 | 0.04454185 | −0.548651 |
| 202111_at | SLC4A2 | 1.06505371 | 9.07129828 | 3.58192152 | 0.00069082 | 0.04454185 | −0.5512469 |
| 205128_x_at | PTGS1 | 1.05569773 | 10.4322081 | 3.58162573 | 0.00069147 | 0.04454185 | −0.5520875 |
| 227107_at | — | 1.05069658 | 9.99172727 | 3.57890951 | 0.00069739 | 0.0448057 | −0.5598043 |
| 204489_s_at | CD44 | 1.06303314 | 10.62276 | 3.57845421 | 0.00069838 | 0.04481711 | −0.5610975 |
| 224733_at | CMTM3 | 1.05905507 | 10.6346875 | 3.57586316 | 0.00070409 | 0.04507716 | −0.5684547 |
| 229045_at | SLIC1 | 1.07967634 | 7.89386361 | 3.57233315 | 0.00071192 | 0.04541946 | −0.5784727 |
| 242414_at | QPRT | 1.05189094 | 10.2463166 | 3.56946163 | 0.00071836 | 0.04561726 | −0.5866174 |
| 227948_at | FGD4 | 1.09161868 | 7.2001949 | 3.55996253 | 0.00074006 | 0.04665922 | −0.6135311 |
| 207610_s_at | EMR2 | 1.10912425 | 6.51140397 | 3.55918695 | 0.00074185 | 0.04665922 | −0.6157265 |
| 238389_s_at | — | 1.08307842 | 8.36785016 | 3.55808073 | 0.00074443 | 0.04672969 | −0.6188574 |
| 202295_s_at | CTSH | 1.0545204 | 12.0343467 | 3.55490548 | 0.00075186 | 0.04706777 | −0.6278407 |
| 213134_x_at | BTG3 | 1.04181201 | 11.0553855 | 3.55329658 | 0.00075565 | 0.04706777 | −0.6323906 |
| 204495_s_at | C15ORF39 | 1.06073223 | 9.19429662 | 3.55321693 | 0.00075584 | 0.04706777 | −0.6326159 |
| 225631_at | KIAA1706 | 1.09137745 | 7.01050123 | 3.55021925 | 0.00076296 | 0.04734911 | −0.6410896 |
| 217168_s_at | HERPUD1 | 1.03983791 | 11.6835542 | 3.54664359 | 0.00077153 | 0.04764859 | −0.6511914 |
| 203658_at | SLC25A20 | 1.04561479 | 9.46245075 | 3.54654461 | 0.00077177 | 0.04764859 | −0.6514709 |
| 226735_at | FLJ90013 | 1.05055665 | 9.2921795 | 3.54448273 | 0.00077675 | 0.04781967 | −0.657293 |
| 212372_at | MYH10 | 1.06171907 | 9.0082261 | 3.53720879 | 0.00079459 | 0.04864948 | −0.6778151 |
| 214769_at | — | 1.06476746 | 8.52685941 | 3.53521138 | 0.00079955 | 0.04873518 | −0.6834457 |
| 225408_at | MBP | 1.0628553 | 8.95772417 | 3.53415379 | 0.00080219 | 0.04881373 | −0.6864262 |
| 222900_at | — | 1.05686661 | 9.96719959 | 3.53260926 | 0.00080606 | 0.04891403 | −0.690778 |
| 1562475_at | DKFZP686O1 | 1.08348486 | 8.1338617 | 3.53084868 | 0.0008105 | 0.04912864 | −0.6957371 |
| 226629_at | SLC43A2 | 1.09818747 | 7.92209671 | 3.5269652 | 0.00082036 | 0.04956322 | −0.7066702 |
| 204174_at | ALOX5AP | 1.04445917 | 12.5979593 | 3.52653085 | 0.00082147 | 0.04956322 | −0.7078925 |
| 211067_s_at | GAS7 | 1.06066251 | 9.58135451 | 3.52629694 | 0.00082207 | 0.04956322 | −0.7085508 |
| 204157_s_at | KIAA0999 | 1.06020492 | 8.89537432 | 3.52624693 | 0.0008222 | 0.04956322 | −0.7086915 |
| 211026_s_at | MGLL | 1.05773402 | 7.85103089 | 3.52322146 | 0.00082998 | 0.04986733 | −0.7172027 |
| 204614_at | SERPINB2 | −1.1647768 | 12.4744252 | −7.4528925 | 4.60E−10 | 9.68E−07 | 12.5679641 |
| 211421_s_at | RET | −1.1345252 | 9.80328848 | −7.1113218 | 1.75E−09 | 3.41E−06 | 11.3371247 |
| 207725_at | POU4F2 | −1.1644052 | 9.97790101 | −6.9339707 | 3.49E−09 | 6.15E−06 | 10.6980464 |
| 208206_s_at | RASGRP2 | −1.1124445 | 10.5120774 | −6.549181 | 1.56E−08 | 1.82E−05 | 9.31420762 |
| 218971_s_at | HSPC049 | −1.1502366 | 9.26509596 | −6.353404 | 3.32E−08 | 3.04E−05 | 8.6128924 |
| 222799_at | HSPC049 | −1.1678019 | 8.76549992 | −6.2405739 | 5.13E−08 | 4.25E−05 | 8.20995923 |
| 219463_at | C20ORF103 | −1.1066514 | 11.1578088 | −6.1792066 | 6.49E−08 | 5.22E−05 | 7.99126082 |
| 218858_at | DEPDC6 | −1.1739709 | 8.39313128 | −6.1048654 | 8.64E−08 | 6.72E−05 | 7.72680015 |
| 214539_at | SERPINB10 | −1.1393961 | 9.05047781 | −6.0524221 | 1.06E−07 | 7.91E−05 | 7.54057412 |
| 225510_at | OAF | −1.0795282 | 10.1352866 | −5.9010298 | 1.88E−07 | 0.00012109 | 7.0047035 |
| 228855_at | LOC645919 | −1.1276666 | 10.7515984 | −5.8846827 | 2.00E−07 | 0.0001259 | 6.94700615 |
| 200799_at | HSPA1A | −1.0946162 | 10.8594511 | −5.7859004 | 2.92E−07 | 0.00016854 | 6.59909626 |
| 206643_at | HAL | −1.1136035 | 9.45516733 | −5.7636924 | 3.17E−07 | 0.000177 | 6.52106329 |
| 44790_s_at | C13ORF18 | −1.1118604 | 11.1923769 | −5.7586514 | 3.23E−07 | 0.00017859 | 6.50336066 |
| 202441_at | SPFH1 | −1.0851805 | 11.4291926 | −5.6315658 | 5.23E−07 | 0.00025521 | 6.05830953 |
| 206034_at | SERPINB8 | −1.080288 | 10.6134257 | −5.5518212 | 7.06E−07 | 0.00032156 | 5.78034465 |
| 203685_at | BCL2 | −1.1572394 | 9.68186938 | −5.5344339 | 7.53E−07 | 0.00034041 | 5.71987917 |
| 216733_s_at | GATM | −1.0919977 | 10.6962606 | −5.5025531 | 8.49E−07 | 0.00037436 | 5.60914785 |
| 224428_s_at | CDCA7 | −1.0723261 | 11.8097904 | −5.4896239 | 8.91E−07 | 0.0003867 | 5.56429181 |
| 201310_s_at | C5ORF13 | −1.1126729 | 11.785614 | −5.4263444 | 1.13E−06 | 0.00045596 | 5.34518848 |
| 213361_at | TDRD7 | −1.1610606 | 10.9747672 | −5.391919 | 1.28E−06 | 0.00050496 | 5.22630402 |
| 219714_s_at | CACNA2D3 | −1.0949691 | 10.9314712 | −5.3685146 | 1.40E−06 | 0.00054258 | 5.14560908 |
| 35974_at | LRMP | −1.1227255 | 8.47924836 | −5.3181301 | 1.69E−06 | 0.00062826 | 4.97225553 |
| 201309_x_at | C5ORF13 | −1.1203829 | 9.89153948 | −5.2776918 | 1.96E−06 | 0.00070587 | 4.83349392 |
| 243824_at | — | −1.1826717 | 8.98902905 | −5.2561294 | 2.13E−06 | 0.00073085 | 4.75964206 |
| 226117_at | TIFA | −1.0706362 | 10.6431699 | −5.2203927 | 2.43E−06 | 0.0008154 | 4.63745968 |
| 210986_s_at | TPM1 | −1.0953979 | 11.1982 | −5.1779859 | 2.84E−06 | 0.00090145 | 4.49283173 |
| 235275_at | OXCT2 | −1.167218 | 12.4747234 | −5.1258467 | 3.43E−06 | 0.0010376 | 4.31556088 |
| 203178_at | GATM | −1.1772979 | 9.84150408 | −5.0826986 | 4.02E−06 | 0.00116392 | 4.16933171 |
| 223062_s_at | PSAT1 | −1.0966737 | 10.8142971 | −5.0731123 | 4.17E−06 | 0.00119913 | 4.13690304 |
| 227921_at | — | −1.0939212 | 9.03183919 | −5.0296725 | 4.88E−06 | 0.00137633 | 3.99023064 |
| 225673_at | MYADM | −1.0710199 | 12.4026311 | −5.0110829 | 5.23E−06 | 0.00143582 | 3.92760413 |
| 210987_x_at | TPM1 | −1.0747681 | 11.0724469 | −5.008451 | 5.28E−06 | 0.0014424 | 3.91874425 |
| 220252_x_at | CXORF21 | −1.1107929 | 11.2367599 | −4.990621 | 5.63E−06 | 0.00153146 | 3.85876875 |
| 212175_s_at | AK2 | −1.0635824 | 12.0939996 | −4.9479609 | 6.57E−06 | 0.00172797 | 3.71559637 |
| 213931_at | ID2 /// ID2B | −1.5575806 | 8.63155658 | −4.8451866 | 9.53E−06 | 0.00231585 | 3.37261839 |
| 206116_s_at | TPM1 | −1.0886322 | 10.541027 | −4.8397732 | 9.72E−06 | 0.00235097 | 3.35463151 |
| 225421_at | ACY1L2 | −1.1026709 | 10.2401371 | −4.8246433 | 1.03E−05 | 0.00244373 | 3.30440211 |
| 228188_at | — | −1.0980344 | 9.30046163 | −4.8130876 | 1.07E−05 | 0.00252076 | 3.26608136 |
| 1553436_at | MUC19 | −1.1317348 | 7.84569387 | −4.796243 | 1.14E−05 | 0.00260733 | 3.21028822 |
| 223165_s_at | IHPK2 | −1.0812006 | 8.49238402 | −4.7744158 | 1.23E−05 | 0.00276466 | 3.13811043 |
| 217521_at | — | −1.0843722 | 8.45544985 | −4.7662509 | 1.27E−05 | 0.00282288 | 3.11114575 |
| 214787_at | DENND4A | −1.1130314 | 8.79367689 | −4.743088 | 1.37E−05 | 0.00298199 | 3.03475381 |
| 212765_at | CAMSAP1L1 | −1.0863982 | 9.42505575 | −4.7345547 | 1.42E−05 | 0.0030621 | 3.00664977 |
| 210254_at | MS4A3 | −1.0500477 | 12.8157054 | −4.7261168 | 1.46E−05 | 0.00314327 | 2.9788805 |
| 233176_at | — | −1.1278256 | 8.19196857 | −4.7198778 | 1.49E−05 | 0.00318888 | 2.95836149 |
| 223344_s_at | MS4A7 | −1.0542376 | 11.6140787 | −4.6615341 | 1.84E−05 | 0.00376871 | 2.7670363 |
| 219951_s_at | C20ORF12 | −1.0986972 | 9.36939485 | −4.6610806 | 1.84E−05 | 0.00376871 | 2.76555328 |
| 224358_s_at | MS4A7 | −1.0737805 | 10.2934393 | −4.6288589 | 2.06E−05 | 0.00414687 | 2.66033365 |
| 209822_s_at | VLDLR | −1.0809314 | 10.4822858 | −4.6256973 | 2.09E−05 | 0.00417816 | 2.65002641 |
| 1554892_a_a | MS4A3 | −1.0607494 | 11.9760309 | −4.6180329 | 2.14E−05 | 0.00427729 | 2.62505295 |
| 224916_at | LOC340061 | −1.0659077 | 11.5293213 | −4.6084907 | 2.22E−05 | 0.00435798 | 2.59398619 |
| 219607_s_at | MS4A4A | −1.0618962 | 10.8982797 | −4.6084529 | 2.22E−05 | 0.00435798 | 2.59386325 |
| 220112_at | ANKRD55 | −1.0892108 | 8.61196167 | −4.6022914 | 2.27E−05 | 0.00439877 | 2.57381817 |
| 202158_s_at | CUGBP2 | −1.071327 | 10.2596168 | −4.5821696 | 2.43E−05 | 0.00466718 | 2.50843969 |
| 229638_at | IRX3 | −1.0842058 | 12.4826855 | −4.5731157 | 2.51E−05 | 0.00474087 | 2.47906391 |
| 1557359_at | LOC285758 | −1.0922204 | 9.92033734 | −4.5727867 | 2.51E−05 | 0.00474087 | 2.47799688 |
| 1555037_a_a | IDH1 | −1.0626668 | 11.297782 | −4.562743 | 2.61E−05 | 0.00479572 | 2.4454411 |
| 235936_at | LOC254559 | −1.0942156 | 7.69992263 | −4.5533045 | 2.69E−05 | 0.0048917 | 2.41487632 |
| 217196_s_at | CAMSAP1L1 | −1.0905855 | 8.32441354 | −4.5428721 | 2.79E−05 | 0.0050078 | 2.38112639 |
| 225786_at | LOC284702 | −1.1841367 | 9.55400971 | −4.510983 | 3.12E−05 | 0.00541251 | 2.27818048 |
| 203221_at | TLE1 | −1.0725652 | 10.2194004 | −4.504306 | 3.20E−05 | 0.00551587 | 2.25606737 |
| 218507_at | HIG2 | −1.0603259 | 10.2529741 | −4.4729944 | 3.57E−05 | 0.0059656 | 2.1559794 |
| 223952_x_at | DHRS9 | −1.0842606 | 12.9366028 | −4.4456627 | 3.92E−05 | 0.00631099 | 2.0683571 |
| 224929_at | LOC340061 | −1.0551654 | 11.6690807 | −4.4386464 | 4.02E−05 | 0.00644802 | 2.04590464 |
| 209485_s_at | OSBPL1A | −1.0832594 | 8.89666819 | −4.4376043 | 4.04E−05 | 0.00645252 | 2.04257108 |
| 222369_at | NAT11 | −1.0773853 | 9.63846481 | −4.4294999 | 4.15E−05 | 0.00661759 | 2.01666018 |
| 210279_at | GPR18 | −1.1061684 | 9.71898614 | −4.4206221 | 4.28E−05 | 0.00676572 | 1.98830241 |
| 206542_s_at | SMARCA2 | −1.0665392 | 10.093152 | −4.4164022 | 4.34E−05 | 0.00682612 | 1.97483232 |
| 228055_at | NAPSB | −1.1406853 | 10.0636876 | −4.411431 | 4.42E−05 | 0.00690524 | 1.95897232 |
| 206088_at | LOC474170 | −1.1642536 | 5.60881351 | −4.3987993 | 4.62E−05 | 0.00715313 | 1.91871021 |
| 1555728_a_a | MS4A4A | −1.0619928 | 10.814275 | −4.3884831 | 4.79E−05 | 0.00739242 | 1.8858698 |
| 229159_at | KIAA0960 | −1.1638873 | 7.81943811 | −4.378427 | 4.96E−05 | 0.00756868 | 1.85389295 |
| 212071_s_at | SPTBN1 | −1.0802737 | 10.3524809 | −4.3710543 | 5.08E−05 | 0.00767831 | 1.83047131 |
| 224009_x_at | DHRS9 | −1.0883843 | 12.9995564 | −4.3451551 | 5.56E−05 | 0.00815484 | 1.74834735 |
| 219471_at | C13ORF18 | −1.082994 | 11.244718 | −4.3449394 | 5.56E−05 | 0.00815484 | 1.74766447 |
| 204674_at | LRMP | −1.0754387 | 9.48836985 | −4.3365053 | 5.73E−05 | 0.00835029 | 1.72097259 |
| 225594_at | ZF | −1.0660997 | 8.44053993 | −4.3348185 | 5.76E−05 | 0.00835466 | 1.71563729 |
| 64900_at | CHST5 /// A [symbol] | −1.0783605 | 7.45420385 | −4.3330734 | 5.80E−05 | 0.00836918 | 1.71011893 |
| 218326_s_at | LGR4 | −1.1055698 | 7.35402657 | −4.3327777 | 5.80E−05 | 0.00836918 | 1.70918383 |
| 204639_at | ADA | −1.0663173 | 8.71406105 | −4.3311807 | 5.83E−05 | 0.00839318 | 1.70413466 |
| 220416_at | ATP8B4 | −1.0560097 | 10.8651277 | −4.3288715 | 5.88E−05 | 0.00843796 | 1.69683558 |
| 226255_at | ZBTB33 | −1.056353 | 11.34447 | −4.3186727 | 6.09E−05 | 0.00862606 | 1.66462107 |
| 204548_at | STAR | −1.0828983 | 8.71225533 | −4.3131689 | 6.21E−05 | 0.00876809 | 1.64725213 |
| 207865_s_at | BMP8B | −1.1309201 | 9.46800068 | −4.3117307 | 6.24E−05 | 0.00878882 | 1.64271524 |
| 244447_at | KLF10 | −1.3134824 | 7.8071422 | −4.2828862 | 6.89E−05 | 0.00950707 | 1.55188213 |
| 224837_at | FOXP1 | −1.0552487 | 11.2859527 | −4.279807 | 6.96E−05 | 0.00952643 | 1.54220361 |
| 201689_s_at | TPD52 | −1.0726713 | 9.3965084 | −4.2740096 | 7.10E−05 | 0.00967829 | 1.52399041 |
| 212097_at | CAV1 | −1.0973744 | 8.37508938 | −4.2610503 | 7.42E−05 | 0.00997486 | 1.48332253 |
| 1557570_a_a | LOC285084 | −1.0977131 | 8.27009497 | −4.2541445 | 7.60E−05 | 0.01008195 | 1.46167694 |
| 201690_s_at | TPD52 | −1.073115 | 10.5093023 | −4.2504384 | 7.69E−05 | 0.01016104 | 1.45006755 |
| 214920_at | KIAA0960 | −1.1291766 | 9.28547078 | −4.2405995 | 7.96E−05 | 0.01044325 | 1.41927233 |
| 226431_at | ALS2CR13 | −1.0804181 | 8.88019612 | −4.2395927 | 7.98E−05 | 0.01044325 | 1.4161231 |
| 227629_at | PRLR | −1.1385104 | 7.04094281 | −4.2375147 | 8.04E−05 | 0.01046218 | 1.40962463 |
| 57540_at | RBKS | −1.0889358 | 8.11543924 | −4.2362686 | 8.08E−05 | 0.01046218 | 1.40572847 |
| 228455_at | SLC16A4 | −1.2438773 | 7.61141506 | −4.2334573 | 8.15E−05 | 0.01049314 | 1.39694035 |
| 242511_at | ST3GAL3 | −1.0989987 | 7.9960706 | −4.2291028 | 8.27E−05 | 0.01057053 | 1.38333428 |
| 225619_at | SLAIN1 | −1.1432234 | 8.6802401 | −4.227198 | 8.33E−05 | 0.01058985 | 1.37738486 |
| 213737_x_at | GOLGA8G // [symbol] | −1.0557244 | 13.4113502 | −4.2214214 | 8.49E−05 | 0.01077515 | 1.35935067 |
| 224486_s_at | C15ORF41 | −1.0809669 | 9.42072522 | −4.2192321 | 8.56E−05 | 0.01083061 | 1.35251877 |
| 230480_at | PIWIL4 | −1.0659842 | 10.6705451 | −4.2178649 | 8.60E−05 | 0.01085599 | 1.34825366 |
| 225224_at | C20ORF112 | −1.0767769 | 9.19255558 | −4.1971114 | 9.23E−05 | 0.01151643 | 1.28359413 |
| 219799_s_at | DHRS9 | −1.0873546 | 12.5296658 | −4.1953591 | 9.28E−05 | 0.01155875 | 1.27814225 |
| 202979_s_at | ZF | −1.0920673 | 9.89384482 | −4.1942804 | 9.31E−05 | 0.01157478 | 1.27478664 |
| 206461_x_at | MT1H | −1.0392307 | 11.9250301 | −4.1935006 | 9.34E−05 | 0.01157914 | 1.27236118 |
| 201675_at | AKAP1 | −1.0615779 | 10.8983992 | −4.1859946 | 9.58E−05 | 0.01174461 | 1.24902648 |
| 227949_at | PHACTR3 | −1.0599385 | 10.7921228 | −4.1808322 | 9.75E−05 | 0.01184572 | 1.2329899 |
| 237265_at | C16ORF73 | −1.0739194 | 9.90657205 | −4.1786481 | 9.82E−05 | 0.0118711 | 1.22620861 |
| 235758_at | PNMA6A | −1.0679142 | 8.36962585 | −4.1782399 | 9.84E−05 | 0.0118711 | 1.22494122 |
| 1554298_a_a | WDR49 | −1.168549 | 7.28424515 | −4.1686361 | 0.00010161 | 0.01212964 | 1.19514511 |
| 230008_at | KIAA0960 | −1.1023476 | 9.40901508 | −4.156767 | 0.00010577 | 0.01243643 | 1.15837023 |
| 215771_x_at | RET | −1.0639479 | 8.73407172 | −4.1514318 | 0.00010769 | 0.01258151 | 1.14185786 |
| 204301_at | KBTBD11 | −1.0665649 | 10.3597689 | −4.1465423 | 0.00010949 | 0.01271022 | 1.12673448 |
| 201036_s_at | HADH | −1.0465759 | 10.8776177 | −4.1465262 | 0.00010949 | 0.01271022 | 1.12668456 |
| 223044_at | SLC40A1 | −1.0799265 | 8.72509702 | −4.1435626 | 0.00011059 | 0.01281082 | 1.11752261 |
| 205996_s_at | AK2 | −1.0533596 | 11.6545132 | −4.1314862 | 0.00011519 | 0.01305473 | 1.08022438 |
| 213894_at | KIAA0960 | −1.0975098 | 9.74897241 | −4.1307312 | 0.00011549 | 0.01305473 | 1.07789445 |
| 235823_at | LOC197322 | −1.0867032 | 7.00344771 | −4.1303782 | 0.00011562 | 0.01305473 | 1.07680534 |
| 212593_s_at | PDCD4 | −1.0784075 | 9.6202895 | −4.1277406 | 0.00011665 | 0.01305473 | 1.06866759 |
| 227254_at | — | −1.0878832 | 8.8387562 | −4.1270419 | 0.00011693 | 0.01305473 | 1.06651244 |
| 218082_s_at | UBP1 | −1.061315 | 11.2875622 | −4.1236056 | 0.00011829 | 0.01317223 | 1.05591605 |
| 221648_s_at | C1ORF121 | −1.0625223 | 9.30594001 | −4.1122294 | 0.00012291 | 0.01363084 | 1.02086816 |
| 225883_at | ATG16L2 | −1.0866743 | 8.95908959 | −4.1072117 | 0.000125 | 0.01380676 | 1.0054261 |
| 222388_s_at | VPS35 | −1.0544103 | 10.9995649 | −4.096001 | 0.0001298 | 0.01425665 | 0.9709611 |
| 208636_at | ACTN1 | −1.0393569 | 12.61059 | −4.0933116 | 0.00013097 | 0.01432201 | 0.96270059 |
| 204165_at | WASF1 | −1.0621996 | 10.2583762 | −4.0742957 | 0.0001396 | 0.01494692 | 0.9043759 |
| 239963_at | — | −1.2400451 | 9.0375203 | −4.0742228 | 0.00013963 | 0.01494692 | 0.9041527 |
| 227055_at | METTL7B | −1.0860464 | 10.9882916 | −4.0733051 | 0.00014006 | 0.01494692 | 0.90134178 |
| 229700_at | LOC148203 | −1.0769984 | 9.16397658 | −4.0732431 | 0.00014009 | 0.01494692 | 0.90115181 |
| 225720_at | SYNPO2 | −1.093531 | 8.22812673 | −4.0689427 | 0.00014212 | 0.01511757 | 0.88798401 |
| 233782_at | WDR68 | −1.0892946 | 7.81601785 | −4.0571162 | 0.00014785 | 0.01555469 | 0.85180964 |
| 226438_at | MTBP | −1.0674478 | 9.28796451 | −4.0535202 | 0.00014964 | 0.01564376 | 0.84082167 |
| 200672_x_at | SPTBN1 | −1.0717572 | 7.97282842 | −4.0518568 | 0.00015048 | 0.01565768 | 0.83574084 |
| 228625_at | CITED4 | −1.0665343 | 12.4781742 | −4.0414018 | 0.00015582 | 0.01601409 | 0.80383153 |
| 223704_s_at | DMRT2 | −1.0966738 | 7.49852282 | −4.038741 | 0.00015721 | 0.01612655 | 0.79571762 |
| 223136_at | AIG1 | −1.0602864 | 11.4123181 | −4.0340714 | 0.00015968 | 0.01634893 | 0.78148555 |
| 218168_s_at | CABC1 | −1.05636 | 10.9814373 | −4.0298906 | 0.00016192 | 0.01654732 | 0.76875062 |
| 219056_at | RNASEH2B | −1.0587835 | 9.59474393 | −4.0293131 | 0.00016223 | 0.01654826 | 0.76699214 |
| 1553955_at | LOC129285 | −1.0626958 | 9.65678818 | −4.0249191 | 0.00016462 | 0.01676095 | 0.75361657 |
| 216336_x_at | MT1M | −1.0417674 | 11.497183 | −4.0083934 | 0.00017392 | 0.01757723 | 0.70338342 |
| 209007_s_at | C1ORF63 | −1.0860359 | 10.6932184 | −4.0033033 | 0.00017689 | 0.01782313 | 0.68793375 |
| 204959_at | MNDA | −1.0681514 | 11.9635641 | −4.003101 | 0.00017701 | 0.01782313 | 0.68732013 |
| 232722_at | RNASET2 | −1.121936 | 6.4751986 | −3.9998645 | 0.00017892 | 0.0179496 | 0.67750249 |
| 201924_at | AFF1 | −1.0636204 | 10.9900787 | −3.9938919 | 0.0001825 | 0.01820614 | 0.65939689 |
| 205268_s_at | ADD2 | −1.0749965 | 8.29295849 | −3.9894046 | 0.00018524 | 0.01841452 | 0.64580364 |
| 217892_s_at | LIMA1 | −1.0674835 | 9.77391533 | −3.9742552 | 0.00019477 | 0.0190165 | 0.59997453 |
| 225610_at | UHRF2 | −1.0478186 | 11.8006338 | −3.9712363 | 0.00019673 | 0.01908993 | 0.5908533 |
| 213888_s_at | TRAF3IP3 | −1.0732522 | 10.3053256 | −3.9709396 | 0.00019692 | 0.01908993 | 0.58995707 |
| 209879_at | SELPLG | −1.0891682 | 9.91966583 | −3.9695775 | 0.00019781 | 0.0191422 | 0.58584343 |
| 226630_at | C14ORF106 | −1.0672553 | 9.79100795 | −3.961139 | 0.00020341 | 0.01964899 | 0.56037434 |
| 225455_at | TADA1L | −1.0580187 | 10.2337996 | −3.9517012 | 0.00020985 | 0.02016419 | 0.5319252 |
| 1554352_s_a | DENND4A | −1.0590459 | 9.89217484 | −3.9498502 | 0.00021113 | 0.02022705 | 0.52635021 |
| 227805_at | — | −1.0725811 | 7.97633184 | −3.9486352 | 0.00021198 | 0.02022705 | 0.52269151 |
| 204849_at | TCFL5 | −1.0508793 | 10.0777273 | −3.9473794 | 0.00021286 | 0.02027564 | 0.51891058 |
| 212174_at | AK2 | −1.0435458 | 12.5602536 | −3.9427886 | 0.00021611 | 0.02047123 | 0.50509441 |
| 243431_at | BTBD14A | −1.1988342 | 7.54359435 | −3.9340968 | 0.00022239 | 0.02085605 | 0.4789607 |
| 1558027_s_a | PRKAB2 | −1.0672869 | 8.44162128 | −3.9261915 | 0.00022825 | 0.02129633 | 0.45522005 |
| 220615_s_at | MLSTD1 | −1.0704751 | 11.0839465 | −3.9246829 | 0.00022939 | 0.0213295 | 0.45069271 |
| 220116_at | KCNN2 | −1.0475679 | 10.7237831 | −3.9161743 | 0.00023589 | 0.02174968 | 0.42517604 |
| 201193_at | IDH1 | −1.0387181 | 11.9734806 | −3.9122575 | 0.00023895 | 0.02199427 | 0.41344031 |
| 225782_at | MSRB3 | −1.0704 | 8.85357731 | −3.9114057 | 0.00023962 | 0.02201882 | 0.41088889 |
| 218270_at | MRPL24 | −1.041775 | 10.7168033 | −3.9080124 | 0.0002423 | 0.02219357 | 0.40072844 |
| 1559139_at | NOC2L | −1.1614047 | 7.69426499 | −3.9079762 | 0.00024233 | 0.02219357 | 0.4006202 |
| 201841_s_at | HSPB1 | −1.0441743 | 13.4381243 | −3.9073471 | 0.00024283 | 0.02220226 | 0.39873692 |
| 1569189_at | TTC9C | −1.1233947 | 6.22264013 | −3.9060463 | 0.00024387 | 0.02226003 | 0.39484371 |
| 205944_s_at | CLTCL1 | −1.0767087 | 9.59502778 | −3.8960537 | 0.000252 | 0.0227332 | 0.36496078 |
| 218532_s_at | FLJ20152 | −1.0975951 | 7.14515674 | −3.8958965 | 0.00025213 | 0.0227332 | 0.36449103 |
| 205659_at | HDAC9 | −1.0805755 | 9.52806368 | −3.8955883 | 0.00025238 | 0.0227332 | 0.36356988 |
| 228056_s_at | NAPSB | −1.133171 | 8.05318337 | −3.8864469 | 0.00025006 | 0.02323287 | 0.33627243 |
| 208158_s_at | OSBPL1A | −1.0689057 | 8.96913414 | −3.8834664 | 0.0002626 | 0.0233462 | 0.32738037 |
| 241024_at | C6ORF147 | −1.130088 | 5.62544192 | −3.8775071 | 0.00026777 | 0.02367801 | 0.30961257 |
| 212859_x_at | MT1E | −1.0360762 | 12.3752245 | −3.8735521 | 0.00027126 | 0.02392114 | 0.29782945 |
| 219498_s_at | BCL11A | −1.0732492 | 8.13793041 | −3.8714456 | 0.00027313 | 0.0240476 | 0.29155627 |
| 215095_at | ESD | −1.1487406 | 5.82227073 | −3.8696858 | 0.00027471 | 0.02414738 | 0.28631705 |
| 203253_s_at | HISPPD1 | −1.0681144 | 10.4329202 | −3.8669778 | 0.00027715 | 0.02432282 | 0.2782577 |
| 213416_at | ITGA4 | −1.0669742 | 10.3810332 | −3.8619771 | 0.00028171 | 0.02456545 | 0.26338338 |
| 1557283_a_a | ZNF519 | −1.1068414 | 5.33423642 | −3.8586267 | 0.00028481 | 0.02479593 | 0.25342411 |
| 230424_at | C5ORF13 | −1.129586 | 6.85369311 | −3.8555889 | 0.00028764 | 0.02492377 | 0.24439825 |
| 230550_at | MS4A6A | −1.0527231 | 11.5245536 | −3.8469498 | 0.00029586 | 0.02543398 | 0.21875242 |
| 1555963_x_a | B3GNT7 | −1.0971417 | 7.71455009 | −3.8417726 | 0.00030089 | 0.02570473 | 0.20339936 |
| 203518_at | LYST | −1.0508267 | 12.5285349 | −3.8410237 | 0.00030162 | 0.02572727 | 0.20117947 |
| 229084_at | CNTN4 | −1.1046924 | 8.60627464 | −3.8396872 | 0.00030294 | 0.02579917 | 0.19721855 |
| 200912_s_at | EIF4A2 | −1.0342758 | 13.3848401 | −3.8372855 | 0.00030531 | 0.02584057 | 0.19010257 |
| 243514_at | — | −1.1677325 | 8.64926127 | −3.834963 | 0.00030763 | 0.02599628 | 0.18322394 |
| 1560495_at | — | −1.0868663 | 7.91686834 | −3.8289949 | 0.00031366 | 0.02626207 | 0.16555845 |
| 209539_at | ARHGEF6 | −1.0428012 | 11.4186716 | −3.8184713 | 0.00032456 | 0.02696861 | 0.13444828 |
| 211980_at | COL4A1 | −1.0583579 | 8.52391992 | −3.8156429 | 0.00032755 | 0.02717595 | 0.1260953 |
| 222538_s_at | APPL | −1.0538895 | 9.69059359 | −3.8135376 | 0.0003298 | 0.02730892 | 0.11988026 |
| 217540_at | — | −1.0829555 | 9.09428634 | −3.8128923 | 0.00033049 | 0.02730892 | 0.11797567 |
| 231950_at | ZNF658 | −1.0895471 | 8.18062639 | −3.8089596 | 0.00033473 | 0.02756221 | 0.10637231 |
| 221900_at | COL8A2 | −1.0731684 | 10.4949814 | −3.8010969 | 0.00034336 | 0.02814612 | 0.0831949 |
| 229033_s_at | MUM1 | −1.0955974 | 7.47621492 | −3.798449 | 0.00034632 | 0.02830357 | 0.0753956 |
| 201688_s_at | TPD52 | −1.0610401 | 9.71085633 | −3.7945851 | 0.00035068 | 0.02857425 | 0.0640208 |
| 209960_at | HGF | −1.1259855 | 5.57784014 | −3.7926895 | 0.00035283 | 0.02870722 | 0.05844274 |
| 232232_s_at | SLC22A16 | −1.0425388 | 11.5170133 | −3.785579 | 0.00036104 | 0.02915779 | 0.03753396 |
| 223470_at | PIGM | −1.0448041 | 10.782124 | −3.7837211 | 0.00036321 | 0.02924694 | 0.03207456 |
| 218540_at | THTPA | −1.0678164 | 9.37118811 | −3.782058 | 0.00036517 | 0.02936123 | 0.0271889 |
| 226773_at | — | −1.0599901 | 8.73474937 | −3.7794255 | 0.00036829 | 0.02956841 | 0.0194579 |
| 238098_at | — | −1.1114847 | 5.09426112 | −3.778084 | 0.00036989 | 0.02961164 | 0.01551954 |
| 217707_x_at | SMARCA2 | −1.0808866 | 10.5195127 | −3.7780645 | 0.00036991 | 0.02961164 | 0.01546237 |
| 212993_at | — | −1.0777658 | 10.0764962 | −3.7708097 | 0.00037867 | 0.03004896 | −0.0058219 |
| 229123_at | — | −1.0812886 | 7.27161787 | −3.7693556 | 0.00038045 | 0.03009487 | −0.010085 |
| 235036_at | LIX1L | −1.0722631 | 8.44284176 | −3.7689888 | 0.0003809 | 0.03009487 | −0.0111601 |
| 1557261_at | WHDC1L1 // [symbol] | −1.1135936 | 8.0995202 | −3.7678412 | 0.00038231 | 0.03011937 | −0.0145238 |
| 228904_at | HOXB3 | −1.0881045 | 7.40156866 | −3.7669423 | 0.00038342 | 0.03016331 | −0.0171583 |
| 201381_x_at | CACYBP | −1.0345348 | 12.6646023 | −3.7613887 | 0.00039034 | 0.03049295 | −0.0334251 |
| 224800_at | WDFY1 | −1.0460265 | 11.2146548 | −3.7556441 | 0.00039763 | 0.03085591 | −0.0502365 |
| 228497_at | SLC22A15 | −1.0528433 | 10.2806063 | −3.7504565 | 0.00040432 | 0.03126767 | −0.0654047 |
| 218376_s_at | MICAL1 | −1.063991 | 10.8151846 | −3.7464229 | 0.0004096 | 0.0314976 | −0.0771901 |
| 214997_at | GOLGA1 | −1.1629895 | 5.40289373 | −3.7438471 | 0.000413 | 0.03166964 | −0.084712 |
| 232168_x_at | MACF1 | −1.1118803 | 7.19380131 | −3.7436429 | 0.00041327 | 0.03166964 | −0.0853084 |
| 215143_at | FLJ36166 | −1.1403943 | 5.25780477 | −3.7428445 | 0.00041434 | 0.03166964 | −0.0876391 |
| 229712_at | SNAPC3 | −1.1504135 | 5.48415471 | −3.7427571 | 0.00041445 | 0.03166964 | −0.0878942 |
| 208910_s_at | C1QBP | −1.0363862 | 12.212373 | −3.7403131 | 0.00041772 | 0.03172049 | −0.0950274 |
| 223343_at | MS4A7 | −1.0576569 | 11.3702217 | −3.7294854 | 0.00043249 | 0.03261559 | −0.1265955 |
| 216007_at | CLSTN2 | −1.1573199 | 5.8326347 | −3.7201011 | 0.00044569 | 0.03342695 | −0.153911 |
| 218477_at | TMEM14A | −1.0520488 | 11.0359972 | −3.7120317 | 0.00045735 | 0.03401174 | −0.1773659 |
| 225716_at | — | −1.0488569 | 12.276306 | −3.711697 | 0.00045784 | 0.03401174 | −0.1783381 |
| 229934_at | — | −1.1078549 | 9.5045234 | −3.7090958 | 0.00046167 | 0.03418201 | −0.1858919 |
| 226020_s_at | DAB1 /// O [symbol] | −1.0604674 | 9.19536151 | −3.7080794 | 0.00046317 | 0.03418201 | −0.1888427 |
| 231300_at | LOC90835 | −1.0706749 | 8.05881715 | −3.7080185 | 0.00046326 | 0.03418201 | −0.1890194 |
| 225431_x_at | ACY1L2 | −1.1051561 | 9.95149452 | −3.6951992 | 0.00048263 | 0.03517085 | −0.2261934 |
| 239503_at | — | −1.1019241 | 6.4612666 | −3.6931957 | 0.00048572 | 0.03517085 | −0.2319963 |
| 208747_s_at | C1S | −1.0897058 | 7.74085712 | −3.6813511 | 0.00050442 | 0.03616953 | −0.2662632 |
| 210658_s_at | GGA2 | −1.0464213 | 10.9126279 | −3.6763333 | 0.00051254 | 0.03648841 | −0.2807596 |
| 207405_s_at | RAD17 | −1.0614693 | 9.75893285 | −3.6739986 | 0.00051636 | 0.03662349 | −0.2875005 |
| 218792_s_at | BSPRY | −1.0964707 | 7.1685673 | −3.6700784 | 0.00052284 | 0.037029 | −0.2988132 |
| 224838_at | FOXP1 | −1.0451305 | 11.003461 | −3.666886 | 0.00052818 | 0.03716608 | −0.3080204 |
| 208956_x_at | DUT | −1.0305575 | 13.3123909 | −3.6657787 | 0.00053004 | 0.03717544 | −0.3112129 |
| 210375_at | PTGER3 | −1.122068 | 4.90394615 | −3.6657484 | 0.00053009 | 0.03717544 | −0.3113002 |
| 233252_s_at | STRBP | −1.0460631 | 9.42442497 | −3.6597179 | 0.00054034 | 0.03777915 | −0.3286758 |
| 202309_at | MTHFD1 | −1.0363875 | 11.5356701 | −3.6479618 | 0.00056088 | 0.03888847 | −0.3624982 |
| 212510_at | GPD1L | −1.0564567 | 9.5177254 | −3.6426294 | 0.00057044 | 0.03942724 | −0.3778178 |
| 232242_at | C3ORF14 | −1.1397197 | 5.05025398 | −3.6421706 | 0.00057127 | 0.03942724 | −0.3791352 |
| 203335_at | PHYH | −1.0509077 | 9.47404929 | −3.6414392 | 0.00057259 | 0.03942897 | −0.3812352 |
| 225876_at | NPAL3 | −1.0545782 | 8.47639608 | −3.6399342 | 0.00057533 | 0.03956752 | −0.3855556 |
| 226038_at | LONRF1 | −1.0686626 | 8.27265094 | −3.6331772 | 0.00058777 | 0.04027091 | −0.4049391 |
| 241466_at | MTL5 | −1.0881094 | 7.2321193 | −3.630577 | 0.00059262 | 0.04050197 | −0.4123921 |
| 232702_at | RABGAP1L | −1.1084819 | 7.2641058 | −3.629559 | 0.00059453 | 0.04053125 | −0.4153094 |
| 228157_at | ZNF207 | −1.1331898 | 9.20083267 | −3.628722 | 0.00059611 | 0.04058804 | −0.4177074 |
| 220892_s_at | PSAT1 | −1.0539762 | 10.5575882 | −3.6260997 | 0.00060107 | 0.04087498 | −0.4252181 |
| 211987_at | TOP2B | −1.0376579 | 11.6916878 | −3.6237021 | 0.00060564 | 0.04108363 | −0.4320824 |
| 207636_at | SERPINI2 | −1.1147369 | 8.32640727 | −3.6221365 | 0.00060864 | 0.04123615 | −0.4365631 |
| 1555702_a_a | ST3GAL3 | −1.0571721 | 11.469985 | −3.6202073 | 0.00061236 | 0.04138559 | −0.4420828 |
| 208967_s_at | AK2 | −1.0444004 | 11.8885466 | −3.6083697 | 0.00063567 | 0.04259196 | −0.4759123 |
| 227233_at | TSPAN2 | −1.0933291 | 4.88565746 | −3.6039803 | 0.00064452 | 0.04297454 | −0.488439 |
| 1561488_at | — | −1.0791431 | 8.42185259 | −3.5998639 | 0.00065293 | 0.04336219 | −0.5001777 |
| 232034_at | LOC203274 | −1.1061415 | 8.7194472 | −3.59871 | 0.00065531 | 0.04342891 | −0.5034667 |
| 210817_s_at | CALCOCO2 | −1.0381701 | 11.2547695 | −3.5973152 | 0.00065819 | 0.04351451 | −0.5074419 |
| 208990_s_at | HNRPH3 | −1.0433996 | 11.010468 | −3.5949274 | 0.00066315 | 0.04378974 | −0.5142443 |
| 205550_s_at | BRE | −1.042758 | 10.1098377 | −3.5935936 | 0.00066594 | 0.0438679 | −0.518043 |
| 218461_at | ATPBD1C | −1.044665 | 10.8560567 | −3.588935 | 0.00067577 | 0.04421781 | −0.5313034 |
| 203774_at | MTR | −1.0520755 | 1.5810662 | −3.5887762 | 0.00067611 | 0.04421781 | −0.5317553 |
| 1554791_a_a | FLJ23861 | −1.0802814 | 7.25343472 | −3.5862528 | 0.00068149 | 0.0444105 | −0.5389334 |
| 227662_at | SYNPO2 | −1.0748437 | 9.18363146 | −3.5850906 | 0.00068398 | 0.04451989 | −0.5422384 |
| 235374_at | MDH1 | −1.1308822 | 8.18431482 | −3.584254 | 0.00068578 | 0.04454185 | −0.5446169 |
| 211929_at | HNRPA3 | −1.0599635 | 11.1782682 | −3.5831072 | 0.00068826 | 0.04454185 | −0.5478769 |
| 236421_at | ANKRD45 | −1.1046422 | 7.81492792 | −3.5820889 | 0.00069046 | 0.04454185 | −0.5507713 |
| 222428_s_at | LARS | −1.044671 | 11.3066627 | −3.5819545 | 0.00069075 | 0.04454185 | −0.551153 |
| 218404_at | SNX10 | −1.0542239 | 10.5371703 | −3.5819241 | 0.00069082 | 0.04454185 | −0.5512396 |
| 225845_at | ZBTB44 | −1.0510504 | 10.114137 | −3.5792352 | 0.00069667 | 0.0448057 | −0.5588792 |
| 228030_at | — | −1.1453293 | 8.222179 | −3.5744486 | 0.00070722 | 0.04522467 | −0.5724699 |
| 205543_at | HSPA4L | −1.0619155 | 10.0871911 | −3.5711304 | 0.00071461 | 0.04550224 | −0.5818846 |
| 222786_at | CHST12 | −1.0953465 | 8.08550285 | −3.5710089 | 0.00071489 | 0.04550224 | −0.5822293 |
| 226148_at | ZBTB44 | −1.044973 | 10.9431965 | −3.5699976 | 0.00071716 | 0.04559364 | −0.5850975 |
| 220007_at | METTL8 | −1.0742738 | 7.97164029 | −3.568486 | 0.00072056 | 0.04570388 | −0.5893838 |
| 232049_at | — | −1.1055172 | 3.71843028 | −3.5666355 | 0.00072475 | 0.04531638 | −0.5946293 |
| 225639_at | SKAP2 | −1.0513915 | 10.9430745 | −3.5623166 | 0.00073462 | 0.04643409 | −0.6068655 |
| 201432_at | CAT | −1.029344 | 13.2389426 | −3.5605892 | 0.00073861 | 0.04663198 | −0.611757 |
| 243366_s_at | CERKL | −1.0677271 | 9.52062508 | −3.5590761 | 0.00074211 | 0.04665922 | −0.6160402 |
| 239163_at | UBE2B | −1.115315 | 5.28263677 | −3.5589302 | 0.00074245 | 0.04665922 | −0.6164534 |
| 239911_at | — | −1.1157309 | 6.92640942 | −3.5564148 | 0.00074832 | 0.04692001 | −0.6235711 |
| 239841_at | C7ORF24 | −1.1067027 | 8.40618863 | −3.5537484 | 0.00075458 | 0.04706777 | −0.6311131 |
| 213510_x_at | LOC220594 | −1.0787105 | 9.26871882 | −3.5532589 | 0.00075574 | 0.04706777 | −0.6324971 |
| 203446_s_at | OCRL | −1.0590145 | 8.52928391 | −3.5522168 | 0.00075821 | 0.04716146 | −0.6354436 |
| 220900_at | FLJ12078 | −1.2242895 | 4.04470354 | −3.550766 | 0.00076165 | 0.04732199 | −0.6395446 |
| 222427_s_at | LARS | −1.0327116 | 11.8350021 | −3.5464813 | 0.00077192 | 0.04764859 | −0.6516498 |
| 213908_at | WHDC1L1 // [symbol] | −1.106503 | 7.49688229 | −3.54639 | 0.00077214 | 0.04764859 | −0.6519076 |
| 235170_at | ZNF92 | −1.0701713 | 10.4753161 | −3.5440334 | 0.00077784 | 0.04781967 | −0.6585616 |
| 205804_s_at | TRAF3IP3 | −1.0645947 | 10.430865 | −3.5397143 | 0.0007884 | 0.04837904 | −0.6707492 |
| 227865_at | C9ORF103 | −1.0549476 | 9.09854389 | −3.537566 | 0.0007937 | 0.04864948 | −0.6768078 |
| 201769_at | CLINT1 | −1.0404695 | 11.3472898 | −3.5358759 | 0.0007979 | 0.04872219 | −0.6815728 |
| 1555962_at | B3GNT7 | −1.0816315 | 8.07026497 | −3.5328566 | 0.00080544 | 0.04891403 | −0.6900812 |
| 244050_at | PTPLAD2 | −1.0466927 | 10.2444943 | −3.5293148 | 0.00081438 | 0.04930933 | −0.7000563 |
| 1554704_at | ATP8B3 | −1.0775601 | 9.03671576 | −3.5253989 | 0.00082438 | 0.04963954 | −0.7110776 |

[symbol]

indicates data missing or illegible when filed

### Polimunol and Copaxone Comparison: Differentially Expressed Pathways

In order to identify pathways differentially expressed by Polimunol compared with Copaxone, the input probesets had to comply not only with the conservative steps described above (resulting in a set of 779 differentially expressed probesets), but also with a fold change threshold of |FC|≧1.1. Pathway enrichment analysis employed the NIH DAVID platform, resulting in 137 pathways enriched among top probesets upregulated by Polimunol relative to Copaxone®. Pathways significantly enriched among top probesets differentially expressed between Polimunol and Copaxone in THP-1 cells are listed in Table 29.

TABLE 29
Pathways significantly enriched among top probesets
differentially expressed between Polimunol and Copaxone ® in THP-1 cells.

| | | | | Fold | |
|---|---|---|---|---|---|
| Category | Term | Count | Pvalue | Enrichment | Benjamini |
| GOTERM_BP_ALL | GO: G009605~response to external stimulus | 34 | 125E−12 | 4.242127482 | 2.13E−09 |
| GOTERM_BP_ALL | GO: 0050896~response to stimulus | 66 | 1.47E−11 | 2.221777115 | 1.25E−08 |
| GOTERM_BP_ALL | GO: 0009611~response to wounding | 24 | 1.10E−10 | 5.226859162 | 6.26E−08 |
| GOTERM_BP_ALL | GO: 0051239~regulation of multicellular organismal | 30 | 2.86E−09 | 3.546434111 | 1.22E−06 |
| | process | | | | |
| GOTERM_BP_ALL | GO: 0006950~response to stress | 45 | 3.97E−09 | 2.52009281 | 1.35E−06 |
| GOTERM_CC_ALL | GO: 0005576~extracellular region | 32 | 1.62E−08 | 3.124422358 | 2.46E−06 |
| GOTERM_BP_ALL | GO: 0002376~immune system process | 31 | 9.83E−09 | 3.264522306 | 2.79E−06 |
| GOTERM_MF_ALL | GO: 0004888~transmembrane receptor activity | 21 | 7.19E−08 | 4.277319182 | 9.08E−06 |
| GOTERM_MF_ALL | GO: 0004872~receptor activity | 30 | 6.09E−08 | 3.099082935 | 1.15E−05 |
| GOTERM_BP_ALL | GO: 0006955~immune response | 23 | 6.20E−08 | 3.925650193 | 1.51E−05 |
| KEGG_PATHWAY | hsa04060: Cytokine-cytokine receptor interaction | 14 | 2.08E−07 | 6.068719076 | 1.92E−05 |
| GOTERM_MF_ALL | GO: 0004871~signal transducer activity | 37 | 5.51E−08 | 2.648089319 | 2.09E−05 |
| GOTERM_MF_ALL | GO: 0060089~molecular transducer activity | 37 | 5.51E−08 | 2.648089319 | 2.09E−05 |
| GOTERM_BP_ALL | GO: 0006952~defense response | 21 | 1.12E−07 | 4.15637457 | 2.37E−05 |
| GOTERM_BP_ALL | GO: 0032879~regulation of localization | 22 | 1.58E−07 | 3.885957066 | 2.98E−05 |
| GOTERM_MF_ALL | GO: 0030247~polysaccharide binding | 10 | 4.26E−07 | 10.28108466 | 4.04E−05 |
| GOTERM_MF_ALL | GO: 0001871~pattern binding | 10 | 4.26E−07 | 10.28108466 | 4.04E−05 |
| GOTERM_CC_ALL | GO: 0005615~extracellular space | 17 | 6.80E−07 | 4.626388691 | 5.17E−05 |
| GOTERM_BP_ALL | GO: 0048583~regulation of response to stimulus | 19 | 3.08E−07 | 4.32390456 | 5.23E−05 |
| GOTERM_CC_ALL | GO: 0044421~extracellular region part | 19 | 2.56E−06 | 3.750331428 | 9.73E−05 |
| GOTERM_CC_ALL | GO: 0005886~plasma membrane | 51 | 1.99E−06 | 1.918590604 | 0.00010059 |
| GOTERM_BP_ALL | GO: 0050794~regulation of cellular process | 97 | 7.60E−07 | 1.431262036 | 0.00010778 |
| GOTERM_BP_ALL | GO: 0042127~regulation of cell proliferation | 25 | 8.88E−07 | 3.138545339 | 0.00010788 |
| GOTERM_BP_ALL | GO: 0007166~cell surface receptor linked signal | 30 | 8.25E−07 | 2.737037737 | 0.000108 |
| | transduction | | | | |
| GOTERM_BP_ALL | GO: 0006954~inflammatory response | 15 | 7.41E−07 | 5.299032363 | 0.0001146 |
| GOTERM_BP_ALL | GO: 0042221~response to chemical stimulus | 33 | 1.27E−06 | 2.509579282 | 0.00013487 |
| GOTERM_BP_ALL | GO: 0050789~regulation of biological process | 99 | 1.24E−06 | 1.405133361 | 0.00014066 |
| GOTERM_BP_ALL | GO: 0065007~biological regulation | 101 | 2.61E−06 | 1.374157191 | 0.00023327 |
| GOTERM_BP_ALL | GO: 0048584~positive regulation of response to stimulus | 13 | 2.38E−06 | 5.72397892 | 0.00023849 |
| GOTERM_BP_ALL | GO: 0002684~positive regulation of immune system | 13 | 2.57E−06 | 5.682799215 | 0.00024311 |
| | process | | | | |
| GOTERM_BP_ALL | GO: 0001568~blood vessel development | 13 | 4.31E−06 | 5.410336239 | 0.00036654 |
| GOTERM_BP_ALL | GO: 0001944~vasculature development | 13 | 4.97E−06 | 5.337223587 | 0.00038399 |
| GOTERM_BP_ALL | GO: 0042060~wound healing | 11 | 4.89E−06 | 6.683846154 | 0.00039593 |
| GOTERM_BP_ALL | GO: 0048519~negative regulation of biological process | 41 | 6.21E−06 | 2.055488241 | 0.00040825 |
| GOTERM_BP_ALL | GO: 0048514~blood vessel morphogenesis | 12 | 6.15E−06 | 5.786879787 | 0.00041817 |
| GOTERM_BP_ALL | GO: 0002682~regulation of immune system process | 16 | 5.94E−06 | 4.154682924 | 0.00042096 |
| GOTERM_BP_ALL | GO: 0007165~signal transduction | 45 | 5.71E−06 | 1.96288636 | 0.00042219 |
| GOTERM_BP_ALL | GO: 0050793~regulation of developmental process | 21 | 7.97E−06 | 3.166270454 | 0.0004676 |
| GOTERM_BP_ALL | GO: 0048731~system development | 41 | 7.80E−06 | 2.037000612 | 0.00047369 |
| GOTERM_BP_ALL | GO: 0050776~regulation of immune response | 12 | 7.72E−06 | 5.652301187 | 0.00048624 |
| GOTERM_BP_ALL | GO: 0051240~positive regulation of multicellular | 12 | 1.19E−05 | 5.401087801 | 0.0006771 |
| | organismal process | | | | |
| GOTERM_BP_ALL | GO: 0060341~regulation of cellular localization | 11 | 2.31E−05 | 5.61667744 | 0.00126569 |
| GOTERM_BP_ALL | GO: 0032501~multicellular organismal process | 55 | 2.47E−05 | 1.69297015 | 0.00131208 |
| GOTERM_BP_ALL | GO: 0002703~regulation of leukocyte mediated immunity | 7 | 3.08E−05 | 11.1930438 | 0.00158406 |
| GOTERM_MF_ALL | GO: 0005539~glycosaminoglycan binding | 8 | 2.69E−05 | 8.933908046 | 0.0020367 |
| GOTERM_BP_ALL | GO: 0048646~anatomical structure formation involved in | 14 | 5.46E−05 | 3.902162058 | 0.00272943 |
| | morphogenesis | | | | |
| GOTERM_BP_ALL | GO: 0043066~negative regulation of apoptosis | 15 | 8.17E−05 | 3.519048519 | 0.00396163 |
| GOTERM_MF_ALL | GO: 0001608~nucleotide receptor activity, G-protein | 5 | 6.34E−05 | 21.59027778 | 0.00399802 |
| | coupled | | | | |
| GOTERM_MF_ALL | GO: 0045028~purinergic nucleotide receptor activity, G- | 5 | 6.34E−05 | 21.59027778 | 0.00399802 |
| | protein coupled | | | | |
| GOTERM_BP_ALL | GO: 0043069~negative regulation of programmed cell | 15 | 9.24E−05 | 3.47875407 | 0.00423954 |
| | death | | | | |
| KEGG_PATHWAY | hsa04610: Complement and coagulation cascades | 6 | 9.31E−05 | 12.09974425 | 0.00427423 |
| GOTERM_BP_ALL | GO: 0060548~negative regulation of cell death | 15 | 9.63E−05 | 3.465526869 | 0.00429948 |
| GOTERM_BP_ALL | GO: 0048856~anatomical structure development | 41 | 9.18E−05 | 1.834500551 | 0.00433037 |
| GOTERM_BP_ALL | GO: 0001525~angiogenesis | 9 | 0.00010467 | 6.076223776 | 0.00444135 |
| GOTERM_BP_ALL | GO: 0001819~positive regulation of cytokine production | 8 | 0.00010299 | 7.255192569 | 0.00448212 |
| GOTERM_BP_ALL | GO: 0048660~regulation of smooth muscle cell | 6 | 0.00011139 | 12.15244755 | 0.00461078 |
| | proliferation | | | | |
| GOTERM_BP_ALL | GO: 0048518~positive regulation of biological process | 40 | 0.00011815 | 1.834331706 | 0.00477377 |
| GOTERM_BP_ALL | GO: 0032502~developmental process | 48 | 0.00013028 | 1.687839938 | 0.00514072 |
| GOTERM_BP_ALL | GO: 0007275~multicellular organismal development | 44 | 0.00014296 | 1.743991169 | 0.00551191 |
| GOTERM_BP_ALL | GO: 0010033~response to organic substance | 20 | 0.00014822 | 2.665010428 | 0.00558755 |
| GOTERM_BP_ALL | GO: 0009617~response to bacterium | 9 | 0.00015325 | 5.756422525 | 0.0056515 |
| GOTERM_MF_ALL | GO: 0016502~nucleotide receptor activity | 5 | 0.00010797 | 19.0502451 | 0.00582884 |
| GOTERM_MF_ALL | GO: 0001614~purinergic nucleotide receptor activity | 5 | 0.00010797 | 19.0502451 | 0.00582884 |
| GOTERM_BP_ALL | GO: 0032496~response to lipopolysaccharide | 7 | 0.00016853 | 8.339914987 | 0.00608127 |
| GOTERM_BP_ALL | GO: 0045765~regulation of angiogenesis | 6 | 0.00017834 | 11.04767959 | 0.00630065 |
| GOTERM_CC_ALL | GO: 0031226~intrinsic to plasma membrane | 21 | 0.00020851 | 2.519724996 | 0.00631924 |
| GOTERM_BP_ALL | GO: 0048513~organ development | 31 | 0.00018295 | 2.027588128 | 0.00633147 |
| GOTERM_BP_ALL | GO: 0051707~response to other organism | 11 | 0.00021593 | 4.312158809 | 0.00731991 |
| GOTERM_BP_ALL | GO: 0051270~regulation of cell motion | 10 | 0.00022302 | 4.78442817 | 0.00741176 |
| GOTERM_BP_ALL | GO: 0031099~regeneration | 6 | 0.00023762 | 10.41638362 | 0.00774377 |
| GOTERM_MF_ALL | GO: 0030246~carbohydrate binding | 11 | 0.00017696 | 4.425336439 | 0.00834919 |
| GOTERM_CC_ALL | GO: 0005887~integral to plasma membrane | 20 | 0.00047736 | 2.436305529 | 0.01031444 |
| GOTERM_BP_ALL | GO: 0030334~regulation of cell migration | 9 | 0.00034868 | 5.110842429 | 0.01092533 |
| GOTERM_BP_ALL | GO: 0051049~regulation of transport | 13 | 0.00034269 | 3.464513557 | 0.01094009 |
| GOTERM_BP_ALL | GO: 0002237~response to molecule of bacterial origin | 7 | 0.00037883 | 7.209079057 | 0.01165013 |
| GOTERM_BP_ALL | GO: 0007186~G-protein coupled receptor protein | 13 | 0.00040141 | 3.404780564 | 0.01170574 |
| | signaling pathway | | | | |
| GOTERM_BP_ALL | GO: 0002697~regulation of immune effector process | 7 | 0.00041534 | 7.088927739 | 0.01170818 |
| GOTERM_CC_ALL | GO: 0016020~membrane | 82 | 0.00046587 | 1.342587724 | 0.01173539 |
| GOTERM_BP_ALL | GO: 0002822~regulation of adaptive immune response | 6 | 0.00039991 | 9.348036579 | 0.01186565 |
| | based on somatic recombination of immune receptors | | | | |
| | built from immunoglobulin superfamily domains | | | | |
| GOTERM_BP_ALL | GO: 0009968~negative regulation of signal transduction | 10 | 0.00041443 | 4.403060707 | 0.01187952 |
| GOTERM_BP_ALL | GO: 0044057~regulation of system process | 10 | 0.00041443 | 4.403060707 | 0.01187952 |
| GOTERM_BP_ALL | GO: 0051046~regulation of secretion | 8 | 0.00039574 | 5.85660123 | 0.01195094 |
| GOTERM_BP_ALL | GO: 0050778~positive regulation of immune response | 8 | 0.00039574 | 5.85660123 | 0.01195094 |
| GOTERM_BP_ALL | GO: 0006935~chemotaxis | 8 | 0.00045788 | 5.718798848 | 0.01248643 |
| GOTERM_BP_ALL | GO: 0042330~taxis | 8 | 0.00045788 | 5.718798848 | 0.01248643 |
| GOTERM_BP_ALL | GO: 0002819~regulation of adaptive immune response | 6 | 0.0004511 | 9.114335664 | 0.01250293 |
| GOTERM_BP_ALL | GO: 0051101~regulation of DNA binding | 8 | 0.00049175 | 5.652301187 | 0.01319259 |
| GOTERM_BP_ALL | GO: 0048523~negative regulation of cellular process | 34 | 0.00050059 | 1.834738973 | 0.01321981 |
| GOTERM_BP_ALL | GO: 0009653~anatomical structure morphogenesis | 24 | 0.00055741 | 2.16043512 | 0.01448509 |
| GOTERM_BP_ALL | GO: 0080134~regulation of response to stress | 11 | 0.00057031 | 3.819340659 | 0.01459518 |
| GOTERM_MF_ALL | GO: 0019955~cytokine binding | 7 | 0.00035486 | 7.312836022 | 0.01483488 |
| GOTERM_BP_ALL | GO: 0042981~regulation of apoptosis | 22 | 0.00060362 | 2.254248281 | 0.01521241 |
| GOTERM_CC_ALL | GO: 0031224~intrinsic to membrane | 60 | 0.00085774 | 1.452927379 | 0.01617187 |
| GOTERM_BP_ALL | GO: 0040012~regulation of locomotion | 9 | 0.00067325 | 4.634407965 | 0.01670569 |
| GOTERM_BP_ALL | GO: 0043067~regulation of programmed cell death | 22 | 0.00068835 | 2.231668165 | 0.0168318 |
| GOTERM_BP_ALL | GO: 0010941~regulation of cell death | 22 | 0.0007188 | 2.224241649 | 0.01732135 |
| GOTERM_BP_ALL | GO: 0010648~negative regulation of cell communication | 10 | 0.00076041 | 4.050815851 | 0.01781109 |
| GOTERM_BP_ALL | GO: 0001817~regulation of cytokine production | 9 | 0.00075248 | 4.557167832 | 0.01787284 |
| GOTERM_BP_ALL | GO: 0030154~cell differentiation | 28 | 0.00084605 | 1.942171983 | 0.01952917 |
| GOTERM_BP_ALL | GO: 0002460~adaptive immune response based on | 6 | 0.00086878 | 7.925509273 | 0.01978057 |
| | somatic recombination of immune receptors built from | | | | |
| | immunoglobulin superfamily domains | | | | |
| GOTERM_BP_ALL | GO: 0002250~adaptive immune response | 6 | 0.00086878 | 7.925509273 | 0.01978057 |
| GOTERM_BP_ALL | GO: 0065008~regulation of biological quality | 27 | 0.00088955 | 1.969484297 | 0.01998171 |
| GOTERM_BP_ALL | GO: 0001775~cell activation | 11 | 0.0009539 | 3.57424928 | 0.02059711 |
| GOTERM_BP_ALL | GO: 0051223~regulation of protein transport | 7 | 0.00095182 | 6.076223776 | 0.0208168 |
| GOTERM_BP_ALL | GO: 0048869~cellular developmental process | 29 | 0.00094798 | 1.89677599 | 0.02100365 |
| KEGG_PATHWAY | hsa04621: NOD-like receptor signaling pathway | 7 | 0.00076766 | 6.125971143 | 0.02327548 |
| GOTERM_CC_ALL | GO: 0033256~I-kappaB/NF-kappaB complex | 3 | 0.00140853 | 47.9647651 | 0.02352421 |
| GOTERM_BP_ALL | GO: 0002694~regulation of leukocyte activation | 8 | 0.0011427 | 4.910079819 | 0.02431771 |
| GOTERM_BP_ALL | GO: 0050817~coagulation | 6 | 0.00116213 | 7.440274012 | 0.02442105 |
| GOTERM_BP_ALL | GO: 0007596~blood coagulation | 6 | 0.00116213 | 7.440274012 | 0.02442105 |
| GOTERM_BP_ALL | GO: 0006916~anti-apoptosis | 10 | 0.00120323 | 3.79763986 | 0.02466543 |
| GOTERM_BP_ALL | GO: 0009607~response to biotic stimulus | 12 | 0.00120013 | 3.212100675 | 0.02490264 |
| GOTERM_BP_ALL | GO: 0008285~negative regulation of cell proliferation | 12 | 0.0012436 | 3.198012514 | 0.02517972 |
| GOTERM_MF_ALL | GO: 0004930~G-protein coupled receptor activity | 9 | 0.00068792 | 4.626488095 | 0.02574405 |
| GOTERM_CC_ALL | GO: 0009986~cell surface | 10 | 0.001793 | 3.5928663 | 0.02690939 |
| GOTERM_BP_ALL | GO: 0014070~response to organic cyclic substance | 7 | 0.00136752 | 5.671142191 | 0.02733082 |
| GOTERM_BP_ALL | GO: 0040011~locomotion | 12 | 0.00148086 | 3.129385636 | 0.02888628 |
| GOTERM_BP_ALL | GO: 0070201~regulation of establishment of protein | 7 | 0.0014651 | 5.596521899 | 0.02891444 |
| | localization | | | | |
| GOTERM_BP_ALL | GO: 0046649~lymphocyte activation | 9 | 0.00154141 | 4.08104582 | 0.02971013 |
| GOTERM_BP_ALL | GO: 0031328~positive regulation of cellular biosynthetic | 17 | 0.00162069 | 2.413453369 | 0.03052477 |
| | process | | | | |
| GOTERM_BP_ALL | GO: 0050865~regulation of cell activation | 8 | 0.00161108 | 4.62950383 | 0.03068591 |
| GOTERM_BP_ALL | GO: 0007626~locomotory behavior | 9 | 0.00169408 | 4.02103044 | 0.03119615 |
| GOTERM_BP_ALL | GO: 0070304~positive regulation of stress-activated | 4 | 0.0016835 | 16.2032634 | 0.03134323 |
| | protein kinase signaling pathway | | | | |
| GOTERM_BP_ALL | GO: 0007599~hemostasis | 6 | 0.00180567 | 6.751359751 | 0.03286331 |
| GOTERM_BP_ALL | GO: 0002706~regulation of lymphocyte mediated | 5 | 0.00193607 | 9.206399661 | 0.03482477 |
| | immunity | | | | |
| GOTERM_BP_ALL | GO: 0009891~positive regulation of biosynthetic process | 17 | 0.00195922 | 2.369169821 | 0.03486599 |
| GOTERM_BP_ALL | GO: 0008284~positive regulation of cell proliferation | 12 | 0.00206731 | 3.000604334 | 0.03637597 |
| GOTERM_BP_ALL | GO: 0048522~positive regulation of cellular process | 34 | 0.00209766 | 1.68922002 | 0.03652324 |
| GOTERM_BP_ALL | GO: 0043433~negative regulation of transcription factor | 5 | 0.00216669 | 8.9356232 | 0.03732224 |
| | activity | | | | |
| GOTERM_BP_ALL | GO: 0051098~regulation of binding | 8 | 0.00221714 | 4.379260379 | 0.03741874 |
| GOTERM_BP_ALL | GO: 0010646~regulation of cell communication | 22 | 0.00220111 | 2.034656363 | 0.0375249 |
| GOTERM_BP_ALL | GO: 0048568~embryonic organ development | 7 | 0.0023046 | 5.124526076 | 0.03848637 |
| GOTERM_MF_ALL | GO: 0004896~cytokine receptor activity | 5 | 0.0013647 | 10.12044271 | 0.04596231 |
| GOTERM_BP_ALL | GO: 0045944~positive regulation of transcription from | 11 | 0.00298615 | 3.065984474 | 0.04864968 |
| | RNA polymerase II promoter | | | | |
| GOTERM_BP_ALL | GO: 0043392~negative regulation of DNA binding | 5 | 0.00297111 | 8.211113211 | 0.04887781 |
| GOTERM_CC_ALL | GO: 0016021~integral to membrane | 56 | 0.00368755 | 1.398972315 | 0.04976835 |
| GOTERM_BP_ALL | GO: 0032880~regulation of protein localization | 7 | 0.00309631 | 4.833359822 | 0.04992419 |

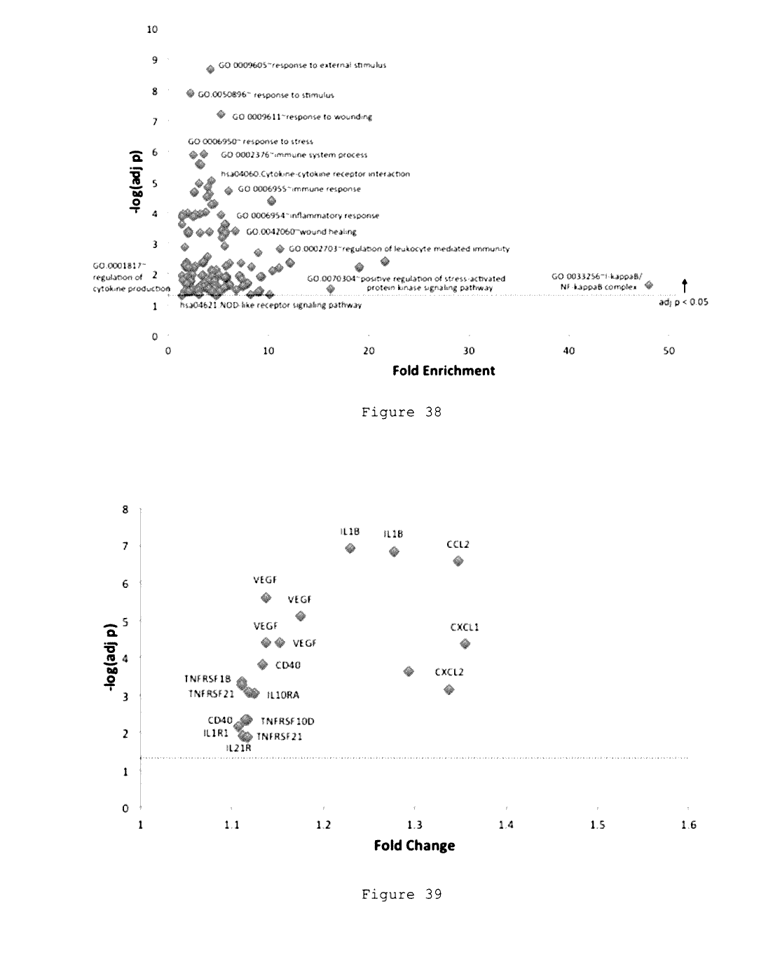

These pathways include a variety of immune related pathways expected to be relevant to Copaxone's mechanism of action, including immune-related pathways such as the cytokine-cytokine receptor interaction pathway (adjusted p<1.9e-5) that was also enriched among probesets modulated by Copaxone® (see above), and positive regulation of cytokine production (adjusted p<0.004). These pathways also include inflammation related pathways, such as inflammatory response (adjusted p<0.0001), NOD-like receptor signaling (adjusted p<0.02), and response to lipopolysaccharide (adjusted p<0.006). The top 25 pathways are listed in Table 30, and the key pathways are illustrated in FIG. 38. The specific genes in the cytokine-cytokine receptor interaction pathway are depicted in FIG. 39.

TABLE 30
Top 25 pathways significantly enriched among top probesets
differentially expressed between Polimunol and Copaxone

| | | | Adjusted |
|---|---|---|---|
| Category | Term | Count | p value |
| GO Biological Process | GO: 0009605~response to external | 34 | 2.13E−09 |
| | stimulus | | |
| GO Biological Process | GO: 0050896~response to stimulus | 66 | 1.25E−08 |
| GO Biological Process | GO: 0009611~response to wounding | 24 | 6.26E−08 |
| GO Biological Process | GO: 0051239~regulation of | 30 | 1.22E−06 |
| | multicellular organismal process | | |
| GO Biological Process | GO: 0006950~response to stress | 45 | 1.35E−06 |
| GO Cellular | GO:0005576~extracellular region | 32 | 2.46E−06 |
| Compartment | | | |
| GO Biological Process | GO: 0002376~immune system | 31 | 2.79E−06 |
| | process | | |
| GO Molecular Function | GO: 0004888~transmembrane | 21 | 9.08E−06 |
| | receptor activity | | |
| GO Molecular Function | GO: 0004872~receptor activity | 30 | 1.15E−05 |
| GO Biological Process | GO: 0006955~immune response | 23 | 1.51E−05 |
| Kegg Pathway | hsa04060: Cytokine-cytokine | 14 | 1.92E−05 |
| | receptor interaction | | |
| GO Molecular Function | GO: 0004871~signal transducer | 37 | 2.09E−05 |
| | activity | | |
| GO Molecular Function | GO: 0060089~molecular transducer | 37 | 2.09E−05 |
| | activity | | |
| GO Biological Process | GO: 0006952~defense response | 21 | 2.37E−05 |
| GO Biological Process | GO: 0032879~regulation of | 22 | 2.98E−05 |
| | localization | | |
| GO Molecular Function | GO: 0030247~polysaccharide | 10 | 4.04E−05 |
| | binding | | |
| GO Molecular Function | GO: 0001871~pattern binding | 10 | 4.04E−05 |
| GO Cellular | GO: 0005615~extracellular space | 17 | 5.17E−05 |
| Compartment | | | |
| GO Biological Process | GO: 0048583~regulation of response | 19 | 5.23E−05 |
| | to stimulus | | |
| GO Cellular | GO: 0044421~extracellular region | 19 | 9.73E−05 |
| Compartment | part | | |
| GO Cellular | GO: 0005886~plasma membrane | 51 | 0.00010059 |
| Compartment | | | |
| GO Biological Process | GO: 0050794~regulation of cellular | 97 | 0.000107777 |
| | process | | |
| GO Biological Process | GO: 0042127~regulation of cell | 25 | 0.000107884 |
| | proliferation | | |
| GO Biological Process | GO: 0007166~cell surface receptor | 30 | 0.000108003 |
| | linked signal transduction | | |
| GO Biological Process | GO: 0006954~inflammatory | 15 | 0.000114601 |
| | response | | |

### Polimunol and Copaxone Comparison: Discussion

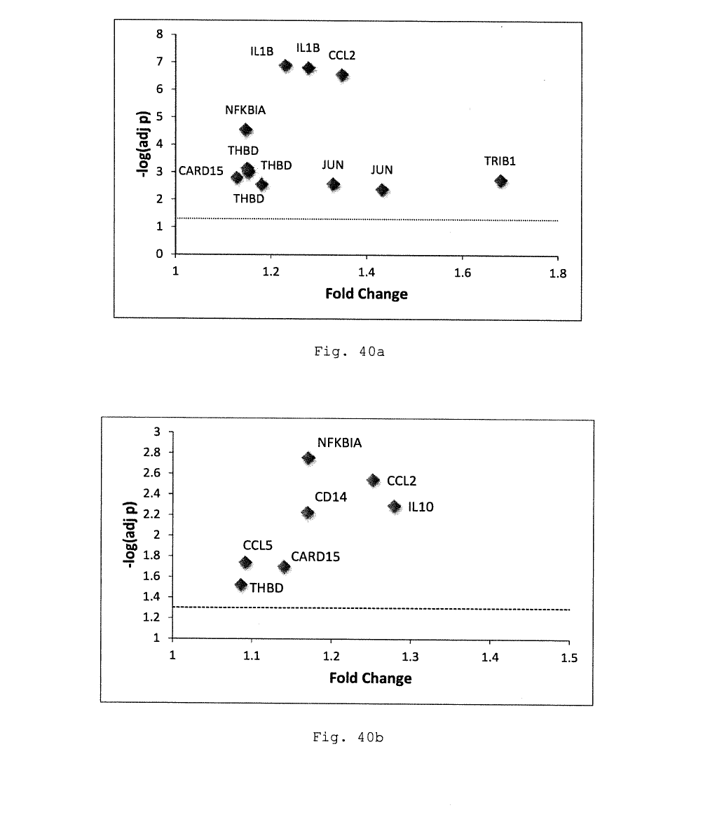

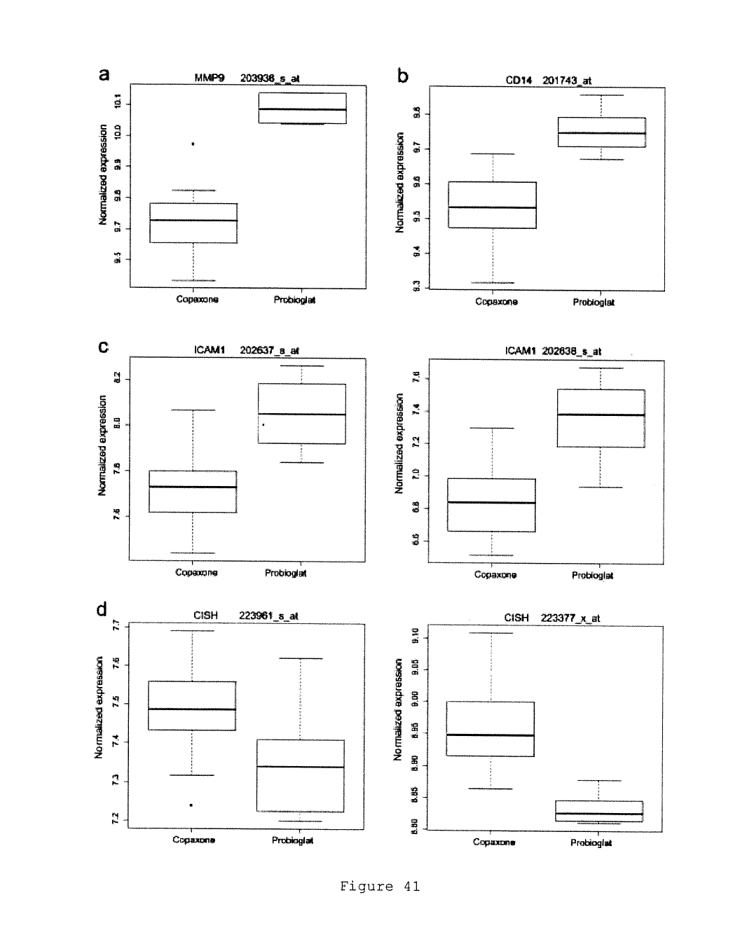

Once demonstrated to be robustly designed as shown in the MoA sub-section, and upon applying conservative statistical approaches, hundreds of genes are differentially expressed in human monocytes following activation with Polimunol compared to activation with Copaxone®. In and of itself, the fact that so many genes are differentially expressed immediately calls into question the “sameness” of Polimunol and suggests that its biological impact on human antigen presenting cells differs significantly from Copaxone®. This stands in sharp contrast to the lack of differences between the three different lots of Copaxone tested in parallel, under blinding, in the same experiment (in the three possible pairwise comparisons between Copaxone® lots, for two of the comparisons zero significant probesets were observed, and for the third a single probeset passed FDR with adjusted p value of 0.044). Moreover, the fact that the differentially expressed genes are enriched in key biological pathways such as “immune response” further supports the biological relevance of the observed differences. Finally, the fact that many of the pathways enriched among the differentially expressed genes are relevant to Copaxone's mechanism of action, such as cytokine-cytokine receptor interactions, and relevant to potential safety concerns, such as inflammatory response and response to lipopolysaccharide pathways, together raise serious concerns regarding the safety and efficacy of Polimunol. In fact, the response to lipopolysaccharide pathway was also enriched among genes differentially expressed by another purported generic, Probioglat (FIG. 40). Out of the 137 pathways enriched among probesets upregulated by Polimunol relative to Copaxone®, 37 of these pathways were also enriched in the prior comparison with Probioglat (out of 64 total pathways seen for Probioglat). Of note, the introduction of Probioglat in Mexico was associated with a 3-fold increase in adverse events and a 7-fold increase in relapses as detailed in Teva's seventh Citizen Petition (FDA-2014-P-0933-0001 available at www.regulations.gov/#?documentDetail;D=FDA-2014-P-0933-0001, and also described herein).

### Discussion

### Example 10-11

Copaxone® has long provided a safe and effective treatment option for multiple sclerosis patients, operating through a complex set of mechanisms that have gradually been elucidated through extensive research that continues to this day.

Methods for comparing two small molecule medicines to determine therapeutic equivalence are well established, and standards for evaluating biologics are also rapidly becoming available. However, for non-biological complex drugs (NBCDs) such as glatiramer acetate the level of evidence needed to determine equivalence remains an unresolved question and a subject of ongoing research by the scientific community. Teva is an active participant in such research, conducting a steady program of experiments to characterize Copaxone® and compare it to various glatiramoids marketed outside the United States and Europe as purported generics. Such characterization studies are critical to ensure that patients with multiple sclerosis (MS) continue to receive adequate medicines that are efficacious and safe.

One key aspect of the public discourse on non-biological complex drugs concerns the role of clinical trials. Well-controlled clinical trials of appropriate duration (typically 2 years) using standard endpoints such as annualized relapse rate (ARR) are an important means for investigating the safety and efficacy of medicines for MS, yet clinical studies not meeting those criteria distract from the essential question of “sameness” and risk creating a false sense of security for a purported generic. In recent months, Synthon has publicly reported clinical results for a follow-on glatiramoid purported to be a generic form of Copaxone, yet the trial results raise significant questions regarding its validity [as detailed in Appendix 2 of FDA-2014-P-0933-0001 available at www.regulations.gov/#?documentDetail;D=FDA-2014-P-0933-0001, and also described herein]. For instance, given the sample size of GATE (Copaxone=357, Placebo=84), the probability of showing any level of annualized relapse rate (ARR) reduction (i.e. clinical efficacy) in the reference, well-established drug Copaxone®, using results from prior adequate, randomized, placebo-controlled studies with Copaxone® was high (>90%). Yet, a statistically unlikely result of no effect in reducing ARR was recorded for the Copaxone® arm in the GATE study, attesting to the lack of assay sensitivity. It is critical to know whether the trial had assay sensitivity (i.e., could have distinguished an effective drug from an ineffective one). Thus, if the active control (Copaxone® in the case of GATE study) had no effect at all in the trial (i.e., did not have any of its well-established, expected effect), then finding even a very small difference between control and test drug is meaningless, providing no evidence that the test drug is effective. Lastly, the rate of adverse events leading to discontinuation was 2.4 fold higher in the Synthon product arm than in the Copaxone® arm (12 vs 5). Overall, the many shortcomings of the design and conduct of the GATE study render its findings unreliable.

Teva is continuously conducting experiments to further elucidate Copaxone®'s complex mechanism of action, and working towards the discovery of validated biomarkers, which have yet to be identified. Teva then seeks to compare Copaxone®'s profile to that of all purported generics marketed globally. Synthon's Polimunol was recently introduced to clinical practice in Argentina, and has since been studied by Teva extensively through a variety of characterization methods, each providing a small window into specific characteristics of this complex medicine. For instance, genome-wide transcriptional studies were employed in carefully selected model systems informative of certain aspects of the biological impact of an immunological medicine. To compare the ex-vivo biological effect of Copaxone® and Polimunol in mouse lymphocytes following prior in vivo immunization, mouse splenocytes were utilized, which embody the key cell type impacted by antigenic presentation. Additionally, to compare the immediate biological effects of Copaxone® and Polimunol in antigen-presenting cells, which are key to the mode of action of an antigen in mediating T-helper cell immune response, a human monocyte (THP-1) cell line was utilized. In both model systems significant differences in gene expression profiles were detected between Copaxone and Polimunol, regardless of the immunization sequence or array randomization scheme.

In mouse splenocytes, the fact that significant and consistent differences in gene expression profiles are observed between splenocytes ex-vivo activated with Polimunol and splenocytes activated with Copaxone®, regardless of whether the mice were initially in vivo immunized with Copaxone or initially immunized with Polimunol, further emphasizes the robustness of the differences in biological impact of these two medicines on many genes. These hundreds of affected genes participate in immunologically relevant pathways and highlight potential clinical implications of overly stimulated or under-regulated immunological mechanisms relevant to the response and well-being of MS patients. For instance, IL18, which has been implicated in the pathogenesis of MS, is downregulated significantly less effectively by Polimunol than by Copaxone. IL18 is important for T helper cell differentiation, and influences IFNg expression, suggesting that the impact of each medicine on T cells (which play an important role in Copaxone's mechanism of action) is significantly different. The importance of IL18 via its actions in inducing Th1 responses (which is attributed, at least partially, to induction of IFNg production by natural killer (NK) cells), was underscored in a study in the mouse model of MS, autoimmune encephalomyelitis (EAE). Mice deficient for IL18 were observed to be resistant to EAE, and treatment of these mice with IL18 restored the ability to generate a Th1 response, while treatment of mice wild-type for IL18 increased EAE disease severity (Shi et al, J. Immunol., 2000). IL18 antibodies as well as the naturally expressed IL18 inhibitor, IL18BP (which was upregulated by Copaxone treatment in this study), have been proposed as potential therapeutic agents against neuroinflammation (Felderhoff-Mueser et al, Trends Neurosci, 2005).

Similarly, key type I interferon pathway production pathway genes including RIG-I, MDA5, and IRF7 are all significantly upregulated by Polimunol relative to Copaxone®. This increased interferon signaling with Polimunol treatment further increases concerns about possible adverse events including flu-like symptoms in Polimunol-treated patients. As noted in Trinchieri et al, 2010, “IFN produced during viral infection, other pathological conditions, or in the presence of DNA released by dying cells may mediate unwanted toxicity or induce pathological damage and inflammatory or autoimmune syndromes (Pascual et al., 2010).”

In human monocytes, many of the pathways enriched among the genes differentially expressed between Copaxone and Polimunol are relevant to Copaxone's mechanism of action (such as cytokine-cytokine receptor interaction), and relevant to potential safety concerns (such as inflammatory response and response to lipopolysaccharide pathways). The same response to lipopolysaccharide pathway was also enriched among genes differentially expressed by another purported generic, Probioglat. This is particularly concerning because the introduction of another purported generic to Copaxone, Probioglat in Mexico was associated with a 3-fold increase in adverse events and a 7-fold increase in relapses as detailed in Teva's seventh Citizen Petition (FDA-2014-P-0933-0001 available at www.regulations.gov/#?documentDetail;D=FDA-2014-P-0933-0001, and also described herein).

The gene expression findings suggest that regardless of the many issues in the design and conduct of Synthon's GATE study that render the results unreliable, the study itself was premature because it was conducted on a glatiramoid that is almost certainly not equivalent to Copaxone®. Together, these data warrant further investigation, and emphasize the need for clinical trials following upon the establishment of quality and pharmaceutical equivalence, specifically multi-year safety studies with standard clinical endpoints for MS, to ensure the safety and well-being of multiple sclerosis patients.

How best to determine whether a differently manufactured glatiramoid is as safe and effective as Copaxone® remains an unresolved question that is also the subject of active research. One such differently manufactured glatiramoid, Synthon's Polimunol, purports to have been tested in a clinical study (GATE), yet the trial results are of questionable validity and raise more questions than answers, as detailed in Appendix 2 of FDA-2014-P-0933-0001 available at www.regulations.gov/#?documentDetail;D=FDA-2014-P-0933-0001, and also described herein]. Beyond the extensive flaws in the design and conduct of the GATE study that render the results unreliable, the study itself was premature because the equivalence of Synthon's glatiramoid with Copaxone® had not been conclusively established, and data from a full battery of high resolution physicochemical and gene expression studies support the conclusion that Sython's glatiramoid and Copaxone® are not equivalent.

The newest gene expression studies first reported in this document find extensive and significant differences between the gene expression profiles modulated by Synthon's Polimunol and the profiles modulated by Copaxone®, in both human monocytes and mouse splenocytes. Both of these studies employ a whole genome based approach, looking across the entire expression array in an unbiased fashion, in model systems capturing differing, relevant aspects of Copaxone® effect.

In mouse splenocytes, 223 probesets were significantly differentially expressed between Copaxone® and Polimunol in Copaxone®-immunized mice, and 431 probesets were significantly differentially expressed between Copaxone® and Polimunol in Polimunol-immunized mice. In both immunization conditions, IL18 was downregulated to a significantly greater extent by Copaxone® than Polimunol, raising concerns about Polimunol's reduced effectiveness in suppressing this MS-associated cytokine. As described above, IL18 is known to affect helper T cell differentiation, an important aspect of Copaxone's mechanism of action, and to induce IFNg expression, and has been implicated in MS pathogenesis. IL18 induces proinflammatory Th1 responses and has been implicated in a number of neurodegenerative and neuroinflammatory contexts including MS (Felderhoff-Mueser et al, Trends Neurosci, 2005).

The probesets differentially expressed between Copaxone® and Polimunol enriched to 22 pathways for Copaxone® immunization, and 10 pathways for Polimunol immunization. Many of these pathways overlapped, including immune response and response to virus. The fact that there are many significant differences between the gene expression patterns induced in splenocytes by Polimunol and Copaxone, regardless of whether those splenocytes are from mice immunized with Polimunol or with Copaxone®, further emphasizes the robustness of the observed differences between these two medicines. Moreover, Polimunol upregulates a number of interferon-related genes, including RIG-I, MDA5, and IRF7, suggesting the possibility of increased type I interferon signaling with Polimunol treatment. An increase in type I interferon is concerning, and could lead to possible adverse events such as flu-like symptoms.

In human monocytes, 779 probesets were differentially expressed, with 137 pathways enriched among the probesets upregulated by Polimunol relative to Copaxone®. Differentially expressed genes included CYP1B1 and IL1B, and the pathways enriched among the differentially expressed probesets included cytokine-cytokine receptor interactions and response to lipopolysaccharide. The enrichment in the response to lipopolysaccharide (LPS) pathway is particularly concerning, because the same prototypic pro-inflammatory pathway was also enriched among genes differentially expressed by the purported generic Probioglat, and the introduction of Probioglat in Mexico was associated with a 3-fold increase in adverse events (and a 7-fold increase in relapses) as described in Teva's seventh Citizen Petition (FDA-2014-P-0933-0001 available at www.regulations.gov/#?documentDetail;D=FDA-2014-P-0933-0001, and also described herein). Whether a similar phenomenon potentially underlies the higher adverse event rate (˜2.4-fold increase) observed in Synthon's GATE trial warrants further investigation.

Taken together, the new data introduced here demonstrates that the genes and pathways modulated by Synthon's Polimunol differ significantly and extensively from those modulated by Copaxone®. These data are in line with Teva's report of significant differences observed in various state-of-the-art physicochemical analyses detailed in a November comment to CP Docket FDA-2014-P-0933 (available at www.noticeandcomment.com/FDA-2014-P-0933-fcol-41234.aspx).

The biology underlying the differences detailed herein provide potential mechanistic hypotheses for differential efficacy and for the increase in adverse events leading to discontinuations as reported by Synthon with Polimunol relative to Copaxone® in the GATE study. Several striking biological differences stand out including levels of inflammatory cytokine genes such as IL18, interferon genes, and pro-inflammatory pathways such as response to lipopolysaccharide. These findings warrant further investigation, and raise substantial concerns for the safety and well-being of patients treated with Synthon's Polimunol. The data further illustrates the imminent need for guidelines specific to NBCD, and specifically to glatiramoids. The gene expression changes described in this document, together with physicochemical differences between Polimunol and Copaxone® described in a Nov. 13, 2014 comment to CP Docket FDA-2014-P-0933-0020 (available at www.noticeandcomment.com/FDA-2014-P-0933-fcol-41234.aspx), support the conclusion that Synthon's Polimunol is not therapeutically equivalent or similar to Copaxone®.

More generally, to establish whether a follow on glatiramoid has comparable safety and effectiveness to Copaxone®, it must first be established that the glatiramoid is the same as Copaxone® using a full battery of orthogonal methodologies including assays that detect high-resolution physicochemical properties and functionally relevant biological properties, including genome-wide gene expression analyses in immunologically relevant model systems including systems modeling antigen presenting cells and systems modeling T cells. Only when an applicant has demonstrated equivalence on these biological and physicochemical methods should they proceed to establish therapeutic comparability through an adequately powered, at least 2-year clinical trial using a widely accepted clinical endpoint such as annualized relapse rate.

### Example 12

### Gene Expression Studies of a Human Monocyte Cell Line Identify Dissimilarities Between Differently Manufactured Glatiramoids

Protein Levels at 24h are Consistent with Upregulation of Pro-Inflammatory mRNA Markers by Probioglat Versus GA Treatment at 6h

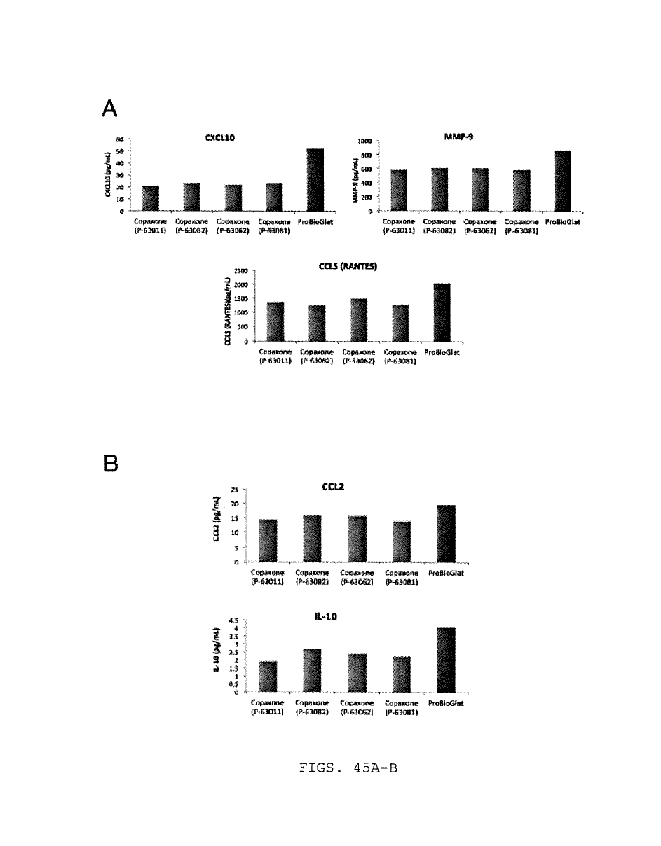

Protein concentration was tested in the same experiment at the 24h timepoint in order to validate upregulation of pro-inflammatory markers by Probioglat versus GA. Taking into consideration the fact that differences observed at the mRNA level do not necessarily translate to protein concentration differences, and may reflect regulatory processes, a Luminex kit measuring the concentrations of a panel of 45 chemokines and cytokines (in pg/ml) was employed. The Bio-Plex Human Chemokine (Bio Rad kit) and the Luminex Performance Assay (R&D kit) were utilized. Protein concentrations were measured in a single replicate at 24 hours, a timepoint estimated to correspond to the time when the mRNA signals observed at 6 hours may have been translated to protein. Of the five genes tested by qRT-PCR, three were represented on the Luminex panel: CCL5, CXCL10, and MMP9. All three showed higher concentrations in the Probioglat samples than the GA samples (fold changes 1.5, 2.3, and 1.4, respectively; FIG. 45) consistent with the directionality of the gene expression data (FIGS. 4A-D). Two other genes discussed above, IL10 and CCL2, were also present on the Luminex panel and also showed higher concentration levels in Probioglat relative to GA (fold changes 1.8 and 1.3, respectively; FIG. 45), consistent with the directionality observed at the mRNA level (FIG. 41d-e).

Key Genes Upregulated by Probioglat Compared to GA were Validated in Primary Human Monocytes

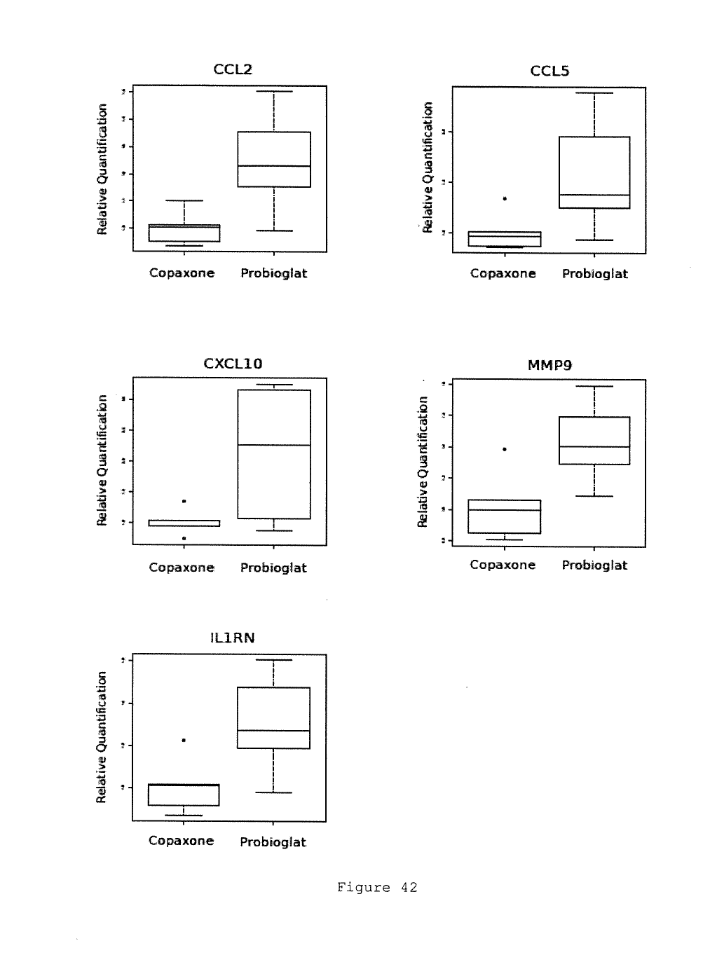

While immortalized cell lines are widely utilized in biological research and provide various advantages including uniformity and accessibility, it is important to confirm that the changes introduced by the immortalization process do not alter the key results. Therefore, top findings from the expression data were further tested in primary monocytes from healthy human donors using the sensitive method of qRT-PCR. Nine genes (CCL2, CCL5, CXCL10, MMP1, MMP9, CD9, ICAM1, IL10, IL1RN) were chosen for testing based on the findings reported above from the THP-1 monocyte cell line. In primary monocytes from a healthy donor with 6 replicates, the majority of the tested genes exhibited the expected directionality of expression differences between Probioglat and GA. Five of these nine genes passed statistical significance (FIG. 42). These genes included IL1RN, and the pro-inflammatory CCL2, CCL5, CXCL10 and MMP9 (p values<0.01 and 0.009, 0.029, 0.02, and 0.009, respectively).

### Discussion

### Example 12

Protein concentrations tested in the same experiment at the 24h timepoint were consistent with the findings observed at the mRNA level, supporting the reported findings and indicating an inflammation-related biological impact at the protein level. An independent follow-on study in primary human monocytes tested nine inflammation and MS-related genes by qRT-PCR, finding that five of these genes were statistically significantly upregulated by Probioglat relative to GA. These included IL1RN, which is relevant to Copaxone mechanism of action. In addition, CCL2, CCL5, CXCL10, and MMP9 were all seen to be upregulated significantly and consistently at both the mRNA and protein level in THP-1 cells, as well as confirmed by qRT-PCR in primary human monocytes. These genes act in pro-inflammatory pathways and have been implicated as relevant to MS susceptibility and severity, as described above.

The complex picture of genomic signatures described here underscores the intricate relationships between immune processes, effects of treatment on the associated pathways and the differing responses of each immune cell type. Consistent with previous evidence from other systems and cell types (Huang et al, Nucleic Acids Res. 2009), differences are consistently observed between GA and differently-manufactured glatiramoids, although their nature depends on the biological context of the tested model. Further, many of these differences affect molecules relevant to drug MOA and MS disease pathoetiology, particularly relating to inflammatory signatures. Genes significantly upregulated by Probioglat relative to GA were significantly enriched for inflammatory pathways and included key pro-inflammatory genes.

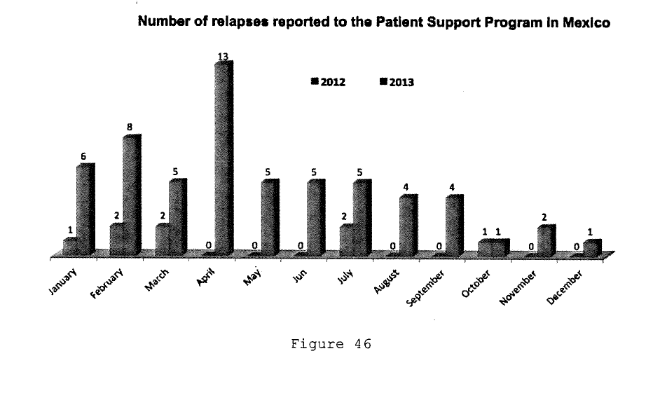

These findings have identified significant differences that warrant further investigation, especially in light of the observed clinical effects of Probioglat's introduction. The Teva Patient Support Program in Mexico records numbers of adverse events, including during the years 2012 and 2013 (Table 31; FIG. 46), for which a similar number of patients were tracked (1618 patients in 2012; 1552 in 2013). Probioglat was first introduced in January 2013; subsequently, each time a patient's prescription was filled, it could be with either Probioglat or GA. In 2013 versus 2012 (during which only branded GA was marketed), numbers of adverse events, and relapses specifically, increased more than 3-fold (from 125 to 380) and 7-fold (from 8 to 59), respectively (Table 31; FIG. 46), representing statistically-significant increases (p<2.2e-29 and p<3e-11, respectively). The gene-expression differences observed herein warrant careful investigation, through studies comparing GA to candidate generics in meaningful settings, most comprehensively including clinical trials.

TABLE 31
Reported Adverse Events and Relapses in Teva's Patient
Support Program, 2012-2013.

| | 2012 | 2013 | Statistical Significance |
|---|---|---|---|
| | (Copaxone | (Probioglat + | of Increase |
| | alone) | Copaxone) | from 2012 to 2013 |
| Adverse Events | 125 | 380 | p < 2.2e−29* |
| Adverse Event Rate | 10.4 | 31.7 | |
| (per month) | | | |
| Relapses | 8 | 59 | p < 3e−11* |
| Relapse Rate (per | 0.7 | 4.9 | |
| month) | | | |
| Total Number of | 1618 | 1552 | |
| Patients in Teva's | | | |
| Patient Support | | | |
| Program | | | |

*Single tailed p-value by Fisher's Exact test comparing the number of adverse events and relapses in 2012 < 2013.

### Example 13

### Validation of Key mRNA-Based Findings by Secreted Protein Analyses

To evaluate effects of Copaxone at the protein level, and assess the extent to which differences observed at the mRNA level (using micaroarry and qRT-PCR) were further consistent with effects observed at the level of the translated proteins, levels of 42 secreted cytokines and chemokines were measured using Luminex technology, analyzing the supernatant of THP-1 cells (human monocyte cell line) stimulated for 24 hours with Copaxone, FOGA, or mannitol control.

After applying statistical significance threshold, Synthon's Polimunol treatment increased the levels of 23 proteins (out of 39 having sufficient measurements to test for this comparison) relative to Copaxone, while Probioglat treatment increased the levels of 31 proteins (out of 40 having sufficient measurements to test for this comparison) relative to Copaxone (Table 32).

### Secreted Protein Analyses

Measurements were also undertaken at the protein level, in order to assess the biological relevance of the observed gene expression differences. Levels of a panel of secreted cytokines and chemokines were measured via Luminex from THP-1 cells stimulated with branded GA, Polimunol, or Probioglat.

Cells from the THP-1 human monocyte cell line were incubated with branded GA, FOGA (Polimunol or Probioglat), or vehicle control (mannitol) for 24 hours. This time point was chosen in order to reflect translation of mRNA expression patterns observed in the same study design at 6 hours post stimulation. Supernatants were collected and assayed for the concentration of selected proteins using a Luminex assay. A total of 5 biological replicates per condition were utilized.

Data (concentrations in pg/mL) were compared using a two-sided t-test with equal variance, and corrected for multiple hypothesis testing using Benjamini-Hochberg correction. To calculate the fold change between the protein expression levels with FOGA and with GA, GA values were averaged together and compared to the average value for FOGA (average FOGA expression level/average GA expression level).

### Polimunol Versus Copaxone

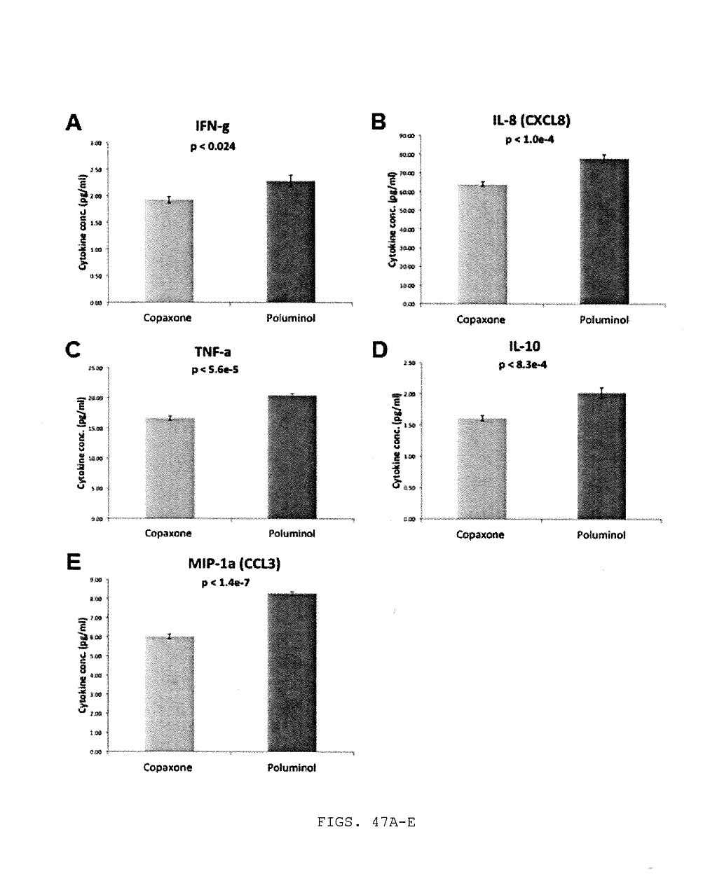

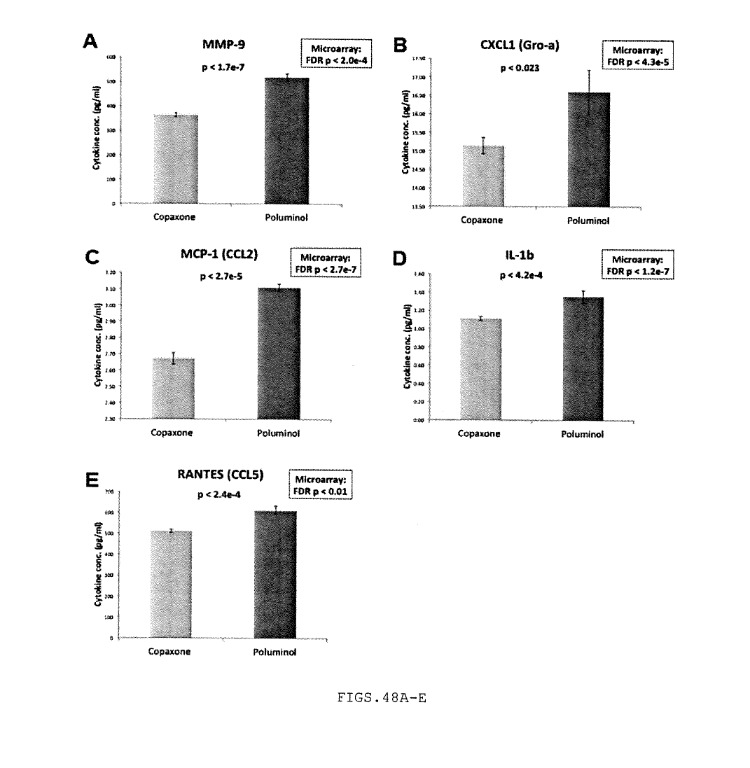

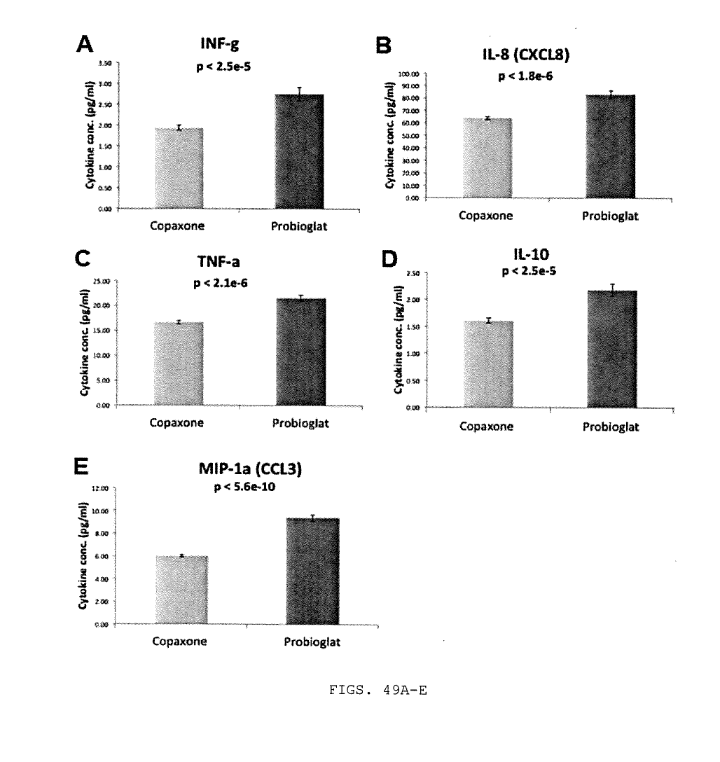

Polimunol treatment significantly increased the levels of 23 proteins (out of 39 having sufficient measurements to test for this comparison). These include IFNg, TNFa, MIP-1a (CCL3), IL-8 (CXCL8), and IL-10 (FIG. 47; adjusted p values as shown). Many of these proteins were also increased in level by Copaxone treatment relative to mannitol control. Consistent with the result observed at the mRNA expression level, secreted levels of MMP-9, MCP-1 (CCL2), RANTES (CCL5), Gro-a (CXCL1), and IL-1b were increased with Polimunol treatment relative to GA (FIG. 48; adjusted p values as shown). Of note, CCL2 was not modulated by GA relative to control, while it was increased by Polimunol treatment relative to GA.

A number of important differences in secreted protein level are observed for Polimunol relative to Copaxone, including levels of IFNg, TNFa, IL-8, and MIP-1a. IFNg is the major cytokine that drives the pro-inflammatory Th1 T-cell response, and is both implicated in MS and known to be relevant to Copaxone mechanism of action (Gilli et al 2012; Tumani et al 2011). TNFa is a major cytokine directing inflammatory responses, and polymorphisms in this gene have been reported to influence MS susceptibility (Rahmanian et al, 2014). The chemokine IL-8 (CXCL8) has been implicated in MS disease progression and risk, and proposed as a biomarker for MS (Lund et al, 2004; Kelland et al, 2011; Almasi et al, 2013; Tomioka et al, 2014). MIP-1a (Macrophage Inflammatory Protein 1-alpha; CCL3) is another proinflammatory chemokine with relevance to MS (Zhang et al, Brain, 2000; Aung et al, 2010).

Differences are also observed in the level of secreted IL-10. IL-10 is an anti-inflammatory cytokine important for Th2 polarization, and has been implicated in the mechanism of action of Copaxone (Vieira at al. Glatiramer acetate (copolymer-1, copaxone) promotes Th2 cell development and increased IL-10 production through modulation of dendritic cells. J. Immunol. 1950 170, 4483-4488 (2003); Kim, H. J. et al. Type 2 monocyte and microglia differentiation mediated by glatiramer acetate therapy in patients with multiple sclerosis. J. Immunol. 1950 172, 7144-7153 (2004); Weber, M. S. et al. Type II monocytes modulate T cell-mediated central nervous system autoimmune disease. Nat. Med. 13, 935-943 (2007)).

The observed differences in levels of each of these five proteins with Polimunol versus Copaxone treatment confirm the gene level reports by microarray as described (Kolitz et al, Sci Rep, 2015, in press). As detailed above, CCL2 and RANTES (CCL5) are inflammatory chemoattractants with known relevance to MS (Allie et al 2005; Mahad et al 2006), and MMP-9 is a pro-inflammatory matrix metalloproteinase extensively implicated in MS (Rosenberg 2005; Romme Christensen et al 2013; Kouwenhoven et al 2002; Waubant et al 2006; Milward et al 2008). The biological relevance of these genes is discussed at length in Section 6.2. Gro-a (CXCL1) is another chemokine implicated in MS and MS models (Glabinski et al 1998; Rumble et al, J Exp Med, 2015), and IL-1b is a proinflammatory cytokine important for inflammasome signalling, with numerous connections to MS in the literature (Prins et al 2013; Rossi et al 2014; Murta et al 2015).

Overall the proteomic data complements the microarray data and confirms the robustness of the detected signatures. These observations substantiate the concerns raised by upregulation of proinflammatory cytokines and associated pathways.

### Probioglat Versus Copaxone

Key differences between Probioglat and Copaxone were identified using microarray in THP-1 cells, then tested in primary monocytes, confirming the relevance of the cell line model. To see whether these gene expression differences translated into differences at the protein level, indicating biological relevance, secreted protein levels were measured from THP-1 cells treated with Copaxone or Probioglat.

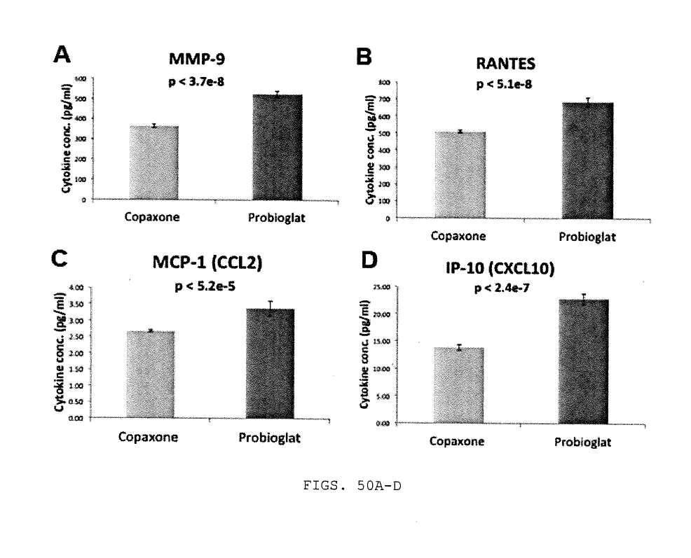

Probioglat treatment significantly increased the levels of 31 proteins (out of 40 having sufficient measurements to test for this comparison). These include IFNg, TNFa, MIP-1a (CCL3), IL-8 (CXCL8), and IL-10 (FIG. 49; adjusted p values as shown). The clear biological relevance of these proteins to disease or to GA response is also described above. Many of these proteins were also increased in level by GA treatment relative to mannitol control. Consistent with results observed at the mRNA expression level (Kolitz et al, Sci Rep 2015, in press), the 31 proteins also include MMP-9, IP-10 (CXCL10), MCP-1 (CCL2) and RANTES (CCL5) (FIG. 50; adjusted p values as shown). Of note, CCL2 was not modulated by GA relative to control, while it was increased by Probioglat treatment relative to GA.

Secreted levels of several key proteins were observed to differ with Probioglat treatment relative to Copaxone, including IFNg, TNFa, IL-8, and MIP-1a. As discussed in the previous section, each of these genes has important roles in inflammation and/or MS (Gilli et al 2012; Tumani et al 2011; Rahmanian et al, 2014; Lund et al, 2004; Kelland et al, 2011; Almasi et al, 2013; Tomioka et al, 2014; Zhang et al, Brain, 2000; Aung et al, 2010). The level of secreted IL-10 also differs between Probioglat and Copaxone treatment. As described in the previous section, IL-10 is an anti-inflammatory cytokine important for Th2 polarization, and implicated in Copaxone mechanism of action (Vieira at al. 2003; Kim, H. J. et al. 2004; Weber, M. S. et al. 2007).

As described above, CCL2, RANTES (CCL5) and IP-10 (CXCL10) are all inflammatory chemoattractants with relevance to MS (Allie et al 2005; Mahad et al 2006; Peperzak et al 2013; Thamilarasan et al 2013), and MMP-9 is a pro-inflammatory matrix metalloproteinase implicated in MS (Rosenberg 2005; Romme Christensen et al 2013; Kouwenhoven et al 2002; Waubant et al 2006; Milward et al 2008). As described in FDA-2014-P-0933; Kolitz et al, Sci Rep, 2015, in press; and above, the genes coding for these proteins were also demonstrated to be induced to differing degrees by Probioglat and Copaxone in both the THP-1 model system and in primary human monocytes. The biological relevance of these genes is discussed at length above.

Thus, the protein analyses further supported the observation of inflammation-related differences between Probioglat and Copaxone, consistent with the clinical observations of increased rates of adverse events and relapses.

TABLE 32
Proteins and genes significantly differentially expressed
between Polimunol and Copaxone ® treatment and between
Probioglat and Copaxone ® treatment in THP-1 human monocytes.

| | | Polimunol vs Copaxone | | | Probioglat vs Copaxone | | |
|---|---|---|---|---|---|---|---|
| | Gene | | | Benjamini | | | Benjamini |
| Protein | symbol | FC | p. value | p value | FC | p. value | p value |
| MIP-1a | CCL3 | 1.37 | 3.59E−09 | 1.40E−07 | 1.56 | 1.58E−11 | 5.62E−10 |
| MMP-9 | MMP9 | 1.42 | 8.69E−09 | 1.69E−07 | 1.44 | 7.40E−09 | 3.70E−08 |
| MDC | CCL22 | 1.13 | 1.63E−07 | 2.12E−06 | 1.24 | 2.81E−11 | 5.62E−10 |
| CCL24 | CCL24 | 1.15 | 2.39E−07 | 2.33E−06 | 1.25 | 2.31E−10 | 2.84E−09 |
| CX3CL1 | CX3CL1 | 1.28 | 2.28E−06 | 1.78E−05 | 1.64 | 3.15E−09 | 1.80E−08 |
| MIP-3a | CCL20 | 1.28 | 4.73E−06 | 2.65E−05 | 1.72 | 2.84E−10 | 2.84E−09 |
| MCP-1 | CCL2 | 1.16 | 4.75E−06 | 2.65E−05 | 1.26 | 2.48E−05 | 5.23E−05 |
| TNF-a | TNF | 1.23 | 1.16E−05 | 5.64E−05 | 1.29 | 7.81E−07 | 2.08E−06 |
| IL-8 | IL8 | 1.21 | 2.40E−05 | 1.04E−04 | 1.30 | 6.39E−07 | 1.83E−06 |
| MCP-4 | CCL13 | 1.21 | 4.98E−05 | 1.94E−04 | 1.44 | 2.66E−09 | 1.77E−08 |
| RANTES | CCL5 | 1.19 | 6.70E−05 | 2.38E−04 | 1.34 | 1.15E−08 | 5.13E−08 |
| IL-1b | IL1B | 1.22 | 1.30E−04 | 4.22E−04 | 1.43 | 2.53E−07 | 8.44E−07 |
| MCP-2 | CCL8 | 1.19 | 1.76E−04 | 5.27E−04 | 1.50 | 7.15E−10 | 5.72E−09 |
| IL-10 | IL10 | 1.25 | 2.99E−04 | 8.34E−04 | 1.36 | 1.07E−05 | 2.53E−05 |
| I-TAC | CXCL11 | 1.21 | 8.60E−04 | 2.24E−03 | 1.40 | 3.03E−05 | 6.06E−05 |
| CXCL13 | CXCL13 | 1.31 | 1.35E−03 | 3.25E−03 | 1.68 | 9.06E−08 | 3.30E−07 |
| IP-10 | CXCL10 | 1.28 | 1.42E−03 | 3.25E−03 | 1.64 | 5.88E−08 | 2.35E−07 |
| MCP-3 | CCL7 | 1.43 | 5.11E−03 | 1.11E−02 | 1.57 | 1.38E−03 | 2.21E−03 |
| CCL1 | CCL1 | 1.20 | 5.73E−03 | 1.18E−02 | 1.26 | 1.57E−03 | 2.35E−03 |
| CXCL1 | CXCL1 | 1.10 | 1.19E−02 | 2.32E−02 | 1.22 | 1.32E−05 | 2.93E−05 |
| INF-g | IFNg | 1.18 | 1.32E−02 | 2.45E−02 | 1.42 | 1.05E−05 | 2.53E−05 |
| CCL26 | CCL26 | 1.16 | 1.91E−02 | 3.39E−02 | 1.32 | 1.59E−03 | 2.35E−03 |
| MIF | MIF | 1.23 | 2.86E−02 | 4.85E−02 | 1.38 | 5.54E−04 | 1.01E−03 |
| IL-16 | IL16 | 1.12 | 3.15E−02 | 5.13E−02 | 1.25 | 9.01E−05 | 1.72E−04 |
| IL-6 | IL6 | 1.19 | 3.37E−02 | 5.26E−02 | 1.33 | 1.06E−03 | 1.84E−03 |
| CXCL6 | CXCL6 | 1.18 | 4.02E−02 | 6.03E−02 | 1.16 | 6.56E−02 | 7.96E−02 |
| TECK | CCL25 | 1.11 | 5.67E−02 | 8.19E−02 | 1.44 | 3.77E−07 | 1.16E−06 |
| GM-CSF | CSF2 | 1.22 | 9.66E−02 | 1.35E−01 | 1.30 | 4.65E−02 | 5.81E−02 |
| IL-2 | IL2 | 1.16 | 1.11E−01 | 1.49E−01 | 1.34 | 1.20E−03 | 2.00E−03 |
| MPIF-1 | CCL23 | 1.05 | 2.48E−01 | 3.22E−01 | 1.02 | 6.91E−01 | 7.47E−01 |
| MIP-3b | CCL19 | 1.11 | 2.72E−01 | 3.42E−01 | 1.25 | 1.93E−02 | 2.57E−02 |
| SDF-1a + b | CXCL12 | 1.05 | 3.41E−01 | 4.15E−01 | 1.07 | 2.55E−01 | 2.92E−01 |
| CXCL2 | CXCL2 | 1.17 | 4.06E−01 | 4.80E−01 | 1.65 | 2.85E−03 | 4.07E−03 |
| MIG | CXCL9 | 1.07 | 4.43E−01 | 5.08E−01 | 1.31 | 5.46E−03 | 7.54E−03 |
| CCL11 | CCL11 | 0.99 | 4.61E−01 | 5.13E−01 | 1.00 | 8.61E−01 | 9.06E−01 |
| CXCL5 | CXCL5 | 0.93 | 5.16E−01 | 5.59E−01 | 0.79 | 3.05E−02 | 3.94E−02 |
| MIP-1d (M [symbol] | CCL15 | 0.97 | 5.45E−01 | 5.74E−01 | 1.00 | 9.22E−01 | 9.22E−01 |
| SCYB16 | CXCL16 | 1.01 | 6.77E−01 | 6.94E−01 | 1.02 | 5.44E−01 | 6.05E−01 |
| CCL21 | CCL21 | 1.01 | 8.27E−01 | 8.27E−01 | 1.00 | 8.96E−01 | 9.19E−01 |
| IL-4 | IL4 | NA | NA | NA | 1.26 | 1.62E−01 | 1.91E−01 |
| TARC | CCL17 | 0.76 | NA | NA | 1.39 | NA | NA |
| CCL27 | CCL27 | NA | NA | NA | NA | NA | NA |

[symbol]

indicates data missing or illegible when filed

### REFERENCES

1. Noseworthy J H, Lucchinetti C, Rodriguez M, Weinshenker B G. Multiple sclerosis. N Engl J Med 2000; 343:938-52.
2. Guideline on clinical investigation of medicinal products for the treatment of multiple sclerosis EMEA, London 16 Sep. 2006.
3. Bjartmar C, Fox R J. Pathological mechanisms and disease progression of multiple sclerosis: therapeutic implications. Drugs of Today 2002; 38:17-29.
4. Fleming J O. Diagnosis and management of multiple sclerosis. 1st ed. New York: Professional communications, Inc., 2002.
5. Anderson D W, Ellenberg J H, Leventhal C M et al. Revised estimate of the prevalence of multiple sclerosis in the United States. Ann Neurol 1992; 31:333-36.
6. Compston A, Lassmann H, McDonald I. The story of multiple sclerosis. In: Compston A, Confavreux C, Lassman H, Mcdonald I, Miller D, Noseworthy J H, Smith K, Wekerle H, editors. McAlpine's Multiple Sclerosis. London: Churchill Livingstone; 2006. p. 3-68.
7. Revel M., Pharmacol. Ther., 100(1):49-62 (2003).
8. Copaxone© (glatiramer acetate injection) (2012) Teva Pharmaceutical Industries, Ltd Petach-Tikva, Israel:Rev-2/209.
9. Varkony H, et al. (2009) The glatiramoid class of immunomodulator drugs. Expert Opin Pharmacother 10:657-668.
10. Filippi M, et al. (2001) Glatiramer acetate reduces the proportion of new M S lesions evolving into “black holes”. Neurology 57:731-733.
11. Kipnis J, Schwartz M (2002) Dual action of glatiramer acetate (Cop-1) in the treatment of CNS autoimmune and neurodegenerative disorders. TRENDS in Molecular Medicine 8:319-323.
12. Teitelbaum D, Aharoni R, Arnon R, Sela M (1988) Specific inhibition of the T-cell response to myelin basic protein by the synthetic copolymer Cop 1. Proc Natl Acad Sci USA 85:9724-9728.
13. Putheti P, Soderstrom M, Link H, Huang Y M (2003) Effect of glatiramer acetate (Copaxone®) on CD4+CD25high T regulatory cells and their IL-10 production in multiple sclerosis. J Neuroimmunol 144:125-131.
14. Stadelmann C, et al. (2002) BDNF and gp145trkB in multiple sclerosis brain lesions: neuroprotective interactions between immune and neuronal cells? Brain 125:75-85.
15. Ford C, et al. (2010) Continuous long-term immunomodulatory therapy in relapsing multiple sclerosis: results from the 15-year analysis of the US prospective open-label study of glatiramer acetate. Mutt Scler 16:342-350.
16. Aharoni R, Teitelbaum D, Arnon R, Sela M (1999) Copolymer 1 acts against the immunodominant epitope 82-100 of myelin basic protein by T cell receptor antagonism in addition to major histocompatibility complex blocking. Proc Natl Acad Sci USA 96:634-639.
17. Arnon R, Aharoni R (2004) Mechanism of action of glatiramer acetate in multiple sclerosis and its potential for the development of new applications. Proc Natl Acad Sci USA 101 Suppl 2:14593-14598.
18. Teitelbaum D, Aharoni R, Sela M, Arnon R (1991) Cross-reactions and specificities of monoclonal antibodies against myelin basic protein and against the synthetic copolymer 1. Proc Natl Acad Sci USA 88:9528-9532.
19. Webb C, Teitelbaum D, Arnon R, Sela M (1973) In vivo and in vitro immunological cross-reactions between basic encephalitogen and synthetic basic polypeptides capable of suppressing experimental allergic encephalomyelitis. Eur J Immunol 3:279-286.
20. Duda P W, et al. (2000) Human and murine CD4 T cell reactivity to a complex antigen: recognition of the synthetic random polypeptide glatiramer acetate. J Immunol 165:7300-7307.
21. Duda P W, et al. (2000) Glatiramer acetate (Copaxone) induces degenerate, Th2-polarized immune responses in patients with multiple sclerosis. J Clin Invest 105:967-976.
22. Brenner T, et al. (2001) Humoral and cellular, immune responses to Copolymer 1 in multiple sclerosis patients treated with Copaxone. J Neuroimmunol 115:152-160.
23. Aharoni R, Teitelbaum D, Sela M, Arnon R (1997) Copolymer 1 induces T cells of the T helper type 2 that crossreact with myelin basic protein and suppress experimental autoimmune encephalomyelitis. Proc Natl Acad Sci USA 94:10821-10826.
24. Wiesemann E, et al. (2003) Correlation of serum IL-13 and IL-5 levels with clinical response to Glatiramer acetate in patients with multiple sclerosis. Clin Exp Immunol 133:454-460.
25. Aharoni R, Teitelbaum D, Sela M, Arnon R (1998) Bystander suppression of experimental autoimmune encephalomyelitis by T cell lines and clones of the Th2 type induced by copolymer 1. J Neuroimmunol 91:135-146.
26. Aharoni R, et al. (2003) Glatiramer acetate-specific T cells in the brain express T helper 2/3 cytokines and brain-derived neurotrophic factor in situ. Proc Natl Acad Sci USA 100:14157-14162.
27. Miller A, et al. (1998) Treatment of multiple sclerosis with copolymer-1 (Copaxone): implicating mechanisms of Th1 to Th2/Th3 immune-deviation. J Neuroimmunol 92:113-121.
28. Neuhaus 0, et al. (2000) Multiple Sclerosis: Comparison of copolymer-1-reactive T cell Lines from treated and untreated subjects reveals cytokine shift From T helper 1 to T helper 2 cells. Proceedings of the National Academy of Sciences 97:7452-7457.
29. Venken K, et al. (2008) Natural naive CD4+CD25+CD127low regulatory T cell (Treg) development and function are disturbed in multiple sclerosis patients: recovery of memory Treg homeostasis during disease progression. J Immunol 180:6411-6420.
30. Haas J, et al. (2009) Glatiramer acetate improves regulatory T-cell function by expansion of naive CD4(+)CD25(+)FOXP3(+)CD31(+) T-cells in patients with multiple sclerosis. J Neuroimmunol 216:113-117.
31. Hong J, et al. (2005) Induction of CD4+CD25+ regulatory T cells by copolymer-I through activation of transcription factor Foxp3. Proc Natl Acad Sci USA 102:6449-6454.
32. Weber M S, et al. (2007) Type II monocytes modulate T cell-mediated central nervous system autoimmune disease. Nat Med 13:935-943.
33. Xu D, et al. (1998) Selective expression and functions of interleukin 18 receptor on T helper (Th) type 1 but not Th2 cells. J Exp Med 188:1485-1492.
34. Jee Y, et al. (2007) CD4(+)CD25(+) regulatory T cells contribute to the therapeutic effects of glatiramer acetate in experimental autoimmune encephalomyelitis. Clin Immunol 125:34-42.
35. Windhagen A, et al. (1998) Cytokine secretion of myelin basic protein reactive T cells in patients with multiple sclerosis. J Neuroimmunol 91:1-9.
36. Neuhaus 0, et al. (2000) Multiple sclerosis: comparison of copolymer-1-reactive T cell lines from treated and untreated subjects reveals cytokine shift from T helper 1 to T helper 2 cells. Proc Natl Acad Sci USA 97:7452-7457.
37. Chen M, et al. (2001) Glatiramer acetate induces a Th2-biased response and crossreactivity with myelin basic protein in patients with MS. Mutt Scler 7:209-219.
38. Franciotta D, et al. (2003) Interferon gamma and interleukin 4 producing T cells in peripheral blood of multiple sclerosis patients undergoing immunomodulatory treatment. J Neurol Neurosurg Psychiatry 74:123-126.
39. Weder C, et al. (2005) Clinical and immune responses correlate in glatiramer acetate therapy of multiple sclerosis. Eur J Neurol 12:869-878.
40. Sanna A, et al. (2006) Glatiramer acetate reduces lymphocyte proliferation and enhances IL-5 and IL-13 production through modulation of monocyte-derived dendritic cells in multiple sclerosis. Clin Exp Immunol 143:357-362.
41. Kipnis J, Avidan H, Caspi R R, Schwartz M (2004) Dual effect of CD4+CD25+ regulatory T cells in neurodegeneration: a dialogue with microglia. Proc Natl Acad Sci USA 101 Suppl 2:14663-14669.
42. Tennakoon D K, et al. (2006) Therapeutic induction of regulatory, cytotoxic CD8+ T cells in multiple sclerosis. J Immunol 176:7119-7129.
43. Praksova P, et al. (2012) Immunoregulatory T cells in multiple sclerosis and the effect of interferon beta and glatiramer acetate treatment on T cell subpopulations. J Neurol Sci 319:18-23.
44. Achiron A, et al. (2009) Molecular profiling of glatiramer acetate early treatment effects in multiple sclerosis. Dis Markers. 27(2):63-73.
45. Dunning M J, Smith M L, Ritchie M E, Tavare S (2007) beadarray: R classes and methods for Illumina bead-based data. Bioinformatics 23:2183-2184.
46. Sharan R, Maron-Katz A, Shamir R (2003) CLICK and EXPANDER: a system for clustering and visualizing gene expression data. Bioinformatics 19:1787-1799.
47. Sugimoto N (2006) Foxp3-dependent and -independent molecules specific for CD25+CD4+ natural regulatory T cells revealed by DNA microarray analysis. International Immunology 18:1197-1209.
48. Subramanian N, et al. (2005) Gene set enrichment analysis: A knowledge-based approach for interpreting genome-wide expression profiles. Proc Natl. Acad Sci USA 102:15545-15550.
49. Weber M. S. et al. (2007) Type II monocytes modulate T cell-mediated central nervous system autoimmune disease. Nature Medicine 13:935-943.
50. Wang Z. (2006) Role of IFN-g in induction of Foxp3 and conversion of CD4+CD25-f cells to CD4+ Tregs. Journal of Clinical Investigation. doi:10.1172/JCl25826.
51. Hong J. et al. (2005) Induction of CD4+CD25+ regulatory T cells by copolymer-I through activation of transcription factor Foxp3. Proc Natl Acad Sci USA 102:6449-6454.
52. Burger D. et al. (2009) Glatiramer acetate increases IL-1 receptor antagonist but decreases T cell-induced I L-lp in human monocytes and multiple sclerosis. Proceedings of the National Academy of Sciences 106:4355-4359.
53. Ingersoll M. A. et al. (2010) Comparison of gene expression profiles between human and mouse monocyte subsets. Blood 115, e10-e19.
54. Nylander A. and Hafler D. A. (2012) Multiple sclerosis. Journal of Clinical Investigation 122:1180-1188.
55. Dhib-Jalbut S. et al. (2012) in ECTRIMS.
56. Bar-Or A. et al. (2003) Analyses of all matrix metalloproteinase members in leukocytes emphasize monocytes as major inflammatory mediators in multiple sclerosis. Brain 126:2738-2749.
57. Pul R. et al. (2012) Glatiramer Acetate Increases Phagocytic Activity of Human Monocytes In Vitro and in Multiple Sclerosis Patients. PLoS ONE 7, e51867.
58. Anis S. et al. (2013) in AAN.
59. Anis S. et al. (2010) in ECTRIMS.
60. Bolstad B. M., preprocessCore: A collection of pre-processing functions.
61. Johnson W. E. et al. (2007) Adjusting batch effects in microarray expression data using empirical Bayes methods. BiostatS 118-127.
62. Leek J. T. et al., sva: Surrogate Variable Analysis.
63. Ho J. W. K. et al. (2008) Differential variability analysis of gene expression and its application to human diseases. Bioinformatics 24:i390-398.
64. Quackenbush J. (2002) Microarray data normalization and transformation. Nature Genetics 32:496-501.
65. Pavlidis P. (2003) Using ANOVA for gene selection from microarray studies of the nervous system. Methods 31:282-289.
66. Benjamini Y. and Hochberg Y. (1995) Controlling the false discovery rate: a practical and powerful approach to multiple testing. Journal of the Royal Statistical Society. Series B (Methodological) 289-300.
67. Smyth G. K. (2004) Linear models and empirical bayes methods for assessing differential expression in microarray experiments. StatAppl Genet Mol Biol 3, Article3.
68. Smyth G. K. (2005) In Bioinformatics and Computational Biology Solutions Using R and Bioconductor, R. Gentleman, V. Carey, S. Dudoit, R. Irizarry, W. Huber, Eds. (Springer, New York) 397-420.
69. Gentleman R. C. et al. (2004) Bioconductor: Open software development for computational biology and bioinformatics. Genome Biology 5, R80.
70. Reich M. et al. (2006) GenePattern 2.0, Nature Genetics 38:500-501.
71. Saeys Y. et al. (2007) A review of feature selection techniques in bioinformatics. Bioinformatics 23:2507-2517.
72. Zheng Y. et al. (2007) Genome-wide analysis of Foxp3 target genes in developing and mature regulatory T cells. Nature 445:936-940.
73. Eppig J. T. et al. (2012) The Mouse Genome Database (MGD):
comprehensive resource for genetics and genomics of the laboratory mouse. Nucleic Acids Res 40:D881-D886.
74. Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat. Genet. 25, 25-29.
75. Benita Y. et al. (2010) Gene enrichment profiles reveal T-cell development, differentiation, and lineage-specific transcription factors including ZBTB25 as a novel NF-AT repressor. Blood 115:5376-5384.
76. Painter M. W. et al. (2011) Transcriptomes of the B and T lineages compared by multi-platform microarray profiling. Immunol 186:3047-3057.
77. Bakshi, S., Chalifa-Caspi, V., Plaschkes, I., Perevozkin, I.,
Gurevich, M., and Schwartz, R. (2013). Gene expression analysis reveals functional pathways of glatiramer acetate activation. Expert Opinion on Therapeutic Targets 17, 351-362.
78. Felderhoff-Mueser, U., Schmidt, 0.1., Oberholzer, A., Bührer, C., and Stahel, P. F. (2005). IL-18: a key player in neuroinflammation and neurodegeneration? Trends in Neurosciences 28, 487-493.
79. Huang, D. W., Sherman, B. T., and Lempicki, R. A. (2008). Systematic and integrative analysis of large gene lists using DAVID bioinformatics resources. Nature Protocols 4, 44-57.
80. Johnson, W. E., Li, C., and Rabinovic, A. (2007). Adjusting batch effects in microarray expression data using empirical Bayes methods. Biostatistics 8, 118-127.
81. Kanehisa, M., and Goto, S. (2000). KEGG: kyoto encyclopedia of genes and genomes. Nucleic Acids Res. 28, 27-30.
82. Karakas Celik, S., Oz, Z. S., Dursun, A., Unal, A., Emre, U., Cicek, S., and Keni, F. M. (2014). Interleukin 18 gene polymorphism is a risk factor for multiple sclerosis. Molecular Biology Reports 41, 1653-1658.
83. Leek J T, Johnson W E, Parker H S, Jaffe A E, Storey J D (2013). sva: Surrogate Variable Analysis.
84. Losy, J., and Niezgoda, A. (2001). IL-18 in patients with multiple sclerosis. Acta Neurologica Scandinavica 104, 171-173.
85. Nicoletti, F., Di Marco, R., Mangano, K., Patti, F., Reggio, E., Nicoletti, A., Bendtzen, K., and Reggio, A. (2001). Increased serum levels of interleukin-18 in patients with multiple sclerosis. Neurology 57, 342-344.
86. Orr, M., and Liu, P. (2009). Sample Size Estimation while Controlling False Discovery Rate for Microarray Experiments Using the ssize.fdr Package. The R Journal 1/1.
87. Shi, F.-D., Takeda, K., Akira, S., Sarvetnick, N., and Ljunggren, H.-G. (2000). IL-18 Directs Autoreactive T Cells and Promotes Autodestruction in the Central Nervous System Via Induction of IFN- by NK Cells. The Journal of Immunology 165, 3099-3104.
88. Smyth, G. K. (2004). Linear models and empirical bayes methods for assessing differential expression in microarray experiments. Stat Appl Genet Mol Biol 3, Article3.
89. Thompson, S. R., and Humphries, S. E. (2007). Interleukin-18 genetics and inflammatory disease susceptibility. Genes and Immunity 8, 91-99.
90. Towfic, F., Funt, J. M., Fowler, K. D., Bakshi, S., Blaugrund, E., Artyomov, M. N., Hayden, M. R., Ladkani, D., Schwartz, R., and Zeskind, B. (2014). Comparing the Biological Impact of Glatiramer Acetate with the Biological Impact of a Generic. PLoS ONE 9, e83757.
91. Trinchieri, G. (2010). Type I interferon: friend or foe? Journal of Experimental Medicine 207, 2053-2063.

1-100. (canceled)

101. In a process for producing a drug product comprising a glatiramer acetate related drug substance which involves an array of testing, the improvement comprising including in the array of testing the steps of:
a) obtaining a batch of the glatiramer acetate related drug substance or drug product;
b) contacting mammalian cells with an amount of the glatiramer acetate related drug substance or drug product obtained in step a);
c) determining the level expression of ABI2 by the mammalian cells; and
d) including the batch of the glatiramer acetate related drug substance in the production of the drug product if the level of expression of is not substantially identical to the level of expression of ABI2 by the same type of cells in the absence of the glatiramer acetate related drug substance under the same conditions.

102-149. (canceled)

150. The process of claim 101, wherein
contacting the mammalian cells in step (b) comprises i) administering to a mammal a predetermined amount of glatiramer acetate related drug substance or drug product of step (a), or ii) incubating the cells with an amount of the glatiramer acetate related drug substance or drug product of step (a), or a combination thereof; or
contacting the mammalian cells in step (b) comprises i) administering to a mammal a predetermined amount of glatiramer acetate related drug substance or drug product of step (a), or ii) incubating the cells with an amount of the glatiramer acetate related drug substance or drug product of step (a), or a combination thereof, wherein the mammal is human and the cells are peripheral mononuclear blood cells, the predetermined time point is 0, 1, 2, or 3 months or the incubation is for about 24 hours, for about 12 hours, or for about 6 hours.

151. The process of claim 101, wherein
contacting the mammalian cells in step (b) comprises i) administering to a mammal a predetermined amount of glatiramer acetate related drug substance or drug product of step (a), and ii) obtaining cells from the mammal at one or more predetermined time points; or
contacting the mammalian cells in step (b) comprises i) administering to a mammal a predetermined amount of glatiramer acetate related drug substance or drug product of step (a), and ii) obtaining cells from the mammal at one or more predetermined time points, wherein the mammal is human and the cells are peripheral mononuclear blood cells, the predetermined time point is 0, 1, 2, or 3 months or the incubation is for about 24 hours, for about 12 hours, or for about 6 hours.

152. The process of claim 101, wherein
contacting the mammalian cells in step (b) comprises incubating monocytic cell line cells with an amount of the glatiramer acetate related drug substance or drug product of step (a), thereby characterizing the glatiramer acetate related drug substance or drug product of step (a); or
contacting the mammalian cells in step (b) comprises incubating monocytic cell line cells with an amount of the glatiramer acetate related drug substance or drug product of step (a), thereby characterizing the glatiramer acetate related drug substance or drug product of step a) wherein the mammalian cells are THE-1 cells or the incubation is for about 24 hours, for about 12 hours, or for about 6 hours.

153. The process of claim 101, wherein
contacting the mammalian cells in step (b) comprises i) immunizing a mammal with a predetermined amount of glatiramer acetate related drug substance or drug product, ii) preparing a culture of cells from the mammal of step i) at one or more predetermined time points after immunization, and iii) incubating cells from the culture of cells obtained from the mammal with an amount of the glatiramer acetate related drug substance or drug product of step (a), thereby characterizing the glatiramer acetate related drug substance or drug product of step (a); or
contacting the mammalian cells in step (b) comprises i) immunizing a mammal with a predetermined amount of glatiramer acetate related drug substance or drug product, ii) preparing a culture of cells from the mammal of step i) at one or more predetermined time points after immunization, and iii) incubating cells from the culture of cells obtained from the mammal with an amount of the glatiramer acetate related drug substance or drug product of step (a), thereby characterizing the glatiramer acetate related drug substance or drug product of step (a), wherein
the glatiramer acetate related drug substance or drug product of step (iii) is the same glatiramer acetate related drug substance or drug product of step (i),
the glatiramer acetate related drug substance or drug product of step (iii) is a different glatiramer acetate related drug substance or drug product of step (i),
the incubation is for about 24 hours, for about 12 hours, or for about 6 hours or
the predetermined time point after immunization is 3 days.

154. The process of claim 101,
wherein the contacting of step (b) is in a cell culture;
wherein the contacting of step (b) is in a cell culture and the culture is a primary culture;
wherein the contacting of step (b) is in a cell culture and the culture is a primary culture, wherein the primary culture is a culture of spleen cells, wherein the primary culture is a culture of lymph node cells, or wherein the primary culture of spleen cells is prepared about 3 days after immunization;
wherein the contacting of step (b) is in a mammal;
wherein the contacting of step (b) is in a mammal and the mammal is a rodent or human; or
wherein the mammal of step (b) is a rodent or human;
wherein the mammal of step (b) is a mouse;
wherein the mammal of step (b) is a mouse, wherein the mouse is a female (SJL X BALB/C) F1 mouse, and wherein the mouse is about 8 to 12 weeks old;
wherein the cells of step (b) are of a type i) selected from the group of cell types consisting of FoxP3+ T cells, regulatory T cells, natural killer T cells, T helper 2 cells, CD8+ T cells, CD4+ T cells, B cells, macrophage cells, monocyte cells, eosinophils, dendritic cells, granulocytes, megakaryocytes, and myeloid progenitors; ii) selected from the group of cell types identified in Table 9; iii) selected from the group of cell types identified in Table 10; or iv) selected from the group of cell types identified in Table 11.

155. The process of claim 101,
wherein the level of expression is determined in hematological cells;
wherein the level of expression is determined in splenocytes;
wherein the level of expression is determined in monocytes;
wherein the level of expression is determined in monocytes, and wherein the monocytes are THP-1;
wherein the level of expression is determined in peripheral blood mononuclear cells; or
wherein the level of expression is determined in peripheral blond mononuclear cells and wherein the peripheral blood mononuclear cells are from a human.

156. The process of claim 101,
wherein the human has previously been treated with a glatiramer acetate related drug substance or drug product;
wherein the human is a naïve;
wherein the human is a glatiramoid naïve human; or
wherein the human is afflicted with RRMS.

157. The process of claim 101,
wherein the glatiramer acetate related drug substance or drug product is other than glatiramer acetate drug substance or drug product;
wherein the glatiramer acetate related drug substance is a glatiramoid or wherein the glatiramer acetate related drug product comprises a glatiramoid;
wherein the glatiramer acetate related drug substance is a glatiramoid other than glatiramer acetate drug substance; or
wherein the glatiramer acetate related drug product comprises a glatiramoid other than glatiramer acetate drug substance.

158. In the process of claim 101 comprising step (d), if the level of expression of ABI2 is upregulated relative to the level of expression by the same type of cells in the absence of the glatiramer acetate related drug substance under the same conditions, then including the batch of the glatiramer acetate related drug substance in the production of the drug product.

159. The process of claim 101, wherein step (c) further comprises i) determining the level of expression of at least one gene of ABCF2, ACP6, AFG3L2, ALMS1, ARPC4, CALMS, CCDC64, CD84, CDC6, CHAF1A, CLU, COX11, DLGAP1, DTX4, FAM49B, FHL1, FNTB, GYPC, HFE, IL10, LPHN1, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, or ZFAND6; ii) determining the level of expression of at least one gene of BIRC3, CCL24, CCR1, CISH, CSF1R, CX3CR1, CXCL10, HSPD1, ICAM1, IL1B, IFNGR1, IL27, IL2RG, IL7R, IL1RN, MMP1, MMP9, MMP14, PGRMC1, PRDM1, CARD15, CCL2, CCL5, CD14, IL10, THBD, or NFKBIA, wherein if IL1B, IL10, or MMP9 is the at least one gene selected in part (c)(ii), then selecting at least a second different gene other than IL1B, IL10, or MMP9; iii) determining the level of expression of at least one gene of C13ORF31, C14ORF10, C1ORF51, C1ORF63, CBR4, CB36, CD9, COL6A1, DAB2, GATA2, KIAA0907, LOC100506233, MCM6, MMP1, MS4A4A, MTSS1, PCMTD1, STK4, STX7, TAF15, TARP, TIA1, TMF1, TRGC2, TXNDC11, or ZCCHC7; iv) determining the level of expression of at least one gene ANXA1, ARRB2, BEAN, BIN1, C1ORF63, CD44, CD9, CFP, COL6A1, CRIP2, EPB41, Fam119a, FOR, FOXO3B, HSD11B1, HSPD1P6, LOC387790, MPEG1, MYB, OLIG1, PLD1, PPP4R2, PRDM1, RBM6, SNX27, S0D2, STATH, TARP, TREM1, TRGC2, UBN2, or ZCCHC7; v) determining the level of expression of at least one gene of ADAMS, ADAMDEC1, AKR1C2, ANXA2, ANXA2P2, ARHGAP18, ARHGAP18, ARL6IP5, ARL6IP5, ATP2C1, BID, BIRC3, BTG1, CARD15, C1ORF21, C13ORF31, C5ORF13, C5ORF32, C9ORF130, CAST, CCL2, CCL5, CD14, CD300A, CD36, CD40, CD55, CD9, CENTA2, CHST11, COL6A1, CRYBB2, CXCL10, CYLD, DAB2, EBI3, EBI2, ECOP, EGF, FABP4, FXYD2, GHRL, GIMAP8, GLIPR1, GOS2, HMGB2, HNRPLL, ICAM1, ICAM2, IFIH1, IFNGR1, IL10, IL10RA, IL411, INADL, ISG2O, ITGB5, KIAA1505, KYNU, LACTB, LOC54103, LOC388344, LOC652751, LPAAT-THETA, LPXN, MAFB, MALT1, MFI2, MGC5618, MGLL, MITE, MLF1, MMP1, MMP9, MPEG1, MTSS1, MXD1, NT5E, NFKBIE, NFKBIA, NFE2L3, NFE2L3, OSBPL11, P2RX4, P2RY5, PLEKHO1, POPDC3, PLAUR, PRDM1, PSCDBP, PTX3, RAB27B, RCSD1, RPL13, SGIP1, SLC39A8, SNORD68, SRPX2, SRA1, SLIC1, SLAMF8, SLIC1, SOD2, STATH, STEAP1, SYNJ2, SYNJ2, TATDN3, TGM5, THBD, TNFAIP3, TNFAIP6, TNFRSF9, TNFSF13B, TPSAB1, TPSB2, TREM1, TXNL2, VPS33A, or VSNL1; vi) determining the level of expression of at least one gene of ACTN4, BTBD14A, C14ORF10, CISH, CLK1, CRLF3, FAM62A, FBX045, GAPDHS, HDAC4, H1C2, HNRPD, HSPD1, LOC648342, MYB, NAPS, OXCT2, SERPINB2, SFRS14, SPFH1, STT3B, WDFY1, ZNF250, or ZNF566; vii) determining the level of expression of at least one gene of A2M, ABCB1, ABCC3, ABHD2, ACPP, ADAMDEC1, ADFP, ADORA2B, ADORA3, AHNAK, ALCAM, ANKH, ANKRD57, ANXA2, ANXA2P2, APBB1IP, AQP1, ARHGAP18, ARHGAP20, ARHGEF3, ARID5B, ARMC9, ATF5, ATP1B1, ATP6V0D2, ATP9A, ATP1OA, AYTL1, BCL2A1, BCL6, C3AR1, C13ORD31, C9ORF88, C9ORF89, C1ORF21, C1ORF22, C10ORF95, C13ORF31, C21ORF7, CARD12, CARD15, CCDC83, CCL5, CCL24, CCND1, CCR1, CD9, CD36, CD52, CD86, CD109, CD180, CD244, CDK5RAP2, CDKN1A, CENTA2, CKB, CKLF, CLEC7A, CNIH3, COL6A1, COL22A1, CRIP2, CSF1R, CSPG4, CTSL, CTSLL3, CX3CR1, CXCR7, CYBB, CYP1B1, DAB2, DAPP1, DDIT4L, DIXDC1, DOCK4, DOK2, DKFZP56400823, DKFZP68601327, EBI2, EMP1, EMR2, ENPP2, EPAS1, EPS8, EPSTI1, EVL, FABP4, FADS3, FAM26B, FGD2, FGD2, FGD4, FGL2, FN1, FTH1, GBP2, GBP3, GBP5, GCNT1, GDPD1, GNDL, GNLY, GLIPR1, GLIS3, GPC1, GPR35, H2A/R, HAVCR2, HMCN1, HIPK2, HIST2H2AA, HIVEP1, HMOX1, HSPB7, ICAM1, ID2, ID2B, 19130, 19144, IFNGR1, IGFBP3, IL2RG, IL4I1, IL1ORA, IL27RA, IL7R, IL10, INA, IRF7, ITGB5, ITGB7, KIAA15O5, KIAA1706, KMO, LBH, LFNG, LILRB1, LILRB2, LMNA, LOC51334, LOC201895, LOC284262, LOC51334, LOC643424, LOC643834, LOC643847, LOC644242, LOC645238, LOC650429, LOC650446, LOC652543, LOC653610, LOC653754, LPAAT-THETA, LPXN, MAF, MAFB, MAML2, MAML3, MARCH1, MCOLN3, MDGA1, ME1, MFI1, MFI2, MGC45491, MGLL, MITF, MMP1, MMP2, MMP9, MMP14, MMP19, MTMR11, MTSS1, MTSU1, NGEF, NME7, NPTX1, NRCAM, NRP1, NRP2, NT5E, OAS1, OLR1, P2RY5, P2RY14, PALLD, PAPSS2, PAQR5, PCDHGA1, PCDHGA2, PCDHGA3, PCDHGA4, PCDHGA5, PCDHGA6, PCDHGA7, PCDHGA8, PCDHGA9, PCDHGA10, PCDHGA11, PCDHGA12, PCDHGB1, PCDHGB2, PCDHGB3, PCDHGB4, PCDHGB5, PCDHGB7, PCDHGC3, PCDHGC4, PCDHGC5, PDK4, PDLIM7, PFKFB4, PGA5, PDLIM4, PHLDA1, PLA2G4A, PLEKHA7, PLEKHO1, POPDC3, PRDM1, PRSS23, PSCDBP, PSD3, PTAFR, PTGS1, PTPRO, PTRF, PTX3, RAB27B, RAB38, RAB7B, RAPH1, RASGRF1, RGL1, RGS13, RHBDF1, RIN2, S100A2, SART2, SERPINE2, SERTAD1, SETBP1, SGIP1, SH3TC1, SKIL, SLA, SLAMF7, SLAMF8, SLC6AS, SLC7A11, SLC12A6, 5LC37A2, SLC41A2, SLC38A6, SLC43A2, SNAI3, ST3GAL5, STATH, STEAP1, SUCNR1, SYTL1, TBXAS1, TCF4, TFAP2A, THBD, TLR4, TM7SF4, TMEM39A, TMEM158, TNCRNA, TNFSF13B, TNFRSF21, TREM1, TRIM22, TRPA1, TRPM8, TRPS1, TUBB2A, UBXD5, UGCG, UPP1, VASH1, VEGF, VSNL1, or ZFP36L1; viii) determining the level of expression of at least one gene of ABCG1, ADAMTS1, ANKRD41, ANXA3, APCDD1, BCL2, BCL11A, BMP8B, C1ORF71, C1ORF76, C1ORD121, C12ORF24, C16ORF73, C16ORF74, C20ORD27, C20ORF103, C20ORF112, CACNA2D3, CAMTA1, CAV1, CCDC85B, CDCA7L, CEBPD, CKAP4, CNTN4, COL8A2, CSPG5, CXCR4, DCUN1D4, DEPDC6, DMRT2, DUSP2, DZIP1, EBF3, EGR4, FAM117A, FKBP4, FL135848, FLOT2, GFI1, GMDS, GPR18, HAL, HNF4G, HSPC049, IL17D, IRX3, KBTBD11, KCNQ4, KCTD15, KIAA0146, KIAA0984, KIAA1026, KIAA1553, KLHL23, LGR4, LOC201164, LOC284454, LOC387763, LOC642083, LOC648232, MGC2408, MICAL1, MID1IP1, MSRB3, MUC19, NAPSB, NR1D2, PCDH8, PDE4B, PDGFD, PER2, PHF15, PKP2, PLK2, OAF, OSBPL1A, OSR2, OXCT2, PGM1, PMAIP1, PNMA6A, POU4F2, PSAT1, RAB33A, RASGRP2, RBM3B, RET, RFTN1, SERPINB2, SERPINB10, SLAIN1, SLC1A3, SLC16A1, SLC19A1, SLC27A2, SLC29A1, SLC39A14, SLCO4A1, SNF1LK, SOX12, SPFH1, SPRY1, STEAP3, SYDE2, SYNPO2, TARP, TEAD4, TDRD7, TMEM67, TPD52, TRGC2, TRGV2, TRGV9, TRIB3, TSPAN2, TUBA1, VIT, WDR49, WNT3, WT1, or YES1; ix) determining the level of expression of at least one gene of AHRR, CCDC36, CYP1B1, DOC1, EPB41L3, GAS7, GPR68, NPTX1, PDCD6, or TIPARP; x) determining the level of expression of at least one gene of ADRB2, COTL1, LOC285758, LOC644137, MALAT1, PRG1, RNF43, SAT1, THAP5, TIMP3, or TSC22D1; xi) determining the level of expression of at least one gene of AW011738, Bst2, Daxx, Gm16340, Hck, Herc6, Ifi202b, Ifi203, Ifi204, Ifi44, Ifi441, Ifit2, Inpp5b, LOC100044068, LOC100862473, Mx1, Oas11, Phf11d, Oyhin1, Sdc3, Setdb2, Tor3a, Usp18, or Zcchc2; xii) determining the level of expression of at least one gene of Ccdc711, D13ERTD608E, Fads2, Gm2a, Ifi2711, Ighm, Klk1, Scd2, Siglech, or Tspan2; xiii) determining the level of expression of at least one gene of Ahrr, AI607873, Atp10a, AW011738, Casp44, Cxc13, Gm9706, Ifi202b, Ifit2, Ifitm6, I118, Lcn2, LOC100044068, Ms4a6d, Mx1, Papd7, Rsad2, Slfn3, Slfn4, Tdrd7, Tiparp, or Zcchc2; xiv) determining the level of expression of at least one gene of Aldoc, Casp6, Ccdc711, Cox7a1, Egln3, Fam162a, Gfi1, Gpi1, Grhpr, Ifi2711, Ighm, Kcnq5, Klhdc2, Pgk1, Pkm, Tpi1, or Trappc6a; xv) determining the level of expression of at least one of 1600014C10Rik, 2810474019Rik, 6720475J19Rik, Adam8, Adar, Agrn, Ahrr, AI607873, Amigo2, Ankfy1, Apobec1, Arf4, Asb2, Ascc3, Atp10a, Atp8b4, AW011738, B4galt5, BC147527, Bst2, Casp4, Chic1, Cmpk2, Csprs, Cxc13, Cybb, Daxx, Ddit3, Ddx24, Ddx58, Ddx60, Dpp4, Eif2ak2, Emr1, Epsti1, Evi2a, Fcgr1, Fcgr4, Ftsjd2, Gcnt1, Gm11772, Gm14446, Gm15433, Gm16340, Gm20559, Gm2666, Gm7609, Gm9706, Gpnmb, Gpr15, H2-T10, H2-T9, Hck, Helz2, Herc6, Hsh2d, Hspa1b, Ifi202b, Ifi203, Ifi204, Ifi205, Ifi2712a, Ifi35, Ifi44, Ifi441, Ifih1, Ifit1, Ifit2, Ifit3, Ifitm3, I118, I17r, Inpp5b, Ins16, Irf7, Isg20, Klrk1, Lgals3 bp, Lgals9, LOC100041903, LOC100044068, LOC100503923, LOC100505160, LOC100862473, LOC664787, Lpar6, Ly6c1, Ly6c2, Mb21d1, Mitd1, Mlk1, Mmp8, Mnda, Mnda1, Ms4a4c, Ms4a6d, Mx1, Naa20, Nceh1, Ncoa7, Ngp, Nlrc5, Nmral1, Nqo1, Nt5c3, Oas1a, Oas2, Oas3, Oas11, Oas12, Ogfr, Papd7, Parp10, Parp1i, Parp12, Parp14, Phf11d, Pik3ap1, Pla2g7, Plec, Pnpt1, Ppm1k, Pydc4, Pyhin1, Ramp3, Rnf213, Rnf8, Rsad2, Rtp4, Samd91, Scin, Sdc3, Setdb2, Sgcb, Shisa5, Slco3a1, Slfn1, Slfn3, Slfn4, Slfn5, Slfn8, Slfn9, St3gal6, Tcstv3, Tdrd7, Tiparp, Tmem140, Tmem184b, Tnfsf10, Torlaip2, Tor3a, Trafd1, Trim25, Trim30a, Trim30d, Trim34a, Trim34b, Tspo, Uba7, Ubr4, Usp18, Wnt10a, Xaf1, Xaf1, Zc3hav1, Zcchc2, Zfyve26, Znfx1, or Zufsp; xvi) determining the level of expression of at least one gene of Ccdc711, D13ERTD6O8E, Fads2, Gm2a, Ifi2711, Ighm, Klk1, Scd2, Siglech, or Tspan2; xvii) determining the level of expression of at least one gene of CC12, CYBASC3, CYP1B1, FCAR, HBEGF, ID1, IL1B, 1L411, MSC, NQO1, PPP1R15A, PRDM1, SLC7A11, SRXN1, TIPARP, TMEM138, TXNRD1, or VEGF; xviii) determining the level of expression of at least one gene of BCL2, CACNA2D3, C13ORF18, C20ORF103, C5ORF13, CDCA7, DEPDC6, GATM, HAL, HSPA1A, HSPC049, LOC645919, LRMP, OAF, POU4F2, RASGRP2, RET, SERPINB2, SERPINB8, SPFH1, or TDRD7; xix) determining the level of expression of at least one gene of ABCC1, ABHD12, ABHD5, ACPP, ACSL1, ADFP, ADORA2B, ADORA3, AHRR, AKNA, AKR1C1, AKR1C2, AKR1C3, ALAS1, ALOX5AP, ANKRD57, ANXA2, APBB1IP, APRIN, ARHGAP20, ARHGEF3, ARRB2, ARRDC4, ASB2, ATF5, ATG7, ATP6V0B, ATP6V0C, ATP9A, ATP9B, AXL, AYTL1, BCL2A1, BCL3, BCL6, BHLHB2, BTG1, BTG2, BTG3, C10ORF22, C10ORF54, C10ORF56, C12ORF35, C13ORF31, C14ORF43, C15ORF39, C17ORF32, C19ORF58, C1ORF122, C1ORF144, C10RF162, C1ORF21, C1ORF38, C3AR1, C5ORF20, C6ORF166, C9ORF16, C9ORF88, CALN1, CARD15, CCL2, CCL5, CCND3, CCNL1, CCR1, CD109, CD244, CD300A, CD40, CD44, CD83, CD9, CDCA4, CDK5RAP2, CDKN1A, CHST11, CIDEC, CKB, CLEC5A, CLEC7A, CMTM3, CPEB2, CPEB4, CSF1R, CSGLCA-T, CSGLCA-T, CSPG2, CSPG2, CTSB, CTSH, CUTL1, CXCL1, CXCL2, CXXC5, CYBASC3, CYBB, CYLD, CYLD, CYP1B1, DDB1, DGAT2, DKFZP686O1327, DOC, DOK2, DUSP6, EBI2, ECGF1, ECOP, EFHD2, EIF1, ELL2, ELOVL1, EMP2, EMR2, EPAS1, EPB41L3, EPB41L3, EXT1, F3, FADS3, FAM100B, FCAMR, FCAR, FGD3, FGD4, FGL2, FLJ20489, FLJ20701, FLJ90013, FLRT2, FPRL1, FTH1, FUCA1, GAS7, GCNT1, GNA15, GPR35, GPR6B, GSR, GSR, H2A, HBEGF, HERPUD1, HIPK2, HIST2H2AA, HIVEP1, HMOX1, HNRPLL, HPCAL1, ID1, 102, IER5, 1F130, IFNGR1, IFNGR2, IGFBP3, IL1ORA, IL1B, IL1R1, IL1RN, IL1RN, IL21R, IL27RA, IL27RA, IL4I1, IL4R, IRF5, ITGB7, JDP2, JUN, JUNB, JUND, KCNN4, KIAA0247, KIAA0999, KIAA1505, KIAA17O6, KIAA1913, KITLG, KLF13, KLF4, KLF6, KLHL18, LACTB, LAT, LHX2, LOC113179, LOC338758, LOC440934, LOC54103, LOC644242, LOC648998, LOC650429, LOC650446, LOC651816, LOC653524, LOC653361, LOC653840, LOC653361, LOC653506, LOC653610, LOC653840, LOC653626, LPAAT-THETA, LPL, LPXN, LRG1, LRP10, MAFB, MAFF, MALT1, MAML2, MAP1LC3B, MARCKSL1, MBP, MCL1, ME1, METRNL, MGAT4A, MGC13379, MGLL, MMP2, MMP9, MOBKL2A, MSC, MST150, MTF1, MTUS1, MYH10, NAB1, NCF1, NCF2, NCF4, NEU1, NFE2L3, NFKB1, NFKB2, NFKBIA, NFKBIE, NFXL1, NINJ1, NOTCH1, NOTCH2NL, NPTX1, NQO1, NRP1, NRP2, NT5E, NUAK1, P2RY5, P2RY6, PACSIN2, PDCD6, PDK4, PDLIM4, PECAM1, PEX19, POD, PHLDA1, PHLDA2, PIK3R5, PIR, PITPNA, PKM2, PLAU, PLAUR, PLEKHO1, PNKD, POPDC3, PPIF, PPP1R15A, PRDM1, PRKCA, PSCD4, PSCDBP, PSMD1, PTAFR, PTGS1, PTPN14, PTPRE, PTX3, QPRT, RAB13, RAB27B, RAB38, RAB6IP1, RAI17, RAP2B, RAPGEF1, RCN1, RELB, RGL1, RGS1, RGS2, RIN3, RIT1, RND3, RSNL2, RSPO3, RUNX3, SAMSN1, SAP30, SASH1, SAT1, SDC4, SEMA4C, SERPINE2, SERTAD1, SFRS7, SGK, SH3GL1, SH3TC1, SLAMF8, SLC15A3, SLC16A3, SLC20A1, SLC23A2, SLC25A14, SLC25A19, SLC25A20, SLC2A1, SLC2A6, SLC37A2, SLC39A8, SLC43A2, SLC45A3, SLC4A2, SLC4A5, SLC6A6, SLC7A11, SLC7A11, SLIC1, SMOX, SNAI3, SOD2, SPRY2, SPSB1, SQRDL, SQSTM1, SRXN1, SSH1, ST3GAL5, STAT1, STK40, TFAP2A, TFDP1, TFEB, TGIF, THBD, TIPARP, TMEM138, TMTC1, TNFAIP3, TNFAIP6, TNFAIP8L1, TNFRSF10D, TNFRSF1B, TNFRSF21, TNFSF13B, TNFSF7, TP53BP2, TRAF3, TRAF3IP2, TRIB1, TRIB3, TRIM16, TRIM16L, TRPA1, TRPS1, TRPS1, TSHZ3, TTLL4, TXNRD1, UBE2S, UGCG, ULBP2, UPP1, URP2, VASH1, VEGF, VSNL1, YRDC, ZBTB24, ZCCHC10, ZFAND5, ZFP36L1, ZNF366, ZNF516, or ZNF697; xx) determining the level of expression of at least one gene of ABHD14B, ACTN1, ACY1L2, ADA, ADD2, AFF1, AIG1, AK2, AKAP1, ALS2CR13, ANKRD45, ANKRD55, APPL, ARHGEF6, ATG16L2, ATP8B3, ATP8B4, ATPBD1C, B3GNT7, BCL11A, BCL2, BMP8B, BRE, BSPRY, BTBD14A, C13ORF18, C13ORF18, C14ORF106, C15ORF41, C16ORF73, C1ORF121, C1ORF63, C1QBP, C1S, C20ORF103, C20ORF112, C20ORF12, C3ORF14, C5ORF13, C6ORF147, C7ORF24, C9ORF103, CABC1, CACNA2D3, CACYBP, CALCOCO2, CAMSAP1L1, CAMSAP1L1, CAT, CAV1, CDCA7, CERKL, CHST12, CHST5, CITED4, CLINT1, CLSTN2, CLTCL1, CNTN4, COL4A1, COL5A2, CUGBP2, CXORF21, DAB1, DENND4A, DEPDC6, DHRS9, DMRT2, DUT, EIF4A2, ESD, FLJ12078, FLJ20152, FLJ23861, FLJ36166, FOXP1, GATM, GGA2, GOLGA1, GOLGA8C, GOLGA8D, GOLGA8E, GOLGA8F, GOLGA8G, GPD1L, GPR18, HADH, HAL, HDAC9, HGF, HIG2, HISPPD1, HNRPA3, HNRPH3, HOXB3, HSPA1A, HSPA4L, HSPB1, HSPC049, 102, ID2B, IDH1, IDH1, IHPK2, IRX3, ITGA4, KBTBD11, KCNN2, KIAA0960, KLF10, LARS, LGR4, LIMA1, LIX1L, LOC129285, LOC148203, LOC197322, LOC203274, LOC220594, LOC254559, LOC284702, LOC285084, LOC285758, LOC340061, LOC340061, LOC388189, LOC474170, LOC643458, LOC645919, LOC646456, LOC90835 LONRF1, LRMP, LYST, MACF1, MDH1, METTL7B, METTLE, MICAL1, MLSTD1, MNDA, MRPL24, MS4A3, MS4A4A, MS4A6A, MS4A7, MSRB3, MT1E, MT1H, MT1M, MTBP, MTHFD1, MTL5, MTR, MUC19, MUM1, MYADM, NAPSB, NAPSB, NAT11, NOC2L, NPAL3, OAF, OCRL, OMA1, OSBPL1A, OXCT2, PDCD4, PHACTR3, PHYH, PIGM, PIWIL4, PNMA6A, POU4F2, PRKAB2, PRLR, PSAT1, PSAT1, PTGER3, PTPLAD2, RABGAP1L, RAD17, RASGRP2, RBKS, RET, RNASEH2B, RNASET2, SELPLG, SERPINB10, SERPINB2, SERPINB8, SERPINI2, SKAP2, SLAIN1, SLC16A4, SLC22A15, SLC22A16, SLC40A1, SMARCA2, SNAPC3, SNX10, SPFH1, SPTBN1, ST3GAL3, STAR, STREP, SYNPO2, TADA1L, TCFL5, TDRD7, THTPA, TIFA, TLE1, TMEM14A TOP2B, TPD52, TPM1, TRAF3IP3, TSPAN2, TTC9C, UBE2B, UBP1, UHRF2, VLDLR, VPS35, WASF1, WDFY1, WDR49, WDR68, WHDC1L1, WHDC1L2, ZBTB33, ZBTB44, ZF, ZNF207, ZNF519, ZNF658, or ZNF92; xxi) determining the level of expression of at least one gene of CCL2, CCL5, CXCL10, IL1RN, or MMP9; xxii) determining the level of expression of at least one gene of CCL5, CXCL10, or MMP9; xxiii) determining the level of expression of at least one gene of IL10 or CCL2; xxiv) determining the level of expression of at least one gene of IFNg, TNF, CCL3, CXCL8, or IL-10; xxv) determining the level of expression of at least one gene of MMP9, CCL2, CCL5, CXCL1, or IL1B; xxvi) determining the level of expression of at least one gene of MMP9, CXCL10, CCL2, or CCL5; xxix) determining the level of expression of at least one gene of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, or MIF; or xxx) determining the level of expression of at least one gene of CCL3, MMP9, CCL22, CCL24, CX3CL1, CCL20, CCL2, TNF, 1L8, CCL13, CCL5, IL1B, CCL8, IL10, CXCL11, CXCL13, CXCL10, CCL7, CCL1, CXCL1, IFNg, CCL26, MIF, IL16, IL6, CCL25, IL2, CCL19, CXCL2, CXCL9, or CXCL5.

160. The process of claim 159, wherein if the level of expression of ABI2 is upregulated relative to the level of expression by the same type of cells in the absence of the glatiramer acetate related drug substance under the same conditions then
a. i) including the batch of the glatiramer acetate related drug substance in the production of the drug product if the level of expression of one or more genes selected from the group consisting of ABCF2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, IL10, LPHN1, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, and ZFAND6 is not substantially identical to the level of expression by the same type of cells in the absence of the glatiramer acetate related drug substance under the same conditions; ii) including the batch of the glatiramer acetate related drug substance in the production of the drug product if the level of expression of one or more genes selected from the group consisting of BIRC3, CCL24, CCR1, CISH, CSF1R, CX3CR1, CXCL10, HSPD1, ICAM1, IL1B, IFNGR1, IL27, IL2RG, IL7R, IL1RN, MMP1, MMP9, MMP14, PGRMC1, PRDM1, CARD15, CCL2, CCL5, CD14, IL10, THBD, and NFKBIA is substantially identical to the level of expression by the same type of cells in the presence of the glatiramer acetate drug substance under the same conditions; iii) including the batch of the glatiramer acetate related drug substance in the production of the drug product if the level of expression of one or more genes selected from the group consisting of C13ORF31, C14ORF10, C1ORF51, C1ORF63, CBR4, CB36, CD9, COL6A1, DAB2, GATA2, KIAA0907, LOC100506233, MCM6, MMP1, MS4A4A, MTSS1, PCMTD1, STK4, STX7, TAF15, TARP, TIA1, TMF1, TRGC2, TXNDC11, and ZCCHC7 is substantially identical to the level of expression by the same type of cells in the presence of the glatiramer acetate drug substance under the same conditions; iv) including the batch of the glatiramer acetate related drug substance in the production of the drug product it the level of expression of one or more genes selected from the group consisting of ANXA1, ARRB2, BEAN, BIN1, C1ORF63, CD44, CD9, CFP, COL6A1, CRIP2, EPB41, Fam119a, FGR, FOXO3B, HSD11B1, HSPD1P6, LOC387790, MPEG1, MYB, OLIG1, PLD1, PPP4R2, PRDM1, RBM6, SNX27, S0D2, STATH, TARP, TREM1, TRGC2, UBN2, and ZCCHC7 is substantially identical to the level of expression by the same type of cells in the presence of the glatiramer acetate drug substance under the same conditions; v) including the batch of the glatiramer acetate related drug substance in the production of the drug product if the level of expression of one or more genes selected from the group consisting of ADAM9, ADAMDEC1, AKR1C2, ANXA2, ANXA2P2, ARHGAP18, ARHGAP18, ARL6IP5, ARL6IP5, ATP2C1, BID, BIRC3, BTG1, CARD15, C1ORF21, C13ORF31, C5ORF13, C5ORF32, C9ORF13O, CAST, CCL2, CCL5, CD14, CD300A, CD36, CD40, CD55, CD9, CENTA2, CHST11, COL6A1, CRYBB2, CXCL10, CYLD, DAB2, EBI3, EBI2, ECOP, EGF, FABP4, FXYD2, GHRL, GIMAP8, GLIPR1, CD82, HMGB2, HNRPLL, ICAM1, ICAM2, IFIH1, IFNGR1, IL10, IL10RA, IL411, INADL, ISG2O, ITGB5, KIAA1505, KYNU, LACTB, LOC54103, LOC388344, LOC652751, LPAAT-THETA, LPXN, MAFB, MALT1, MFI2, MGC5618, MGLL, MITF, MLF1, MMP1, MMP9, MPEG1, MTSS1, MXD1, NT5E, NFKBIE, NFKBIA, NFE2L3, NFE2L3, OSBPL11, P2RX4, P2RY5, PLEKHO1, POPDC3, PLAUR, PRDM1, PSCDBP, PTX3, RAB27B, RCSD1, RPL13, SGIP1, SLC39A8, SNORD6B, SRPX2, SRA1, SLIC1, SLAMF8, SLIC1, SOD2, STATH, STEAP1, SYNJ2, SYNJ2, TATDN3, TGM5, THBD, TNFAIP3, TNFAIP6, TNFRSF9, TNFSF13B, TPSAB1, TPSB2, TREM1, TXNL2, VPS33A, and VSNL1 is substantially identical to the level of expression by the same type of cells in the presence of the glatiramer acetate drug substance under the same conditions; vi) including the batch of the glatiramer acetate related drug substance in the production of the drug product if the level of expression of one or more genes selected from the group consisting of ACTN4, BTBD14A, C14ORF10, CISH, CLK1, CRLF3, FAM62A, FBX045, GAPDHS, HDAC4, HIC2, HNRPD, HSPD1, LOC648342, MYB, NAPB, OXCT2, SERPINB2, SFRS14, SPFH1, STT3B, WDFY1, ZNF250, and ZNF566, is substantially identical to the level of expression by the same type of cells in the presence of the glatiramer acetate drug substance under the same conditions; vii) including the batch of the glatiramer acetate related drug substance in the production of the drug product if the level of expression of one or more genes selected from the group consisting of A2M, ABCB1, ABCC3, ABHD2, ACPP, ADAMDEC1, ADFP, ADORA2B, ADORA3, AHNAK, ALCAM, ANKH, ANKRD57, ANXA2, ANXA2P2, APBB1IP, AQP1, ARHGAP18, ARHGAP20, ARHGEF3, ARID5B, ARMC9, ATF5, ATP1B1, ATP6V0D2, ATP9A, ATP10A, AYTL1, BCL2A1, BCL6, C3AR1, C13ORD31, C9ORF88, C9ORF89, C1ORF21, C1ORF21, C10ORF95, C13ORF31, C21ORF7, CARD12, CARD15, CCDC83, CCL5, CCL24, CCND1, CCR1, CD9, CD36, CD52, CD86, CD109, CD180, CD244, CDK5RAP2, CDKN1A, CENTA2, CKB, CKLF, CLEC7A, CNIH3, COL6A1, COL22A1, CRIP2, CSF1R, CSPG4, CTSL, CTSLL3, CX3CR1, CXCR7, CYBB, CYP1B1, DAB2, DAPP1, DDIT4L, DIXDC1, DOCK4, DOK2, DKFZP56400823, DKFZP68601327, E312, EMP1, EMR2, ENPP2, EPAS1, EPS8, EPSTI1, EVL, FABP4, FADS3, FAM26B, FGD2, FGD2, FGD4, FGL2, FN1, FTH1, GBP2, GBP3, GBP5, GCNT1, CD801, GNDL, ONLY, GLIPR1, GLIS3, 0801, GPR35, H2A/R, HAVCR2, HMCN1, HIPK2, HIST2H2AA, HIVEP1, HMOX1, HSPB7, ICAM1, 102, ID2B, IFI30, IFI44, IFNGR1, IGFBP3, IL2RG, IL4I1, IL1ORA, IL27RA, IL7R, IL1O, INA, IRF7, ITGB5, ITGB7, KIAA1505, KIAA1706, KMO, LBH, LFNG, LILRB1, LILRB2, LMNA, LOC51334, LOC201895, LOC284262, LOC51334, LOC643424, LOC643834, LOC643847, LOC644242, LOC645238, LOC650429, LOC650446, LOC652543, LOC653610, LOC653754, LPAAT-THETA, LPXN, MAF, MAFB, MAML2, MAML3, MARCH1, MCOLN3, MDGA1, ME1, MFI1, MFI2, MGC45491, MGLL, MITF, MMP1, MMP2, MMP9, MMP14, MMP19, MTMR11, MTSS1, MTSU1, NGEF, NME7, NPTX1, NRCAM, NRP1, NRP2, NT5E, OAS1, OLR1, P2RY5, P2RY14, PALLD, PAPSS2, PAQR5, PCDHGA1, PCDHGA2, PCDHGA3, PCDHGA4, PCDHGA5, PCDHGA6, PCDHGA7, PCDHGA8, PCDHGA9, PCDHGA10, PCDHGA11, PCDHGA12, PCDHGB1, PCDHGB2, PCDHGB3, PCDHGB4, PCDHGB5, PCDHGB7, PCDHGC3, PCDHGC4, PCDHGC5, PDK4, PDLIM7, PFKFB4, PGA5, PDLIM4, PHLDA1, PLA2G4A, PLEKHA7, PLEKHO1, POPDC3, PRDM1, PRSS23, PSCDBP, PSD3, PTAFR, PTGS1, PTPRO, PTRF, PTX3, RAB27B, RAB38, RAB7B, RAPH1, RASGRF1, RGL1, RGS13, RHBDF1, RIN2, S100A2, SART2, SERPINE2, SERTAD1, SETBP1, SGIP1, SH3TC1, SKIL, SLA, SLAMF7, SLAMF8, SLC6AS, SLC7A11, SLC12A6, 5LC37A2, SLC41A2, SLC38A6, SLC43A2, SNAI3, ST3GAL5, STATH, STEAP1, SUCNR1, SYTL1, TBXAS1, TCF4, TFAP2A, THBD, TLR4, TM7SF4, TMEM39A, TMEM158, TNCRNA, TNFSF13B, TNFRSF21, TREM1, TRIM22, TRPA1, TRPM8, TRPS1, TUBB2A, UBXD5, UGCG, UPP1, VASH1, VEGF, VSNL1, and ZFP36L1, is upregulated to the level of expression by the same type of cells in the absence of the glatiramer acetate related drug substance under the same conditions; viii) including the batch of the glatiramer acetate related drug substance in the production of the drug product if the level of expression of one or more genes selected from the group consisting of ABCG1, ADAMTS1, ANKRD41, ANXA3, APCDD1, BCL2, BCL11A, BMP5B, C1ORF71, C1ORF76, C1ORD121, C12ORF24, C16ORF73, C16ORF74, C20ORD27, C20ORF103, C20ORF112, CACNA2D3, CAMTA1, CAV1, CCDC85B, CDCA7L, CEBPD, CKAP4, CNTN4, COL8A2, CSPG5, CXCR4, DCUN1D4, DEPDC6, DMRT2, DUSP2, DZIP1, EBF3, EGR4, FAM117A, FKBP4, FL135848, FLOT2, GFI1, GMDS, GPR18, HAL, HNF4G, HSPC049, IL17D, IRX3, KBTBD11, KCNQ4, KCTD15, KIAA0146, KIAA0984, KIAA1026, KIAA1553, KLHL23, LGR4, LOC201164, LOC284454, LOC387763, LOC642083, LOC648232, MGC2408, MICAL1, MID1IP1, MSRB3, MUC19, NAPSB, NR1D2, PCDH8, PDE4B, PDGFD, PER2, PHF15, PKP2, PLK2, OAF, OSBPL1A, OSR2, OXCT2, PGM1, PMAIP1, PNMA6A, POU4F2, PSAT1, RAB33A, RASGRP2, RBM38, RET, RFTN1, SERPINB2, SERPINB10, SLAIN1, SLC1A3, SLC16A1, SLC19A1, SLC27A2, SLC29A1, SLC39A14, SLCO4A1, SNF1LK, SOX12, SPFH1, SPRY1, STEAP3, SYDE2, SYNPO2, TARP, TEAD4, TDRD7, TMEM67, TPD52, TRGC2, TRGV2, TRGV9, TRIB3, TSPAN2, TUBA1, VIT, WDR49, WNT3, WT1, and YES1, is downregulated to the level of expression by the same type of cells in the absence of the glatiramer acetate related drug substance under the same conditions; or ix) including the batch of the glatiramer acetate related drug substance in the production of the drug product if the level of expression of one or more genes selected from the group consisting of AHRR, CCDC36, CYP1B1, DOC1, EPB41L3, GAS7, GPR68, NPTX1, PDCD6, and TIPARP is substantially identical to the level of expression by the same type of cells in the presence of the glatiramer acetate drug substance under the same conditions; x) including the batch of the glatiramer acetate related drug substance in the production of the drug product if the level of expression of one or more genes selected from the group consisting of ADRB2, COTL1, LOC285758, LOC644137, MALAT1, PRG1, RNF43, SAT1, THAP5, TIMP3, and TSC22D1 is substantially identical to the level of expression by the same type of cells in the presence of the glatiramer acetate drug substance under the same conditions; xi) including the batch of the glatiramer acetate related drug substance in the production of the drug product if the level of expression of one or more genes selected from the group consisting of AW011738, Bst2, Daxx, Gm16340, Hck, Herc6, Ifi202b, Ifi203, Ifi204, Ifi44, Ifi441, Ifit2, Inpp5b, LOC100044068, LOC100862473, Mx1, Oas11, Phf11d, Oyhin1, Sdc3, Setdb2, Tor3a, Usp18, and Zcchc2 is substantially identical to the level of expression by the same type of cells in the presence of the glatiramer acetate drug substance under the same conditions; xii) including the batch of the glatiramer acetate related drug substance in the production of the drug product if the level of expression of one or more genes selected from the group consisting of Ccdc711, D13ERTD608E, Fads2, Gm2a, Ifi2711, Ighm, Klk1, Scd2, Siglech, and Tspan2 is substantially identical to the level of expression by the same type of cells in the presence of the glatiramer acetate drug substance under the same conditions; xiii) including the batch of the glatiramer acetate related drug substance in the production of the drug product if the level of expression of one or more genes selected from the group consisting of Ahrr, AI607873, Atp10a, AW011738, Casp44, Cxc13, Gm9706, Ifi202b, Ifit2, Ifitm6, I118, Lcn2, LOC100044068, Ms4a6d, Mx1, Papd7, Rsad2, Slfn3, Slfn4, Tdrd7, Tiparp, and Zcchc2 is substantially identical to the level of expression by the same type of cells in the presence of the glatiramer acetate drug substance under the same conditions; xiv) including the batch of the glatiramer acetate related drug substance in the production of the drug product if the level of expression of one or more genes selected from the group consisting of Aldoc, Casp6, Ccdc711, Cox7a1, Egln3, Fam162a, Gfi1, Gpi1, Grhpr, Ifi2711, Ighm, Kcnq5, Klhdc2, Pgk1, Pkm, Tpi1, and Trappc6a is substantially identical to the level of expression by the same type of cells in the presence of the glatiramer acetate drug substance under the same conditions; xv) including the batch of the glatiramer acetate related drug substance in the production of the drug product if the level of expression of one or more genes selected from the group consisting of 1600014C10Rik, 2810474019Rik, 6720475J19Rik, Adam8, Adar, Agrn, Ahrr, A1607873, Amigo2, Ankfy1, Apobec1, Arf4, Asb2, Ascc3, Atp10a, Atp8b4, AW011738, B4galt5, BC147527, Bst2, Casp4, Chic1, Cmpk2, Csprs, Cxc13, Cybb, Daxx, Ddit3, Ddx24, Ddx58, Ddx60, Dpp4, Eif2ak2, Emr1, Epsti1, Evi2a, Fcgr1, Fcgr4, Ftsjd2, Gcnt1, Gm11772, Gm14446, Gm15433, Gm16340, Gm20559, Gm2666, Gm7609, Gm9706, Gpnmb, Gpr15, H2-T10, H2-T9, Hck, Helz2, Herc6, Hsh2d, Hspa1b, Ifi202b, Ifi203, Ifi204, Ifi205, Ifi2712a, Ifi35, Ifi44, Ifi44l, Ifih1, Ifit1, Ifit2, Ifit3, Ifitm3, I118, I17r, Inpp5b, Ins16, Irf7, Isg20, Lgals3 bp, Lgals9, LOC100041903, LOC100044068, LOC100503923, LOC100505160, LOC100862473, LOC664787, Lpar6, Ly6c1, Ly6c2, Mb21d1, Mitd1, Mlk1, Mmp8, Mnda, Mnda1, Ms4a4c, Ms4a6d, Mx1, Naa20, Nceh1, Ncoa7, Ngp, Nlrc5, Nmral1, Nqo1, Nt5c3, Oas1a, Oas2, Oas3, Oas11, Oas12, Ogfr, Papd7, Parp10, Parp11, Parp12, Parp14, Phf11d, Pik3ap1, Pla2g7, Plec, Pnpt1, Ppm1k, Pydc4, Pyhin1, Ramp3, Rnf213, Rnf8, Rsad2, Rtp4, Samd91, Scin, Sdc3, Setdb2, Sgcb, Shisa5, Slco3a1, Slfn1, Slfn3, Slfn4, Slfn5, Slfn8, Slfn9, St3gal6, Tcstv3, Tdrd7, Tiparp, Tmem140, Tmem184b, Tnfsf10, Torlaip2, Tor3a, Trafd1, Trim25, Trim30a, Trim30d, Trim34a, Trim34b, Tspo, Uba7, Ubr4, Usp18, Wnt10a, Xaf1, Xaf1, Zc3hav1, Zcchc2, Zfyve26, Znfx1, and Zufsp is substantially identical to the level of expression by the same type of cells in the presence of the glatiramer acetate drug substance under the same conditions; xvi) including the batch of the glatiramer acetate related drug substance in the production of the drug product if the level of expression of one or more genes selected from the group consisting of Ccdc711, D13ERTD608E, Fads2, Gm2a, Ifi2711, Ighm, Klk1, Scd2, Siglech, and Tspan2 is substantially identical to the level of expression by the same type of cells in the presence of the glatiramer acetate drug substance under the same conditions; xvii) including the batch of the glatiramer acetate related drug substance in the production of the drug product if the level of expression of one or more genes selected from the group consisting of CC12, CYBASC3, CYP1B1, FCAR, HBEGF, ID1, IL1B, IL411, MSC, N(201, PPP1R15A, PRDM1, SLC7A11, SRXN1, TIPARP, TMEM138, TXNRD1, and VEGF is substantially identical to the level of expression by the same type of cells in the presence of the glatiramer acetate drug substance under the same conditions; xviii) including the batch of the glatiramer acetate related drug substance in the production of the drug product if the level of expression of one or more genes selected from the group consisting of BCL2, CACNA2D3, C13ORF18, C20ORF103, C5ORF13, CDCA7, DEPDC6, GATM, HAL, HSPA1A, HSPC049, LOC645919, LRMP, OAF, POU4F2, RASGRP2, RET, SERPINB2, SERPINB8, SPFH1, and TDRD7 is substantially identical to the level of expression by the same type of cells in the presence of the glatiramer acetate drug substance under the same conditions; xix) including the batch of the glatiramer acetate related drug substance in the production of the drug product if the level of expression of one or more genes selected from the group consisting of ABCC1, ABHD12, ABHD5, ACPP, ACSL1, ADFP, ADORA2B, ADORA3, AHRR, AKNA, AKR1C1, AKR1C2, AKR1C3, ALAS1, ALOX5AP, ANKRD57, ANXA2, APBB1IP, APRIN, ARHGAP20, ARHGEF3, ARRB2, ARRDC4, ASB2, ATF5, ATG7, ATP6V0B, ATP6V0C, ATP9A, ATP9B, AXL, AYTL1, BCL2A1, BCL3, BCL6, BHLHB2, BTG1, BTG2, BTG3, C10ORF22, C10ORF54, C10ORF56, C12ORF35, C13ORF31, C14ORF43, C15ORF39, C17ORF32, C19ORF58, C1ORF122, C1ORF144, C1ORF162, C1ORF21, C1ORF38, C3AR1, C5ORF20, C6ORF166, C9ORF16, C9ORF88, CALN1, CARD15, CCL2, CCL5, CCND3, CCNL1, CCR1, CD1O9, CD244, CD300A, C040, CD44, CD83, CD9, CDCA4, CDK5RAP2, CDKN1A, CHST11, CIDEC, CKB, CLEC5A, CLEC7A, CMTM3, CPEB2, CPEB4, CSF1R, CSGLCA-T, CSGLCA-T, CSPG2, CSPG2, CTSB, CTSH, CUTL1, CXCL1, CXCL2, CXXC5, CYBASC3, CYBB, CYLD, CYLD, CYP1B1, DDB1, DGAT2, DKFZP68601327, DOC1, DOK2, DUSP6, EBI2, ECGF1, ECOP, EFHD2, EIF1, ELL2, ELOVL1, EMP2, EMR2, EPAS1, EPB41L3, EPB41L3, EXT1, F3, FADS3, FAM100B, FCAMR, FCAR, FGD3, FGD4, FGL2, FLJ20489, FLJ20701, FLJ90013, FLRT2, FPRL1, FTH1, FUCA1, GAS7, GCNT1, GNA15, GPR35, GPR68, GSR, GSR, H2A, HBEGF, HERPUD1, HIPK2, HIST2H2AA, HIVEP1, HMOX1, HNRPLL, HPCAL1, ID1, 102, IER5, IFI30, IFNGR1, IFNGR2, IGFBP3, IL10RA, IL1B, IL1R1, IL1RN, IL1RN, IL21R, IL27RA, IL27RA, 1L411, IL4R, IRF5, ITGB7, JDP2, JUN, JUNB, JUND, KCNN4, KIAA0247, KIAA0999, KIAA1505, KIAA17O6, KIAA1913, KITLG, KLF13, KLF4, KLF6, KLHL18, LACTB, LAT, LHX2, LOC113179, LOC338758, LOC440934, LOC54103, LOC644242, LOC648998, LOC650429, LOC650446, LOC651816, LOC653524, LOC653361, LOC653840, LOC653361, LOC653506, LOC653610, LOC653840, LOC653626, LPAAT-THETA, LPL, LPXN, LRG1, LRP10, MAFB, MAFF, MALT1, MAML2, MAP1LC3B, MARCKSL1, MBP, MCL1, ME1, METRNL, MGAT4A, MGC13379, MGLL, MMP2, MMP9, MOBKL2A, MSC, MST150, MTF1, MTUS1, MYH10, NAB1, NCF1, NCF2, NCF4, NEU1, NFE2L3, NFKB2, NFKBIA, NFKBIE, NFXL1, NINJ1, NOTCH1, NOTCH2NL, NPTX1, NQO1, NRP1, NRP2, NT5E, NUAK1, P2RY5, P2RY6, PACSIN2, PDCD6, PDK4, PDLIM4, PECAM1, PEX19, PGD, PHLDA1, PHLDA2, PIK3R5, PIR, PITPNA, PKM2, PLAU, PLAUR, PLEKHO1, PNKD, POPDC3, PPIF, PPP1R15A, PRDM1, PRKCA, PSCD4, PSCDBP, PSMD1, PTAFR, PTGS1, PTPN14, PTPRE, PTX3, QPRT, RAB13, RAB27B, RAB38, RAB6IP1, RAI17, RAP2B, RAPGEF1, RCN1, RELB, RGL1, RGS1, RGS2, RIN3, RIT1, RND3, RSNL2, RSPO3, RUNX3, SAMSN1, SAP30, SASH1, SAT1, SDC4, SEMA4C, SERPINE2, SERTAD1, SFRS7, SGK, SH3GL1, SH3TC1, SLAMF8, SLC15A3, SLC16A3, SLC20A1, SLC23A2, SLC25A14, SLC25A19, SLC25A20, SLC2A1, SLC2A6, SLC37A2, SLC39A8, SLC43A2, SLC45A3, SLC4A2, SLC4A5, SLC6A6, SLC7A11, SLC7A11, SLIC1, SMOX, SNAI3, SOD2, SPRY2, SPSB1, SQRDL, SQSTM1, SRXN1, SSH1, ST3GAL5, STAT1, STK40, TFAP2A, TFDP1, TFEB, TGIF, THBD, TIPARP, TMEM138, TMTC1, TNFAIP3, TNFAIP6, TNFAIP8L1, TNFRSF10D, TNFRSF1B, TNFRSF21, TNFSF13B, TNFSF7, TP53BP2, TRAF3, TRAF3IP2, TRIB1, TRIB3, TRIM16, TRIM16L, TRPA1, TRPS1, TRPS1, TSHZ3, TTLL4, TXNRD1, UBE2S, UGCG, ULBP2, UPP1, URP2, VASH1, VEGF, VSNL1, YRDC, ZBTB24, ZCCHC10, ZFAND5, ZFP36L1, ZNF366, ZNF516, and ZNF697 is substantially identical to the level of expression by the same type of cells in the presence of the glatiramer acetate drug substance under the same conditions; xx) including the batch of the glatiramer acetate related drug substance in the production of the drug product if the level of expression of one or more genes selected from the group consisting of ABHD14B, ACTN1, ACY1L2, ADA, ADD2, AFF1, AIG1, AK2, AKAP1, ALS2CR13, ANKRD45, ANKRD55, APPL, ARHGEF6, ATG16L2, ATP8B3, ATP8B4, ATPBD1C, B3GNT7, BCL11A, BCL2, BMP8F, BRE, BSPRY, BTBD14A, C13ORF18, C13ORF18, C14ORF106, C15ORF41, C16ORF73, C1ORF121, C1ORF63, C1QBP, CIS, C20ORF103, C20ORF112, C20ORF12, C3ORF14, C5ORF13, C6ORF147, C7ORF24, C9ORF103, CABC1, CACNA2D3, CACYBP, CALCOCO2, CAMSAP1L1, CAMSAP1L1, CAT, CAV1, CDCA7, CERKL, CHST12, CHST5, CITED4, CLINT1, CLSTN2, CLTCL1, CNTN4, COL4A1, COL8A2, CUGBP2, CXORF21, DAB1, DENND4A, DEPDC6, DHRS9, DMRT2, DUT, EIF4A2, ESD, FLJ12078, FLJ20152, FLJ23861, FLJ36166, FOXP1, GATM, GGA2, GOLGA1, GOLGA8C, GOLGA8D, GOLGA8E, GOLGA8F, GOLGA8G, GPD1L, GPR18, HADH, HAL, HDAC9, HGF, HIG2, HISPPD1, HNRPA3, HNRPH3, HOXB3, HSPA1A, HSPA4L, HSPB1, HSPC049, 102, ID2B, IDH1, IDH1, IHPK2, IRX3, ITGA4, KBTBD11, KCNN2, KIAA0960, KLF1O, LARS, LGR4, LIMA1, LIX1L, LOC129285, LOC148203, LOC197322, LOC203274, LOC220594, LOC254559, LOC284702, LOC285084, LOC285758, LOC340061, LOC340061, LOC388189, LOC474170, LOC643458, LOC645919, LOC646456, LOC90835 LONRF1, LRMP, LYST, MACF1, MDH1, METTL7B, METTLE, MICAL1, MLSTD1, MNDA, MRPL24, MS4A3, MS4A4A, MS4A6A, MS4A7, MSRB3, MT1E, MT1H, MT1M, MTBP, MTHFD1, MTL5, MTR, MUC19, MUM1, MYADM, NAPSB, NAPSB, NAT11, NOC2L, NPAL3, OAF, OCRL, OMA1, OSBPL1A, OXCT2, PDCD4, PHACTR3, PHYH, PIGM, PIWIL4, PNMA6A, POU4F2, PRKAB2, PRLR, PSAT1, PSAT1, PTGER3, PTPLAD2, RABGAP1L, RAD17, RASGRP2, RBKS, RET, RNASEH2B, RNASET2, SELPLG, SERPINB10, SERPINB2, SERPINB8, SERPINI2, SKAP2, SLAIN1, SLC16A4, SLC22A15, SLC22A16, SLC40A1, SMARCA2, SNAPC3, SNX10, SPFH1, SPTBN1, ST3GAL3, STAR, STRBP, SYNPO2, TADA1L, TCFL5, TDRD7, THTPA, TIFA, TLE1, TMEM14A TOP2B, TPD52, TPM1, TRAF3IP3, TSPAN2, TTC9C, UBE2B, UBP1, UHRF2, VLDLR, VPS35, WASF1, WDFY1, WDR49, WDR68, WHDC1L1, WHDC1L2, ZBTB33, ZBTB44, ZF, ZNF207, ZNF519, ZNF658, and ZNF92 is substantially identical to the level of expression by the same type of cells in the presence of the glatiramer acetate drug substance under the same conditions; xxi) including the batch of the glatiramer acetate related drug substance in the production of the drug product if the level of expression of one or more genes selected from the group consisting of CCL2, CCL5, CXCL10, IL1RN, and MMP9 is substantially identical to the level of expression by the same type of cells in the presence of the glatiramer acetate drug substance under the same conditions; xxii) including the batch of the glatiramer acetate related drug substance in the production of the drug product if the level of expression of one or more genes selected from the group consisting of CCL5, CXCL10, and MMP9 is substantially identical to the level of expression by the same type of cells in the presence of the glatiramer acetate drug substance under the same conditions; xxiii) including the batch of the glatiramer acetate related drug substance in the production of the drug product if the level of expression of one or more genes selected from the group consisting of IL10 and CCL2 is substantially identical to the level of expression by the same type of cells in the presence of the glatiramer acetate drug substance under the same conditions; xxiv) including the batch of the glatiramer acetate related drug substance in the production of the drug product if the level of expression of one or more genes selected from the group consisting of IFNg, TNF, CCL3, CXCL8, and IL-10 is substantially identical to the level of expression by the same type of cells in the presence of the glatiramer acetate drug substance under the same conditions; xxv) including the batch of the glatiramer acetate related drug substance in the production of the drug product if the level of expression of one or more genes selected from the group consisting of MMP9, CCL2, CCL5, CXCL1, and IL1B is substantially identical to the level of expression by the same type of cells in the presence of the glatiramer acetate drug substance under the same conditions; xxvi) including the batch of the glatiramer acetate related drug substance in the production of the drug product if the level of expression of one or more genes selected from the group consisting of MMP9, level of expression by the same type of cells in the presence of the glatiramer acetate drug substance under the same conditions; xxix) including the batch of the glatiramer acetate related drug substance in the production of the drug product if the level of expression of one or more genes selected from the group consisting of CCL3, MMP9, CCL22, CCL24, CX3CL1, CCL20, CCL2, TNF, IL8, CCL13, CCL5, IL1B, CCL8, IL10, CXCL11, CXCL13, CXCL10, CCL7, CCL1, CXCL1, IFNg, CCL26, and MIF is substantially identical to the level of expression by the same type of cells in the presence of the glatiramer acetate drug substance under the same conditions; or xxx) including the batch of the glatiramer acetate related drug substance in the production of the drug product if the level of expression of one or more genes selected from the group consisting of CCL3, MMP9, CCL22, CCL24, CX3CL1, CCL20, CCL2, TNF, IL5, CCL13, CCL5, IL1B, CCL8, IL10, CXCL11, CXCL13, CXCL10, CCL7, CCL1, CXCL1, IFNg, CCL26, MIF, 1L16, IL6, CCL25, IL2, CCL19, CXCL2, CXCL9, and CXCL5 is substantially identical to the level of expression by the same type of cells in the presence of the glatiramer acetate drug substance under the same conditions;
b. i) including the batch of the glatiramer acetate related drug substance in the production of the drug product if the level of expression of one or more genes selected from the group consisting of ARPC4, CD84, CLU, HFE, and IL10 is upregulated relative to the level of expression by the same type of cells in the absence of the glatiramer acetate related drug substance under the same conditions; or ii) including the batch of the glatiramer acetate related drug substance in the production of the drug product if the level of expression of one or more genes selected from the group consisting of ABCF2, ACP6, AFG3L2, CHAF1A, COX11, LPHN1, NACA, OLAH, POLI, SEC31A, SNRPA1, SYNCRIP, TNFSF9, TOMM4O, TSHZ1, TSPAN13, UBAP2, VDAC2, and TSHZ1 is downregulated relative to the level of expression by the some type of cells in the absence of the glatiramer acetate related drug substance under the same conditions;
c. i) including the batch of the glatiramer acetate related drug substance in the production of the drug product if the level of expression of one or more genes selected from the group consisting of CCL2, CCL5, MMP1, MMP9, CXCL10, CARD15, CD14, ICAM1, BIRC3, THBD, NFKBIA, IL10, PRDM1 is substantially identical to the level of expression by the same type of cells in the presence of glatiramer acetate drug substance under the same conditions; ii) including the batch of the glatiramer acetate related drug substance in the production of the drug product if the level of expression of one or more genes selected from the group consisting of CISH and HSPD1 is substantially identical to the level of expression by the same type of cells in the presence of glatiramer acetate drug substance under the same conditions; iii) including the batch of the glatiramer acetate related drug substance in the production of the drug product if the level of expression of one or more genes selected from the group consisting of CC124, CCR1, CSF1R, CX3CR1, IL27, IFNGR1, IL2RG, and IL7R is not substantially identical to the level of expression by the same type of cells in the absence of the glatiramer acetate drug related substance under the same conditions; iv) including the batch of the glatiramer acetate related drug substance in the production of the drug product if the level of expression of PGRMC1 is upregulated or substantially identical to the level of expression by the same type of cells in the absence of the glatiramer acetate related drug substance under the same conditions; v) including the batch of the glatiramer acetate related drug substance in the production of the drug product if the level of expression of MMP14 is substantially identical to the level of expression by the same type of cells in the presence of glatiramer acetate drug substance under the same conditions; vi) including the batch of the glatiramer acetate related drug substance in the production of the drug product if the level of expression of IL1RN is upregulated to the level of expression by the same type of cells in the absence of the glatiramer acetate related drug substance under the same conditions; or vii) including the batch of the glatiramer acetate related drug substance in the production of the drug product if the level of expression of IL1B is substantially identical to the level of expression by the same type of cells in the presence of glatiramer acetate drug substance under the same conditions;
d. i) including the batch of the glatiramer acetate related drug substance in the production of the drug product if the level of expression of one or more genes selected from the group consisting of ADAMS, ADAMDEC1, AKR1C2, ANXA2, ANXA2P2, ARHGAP18, ARHGAP18, ARL6IP5, ARL6IP5, ATP2C1, BID, BIRC3, BTG1, CARD15, C1ORF21, C13ORF31, C5ORF13, C5ORF32, C9ORF130, CAST, CCL2, CCL5, CD14, CD300A, CD36, CD40, CD55, CD9, CENTA2, CHST11, COL6A1, CRYBB2, CXCL10, CYLD, DAB2, EBI3, EBI2, ECOP, EGF, FABP4, FXYD2, GHRL, GIMAP8, GLIPR1, G0S2, HMGB2, HNRPLL, ICAM1, ICAM2, IFIH1, IFNGR1, IL10, IL1ORA, IL411, INADL, ISG20, ITGB5, KIAA1505, KYNU, LACTB, LOC54103, LOC388344, LOC652751, LPAAT-THETA, LPXN, MAFB, MALT1, MFI2, MGC5618, MGLL, MITF, MLF1, MMP1, MMP9, MPEG1, MTSS1, MXD1, NT5E, NFKBIE, NFKBIA, NFE2L3, NFE2L3, OSBPL11, P2RX4, P2RY5, PLEKHO1, POPDC3, PLAUR, PRDM1, PSCDBP, PTX3, RAB27B, RCSD1, RPL13, SGIP1, SLC39A8, SNORD68, SRPX2, SRA1, SLIC1, SLAMF8, SLIC1, SOD2, STATH, STEAP1, SYNJ2, SYNJ2, TATDN3, TOMS, THBD, TNFAIP3, TNFAIP6, TNFRSF9, TNFSF13B, TPSAB1, TPSB2, TREM1, TXNL2, VPS33A, and VSNL1, is substantially identical to the level of expression by the same type of cells in the presence of the glatiramer acetate drug substance under the same conditions; or) including the batch of the glatiramer acetate related drug substance in the production of the drug product if the level of expression of one or more genes selected from the group consisting of ACTN4, BTBD14A, C14ORF10, CISH, CLK1, CRLF3, FAM62A, FBX045, GAPDHS, HDAC4, HIC2, HNRPD, HSPD1, LOC648342, MYB, NAPB, OXCT2, SERPINB2, SFRS14, SPFH1, STT3B, WDFY1, ZNF250, and ZNF566, is substantially identical to the level of expression by the same type of cells in the presence of the glatiramer acetate drug substance under the same conditions; or
e. i) including the batch of the glatiramer acetate related drug substance in the production of the drug product if the level of expression of one or more genes selected from the group consisting of AHRR, CCDC36, CYP1B1, DOC1, EPB41L3, GAS7, GPR68, NPTX1, PDCD6, and TIPARP is substantially identical to the level of expression by the same type of cells in the presence of the glatiramer acetate drug substance under the same conditions; or ii) including the batch of the glatiramer acetate related drug substance in the production of the drug product if the level of expression of one or more genes selected from the group consisting of ADRB2, COTL1, LOC285758, LOC644137, MALAT1, PRG1, RNF43, SAT1, THAP5, TIMP3, and TSC22D1 is substantially identical to the level of expression by the same type of cells in the presence of the glatiramer acetate drug substance under the same conditions.

161. The process of claim 101, wherein step (c) further comprises determining the level of expression of at least one gene of ACP6, AQP1, DAPP1, GBP2, GBP3, NME7, PLA2G4A, SKIL or TMEM158.

162. The process of claim 161, wherein if the level of expression of ABI2 is upregulated relative to the level of expression by the same type of cells in the absence of the glatiramer acetate related drug substance under the same conditions, and
if the level of expression of at least one gene of AQP1, DAPP1, GBP2, GBP3, NME7, PLA2G4A, SKIL or TMEM158 is upregulated relative to the level of expression by the same type of cells in the absence of the glatiramer acetate related drug substance under the same conditions, then including the batch of the glatiramer acetate related drug substance in the production of the drug product.

163. The process of claim 101, wherein step (c) further comprises determining the level of expression of at least one gene of ABCG1, ACP6, CAMTA1, CCDC85B, CXCR4, EPB41, FAM117A, FLOT2, GYPC, LPHN1, NR1D2, PATZ1 or TSHZ1.

164. The process of claim 163, wherein if the level of expression of ABI2 is upregulated relative to the level of expression by the same type of cells in the absence of the glatiramer acetate related drug substance under the same conditions, and
a. if the level of expression of at least one gene of ABCG1, ACP6, CAMTA1, CCDC85B, CXCR4, FAM117A, FLOT2, LPHN1 or NR1D2 is downregulated relative to the level of expression by the same type of cells in the absence of the glatiramer acetate related drug substance under the same conditions;
b. if the level of expression of at least one gene of GYPC or PATZ1 is not substantially identical to the level of expression by the same type of cells in the absence of the glatiramer acetate related drug substance under the same conditions; or
c. if the level of expression of EPB41 is substantially identical to the level of expression by the same type of cells in the presence of the glatiramer acetate drug substance under the same conditions,
then including the batch of the glatiramer acetate related drug substance in the production of the drug product.

165. The process of claim 101, wherein step (c) further comprises determining the level of expression of at least one gene of ACP6 or LPHN1.

166. The process of claim 165, wherein if the level of expression of ABI2 is upregulated relative to the level of expression by the same type of cells in the absence of the glatiramer acetate related drug substance under the same conditions, and
if the level of expression of at least one gene of ACP6 or LPHN1 is downregulated relative to the level of expression by the same type of cells in the absence of the glatiramer acetate related drug substance under the same conditions, then including the batch of the glatiramer acetate related drug substance in the production of the drug product.

167. The process of claim 159,
wherein step (c) further comprises i) determining the level of expression of at least one gene of ABCF2, AFG3L2, ALMS1, ARPC4, CALMS, CCDC64, CD84, CDC6, CHAF1A, CLU, COX11, DLGAP1, DTX4, FAM49B, FHL1, FNTB, GYPC, HFE, IL10, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, or ZFAND6; ii) determining the level of expression of at least one gene of C13ORF31, C14ORF10, C1ORF51, C1ORF63, CBR4, CB36, CD9, COL6A1, DAB2, GATA2, KIAA0907, LOC100506233, MCM6, MMP1, MS4A4A, MTSS1, PCMTD1, STK4, STX7, TAF15, TARP, TIA1, TMF1, TRGC2, TXNDC11, or ZCCHC7; or iii) determining the level of expression of at least one gene of ACTN4, BTBD14A, C14ORF10, CISH, CLK1, CRLF3, FAM62A, FBX045, GAPDHS, HDAC4, HIC2, HNRPD, HSPD1, LOC648342, MYB, NAPB, OXCT2, SERPINB2, SFRS14, SPFH1, STT3B, WDFY1, ZNF250, or ZNF566,
wherein all genes ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, IL10, LPHN1, NACA, OLAH, PATZ1, PDK1, POLI, REEP5, RPL5, RPS6KA2, SEC31A, SETBP1, SNRPA1, SYNCRIP, TNFSF9, TOMM40, TPM1, TSHZ1, TSPAN13, UBAP2, VAV3, VDAC2, and ZFAND6, are selected for determining the level of expression,
wherein all genes ABCF2, ABI2, ACP6, AFG3L2, ALMS1, ARPC4, CALM3, CCDC64, CD84, CDC6, CHAF1A, CLU, COX11, DLGAP1, DTX4, FAM49B, FHL1, FNTB, GYPC, HFE, LPHN1, OLAH, PATZ1, PDK1, POLI, REEFS, RPS6KA2, SEC31A, SETBP1, SNRPA1, SYNCRIP, TNFSF9, TOMM40, TPM1, TSPAN13, UBAP2, VAV3, VDAC2, and ZFAND6, are selected for determining the level of expression, and wherein in step (c)(i), if IL10 is the at least one gene selected in part(c)(1), then selecting at least a second different gene from other than IL10, or
wherein in step (c) (ii), if CARD15, CCL2, CCL5, CD14, IL10, THBD, or NFKBIA is the at least one of gene is selected in part (c)(ii), then selecting at least a second different gene from (c)(i) or c(ii) other than CARD15, CCL2, CCL5, CD14, IL10, THBD, or NFKBIA, and wherein if two or more genes are selected in step (c), then the second or additional gene selected is different from the other selected gene or genes.

168. The process of claim 159 comprising,
the step of (c)(vii) or (c)(viii),
wherein the level of expression is determined for all genes identified in Table 5 or Table 12 to be involved in one or more than one pathway, at least one pathway, two or more pathways, three or more pathways, four or more pathways, five or more pathways, six pathways;
wherein the level of expression is determined for at least one gene which is involved in only one pathway set forth in Table 5 or Table 12; or
wherein the level of expression is determined for at least two genes, at least three genes, at least tour genes, at least five genes, or at least six genes identified in Table 5 or Table 12 to be involved in the same pathway, or
the step of (c) (v) or (C) (vi),
wherein the level of expression is determined for all genes identified in Table 6 to be involved in one or more than one pathway, at least one pathway, two or more pathways, three or more pathways, four or more pathways, five or more pathways, six or more pathways, or at least one gene which is involved in only one pathway set forth in Table 6; or
wherein the level of expression is determined for at least two genes, at least three genes, at least four genes, at least five genes, or at least six genes identified in Table 6 to be involved in the same pathway.
